(19) United States
(12) Patent Application Publication
(10) Pub. No.: US 2015/0291598 A1
(43) Pub. Date: Oct. 15, 2015

(54) COMPOUNDS AND COMPOSITIONS FOR THE TREATMENT OF PARASITIC DISEASES

(71) Applicants: Arnab Kumar CHATTERJEE, San Diego, CA (US); Advait Suresh NAGLE, San Diego, CA (US); Prasuna PARASELLI, San Diego, CA (US); Seh Yong LEONG, Singapore (SG); Jason Thomas ROLAND, San Diego, CA (US); Pranab Kumar MISHRA, Holland, PA (US); Bryan KS YEUNG, Singapore (SG); Bin ZOU, Singapore (SG)

(72) Inventors: Arnab Kumar CHATTERJEE, San Diego, CA (US); Advait Suresh NAGLE, San Diego, CA (US); Prasuna PARASELLI, San Diego, CA (US); Seh Yong LEONG, Singapore (SG); Jason Thomas ROLAND, San Diego, CA (US); Pranab Kumar MISHRA, Holland, PA (US); Bryan KS YEUNG, Singapore (SG); Bin ZOU, Singapore (SG)

(73) Assignee: NOVARTIS AG, Basel (CH)

(21) Appl. No.: 14/443,604

(22) PCT Filed: Nov. 18, 2013
(86) PCT No.: PCT/US2013/070623

§ 371 Date: May 18, 2015

Related U.S. Application Data
(60) Provisional application No. 61/728,024, filed on Nov. 19, 2012.

(51) Int. Cl. C07D 487/04; A61K 31/5377; C07D 519/00; A61K 45/06; A61K 31/635; A61K 31/541; A61K 31/5415; A61K 31/519; A61K 31/538

(52) U.S. Cl. CPC C07D 487/04; A61K 31/519; A61K 31/5377; C07D 519/00; A61K 31/538; A61K 31/635; A61K 31/541; A61K 31/5415; A61K 45/06

(57) ABSTRACT

The present invention provides compounds of formula I: [INSERT FORMULA HERE] or a pharmaceutically acceptable salt, tautomer, or stereoisomer, thereof, wherein the variables are as defined herein. The present invention further provides pharmaceutical compositions comprising such compounds and methods of using such compounds for treating, preventing, inhibiting, ameliorating, or eradicating the pathology and/or symptomology of a disease, such as malaria, caused by a Plasmodium parasite.

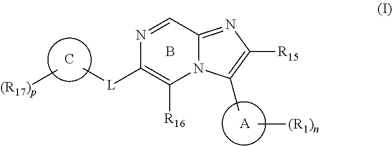

### CROSS-REFERENCED TO RELATED APPLICATIONS

This application claims the benefit of priority to U.S. Provisional Patent Application No. 61/728,024, filed 19 Nov. 2012, the full disclosure of which is expressively incorporated herein by reference in its entirety and for all purposes.

### BACKGROUND OF THE INVENTION

1. Field of the Invention

The invention provides a class of compounds, pharmaceutical compositions comprising such compounds and methods of using such compounds to treat or prevent malaria.

2. Background

Malaria is an infectious disease caused by four protozoan parasites: Plasmodium falciparum; Plasmodium vivax; Plasmodium ovale; and Plasmodium malaria. These four parasites are typically transmitted by the bite of an infected female Anopheles mosquito. Malaria is a problem in many parts of the world and over the last few decades the malaria burden has steadily increased. An estimated 1-3 million people die every year from malaria—mostly children under the age of 5. This increase in malaria mortality is due in part to the fact that Plasmodium falciparum, the deadliest malaria parasite, has acquired resistance against nearly all available antimalarial drugs, with the exception of the artemisinin derivatives. Further for true causal prophylaxis and interrupt transmission of the disease, prevention of liver stage development is crucial, because development of the proceeding infectious blood stage gametocytes would be block. A single drug effective against hepatichypnozoites, primaquine, is available, but its deployment is curtailed by its potential side effects.

Leishmaniasis is caused by one or more than 20 varieties of parasitic protozoa that belong to the genus Leishmania, and is transmitted by the bite of female sand flies. Leishmaniasis is endemic in about 88 countries, including many tropical and sub-tropical areas.

There are four main forms of Leishmaniasis. Visceral leishmaniasis, also called kala-azar, is the most serious form and is caused by the parasite Leishmania donovani. Patients who develop visceral leishmaniasis can die within months unless they receive treatment. The two main therapies for visceral leishmaniasis are the antimony derivatives sodium stibogluconate (Pentostam®) and meglumine antimoniate (Glucantim®). Sodium stibogluconate has been used for about 70 years and resistance to this drug is a growing problem. In addition, the treatment is relatively long and painful, and can cause undesirable side effects.

Human African Trypanosomiasis, also known as sleeping sickness, is a vector-borne parasitic disease. The parasites concerned are protozoa belonging to the Trypanosoma Genus. They are transmitted to humans by tsetse fly (Glossina Genus) bites which have acquired their infection from human beings or from animals harboring the human pathogenic parasites.

Chagas disease (also called American Trypanosomiasis) is another human parasitic disease that is endemic amongst poor populations on the American continent. The disease is caused by the protozoan parasite Trypanosoma cruzi, which is transmitted to humans by blood-sucking insects. The human disease occurs in two stages: the acute stage, which occurs shortly after infection and the chronic stage, which can develop over many years. Chronic infections result in various neurological disorders, including dementia, damage to the heart muscle and sometimes dilation of the digestive tract, as well as weight loss. Untreated, the chronic disease is often fatal.

The drugs currently available for treating Chagas disease are Nifurtimox and benznidazole. However, problems with these current therapies include their diverse side effects, the length of treatment, and the requirement for medical supervision during treatment. Furthermore, treatment is really only effective when given during the acute stage of the disease. Resistance to the two frontline drugs has already occurred. The antifungal agent Amphotericin b has been proposed as a second-line drug, but this drug is costly and relatively toxic.

In view of the foregoing, it is desirable to develop novel compounds as antiparasitic agents.

### SUMMARY OF THE INVENTION

The invention therefore provides a compound of formula (I):

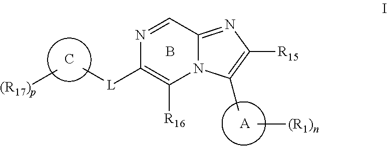

or a pharmaceutical acceptable salt, tautomer or stereoisomer thereof, wherein
n is 0, 1 or 2;
p is 0, 1, 2 or 3;
Ring A is selected from the group consisting of C₆₋₁₀aryl, C₅₋₁₀heteroaryl and fused bicyclyls comprising a C₅₋₆heterocycloalkyl fused to a phenyl;
Ring B represents the imidazo[1,2-a]pyrazine fused ring depicted in Formula 1;
Ring C is selected from the group consisting of phenyl, C₅₋₁₀heteroaryl, C₅₋₆cycloalkyl, C₅₋₆heterocycloalkyl, and fused bicyclyl comprising a C₅₋₆heterocycloalkyl fused to a phenyl;
L is selected from the group consisting of *—CH₂N(R₂)—, *—C(O)—, *—C(O)N(R₂)—, *—C(O)N(R₂)C(R₃ₐ)(R_{3b})—, *—N(R₂)C(R₃ₐ)(R_{3b})—, *—N(R₂)C(O)—, *—N(R₂)SO₂—, and C₁₋₆alkylene, wherein
   * represents the point of attachment of L to Ring B;
   R₂ is selected from the group consisting of hydrogen, C₁₋₄alkyl and R₀—C₁₋₄alkylene, wherein R₀ is selected from the group consisting of C₁₋₄alkyl, C₁₋₄alkoxy, amino, C₁₋₄alkylamino, C₅₋₆heteroaryl and C₅₋₆heterocycloalkyl, wherein the C₅₋₆heteroaryl and C₅₋₆heterocycloalkyl of R₀ are each unsubstituted or substituted with 1 to 2 substituents independently selected from the group consisting of C₁₋₄alkyl, halo and amino;
   R₃ₐ and R_{3b} are each independently selected from the group consisting of hydrogen and C₁₋₄alkyl, or R₃ₐ and R_{3b} is taken together with the carbon to which both attached to form a cyclopropyl;
each R₁ is independently selected from the group consisting of halo, cyano, —OR₄, —C(O)R₅, —C(O)NR₆R₇, —NR₈R₉, —NHC(O)R₁₀, —NHSO₂R₁₁, —SO₂R₁₂, C₁₋₆alkyl, phenyl, C₅₋₉heteroaryl, and C₄₋₆heterocycloalkyl, wherein
   R₄ is C₁₋₆alkyl or phenyl, wherein the C₁₋₆alkyl is unsubstituted or substituted by 1 to 3 substituents independently selected from the group consisting of halo, cyano, C₁₋₄alkyl, amino, di-C₁₋₄alkylamino, and —C(O)NH₂);
   R₅ is hydrogen, C₁₋₆alkyl or C₁₋₆alkoxy;
   R₆, R₈ and R₁₁ are each independently hydrogen or C₁₋₄alkyl;
   R₇ and R₉ are each independently selected from the group consisting of hydrogen, C₁₋₄alkyl, C₁₋₄alkoxy, and C₃₋₆cycloalkyl, wherein the C₁₋₄alkyl is unsubstituted or substituted with 1 to 3 substituents independently selected from the group consisting of amino, C₁₋₄alkyl, C₁₋₄alkylamino, di-C₁₋₄alkylamino, C₁₋₄alkoxycarbonylamino, and C₅₋₆heterocycloalkyl;
   R₁₀ is C₁₋₆alkyl, C₁₋₆alkoxy or C₃₋₆cycloalkyl, wherein the C₁₋₆alkyl of R₁₀ is unsubstituted or substituted by 1-2 substituents independently selected from amino and C₃₋₆cycloalkyl, and the C₃₋₆cycloalkyl of R₁₀ is unsubstituted or substituted by 1 to 2 substituents independently selected from the group consisting of amino and amino-C₁₋₄alkyl,
   R₁₂ is C₁₋₄alkyl, amino or C₁₋₄alkylamino,
   the C₁₋₆alkyl of R₁ is unsubstituted or substituted with 1-3 substituents independently selected from the group consisting of halo, cyano, methoxy, amino, C₁₋₄alkylamino, C₅₋₆cycloalkyl, and phenyl; and
   the phenyl, C₅₋₆heteroaryl and C₃₋₆heterocycloalkyl of R₁ are each independently unsubstituted or substituted with 1 to 2 substituents independently selected from the group consisting of C₁₋₄alkyl, amino, C₁₋₄alkylamino, —C(O)CH₃, and benzyl;
R₁₅ and R₁₆ are each independently hydrogen, C₁₋₄alkyl or haloC₁₋₄alkyl;
each R₁₇ is independently selected from the group consisting of cyano, halo, oxo, OR₁₈, —C(O)R₁₉, —NR₂₀R₂₁, —SO₂R₂₂, —SO₂NHR₂₃, C₁₋₄alkyl, phenyl, C₅₋₉heteroaryl, C₃₋₆cycloalkyl and C₄₋₆heterocycloalkyl, wherein
   R₁₈ is selected from the group consisting of hydrogen, C₁₋₄alkyl, haloC₁₋₄alkyl and phenyl;
   R₁₉ is selected from the group consisting of hydrogen, C₁₋₄alkyl, amino, and C₁₋₄alkylamino;
   R₂₀, R₂₁ and R₂₂ are each independently hydrogen or C₁₋₄alkyl;
   R₂₃ is hydrogen, C₁₋₄alkyl, or C₅₋₆heteroaryl
   the C₁₋₄alkyl of R₁₇ is unsubstituted or substituted with 1-3 substituents independently selected from the group consisting of halo, C₁₋₄alkoxy and amino; and
   the phenyl, C₅₋₉heteroaryl, C₃₋₆cycloalkyl and C₄₋₆heterocycloalkyl of R₁₇ are each independently unsubstituted or substituted by 1 to 2 substituents independently selected from the group consisting of C₁₋₄alkyl, halo-C₁₋₄alkyl, C₁₋₄alkoxy-C₁₋₄alkyl, and C₁₋₄alkoxy.
   the C₁₋₄alkyl of R₁₇ is unsubstituted or substituted with 1-3 substituents independently selected from the group consisting of halo, C₁₋₄alkoxy and amino; and
   the phenyl, C₅₋₉heteroaryl, C₃₋₆cycloalkyl and C₄₋₆heterocycloalkyl of R₁₇ are each independently unsubstituted or substituted with 1 to 2 substituents independently selected from the group consisting of C₁₋₄alkyl, halo-C₁₋₄alkyl, C₁₋₄alkoxy-C₁₋₄alkyl, and C₁₋₄alkoxy.

In a second aspect, the present invention provides a pharmaceutical composition which contains a compound selected from Formula I, 1A or a N-oxide derivative, individual isomers and mixture of isomers thereof; or a pharmaceutically acceptable salt thereof, in admixture with one or more suitable excipients.

In a third aspect, the present invention provides a method of treating a disease in an animal in which a compound of the invention can prevent, inhibit or ameliorate the pathology and/or symptomology of disease caused by a parasite (such as, for example, Plasmodium falciparum, Plasmodium vivax, Plasmodium ovale, Plasmodium malaria, Trypanosoma cruzi or a parasite of the Leishmania genus such as, for example, Leishmania donovani) which method comprises administering to the animal a therapeutically effective amount of a compound selected from Formula I, 1A or a N-oxide derivative, individual isomers and mixture of isomers thereof, or a pharmaceutically acceptable salt thereof.

In a fourth aspect, the present invention provides a compound according to formula 1, 1A, or a pharmaceutically acceptable salt thereof, for treating, preventing, inhibiting, ameliorating, or eradicating the pathology and/or symptomology of a disease caused by a parasite (such as, for example, Plasmodium falciparum, Plasmodium vivax, Plasmodium ovale, Plasmodium malaria, Trypanosoma cruzi or a parasite of the Leishmania genus such as, for example, Leishmania donovani). Particularly, the parasite is a Plasmodium which can be at the blood stages or at the hepatic stages, and the disease is malaria.

In a fifth aspect, the present invention provides the use of a compound selected from Formula I or Formula 1a in the manufacture of a medicament for treating a disease caused by a parasite in an animal. The disease may be malaria, leishmaniasis and/or Chagas disease.

In a sixth aspect, the present invention provides a process for preparing compounds selected from Formula I, Formula 1a and the N-oxide derivatives, prodrug derivatives, individual isomers and mixture of isomers thereof, and the pharmaceutically acceptable salts thereof.

Unless specified otherwise, the term “compounds of the present invention” refers to compounds of Formula (I) and subformulae thereof, salts of the compound, hydrates or solvates of the compounds, salts, as well as all stereoisomers (including diastereoisomers and enantiomers), tautomers and isotopically labeled compounds (including deuterium substitutions). Compounds of the present invention further comprise polymorphs of compounds of formula I (or subformulae thereof) and salts thereof.

### DETAILED DESCRIPTION OF THE INVENTION

### Definitions

For purposes of interpreting this specification, the following definitions will apply and whenever appropriate, terms used in the singular will also include the plural and vice versa.

“Acyl” as used herein refers to the radical —C(═O)Rₐ, where Rₐ is hydrogen or a non-hydrogen substituent on the carbonyl carbon, forming different carbonyl-containing groups including, but are not limited to, acids, acid halides, aldehydes, amides, esters, and ketones.

“Alkoxy” as used herein refers the radical —O-alkyl, wherein the alkyl is as defined herein. C_{X}alkoxy and C_{X-Y}alkoxy as used herein describe alkoxy groups where X and Y indicate the number of carbon atoms in the alkyl chain. Representative examples of C₁₋₁₀alkoxy include, but are not limited to, methoxy, ethoxy, propoxy, 2-propoxy, butoxy, tert-butoxy, pentyloxy, hexyloxy, heptyloxy, octyloxy and decyloxy. The alkyl portion of the alkoxy may be optionally substituted, and the substituents include those described for the alkyl group below.

“Alkyl” as used herein refers to a fully saturated branched or unbranched hydrocarbon chain having up to 10 carbon atoms. C_{X} alkyl and C_{X-Y} alkyl as used herein describe alkyl groups where X and Y indicate the number of carbon atoms in the alkyl chain. For example, C₁₋₁₀ alkyl refers to an alkyl radical as defined above containing one to ten carbon atoms. C₁₋₁₀ alkyl includes, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like. Alkyl represented along with another radical like arylalkyl, heteroarylalkyl, alkoxyalkyl, alkoxyalkyl, alkylamino, where the alkyl portion shall have the same meaning as described for alkyl and is bonded to the other radical. For example, (C₆₋₁₀)aryl(C₁₋₃)alkyl includes, benzyl, phenylethyl, 1-phenylethyl, 3-phenylpropyl, 2-thienylmethyl, 2-pyridinylmethyl and the like.

Unless stated otherwise specifically in the specification, an alkyl group may be unsubstituted or substituted by one or more substituents to the extent that such substitution makes sense chemically. Typical substituents include, but are not limited to halo, hydroxyl, alkoxy, cyano, amino, acyl, aryl, arylalkyl, and cycloalkyl, or an heteroforms of one of these groups, and each of which can be substituted by the substituents that are appropriate for the particular group.

“Alkenyl” as used herein refers to a straight or branched, hydrocarbon chain having up to 10 carbon atoms and at least one carbon-carbon double bond. C_{X}alkenyl and C_{X-Y}alkenyl as used herein describe alkenyl groups where X and Y indicate the number of carbon atoms in the alkenyl chain. Examples of C₂₋₇alkenyl include vinyl, allyl, isopropenyl, pentenyl, hexenyl, heptenyl, 1-propenyl, 2-butenyl, 2-methyl-2-butenyl, and the like. The alkenyl may be optionally substituted, and the substituents include those described for the alkyl group descried herein.

“Alkynyl” as used herein refers to a straight or branched, hydrocarbon chain having up to 10 carbon atoms and at least one carbon-carbon triple bond. C_{X}alkenyl and C_{X-Y}alkenyl as used herein describe alkynyl groups, where X and Y indicate the number of carbon atoms in the alkynyl chain. For example, C₂₋₇alkenyl include, but are not limited to, ethynyl, propargyl, 3-methyl-1-pentynyl, 2-heptynyl and the like. An alkynyl may be optionally substituted, and the substituents include those described for the alkyl group described herein.

“Alkylene” as used herein refers to a divalent alkyl group defined herein. Examples of C₁₋₁₀alkylene includes, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene and n-decylene. An alkylene group may be optionally substituted, and the substituents include those described for the alkyl group described herein.

“Alkenylene” as used herein refers to a divalent alkenyl group defined herein. Examples of C₁₋₃alkenylene include, but are not limited to, ethene-1,2-diyl, propene-1,3-diyl, and methylene-1,1-diyl. An alkenylene may be optionally substituted, and the substituents include those described for the alkyl group described herein.

“Alkynylene” as used herein refers to a divalent alkynyl group defined herein. Examples of alkynylene include ethyne-1,2-diylene, propyne-1,3-diylene, and the like. An alkynylene may be optionally substituted, and the substituents include those described for the alkyl group described herein.

“Amino” as used herein refers to the radical —NH₂. When an amino is described as “substituted” or “optionally substituted”, the term includes NR′R″ wherein each R′ and R″ is independently H, or is an alkyl, alkenyl, alkynyl, acyl, aryl, aryl, cycloalkyl, arylalkyl cycloalkylalkyl group or a heteroform of one of these groups, and each of the alkyl, alkenyl, alkynyl, acyl, aryl, arylalkyl or groups or heteroforms of one of these groups, each of which is optionally substituted with the substituents described herein as suitable for the corresponding group.

The term “amino” also includes forms wherein R′ and R″ are linked together to form a 3-8 membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Alkylamino” as used herein refers to the radical —NRₐR_{b}, where at least one of, or both, Rₐ and R_{b} are an alkyl group as described herein. An C₁₋₄alkylamino group includes —NHC₁₋₄alkyl and —N(C₁₋₄alkyl)₂; e.g., —NHCH₃, —N(CH₃)₂, —NH(CH₂CH₃), —N(CH₂CH₃)₂, and the like.

“Aromatic” as used herein refers to a moiety wherein the constituent atoms make up an unsaturated ring system, where all atoms in the ring system are sp² hybridized and the total number of pi electrons is equal to 4n+2. An aromatic ring may be such that the ring atoms are only carbon atoms or may include carbon and non-carbon atoms (see Heteroaryl).

“Aryl” as used herein refers to a 6-14 membered monocyclic or polycyclic aromatic ring assembly where all the ring atoms are carbon atoms. Typically, the aryl is a 6 membered monocyclic, a 10-12 membered bicyclic or a 14-membered fused tricyclic aromatic ring system. C_{X}aryl and C_{X-Y}aryl as used herein describe an aryl group where X and Y indicate the number of carbon atoms in the ring system. C₆₋₁₄aryls include, but are not limited to, phenyl, biphenyl, naphthyl, azulenyl, and anthracenyl.

An aryl may be unsubstituted or substituted by 1-5 (such as one, or two, or three) substituents independently selected from the group consisting of hydroxy, thiol, cyano, nitro, C₁₋₄alkyl, C₁₋₄alkenyl, C₁₋₄alkynyl, C₁₋₄alkoxy, thioC₁₋₄alkyl, C₁₋₄alkenyloxy, C₁₋₄alkynyloxy, halogen, C₁₋₄alkylcarbonyl, carboxy, C₁₋₄alkoxycarbonyl, amino, C₁₋₄alkylamino, di-C₁₋₄alkylamino, C₁₋₄alkylaminocarbonyl, di-C₁₋₄alkylaminocarbonyl, C₁₋₄alkylcarbonylamino, C₁₋₄alkylcarbonyl(C₁₋₄ alkyl)amino, sulfonyl, sulfamoyl, alkylsulfamoyl, C₁₋₄alkylaminosulfonyl, aryl, heteroaryl, cycloalkyl and heterocycloalkyl, wherein each of the afore-mentioned substitutents may be further substituted by one or more substituents independently selected from halogen, alkyl, hydroxyl or C₁₋₄alkoxy groups.

When an “aryl” is represented along with another radical like “arylalkyl”, “aryloxyalkyl”, “aryloxycarbonyl”, “aryloxy-carbonylalkyl”, the aryl portion shall have the same meaning as described in the above-mentioned definition of “aryl”.

“Aryloxy” as used herein, refers to the radical —O-aryl, wherein aryl is as defined herein.

“Bicyclic” or “bicyclyl” as used here in refers to a ring assembly of two rings where the two rings are fused together, linked by a single bond or linked by two bridging atoms. The rings may be a carbocyclyl, a heterocyclyl, or a mixture thereof.

“Bridging ring” as used herein refers to a polycyclic ring system where two ring atoms that are common to two rings are not directly bound to each other. One or more rings of the ring system may also comprise heteroatoms as ring atoms. Non-exclusive examples of bridging rings include norbornanyl, 7-oxabicyclo[2.2.1]heptanyl, adamantanyl, and the like.

“Carbamoyl” as used herein refers to the radical —C(O)NRₐ— where Rₐ is H, or is an alkyl, alkenyl, alkynyl, acyl, aryl, or arylalkyl group or a heteroform of one of these groups, and each of the alkyl, alkenyl, alkynyl, acyl, aryl, arylalkyl or heteroforms of one of these groups is optionally substituted with the substituents described herein as suitable for the corresponding group.

“Carbamate” as used herein refers to the radical —OC(O)NRₐR_{b} where Rₐ and R_{b} are each independently H, or is an alkyl, alkenyl, alkynyl, acyl, aryl, or arylalkyl group or a heteroform of one of these groups, and each of the alkyl, alkenyl, alkynyl, acyl, aryl, arylalkyl or heteroforms of one of these groups is optionally substituted with the substituents described herein as suitable for the corresponding group.

“Cycloalkyl”, as used herein, means a radical comprising a non-aromatic, saturated or partially unsaturated, monocyclic, bicyclic, tricyclic, fused, bridged or spiro polycyclic hydrocarbon ring system of 3-20 carbon atoms. C_{X}cycloalkyl and C_{X-Y}cycloalkyl are typically used where X and Y indicate the number of carbon atoms in the ring assembly. For example, C₃₋₆cycloalkyl includes cyclopropyl, cyclobutyl, cyclopentyl, cyclohexyl, cyclohexenyl, 2,5-cyclohexadienyl.

Exemplary monocyclic cycloalkyl include, but are not limited to, cyclopropyl, cyclobutyl, cyclopentyl, cyclopentenyl, cyclohexyl and cyclohexenyl and the like.

Exemplary bicyclic cycloalkyls include bornyl, norbornanyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl. Exemplary tricyclic cycloalkyl groups include, for example, adamantyl.

A cycloalkyl may be unsubstituted or substituted by one, or two, or three, or more substituents independently selected from the group consisting of hydroxyl, thiol, cyano, nitro, oxo, alkylimino, C₁₋₄alkyl, C₁₋₄alkenyl, C₁₋₄alkynyl, C₁₋₄alkoxy, C₁₋₄thioalkyl, C₁₋₄alkenyloxy, C₁₋₄alkynyloxy, halogen, C₁₋₄alkylcarbonyl, carboxy, C₁₋₄alkoxycarbonyl, amino, C₁₋₄alkylamino, di-C₁₋₄ alkylamino, C₁₋₄alkylaminocarbonyl, di-C₁₋₄alkylaminocarbonyl, C₁₋₄alkylcarbonylamino, C₁₋₄ alkylcarbonyl(C₁₋₄alkyl)amino, sulfonyl, sulfamoyl, alkylsulfamoyl, C₁₋₄alkylaminosulfonyl where each of the afore-mentioned hydrocarbon groups (e.g., alkyl, alkenyl, alkynyl, alkoxy residues) may be further substituted by one or more residues independently selected at each occurrence from halogen, hydroxyl or C₁₋₄alkoxy groups.

“Cycloalkylene”, as used herein, refers to a divalent radical comprising a cycloalkyl ring assembly as defined herein.

“Cycloalkoxy”, as used herein, refers to —O-cycloalkyl, wherein the cycloalkyl is defined herein. Representative examples of C₃₋₁₂cycloalklyoxy include, but are not limited to, monocyclic groups such as cyclopropoxy, cyclobutoxy, cyclopentyloxy, cyclopentenyloxy, cyclohexyloxy and cyclohexenyloxy and the like. Exemplary bicyclic hydrocarbon groups include bornyloxy, indyloxy, hexahydroindyloxy, tetrahydronaphthyloxy, decahydronaphthyloxy, bicyclo[2.1.1]hexyloxy, bicyclo[2.2.1]heptyloxy, bicyclo[2.2.1]heptenyloxy, 6,6-dimethylbicyclo[3.1.1]heptyloxy, 2,6,6-trimethylbicyclo[3.1.1]heptyloxy, bicyclo[2.2.2]octyloxy and the like. Exemplary tricyclic hydrocarbon groups include, for example, adamantyloxy.

“Cyano”, as used herein, refers to the radical —CN.

“EC₅₀”, refers to the molar concentration of an inhibitor or modulator that produces 50% efficacy.

“Fused ring”, as used herein, refers to a multi-ring assembly wherein the rings comprising the ring assembly are so linked that the ring atoms that are common to two rings are directly bound to each other. The fused ring assemblies may be saturated, partially saturated, aromatics, carbocyclics, heterocyclics, and the like. Non-exclusive examples of common fused rings include decalin, naphthalene, anthracene, phenanthrene, indole, benzofuran, purine, quinoline, and the like.

“Halo” or “halogen” as used herein refers to fluoro, chloro, bromo, and iodo.

“Haloalkyl”, or halo-substituted-alkyl” as used herein, refers to an alkyl as defined herein, which is substituted by one or more halo atoms defined herein. The haloalkyl can be mono-haloalkyl, dihaloalkyl or polyhaloalkyl including perhaloalkyl. A monohaloalkyl can have one iodo, bromo, chloro or fluoro within the alkyl group. Dihaloalky and polyhaloalkyl groups can have two or more of the same halo atoms or a combination of different halo groups within the alkyl. C_{X}haloalkyl and C_{X-Y}haloalkyl are typically used where X and Y indicate the number of carbon atoms in the alkyl chain. Non-limiting examples of C₁₋₄haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A C₁₋₄perhaloalkyl group refers to a C₁₋₄alkyl group having all hydrogen atoms replaced with halo atoms.

“Heteroaryl”, as used herein, refers to a 5-14 membered ring assembly (e.g., a 5-7 membered monocycle, an 8-10 membered bicycle, or a 13-14 membered tricyclic ring system) having 1 to 8 heteroatoms selected from N, O and S as ring atoms and the remaining ring atoms are carbon atoms. The nitrogen atoms of such heteroaryl rings can be optionally quaternerized and the sulfur atoms of such heteroaryl rings can be optionally oxidized. C_{X}heteroaryl and C_{X-Y}heteroaryl as used herein describe heteroaryls where X and Y indicate the number of ring atoms in the heteroaryl ring. Typical C₅₋₇heteroaryl groups include thienyl, furanyl, imidazolyl, pyrazolyl, pyrrolyl, pyrrolinyl, thiazolyl, 1,3,4-thiadiazolyl, isothiazolyl, oxazolyl, oxadiazole isoxazolyl, triazolyl, tetrazolyl, pyridyl, pyridazinyl, pyrazinyl, pyrazinyl, pyrimidinyl, and the like.

Bicyclic or tricyclic C₈₋₁₄heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2,3-b]pyridine, quinazolinyle, pteridinyl, indolizine, imidazo[1,2a]pyridine, quinoline, quinolinyl, isoquinoline, phthalazine, quinoxaline, naphthyridine, naphthyridinyl, quinolizine, indolyl, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3-c]pyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, purinyl, carbazole, acridine, phenazine, phenothiazene, phenoxazine, 1,2-dihydropyrrolo[3,2,1-hi]indole, indolizine, pyrido[1,2-a]indole and 2(1H)-pyridinone.

A heteroaryl may be unsubstituted or substituted with one or more substituents independently selected from hydroxyl, thiol, cyano, nitro, C₁₋₄alkyl, C₁₋₄alkenyl, C₁₋₄alkynyl, C₁₋₄alkoxy, thioC₁₋₄alkyl, C₁₋₄alkenyloxy, C₁₋₄alkynyloxy, halogen, C₁₋₄alkylcarbonyl, carboxy, C₁₋₄alkoxycarbonyl, amino, C₁₋₄alkylamino, di-C₁₋₄alkylamino, C₁₋₄alkylaminocarbonyl, di-C₁₋₄alkylaminocarbonyl, C₁₋₄alkylcarbonylamino, C₁₋₄alkylcarbonyl(C₁₋₄alkyl)amino, sulfonyl, sulfamoyl, alkylsulfamoyl, C₁₋₄alkylaminosulfonyl where each of the afore-mentioned hydrocarbon groups (e.g., alkyl, alkenyl, alkynyl, alkoxy residues) may be further substituted by one or more residues independently selected at each occurrence from halogen, hydroxyl or C₁₋₄alkoxy groups.

When a heteroaryl is represented along with another radical like “heteroaryloxy”, “heteroaryloxyalkyl”, “heteroaryloxycarbonyl”, the heteroaryl portion shall have the same meaning as described in the above-mentioned definition of “heteroaryl”.

“Heteroaryloxy”, as used herein, refers to an —O-heteroaryl group, wherein the heteroaryl is as defined in this application.

“Heteroatom”, as used herein, refers to an atom that is not a carbon atom. Particular examples of heteroatoms include, but are not limited to nitrogen, oxygen, and sulfur.

“Heterocycloalkyl”, as used herein, refers to a 4-20 membered, non-aromatic, saturated or partially unsaturated, monocyclic or polycyclic ring system, comprising 1-8 heteroatoms as ring atoms and that the remaining ring atoms are carbon atoms. The heteroatoms are selected from N, O, and S, preferably 0 and N. The nitrogen atoms of the heterocycloalkyl can be optionally quaternerized and the sulfur atoms of the heterocycloalkyl can be optionally oxidized. The heterocycloalkyl can include fused or bridged rings as well as spirocyclic rings. C_{X}heterocycloalkyl and C_{X-Y}heterocycloalkyl are typically used where X and Y indicate the number of ring atoms in the ring. Typically, the heterocycloalkyl is 4-8-membered monocyclic ring containing 1 to 3 heteroatoms, a 7 to 12-membered bicyclic ring system containing 1-5 heteroatoms, or a 10-15-membered tricyclic ring system containing 1 to 7 heteroatoms. Examples of C₄₋₆heterocycloalkyl include azetidinyl, tetrahydrofuran (THF), dihydrofuran, 1,4-dioxane, morpholine, 1,4-dithiane, piperazine, piperidine, 1,3-dioxolane, imidazolidine, imidazoline, pyrazolidinyl, pyrroline, pyrrolidine, tetrahydropyran, dihydropyran, oxathiolane, dithiolane, 1,3-dioxane, 1,3-dithiane, oxathiane, thiomorpholine, and the like

A heterocycloalkyl may be unsubstituted or substituted with 1-5 substituents (such as one, or two, or three) each independently selected from hydroxyl, thiol, cyano, nitro, oxo, alkylimino, C₁₋₄alkyl, C₁₋₄alkenyl, C₁₋₄alkynyl, C₁₋₄alkoxy, C₁₋₄thioalkyl, C₁₋₄alkenyloxy, C₁₋₄alkynyloxy, halogen, C₁₋₄alkylcarbonyl, carboxy, C₁₋₄alkoxycarbonyl, amino, C₁₋₄alkylamino, di-C₁₋₄alkylamino, C₁₋₄alkylaminocarbonyl, di-C₁₋₄alkylaminocarbonyl, C₁₋₄alkylcarbonylamino, C₁₋₄alkylcarbonyl(C₁₋₄ alkyl)amino, sulfonyl, sulfamoyl, alkylsulfamoyl, C₁₋₄alkylaminosulfonyl where each of the afore-mentioned hydrocarbon groups (e.g., alkyl, alkenyl, alkynyl, alkoxy residues) may be further substituted by one or more residues independently selected at each occurrence from halogen, hydroxyl or C₁₋₄alkoxy groups.

When a heterocycloalkyl forms part of other groups like “heterocycloalkyl-alkyl”, “heterocycloalkoxy”, “heterocycloalkyl-aryl”, the heteroaryl portion shall have the same meaning as described in the above-mentioned definition of “heteroaryl”

“Heterocycloalkylene”, as used herein, refers to a cycloalkylene, as defined in this application, provided that one or more of the ring member carbon atoms is replaced by a heteroatom.

“Heterocycloalkyl fused to a phenyl” as used herein, refers to a bicyclic fused ring system that one of the ring is heterocycloalkyl as defined above and the other ring is a phenyl. A heterocycloalkyl fused to a phenyl includes but are not limited to benzo[b][1,4]oxazinyl, oxo-benzo[b][1,4]oxazinyl, tetrahydroquinoxalinyl, tetrahydroquinolinyl, indolinyl, benzo[d]imidazolyl, and the like.

“Heterocyclyl”, “heterocycle” or “heterocyclo”, as used herein, refers to a 3-20 membered, monocyclic or polycyclic ring system containing at least one heteroatom moiety selected from the group consisting of N, O, SO, SO₂, (C═O), and S, and preferably N, O, S, optionally containing one to four additional heteroatoms in each ring. C_{X}heterocyclyl and C_{X-Y}heterocyclyl are typically used where X and Y indicate the number of ring atoms in the ring system. Unless otherwise specified, a heterocyclyl may be saturated, partially unsaturated, aromatic or partially aromatic.

Hydroxy, as used herein, refers to the radical —OH.

“Hydroxyalkyl” or “hydroxyl-substituted alkyl” as used herein, refers to an alkyl as defined herein, having one or more of the available hydrogen of the alkyl replaced by a hydroxyl group. For example, a hydroxyC₁₋₄alkyl includes, but are not limited to, —CH₂CH₂OH, —CH(OH)CH₂CH₂OH, —CH(OH)CH₂CH(OH)CH₃.

“Nitro”, as used herein, refers to the radical —NO₂.

“Oxo”, as used herein, refers to the divalent radical ═O

“Protected derivatives” means derivatives of inhibitors in which a reactive site or sites are blocked with protecting groups. Protected derivatives are useful in the preparation of inhibitors or in themselves may be active as inhibitors. Examples of protected group includes, but are not limited to, acetyl, tetrahydropyran, methoxymethyl ether, β-methoxyethoxymethyl ether, ρ-methoxybenzyl, methylthiomethyl ether, pivaloyl, silyl ether, carbobenzyloxy, benzyl, tert-butoxycarbonyl, ρ-methoxyphenyl, 9-fluorenylmethyloxycarbonyl, acetals, ketals, acylals, dithianes, methylesters, benzyl esters, tert-butyl esters, and silyl esters. A comprehensive list of suitable protecting groups can be found in T. W. Greene, Protecting Groups in Organic Synthesis, 3rd edition, John Wiley & Sons, Inc. 1999.

“Unsubstituted or substituted” or “optionally substituted” as used herein indicate the substituent bound on the available valance of a named group or radical. “Unsubstituted” as used herein indicates that the named group or radical will have no further non-hydrogen substituents. “Substituted” or “optionally substituted” as used herein indicates that at least one of the available hydrogen atoms of named group or radical has been (or may be) replaced by a non-hydrogen substituent.

“Substituted terminally” as used herein referred to a substituent replacing a hydrogen at a terminal position of the parent molecule. For example C₁₋₄alkyl substituted terminally by an amino means —C₁₋₄alkylene-amino, which includes —(CH₂)—NH₂, —(CH₂)₂—NH₂, —(CH₂)₃—NH₂, —(CH₂)CH₂(CH₂—NH₂), —(CH₂)₄—NH₂, —C(CH₂)(CH₂CH₂—NH₂)—C(CH₃)₂(CH₂—NH₂), and the like.

Unless otherwise specified, examples of substituents may include, but are not limited to, halo, nitro, cyano, thio, oxy, hydroxy, carbonyloxy, C₁₋₆alkoxy, C₆₋₁₀aryloxy, heteroC₅₋₁₀aryloxy, carbonyl, oxycarbonyl, aminocarbonyl, amino, C₁₋₆alkylamino, sulfonamido, imino, sulfonyl, sulfinyl, C₁₋₆alkyl, C₁₋₆haloalkyl, hydroxyC₁₋₆alkyl, carbonylC₁₋₆alkyl, thiocarbonylC₁₋₁₀alkyl, sulfonylC₁₋₆alkyl, sulfinylC₁₋₆alkyl, C₁₋₁₀azaalkyl, iminoC₁₋₆alkyl, C₃₋₁₂cycloalkylC₁₋₆alkyl, C₄₋₁₅heterocycloalkylC₁₋₆alkyl, C₅₋₁₀arylC₁₋₆alkyl, C₅₋₁₀heteroarylC₁₋₆alkyl, C₁₀₋₁₂bicycloarylC₁₋₆alkyl, C₉₋₁₂heterobicycloarylC₁₋₆alkyl, C₃₋₁₂cycloalkyl, C₄₋₁₂heterocycloalkyl, C₉₋₁₂bicycloalkyl, C₃₋₁₂heterobicycloalkyl, C₄₋₁₂aryl, heteroC₁₋₁₀aryl, C₉₋₁₂bicycloaryl and C₄₋₁₂heterobicycloaryl.

“Sulfamoyl” as used herein refers to the radical —S(O)₂NRₐR_{b} where Rₐ and R_{b} are independently H, or is an alkyl, alkenyl, alkynyl, acyl, aryl, aryl, cycloalkyl, arylalkyl cycloalkylalkyl group or a heteroform of one of these groups, and each of the alkyl, alkenyl, alkynyl, acyl, aryl, arylalkyl groups or heteroforms of one of these groups, is optionally substituted with the substituents described herein as suitable for the corresponding group.

“Sulfanyl” as used herein, means the radical —S—.

“Sulfinyl”, as used herein, means the radical —S(O)—. It is noted that the term “sulfinyl” when referring to a monovalent substituent can alternatively refer to a substituted sulfinyl group, —S(═O)R, where R is hydrogen or a non-hydrogen substituent on the sulfur atom forming different sulfinyl groups including sulfinic acids, sulfinamides, sulfinyl esters, and sulfoxides.

“Sulfonyl”, as used herein, means the radical —S(O)₂—. It is noted that the term “sulfonyl” when referring to a monovalent substituent can alternatively refer to a substituted sulfonyl group, —S(═O)₂R, where R is hydrogen or a non-hydrogen substituent on the sulfur atom forming different sulfonyl groups including sulfonic acids, sulfonamides, sulfonate esters, and sulfones.

“Thiocarbonyl”, as used herein, refers to the radical —C(═S)—. It is noted that the term thiocarbonyl when referring to a monovalent substituent can alternatively refer to a substituted thiocarbonyl group, —C(═S)R, where R is hydrogen or a non-hydrogen substituent on the carbon atom forming different thiocarbonyl groups including thioacids, thioamides, thioesters, and thioketones.

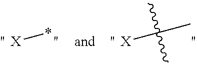

are symbols denoting the point of attachment of X, to other part of the molecule.

Any definition herein may be used in combination with any other definition to describe a composite structural group. By convention, the trailing element of any such definition is that which attaches to the parent moiety. For example, the composite group alkoxyalkyl would represent an alkoxy group attached to the parent molecule through an alkyl group.

It is noted in regard to all of the definitions provided herein that the definitions should be interpreted as being open ended in the sense that further substituents beyond those specified may be included. Hence, a C₂alkyl indicates that there is one carbon atom but does not indicate what are the substituents on the carbon atom. Hence, a C₁alkyl comprises methyl (i.e., —CH₃) as well as —CRₐR_{b}R_{c} where Rₐ, R_{b}, and R_{c} may each independently be hydrogen or any other substituent where the atom attached to the carbon is not a hydrogen atom. Hence, —CF₃, —CH₂OH and —CH₂CN, for example, are all C₁alkyls.

### DESCRIPTION OF THE PREFERRED EMBODIMENTS

The invention provides a novel class of compounds, pharmaceutical compositions comprising such compounds and methods of using such compounds to treat or prevent diseases or disorders associated with a parasite. In particular, the compounds can be used to treat malaria, leishmaniasis and/or Chagas disease. The compounds of the invention are effective in inhibiting, ameliorating, or eradicating the pathology and/or symptomology of the parasite at both the blood stage and hepatic stage.

In a first embodiment, the compounds of the invention, or a pharmaceutical acceptable salt, tautomer or stereoisomer thereof, are of Formula I:

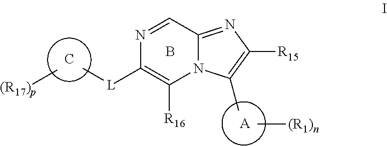

or a pharmaceutical acceptable salt, tautomer or stereoisomer thereof, wherein
   n is 0, 1 or 2;
   p is 0, 1, 2 or 3;
   Ring A is selected from the group consisting of C₆₋₁₀aryl, C₅₋₁₀heteroaryl and fused bicyclyls comprising a C₅₋₆heterocycloalkyl fused to a phenyl;
   Ring B represents the imidazo[1,2-a]pyrazine fused ring depicted in Formula 1;
   Ring C is selected from the group consisting of phenyl, C₅₋₁₀heteroaryl, C₅₋₆cycloalkyl, C₅₋₆heterocycloalkyl, and fused bicyclyl comprising a C₅₋₆heterocycloalkyl fused to a phenyl;
   L is selected from the group consisting of *—CH₂N(R₂)—, *—C(O)—, *—C(O)N(R₂)—, *—C(O)N(R₂)C(R₃ₐ)(R_{3b})—, *—N(R₂)C(R₃ₐ)(R_{3b})—, *—N(R₂)C(O)—, *—N(R₂)SO₂—, and C₁₋₆alkylene, wherein
      * represents the point of attachment of L to Ring B;
      R₂ is selected from the group consisting of hydrogen, C₁₋₄alkyl and R₀—C₁₋₄alkylene, wherein R₀ is selected from the group consisting of C₁₋₄alkyl, C₁₋₄ alkoxy, amino, C₁₋₄alkylamino, C₅₋₆heteroaryl and C₅₋₆heterocycloalkyl, wherein the C₅₋₆heteroaryl and C₅₋₆heterocycloalkyl of R₀ are each unsubstituted or substituted with 1 to 2 substituents independently selected from the group consisting of C₁₋₄alkyl, halo and amino;
      R₃ₐ and R_{3b} are each independently selected from the group consisting of hydrogen and C₁₋₄alkyl, or R₃ₐ and R_{3b} is taken together with the carbon to which both attached to form a cyclopropyl;
   each R₁ is independently selected from the group consisting of halo, cyano, —OR₄, —C(O)R₅, —C(O)NR₆R₇, —NR₈R₉, —NHC(O)R₁₀, —NHSO₂R₁₁, —SO₂R₁₂, C₁₋₆alkyl, phenyl, C₅₋₉heteroaryl, and C₄₋₆heterocycloalkyl, wherein
      R₄ is C₁₋₆alkyl or phenyl, wherein the C₁₋₆alkyl is unsubstituted or substituted by 1 to 3 substituents independently selected from the group consisting of halo, cyano, C₁₋₄alkyl, amino, di-C₁₋₄alkylamino, and —C(O)NH₂);
      R₅ is hydrogen, C₁₋₆alkyl or C₁₋₆alkoxy;
      R₆, R₈ and R₁₁ are each independently hydrogen or C₁₋₄alkyl;
      R₇ and R₉ are each independently selected from the group consisting of hydrogen, C₁₋₄alkyl, C₁₋₄alkoxy, and C₃₋₆cycloalkyl, wherein the C₁₋₄alkyl is unsubstituted or substituted with 1 to 3 substituents independently selected from the group consisting of amino, C₁₋₄alkyl, C₁₋₄alkylamino, di-C₁₋₄alkylamino, C₁₋₄ alkoxycarbonylamino, and C₅₋₆heterocycloalkyl;
      R₁₀ is C₁₋₆alkyl, C₁₋₆alkoxy or C₃₋₆cycloalkyl, wherein the C₁₋₆alkyl of R₁₀ is unsubstituted or substituted by 1-2 substituents independently selected from amino and C₃₋₆cycloalkyl, and the C₃₋₆cycloalkyl of R₁₀ is unsubstituted or substituted by 1 to 2 substituents independently selected from the group consisting of amino and amino-C₁₋₄alkyl,
      R₁₂ is C₁₋₄alkyl, amino or C₁₋₄alkylamino,
      the C₁₋₆alkyl of R₁ is unsubstituted or substituted with 1-3 substituents independently selected from the group consisting of halo, cyano, methoxy, amino, C₁₋₄alkylamino, C₅₋₆cycloalkyl, and phenyl; and
      the phenyl, C₅₋₆heteroaryl and C₃₋₆heterocycloalkyl of R₁ are each independently unsubstituted or substituted with 1 to 2 substituents independently selected from the group consisting of C₁₋₄alkyl, amino, C₁₋₄alkylamino, —C(O)CH₃, and benzyl;
   R₁₅ and R₁₆ are each independently hydrogen, C₁₋₄alkyl or haloC₁₋₄alkyl;
   each R₁₇ is independently selected from the group consisting of cyano, halo, oxo, OR₁₈, —C(O)R₁₉, —NR₂₀R₂₁, —SO₂R₂₂, —SO₂NHR₂₃, C₁₋₄alkyl, phenyl, C₅₋₉heteroaryl, C₃₋₆cycloalkyl and C₄₋₆heterocycloalkyl, wherein
      R₁₈ is selected from the group consisting of hydrogen, C₁₋₄alkyl, haloC₁₋₄alkyl and phenyl;
      R₁₉ is selected from the group consisting of hydrogen, C₁₋₄alkyl, amino, and C₁₋₄alkylamino;
      R₂₀, R₂₁ and R₂₂ are each independently hydrogen or C₁₋₄alkyl;
      R₂₃ is hydrogen, C₁₋₄alkyl, or C₅₋₆heteroaryl
      the C₁₋₄alkyl of R₁₇ is unsubstituted or substituted with 1-3 substituents independently selected from the group consisting of halo, C₁₋₄alkoxy and amino; and
      the phenyl, C₅₋₉heteroaryl, C₃₋₆cycloalkyl and C₄₋₆heterocycloalkyl of R₁₇ are each independently unsubstituted or substituted by 1 to 2 substituents independently selected from the group consisting of C₁₋₄alkyl, halo-C₁₋₄alkyl, C₁₋₄alkoxy-C₁₋₄alkyl, and C₁₋₄alkoxy.

In one embodiment, with reference to the embodiment above, the compound of the invention is where L is selected from the group consisting of *—CH₂N(R₂)—, *—C(O)—, *—C(O)N(R₂)—, *—C(O)N(R₂)C(R₃ₐ)(R_{3b})—, *—N(R₂)C(O)—, *—N(R₂)SO₂—, and C₁₋₆alkylene, wherein * represents the point of attachment of L to Ring B; R₂ is C₁₋₄alkyl; R₃ₐ and R_{3b} are each independently selected from the group consisting of hydrogen and C₁₋₄alkyl, or R₃ₐ and R_{3b} is taken together with the carbon to which both attached to form a cyclopropyl.

In one variation, the compounds of the invention according to any one of the above embodiments is where L is selected from the group consisting of *—(CH₂)—, *—CH₂N(CH₃)—, *—C(O)—, *—C(O)NH—, *—C(O)N(CH₃)—, *—C(O)N(CH₂CH₂OCH₃)—, *—C(O)N(CH₂CH₂NH₂)—, *—C(O)N(CH₂CH₂-tetrahydropyran-4-yl)-, *—C(O)N(CH₂CH₃)—, *—C(O)N(CH₂CH₂N(CH₃)₂)—, *—C(O)NHCH₂—, *—C(O)N(CH₃)CH₂—, *—C(O)N(CH(CH₃)₂)—, *—C(O)N(CH₃)CH(CH₃)—, *—C(O)N(CH₃)C(CH₃)₂—, *—NHCH₂—, *—N(CH₃)C(O)—, and *—N(CH₃)S(O)₂—.

In another variation, L is selected from the group consisting of *—CH₂N(CH₃)—, *—C(O)NH—, *—C(O)N(CH₃)—, *—C(O)N(CH₂CH₂OCH₃)—, *—C(O)N(CH₂CH₂N(CH₃)₂)—, *—C(O)N(CH₂CH₂NH₂)—, *—C(O)N(CH₂CH₂-tetrahydropyran-4-yl)-, *—C(O)N(CH₂CH₃)—, *—C(O)N(CH(CH₃)₂)—, *—NHCH₂—, *—N(CH₃)C(O)—, and *—N(CH₃)S(O)₂—. In still another variation, *—CH₂N(CH₃)—, *—C(O)NH—, *—C(O)N(CH₃)—, *—C(O)N(CH₂CH₂OCH₃)—, *—C(O)N(CH₂CH₂NH₂)—, *—C(O)N(CH₂CH₃)—, *—C(O)N(CH₂CH₂N(CH₃)₂)—, *—C(O)N(CH(CH₃)₂)—, and *—N(CH₃)C(O)—.

In a particular variation of the compounds of the invention according the above embodiments, L is *—C(O)N(CH₃)—. In another particular variation, L is *—CH₂N(CH₃)—. In still another particular variation, L is *—(CH₂)—. In yet another particular variation, L is *—C(O)—.

In another embodiment of the compounds of the invention according to any one of the above embodiments and variations, Ring A is selected from the group consisting of C₆₋₁₀aryl and C₅₋₁₀ heteroaryl. In one variation, Ring A is selected from the group consisting of

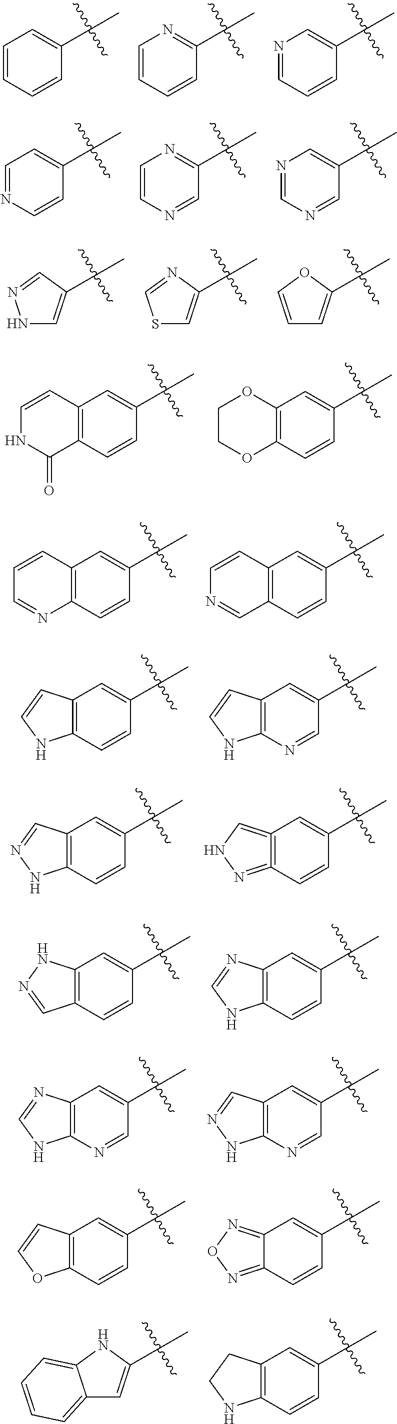

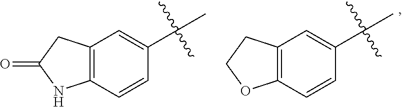 [SMILES: *c1ccc2c(c1)CC(=O)N2 ; *c1ccc2c(c1)CCO2]

each of which is unsubstituted or substituted with 1 to 2 R₁ groups.

In another variation, Ring A is selected from the group consisting of

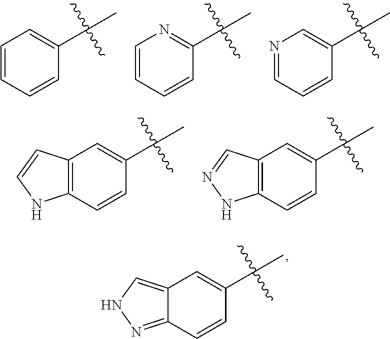 [SMILES: *c1ccccc1 ; *c1ccccn1 ; *c1cccnc1 ; *c1ccc2[nH]ccc2c1 ; *c1ccc2[nH]ncc2c1 ; *c1ccc2n[nH]cc2c1]

each of which is unsubstituted or substituted with 1 to 2 R₁ groups

In still another variation, Ring A is selected from the group consisting of phenyl and pyridinyl, each of which is unsubstituted or substituted with 1 to 2 R₁ groups. In a particular variation, Ring A is of the formula:

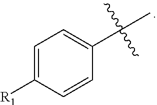

In another particular variation, Ring A is of the formula:

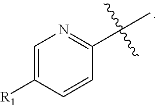

In still another particular variation, Ring A is of the formula:

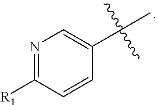

In another embodiment of the compounds of the invention according to any one of the above embodiments and variations, Ring C is selected from the group consisting of phenyl, C₅₋₁₀heteroaryl and C₅₋₁₀heterocycloalkyl. In one variation, Ring C is selected from the group consisting of

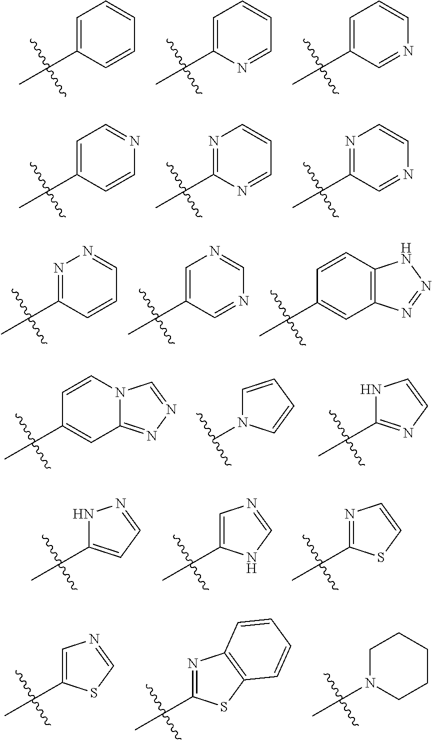

each of which is unsubstituted or substituted with 1 to 2 R₁₇ groups.

In another variation, Ring C is selected from the group consisting of phenyl and pyridyl, each of which is unsubstituted or substituted with 1 to 2 R₁₇ groups. In one particular variation, Ring C is of the formula:

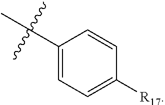

In another particular variation, Ring C is of the formula:

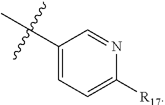

In still another particular variation, Ring C is of the formula:

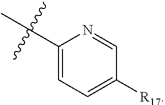

In one special variation, Ring C is C₆heterocycloalkyl or fused bicyclyl comprising a C₅₋₆heterocycloalkyl fused to a phenyl, wherein the C₆heterocycloalkyl and fused bicyclyl are each unsubstituted or substituted with 1 to 3 (R₁₇) group. Specifically, Ring C is selected from the group consisting of

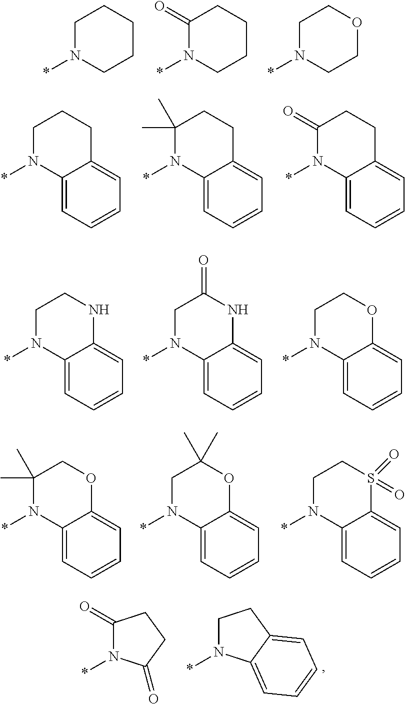

where each of the above group is either unsubstituted or substituted with 1 to 2 R₁₇ groups.

More specifically, Ring C is selected from the group consisting of

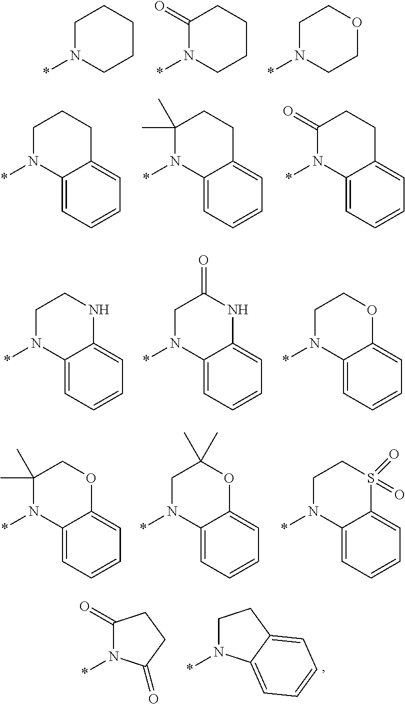

each of which is unsubstituted or substituted with 1 to 2 R₁₇ groups.

Most specifically, Ring C is selected from the group consisting of

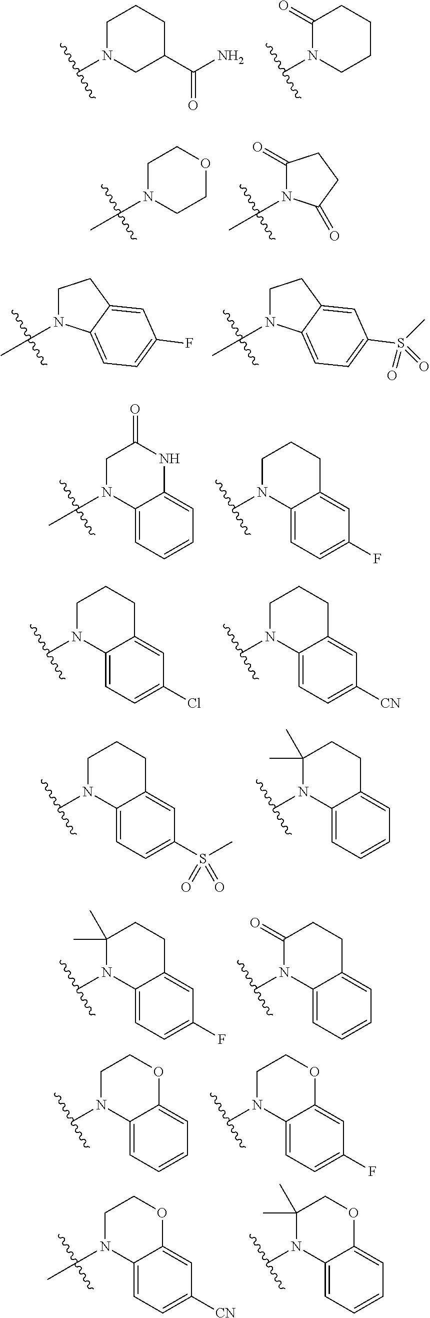

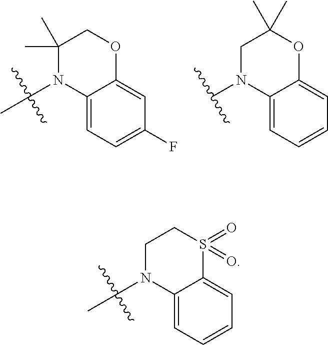 [SMILES: *N1c2ccc(F)cc2OCC1(C)C ; *N1CC(C)(C)Oc2ccccc21 ; *N1CCS(=O)(=O)c2ccccc21]

In one particular variation, Ring C is

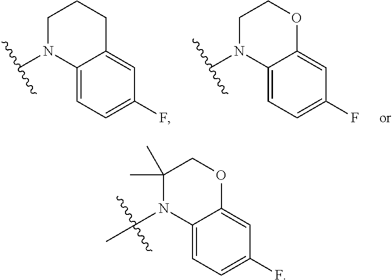

In another particular variation, Ring C is

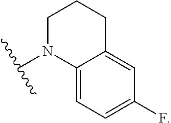 [SMILES: *N1CCCc2cc(F)ccc21]

In another embodiment of the compounds of the invention according to any one of the above embodiments and variations, each R₁ is independently selected from the group consisting of fluoro, chloro, cyano, methyl, isopropyl, t-butyl, cyanopropyl, —CH(CH₃)(OCH₃), trifluoromethyl, difluoromethyl, —CF₂CH₃, —C(CH₃)₂CN, —CH₂NH₂, —CH₂N(CH₃)₂, —CH₂-morpholinyl, methoxy, proproxy, isoproproxy, difluoromethoxy, trifluoromethoxy, —OCH₂CF₃, cyanomethoxy, 2-aminoethoxy, —O(CH₂)₂N(CH₃)₂, —OC(CH₃)₂CH₂NH₂, —OC(CH₃)₂C(O)NH₂, and phenoxy.

In one variation, each R₁ is independently selected from the group consisting of —C(O)CH₃, —C(O)OCH₃, —C(O)NH₂, —C(O)NHCH₃, —C(O)N(CH₃)₂, —C(O)N(CH₂CH₃)₂, —C(O)N(CH₃)(OCH₃), —C(O)NH((CH₂)₂N(CH₃)₂), —C(O)NH(cyclopropyl), and —C(O)NH(cyclohexyl).

In another variation, each R₁ is independently selected from the group consisting of amino, methylamino, dimethylamino, —NH(OCH₃), —NH(CH₂C(NH₂)(CH₃)₂), —NH(C(CH₃)₂CH₂NH(C(O)OC(CH₃)₃)), —NH(C(CH₃)₂CH₂NH₂), —NH((CH₂)₂-morpholinyl), —NH(C(O)CH₃), —NH(C(O)CH₂NH₂), —NH(C(O)CH(NH₂)(CH₃)), —NH(C(O)CH(NH₂)CH(CH₃)₂), —NH(C(O)C(NH₂)(CH₃)₂), —NH(C(O)CH₂CH(NH₂)(CH₃)), —NH(C(O)CH₂CH(NH₂)CH(CH₃)₂), —NH(C(O)CH(NH₂)(cyclopropyl)), —NH(C(O)(1-aminomethylcyclopropyl)), —NH(C(O)CH(NH₂)-cyclobutyl), —NH(C(O)CH(NH₂)-cyclohexyl), —NH(C(O)-2-aminocyclopentyl), —NH(C(O)OCH₃), —NH(C(O)OCH₂CH₃), —NH(C(O)OCH(CH₃)₂), —NH(SO₂CH₃), and —NH(SO₂CH(CH₃)₂).

In still another variation, each R₁ is independently selected from the group consisting of —SO₂CH₃, —SO₂CH(CH₃)₂, —SO₂NH₂, and —SO₂N(CH₃)₂. In yet still another variation, wherein each R₁ is independently selected from the group consisting of

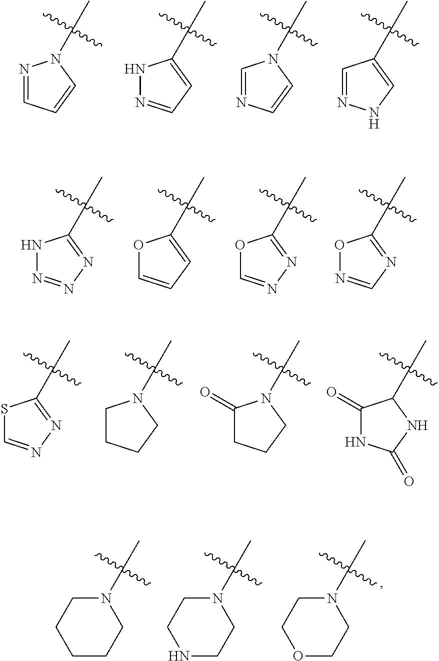

each of which is unsubstituted or substituted by 1 to 2 substituents independently selected from the group consisting of methyl, ethyl, —NH₂, —NH(CH₃), —C(O)CH₃, and benzyl.

Particularly, each R₁ is independently selected from the group consisting of

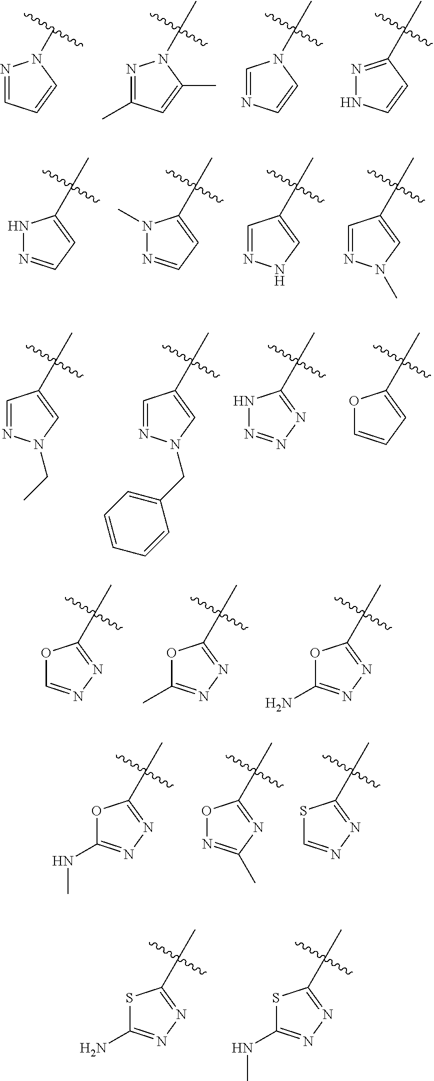

and also particularly, each R₁ is independently selected from the group consisting of

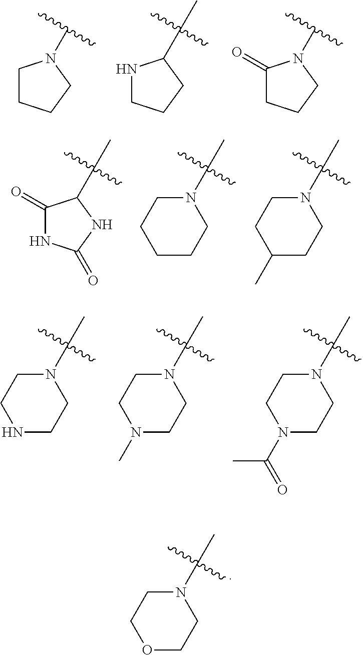

In some particular embodiment, each R₁ is independently selected from the group consisting of trifluoromethyl, —C(O)NH₂, —C(O)NHCH₃, —C(O)NH(CH₂)₂N(CH₃)₂, and —NHC(O)CH(NH₂)-cycloalkyl. In one variation, R₁ is trifluoromethyl. In another variation, R₁ is —C(O)NH₂. In still another variation, R₁ is —C(O)NH₂.

In some embodiments of the compounds of the invention according to any one of the above embodiments and variations, n is 0. In one variation, n is 1. In yet another variation, n is 2.

In some embodiments of the compounds of the invention according to any one of the above embodiments and variations, R₁₅ is hydrogen.

In some embodiments of the compounds of the invention according to any one of the above embodiments and variations, R₁₆ is hydrogen.

In some embodiments of the compounds of the invention according to any one of the above embodiments and variations, each R₁₇ is independently selected from the group consisting of fluoro, chloro, bromo, cyano, methyl, ethyl, t-butyl, trifluoromethyl, methoxymethyl, aminomethyl, methoxy, ethoxy, isopropoxy, difluoromethoxy, trifluoromethoxy, phenoxy, oxo, dimethylamino, methylsulfonyl, and aminocarbonyl. In other embodiments, each R₁₇ is independently selected from the group consisting of —C(O)CH₃, —C(O)NH₂, methylsulfonyl, —SO₂NH-thiazol-2-yl, and —SO₂NH₂. In still other embodiments, each R₁₇ is independently selected from the group consisting of

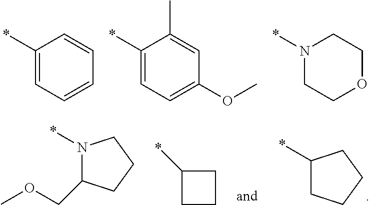

In some particular embodiments, each R₁₇ is independently selected from the group consisting of fluoro, chloro, bromo, cyano, methyl, methylsulfonyl, and aminocarbonyl. In other particular embodiments, R₁₇ is cyano. In other particular embodiments, R₁₇ is chloro. In still other particular embodiments, R₁₇ is fluoro. In yet still other embodiments, R₁₇ is methylsulfonyl.

In other embodiments of the compounds of the invention according to any one of the above embodiments and variations, p is 0. In some variations, p is 1. In other variations, p is 2.

In a special embodiment, the compounds of the invention, in reference to the first embodiment above, wherein the compound is of formula 1A:

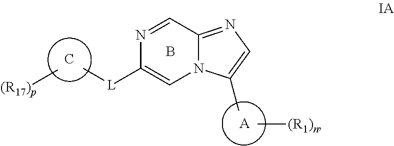

wherein
n is 1;
p is 1;
L is *—C(O)N(R₂)— or *—CH₂N(R₂)—, wherein
   * denotes the point of attachment of L to Ring B,
   R₂ is C₁₋₄alkyl or R₀—C₁₋₄alkylene, wherein R₀ is selected from the group consisting of C₁₋₄alkoxy, amino, C₁₋₄alkylamino, C₅₋₆heteroaryl and C₅₋₆heterocycloalkyl, wherein said C₅₋₆heteroaryl and C₅₋₆heterocycloalkyl are each independently unsubstituted or substituted by 1-2 substituents independently selected from the group consisting of C₁₋₄alkyl, halo, amino, and oxo;
Ring A is phenyl or pyridyl;
Ring C is phenyl or pyridyl;
each R₁ is trifluoromethyl, *—C(O)NH₂, or *—C(O)NHCH₃; and
each R₁₇ is chloro, fluoro, or cyano.

In some embodiments of the special embodiment above, L is *—C(O)N(CH₃)—. In other variation, L is *—CH₂N(CH₃)—.

In some other embodiments of the special embodiment above, Ring A is of the formula:

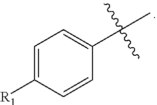

In some variations, Ring A is of the formula

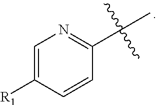

In other variations, Ring A is of the formula

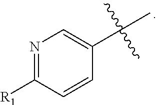

In some other embodiments of the special embodiment above, Ring C is of the formula

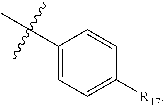

In some variations, Ring C is of the formula

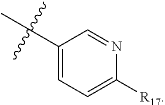

In other variations, Ring C is of the formula

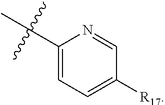

Particular examples of compounds or a pharmaceutically acceptable salt, tautomer or stereoisomer thereof, according to the present invention include, but are not limited to: N-(4-cyanophenyl)-3-(6-(3-(dimethylamino)propoxy)pyridin-3-yl)-N-methylimidazo[1,2-a]pyrazine-6-carboxamide; 4-(((3-(4-(1H-pyrazol-5-yl)phenyl)imidazo[1,2-a]pyrazin-6-yl)methyl)(methyl)amino)benzonitrile; N-((3-(4-(1H-pyrazol-5-yl)phenyl)imidazo[1,2-a]pyrazin-6-yl)methyl)-4-fluoro-N-methylaniline; 4-(((3-(4-chlorophenyl)imidazo[1,2-a]pyrazin-6-yl)methyl)(methyl)amino)benzonitrile; N-((3-(4-(1H-pyrazol-5-yl)phenyl)imidazo[1,2-a]pyrazin-6-yl)methyl)-4-chloro-N-methylaniline; N-(5-chloro-3-fluoropyridin-2-yl)-N-methyl-3-(4-(trifluoromethyl)phenyl)imidazo[1,2-a]pyrazine-6-carboxamide; 5-methanesulfonyl-1-({3-[4-(trifluoromethyl)phenyl]imidazo[1,2-a]pyrazin-6-yl}carbonyl)-2,3-dihydro-1H-indole; 4-fluoro-N-methyl-N-((3-(1-methyl-1H-indazol-5-yl)imidazo[1,2-a]pyrazin-6-yl)methyl)aniline; N-((3-(4-(1H-pyrazol-1-yl)phenyl)imidazo[1,2-a]pyrazin-6-yl)methyl)-4-chloro-N-methylaniline; N-((3-(4-(1H-pyrazol-1-yl)phenyl)imidazo[1,2-a]pyrazin-6-yl)methyl)-4-chloro-N-methylaniline; 3-(4-carbamoylphenyl)-N-(4-fluorophenyl)-N-methylimidazo[1,2-a]pyrazine-6-carboxamide; N-methyl-N-(1-methyl-1H-benzo[d][1,2,3]triazol-5-yl)-3-(4-(trifluoromethyl)phenyl)imidazo[1,2-a]pyrazine-6-carboxamide; N-(4-chlorophenyl)-N-methyl-3-[4-(1H-pyrazol-5-yl)phenyl]imidazo[1,2-a]pyrazine-6-carboxamide; N-(1,3-benzothiazol-2-yl)-N-methyl-3-[4-(trifluoromethyl)phenyl]imidazo[1,2-a]pyrazine-6-carboxamide; [4-(5-amino-1,3,4-oxadiazol-2-yl)phenyl]-N-(4-chlorophenyl)-N-methylimidazo[1,2-a]pyrazine-6-carboxamide; N-(3-methoxypyridin-2-yl)-N-methyl-3-[4-(trifluoromethyl)phenyl]imidazo[1,2-a]pyrazine-6-carboxamide; 3-(4-chlorophenyl)-N-(4-methanesulfonylphenyl)-N-methylimidazo[1,2-a]pyrazine-6-carboxamide; N-{[3-(4-chlorophenyl)imidazo[1,2-a]pyrazin-6-yl]methyl}-4-fluoro-N-methylaniline; N-(4-cyanophenyl)-N-methyl-3-(5-methylpyridin-2-yl)imidazo[1,2-a]pyrazine-6-carboxamide; N-(4-cyanophenyl)-3-(2,4-dichlorophenyl)-N-methylimidazo[1,2-a]pyrazine-6-carboxamide; N-({3-[4-(difluoromethoxy)phenyl]imidazo[1,2-a]pyrazin-6-yl}methyl)-4-fluoro-N-methylaniline; 3-(4-carbamoylphenyl)-N-(3,4-difluorophenyl)-N-methylimidazo[1,2-a]pyrazine-6-carboxamide; N-(2,4-difluorophenyl)-N-methyl-3-[4-(trifluoromethyl)phenyl]imidazo[1,2-a]pyrazine-6-carboxamide; N-methyl-4-(morpholin-4-yl)-N-{3-[4-(trifluoromethyl)phenyl]imidazo[1,2-a]pyrazin-6-yl}benzamide; 4-[({3-[4-(5-amino-1,3,4-thiadiazol-2-yl)phenyl]imidazo[1,2-a]pyrazin-6-yl}methyl)(methyl)amino]benzonitrile; 3-(4-chloro-2-methylphenyl)-N-(4-cyanophenyl)-N-methylimidazo[1,2-a]pyrazine-6-carboxamide; 3-(2-chloropyridin-4-yl)-N-(4-cyanophenyl)-N-methylimidazo[1,2-a]pyrazine-6-carboxamide; ({3-[4-(5-amino-1,3,4-thiadiazol-2-yl)phenyl]imidazo[1,2-a]pyrazin-6-yl}methyl)-6-chloro-N-methylpyridin-3-amine; N-(4-cyanophenyl)-3-(3,5-difluoropyridin-2-yl)-N-methylimidazo[1,2-a]pyrazine-6-carboxamide; N-(4-cyanophenyl)-3-(4-methoxy-3-methylphenyl)-N-methylimidazo[1,2-a]pyrazine-6-carboxamide; 3-[2-chloro-4-(trifluoromethyl)phenyl]-N-(4-cyanophenyl)-N-methylimidazo[1,2-a]pyrazine-6-carboxamide; N-(3-chloro-4-fluorophenyl)-N-methyl-3-[4-(trifluoromethyl)phenyl]imidazo[1,2-a]pyrazine-6-carboxamide; 3-acetyl-N-(6-chloropyridin-3-yl)-N-methylimidazo[1,2-a]pyrazine-6-carboxamide; 4-chloro-N-methyl-N-{[3-(1-methyl-1H-indazol-5-yl)imidazo[1,2-a]pyrazin-6-yl]methyl}aniline; N-(3-chlorophenyl)-N-methyl-3-[4-(trifluoromethyl)phenyl]imidazo[1,2-a]pyrazine-6-carboxamide; 4-(6-{[(4-fluorophenyl)(methyl)amino]methyl}imidazo[1,2-a]pyrazin-3-yl)-N,N-dimethylaniline; 4-(6-{[(4-chlorophenyl)(methyl)amino]methyl}imidazo[1,2-a]pyrazin-3-yl)-N,N-dimethylaniline; N-(4-cyanophenyl)-N-methyl-3-[4-(methylcarbamoyl)phenyl]imidazo[1,2-a]pyrazine-6-carboxamide; N-(5-ethylpyridin-2-yl)-N-methyl-3-[4-(trifluoromethyl)phenyl]imidazo[1,2-a]pyrazine-6-carboxamide; N-methyl-N-[2-(trifluoromethoxy)phenyl]-3-[4-(trifluoromethyl)phenyl]imidazo[1,2-a]pyrazine-6-carboxamide; N-(2,4-dichlorophenyl)-3-(4-methoxyphenyl)-N-methylimidazo[1,2-a]pyrazine-6-carboxamide; N-(3-chloro-4-methoxyphenyl)-N-(4-cyanophenyl)-N-methylimidazo[1,2-a]pyrazine-6-carboxamide; N-methyl-N-(4-phenoxyphenyl)-3-[4-(trifluoromethyl)phenyl]imidazo[1,2-a]pyrazine-6-carboxamide; 4-chloro-N-{[3-(4-chlorophenyl)imidazo[1,2-a]pyrazin-6-yl]methyl}-N-methylaniline; N,5-dimethyl-N-{[3-(1-methyl-1H-indazol-5-yl)imidazo[1,2-a]pyrazin-6-yl]methyl}pyridin-2-amine; N-(4-cyanophenyl)-N-methyl-3-[3-(1H-pyrazol-4-yl)phenyl]imidazo[1,2-a]pyrazine-6-carboxamide; N,5-dimethyl-N-({3-[4-(1H-pyrazol-5-yl)phenyl]imidazo[1,2-a]pyrazin-6-yl}methyl)pyridin-2-amine; N-(4-cyanophenyl)-3-(3-methoxyphenyl)-N-methylimidazo[1,2-a]pyrazine-6-carboxamide; N-(2,4-dichlorophenyl)-N-methyl-3-[4-(trifluoromethyl)phenyl]imidazo[1,2-a]pyrazine-6-carboxamide; N-[4-(difluoromethoxy)phenyl]-N-methyl-3-[4-(trifluoromethyl)phenyl]imidazo[1,2-a]pyrazine-6-carboxamide; N-methyl-N-[4-(1-methyl-1H-pyrazol-4-yl)phenyl]-3-[4-(trifluoromethyl)phenyl]imidazo[1,2-a]pyrazine-6-carboxamide; N-(3-bromo-4-fluorophenyl)-N-methyl-3-[4-(trifluoromethyl)phenyl]imidazo[1,2-a]pyrazine-6-carboxamide; N-(3,4-difluorophenyl)-N-methyl-3-(1-methyl-1H-indazol-5-yl)imidazo[1,2-a]pyrazine-6-carboxamide; N-(2-fluoropyridin-3-yl)-N-methyl-3-[4-(trifluoromethyl)phenyl]imidazo[1,2-a]pyrazine-6-carboxamide; N-(3,4-dichlorophenyl)-N-methyl-3-[4-(trifluoromethyl)phenyl]imidazo[1,2-a]pyrazine-6-carboxamide; N-(4-chloro-3-methylphenyl)-N-methyl-3-[4-(trifluoromethyl)phenyl]imidazo[1,2-a]pyrazine-6-carboxamide; N-(4-cyanophenyl)-3-[4-(difluoromethyl)phenyl]-N-methylimidazo[1,2-a]pyrazine-6-carboxamide; N-(6-bromopyridin-3-yl)-N-methyl-3-[4-(trifluoromethyl)phenyl]imidazo[1,2-a]pyrazine-6-carboxamide; N-(4-chloro-2-methoxyphenyl)-N-methyl-3-[4-(trifluoromethyl)phenyl]imidazo[1,2-a]pyrazine-6-carboxamide; 3-[4-chloro-3-(trifluoromethyl)phenyl]-N-(4-cyanophenyl)-N-methylimidazo[1,2-a]pyrazine-6-carboxamide; N-(2,4-dichlorophenyl)-N-methyl-3-(4-methylphenyl)imidazo[1,2-a]pyrazine-6-carboxamide; 4-[methyl({[3-(1-methyl-1H-indazol-5-yl)imidazo[1,2-a]pyrazin-6-yl]methyl}amino]benzonitrile; N-(5-fluoropyridin-2-yl)-N-methyl-3-[4-(trifluoromethyl)phenyl]imidazo[1,2-a]pyrazine-6-carboxamide; 4-chloro-N-({3-[4-(difluoromethoxy)phenyl]imidazo[1,2-a]pyrazin-6-yl}methyl)-N-methylaniline; N-(4-cyanophenyl)-3-(2-fluoro-4-methoxyphenyl)-N-methylimidazo[1,2-a]pyrazine-6-carboxamide; N-(5-fluoropyridin-3-yl)-N-methyl-3-[4-(trifluoromethyl)phenyl]imidazo[1,2-a]pyrazine-6-carboxamide; N-(4-cyanophenyl)-3-(4-methoxyphenyl)-N-methylimidazo[1,2-a]pyrazine-6-carboxamide; N-methyl-N-[4-(2-methyl-2H-1,2,3,4-tetrazol-5-yl)phenyl]-3-[4-(trifluoromethyl)phenyl]imidazo[1,2-a]pyrazine-6-carboxamide; N-(4-fluorophenyl)-3-(4-methoxyphenyl)-N-methylimidazo[1,2-a]pyrazine-6-carboxamide; 3-(4-chlorophenyl)-N-(2,4-dichlorophenyl)-N-methylimidazo[1,2-a]pyrazine-6-carboxamide; 3-(4-chloro-3-fluorophenyl)-N-(4-cyanophenyl)-N-methylimidazo[1,2-a]pyrazine-6-carboxamide; N-(4-cyanophenyl)-N-[2-(dimethylamino)ethyl]-3-[4-(trifluoromethyl)phenyl]imidazo[1,2-a]pyrazine-6-carboxamide; N-(2-chlorophenyl)-N-methyl-3-[4-(trifluoromethyl)phenyl]imidazo[1,2-a]pyrazine-6-carboxamide; 3-[4-(5-amino-1,3,4-thiadiazol-2-yl)phenyl]-N-(4-cyanophenyl)-N-methylimidazo[1,2-a]pyrazine-6-carboxamide; N-(4-cyanophenyl)-3-[4-(1,1-difluoroethyl)phenyl]-N-methylimidazo[1,2-a]pyrazine-6-carboxamide; N-(4-cyanophenyl)-N-methyl-3-[4-(trifluoromethyl)phenyl]imidazo[1,2-a]pyrazine-6-carboxamide; N-(4-cyanophenyl)-3-[4-(dimethylamino)phenyl]-N-methylimidazo[1,2-a]pyrazine-6-carboxamide; N-(6-chloropyridin-3-yl)-N-methyl-3-[4-(trifluoromethyl)phenyl]imidazo[1,2-a]pyrazine-6-carboxamide; N-(6-fluoropyridin-3-yl)-N-methyl-3-[4-(trifluoromethyl)phenyl]imidazo[1,2-a]pyrazine-6-carboxamide; N-(6-chloropyridin-3-yl)-N-methyl-3-(4-methylphenyl)imidazo[1,2-a]pyrazine-6-carboxamide; N-(4-chlorophenyl)-N-(6-chloropyridin-3-yl)-N-methylimidazo[1,2-a]pyrazine-6-carboxamide; N-(4-cyanophenyl)-N-methyl-3-[6-(pyrrolidin-1-yl)pyridin-3-yl]imidazo[1,2-a]pyrazine-6-carboxamide; N-(5-cyanopyridin-2-yl)-N-methyl-3-(1-methyl-1H-indazol-5-yl)imidazo[1,2-a]pyrazine-6-carboxamide; N-(6-chloropyridin-3-yl)-N-methyl-3-(1-methyl-1H-indol-5-yl)imidazo[1,2-a]pyrazine-6-carboxamide; N-(5-cyanopyridin-2-yl)-3-[4-(difluoromethoxy)phenyl]-N-methylimidazo[1,2-a]pyrazine-6-carboxamide; N-(6-fluoropyridin-2-yl)-N-methyl-3-[4-(trifluoromethyl)phenyl]imidazo[1,2-a]pyrazine-6-carboxamide; N-(4-cyanophenyl)-N-methyl-3-[4-(1H-pyrazol-1-yl)phenyl]imidazo[1,2-a]pyrazine-6-carboxamide; N-(6-methoxypyridin-3-yl)-N-methyl-3-[4-(trifluoromethyl)phenyl]imidazo[1,2-a]pyrazine-6-carboxamide; N-(5-cyanopyridin-2-yl)-N-methyl-3-[4-(trifluoromethyl)phenyl]imidazo[1,2-a]pyrazine-6-carboxamide; N-(4-cyanophenyl)-3-(6-methoxypyridin-3-yl)-N-methylimidazo[1,2-a]pyrazine-6-carboxamide; N-(5-chloropyrazin-2-yl)-N-methyl-3-[4-(trifluoromethyl)phenyl]imidazo[1,2-a]pyrazine-6-carboxamide; N-(4-cyanophenyl)-N-(propan-2-yl)-3-[4-(trifluoromethyl)phenyl]imidazo[1,2-a]pyrazine-6-carboxamide; N-(5-chloropyrimidin-2-yl)-N-methyl-3-[4-(trifluoromethyl)phenyl]imidazo[1,2-a]pyrazine-6-carboxamide; N-(6-chloropyridazin-3-yl)-N-methyl-3-[4-(trifluoromethyl)phenyl]imidazo[1,2-a]pyrazine-6-carboxamide; N-(5-cyanopyridin-2-yl)-3-[4-(difluoromethyl)phenyl]-N-methylimidazo[1,2-a]pyrazine-6-carboxamide; N-(6-bromopyridazin-3-yl)-N-methyl-3-[4-(trifluoromethyl)phenyl]imidazo[1,2-a]pyrazine-6-carboxamide; N-(5-bromopyrazin-2-yl)-N-methyl-3-[4-(trifluoromethyl)phenyl]imidazo[1,2-a]pyrazine-6-carboxamide; 3-(4-carbamoylphenyl)-N-(5-cyanopyridin-2-yl)-N-methylimidazo[1,2-a]pyrazine-6-carboxamide; N-methyl-N-(pyridin-2-yl)-3-[4-(trifluoromethyl)phenyl]imidazo[1,2-a]pyrazine-6-carboxamide; N-(6-chloropyridin-3-yl)-3-(4-methoxyphenyl)-N-methylimidazo[1,2-a]pyrazine-6-carboxamide; N-(4-fluorophenyl)-N-methyl-3-[4-(trifluoromethyl)phenyl]imidazo[1,2-a]pyrazine-6-carboxamide; N-(4-chlorophenyl)-3-[4-(difluoromethoxy)phenyl]-N-methylimidazo[1,2-a]pyrazine-6-carboxamide; 3-(4-chlorophenyl)-N-(4-fluorophenyl)-N-methylimidazo[1,2-a]pyrazine-6-carboxamide; 3-(4-carbamoylphenyl)-N-(4-cyanophenyl)-N-methylimidazo[1,2-a]pyrazine-6-carboxamide; N-(3,4-difluorophenyl)-N-methyl-3-[4-(trifluoromethyl)phenyl]imidazo[1,2-a]pyrazine-6-carboxamide; N-(4-fluorophenyl)-N-methyl-3-(1-methyl-1H-indazol-5-yl)imidazo[1,2-a]pyrazine-6-carboxamide; N-(4-fluorophenyl)-N-methyl-3-(1-methyl-1H-indazol-5-yl)imidazo[1,2-a]pyrazine-6-carboxamide; N-(4-fluorophenyl)-N-methyl-3-(4-methylphenyl)imidazo[1,2-a]pyrazine-6-carboxamide; N,3-bis(4-chlorophenyl)-N-methylimidazo[1,2-a]pyrazine-6-carboxamide; N-(2-methoxyphenyl)-N-methyl-3-[4-(trifluoromethyl)phenyl]imidazo[1,2-a]pyrazine-6-carboxamide; 6-fluoro-1-({3-[4-(trifluoromethyl)phenyl]imidazo[1,2-a]pyrazin-6-yl}carbonyl)-1,2,3,4-tetrahydroquinoline; 3-(4-chlorophenyl)-N-(3,4-difluorophenyl)-N-methylimidazo[1,2-a]pyrazine-6-carboxamide; N-(4-cyanophenyl)-N-methyl-3-(4-methylphenyl)imidazo[1,2-a]pyrazine-6-carboxamide; N-(4-cyanophenyl)-N-ethyl-3-[4-(trifluoromethyl)phenyl]imidazo[1,2-a]pyrazine-6-carboxamide; N-(4-cyanophenyl)-N-methyl-3-[4-(propan-2-yl)phenyl]imidazo[1,2-a]pyrazine-6-carboxamide; N-(4-cyanophenyl)-N-methyl-3-[4-(trifluoromethoxy)phenyl]imidazo[1,2-a]pyrazine-6-carboxamide; N-(4-cyanophenyl)-N-methyl-3-[4-(methylamino)phenyl]imidazo[1,2-a]pyrazine-6-carboxamide; 3-(4-tert-butylphenyl)-N-(4-cyanophenyl)-N-methylimidazo[1,2-a]pyrazine-6-carboxamide; N-(2-fluorophenyl)-N-methyl-3-[4-(trifluoromethyl)phenyl]imidazo[1,2-a]pyrazine-6-carboxamide; N-[4-(1-cyano-1-methylethyl)phenyl]-N-(4-cyanophenyl)-N-methylimidazo[1,2-a]pyrazine-6-carboxamide; 3-[4-(dimethylamino)phenyl]-N-(4-fluorophenyl)-N-methylimidazo[1,2-a]pyrazine-6-carboxamide; N-(4-chlorophenyl)-3-[4-(dimethylamino)phenyl]-N-methylimidazo[1,2-a]pyrazine-6-carboxamide; N-(4-cyanophenyl)-N-methyl-3-(1-methyl-1H-indol-5-yl)imidazo[1,2-a]pyrazine-6-carboxamide; 3-(4-aminophenyl)-N-(4-cyanophenyl)-N-methylimidazo[1,2-a]pyrazine-6-carboxamide; N-(4-cyanophenyl)-3-[4-(furan-2-yl)phenyl]-N-methylimidazo[1,2-a]pyrazine-6-carboxamide; N-(4-cyano-3-fluorophenyl)-N-methyl-3-[4-(trifluoromethyl)phenyl]imidazo[1,2-a]pyrazine-6-carboxamide; N-(3,4-difluorophenyl)-N-methyl-3-(1-methyl-1H-indol-5-yl)imidazo[1,2-a]pyrazine-6-carboxamide; N-(4-methanesulfonylphenyl)-3-[4-(trifluoromethyl)phenyl]imidazo[1,2-a]pyrazine-6-carboxamide; N-(4-cyanophenyl)-3-(4-methanesulfonylphenyl)-N-methylimidazo[1,2-a]pyrazine-6-carboxamide; N-(4-cyanophenyl)-3-[4-(1-methoxyethyl)phenyl]-N-methylimidazo[1,2-a]pyrazine-6-carboxamide; N-(4-methanesulfonylphenyl)-N-methyl-3-[4-(1H-pyrazol-5-yl)phenyl]imidazo[1,2-a]pyrazine-6-carboxamide; 3-(4-chlorophenyl)-N-(4-cyanophenyl)-N-methylimidazo[1,2-a]pyrazine-6-carboxamide; N-(4-cyanophenyl)-N-[2-(oxan-4-yl)ethyl]-3-[4-(trifluoromethyl)phenyl]imidazo[1,2-a]pyrazine-6-carboxamide; 3-(4-acetylphenyl)-N-(4-cyanophenyl)-N-methylimidazo[1,2-a]pyrazine-6-carboxamide; N-(3,4-difluorophenyl)-N-methyl-3-(4-methylphenyl)imidazo[1,2-a]pyrazine-6-carboxamide; N-(4-fluorophenyl)-N-methyl-3-(1-methyl-1H-indol-5-yl)imidazo[1,2-a]pyrazine-6-carboxamide; N-(3-fluorophenyl)-N-methyl-3-[4-(trifluoromethyl)phenyl]imidazo[1,2-a]pyrazine-6-carboxamide; N-(4-cyanophenyl)-N-methyl-3-[4-(2,2,2-trifluoroethoxy)phenyl]imidazo[1,2-a]pyrazine-6-carboxamide; N-(4-methoxyphenyl)-N-methyl-3-[4-(trifluoromethyl)phenyl]imidazo[1,2-a]pyrazine-6-carboxamide; N-(4-cyanophenyl)-3-[2-fluoro-4-(trifluoromethyl)phenyl]-N-methylimidazo[1,2-a]pyrazine-6-carboxamide; N-(4-chlorophenyl)-N-methyl-3-(1-methyl-1H-indol-5-yl)imidazo[1,2-a]pyrazine-6-carboxamide; 3-[4-(difluoromethoxy)phenyl]-N-(3,4-difluorophenyl)-N-methylimidazo[1,2-a]pyrazine-6-carboxamide; 3-(1H-1,3-benzodiazol-5-yl)-N-(4-cyanophenyl)-N-methylimidazo[1,2-a]pyrazine-6-carboxamide; N-(3,4-difluorophenyl)-N-methyl-3-[4-(1H-pyrazol-1-yl)phenyl]imidazo[1,2-a]pyrazine-6-carboxamide; N-(4-cyanophenyl)-3-(1H-indazol-5-yl)-N-methylimidazo[1,2-a]pyrazine-6-carboxamide; N-(6-cyanopyridin-3-yl)-N-methyl-3-[4-(trifluoromethyl)phenyl]imidazo[1,2-a]pyrazine-6-carboxamide; N-(6-cyanopyridin-3-yl)-N-methyl-3-[4-(trifluoromethyl)phenyl]imidazo[1,2-a]pyrazine-6-carboxamide; N-(4-cyanophenyl)-N-methyl-3-(2-methyl-2H-indazol-5-yl)imidazo[1,2-a]pyrazine-6-carboxamide; N-(4-cyanophenyl)-N-methyl-3-(2-methyl-2H-indazol-5-yl)imidazo[1,2-a]pyrazine-6-carboxamide; N-(4-cyanophenyl)-N-methyl-3-[2-methyl-4-(trifluoromethyl)phenyl]imidazo[1,2-a]pyrazine-6-carboxamide; N-(4-fluorophenyl)-N-methyl-3-[4-(1H-pyrazol-1-yl)phenyl]imidazo[1,2-a]pyrazine-6-carboxamide; N-(4-cyanophenyl)-N-methyl-3-[4-(propane-2-sulfonyl)phenyl]imidazo[1,2-a]pyrazine-6-carboxamide; N-(4-cyanophenyl)-3-(4-fluorophenyl)-N-methylimidazo[1,2-a]pyrazine-6-carboxamide; N-(4-cyanophenyl)-3-(1H-indazol-6-yl)-N-methylimidazo[1,2-a]pyrazine-6-carboxamide; 3-(4-chloro-3-cyanophenyl)-N-(4-cyanophenyl)-N-methylimidazo[1,2-a]pyrazine-6-carboxamide; N-(4-cyanophenyl)-N-methyl-3-[2-(propan-2-yl)-1,3-thiazol-4-yl]imidazo[1,2-a]pyrazine-6-carboxamide; N-(4-cyanophenyl)-N-methyl-3-(quinolin-6-yl)imidazo[1,2-a]pyrazine-6-carboxamide; N-(4-cyanophenyl)-3-[4-(cyclopropylcarbamoyl)phenyl]-N-methylimidazo[1,2-a]pyrazine-6-carboxamide; N-(3,4-difluorophenyl)-3-[4-(dimethylamino)phenyl]-N-methylimidazo[1,2-a]pyrazine-6-carboxamide; N-(4-cyanophenyl)-3-[4-(3,5-dimethyl-1H-pyrazol-1-yl)phenyl]-N-methylimidazo[1,2-a]pyrazine-6-carboxamide; 3-(4-carbamoylphenyl)-N-(6-chloropyridin-3-yl)-N-methylimidazo[1,2-a]pyrazine-6-carboxamide; N-(4-cyanophenyl)-N-methyl-3-[4-(propan-2-yloxy)phenyl]imidazo[1,2-a]pyrazine-6-carboxamide; N-(4-cyanophenyl)-N-methyl-3-[4-(5-methyl-1,3,4-oxadiazol-2-yl)phenyl]imidazo[1,2-a]pyrazine-6-carboxamide; 3-[4-(dimethylamino)phenyl]-N-(4-methanesulfonylphenyl)-N-methylimidazo[1,2-a]pyrazine-6-carboxamide; N-(4-cyanophenyl)-3-[4-(2,5-dioxoimidazolidin-4-yl)phenyl]-N-methylimidazo[1,2-a]pyrazine-6-carboxamide; methyl 4-{6-[(4-cyanophenyl)(methyl)carbamoyl]imidazo[1,2-a]pyrazin-3-yl}benzoate; N-(4-cyanophenyl)-3-(isoquinolin-6-yl)-N-methylimidazo[1,2-a]pyrazine-6-carboxamide; N-(4-cyanophenyl)-N-methyl-3-(1-oxo-1,2-dihydroisoquinolin-6-yl)imidazo[1,2-a]pyrazine-6-carboxamide; N-(4-cyanophenyl)-3-[3-fluoro-4-(trifluoromethyl)phenyl]-N-methylimidazo[1,2-a]pyrazine-6-carboxamide; N-(4-cyanophenyl)-N-methyl-3-(6-propoxypyridin-3-yl)imidazo[1,2-a]pyrazine-6-carboxamide; N-methyl-N-[2-(trifluoromethyl)phenyl]-3-[4-(trifluoromethyl)phenyl]imidazo[1,2-a]pyrazine-6-carboxamide; 3-(4-carbamoylphenyl)-N-(4-methanesulfonylphenyl)-N-methylimidazo[1,2-a]pyrazine-6-carboxamide; N-methyl-N-[4-(trifluoromethoxy)phenyl]-3-[4-(trifluoromethyl)phenyl]imidazo[1,2-a]pyrazine-6-carboxamide; N-methyl-N-(1-methyl-1H-imidazol-2-yl)-3-[4-(trifluoromethyl)phenyl]imidazo[1,2-a]pyrazine-6-carboxamide; N-(4-cyanophenyl)-N-methyl-3-[3-(trifluoromethyl)phenyl]imidazo[1,2-a]pyrazine-6-carboxamide; 5-fluoro-1-({3-[4-(trifluoromethyl)phenyl]imidazo[1,2-a]pyrazin-6-yl}carbonyl)-2,3-dihydro-1H-indole; N-(4-cyanophenyl)-N-methyl-3-(4-phenoxyphenyl)imidazo[1,2-a]pyrazine-6-carboxamide; 3-(1-benzyl-1H-pyrazol-4-yl)-N-(4-cyanophenyl)-N-methylimidazo[1,2-a]pyrazine-6-carboxamide; 3-[4-chloro-2-(trifluoromethyl)phenyl]-N-(4-cyanophenyl)-N-methylimidazo[1,2-a]pyrazine-6-carboxamide; N-(4-cyanophenyl)-N-methyl-3-[4-(1H-1,2,3,4-tetrazol-5-yl)phenyl]imidazo[1,2-a]pyrazine-6-carboxamide; N-(4-cyanophenyl)-N-methyl-3-[4-(pyrrolidin-1-yl)phenyl]imidazo[1,2-a]pyrazine-6-carboxamide; N-(5-cyanopyridin-2-yl)-N-methyl-3-[4-(1H-pyrazol-5-yl)phenyl]imidazo[1,2-a]pyrazine-6-carboxamide; N-(3-bromophenyl)-N-methyl-3-[4-(trifluoromethyl)phenyl]imidazo[1,2-a]pyrazine-6-carboxamide; N-(4-cyanophenyl)-N-methyl-3-(5-sulfamoylpyridin-3-yl)imidazo[1,2-a]pyrazine-6-carboxamide; N-(4-cyanophenyl)-3-{4-[methoxy(methyl)carbamoyl]phenyl}-N-methylimidazo[1,2-a]pyrazine-6-carboxamide; 3-[4-(aminomethyl)phenyl]-N-(4-cyanophenyl)-N-methylimidazo[1,2-a]pyrazine-6-carboxamide; N-(4-cyanophenyl)-N-methyl-3-[6-(trifluoromethyl)pyridin-3-yl]imidazo[1,2-a]pyrazine-6-carboxamide; N-(4-cyanophenyl)-3-[4-(diethylcarbamoyl)phenyl]-N-methylimidazo[1,2-a]pyrazine-6-carboxamide; N-(4-cyanophenyl)-N-methyl-3-(1-methyl-1H-pyrazol-4-yl)imidazo[1,2-a]pyrazine-6-carboxamide; N-methyl-N-[(6-methylpyridin-2-yl)methyl]-3-[4-(trifluoromethyl)phenyl]imidazo[1,2-a]pyrazine-6-carboxamide; 3-(3-cyanophenyl)-N-(4-cyanophenyl)-N-methylimidazo[1,2-a]pyrazine-6-carboxamide; N-[(4-fluorophenyl)methyl]-N-methyl-3-[4-(trifluoromethyl)phenyl]imidazo[1,2-a]pyrazine-6-carboxamide; N-(4-cyanophenyl)-N-methyl-3-[4-(pyrrolidin-2-yl)phenyl]imidazo[1,2-a]pyrazine-6-carboxamide; N-methyl-N-(pyridin-3-ylmethyl)-3-[4-(trifluoromethyl)phenyl]imidazo[1,2-a]pyrazine-6-carboxamide; N-(4-cyanophenyl)-N-methyl-3-(pyridin-3-yl)imidazo[1,2-a]pyrazine-6-carboxamide; 3-(5-chloropyridin-2-yl)-N-(4-cyanophenyl)-N-methylimidazo[1,2-a]pyrazine-6-carboxamide; N-(4-cyanophenyl)-3-[4-(dimethylsulfamoyl)phenyl]-N-methylimidazo[1,2-a]pyrazine-6-carboxamide; N-(4-cyanophenyl)-N-methyl-3-[2-(4-methylpiperidin-1-yl)-1,3-thiazol-4-yl]imidazo[1,2-a]pyrazine-6-carboxamide; N-(4-cyanophenyl)-N-methyl-3-(3-methylphenyl)imidazo[1,2-a]pyrazine-6-carboxamide; N-(4-cyanophenyl)-N-methyl-3-(1H-pyrazol-4-yl)imidazo[1,2-a]pyrazine-6-carboxamide; N-(4-cyanophenyl)-N-methyl-3-(1-methyl-2,3-dihydro-1H-indol-5-yl)imidazo[1,2-a]pyrazine-6-carboxamide; 3-(2,1,3-benzoxadiazol-5-yl)-N-(4-cyanophenyl)-N-methylimidazo[1,2-a]pyrazine-6-carboxamide; N-(4-cyanophenyl)-N-methyl-3-{3-methyl-3H-imidazo[4,5-b]pyridin-6-yl}imidazo[1,2-a]pyrazine-6-carboxamide; N-(4-cyanophenyl)-N-methyl-3-(2-oxo-2,3-dihydro-1H-indol-5-yl)imidazo[1,2-a]pyrazine-6-carboxamide; N-(4-methanesulfonylphenyl)-N-methyl-3-(1-methyl-1H-indazol-5-yl)imidazo[1,2-a]pyrazine-6-carboxamide; N-methyl-N-{4-[3-(trifluoromethyl)-1H-pyrazol-1-yl]phenyl}-3-[4-(trifluoromethyl)phenyl]imidazo[1,2-a]pyrazine-6-carboxamide; N-(4-cyanophenyl)-3-{4-[(dimethylamino)methyl]phenyl}-N-methylimidazo[1,2-a]pyrazine-6-carboxamide; N-(4-cyanophenyl)-N-methyl-3-[4-(1-methyl-1H-pyrazol-5-yl)phenyl]imidazo[1,2-a]pyrazine-6-carboxamide; 3-[4-(difluoromethoxy)phenyl]-N-(4-methanesulfonylphenyl)-N-methylimidazo[1,2-a]pyrazine-6-carboxamide; 4-[3-(4-carbamoylphenyl)imidazo[1,2-a]pyrazin-6-yl]benzamide; N-(4-cyanophenyl)-N-methyl-3-[5-(2-oxopyrrolidin-1-yl)pyrazin-2-yl]imidazo[1,2-a]pyrazine-6-carboxamide; 3-{4-[(2S)-2-amino-2-cyclohexylacetamido]phenyl}-N-(4-cyanophenyl)-N-methylimidazo[1,2-a]pyrazine-6-carboxamide; 4-[methyl({3-[4-(trifluoromethyl)phenyl]imidazo[1,2-a]pyrazin-6-yl}methyl)amino]benzonitrile; N-(4-cyanophenyl)-3-[4-(difluoromethoxy)phenyl]-N-methylimidazo[1,2-a]pyrazine-6-carboxamide; N-(4-bromophenyl)-N-methyl-3-[4-(trifluoromethyl)phenyl]imidazo[1,2-a]pyrazine-6-carboxamide; N-(6-chloropyridin-3-yl)-N-methyl-3-[4-(piperidin-1-yl)phenyl]imidazo[1,2-a]pyrazine-6-carboxamide; N-(4-chlorophenyl)-N-methyl-3-[4-(trifluoromethyl)phenyl]imidazo[1,2-a]pyrazine-6-carboxamide; 6-chloro-1-({3-[4-(trifluoromethyl)phenyl]imidazo[1,2-a]pyrazin-6-yl}carbonyl)-1,2,3,4-tetrahydroquinoline; N-(4-chlorophenyl)-3-(4-methoxyphenyl)-N-methylimidazo[1,2-a]pyrazine-6-carboxamide; N-(2-chloro-5-methylpyridin-3-yl)-N-methyl-3-[4-(trifluoromethyl)phenyl]imidazo[1,2-a]pyrazine-6-carboxamide; N-(3-fluoropyridin-2-yl)-N-methyl-3-[4-(trifluoromethyl)phenyl]imidazo[1,2-a]pyrazine-6-carboxamide; 4-(6-{[(4-fluorophenyl)(methyl)amino]methyl}imidazo[1,2-a]pyrazin-3-yl)benzamide; 4-(6-{[(4-cyanophenyl)(methyl)amino]methyl}imidazo[1,2-a]pyrazin-3-yl)benzamide; N-(6-chloropyridin-3-yl)-N-methyl-3-[4-(1H-pyrazol-5-yl)phenyl]imidazo[1,2-a]pyrazine-6-carboxamide; methyl N-[4-(6-{[(4-cyanophenyl)(methyl)amino]methyl}imidazo[1,2-a]pyrazin-3-yl)phenyl]carbamate N-[5-(6-{[(4-cyanophenyl)(methyl)amino]methyl}imidazo[1,2-a]pyrazin-3-yl)pyridin-2-yl]acetamide; 3-[4-(5-amino-1,3,4-oxadiazol-2-yl)phenyl]-N-(4-fluorophenyl)-N-methylimidazo[1,2-a]pyrazine-6-carboxamide; N-(4-chlorophenyl)-N-methyl-3-[4-(methylcarbamoyl)phenyl]imidazo[1,2-a]pyrazine-6-carboxamide; 4-{6-[(6-fluoro-2,2-dimethyl-1,2,3,4-tetrahydroquinolin-1-yl)carbonyl]imidazo[1,2-a]pyrazin-3-yl}benzamide; 6-fluoro-2,2-dimethyl-1-({3-[4-(trifluoromethyl)phenyl]imidazo[1,2-a]pyrazin-6-yl}carbonyl)-1,2,3,4-tetrahydroquinoline; 6-chloro-N-methyl-N-({3-[4-(trifluoromethyl)phenyl]imidazo[1,2-a]pyrazin-6-yl}methyl)pyridin-3-amine; 3-(4-cyanophenyl)-N-(4-methanesulfonylphenyl)-N-methylimidazo[1,2-a]pyrazine-6-carboxamide; 4-(6-{[methyl(5-methylpyridin-2-yl)amino]methyl}imidazo[1,2-a]pyrazin-3-yl)benzamide; 6-fluoro-1-({3-[4-(trifluoromethyl)phenyl]imidazo[1,2-a]pyrazin-6-yl}methyl)-1,2,3,4-tetrahydroquinoline; N,5-dimethyl-N-({3-[4-(trifluoromethyl)phenyl]imidazo[1,2-a]pyrazin-6-yl}methyl)pyridin-3-amine; N-(4-cyanophenyl)-3-(6-acetamidopyridin-3-yl)-N-methylimidazo[1,2-a]pyrazine-6-carboxamide; N-(6-chloropyridin-3-yl)-3-[4-(1,1-difluoroethyl)phenyl]-N-methylimidazo[1,2-a]pyrazine-6-carboxamide; N-(4-cyanophenyl)-N-(2-methoxyethyl)-3-[4-(trifluoromethyl)phenyl]imidazo[1,2-a]pyrazine-6-carboxamide; N-methyl-3-[4-(trifluoromethyl)phenyl]-N-[6-(trifluoromethyl)pyridin-3-yl]imidazo[1,2-a]pyrazine-6-carboxamide; N-(4-cyanophenyl)-N-methyl-3-[4-(1,3,4-oxadiazol-2-yl)phenyl]imidazo[1,2-a]pyrazine-6-carboxamide; N-({3-[4-(difluoromethyl)phenyl]imidazo[1,2-a]pyrazin-6-yl}methyl)-N,5-dimethylpyridin-2-amine; N-[5-(6-{[methyl(5-methylpyridin-2-yl)amino]methyl}imidazo[1,2-a]pyrazin-3-yl)pyridin-2-yl]acetamide; N-(4-cyanophenyl)-N-methyl-3-{1H-pyrrolo[2,3-b]pyridin-5-yl}imidazo[1,2-a]pyrazine-6-carboxamide; N-(4-fluorophenyl)-N-methyl-3-[4-(methylcarbamoyl)phenyl]imidazo[1,2-a]pyrazine-6-carboxamide; methyl N-(4-{6-[(6-chloropyridin-3-yl)(methyl)carbamoyl]imidazo[1,2-a]pyrazin-3-yl}phenyl)carbamate; N-(4-cyanophenyl)-N-methyl-3-[6-(methylamino)pyridin-3-yl]imidazo[1,2-a]pyrazine-6-carboxamide; 3-[4-(2-aminoacetamido)phenyl]-N-(4-cyanophenyl)-N-methylimidazo[1,2-a]pyrazine-6-carboxamide; 3-[4-(5-amino-1,3,4-oxadiazol-2-yl)phenyl]-N-(4-cyanophenyl)-N-methylimidazo[1,2-a]pyrazine-6-carboxamide; N-methyl-N-(pyridin-4-yl)-3-[4-(trifluoromethyl)phenyl]imidazo[1,2-a]pyrazine-6-carboxamide; N-(2-methoxy-5-methylpyridin-3-yl)-N-methyl-3-[4-(trifluoromethyl)phenyl]imidazo[1,2-a]pyrazine-6-carboxamide; N-methyl-N-(5-methylpyridin-3-yl)-3-[4-(trifluoromethyl)phenyl]imidazo[1,2-a]pyrazine-6-carboxamide; N-(2-methoxy-5-methylpyridin-3-yl)-N-methyl-3-[4-(trifluoromethyl)phenyl]imidazo[1,2-a]pyrazine-6-carboxamide; ethyl 6-[(6-chloropyridin-3-yl)(methyl)carbamoyl]imidazo[1,2-a]pyrazine-3-carboxylate; ethyl N-(4-{6-[(4-cyanophenyl)(methyl)carbamoyl]imidazo[1,2-a]pyrazin-3-yl}phenyl)carbamate; 3-(4-{[(1S,2R)-2-aminocyclopentane]amido}phenyl)-N-(4-cyanophenyl)-N-methylimidazo[1,2-a]pyrazine-6-carboxamide; 3-{4-[(3S)-3-amino-4-methylpentanamido]phenyl}-N-(4-cyanophenyl)-N-methylimidazo[1,2-a]pyrazine-6-carboxamide; N-(4-fluorophenyl)-N-methyl-3-[4-(1H-pyrazol-5-yl)phenyl]imidazo[1,2-a]pyrazine-6-carboxamide; N-(3,4-difluorophenyl)-N-methyl-3-[4-(1H-pyrazol-5-yl)phenyl]imidazo[1,2-a]pyrazine-6-carboxamide; 4-chloro-N-methyl-N-{3-[4-(trifluoromethyl)phenyl]imidazo[1,2-a]pyrazin-6-yl}benzamide; methyl N-(5-{6-[(4-cyanophenyl)(methyl)carbamoyl]imidazo[1,2-a]pyrazin-3-yl}pyridin-2-yl)carbamate; Propan-2-yl N-(4-{6-[(4-cyanophenyl)(methyl)carbamoyl]imidazo[1,2-a]pyrazin-3-yl}phenyl)carbamate; N-(6-chloropyridin-3-yl)-N-methyl-3-{4-[5-(methylamino)-1,3,4-thiadiazol-2-yl]phenyl}imidazo[1,2-a]pyrazine-6-carboxamide; N,4-dimethyl-N-({3-[4-(trifluoromethyl)phenyl]imidazo[1,2-a]pyrazin-6-yl}methyl)aniline; 5-chloro-N-methyl-N-{3-[4-(trifluoromethyl)phenyl]imidazo[1,2-a]pyrazin-6-yl}pyridine-2-carboxamide; N-(6-chloropyridin-3-yl)-3-[4-(difluoromethyl)phenyl]-N-methylimidazo[1,2-a]pyrazine-6-carboxamide; 3-{4-[(2S)-2-amino-3-methylbutanamido]phenyl}-N-(4-cyanophenyl)-N-methylimidazo[1,2-a]pyrazine-6-carboxamide; 6-chloro-N-methyl-N-{3-[4-(trifluoromethyl)phenyl]imidazo[1,2-a]pyrazin-6-yl}pyridine-3-carboxamide; 4-fluoro-N-methyl-N-{3-[4-(trifluoromethyl)phenyl]imidazo[1,2-a]pyrazin-6-yl}benzamide; 3-{4-[(3R)-3-aminobutanamido]phenyl}-N-(4-cyanophenyl)-N-methylimidazo[1,2-a]pyrazine-6-carboxamide; 3-[4-(2-amino-2-cyclobutylacetamido)phenyl]-N-(4-cyanophenyl)-N-methylimidazo[1,2-a]pyrazine-6-carboxamide; 3-{4-[(2S)-2-aminopropanamido]phenyl}-N-(4-cyanophenyl)-N-methylimidazo[1,2-a]pyrazine-6-carboxamide; 3-(6-aminopyridin-3-yl)-N-(4-cyanophenyl)-N-methylimidazo[1,2-a]pyrazine-6-carboxamide; 3-[4-(cyanomethoxy)phenyl]-N-(4-cyanophenyl)-N-methylimidazo[1,2-a]pyrazine-6-carboxamide; 1-({3-[4-(trifluoromethyl)phenyl]imidazo[1,2-a]pyrazin-6-yl}carbonyl)-1,2,3,4-tetrahydroquinoline-6-carbonitrile; N-(6-chloropyridin-3-yl)-3-[2-fluoro-4-(trifluoromethyl)phenyl]-N-methylimidazo[1,2-a]pyrazine-6-carboxamide; N-methyl-4-(trifluoromethyl)-N-{3-[4-(trifluoromethyl)phenyl]imidazo[1,2-a]pyrazin-6-yl}benzamide; N-methyl-N-(6-methylpyridin-3-yl)-3-[4-(trifluoromethyl)phenyl]imidazo[1,2-a]pyrazine-6-carboxamide; 3-{4-[(2-amino-2-methylpropyl)amino]phenyl}-N-(4-cyanophenyl)-N-methylimidazo[1,2-a]pyrazine-6-carboxamide; N-(6-chloropyridin-3-yl)-3-(4-acetamidophenyl)-N-methylimidazo[1,2-a]pyrazine-6-carboxamide; N-(6-chloropyridin-3-yl)-3-(2-fluoro-4-methoxyphenyl)-N-methylimidazo[1,2-a]pyrazine-6-carboxamide; 7-fluoro-4-({3-[4-(trifluoromethyl)phenyl]imidazo[1,2-a]pyrazin-6-yl}methyl)-3,4-dihydro-2H-1,4-benzoxazine; 3-{4-[(2R)-2-aminopropanamido]phenyl}-N-(4-cyanophenyl)-N-methylimidazo[1,2-a]pyrazine-6-carboxamide; 4-cyano-N-methyl-N-{3-[4-(trifluoromethyl)phenyl]imidazo[1,2-a]pyrazin-6-yl}benzamide; N-(4-cyanophenyl)-3-{4-[2-(dimethylamino)ethoxy]phenyl}-N-methylimidazo[1,2-a]pyrazine-6-carboxamide; N-(4-cyanophenyl)-N,2-dimethyl-3-[4-(trifluoromethyl)phenyl]imidazo[1,2-a]pyrazine-6-carboxamide; 3-{4-[(2R)-2-amino-3-methylbutanamido]phenyl}-N-(4-cyanophenyl)-N-methylimidazo[1,2-a]pyrazine-6-carboxamide; N,5-dimethyl-N-({3-[4-(trifluoromethyl)phenyl]imidazo[1,2-a]pyrazin-6-yl}methyl)pyridin-2-amine; N-(6-methoxy-5-methylpyridin-3-yl)-N-methyl-3-[4-(trifluoromethyl)phenyl]imidazo[1,2-a]pyrazine-6-carboxamide; N,4-dimethyl-N-{3-[4-(trifluoromethyl)phenyl]imidazo[1,2-a]pyrazin-6-yl}benzamide; N-(6-methoxypyridin-2-yl)-N-methyl-3-[4-(trifluoromethyl)phenyl]imidazo[1,2-a]pyrazine-6-carboxamide; N-(4-cyanophenyl)-3-(4-{[2-(dimethylamino)ethyl]carbamoyl}phenyl)-N-methylimidazo[1,2-a]pyrazine-6-carboxamide; 3-{4-[(2S)-2-amino-2-cyclopropylacetamido]phenyl}-N-(4-cyanophenyl)-N-methylimidazo[1,2-a]pyrazine-6-carboxamide; N-(4-cyano-2-fluorophenyl)-N-methyl-3-[4-(trifluoromethyl)phenyl]imidazo[1,2-a]pyrazine-6-carboxamide; N,4,4-trimethyl-N-{3-[4-(trifluoromethyl)phenyl]imidazo[1,2-a]pyrazin-6-yl}piperidine-1-carboxamide; 3-[6-(2-aminoacetamido)pyridin-3-yl]-N-(4-cyanophenyl)-N-methylimidazo[1,2-a]pyrazine-6-carboxamide; N-(2-methoxypyridin-4-yl)-N-methyl-3-[4-(trifluoromethyl)phenyl]imidazo[1,2-a]pyrazine-6-carboxamide; N-(4-cyanophenyl)-N-methyl-3-(6-methylpyridin-3-yl)imidazo[1,2-a]pyrazine-6-carboxamide; N-(1-ethyl-1H-pyrazol-5-yl)-N-methyl-3-[4-(trifluoromethyl)phenyl]imidazo[1,2-a]pyrazine-6-carboxamide; N-methyl-N-(2-methylpyridin-4-yl)-3-[4-(trifluoromethyl)phenyl]imidazo[1,2-a]pyrazine-6-carboxamide; tert-butyl N-{2-[(4-{6-[(4-cyanophenyl)(methyl)carbamoyl]imidazo[1,2-a]pyrazin-3-yl}phenyl)amino]-2-methylpropyl}carbamate; N-(6-chloropyridin-3-yl)-3-(1H-indol-5-yl)-N-methylimidazo[1,2-a]pyrazine-6-carboxamide; 3-(4-methoxy-2-methylphenyl)-N-[6-(4-methoxy-2-methylphenyl)pyridin-3-yl]-N-methylimidazo[1,2-a]pyrazine-6-carboxamide; N-(4-methoxypyridin-3-yl)-N-methyl-3-[4-(trifluoromethyl)phenyl]imidazo[1,2-a]pyrazine-6-carboxamide; N-(6-chloro-5-methylpyridin-3-yl)-N-methyl-3-[4-(trifluoromethyl)phenyl]imidazo[1,2-a]pyrazine-6-carboxamide; 7-fluoro-4-({3-[4-(trifluoromethyl)phenyl]imidazo[1,2-a]pyrazin-6-yl}carbonyl)-3,4-dihydro-2H-1,4-benzoxazine; -({3-[4-(trifluoromethyl)phenyl]imidazo[1,2-a]pyrazin-6-yl}carbonyl)-3,4-dihydro-2H-1,4-benzoxazine; 3-(4-{[1-(aminomethyl)cyclopropane]amido}phenyl)-N-(4-cyanophenyl)-N-methylimidazo[1,2-a]pyrazine-6-carboxamide; N-methyl-6-(trifluoromethyl)-N-{3-[4-(trifluoromethyl)phenyl]imidazo[1,2-a]pyrazin-6-yl}pyridine-3-carboxamide; N-methyl-N-(4-methylpyridin-3-yl)-3-[4-(trifluoromethyl)phenyl]imidazo[1,2-a]pyrazine-6-carboxamide; N-(4-ethoxypyridin-2-yl)-N-methyl-3-[4-(trifluoromethyl)phenyl]imidazo[1,2-a]pyrazine-6-carboxamide; 4-methoxy-N-methyl-N-{3-[4-(trifluoromethyl)phenyl]imidazo[1,2-a]pyrazin-6-yl}benzamide; N-(6-chloropyridin-3-yl)-N-methyl-3-{1H-pyrrolo[2,3-b]pyridin-5-yl}imidazo[1,2-a]pyrazine-6-carboxamide; N-methyl-N-[4-(1H-1,2,4-triazol-1-yl)phenyl]-3-[4-(trifluoromethyl)phenyl]imidazo[1,2-a]pyrazine-6-carboxamide; N-(4-cyanophenyl)-3-(4-methanesulfonamidophenyl)-N-methylimidazo[1,2-a]pyrazine-6-carboxamide; 4-({3-[4-(trifluoromethyl)phenyl]imidazo[1,2-a]pyrazin-6-yl}carbonyl)-3,4-dihydro-2H-1,4-benzoxazine-7-carbonitrile; 6-methanesulfonyl-1-({3-[4-(trifluoromethyl)phenyl]imidazo[1,2-a]pyrazin-6-yl}carbonyl)-1,2,3,4-tetrahydroquinoline; N-(5-methanesulfonylpyridin-2-yl)-N-methyl-3-[4-(trifluoromethyl)phenyl]imidazo[1,2-a]pyrazine-6-carboxamide; methyl N-[4-(6-{[methyl(5-methylpyridin-2-yl)amino]methyl}imidazo[1,2-a]pyrazin-3-yl)phenyl]carbamate; 3-(1H-indol-2-yl)-N-[6-(1H-indol-2-yl)pyridin-3-yl]-N-methylimidazo[1,2-a]pyrazine-6-carboxamide; N-(6-chloropyridin-3-yl)-N-methyl-3-(3-methyl-1H-indazol-5-yl)imidazo[1,2-a]pyrazine-6-carboxamide; N-methyl-N-[5-(morpholin-4-yl)pyridin-2-yl]-3-[4-(trifluoromethyl)phenyl]imidazo[1,2-a]pyrazine-6-carboxamide; 4-fluoro-N-methyl-N-{3-[4-(trifluoromethyl)phenyl]imidazo[1,2-a]pyrazin-6-yl}piperidine-1-carboxamide; N-(4-cyanophenyl)-3-(6-methanesulfonamidopyridin-3-yl)-N-methylimidazo[1,2-a]pyrazine-6-carboxamide; 3-{4-[(1-amino-2-methylpropan-2-yl)amino]phenyl}-N-(4-cyanophenyl)-N-methylimidazo[1,2-a]pyrazine-6-carboxamide; 3-(1-benzofuran-5-yl)-N-(6-chloropyridin-3-yl)-N-methylimidazo[1,2-a]pyrazine-6-carboxamide; N-methyl-N-(pyridin-3-yl)-3-[4-(trifluoromethyl)phenyl]imidazo[1,2-a]pyrazine-6-carboxamide; N-methyl-N-(5-methylpyrazin-2-yl)-3-[4-(trifluoromethyl)phenyl]imidazo[1,2-a]pyrazine-6-carboxamide; N-methyl-N-(5-methylpyrazin-2-yl)-3-[4-(trifluoromethyl)phenyl]imidazo[1,2-a]pyrazine-6-carboxamide; N-methyl-3-[4-(trifluoromethyl)phenyl]-N-[2-(trifluoromethyl)pyridin-4-yl]imidazo[1,2-a]pyrazine-6-carboxamide; N-(6-chloropyridin-3-yl)-N-methyl-3-[4-(1H-pyrazol-1-yl)phenyl]imidazo[1,2-a]pyrazine-6-carboxamide; 5-{6-[(4-cyanophenyl)(methyl)carbamoyl]imidazo[1,2-a]pyrazin-3-yl}-N-methylpyridine-2-carboxamide; N-(6-chloropyridin-3-yl)-3-[3-fluoro-4-(trifluoromethyl)phenyl]-N-methylimidazo[1,2-a]pyrazine-6-carboxamide; N-(4-cyanophenyl)-3-(2-methoxypyrimidin-5-yl)-N-methylimidazo[1,2-a]pyrazine-6-carboxamide; N-(4-ethylpyridin-2-yl)-3-[4-(trifluoromethyl)phenyl]imidazo[1,2-a]pyrazine-6-carboxamide; N-(4-cyanophenyl)-3-(6-methoxypyrazin-2-yl)-N-methylimidazo[1,2-a]pyrazine-6-carboxamide; N-(6-chloropyridin-3-yl)-3-(6-methoxypyridin-3-yl)-N-methylimidazo[1,2-a]pyrazine-6-carboxamide; N-methyl-3-[4-(trifluoromethyl)phenyl]-N-[5-(trifluoromethyl)pyridin-2-yl]imidazo[1,2-a]pyrazine-6-carboxamide; N-methyl-3-[4-(trifluoromethyl)phenyl]-N-[5-(trifluoromethyl)pyridin-2-yl]imidazo[1,2-a]pyrazine-6-carboxamide; N-(4-cyanophenyl)-N-methyl-3-[4-(3-methyl-1,2,4-oxadiazol-5-yl)phenyl]imidazo[1,2-a]pyrazine-6-carboxamide; N-(6-chloropyridin-3-yl)-N-methyl-3-[4-(morpholin-4-yl)phenyl]imidazo[1,2-a]pyrazine-6-carboxamide; N,3-bis(4-cyanophenyl)-N-methyl-2-(trifluoromethyl)imidazo[1,2-a]pyrazine-6-carboxamide; N-(2,6-dichloropyridin-3-yl)-N-methyl-3-[4-(trifluoromethyl)phenyl]imidazo[1,2-a]pyrazine-6-carboxamide; N-(6-chloropyridin-3-yl)-3-[4-(cyclopropylcarbamoyl)phenyl]-N-methylimidazo[1,2-a]pyrazine-6-carboxamide; N-(4-cyanophenyl)-3-[2-(dimethylamino)pyrimidin-5-yl]-N-methylimidazo[1,2-a]pyrazine-6-carboxamide; N-methyl-N-(6-methylpyridin-2-yl)-3-[4-(trifluoromethyl)phenyl]imidazo[1,2-a]pyrazine-6-carboxamide; N-[2-(difluoromethoxy)phenyl]-N-methyl-3-[4-(trifluoromethyl)phenyl]imidazo[1,2-a]pyrazine-6-carboxamide; 4-cyano-N-methyl-N-{3-[4-(trifluoromethyl)phenyl]imidazo[1,2-a]pyrazin-6-yl}benzene-1-sulfonamide; N-methyl-3-[4-(trifluoromethyl)phenyl]-N-[4-(trifluoromethyl)pyridin-2-yl]imidazo[1,2-a]pyrazine-6-carboxamide; N-(6-chloropyridin-3-yl)-3-(1H-indazol-5-yl)-N-methylimidazo[1,2-a]pyrazine-6-carboxamide; 2,2-dimethyl-4-({3-[4-(trifluoromethyl)phenyl]imidazo[1,2-a]pyrazin-6-yl}carbonyl)-3,4-dihydro-2H-1,4-benzoxazine-7-carbonitrile; N-(4-cyanophenyl)-3-(6-fluoropyridin-2-yl)-N-methylimidazo[1,2-a]pyrazine-6-carboxamide; 3-[4-(1-carbamoyl-1-methylethoxy)phenyl]-N-(4-cyanophenyl)-N-methylimidazo[1,2-a]pyrazine-6-carboxamide; N-(5-methoxypyridin-2-yl)-N-methyl-3-[4-(trifluoromethyl)phenyl]imidazo[1,2-a]pyrazine-6-carboxamide; 3-(3-chloropyridin-2-yl)-N-(4-cyanophenyl)-N-methylimidazo[1,2-a]pyrazine-6-carboxamide; N-(5-chloropyridin-3-yl)-N-methyl-3-[4-(trifluoromethyl)phenyl]imidazo[1,2-a]pyrazine-6-carboxamide; N-methyl-N-(1,3-thiazol-2-yl)-3-[4-(trifluoromethyl)phenyl]imidazo[1,2-a]pyrazine-6-carboxamide; N-(6-chloropyridin-3-yl)-3-[6-(dimethylamino)pyridin-3-yl]-N-methylimidazo[1,2-a]pyrazine-6-carboxamide; N-(4-methoxypyridin-2-yl)-N-methyl-3-[4-(trifluoromethyl)phenyl]imidazo[1,2-a]pyrazine-6-carboxamide; N-{6-[(2S)-2-(methoxymethyl)pyrrolidin-1-yl]pyridin-3-yl}-N-methyl-3-[4-(trifluoromethyl)phenyl]imidazo[1,2-a]pyrazine-6-carboxamide; N-(6-chloropyridin-3-yl)-3-(2,3-dihydro-1,4-benzodioxin-6-yl)-N-methylimidazo[1,2-a]pyrazine-6-carboxamide; 3-(4-acetamidophenyl)-N-methyl-N-(5-methylpyridin-2-yl)imidazo[1,2-a]pyrazine-6-carboxamide; N-[(4-cyanophenyl)methyl]-3-[4-(trifluoromethyl)phenyl]imidazo[1,2-a]pyrazine-6-carboxamide; N-(4-cyanophenyl)-N-methyl-3-[4-(propane-2-sulfonamido)phenyl]imidazo[1,2-a]pyrazine-6-carboxamide; N-(6-chloropyridin-3-yl)-N-methyl-3-[2-(propan-2-yl)-1,3-thiazol-4-yl]imidazo[1,2-a]pyrazine-6-carboxamide; N-methyl-N-[6-(1H-1,2,4-triazol-1-yl)pyridin-3-yl]-3-[4-(trifluoromethyl)phenyl]imidazo[1,2-a]pyrazine-6-carboxamide; N-methyl-4-(trifluoromethoxy)-N-{3-[4-(trifluoromethyl)phenyl]imidazo[1,2-a]pyrazin-6-yl}benzamide; N-(6-chloropyridin-3-yl)-N-methyl-3-[6-(5-methyl-1,3,4-oxadiazol-2-yl)pyridin-3-yl]imidazo[1,2-a]pyrazine-6-carboxamide; N-(1-ethyl-1H-pyrazol-5-yl)-3-[4-(trifluoromethyl)phenyl]imidazo[1,2-a]pyrazine-6-carboxamide; 3-(4-cyanophenyl)-N-methyl-N-[4-(trifluoromethyl)phenyl]imidazo[1,2-a]pyrazine-6-carboxamide; N-benzyl-N-methyl-3-[4-(trifluoromethyl)phenyl]imidazo[1,2-a]pyrazine-6-carboxamide; 2-C-3-[4-(trifluoromethyl)phenyl]imidazo[1,2-a]pyrazine-6-pyridine-2,5-dicarboxamide; 3-[4-(2-aminoethoxyl)phenyl]-N-(4-cyanophenyl)-N-methylimidazo[1,2-a]pyrazine-6-carboxamide; 3-(6-acetamidopyridin-3-yl)-N-methyl-N-(5-methylpyridin-2-yl)imidazo[1,2-a]pyrazine-6-carboxamide; 4-({3-[4-(trifluoromethyl)phenyl]imidazo[1,2-a]pyrazin-6-yl}carbonyl)morpholine; N-[4-(aminomethyl)phenyl]-N-methyl-3-[4-(trifluoromethyl)phenyl]imidazo[1,2-a]pyrazine-6-carboxamide; N-(4-cyanophenyl)-2-methyl-3-[4-(trifluoromethyl)phenyl]imidazo[1,2-a]pyrazine-6-carboxamide; N,3-bis(4-cyanophenyl)-N-methylimidazo[1,2-a]pyrazine-6-carboxamide; N-(4-cyanophenyl)-N-methyl-3-(2-methylpyridin-4-yl)imidazo[1,2-a]pyrazine-6-carboxamide; N-(6-chloropyridin-3-yl)-N-methyl-3-(2-oxo-2,3-dihydro-1H-indol-5-yl)imidazo[1,2-a]pyrazine-6-carboxamide; 3-(2-aminopyrimidin-5-yl)-N-(4-cyanophenyl)-N-methylimidazo[1,2-a]pyrazine-6-carboxamide; N-(6-chloropyridin-3-yl)-3-ethenyl-N-methylimidazo[1,2-a]pyrazine-6-carboxamide; N-(5-cyclobutylpyridin-2-yl)-N-methyl-3-[4-(trifluoromethyl)phenyl]imidazo[1,2-a]pyrazine-6-carboxamide; N-{[4-(morpholin-4-yl)phenyl]methyl}-3-[4-(trifluoromethyl)phenyl]imidazo[1,2-a]pyrazine-6-carboxamide; N-(6-tert-butylpyridin-3-yl)-N-methyl-3-[4-(trifluoromethyl)phenyl]imidazo[1,2-a]pyrazine-6-carboxamide; N-(4-cyanophenyl)-N-methyl-3-(pyridin-4-yl)imidazo[1,2-a]pyrazine-6-carboxamide; N-(2-aminoethyl)-N-(4-cyanophenyl)-3-[4-(trifluoromethyl)phenyl]imidazo[1,2-a]pyrazine-6-carboxamide; N-(6-chloropyridin-3-yl)-3-(1H-indol-2-yl)-N-methylimidazo[1,2-a]pyrazine-6-carboxamide; N-(6-chloropyridin-3-yl)-3-{4-[(dimethylamino)methyl]phenyl}-N-methylimidazo[1,2-a]pyrazine-6-carboxamide; N-(4-cyano-1H-imidazol-5-yl)-3-[4-(trifluoromethyl)phenyl]imidazo[1,2-a]pyrazine-6-carboxamide; N-methyl-N-(3-methylpyridin-2-yl)-3-[4-(trifluoromethyl)phenyl]imidazo[1,2-a]pyrazine-6-carboxamide; N-(4-cyanophenyl)-N-methyl-3-{1-methyl-1H-pyrazolo[3,4-b]pyridin-5-yl}imidazo[1,2-a]pyrazine-6-carboxamide; 4-({3-[4-(trifluoromethyl)phenyl]imidazo[1,2-a]pyrazin-6-yl}methyl)morpholine; 5-{6-[(4-cyanophenyl)(methyl)carbamoyl]imidazo[1,2-a]pyrazin-3-yl}pyridine-2-carboxamide; N-[6-(dimethylamino)pyridin-3-yl]-N-methyl-3-[4-(trifluoromethyl)phenyl]imidazo[1,2-a]pyrazine-6-carboxamide; N-(6-methoxypyridazin-3-yl)-N-methyl-3-[4-(trifluoromethyl)phenyl]imidazo[1,2-a]pyrazine-6-carboxamide; 3-(3-amino-1H-indazol-5-yl)-N-(4-cyanophenyl)-N-methylimidazo[1,2-a]pyrazine-6-carboxamide; 1-({3-[4-(trifluoromethyl)phenyl]imidazo[1,2-a]pyrazin-6-yl}carbonyl)piperidine-3-carboxamide; N-(6-chloropyridin-3-yl)-N-methyl-3-[4-(methylamino)phenyl]imidazo[1,2-a]pyrazine-6-carboxamide; N-(2-methoxypyrimidin-5-yl)-N-methyl-3-[4-(trifluoromethyl)phenyl]imidazo[1,2-a]pyrazine-6-carboxamide; N-(4-cyanophenyl)-N-methyl-3-(6-{[2-(morpholin-4-yl)ethyl]amino}pyridin-3-yl)imidazo[1,2-a]pyrazine-6-carboxamide; N-(4-cyanophenyl)-3-(6-cyanopyridin-3-yl)-N-methylimidazo[1,2-a]pyrazine-6-carboxamide; 3-{4-[(1-amino-2-methylpropan-2-yl)oxy]phenyl}-N-(4-cyanophenyl)-N-methylimidazo[1,2-a]pyrazine-6-carboxamide; 6-N-(6-chloropyridin-3-yl)-6-N-methyl-3-N-[4-(trifluoromethyl)phenyl]imidazo[1,2-a]pyrazine-3,6-dicarboxamide; 5-C-3-[4-(trifluoromethyl)phenyl]imidazo[1,2-a]pyrazine-6-1H-imidazole-4,5-dicarboxamide; N-[1-(4-chlorophenyl)ethyl]-N-methyl-3-[4-(trifluoromethyl)phenyl]imidazo[1,2-a]pyrazine-6-carboxamide; N-{4-[(1,3-thiazol-2-yl)sulfamoyl]phenyl}-3-[4-(trifluoromethyl)phenyl]imidazo[1,2-a]pyrazine-6-carboxamide; N-(6-chloropyridin-3-yl)-N-methyl-3-[6-(methylamino)pyridin-3-yl]imidazo[1,2-a]pyrazine-6-carboxamide; 3-[4-(2-amino-2-methylpropanamido)phenyl]-N-(4-cyanophenyl)-N-methylimidazo[1,2-a]pyrazine-6-carboxamide; 3-[4-(2-aminoethoxyl)phenyl]-N-methyl-N-(5-methylpyridin-2-yl)imidazo[1,2-a]pyrazine-6-carboxamide; N-(6-chloropyridin-3-yl)-N-methyl-3-[4-(1,3,4-oxadiazol-2-yl)phenyl]imidazo[1,2-a]pyrazine-6-carboxamide; 6-fluoro-1-({8-methyl-3-[4-(trifluoromethyl)phenyl]imidazo[1,2-a]pyrazin-6-yl}carbonyl)-1,2,3,4-tetrahydroquinoline; N-(5-cyclopentylpyridin-2-yl)-N-methyl-3-[4-(trifluoromethyl)phenyl]imidazo[1,2-a]pyrazine-6-carboxamide; N-(6-chloropyridin-3-yl)-N-methyl-3-[6-(trifluoromethyl)pyridin-3-yl]imidazo[1,2-a]pyrazine-6-carboxamide; N-[2-(4-fluorophenyl)propan-2-yl]-3-[4-(trifluoromethyl)phenyl]imidazo[1,2-a]pyrazine-6-carboxamide; methyl N-(5-{6-[methyl(5-methylpyridin-2-yl)carbamoyl]imidazo[1,2-a]pyrazin-3-yl}pyridin-2-yl)carbamate; N-methyl-N-{[1,2,4]triazolo[4,3-a]pyridin-5-yl}-3-[4-(trifluoromethyl)phenyl]imidazo[1,2-a]pyrazine-6-carboxamide; 4-tert-butyl-N-methyl-N-{3-[4-(trifluoromethyl)phenyl]imidazo[1,2-a]pyrazin-6-yl}benzamide; N-(6-chloropyridin-3-yl)-3-[3-(dimethylamino)phenyl]-N-methylimidazo[1,2-a]pyrazine-6-carboxamide; N-(6-chloropyridin-3-yl)-3-(4-methanesulfonamidophenyl)-N-methylimidazo[1,2-a]pyrazine-6-carboxamide; N-methyl-N-(2-methylpyridin-3-yl)-3-[4-(trifluoromethyl)phenyl]imidazo[1,2-a]pyrazine-6-carboxamide; N-methyl-N-(5-methylpyrimidin-2-yl)-3-[4-(trifluoromethyl)phenyl]imidazo[1,2-a]pyrazine-6-carboxamide; N-(6-chloropyridin-3-yl)-3-(4-fluorophenyl)-N-methylimidazo[1,2-a]pyrazine-6-carboxamide; N-methyl-N-(4-phenyl-1,3-thiazol-2-yl)-3-[4-(trifluoromethyl)phenyl]imidazo[1,2-a]pyrazine-6-carboxamide; cyanophenyl)-N-methyl-3-(3-methyl-1H-indazol-5-yl)imidazo[1,2-a]pyrazine-6-carboxamide; N-(4-acetylphenyl)-N-methyl-3-[4-(trifluoromethyl)phenyl]imidazo[1,2-a]pyrazine-6-carboxamide; 1-({3-[4-(trifluoromethyl)phenyl]imidazo[1,2-a]pyrazin-6-yl}methyl)piperidin-2-one; N-(4-cyanophenyl)-3-[4-(dimethylcarbamoyl)phenyl]-N-methylimidazo[1,2-a]pyrazine-6-carboxamide; N-(4-cyanophenyl)-3-(1H-indol-5-yl)-N-methylimidazo[1,2-a]pyrazine-6-carboxamide; N-(6-chloropyridin-3-yl)-3-[4-(difluoromethoxy)phenyl]-N-methylimidazo[1,2-a]pyrazine-6-carboxamide; N-(4-fluoro-2-methoxyphenyl)-N-methyl-3-[4 (trifluoromethyl)phenyl]imidazo[1,2-a]pyrazine; N-(3,4-difluorophenyl)-3-(4-methoxyphenyl)-N-methylimidazo[1,2-a]pyrazine-6-carboxamide; N-(4-chlorophenyl)-N-methyl-3-(4-methylphenyl)imidazo[1,2-a]pyrazine-6-carboxamide; 3-[4-(difluoromethoxy)phenyl]-N-(4-fluorophenyl)-N-methylimidazo[1,2-a]pyrazine-6-carboxamide; N-(5-bromopyrimidin-2-yl)-N-methyl-3-[4-(trifluoromethyl)phenyl]imidazo[1,2-a]pyrazine-6-carboxamide; N-(5-chloropyridin-2-yl)-N-methyl-3-[4-(trifluoromethyl)phenyl]imidazo[1,2-a]pyrazine-6-carboxamide; N-methyl-N-[4-(propan-2-yloxy)phenyl]-3-[4-(trifluoromethyl)phenyl]imidazo[1,2-a]pyrazine-6-carboxamide; 4-[({3-[4-(dimethylamino)phenyl]imidazo[1,2-a]pyrazin-6-yl}methyl)(methyl)amino]benzonitrile; N-(4-cyanophenyl)-3-[3-(dimethylamino)phenyl]-N-methylimidazo[1,2-a]pyrazine-6-carboxamide; 3-(6-chloropyridin-3-yl)-N-(4-cyanophenyl)-N-methylimidazo[1,2-a]pyrazine-6-carboxamide; N-(3-methoxyphenyl)-N-methyl-3-[4-(trifluoromethyl)phenyl]imidazo[1,2-a]pyrazine-6-carboxamide; N-(4-cyanophenyl)-3-(4-methoxy-2-methylphenyl)-N-methylimidazo[1,2-a]pyrazine-6-carboxamide; N-(7-chloro-1,3-benzothiazol-2-yl)-3-[4-(trifluoromethyl)phenyl]imidazo[1,2-a]pyrazine-6-carboxamide; 4-fluoro-N-methyl-N-(3-{4-[5-(methylamino)-1,3,4-thiadiazol-2-yl]phenyl}imidazo[1,2-a]pyrazin-6-yl)benzamide; 3-{6-[(6-fluoro-1,2,3,4-tetrahydroquinolin-1-yl)carbonyl]imidazo[1,2-a]pyrazin-3-yl}-N,N-dimethylaniline; N,3-bis[4-(trifluoromethyl)phenyl]imidazo[1,2-a]pyrazine-6-carboxamide; N-(4-cyanophenyl)-N-methyl-3-{4-[5-(methylamino)-1,3,4-oxadiazol-2-yl]phenyl}imidazo[1,2-a]pyrazine-6-carboxamide; 4-{6-[(7-fluoro-3,3-dimethyl-3,4-dihydro-2H-1,4-benzoxazin-4-yl)carbonyl]imidazo[1,2-a]pyrazin-3-yl}benzamide; 4-{6-[(6-fluoro-1,2,3,4-tetrahydroquinolin-1-yl)carbonyl]imidazo[1,2-a]pyrazin-3-yl}benzamide; N-(4-fluorophenyl)-N-methyl-3-[4-(1,3,4-thiadiazol-2-yl)phenyl]imidazo[1,2-a]pyrazine-6-carboxamide; 4-{6-[(6-fluoro-1,2,3,4-tetrahydroquinolin-1-yl)carbonyl]imidazo[1,2-a]pyrazin-3-yl}-N-methylbenzamide; N-(4-{6-[(7-fluoro-3,3-dimethyl-3,4-dihydro-2H-1,4-benzoxazin-4-yl)carbonyl]imidazo[1,2-a]pyrazin-3-yl}phenyl)acetamide; N-methyl-4-{6-[N-methyl(4-fluorobenzene)amido]imidazo[1,2-a]pyrazin-3-yl}benzamide; N-methyl-N-(4-sulfamoylphenyl)-3-[4-(trifluoromethyl)phenyl]imidazo[1,2-a]pyrazine-6-carboxamide; 4-{6-[(7-fluoro-3,4-dihydro-2H-1,4-benzoxazin-4-yl)carbonyl]imidazo[1,2-a]pyrazin-3-yl}benzamide; 4-{6-[N-methyl(4-fluorobenzene)amido]imidazo[1,2-a]pyrazin-3-yl}benzamide; 4-fluoro-N-methyl-N-(3-{1H-pyrrolo[2,3-b]pyridin-5-yl}imidazo[1,2-a]pyrazin-6-yl)benzamide; 1-{[3-(2,3-dihydro-1-benzofuran-5-yl)imidazo[1,2-a]pyrazin-6-yl]carbonyl}-6-fluoro-1,2,3,4-tetrahydroquinoline; 3-(4-carbamoylphenyl)-N-methyl-N-(5-methylpyridin-2-yl)imidazo[1,2-a]pyrazine-6-carboxamide; 4-{6-[(7-fluoro-3,4-dihydro-2H-1,4-benzoxazin-4-yl)carbonyl]imidazo[1,2-a]pyrazin-3-yl}-N-methylbenzamide; 6-fluoro-1-{[3-(3-methanesulfonylphenyl)imidazo[1,2-a]pyrazin-6-yl]carbonyl}-1,2,3,4-tetrahydroquinoline; 3-[6-(4-acetylpiperazin-1-yl)pyridin-3-yl]-N-(4-cyanophenyl)-N-methylimidazo[1,2-a]pyrazine-6-carboxamide; 6-fluoro-1-{[3-(4-methanesulfonylphenyl)imidazo[1,2-a]pyrazin-6-yl]carbonyl}-1,2,3,4-tetrahydroquinoline; 1-{[3-(1-ethyl-1H-pyrazol-4-yl)imidazo[1,2-a]pyrazin-6-yl]carbonyl}-6-fluoro-1,2,3,4-tetrahydroquinoline; N-(4-cyanophenyl)-N-methyl-3-[6-(piperazin-1-yl)pyridin-3-yl]imidazo[1,2-a]pyrazine-6-carboxamide; N-(4-cyanophenyl)-3-(furan-2-yl)-N-methylimidazo[1,2-a]pyrazine-6-carboxamide; N-(4-cyanophenyl)-3-(furan-2-yl)-N-methylimidazo[1,2-a]pyrazine-6-carboxamide; 6-fluoro-1-({3-[2-(piperazin-1-yl)pyridin-4-yl]imidazo[1,2-a]pyrazin-6-yl}carbonyl)-1,2,3,4-tetrahydroquinoline; N-(4-chlorophenyl)-N-methyl-3-[4-(1,3,4-thiadiazol-2-yl)phenyl]imidazo[1,2-a]pyrazine-6-carboxamide; N-methyl-3-[4-(methylcarbamoyl)phenyl]-N-(5-methylpyridin-2-yl)imidazo[1,2-a]pyrazine-6-carboxamide; N-cyclohexyl-4-{6-[(6-fluoro-1,2,3,4-tetrahydroquinolin-1-yl)carbonyl]imidazo[1,2-a]pyrazin-3-yl}benzamide; 4-fluoro-N-methyl-N-[3-(1-methyl-1H-indazol-5-yl)imidazo[1,2-a]pyrazin-6-yl]benzamide; 1-[4-(5-{6-[(6-fluoro-1,2,3,4-tetrahydroquinolin-1-yl)carbonyl]imidazo[1,2-a]pyrazin-3-yl}pyridin-2-yl)piperazin-1-yl]ethan-1-one; 6-fluoro-1-({3-[3-(morpholin-4-ylmethyl)phenyl]imidazo[1,2-a]pyrazin-6-yl}carbonyl)-1,2,3,4-tetrahydroquinoline; N-(5-methylpyridin-2-yl)-3-[4-(trifluoromethyl)phenyl]imidazo[1,2-a]pyrazine-6-carboxamide; N-(4-ethylpyridin-2-yl)-N-methyl-3-[4-(trifluoromethyl)phenyl]imidazo[1,2-a]pyrazine-6-carboxamide; N-(4-cyanophenyl)-N-methyl-3-{4-[5-(methylamino)-1,3,4-thiadiazol-2-yl]phenyl}imidazo[1,2-a]pyrazine-6-carboxamide; 4-(6-{[(4-chlorophenyl)(methyl)amino]methyl}imidazo[1,2-a]pyrazin-3-yl)benzamide; N-(4-fluorophenyl)-N-methyl-3-{4-[5-(methylamino)-1,3,4-oxadiazol-2-yl]phenyl}imidazo[1,2-a]pyrazine-6-carboxamide; 4-[({3-[4-(difluoromethoxy)phenyl]imidazo[1,2-a]pyrazin-6-yl}methyl)(methyl)amino]benzonitrile; N-(4-chlorophenyl)-N-methyl-3-(1-methyl-1H-indazol-5-yl)imidazo[1,2-a]pyrazine-6-carboxamide; N-(4-cyanophenyl)-N-methyl-3-[4-(1H-pyrazol-5-yl)phenyl]imidazo[1,2-a]pyrazine-6-carboxamide; methyl N-(4-{6-[(4-cyanophenyl)(methyl)carbamoyl]imidazo[1,2-a]pyrazin-3-yl}phenyl)carbamate; N-(4-chlorophenyl)-N-methyl-3-{4-[5-(methylamino)-1,3,4-oxadiazol-2-yl]phenyl}imidazo[1,2-a]pyrazine-6-carboxamide; N-(4-cyanophenyl)-N-methyl-3-[4-(1H-pyrazol-3-yl)phenyl]imidazo[1,2-a]pyrazine-6-carboxamide; N-(4-cyanophenyl)-3-(4-acetamidophenyl)-N-methylimidazo[1,2-a]pyrazine-6-carboxamide; 3-(4-carbamoylphenyl)-N-(4-chlorophenyl)-N-methylimidazo[1,2-a]pyrazine-6-carboxamide; N-(4-cyanophenyl)-N-methyl-3-(1-methyl-1H-indazol-5-yl)imidazo[1,2-a]pyrazine-6-carboxamide; N-[3-(6-acetamidopyridin-3-yl)imidazo[1,2-a]pyrazin-6-yl]-4-fluoro-N-methylbenzamide; N-(6-chloropyridin-3-yl)-N-methyl-3-(1-methyl-1H-indazol-5-yl)imidazo[1,2-a]pyrazine-6-carboxamide; N-(4-fluoropyridin-2-yl)-N-methyl-3-[4-(trifluoromethyl)phenyl]imidazo[1,2-a]pyrazine-6-carboxamide; N-methyl-N-(5-methylpyridin-2-yl)-3-[4-(trifluoromethyl)phenyl]imidazo[1,2-a]pyrazine-6-carboxamide; N-(2-methoxypyridin-3-yl)-N-methyl-3-[4-(trifluoromethyl)phenyl]imidazo[1,2-a]pyrazine-6-carboxamide; N-(4-cyanophenyl)-N-methyl-3-[4-(1,3,4-thiadiazol-2-yl)phenyl]imidazo[1,2-a]pyrazine-6-carboxamide; (1,1-dioxido-2H-benzo[b][1,4]thiazin-4(3H)-yl)(3-(4-(trifluoromethyl)phenyl)imidazo[1,2-a]pyrazin-6-yl)methanone; 4-{6-[N-methyl(4-cyanobenzene)amido]imidazo[1,2-a]pyrazin-3-yl}benzamide; N-(2-chloro-1,3-thiazol-5-yl)-N-methyl-3-[4 (trifluoromethyl)phenyl]imidazo[1,2-a]pyrazine-6-carboxamide; 4-({3-[4-(trifluoromethyl)phenyl]imidazo[1,2-a]pyrazin-6-yl}carbonyl)-1,2,3,4-tetrahydroquinoxalin-2-one; N-methyl-N-{3-[4-(trifluoromethyl)phenyl]imidazo[1,2-a]pyrazin-6-yl}pyrimidine-5-carboxamide; N-methyl-N-(6-phenylpyridin-3-yl)-3-[4-(trifluoromethyl)phenyl]imidazo[1,2-a]pyrazine-6-carboxamide; N-methyl-N,3-bis[4-(trifluoromethyl)phenyl]imidazo[1,2-a]pyrazine-6-carboxamide; N-(4-methanesulfonylphenyl)-N-methyl-3-[4-(trifluoromethyl)phenyl]imidazo[1,2-a]pyrazine-6-carboxamide; N-(3-chloro-4-cyanophenyl)-N-methyl-3-[4-(trifluoromethyl)phenyl]imidazo[1,2-a]pyrazine-6-carboxamide; and N-(4-cyano-2-methoxyphenyl)-N-methyl-3-[4-(trifluoromethyl)phenyl]imidazo[1,2-a]pyrazine-6-carboxamide.

Particular examples of compounds or a pharmaceutically acceptable salt, tautomer or stereoisomer thereof, according to the present invention include, but are not limited to: 3-(4-carbamoylphenyl)-N-(4-fluorophenyl)-N-methylimidazo[1,2-a]pyrazine-6-carboxamide; N-(4-cyanophenyl)-N-methyl-3-[4-(methylcarbamoyl)phenyl]imidazo[1,2-a]pyrazine-6-carboxamide; N-(4-cyanophenyl)-N-methyl-3-[4-(trifluoromethyl)phenyl]imidazo[1,2-a]pyrazine-6-carboxamide; 4-[methyl({3-[4-(trifluoromethyl)phenyl]imidazo[1,2-a]pyrazin-6-yl}methyl)amino]benzonitrile; 4-(6-{[(4-fluorophenyl)(methyl)amino]methyl}imidazo[1,2-a]pyrazin-3-yl)benzamide; 4-(6-{[(4-cyanophenyl)(methyl)amino]methyl}imidazo[1,2-a]pyrazin-3-yl)benzamide; N-(4-chlorophenyl)-N-methyl-3-[4-(methylcarbamoyl)phenyl]imidazo[1,2-a]pyrazine-6-carboxamide; N-(4-cyanophenyl)-3-(6-acetamidopyridin-3-yl)-N-methylimidazo[1,2-a]pyrazine-6-carboxamide; 4-(6-{[(4-chlorophenyl)(methyl)amino]methyl}imidazo[1,2-a]pyrazin-3-yl)benzamide; methyl N-(4-{6-[(4-cyanophenyl)(methyl)carbamoyl]imidazo[1,2-a]pyrazin-3-yl}phenyl)carbamate; 3-(4-carbamoylphenyl)-N-(4-chlorophenyl)-N-methylimidazo[1,2-a]pyrazine-6-carboxamide; and N-(4-cyanophenyl)-N-methyl-3-(1-methyl-1H-indazol-5-yl)imidazo[1,2-a]pyrazine-6-carboxamide.

It is noted that the compounds of the present invention may be in the form of a pharmaceutically acceptable salt. It is further note that the compounds of the present invention may be a mixture of stereoisomers, or the compound may comprise a single stereoisomer.

Further compounds of the invention are detailed in the Examples, infra.

In another aspect, the present invention is directed to a pharmaceutical composition which includes as an active ingredient a compound according to any one of the above embodiments and variations in combination with a pharmaceutically acceptable carrier, diluent or excipient.

In one embodiment, the pharmaceutical composition further includes a second agent. The second agent may be, but not limited to, a kinase inhibitor, an anti-malarial drug or an anti-inflammatory agent.

In another embodiment, the pharmaceutical composition includes an antimalarial drug as a second agent; the selections of the antimalarial includes, but are not limited to, artemisinin, artemether, artesunate, arteflene, dihydroartemisinin, chlorproguanil, trimethoprim, chloroquine, quinine, mefloquine, amodiaquine, atovaquone, proguanil, lumefantrine, piperaquine, pyronaridine, halofantrine, pyrimethamine-sulfadoxine, quinacrine, pyrimethamine-dapsone, quinidine, amopyroquine, sulphonamides, primaquine, ferroquine, tafenoquine, arterolane, and pyronaridine.

In another embodiment, the pharmaceutical composition is a solid formulation adapted for oral administration. In another embodiment, the composition is a liquid formulation adapted for oral administration. In yet another embodiment, the composition is a tablet. In still another embodiment, the composition is a liquid formulation adapted for parenteral administration.

In yet another embodiment, the pharmaceutical composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, and intrathecally.

In another aspect, the present application is directed to a compound or a pharmaceutical composition according to any one of the above embodiments and variations for use in a therapeutic application.

In another aspect, the present application is directed to a compound or a pharmaceutical composition according to any one of the above embodiments and variations for use as a medicament.

In yet another aspect, the present invention is directed to a method for treating, preventing, inhibiting, ameliorating, or eradicating the pathology and/or symptomology of a disease caused by a Plasmodium parasite. The method involves administering to a subject a therapeutically effective amount of a compound or a pharmaceutical composition according to the above embodiments and variations. In addition, the administering can be in combination with a second agent.

In one embodiment of the method of the invention, the disease being treated is malaria. The malaria can be caused by the parasite Plasmodium falciparum, Plasmodium vivax, Plasmodium ovale, or Plasmodium malaria; in particular, the parasit Plasmodium falciparum. Further, the Plasmodium parasite can be at the blood stages; or the Plasmodium parasite can be at the hepatic stages.

In one embodiment of the method of the invention, the compounds or pharmaceutical compositions, according to the above embodiments and variations, can be administered prior to, simultaneously with, or after a second agent. The second agent can be a kinase inhibitor, an anti-malarial drug or an anti-inflammatory agent. In one particular variation of the method, the second agent is an anti-malarial drug, which includes, but is not limited to, artemisinin, artemether, artesunate, arteflene, dihydroartemisinin, chlorproguanil, trimethoprim, chloroquine, quinine, mefloquine, amodiaquine, atovaquone, proguanil, lumefantrine, piperaquine, pyronaridine, halofantrine, pyrimethamine-sulfadoxine, quinacrine, pyrimethamine-dapsone, quinidine, amopyroquine, sulphonamides, primaquine, ferroquine, tafenoquine, arterolane, and pyronaridine.

In another aspect, the invention is directed to a compound, salt, stereoisomer, or pharmaceutical composition thereof, according to any one of the above embodiments or variation, for treating, preventing, inhibiting, ameliorating, or eradicating the pathology and/or symptomology of a disease caused by a Plasmodium parasite. In one embodiment, the disease caused by the Plasmodium parasite is malaria. The Plasmodium parasite which causes the malaria can be Plasmodium falciparum, Plasmodium vivax, Plasmodium ovale, or Plasmodium malaria; in particular the parasite Plasmodium falciparum. Further, the Plasmodium parasite can be at the blood stages, or the Plasmodium parasite can be at the hepatic stages.

The above compound of invention may be administered prior to, simultaneously with, or after a second agent. The second agent may be a kinase inhibitor, an anti-malarial drug or an anti-inflammatory agent. In one particular embodiment, the second agent is an anti-malarial drug.

The antimalarial drug can be, but are not limited to, artemisinin, artemether, artesunate, arteflene, dihydroartemisinin, chlorproguanil, trimethoprim, chloroquine, quinine, mefloquine, amodiaquine, atovaquone, proguanil, lumefantrine, piperaquine, pyronaridine, halofantrine, pyrimethamine-sulfadoxine, quinacrine, pyrimethamine-dapsone, quinidine, amopyroquine, sulphonamides, primaquine, ferroquine, tafenoquine, arterolane, and pyronaridine.

In still another aspect, the present invention is directed to the use of a compound, or a salt, a stereoisomer, or a pharmaceutical composition thereof, according to the above embodiments or variations in the manufacture of a medicament for treating, preventing, inhibiting, or ameliorating the pathology and/or symptomology of a disease caused by a Plasmodium parasite. In one embodiment, the disease is malaria, and the parasite is Plasmodium falciparum, Plasmodium vivax, Plasmodium ovale, or Plasmodium malaria; in particular, the parasite is Plasmodium falciparum. Further, the Plasmodium parasite can be at the blood stages or the Plasmodium parasite can be at the hepatic stages.

The medicament may include, in addition to the compound of the invention, a second agent. The second agent can be a kinase inhibitor, an anti-malarial drug or an anti-inflammatory agent. In one particular embodiment, the second agent is an anti-malarial drug, and the drug is selected from artemisinin, artemether, artesunate, arteflene, dihydroartemisinin, chlorproguanil, trimethoprim, chloroquine, quinine, mefloquine, amodiaquine, atovaquone, proguanil, lumefantrine, piperaquine, pyronaridine, halofantrine, pyrimethamine-sulfadoxine, quinacrine, pyrimethamine-dapsone, quinidine, amopyroquine, sulphonamides, primaquine, ferroquine, tafenoquine, arterolane, and pyronaridine.

In another aspect, the invention is related to a kit which comprises a compound of any one of the above embodiments and variations, and optionally a second therapeutic agent. In one particular variation, the kit comprises the compound in a multiple dose form.

### Enumerated Embodiments

Various enumerated embodiments of the invention are described herein. It will be recognized that features specified in each embodiment may be combined with other specified features to provide further embodiments of the present invention.

In a first embodiment, the invention provides a compound of formula (I), or a pharmaceutical acceptable salt, tautomer or stereoisomer thereof,

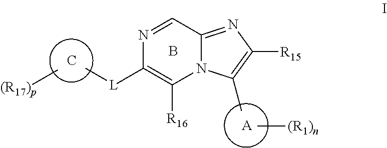

or a pharmaceutical acceptable salt, tautomer or stereoisomer thereof, wherein
n is 0, 1 or 2;
p is 0, 1, 2 or 3;
Ring A is selected from the group consisting of C₆₋₁₀aryl, C₅₋₁₀heteroaryl and fused bicyclyls comprising a C₅₋₆heterocycloalkyl fused to a phenyl;
Ring B represents the imidazo[1,2-a]pyrazine fused ring depicted in Formula 1;
Ring C is selected from the group consisting of phenyl, C₅₋₁₀heteroaryl, C₅₋₆cycloalkyl, C₅₋₆heterocycloalkyl, and fused bicyclyl comprising a C₅₋₆heterocycloalkyl fused to a phenyl;
L is selected from the group consisting of *—CH₂N(R₂)—, *—C(O)—, *—C(O)N(R₂)—, *—C(O)N(R₂)C(R₃ₐ)(R_{3b})—, *—N(R₂)C(R₃ₐ)(R_{3b})—, *—N(R₂)C(O)—, *—N(R₂)SO₂—, and C₁₋₆alkylene, wherein
   * represents the point of attachment of L to Ring B;
   R₂ is selected from the group consisting of hydrogen, C₁₋₄alkyl and R₀—C₁₋₄alkylene, wherein R₀ is selected from the group consisting of C₁₋₄alkyl, C₁₋₄alkoxy, amino, C₁₋₄alkylamino, C₅₋₆heteroaryl and C₅₋₆heterocycloalkyl, wherein the C₅₋₆heteroaryl and C₅₋₆heterocycloalkyl of R₃ are each unsubstituted or substituted with 1 to 2 substituents independently selected from the group consisting of C₁₋₄alkyl, halo and amino;
   R₃ₐ and R_{3b} are each independently selected from the group consisting of hydrogen and C₁₋₄alkyl, or R₃ₐ and R_{3b} is taken together with the carbon to which both attached to form a cyclopropyl;
each R₁ is independently selected from the group consisting of halo, cyano, —OR₄, —C(O)R₅, —C(O)NR₆R₇, —NR₈R₉, —NHC(O)R₁₀, —NHSO₂R₁₁, —SO₂R₁₂, C₁₋₆alkyl, phenyl, C₅₋₉heteroaryl, and C₄₋₆heterocycloalkyl, wherein
   R₄ is C₁₋₆alkyl or phenyl, wherein the C₁₋₆alkyl is unsubstituted or substituted by 1 to 3 substituents independently selected from the group consisting of halo, cyano, C₁₋₄alkyl, amino, di-C₁₋₄alkylamino, and —C(O)NH₂);
   R₅ is hydrogen, C₁₋₆alkyl or C₁₋₆alkoxy;
   R₆, R₈ and R₁₁ are each independently hydrogen or C₁₋₄alkyl;
   R₇ and R₉ are each independently selected from the group consisting of hydrogen, C₁₋₄alkyl, C₁₋₄alkoxy, and C₃₋₆cycloalkyl, wherein the C₁₋₄alkyl is unsubstituted or substituted with 1 to 3 substituents independently selected from the group consisting of amino, C₁₋₄alkyl, C₁₋₄alkylamino, di-C₁₋₄alkylamino, C₁₋₄alkoxycarbonylamino, and C₅₋₆heterocycloalkyl;
   R₁₀ is C₁₋₆alkyl, C₁₋₆alkoxy or C₃₋₆cycloalkyl, wherein the C₁₋₆alkyl of R₁₀ is unsubstituted or substituted by 1-2 substituents independently selected from amino and C₃₋₆cycloalkyl, and the C₃₋₆cycloalkyl of R₁₀ is unsubstituted or substituted by 1 to 2 substituents independently selected from the group consisting of amino and amino-C₁₋₄alkyl,
   R₁₂ is C₁₋₄alkyl, amino or C₁₋₄alkylamino,
   the C₁₋₆alkyl of R₁ is unsubstituted or substituted with 1-3 substituents independently selected from the group consisting of halo, cyano, methoxy, amino, C₁₋₄alkylamino, C₅₋₆cycloalkyl, and phenyl; and
   the phenyl, C₅₋₆heteroaryl and C₃₋₆heterocycloalkyl of R₁ are each independently unsubstituted or substituted with 1 to 2 substituents independently selected from the group consisting of C₁₋₄alkyl, amino, C₁₋₄alkylamino, —C(O)CH₃, and benzyl;
R₁₅ and R₁₆ are each independently hydrogen, C₁₋₄alkyl or haloC₁₋₄alkyl;
each R₁₇ is independently selected from the group consisting of cyano, halo, oxo, OR₁₈, —C(O)R₁₉, —NR₂₀R₂₁, —SO₂R₂₂, —SO₂NHR₂₃, C₁₋₄alkyl, phenyl, C₅₋₉heteroaryl, C₃₋₆cycloalkyl and C₄₋₆heterocycloalkyl, wherein
   R₁₈ is selected from the group consisting of hydrogen, C₁₋₄alkyl, haloC₁₋₄alkyl and phenyl;
   R₁₉ is selected from the group consisting of hydrogen, C₁₋₄alkyl, amino, and C₁₋₄alkylamino;
   R₂₀, R₂₁ and R₂₂ are each independently hydrogen or C₁₋₄alkyl;
   R₂₃ is hydrogen, C₁₋₄alkyl, or C₅₋₆heteroaryl
   the C₁₋₄alkyl of R₁₇ is unsubstituted or substituted with 1-3 substituents independently selected from the group consisting of halo, C₁₋₄alkoxy and amino; and
   the phenyl, C₅₋₉heteroaryl, C₃₋₆cycloalkyl and C₄₋₆heterocycloalkyl of R₁₇ are each independently unsubstituted or substituted by 1 to 2 substituents independently selected from the group consisting of C₁₋₄alkyl, halo-C₁₋₄alkyl, C₁₋₄alkoxy-C₁₋₄alkyl, and C₁₋₄alkoxy.

### Embodiment 2

A compound, or a salt, tautomer or stereoisomer thereof, according to embodiment 1, wherein L is selected from the group consisting of *—CH₂N(R₂)—, *—C(O)—, *—C(O)N(R₂)—, *—C(O)N(R₂)C(R₃ₐ)(R_{3b})—, *—N(R₂)C(O)—, *—N(R₂)SO₂—, and C₁₋₆alkylene, wherein
* represents the point of attachment of L to Ring B;
R₂ is C₁₋₄alkyl;
R₃ₐ and R_{3b} are each independently selected from the group consisting of hydrogen and C₁₋₄alkyl, or R₃ₐ and R_{3b} is taken together with the carbon to which both attached to form a cyclopropyl.

### Embodiment 3

A compound, or a salt, tautomer or stereoisomer thereof, according to embodiment 1, wherein L is selected from the group consisting of *—(CH₂)—, *—CH₂N(CH₃)—, *—C(O)—, *—C(O)NH—, *—C(O)N(CH₃)—, *—C(O)N(CH₂CH₂OCH₃)—, *—C(O)N(CH₂CH₂NH₂)—, *—C(O)N(CH₂CH₂-tetrahydropyran-4-yl)-, *—C(O)N(CH₂CH₃)—, *—C(O)N(CH₂CH₂N(CH₃)₂)—, *—C(O)NHCH₂—, *—C(O)N(CH₃)CH₂—, *—C(O)N(CH(CH₃)₂)—, *—C(O)N(CH₃)CH(CH₃)—, *—C(O)N(CH₃)C(CH₃)₂—, *—NHCH₂—, *—N(CH₃)C(O)—, and *—N(CH₃)S(O)₂—.

### Embodiment 4

A compound, or a salt, tautomer or stereoisomer thereof, according to embodiment 1, wherein L is selected from the group consisting of *—CH₂N(CH₃)—, *—C(O)NH—, *—C(O)N(CH₃)—, *—C(O)N(CH₂CH₂OCH₃)—, *—C(O)N(CH₂CH₂N(CH₃)₂)—, *—C(O)N(CH₂CH₂NH₂)—, *—C(O)N(CH₂CH₂-tetrahydropyran-4-yl)-, *—C(O)N(CH₂CH₃)—, *—C(O)N(CH(CH₃)₂)—, *—NHCH₂—, *—N(CH₃)C(O)—, and *—N(CH₃)S(O)₂—.

### Embodiment 5

A compound, or a salt, tautomer or stereoisomer thereof, according to embodiment 1, wherein L is selected from the group consisting of *—CH₂N(CH₃)—, *—C(O)NH—, *—C(O)N(CH₃)—, *—C(O)N(CH₂CH₂OCH₃)—, *—C(O)N(CH₂CH₂NH₂)—, *—C(O)N(CH₂CH₃)—, *—C(O)N(CH₂CH₂N(CH₃)₂)—, *—C(O)N(CH(CH₃)₂)—, and *—N(CH₃)C(O)—.

### Embodiment 6

A compound, or a salt, tautomer or stereoisomer thereof, according to embodiment 1, wherein L is *—C(O)N(CH₃)—.

### Embodiment 7

A compound, or a salt, tautomer or stereoisomer thereof, according to embodiment 1, wherein L is *—CH₂N(CH₃)—.

### Embodiment 8

A compound, or a salt, tautomer or stereoisomer thereof, according to embodiment 1, wherein L is *—(CH₂)— or *—C(O)—.

### Embodiment 9

A compound, or a salt, tautomer or stereoisomer thereof, according to any one of embodiments 1 to 8, wherein Ring A is C₆₋₁₀aryl or C₅₋₁₀heteroaryl.

### Embodiment 10

A compound, or a salt, tautomer or stereoisomer thereof, according to any one of embodiments 1 to 8, wherein Ring A is selected from the group consisting of

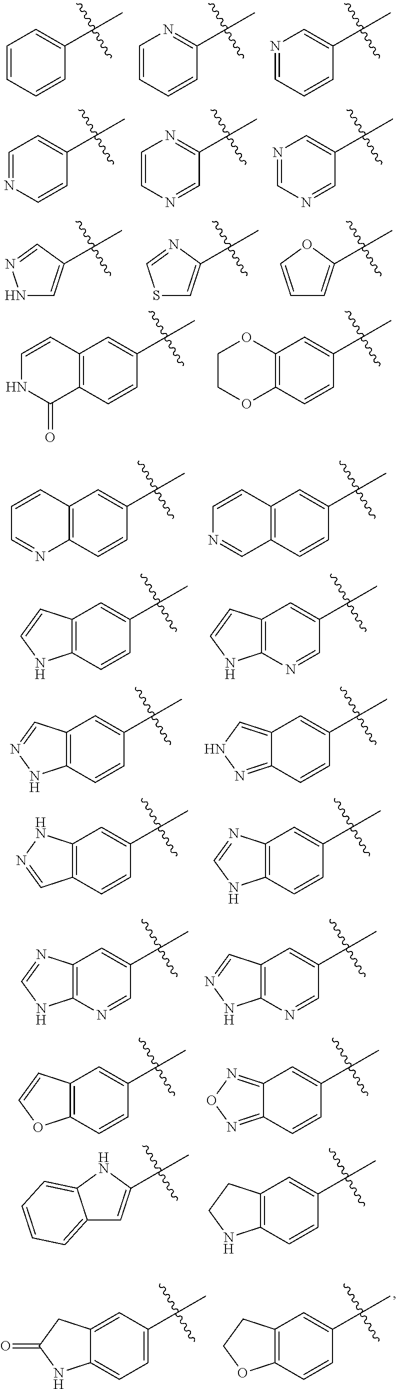

each of which is unsubstituted or substituted with 1 to 2 R₁ groups.

### Embodiment 11

A compound, or a salt, tautomer or stereoisomer thereof, according to any one of embodiments 1 to 8, wherein Ring A is selected from the group consisting of

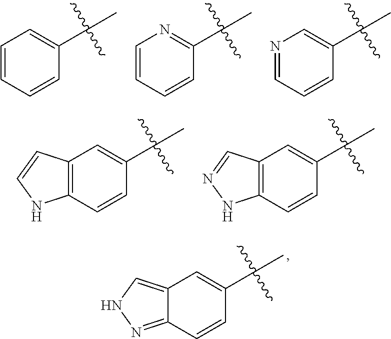 [SMILES: *c1ccccc1 ; *c1ccccn1 ; *c1cccnc1 ; *c1ccc2[nH]ccc2c1 ; *c1ccc2[nH]ncc2c1 ; *c1ccc2n[nH]cc2c1]

each of which is unsubstituted or substituted with 1 to 2 R₁ groups.

### Embodiment 12

A compound, or a salt, tautomer or stereoisomer thereof, according to any one of embodiments 1 to 8, wherein Ring A is phenyl or pyridinyl, each of which is unsubstituted or substituted with 1 to 2 R₁ groups.

### Embodiment 13

A compound, or a salt, tautomer or stereoisomer thereof, according to any one of embodiments 1 to 8, wherein Ring A is of the formula

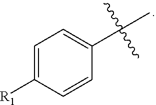

### Embodiment 14

A compound, or a salt, tautomer or stereoisomer thereof, according to any one of embodiments 1 to 8, wherein Ring A is of the formula

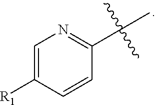

### Embodiment 15

A compound, or a salt, tautomer or stereoisomer thereof, according to any one of embodiments 1 to 8, wherein Ring A is of the formula

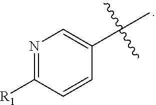

### Embodiment 16

A compound, or a salt, tautomer or stereoisomer thereof, according to any one of embodiments 1 to 7 and 9 to 15, wherein Ring C is selected from the group consisting of phenyl, C₅₋₁₀heteroaryl and C₅₋₁₀heterocycloalkyl.

### Embodiment 17

A compound, or a salt, tautomer or stereoisomer thereof, according to any one of embodiments 1 to 7 and 9 to 15, wherein Ring C is selected from the group consisting of

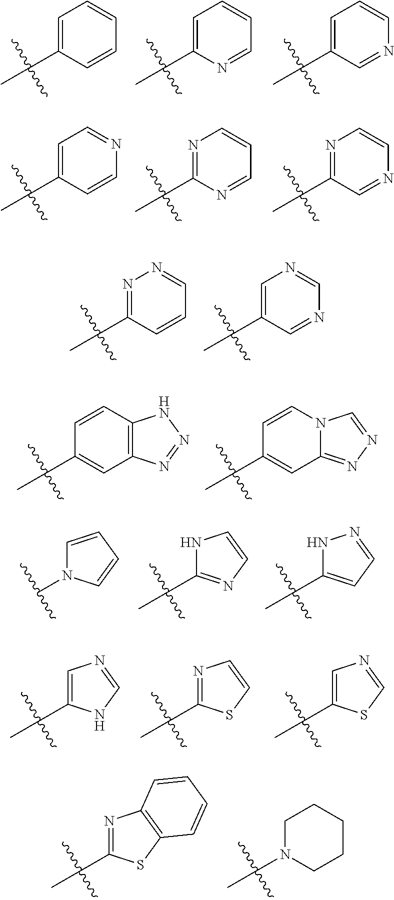

each of which is unsubstituted or substituted with 1 to 2 R₁₇ groups

### Embodiment 18

A compound, or a salt, tautomer or stereoisomer thereof, according to any one of embodiments 1 to 7 and 9 to 15, Ring C is selected from the group consisting of phenyl and pyridyl, each of which is unsubstituted or substituted with 1 to 2 R₁₇ groups.

### Embodiment 19

A compound, or a salt, tautomer or stereoisomer thereof, according to any one of embodiments 1 to 7 and 9 to 15, Ring C is of the formula:

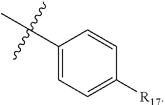

### Embodiment 20

A compound, or a salt, tautomer or stereoisomer thereof, according to any one of embodiments 1 to 7 and 9 to 15, Ring C is of the formula:

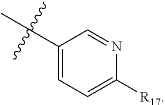

### Embodiment 21

A compound, or a salt, tautomer or stereoisomer thereof, according to any one of embodiments 1 to 7 and 9 to 15, Ring C is of the formula:

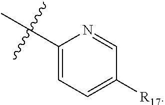

### Embodiment 22

A compound, or a salt, tautomer or stereoisomer thereof, according to any one of embodiments 1 to 3 and 8 to 15, wherein Ring C is C₆heterocycloalkyl or fused bicyclyl comprising a C₅₋₆heterocycloalkyl fused to a phenyl, wherein the C₆heterocycloalkyl and fused bicyclyl are each unsubstituted or substituted with 1 to 3 (R₁₇) group.

### Embodiment 23

A compound, or a salt, tautomer or stereoisomer thereof, according to any one of embodiments 1 to 3 and 8 to 15, wherein Ring C is selected from the group consisting of

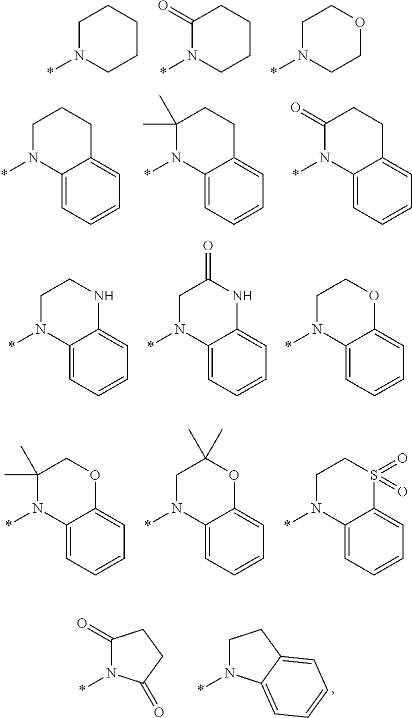

each of which is unsubstituted or substituted with 1 to 2 R₁₇ groups.

### Embodiment 24

A compound, or a salt, tautomer or stereoisomer thereof, according to any one of embodiments 1 to 3 and 8 to 15, wherein Ring C is selected from the group consisting of

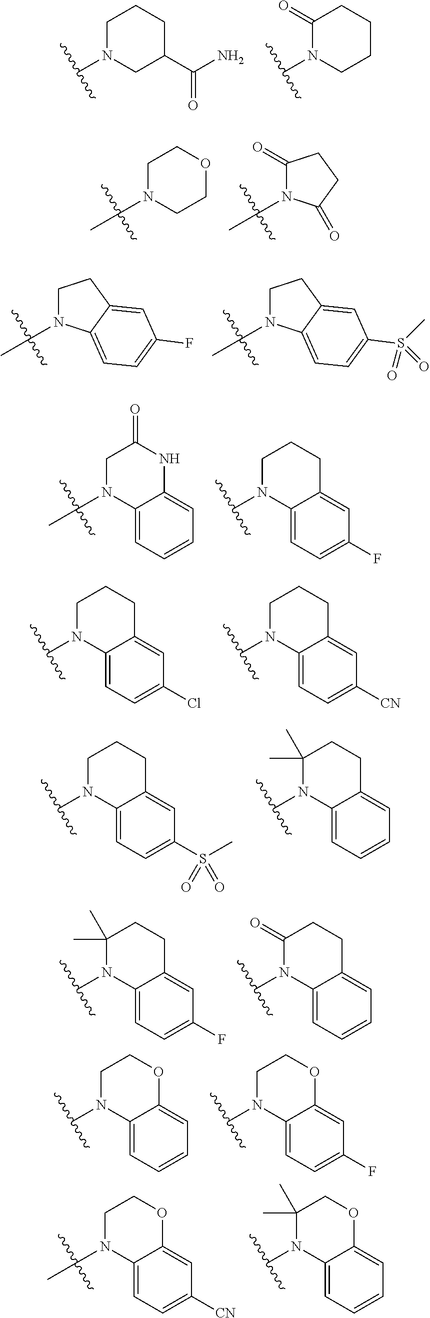

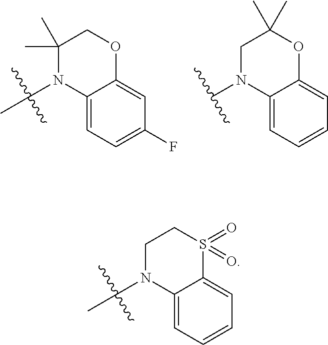 [SMILES: *N1c2ccc(F)cc2OCC1(C)C ; *N1CC(C)(C)Oc2ccccc21 ; *N1CCS(=O)(=O)c2ccccc21]

### Embodiment 25

A compound, or a salt, tautomer or stereoisomer thereof, according to any one of embodiments 1 to 24, wherein each R₁ is independently selected from the group consisting of fluoro, chloro, cyano, methyl, isopropyl, t-butyl, cyanopropyl, —CH(CH₃)(OCH₃), trifluoromethyl, difluoromethyl, —CF₂CH₃, —C(CH₃)₂CN, —CH₂NH₂, —CH₂N(CH₃)₂, —CH₂-morpholinyl, methoxy, proproxy, isoproproxy, difluoromethoxy, trifluoromethoxy, —OCH₂CF₃, cyanomethoxy, 2-aminoethoxy, —O(CH₂)₂N(CH₃)₂, —OC(CH₃)₂CH₂NH₂, —OC(CH₃)₂C(O)NH₂, and phenoxy.

### Embodiment 26

A compound, or a salt, tautomer or stereoisomer thereof, according to any one of embodiments 1 to 24, wherein each R₁ is independently selected from the group consisting of —C(O)CH₃, —C(O)OCH₃, —C(O)NH₂, —C(O)NHCH₃, —C(O)N(CH₃)₂, —C(O)N(CH₂CH₃)₂, —C(O)N(CH₃)(OCH₃), —C(O)NH((CH₂)₂N(CH₃)₂), —C(O)NH(cyclopropyl), and —C(O)NH(cyclohexyl).

### Embodiment 27

A compound, or a salt, tautomer or stereoisomer thereof, according to any one of embodiments 1 to 24, wherein each R₁ is independently selected from the group consisting of amino, methylamino, dimethylamino, —NH(OCH₃), —NH(CH₂C(NH₂)(CH₃)₂), —NH(C(CH₃)₂CH₂NH(C(O)OC(CH₃)₃)), —NH(C(CH₃)₂CH₂NH₂), —NH((CH₂)₂-morpholinyl), —NH(C(O)CH₃), —NH(C(O)CH₂NH₂), —NH(C(O)CH(NH₂)(CH₃)), —NH(C(O)CH(NH₂)CH(CH₃)₂), —NH(C(O)C(NH₂)(CH₃)₂), —NH(C(O)CH₂CH(NH₂)(CH₃)), —NH(C(O)CH₂CH(NH₂)CH(CH₃)₂), —NH(C(O)CH(NH₂)(cyclopropyl)), —NH(C(O)(1-aminomethylcyclopropyl)), —NH(C(O)CH(NH₂)-cyclobutyl), —NH(C(O)CH(NH₂)-cyclohexyl), —NH(C(O)-2-aminocyclopentyl), —NH(C(O)OCH₃), —NH(C(O)OCH₂CH₃), —NH(C(O)OCH(CH₃)₂), —NH(SO₂CH₃), and —NH(SO₂CH(CH₃)₂).

### Embodiment 28

A compound, or a salt, tautomer or stereoisomer thereof, according to any one of embodiments 1 to 24, wherein each R₁ is independently selected from the group consisting of —SO₂CH₃, —SO₂CH(CH₃)₂, —SO₂NH₂, and —SO₂N(CH₃)₂.

### Embodiment 29

A compound of the formula (I), or a salt, tautomer or stereoisomer thereof, according to any one of embodiments 1 to 24, wherein each R₁ is independently selected from the group consisting of

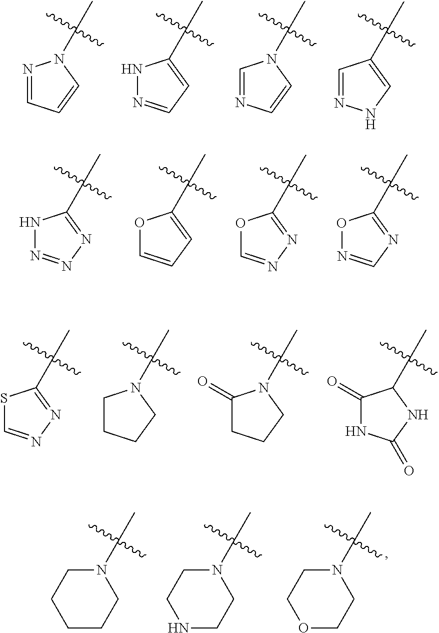

each of which is unsubstituted or substituted by 1 to 2 substituents independently selected from the group consisting of methyl, ethyl, —NH₂, —NH(CH₃), —C(O)CH₃, and benzyl.

### Embodiment 30

A compound, or a salt, tautomer or stereoisomer thereof, according to any one of embodiments 1 to 24, wherein each R₁ is independently selected from the group consisting of

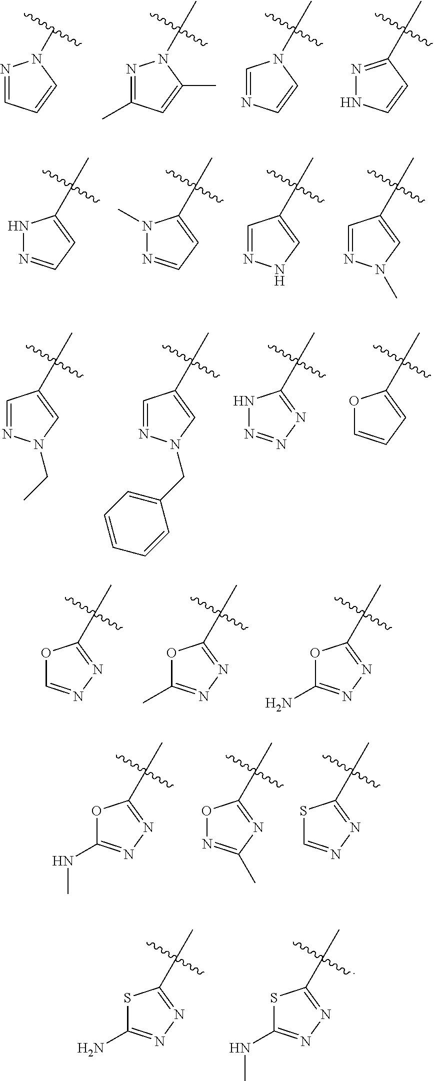

### Embodiment 31

A compound, or a salt, tautomer or stereoisomer thereof, according to any one of embodiments 1 to 24, wherein each R₁ is independently selected from the group consisting of

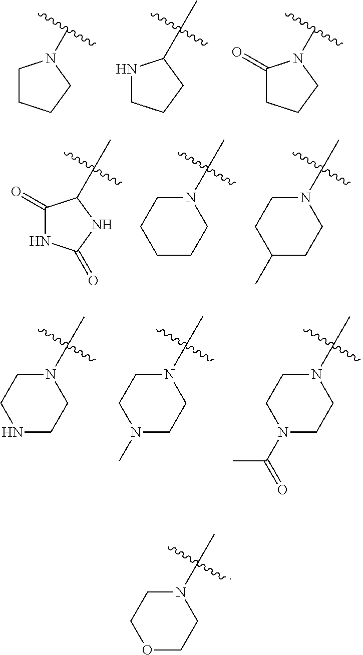

### Embodiment 32

A compound, or a salt, tautomer or stereoisomer thereof, according to any one of embodiments 1 to 24, wherein each R₁ is independently selected from the group consisting of trifluoromethyl, —C(O)NH₂, —C(O)NHCH₃, —C(O)NH(CH₂)₂N(CH₃)₂, and —NHC(O)CH(NH₂)-cycloalkyl.

### Embodiment 33

A compound, or a salt, tautomer or stereoisomer thereof, according to any one of embodiments 1 to 24, wherein R₁ is trifluoromethyl.

### Embodiment 34

A compound, or a salt, tautomer or stereoisomer thereof, according to any one of embodiments 1 to 24, wherein R₁ is —C(O)NH₂.

### Embodiment 35

A compound, or a salt, tautomer or stereoisomer thereof, according to any one of embodiments 1 to 24, wherein R₁ is —C(O)NHCH₃.

### Embodiment 36

A compound, or a salt, tautomer or stereoisomer thereof, according to any one of embodiments 1 to 35, wherein each R₁₇ is independently selected from the group consisting of fluoro, chloro, bromo, cyano, methyl, ethyl, t-butyl, trifluoromethyl, methoxymethyl, aminomethyl, methoxy, ethoxy, isopropoxy, difluoromethoxy, trifluoromethoxy, phenoxy, oxo, dimethylamino, methylsulfonyl, and aminocarbonyl.

### Embodiment 37

A compound, or a salt, tautomer or stereoisomer thereof, according to any one of embodiments 1 to 35, wherein each R₁₇ is independently selected from the group consisting of —C(O)CH₃, —C(O)NH₂, methylsulfonyl, —SO₂NH-thiazol-2-yl, and —SO₂NH₂.

### Embodiment 38

A compound, or a salt, tautomer or stereoisomer thereof, according to any one of embodiments 1 to 35, wherein each R₁₇ is independently selected from the group consisting of

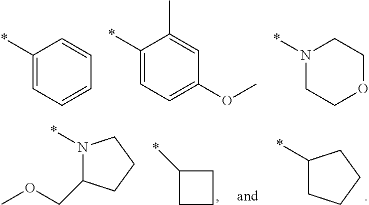

### Embodiment 39

A compound, or a salt, tautomer or stereoisomer thereof, according to any one of embodiments 1 to 35, wherein R₁₇ is cyano.

### Embodiment 40

A compound, or a salt, tautomer or stereoisomer thereof, according to any one of embodiments 1 to 35, wherein R₁₇ is chloro.

### Embodiment 41

A compound, or a salt, tautomer or stereoisomer thereof, according to any one of embodiments 1 to 35, wherein R₁₇ is fluoro.

### Embodiment 42

A compound, or a salt, tautomer or stereoisomer thereof, according to any one of embodiments 1 to 35, wherein R₁₇ is methylsulfonyl.

### Embodiment 43

A compound, or a salt, tautomer or stereoisomer thereof, according to any one of embodiments 1-15 and 21 to 23, wherein each of the R₁₇ is independently selected from the group consisting of fluoro, chloro, bromo, cyano, methyl, methylsulfonyl, and aminocarbonyl.

### Embodiment 44

A compound, or a salt, tautomer or stereoisomer thereof, according to of embodiment 1, wherein the compound is of Formula 1A:

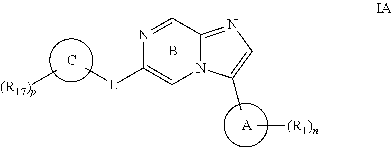

wherein
   n is 1;
   p is 1;
   L is *—C(O)N(R₂)— or *—CH₂N(R₂)—, wherein
      * represents the point of attachment of L to Ring B,
      R₂ is C₁₋₄alkyl or R₀—C₁₋₄alkylene, wherein R₀ is selected from the group consisting of C₁₋₄alkoxy, amino, C₁₋₄alkylamino, C₅₋₆heteroaryl and C₅₋₆heterocycloalkyl, wherein said C₅₋₆heteroaryl and C₅₋₆heterocycloalkyl are each independently unsubstituted or substituted by 1-2 substituents independently selected from the group consisting of C₁₋₄alkyl, halo, amino, and oxo;
Ring A is phenyl or pyridyl;
Ring C is phenyl or pyridyl;
each R₁ is trifluoromethyl, *—C(O)NH₂, or *—C(O)NHCH₃; and
each R₁₇ is chloro, fluoro, or cyano.

### Embodiment 45

A compound, or a salt, tautomer or stereoisomer thereof, according to any one of embodiments 44, wherein L is *—C(O)N(CH₃)—.

### Embodiment 46

A compound, or a salt, tautomer or stereoisomer thereof, according to any one of embodiments 44, wherein L is *—CH₂N(CH₃)—.

### Embodiment 47

A compound, or a salt, tautomer or stereoisomer thereof, according to any one of embodiments 44 to 46, Ring A is of the formula:

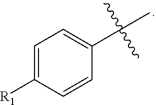

### Embodiment 48

A compound, or a salt, tautomer or stereoisomer thereof, according to any one of embodiments 44 to 46, Ring A is of the formula:

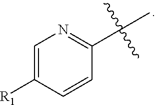

### Embodiment 49

A compound, or a salt, tautomer or stereoisomer thereof, according to any one of embodiments 44 to 46, Ring A is of the formula:

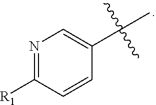

### Embodiment 50

A compound, or a salt, tautomer or stereoisomer thereof, according to any one of embodiments 44 to 49, Ring C is of the formula:

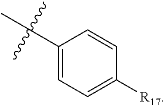

### Embodiment 51

A compound, or a salt, tautomer or stereoisomer thereof, according to any one of embodiments 44 to 49, Ring C is of the formula:

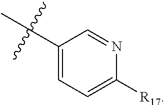

### Embodiment 52

A compound, or a salt, tautomer or stereoisomer thereof, according to any one of embodiments 44 to 49, Ring C is of the formula:

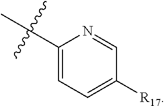

### Embodiment 53

A compound, or a salt, tautomer or stereoisomer thereof, according to embodiment 1, wherein the compound is selected from the group consisting of: N-(4-cyanophenyl)-3-(6-(3-(dimethylamino)propoxy)pyridin-3-yl)-N-methylimidazo[1,2-a]pyrazine-6-carboxamide; 4-(((3-(4-(1H-pyrazol-5-yl)phenyl)imidazo[1,2-a]pyrazin-6-yl)methyl)(methyl)amino)benzonitrile; N-((3-(4-(1H-pyrazol-5-yl)phenyl)imidazo[1,2-a]pyrazin-6-yl)methyl)-4-fluoro-N-methylaniline; 4-(((3-(4-chlorophenyl)imidazo[1,2-a]pyrazin-6-yl)methyl)(methyl)amino)benzonitrile; N-((3-(4-(1H-pyrazol-5-yl)phenyl)imidazo[1,2-a]pyrazin-6-yl)methyl)-4-chloro-N-methylaniline; N-(5-chloro-3-fluoropyridin-2-yl)-N-methyl-3-(4-(trifluoromethyl)phenyl)imidazo[1,2-a]pyrazine-6-carboxamide; 5-methanesulfonyl-1-({3-[4-(trifluoromethyl)phenyl]imidazo[1,2-a]pyrazin-6-yl}carbonyl)-2,3-dihydro-1H-indole; 4-fluoro-N-methyl-N-((3-(1-methyl-1H-indazol-5-yl)imidazo[1,2-a]pyrazin-6-yl)methyl)aniline; N-((3-(4-(1H-pyrazol-1-yl)phenyl)imidazo[1,2-a]pyrazin-6-yl)methyl)-4-chloro-N-methylaniline; N-((3-(4-(1H-pyrazol-1-yl)phenyl)imidazo[1,2-a]pyrazin-6-yl)methyl)-4-chloro-N-methylaniline; 3-(4-carbamoylphenyl)-N-(4-fluorophenyl)-N-methylimidazo[1,2-a]pyrazine-6-carboxamide; N-methyl-N-(1-methyl-1H-benzo[d][1,2,3]triazol-5-yl)-3-(4-(trifluoromethyl)phenyl)imidazo[1,2-a]pyrazine-6-carboxamide; N-(4-chlorophenyl)-N-methyl-3-[4-(1H-pyrazol-5-yl)phenyl]imidazo[1,2-a]pyrazine-6-carboxamide; N-(1,3-benzothiazol-2-yl)-N-methyl-3-[4-(trifluoromethyl)phenyl]imidazo[1,2-a]pyrazine-6-carboxamide; [4-(5-amino-1,3,4-oxadiazol-2-yl)phenyl]-N-(4-chlorophenyl)-N-methylimidazo[1,2-a]pyrazine-6-carboxamide; N-(3-methoxypyridin-2-yl)-N-methyl-3-[4-(trifluoromethyl)phenyl]imidazo[1,2-a]pyrazine-6-carboxamide; 3-(4-chlorophenyl)-N-(4-methanesulfonylphenyl)-N-methylimidazo[1,2-a]pyrazine-6-carboxamide; N-{[3-(4-chlorophenyl)imidazo[1,2-a]pyrazin-6-yl]methyl}-4-fluoro-N-methylaniline; N-(4-cyanophenyl)-N-methyl-3-(5-methylpyridin-2-yl)imidazo[1,2-a]pyrazine-6-carboxamide; N-(4-cyanophenyl)-3-(2,4-dichlorophenyl)-N-methylimidazo[1,2-a]pyrazine-6-carboxamide; N-({3-[4-(difluoromethoxy)phenyl]imidazo[1,2-a]pyrazin-6-yl}methyl)-4-fluoro-N-methylaniline; 3-(4-carbamoylphenyl)-N-(3,4-difluorophenyl)-N-methylimidazo[1,2-a]pyrazine-6-carboxamide; N-(2,4-difluorophenyl)-N-methyl-3-[4-(trifluoromethyl)phenyl]imidazo[1,2-a]pyrazine-6-carboxamide; N-methyl-4-(morpholin-4-yl)-N-{3-[4-(trifluoromethyl)phenyl]imidazo[1,2-a]pyrazin-6-yl}benzamide; 4-[({3-[4-(5-amino-1,3,4-thiadiazol-2-yl)phenyl]imidazo[1,2-a]pyrazin-6-yl}methyl)(methyl)amino]benzonitrile; 3-(4-chloro-2-methylphenyl)-N-(4-cyanophenyl)-N-methylimidazo[1,2-a]pyrazine-6-carboxamide; 3-(2-chloropyridin-4-yl)-N-(4-cyanophenyl)-N-methylimidazo[1,2-a]pyrazine-6-carboxamide; ({3-[4-(5-amino-1,3,4-thiadiazol-2-yl)phenyl]imidazo[1,2-a]pyrazin-6-yl}methyl)-6-chloro-N-methylpyridin-3-amine; N-(4-cyanophenyl)-3-(3,5-difluoropyridin-2-yl)-N-methylimidazo[1,2-a]pyrazine-6-carboxamide; N-(4-cyanophenyl)-3-(4-methoxy-3-methylphenyl)-N-methylimidazo[1,2-a]pyrazine-6-carboxamide; 3-[2-chloro-4-(trifluoromethyl)phenyl]-N-(4-cyanophenyl)-N-methylimidazo[1,2-a]pyrazine-6-carboxamide; N-(3-chloro-4-fluorophenyl)-N-methyl-3-[4-(trifluoromethyl)phenyl]imidazo[1,2-a]pyrazine-6-carboxamide; 3-acetyl-N-(6-chloropyridin-3-yl)-N-methylimidazo[1,2-a]pyrazine-6-carboxamide; 4-chloro-N-methyl-N-{[3-(1-methyl-1H-indazol-5-yl)imidazo[1,2-a]pyrazin-6-yl]methyl}aniline; N-(3-chlorophenyl)-N-methyl-3-[4-(trifluoromethyl)phenyl]imidazo[1,2-a]pyrazine-6-carboxamide; 4-(6-{[(4-fluorophenyl)(methyl)amino]methyl}imidazo[1,2-a]pyrazin-3-yl)-N,N-dimethylaniline; 4-(6-{[(4-chlorophenyl)(methyl)amino]methyl}imidazo[1,2-a]pyrazin-3-yl)-N,N-dimethylaniline; N-(4-cyanophenyl)-N-methyl-3-[4-(methylcarbamoyl)phenyl]imidazo[1,2-a]pyrazine-6-carboxamide; N-(5-ethylpyridin-2-yl)-N-methyl-3-[4-(trifluoromethyl)phenyl]imidazo[1,2-a]pyrazine-6-carboxamide; N-methyl-N-[2-(trifluoromethoxy)phenyl]-3-[4-(trifluoromethyl)phenyl]imidazo[1,2-a]pyrazine-6-carboxamide; N-(2,4-dichlorophenyl)-3-(4-methoxyphenyl)-N-methylimidazo[1,2-a]pyrazine-6-carboxamide; N-(3-chloro-4-methoxyphenyl)-N-(4-cyanophenyl)-N-methylimidazo[1,2-a]pyrazine-6-carboxamide; N-methyl-N-(4-phenoxyphenyl)-3-[4-(trifluoromethyl)phenyl]imidazo[1,2-a]pyrazine-6-carboxamide; 4-chloro-N-{[3-(4-chlorophenyl)imidazo[1,2-a]pyrazin-6-yl]methyl}-N-methylaniline; N,5-dimethyl-N-{[3-(1-methyl-1H-indazol-5-yl)imidazo[1,2-a]pyrazin-6-yl]methyl}pyridin-2-amine; N-(4-cyanophenyl)-N-methyl-3-[3-(1H-pyrazol-4-yl)phenyl]imidazo[1,2-a]pyrazine-6-carboxamide; N,5-dimethyl-N-({3-[4-(1H-pyrazol-5-yl)phenyl]imidazo[1,2-a]pyrazin-6-yl}methyl)pyridin-2-amine; N-(4-cyanophenyl)-3-(3-methoxyphenyl)-N-methylimidazo[1,2-a]pyrazine-6-carboxamide; N-(2,4-dichlorophenyl)-N-methyl-3-[4-(trifluoromethyl)phenyl]imidazo[1,2-a]pyrazine-6-carboxamide; N-[4-(difluoromethoxy)phenyl]-N-methyl-3-[4-(trifluoromethyl)phenyl]imidazo[1,2-a]pyrazine-6-carboxamide; N-methyl-N-[4-(1-methyl-1H-pyrazol-4-yl)phenyl]-3-[4-(trifluoromethyl)phenyl]imidazo[1,2-a]pyrazine-6-carboxamide; N-(3-bromo-4-fluorophenyl)-N-methyl-3-[4-(trifluoromethyl)phenyl]imidazo[1,2-a]pyrazine-6-carboxamide; N-(3,4-difluorophenyl)-N-methyl-3-(1-methyl-1H-indazol-5-yl)imidazo[1,2-a]pyrazine-6-carboxamide; N-(2-fluoropyridin-3-yl)-N-methyl-3-[4-(trifluoromethyl)phenyl]imidazo[1,2-a]pyrazine-6-carboxamide; N-(3,4-dichlorophenyl)-N-methyl-3-[4-(trifluoromethyl)phenyl]imidazo[1,2-a]pyrazine-6-carboxamide; N-(4-chloro-3-methylphenyl)-N-methyl-3-[4-(trifluoromethyl)phenyl]imidazo[1,2-a]pyrazine-6-carboxamide; N-(4-cyanophenyl)-3-[4-(difluoromethyl)phenyl]-N-methylimidazo[1,2-a]pyrazine-6-carboxamide; N-(6-bromopyridin-3-yl)-N-methyl-3-[4-(trifluoromethyl)phenyl]imidazo[1,2-a]pyrazine-6-carboxamide; N-(4-chloro-2-methoxyphenyl)-N-methyl-3-[4-(trifluoromethyl)phenyl]imidazo[1,2-a]pyrazine-6-carboxamide; 3-[4-chloro-3-(trifluoromethyl)phenyl]-N-(4-cyanophenyl)-N-methylimidazo[1,2-a]pyrazine-6-carboxamide; N-(2,4-dichlorophenyl)-N-methyl-3-(4-methylphenyl)imidazo[1,2-a]pyrazine-6-carboxamide; 4-[methyl({[3-(1-methyl-1H-indazol-5-yl)imidazo[1,2-a]pyrazin-6-yl]methyl})amino]benzonitrile; N-(5-fluoropyridin-2-yl)-N-methyl-3-[4-(trifluoromethyl)phenyl]imidazo[1,2-a]pyrazine-6-carboxamide; 4-chloro-N-({3-[4-(difluoromethoxy)phenyl]imidazo[1,2-a]pyrazin-6-yl}methyl)-N-methylaniline; N-(4-cyanophenyl)-3-(2-fluoro-4-methoxyphenyl)-N-methylimidazo[1,2-a]pyrazine-6-carboxamide; N-(5-fluoropyridin-3-yl)-N-methyl-3-[4-(trifluoromethyl)phenyl]imidazo[1,2-a]pyrazine-6-carboxamide; N-(4-cyanophenyl)-3-(4-methoxyphenyl)-N-methylimidazo[1,2-a]pyrazine-6-carboxamide; N-methyl-N-[4-(2-methyl-2H-1,2,3,4-tetrazol-5-yl)phenyl]-3-[4-(trifluoromethyl)phenyl]imidazo[1,2-a]pyrazine-6-carboxamide; N-(4-fluorophenyl)-3-(4-methoxyphenyl)-N-methylimidazo[1,2-a]pyrazine-6-carboxamide; 3-(4-chlorophenyl)-N-(2,4-dichlorophenyl)-N-methylimidazo[1,2-a]pyrazine-6-carboxamide; 3-(4-chloro-3-fluorophenyl)-N-(4-cyanophenyl)-N-methylimidazo[1,2-a]pyrazine-6-carboxamide; N-(4-cyanophenyl)-N-[2-(dimethylamino)ethyl]-3-[4-(trifluoromethyl)phenyl]imidazo[1,2-a]pyrazine-6-carboxamide; N-(2-chlorophenyl)-N-methyl-3-[4-(trifluoromethyl)phenyl]imidazo[1,2-a]pyrazine-6-carboxamide; 3-[4-(5-amino-1,3,4-thiadiazol-2-yl)phenyl]-N-(4-cyanophenyl)-N-methylimidazo[1,2-a]pyrazine-6-carboxamide; N-(4-cyanophenyl)-3-[4-(1,1-difluoroethyl)phenyl]-N-methylimidazo[1,2-a]pyrazine-6-carboxamide; N-(4-cyanophenyl)-N-methyl-3-[4-(trifluoromethyl)phenyl]imidazo[1,2-a]pyrazine-6-carboxamide; N-(4-cyanophenyl)-3-[4-(dimethylamino)phenyl]-N-methylimidazo[1,2-a]pyrazine-6-carboxamide; N-(6-chloropyridin-3-yl)-N-methyl-3-[4-(trifluoromethyl)phenyl]imidazo[1,2-a]pyrazine-6-carboxamide; N-(6-fluoropyridin-3-yl)-N-methyl-3-[4-(trifluoromethyl)phenyl]imidazo[1,2-a]pyrazine-6-carboxamide; N-(6-chloropyridin-3-yl)-N-methyl-3-(4-methylphenyl)imidazo[1,2-a]pyrazine-6-carboxamide; N-(4-chlorophenyl)-N-(6-chloropyridin-3-yl)-N-methylimidazo[1,2-a]pyrazine-6-carboxamide; N-(4-cyanophenyl)-N-methyl-3-[6-(pyrrolidin-1-yl)pyridin-3-yl]imidazo[1,2-a]pyrazine-6-carboxamide; N-(5-cyanopyridin-2-yl)-N-methyl-3-(1-methyl-1H-indazol-5-yl)imidazo[1,2-a]pyrazine-6-carboxamide; N-(6-chloropyridin-3-yl)-N-methyl-3-(1-methyl-1H-indol-5-yl)imidazo[1,2-a]pyrazine-6-carboxamide; N-(5-cyanopyridin-2-yl)-3-[4-(difluoromethoxy)phenyl]-N-methylimidazo[1,2-a]pyrazine-6-carboxamide; N-(6-fluoropyridin-2-yl)-N-methyl-3-[4-(trifluoromethyl)phenyl]imidazo[1,2-a]pyrazine-6-carboxamide; N-(4-cyanophenyl)-N-methyl-3-[4-(1H-pyrazol-1-yl)phenyl]imidazo[1,2-a]pyrazine-6-carboxamide; N-(6-methoxypyridin-3-yl)-N-methyl-3-[4-(trifluoromethyl)phenyl]imidazo[1,2-a]pyrazine-6-carboxamide; N-(5-cyanopyridin-2-yl)-N-methyl-3-[4-(trifluoromethyl)phenyl]imidazo[1,2-a]pyrazine-6-carboxamide; N-(4-cyanophenyl)-3-(6-methoxypyridin-3-yl)-N-methylimidazo[1,2-a]pyrazine-6-carboxamide; N-(5-chloropyrazin-2-yl)-N-methyl-3-[4-(trifluoromethyl)phenyl]imidazo[1,2-a]pyrazine-6-carboxamide; N-(4-cyanophenyl)-N-(propan-2-yl)-3-[4-(trifluoromethyl)phenyl]imidazo[1,2-a]pyrazine-6-carboxamide; N-(5-chloropyrimidin-2-yl)-N-methyl-3-[4-(trifluoromethyl)phenyl]imidazo[1,2-a]pyrazine-6-carboxamide; N-(6-chloropyridazin-3-yl)-N-methyl-3-[4-(trifluoromethyl)phenyl]imidazo[1,2-a]pyrazine-6-carboxamide; N-(5-cyanopyridin-2-yl)-3-[4-(difluoromethyl)phenyl]-N-methylimidazo[1,2-a]pyrazine-6-carboxamide; N-(6-bromopyridazin-3-yl)-N-methyl-3-[4-(trifluoromethyl)phenyl]imidazo[1,2-a]pyrazine-6-carboxamide; N-(5-bromopyrazin-2-yl)-N-methyl-3-[4-(trifluoromethyl)phenyl]imidazo[1,2-a]pyrazine-6-carboxamide; 3-(4-carbamoylphenyl)-N-(5-cyanopyridin-2-yl)-N-methylimidazo[1,2-a]pyrazine-6-carboxamide; N-methyl-N-(pyridin-2-yl)-3-[4-(trifluoromethyl)phenyl]imidazo[1,2-a]pyrazine-6-carboxamide; N-(6-chloropyridin-3-yl)-3-(4-methoxyphenyl)-N-methylimidazo[1,2-a]pyrazine-6-carboxamide; N-(4-fluorophenyl)-N-methyl-3-[4-(trifluoromethyl)phenyl]imidazo[1,2-a]pyrazine-6-carboxamide; N-(4-chlorophenyl)-3-[4-(difluoromethoxy)phenyl]-N-methylimidazo[1,2-a]pyrazine-6-carboxamide; 3-(4-chlorophenyl)-N-(4-fluorophenyl)-N-methylimidazo[1,2-a]pyrazine-6-carboxamide; 3-(4-carbamoylphenyl)-N-(4-cyanophenyl)-N-methylimidazo[1,2-a]pyrazine-6-carboxamide; N-(3,4-difluorophenyl)-N-methyl-3-[4-(trifluoromethyl)phenyl]imidazo[1,2-a]pyrazine-6-carboxamide; N-(4-fluorophenyl)-N-methyl-3-(1-methyl-1H-indazol-5-yl)imidazo[1,2-a]pyrazine-6-carboxamide; N-(4-fluorophenyl)-N-methyl-3-(1-methyl-1H-indazol-5-yl)imidazo[1,2-a]pyrazine-6-carboxamide; N-(4-fluorophenyl)-N-methyl-3-(4-methylphenyl)imidazo[1,2-a]pyrazine-6-carboxamide; N,3-bis(4-chlorophenyl)-N-methylimidazo[1,2-a]pyrazine-6-carboxamide; N-(2-methoxyphenyl)-N-methyl-3-[4-(trifluoromethyl)phenyl]imidazo[1,2-a]pyrazine-6-carboxamide; 6-fluoro-1-({3-[4-(trifluoromethyl)phenyl]imidazo[1,2-a]pyrazin-6-yl}carbonyl)-1,2,3,4-tetrahydroquinoline; 3-(4-chlorophenyl)-N-(3,4-difluorophenyl)-N-methylimidazo[1,2-a]pyrazine-6-carboxamide; N-(4-cyanophenyl)-N-methyl-3-(4-methylphenyl)imidazo[1,2-a]pyrazine-6-carboxamide; N-(4-cyanophenyl)-N-ethyl-3-[4-(trifluoromethyl)phenyl]imidazo[1,2-a]pyrazine-6-carboxamide; N-(4-cyanophenyl)-N-methyl-3-[4-(propan-2-yl)phenyl]imidazo[1,2-a]pyrazine-6-carboxamide; N-(4-cyanophenyl)-N-methyl-3-[4-(trifluoromethoxy)phenyl]imidazo[1,2-a]pyrazine-6-carboxamide; N-(4-cyanophenyl)-N-methyl-3-[4-(methylamino)phenyl]imidazo[1,2-a]pyrazine-6-carboxamide; 3-(4-tert-butylphenyl)-N-(4-cyanophenyl)-N-methylimidazo[1,2-a]pyrazine-6-carboxamide; N-(2-fluorophenyl)-N-methyl-3-[4-(trifluoromethyl)phenyl]imidazo[1,2-a]pyrazine-6-carboxamide; N-[4-(1-cyano-1-methylethyl)phenyl]-N-(4-cyanophenyl)-N-methylimidazo[1,2-a]pyrazine-6-carboxamide; 3-[4-(dimethylamino)phenyl]-N-(4-fluorophenyl)-N-methylimidazo[1,2-a]pyrazine-6-carboxamide; N-(4-chlorophenyl)-3-[4-(dimethylamino)phenyl]-N-methylimidazo[1,2-a]pyrazine-6-carboxamide; N-(4-cyanophenyl)-N-methyl-3-(1-methyl-1H-indol-5-yl)imidazo[1,2-a]pyrazine-6-carboxamide; 3-(4-aminophenyl)-N-(4-cyanophenyl)-N-methylimidazo[1,2-a]pyrazine-6-carboxamide; N-(4-cyanophenyl)-3-[4-(furan-2-yl)phenyl]-N-methylimidazo[1,2-a]pyrazine-6-carboxamide; N-(4-cyano-3-fluorophenyl)-N-methyl-3-[4-(trifluoromethyl)phenyl]imidazo[1,2-a]pyrazine-6-carboxamide; N-(3,4-difluorophenyl)-N-methyl-3-(1-methyl-1H-indol-5-yl)imidazo[1,2-a]pyrazine-6-carboxamide; N-(4-methanesulfonylphenyl)-3-[4-(trifluoromethyl)phenyl]imidazo[1,2-a]pyrazine-6-carboxamide; N-(4-cyanophenyl)-3-(4-methanesulfonylphenyl)-N-methylimidazo[1,2-a]pyrazine-6-carboxamide; N-(4-cyanophenyl)-3-[4-(1-methoxyethyl)phenyl]-N-methylimidazo[1,2-a]pyrazine-6-carboxamide; N-(4-methanesulfonylphenyl)-N-methyl-3-[4-(1H-pyrazol-5-yl)phenyl]imidazo[1,2-a]pyrazine-6-carboxamide; 3-(4-chlorophenyl)-N-(4-cyanophenyl)-N-methylimidazo[1,2-a]pyrazine-6-carboxamide; N-(4-cyanophenyl)-N-[2-(oxan-4-yl)ethyl]-3-[4-(trifluoromethyl)phenyl]imidazo[1,2-a]pyrazine-6-carboxamide; 3-(4-acetylphenyl)-N-(4-cyanophenyl)-N-methylimidazo[1,2-a]pyrazine-6-carboxamide; N-(3,4-difluorophenyl)-N-methyl-3-(4-methylphenyl)imidazo[1,2-a]pyrazine-6-carboxamide; N-(4-fluorophenyl)-N-methyl-3-(1-methyl-1H-indol-5-yl)imidazo[1,2-a]pyrazine-6-carboxamide; N-(3-fluorophenyl)-N-methyl-3-[4-(trifluoromethyl)phenyl]imidazo[1,2-a]pyrazine-6-carboxamide; N-(4-cyanophenyl)-N-methyl-3-[4-(2,2,2-trifluoroethoxy)phenyl]imidazo[1,2-a]pyrazine-6-carboxamide; N-(4-methoxyphenyl)-N-methyl-3-[4-(trifluoromethyl)phenyl]imidazo[1,2-a]pyrazine-6-carboxamide; N-(4-cyanophenyl)-3-[2-fluoro-4-(trifluoromethyl)phenyl]-N-methylimidazo[1,2-a]pyrazine-6-carboxamide; N-(4-chlorophenyl)-N-methyl-3-(1-methyl-1H-indol-5-yl)imidazo[1,2-a]pyrazine-6-carboxamide; 3-[4-(difluoromethoxy)phenyl]-N-(3,4-difluorophenyl)-N-methylimidazo[1,2-a]pyrazine-6-carboxamide; 3-(1H-1,3-benzodiazol-5-yl)-N-(4-cyanophenyl)-N-methylimidazo[1,2-a]pyrazine-6-carboxamide; N-(3,4-difluorophenyl)-N-methyl-3-[4-(1H-pyrazol-1-yl)phenyl]imidazo[1,2-a]pyrazine-6-carboxamide; N-(4-cyanophenyl)-3-(1H-indazol-5-yl)-N-methylimidazo[1,2-a]pyrazine-6-carboxamide; N-(6-cyanopyridin-3-yl)-N-methyl-3-[4-(trifluoromethyl)phenyl]imidazo[1,2-a]pyrazine-6-carboxamide; N-(6-cyanopyridin-3-yl)-N-methyl-3-[4-(trifluoromethyl)phenyl]imidazo[1,2-a]pyrazine-6-carboxamide; N-(4-cyanophenyl)-N-methyl-3-(2-methyl-2H-indazol-5-yl)imidazo[1,2-a]pyrazine-6-carboxamide; N-(4-cyanophenyl)-N-methyl-3-(2-methyl-2H-indazol-5-yl)imidazo[1,2-a]pyrazine-6-carboxamide; N-(4-cyanophenyl)-N-methyl-3-[2-methyl-4-(trifluoromethyl)phenyl]imidazo[1,2-a]pyrazine-6-carboxamide; N-(4-fluorophenyl)-N-methyl-3-[4-(1H-pyrazol-1-yl)phenyl]imidazo[1,2-a]pyrazine-6-carboxamide; N-(4-cyanophenyl)-N-methyl-3-[4-(propane-2-sulfonyl)phenyl]imidazo[1,2-a]pyrazine-6-carboxamide; N-(4-cyanophenyl)-3-(4-fluorophenyl)-N-methylimidazo[1,2-a]pyrazine-6-carboxamide; N-(4-cyanophenyl)-3-(1H-indazol-6-yl)-N-methylimidazo[1,2-a]pyrazine-6-carboxamide; 3-(4-chloro-3-cyanophenyl)-N-(4-cyanophenyl)-N-methylimidazo[1,2-a]pyrazine-6-carboxamide; N-(4-cyanophenyl)-N-methyl-3-[2-(propan-2-yl)-1,3-thiazol-4-yl]imidazo[1,2-a]pyrazine-6-carboxamide; N-(4-cyanophenyl)-N-methyl-3-(quinolin-6-yl)imidazo[1,2-a]pyrazine-6-carboxamide; N-(4-cyanophenyl)-3-[4-(cyclopropylcarbamoyl)phenyl]-N-methylimidazo[1,2-a]pyrazine-6-carboxamide; N-(3,4-difluorophenyl)-3-[4-(dimethylamino)phenyl]-N-methylimidazo[1,2-a]pyrazine-6-carboxamide; N-(4-cyanophenyl)-3-[4-(3,5-dimethyl-1H-pyrazol-1-yl)phenyl]-N-methylimidazo[1,2-a]pyrazine-6-carboxamide; 3-(4-carbamoylphenyl)-N-(6-chloropyridin-3-yl)-N-methylimidazo[1,2-a]pyrazine-6-carboxamide; N-(4-cyanophenyl)-N-methyl-3-[4-(propan-2-yloxy)phenyl]imidazo[1,2-a]pyrazine-6-carboxamide; N-(4-cyanophenyl)-N-methyl-3-[4-(5-methyl-1,3,4-oxadiazol-2-yl)phenyl]imidazo[1,2-a]pyrazine-6-carboxamide; 3-[4-(dimethylamino)phenyl]-N-(4-methanesulfonylphenyl)-N-methylimidazo[1,2-a]pyrazine-6-carboxamide; N-(4-cyanophenyl)-3-[4-(2,5-dioxoimidazolidin-4-yl)phenyl]-N-methylimidazo[1,2-a]pyrazine-6-carboxamide; methyl 4-{6-[(4-cyanophenyl)(methyl)carbamoyl]imidazo[1,2-a]pyrazin-3-yl}benzoate; N-(4-cyanophenyl)-3-(isoquinolin-6-yl)-N-methylimidazo[1,2-a]pyrazine-6-carboxamide; N-(4-cyanophenyl)-N-methyl-3-(1-oxo-1,2-dihydroisoquinolin-6-yl)imidazo[1,2-a]pyrazine-6-carboxamide; N-(4-cyanophenyl)-3-[3-fluoro-4-(trifluoromethyl)phenyl]-N-methylimidazo[1,2-a]pyrazine-6-carboxamide; N-(4-cyanophenyl)-N-methyl-3-(6-propoxypyridin-3-yl)imidazo[1,2-a]pyrazine-6-carboxamide; N-methyl-N-[2-(trifluoromethyl)phenyl]-3-[4-(trifluoromethyl)phenyl]imidazo[1,2-a]pyrazine-6-carboxamide; 3-(4-carbamoylphenyl)-N-(4-methanesulfonylphenyl)-N-methylimidazo[1,2-a]pyrazine-6-carboxamide; N-methyl-N-[4-(trifluoromethoxy)phenyl]-3-[4-(trifluoromethyl)phenyl]imidazo[1,2-a]pyrazine-6-carboxamide; N-methyl-N-(1-methyl-1H-imidazol-2-yl)-3-[4-(trifluoromethyl)phenyl]imidazo[1,2-a]pyrazine-6-carboxamide; N-(4-cyanophenyl)-N-methyl-3-[3-(trifluoromethyl)phenyl]imidazo[1,2-a]pyrazine-6-carboxamide; 5-fluoro-1-({3-[4-(trifluoromethyl)phenyl]imidazo[1,2-a]pyrazin-6-yl}carbonyl)-2,3-dihydro-1H-indole; N-(4-cyanophenyl)-N-methyl-3-(4-phenoxyphenyl)imidazo[1,2-a]pyrazine-6-carboxamide; 3-(1-benzyl-1H-pyrazol-4-yl)-N-(4-cyanophenyl)-N-methylimidazo[1,2-a]pyrazine-6-carboxamide; 3-[4-chloro-2-(trifluoromethyl)phenyl]-N-(4-cyanophenyl)-N-methylimidazo[1,2-a]pyrazine-6-carboxamide; N-(4-cyanophenyl)-N-methyl-3-[4-(1H-1,2,3,4-tetrazol-5-yl)phenyl]imidazo[1,2-a]pyrazine-6-carboxamide; N-(4-cyanophenyl)-N-methyl-3-[4-(pyrrolidin-1-yl)phenyl]imidazo[1,2-a]pyrazine-6-carboxamide; N-(5-cyanopyridin-2-yl)-N-methyl-3-[4-(1H-pyrazol-5-yl)phenyl]imidazo[1,2-a]pyrazine-6-carboxamide; N-(3-bromophenyl)-N-methyl-3-[4-(trifluoromethyl)phenyl]imidazo[1,2-a]pyrazine-6-carboxamide; N-(4-cyanophenyl)-N-methyl-3-(5-sulfamoylpyridin-3-yl)imidazo[1,2-a]pyrazine-6-carboxamide; N-(4-cyanophenyl)-3-{4-[methoxy(methyl)carbamoyl]phenyl}-N-methylimidazo[1,2-a]pyrazine-6-carboxamide; 3-[4-(aminomethyl)phenyl]-N-(4-cyanophenyl)-N-methylimidazo[1,2-a]pyrazine-6-carboxamide; N-(4-cyanophenyl)-N-methyl-3-[6-(trifluoromethyl)pyridin-3-yl]imidazo[1,2-a]pyrazine-6-carboxamide; N-(4-cyanophenyl)-3-[4-(diethylcarbamoyl)phenyl]-N-methylimidazo[1,2-a]pyrazine-6-carboxamide; N-(4-cyanophenyl)-N-methyl-3-(1-methyl-1H-pyrazol-4-yl)imidazo[1,2-a]pyrazine-6-carboxamide; N-methyl-N-[(6-methylpyridin-2-yl)methyl]-3-[4-(trifluoromethyl)phenyl]imidazo[1,2-a]pyrazine-6-carboxamide; 3-(3-cyanophenyl)-N-(4-cyanophenyl)-N-methylimidazo[1,2-a]pyrazine-6-carboxamide; N-[(4-fluorophenyl)methyl]-N-methyl-3-[4-(trifluoromethyl)phenyl]imidazo[1,2-a]pyrazine-6-carboxamide; N-(4-cyanophenyl)-N-methyl-3-[4-(pyrrolidin-2-yl)phenyl]imidazo[1,2-a]pyrazine-6-carboxamide; N-methyl-N-(pyridin-3-ylmethyl)-3-[4-(trifluoromethyl)phenyl]imidazo[1,2-a]pyrazine-6-carboxamide; N-(4-cyanophenyl)-N-methyl-3-(pyridin-3-yl)imidazo[1,2-a]pyrazine-6-carboxamide; 3-(5-chloropyridin-2-yl)-N-(4-cyanophenyl)-N-methylimidazo[1,2-a]pyrazine-6-carboxamide; N-(4-cyanophenyl)-3-[4-(dimethylsulfamoyl)phenyl]-N-methylimidazo[1,2-a]pyrazine-6-carboxamide; N-(4-cyanophenyl)-N-methyl-3-[2-(4-methylpiperidin-1-yl)-1,3-thiazol-4-yl]imidazo[1,2-a]pyrazine-6-carboxamide; N-(4-cyanophenyl)-N-methyl-3-(3-methylphenyl)imidazo[1,2-a]pyrazine-6-carboxamide; N-(4-cyanophenyl)-N-methyl-3-(1H-pyrazol-4-yl)imidazo[1,2-a]pyrazine-6-carboxamide; N-(4-cyanophenyl)-N-methyl-3-(1-methyl-2,3-dihydro-1H-indol-5-yl)imidazo[1,2-a]pyrazine-6-carboxamide; 3-(2,1,3-benzoxadiazol-5-yl)-N-(4-cyanophenyl)-N-methylimidazo[1,2-a]pyrazine-6-carboxamide; N-(4-cyanophenyl)-N-methyl-3-{3-methyl-3H-imidazo[4,5-b]pyridin-6-yl}imidazo[1,2-a]pyrazine-6-carboxamide; N-(4-cyanophenyl)-N-methyl-3-(2-oxo-2,3-dihydro-1H-indol-5-yl)imidazo[1,2-a]pyrazine-6-carboxamide; N-(4-methanesulfonylphenyl)-N-methyl-3-(1-methyl-1H-indazol-5-yl)imidazo[1,2-a]pyrazine-6-carboxamide; N-methyl-N-{4-[3-(trifluoromethyl)-1H-pyrazol-1-yl]phenyl}-3-[4-(trifluoromethyl)phenyl]imidazo[1,2-a]pyrazine-6-carboxamide; N-(4-cyanophenyl)-3-{4-[(dimethylamino)methyl]phenyl}-N-methylimidazo[1,2-a]pyrazine-6-carboxamide; N-(4-cyanophenyl)-N-methyl-3-[4-(1-methyl-1H-pyrazol-5-yl)phenyl]imidazo[1,2-a]pyrazine-6-carboxamide; 3-[4-(difluoromethoxy)phenyl]-N-(4-methanesulfonylphenyl)-N-methylimidazo[1,2-a]pyrazine-6-carboxamide; 4-[3-(4-carbamoylphenyl)imidazo[1,2-a]pyrazin-6-yl]benzamide; N-(4-cyanophenyl)-N-methyl-3-[5-(2-oxopyrrolidin-1-yl)pyrazin-2-yl]imidazo[1,2-a]pyrazine-6-carboxamide; 3-{4-[(2S)-2-amino-2-cyclohexylacetamido]phenyl}-N-(4-cyanophenyl)-N-methylimidazo[1,2-a]pyrazine-6-carboxamide; 4-[methyl({3-[4-(trifluoromethyl)phenyl]imidazo[1,2-a]pyrazin-6-yl}methyl)amino]benzonitrile; N-(4-cyanophenyl)-3-[4-(difluoromethoxy)phenyl]-N-methylimidazo[1,2-a]pyrazine-6-carboxamide; N-(4-bromophenyl)-N-methyl-3-[4-(trifluoromethyl)phenyl]imidazo[1,2-a]pyrazine-6-carboxamide; N-(6-chloropyridin-3-yl)-N-methyl-3-[4-(piperidin-1-yl)phenyl]imidazo[1,2-a]pyrazine-6-carboxamide; N-(4-chlorophenyl)-N-methyl-3-[4-(trifluoromethyl)phenyl]imidazo[1,2-a]pyrazine-6-carboxamide; 6-chloro-1-({3-[4-(trifluoromethyl)phenyl]imidazo[1,2-a]pyrazin-6-yl}carbonyl)-1,2,3,4-tetrahydroquinoline; N-(4-chlorophenyl)-3-(4-methoxyphenyl)-N-methylimidazo[1,2-a]pyrazine-6-carboxamide; N-(2-chloro-5-methylpyridin-3-yl)-N-methyl-3-[4-(trifluoromethyl)phenyl]imidazo[1,2-a]pyrazine-6-carboxamide; N-(3-fluoropyridin-2-yl)-N-methyl-3-[4-(trifluoromethyl)phenyl]imidazo[1,2-a]pyrazine-6-carboxamide; 4-(6-{[(4-fluorophenyl)(methyl)amino]methyl}imidazo[1,2-a]pyrazin-3-yl)benzamide; 4-(6-{[(4-cyanophenyl)(methyl)amino]methyl}imidazo[1,2-a]pyrazin-3-yl)benzamide; N-(6-chloropyridin-3-yl)-N-methyl-3-[4-(1H-pyrazol-5-yl)phenyl]imidazo[1,2-a]pyrazine-6-carboxamide; methyl N-[4-(6-{[(4-cyanophenyl)(methyl)amino]methyl}imidazo[1,2-a]pyrazin-3-yl)phenyl]carbamate N-[5-(6-{[(4-cyanophenyl)(methyl)amino]methyl}imidazo[1,2-a]pyrazin-3-yl)pyridin-2-yl]acetamide; 3-[4-(5-amino-1,3,4-oxadiazol-2-yl)phenyl]-N-(4-fluorophenyl)-N-methylimidazo[1,2-a]pyrazine-6-carboxamide; N-(4-chlorophenyl)-N-methyl-3-[4-(methylcarbamoyl)phenyl]imidazo[1,2-a]pyrazine-6-carboxamide; 4-{6-[(6-fluoro-2,2-dimethyl-1,2,3,4-tetrahydroquinolin-1-yl)carbonyl]imidazo[1,2-a]pyrazin-3-yl}benzamide; 6-fluoro-2,2-dimethyl-1-({3-[4-(trifluoromethyl)phenyl]imidazo[1,2-a]pyrazin-6-yl}carbonyl)-1,2,3,4-tetrahydroquinoline; 6-chloro-N-methyl-N-({3-[4-(trifluoromethyl)phenyl]imidazo[1,2-a]pyrazin-6-yl}methyl)pyridin-3-amine; 3-(4-cyanophenyl)-N-(4-methanesulfonylphenyl)-N-methylimidazo[1,2-a]pyrazine-6-carboxamide; 4-(6-{[methyl(5-methylpyridin-2-yl)amino]methyl}imidazo[1,2-a]pyrazin-3-yl)benzamide; 6-fluoro-1-({3-[4-(trifluoromethyl)phenyl]imidazo[1,2-a]pyrazin-6-yl}methyl)-1,2,3,4-tetrahydroquinoline; N,5-dimethyl-N-({3-[4-(trifluoromethyl)phenyl]imidazo[1,2-a]pyrazin-6-yl}methyl)pyridin-3-amine; N-(4-cyanophenyl)-3-(6-acetamidopyridin-3-yl)-N-methylimidazo[1,2-a]pyrazine-6-carboxamide; N-(6-chloropyridin-3-yl)-3-[4-(1,1-difluoroethyl)phenyl]-N-methylimidazo[1,2-a]pyrazine-6-carboxamide; N-(4-cyanophenyl)-N-(2-methoxyethyl)-3-[4-(trifluoromethyl)phenyl]imidazo[1,2-a]pyrazine-6-carboxamide; N-methyl-3-[4-(trifluoromethyl)phenyl]-N-[6-(trifluoromethyl)pyridin-3-yl]imidazo[1,2-a]pyrazine-6-carboxamide; N-(4-cyanophenyl)-N-methyl-3-[4-(1,3,4-oxadiazol-2-yl)phenyl]imidazo[1,2-a]pyrazine-6-carboxamide; N-({3-[4-(difluoromethyl)phenyl]imidazo[1,2-a]pyrazin-6-yl}methyl)-N,5-dimethylpyridin-2-amine; N-[5-(6-{[methyl(5-methylpyridin-2-yl)amino]methyl}imidazo[1,2-a]pyrazin-3-yl)pyridin-2-yl]acetamide; N-(4-cyanophenyl)-N-methyl-3-{1H-pyrrolo[2,3-b]pyridin-5-yl}imidazo[1,2-a]pyrazine-6-carboxamide; N-(4-fluorophenyl)-N-methyl-3-[4-(methylcarbamoyl)phenyl]imidazo[1,2-a]pyrazine-6-carboxamide; methyl N-(4-{6-[(6-chloropyridin-3-yl)(methyl)carbamoyl]imidazo[1,2-a]pyrazin-3-yl}phenyl)carbamate; N-(4-cyanophenyl)-N-methyl-3-[6-(methylamino)pyridin-3-yl]imidazo[1,2-a]pyrazine-6-carboxamide; 3-[4-(2-aminoacetamido)phenyl]-N-(4-cyanophenyl)-N-methylimidazo[1,2-a]pyrazine-6-carboxamide; 3-[4-(5-amino-1,3,4-oxadiazol-2-yl)phenyl]-N-(4-cyanophenyl)-N-methylimidazo[1,2-a]pyrazine-6-carboxamide; N-methyl-N-(pyridin-4-yl)-3-[4-(trifluoromethyl)phenyl]imidazo[1,2-a]pyrazine-6-carboxamide; N-(2-methoxy-5-methylpyridin-3-yl)-N-methyl-3-[4-(trifluoromethyl)phenyl]imidazo[1,2-a]pyrazine-6-carboxamide; N-methyl-N-(5-methylpyridin-3-yl)-3-[4-(trifluoromethyl)phenyl]imidazo[1,2-a]pyrazine-6-carboxamide; N-(2-methoxy-5-methylpyridin-3-yl)-N-methyl-3-[4-(trifluoromethyl)phenyl]imidazo[1,2-a]pyrazine-6-carboxamide; ethyl 6-[(6-chloropyridin-3-yl)(methyl)carbamoyl]imidazo[1,2-a]pyrazine-3-carboxylate; ethyl N-(4-{6-[(4-cyanophenyl)(methyl)carbamoyl]imidazo[1,2-a]pyrazin-3-yl}phenyl)carbamate; 3-(4-{[(1S,2R)-2-aminocyclopentane]amido}phenyl)-N-(4-cyanophenyl)-N-methylimidazo[1,2-a]pyrazine-6-carboxamide; 3-{4-[(3S)-3-amino-4-methylpentanamido]phenyl}-N-(4-cyanophenyl)-N-methylimidazo[1,2-a]pyrazine-6-carboxamide; N-(4-fluorophenyl)-N-methyl-3-[4-(1H-pyrazol-5-yl)phenyl]imidazo[1,2-a]pyrazine-6-carboxamide; N-(3,4-difluorophenyl)-N-methyl-3-[4-(1H-pyrazol-5-yl)phenyl]imidazo[1,2-a]pyrazine-6-carboxamide; 4-chloro-N-methyl-N-{3-[4-(trifluoromethyl)phenyl]imidazo[1,2-a]pyrazin-6-yl}benzamide; methyl N-(5-{6-[(4-cyanophenyl)(methyl)carbamoyl]imidazo[1,2-a]pyrazin-3-yl}pyridin-2-yl)carbamate; Propan-2-yl N-(4-{6-[(4-cyanophenyl)(methyl)carbamoyl]imidazo[1,2-a]pyrazin-3-yl}phenyl)carbamate; N-(6-chloropyridin-3-yl)-N-methyl-3-{4-[5-(methylamino)-1,3,4-thiadiazol-2-yl]phenyl}imidazo[1,2-a]pyrazine-6-carboxamide; N,4-dimethyl-N-({3-[4-(trifluoromethyl)phenyl]imidazo[1,2-a]pyrazin-6-yl}methyl)aniline; 5-chloro-N-methyl-N-{3-[4-(trifluoromethyl)phenyl]imidazo[1,2-a]pyrazin-6-yl}pyridine-2-carboxamide; N-(6-chloropyridin-3-yl)-3-[4-(difluoromethyl)phenyl]-N-methylimidazo[1,2-a]pyrazine-6-carboxamide; 3-{4-[(2S)-2-amino-3-methylbutanamido]phenyl}-N-(4-cyanophenyl)-N-methylimidazo[1,2-a]pyrazine-6-carboxamide; 6-chloro-N-methyl-N-{3-[4-(trifluoromethyl)phenyl]imidazo[1,2-a]pyrazin-6-yl}pyridine-3-carboxamide; 4-fluoro-N-methyl-N-{3-[4-(trifluoromethyl)phenyl]imidazo[1,2-a]pyrazin-6-yl}benzamide; 3-{4-[(3R)-3-aminobutanamido]phenyl}-N-(4-cyanophenyl)-N-methylimidazo[1,2-a]pyrazine-6-carboxamide; 3-[4-(2-amino-2-cyclobutylacetamido)phenyl]-N-(4-cyanophenyl)-N-methylimidazo[1,2-a]pyrazine-6-carboxamide; 3-{4-[(2S)-2-aminopropanamido]phenyl}-N-(4-cyanophenyl)-N-methylimidazo[1,2-a]pyrazine-6-carboxamide; 3-(6-aminopyridin-3-yl)-N-(4-cyanophenyl)-N-methylimidazo[1,2-a]pyrazine-6-carboxamide; 3-[4-(cyanomethoxy)phenyl]-N-(4-cyanophenyl)-N-methylimidazo[1,2-a]pyrazine-6-carboxamide; 1-({3-[4-(trifluoromethyl)phenyl]imidazo[1,2-a]pyrazin-6-yl}carbonyl)-1,2,3,4-tetrahydroquinoline-6-carbonitrile; N-(6-chloropyridin-3-yl)-3-[2-fluoro-4-(trifluoromethyl)phenyl]-N-methylimidazo[1,2-a]pyrazine-6-carboxamide; N-methyl-4-(trifluoromethyl)-N-{3-[4-(trifluoromethyl)phenyl]imidazo[1,2-a]pyrazin-6-yl}benzamide; N-methyl-N-(6-methylpyridin-3-yl)-3-[4-(trifluoromethyl)phenyl]imidazo[1,2-a]pyrazine-6-carboxamide; 3-{4-[(2-amino-2-methylpropyl)amino]phenyl}-N-(4-cyanophenyl)-N-methylimidazo[1,2-a]pyrazine-6-carboxamide; N-(6-chloropyridin-3-yl)-3-(4-acetamidophenyl)-N-methylimidazo[1,2-a]pyrazine-6-carboxamide; N-(6-chloropyridin-3-yl)-3-(2-fluoro-4-methoxyphenyl)-N-methylimidazo[1,2-a]pyrazine-6-carboxamide; 7-fluoro-4-({3-[4-(trifluoromethyl)phenyl]imidazo[1,2-a]pyrazin-6-yl}methyl)-3,4-dihydro-2H-1,4-benzoxazine; 3-{4-[(2R)-2-aminopropanamido]phenyl}-N-(4-cyanophenyl)-N-methylimidazo[1,2-a]pyrazine-6-carboxamide; 4-cyano-N-methyl-N-{3-[4-(trifluoromethyl)phenyl]imidazo[1,2-a]pyrazin-6-yl}benzamide; N-(4-cyanophenyl)-3-{4-[2-(dimethylamino)ethoxy]phenyl}-N-methylimidazo[1,2-a]pyrazine-6-carboxamide; N-(4-cyanophenyl)-N,2-dimethyl-3-[4-(trifluoromethyl)phenyl]imidazo[1,2-a]pyrazine-6-carboxamide; 3-{4-[(2R)-2-amino-3-methylbutanamido]phenyl}-N-(4-cyanophenyl)-N-methylimidazo[1,2-a]pyrazine-6-carboxamide; N,5-dimethyl-N-({3-[4-(trifluoromethyl)phenyl]imidazo[1,2-a]pyrazin-6-yl}methyl)pyridin-2-amine; N-(6-methoxy-5-methylpyridin-3-yl)-N-methyl-3-[4-(trifluoromethyl)phenyl]imidazo[1,2-a]pyrazine-6-carboxamide; N,4-dimethyl-N-{3-[4-(trifluoromethyl)phenyl]imidazo[1,2-a]pyrazin-6-yl}benzamide; N-(6-methoxypyridin-2-yl)-N-methyl-3-[4-(trifluoromethyl)phenyl]imidazo[1,2-a]pyrazine-6-carboxamide; N-(4-cyanophenyl)-3-(4-{[2-(dimethylamino)ethyl]carbamoyl}phenyl)-N-methylimidazo[1,2-a]pyrazine-6-carboxamide; 3-{4-[(2S)-2-amino-2-cyclopropylacetamido]phenyl}-N-(4-cyanophenyl)-N-methylimidazo[1,2-a]pyrazine-6-carboxamide; N-(4-cyano-2-fluorophenyl)-N-methyl-3-[4-(trifluoromethyl)phenyl]imidazo[1,2-a]pyrazine-6-carboxamide; N,4,4-trimethyl-N-{3-[4-(trifluoromethyl)phenyl]imidazo[1,2-a]pyrazin-6-yl}piperidine-1-carboxamide; 3-[6-(2-aminoacetamido)pyridin-3-yl]-N-(4-cyanophenyl)-N-methylimidazo[1,2-a]pyrazine-6-carboxamide; N-(2-methoxypyridin-4-yl)-N-methyl-3-[4-(trifluoromethyl)phenyl]imidazo[1,2-a]pyrazine-6-carboxamide; N-(4-cyanophenyl)-N-methyl-3-(6-methylpyridin-3-yl)imidazo[1,2-a]pyrazine-6-carboxamide; N-(1-ethyl-1H-pyrazol-5-yl)-N-methyl-3-[4-(trifluoromethyl)phenyl]imidazo[1,2-a]pyrazine-6-carboxamide; N-methyl-N-(2-methylpyridin-4-yl)-3-[4-(trifluoromethyl)phenyl]imidazo[1,2-a]pyrazine-6-carboxamide; tert-butyl N-{2-[(4-{6-[(4-cyanophenyl)(methyl)carbamoyl]imidazo[1,2-a]pyrazin-3-yl}phenyl)amino]-2-methylpropyl}carbamate; N-(6-chloropyridin-3-yl)-3-(1H-indol-5-yl)-N-methylimidazo[1,2-a]pyrazine-6-carboxamide; 3-(4-methoxy-2-methylphenyl)-N-[6-(4-methoxy-2-methylphenyl)pyridin-3-yl]-N-methylimidazo[1,2-a]pyrazine-6-carboxamide; N-(4-methoxypyridin-3-yl)-N-methyl-3-[4-(trifluoromethyl)phenyl]imidazo[1,2-a]pyrazine-6-carboxamide; N-(6-chloro-5-methylpyridin-3-yl)-N-methyl-3-[4-(trifluoromethyl)phenyl]imidazo[1,2-a]pyrazine-6-carboxamide; 7-fluoro-4-({3-[4-(trifluoromethyl)phenyl]imidazo[1,2-a]pyrazin-6-yl}carbonyl)-3,4-dihydro-2H-1,4-benzoxazine; -({3-[4-(trifluoromethyl)phenyl]imidazo[1,2-a]pyrazin-6-yl}carbonyl)-3,4-dihydro-2H-1,4-benzoxazine; 3-(4-{[1-(aminomethyl)cyclopropane]amido}phenyl)-N-(4-cyanophenyl)-N-methylimidazo[1,2-a]pyrazine-6-carboxamide; N-methyl-6-(trifluoromethyl)-N-{3-[4-(trifluoromethyl)phenyl]imidazo[1,2-a]pyrazin-6-yl}pyridine-3-carboxamide; N-methyl-N-(4-methylpyridin-3-yl)-3-[4-(trifluoromethyl)phenyl]imidazo[1,2-a]pyrazine-6-carboxamide; N-(4-ethoxypyridin-2-yl)-N-methyl-3-[4-(trifluoromethyl)phenyl]imidazo[1,2-a]pyrazine-6-carboxamide; 4-methoxy-N-methyl-N-{3-[4-(trifluoromethyl)phenyl]imidazo[1,2-a]pyrazin-6-yl}benzamide; N-(6-chloropyridin-3-yl)-N-methyl-3-{1H-pyrrolo[2,3-b]pyridin-5-yl}imidazo[1,2-a]pyrazine-6-carboxamide; N-methyl-N-[4-(1H-1,2,4-triazol-1-yl)phenyl]-3-[4-(trifluoromethyl)phenyl]imidazo[1,2-a]pyrazine-6-carboxamide; N-(4-cyanophenyl)-3-(4-methanesulfonamidophenyl)-N-methylimidazo[1,2-a]pyrazine-6-carboxamide; 4-({3-[4-(trifluoromethyl)phenyl]imidazo[1,2-a]pyrazin-6-yl}carbonyl)-3,4-dihydro-2H-1,4-benzoxazine-7-carbonitrile; 6-methanesulfonyl-1-({3-[4-(trifluoromethyl)phenyl]imidazo[1,2-a]pyrazin-6-yl}carbonyl)-1,2,3,4-tetrahydroquinoline; N-(5-methanesulfonylpyridin-2-yl)-N-methyl-3-[4-(trifluoromethyl)phenyl]imidazo[1,2-a]pyrazine-6-carboxamide; methyl N-[4-(6-{[methyl(5-methylpyridin-2-yl)amino]methyl}imidazo[1,2-a]pyrazin-3-yl)phenyl]carbamate; 3-(1H-indol-2-yl)-N-[6-(1H-indol-2-yl)pyridin-3-yl]-N-methylimidazo[1,2-a]pyrazine-6-carboxamide; N-(6-chloropyridin-3-yl)-N-methyl-3-(3-methyl-1H-indazol-5-yl)imidazo[1,2-a]pyrazine-6-carboxamide; N-methyl-N-[5-(morpholin-4-yl)pyridin-2-yl]-3-[4-(trifluoromethyl)phenyl]imidazo[1,2-a]pyrazine-6-carboxamide; 4-fluoro-N-methyl-N-{3-[4-(trifluoromethyl)phenyl]imidazo[1,2-a]pyrazin-6-yl}piperidine-1-carboxamide; N-(4-cyanophenyl)-3-(6-methanesulfonamidopyridin-3-yl)-N-methylimidazo[1,2-a]pyrazine-6-carboxamide; 3-{4-[(1-amino-2-methylpropan-2-yl)amino]phenyl}-N-(4-cyanophenyl)-N-methylimidazo[1,2-a]pyrazine-6-carboxamide; 3-(1-benzofuran-5-yl)-N-(6-chloropyridin-3-yl)-N-methylimidazo[1,2-a]pyrazine-6-carboxamide; N-methyl-N-(pyridin-3-yl)-3-[4-(trifluoromethyl)phenyl]imidazo[1,2-a]pyrazine-6-carboxamide; N-methyl-N-(5-methylpyrazin-2-yl)-3-[4-(trifluoromethyl)phenyl]imidazo[1,2-a]pyrazine-6-carboxamide; N-methyl-N-(5-methylpyrazin-2-yl)-3-[4-(trifluoromethyl)phenyl]imidazo[1,2-a]pyrazine-6-carboxamide; N-methyl-3-[4-(trifluoromethyl)phenyl]-N-[2-(trifluoromethyl)pyridin-4-yl]imidazo[1,2-a]pyrazine-6-carboxamide; N-(6-chloropyridin-3-yl)-N-methyl-3-[4-(1H-pyrazol-1-yl)phenyl]imidazo[1,2-a]pyrazine-6-carboxamide; 5-{6-[(4-cyanophenyl)(methyl)carbamoyl]imidazo[1,2-a]pyrazin-3-yl}-N-methylpyridine-2-carboxamide; N-(6-chloropyridin-3-yl)-3-[3-fluoro-4-(trifluoromethyl)phenyl]-N-methylimidazo[1,2-a]pyrazine-6-carboxamide; N-(4-cyanophenyl)-3-(2-methoxypyrimidin-5-yl)-N-methylimidazo[1,2-a]pyrazine-6-carboxamide; N-(4-ethylpyridin-2-yl)-3-[4-(trifluoromethyl)phenyl]imidazo[1,2-a]pyrazine-6-carboxamide; N-(4-cyanophenyl)-3-(6-methoxypyrazin-2-yl)-N-methylimidazo[1,2-a]pyrazine-6-carboxamide; N-(6-chloropyridin-3-yl)-3-(6-methoxypyridin-3-yl)-N-methylimidazo[1,2-a]pyrazine-6-carboxamide; N-methyl-3-[4-(trifluoromethyl)phenyl]-N-[5-(trifluoromethyl)pyridin-2-yl]imidazo[1,2-a]pyrazine-6-carboxamide; N-methyl-3-[4-(trifluoromethyl)phenyl]-N-[5-(trifluoromethyl)pyridin-2-yl]imidazo[1,2-a]pyrazine-6-carboxamide; N-(4-cyanophenyl)-N-methyl-3-[4-(3-methyl-1,2,4-oxadiazol-5-yl)phenyl]imidazo[1,2-a]pyrazine-6-carboxamide; N-(6-chloropyridin-3-yl)-N-methyl-3-[4-(morpholin-4-yl)phenyl]imidazo[1,2-a]pyrazine-6-carboxamide; N,3-bis(4-cyanophenyl)-N-methyl-2-(trifluoromethyl)imidazo[1,2-a]pyrazine-6-carboxamide; N-(2,6-dichloropyridin-3-yl)-N-methyl-3-[4-(trifluoromethyl)phenyl]imidazo[1,2-a]pyrazine-6-carboxamide; N-(6-chloropyridin-3-yl)-3-[4-(cyclopropylcarbamoyl)phenyl]-N-methylimidazo[1,2-a]pyrazine-6-carboxamide; N-(4-cyanophenyl)-3-[2-(dimethylamino)pyrimidin-5-yl]-N-methylimidazo[1,2-a]pyrazine-6-carboxamide; N-methyl-N-(6-methylpyridin-2-yl)-3-[4-(trifluoromethyl)phenyl]imidazo[1,2-a]pyrazine-6-carboxamide; N-[2-(difluoromethoxy)phenyl]-N-methyl-3-[4-(trifluoromethyl)phenyl]imidazo[1,2-a]pyrazine-6-carboxamide; 4-cyano-N-methyl-N-{3-[4-(trifluoromethyl)phenyl]imidazo[1,2-a]pyrazin-6-yl}benzene-1-sulfonamide; N-methyl-3-[4-(trifluoromethyl)phenyl]-N-[4-(trifluoromethyl)pyridin-2-yl]imidazo[1,2-a]pyrazine-6-carboxamide; N-(6-chloropyridin-3-yl)-3-(1H-indazol-5-yl)-N-methylimidazo[1,2-a]pyrazine-6-carboxamide; 2,2-dimethyl-4-({3-[4-(trifluoromethyl)phenyl]imidazo[1,2-a]pyrazin-6-yl}carbonyl)-3,4-dihydro-2H-1,4-benzoxazine-7-carbonitrile; N-(4-cyanophenyl)-3-(6-fluoropyridin-2-yl)-N-methylimidazo[1,2-a]pyrazine-6-carboxamide; 3-[4-(1-carbamoyl-1-methylethoxy)phenyl]-N-(4-cyanophenyl)-N-methylimidazo[1,2-a]pyrazine-6-carboxamide; N-(5-methoxypyridin-2-yl)-N-methyl-3-[4-(trifluoromethyl)phenyl]imidazo[1,2-a]pyrazine-6-carboxamide; 3-(3-chloropyridin-2-yl)-N-(4-cyanophenyl)-N-methylimidazo[1,2-a]pyrazine-6-carboxamide; N-(5-chloropyridin-3-yl)-N-methyl-3-[4-(trifluoromethyl)phenyl]imidazo[1,2-a]pyrazine-6-carboxamide; N-methyl-N-(1,3-thiazol-2-yl)-3-[4-(trifluoromethyl)phenyl]imidazo[1,2-a]pyrazine-6-carboxamide; N-(6-chloropyridin-3-yl)-3-[6-(dimethylamino)pyridin-3-yl]-N-methylimidazo[1,2-a]pyrazine-6-carboxamide; N-(4-methoxypyridin-2-yl)-N-methyl-3-[4-(trifluoromethyl)phenyl]imidazo[1,2-a]pyrazine-6-carboxamide; N-{6-[(2S)-2-(methoxymethyl)pyrrolidin-1-yl]pyridin-3-yl}-N-methyl-3-[4-(trifluoromethyl)phenyl]imidazo[1,2-a]pyrazine-6-carboxamide; N-(6-chloropyridin-3-yl)-3-(2,3-dihydro-1,4-benzodioxin-6-yl)-N-methylimidazo[1,2-a]pyrazine-6-carboxamide; 3-(4-acetamidophenyl)-N-methyl-N-(5-methylpyridin-2-yl)imidazo[1,2-a]pyrazine-6-carboxamide; N-[(4-cyanophenyl)methyl]-3-[4-(trifluoromethyl)phenyl]imidazo[1,2-a]pyrazine-6-carboxamide; N-(4-cyanophenyl)-N-methyl-3-[4-(propane-2-sulfonamido)phenyl]imidazo[1,2-a]pyrazine-6-carboxamide; N-(6-chloropyridin-3-yl)-N-methyl-3-[2-(propan-2-yl)-1,3-thiazol-4-yl]imidazo[1,2-a]pyrazine-6-carboxamide; N-methyl-N-[6-(1H-1,2,4-triazol-1-yl)pyridin-3-yl]-3-[4-(trifluoromethyl)phenyl]imidazo[1,2-a]pyrazine-6-carboxamide; N-methyl-4-(trifluoromethoxy)-N-{3-[4-(trifluoromethyl)phenyl]imidazo[1,2-a]pyrazin-6-yl}benzamide; N-(6-chloropyridin-3-yl)-N-methyl-3-[6-(5-methyl-1,3,4-oxadiazol-2-yl)pyridin-3-yl]imidazo[1,2-a]pyrazine-6-carboxamide; N-(1-ethyl-1H-pyrazol-5-yl)-3-[4-(trifluoromethyl)phenyl]imidazo[1,2-a]pyrazine-6-carboxamide; 3-(4-cyanophenyl)-N-methyl-N-[4-(trifluoromethyl)phenyl]imidazo[1,2-a]pyrazine-6-carboxamide; N-benzyl-N-methyl-3-[4-(trifluoromethyl)phenyl]imidazo[1,2-a]pyrazine-6-carboxamide; 2-C-3-[4-(trifluoromethyl)phenyl]imidazo[1,2-a]pyrazine-6-pyridine-2,5-dicarboxamide; 3-[4-(2-aminoethoxy)phenyl]-N-(4-cyanophenyl)-N-methylimidazo[1,2-a]pyrazine-6-carboxamide; 3-(6-acetamidopyridin-3-yl)-N-methyl-N-(5-methylpyridin-2-yl)imidazo[1,2-a]pyrazine-6-carboxamide; 4-({3-[4-(trifluoromethyl)phenyl]imidazo[1,2-a]pyrazin-6-yl}carbonyl)morpholine; N-[4-(aminomethyl)phenyl]-N-methyl-3-[4-(trifluoromethyl)phenyl]imidazo[1,2-a]pyrazine-6-carboxamide; N-(4-cyanophenyl)-2-methyl-3-[4-(trifluoromethyl)phenyl]imidazo[1,2-a]pyrazine-6-carboxamide; N,3-bis(4-cyanophenyl)-N-methylimidazo[1,2-a]pyrazine-6-carboxamide; N-(4-cyanophenyl)-N-methyl-3-(2-methylpyridin-4-yl)imidazo[1,2-a]pyrazine-6-carboxamide; N-(6-chloropyridin-3-yl)-N-methyl-3-(2-oxo-2,3-dihydro-1H-indol-5-yl)imidazo[1,2-a]pyrazine-6-carboxamide; 3-(2-aminopyrimidin-5-yl)-N-(4-cyanophenyl)-N-methylimidazo[1,2-a]pyrazine-6-carboxamide; N-(6-chloropyridin-3-yl)-3-ethenyl-N-methylimidazo[1,2-a]pyrazine-6-carboxamide; N-(5-cyclobutylpyridin-2-yl)-N-methyl-3-[4-(trifluoromethyl)phenyl]imidazo[1,2-a]pyrazine-6-carboxamide; N-{[4-(morpholin-4-yl)phenyl]methyl}-3-[4-(trifluoromethyl)phenyl]imidazo[1,2-a]pyrazine-6-carboxamide; N-(6-tert-butylpyridin-3-yl)-N-methyl-3-[4-(trifluoromethyl)phenyl]imidazo[1,2-a]pyrazine-6-carboxamide; N-(4-cyanophenyl)-N-methyl-3-(pyridin-4-yl)imidazo[1,2-a]pyrazine-6-carboxamide; N-(2-aminoethyl)-N-(4-cyanophenyl)-3-[4-(trifluoromethyl)phenyl]imidazo[1,2-a]pyrazine-6-carboxamide; N-(6-chloropyridin-3-yl)-3-(1H-indol-2-yl)-N-methylimidazo[1,2-a]pyrazine-6-carboxamide; N-(6-chloropyridin-3-yl)-3-{4-[(dimethylamino)methyl]phenyl}-N-methylimidazo[1,2-a]pyrazine-6-carboxamide; N-(4-cyano-1H-imidazol-5-yl)-3-[4-(trifluoromethyl)phenyl]imidazo[1,2-a]pyrazine-6-carboxamide; N-methyl-N-(3-methylpyridin-2-yl)-3-[4-(trifluoromethyl)phenyl]imidazo[1,2-a]pyrazine-6-carboxamide; N-(4-cyanophenyl)-N-methyl-3-{1-methyl-1H-pyrazolo[3,4-b]pyridin-5-yl}imidazo[1,2-a]pyrazine-6-carboxamide; 4-({3-[4-(trifluoromethyl)phenyl]imidazo[1,2-a]pyrazin-6-yl}methyl)morpholine; 5-{6-[(4-cyanophenyl)(methyl)carbamoyl]imidazo[1,2-a]pyrazin-3-yl}pyridine-2-carboxamide; N-[6-(dimethylamino)pyridin-3-yl]-N-methyl-3-[4-(trifluoromethyl)phenyl]imidazo[1,2-a]pyrazine-6-carboxamide; N-(6-methoxypyridazin-3-yl)-N-methyl-3-[4-(trifluoromethyl)phenyl]imidazo[1,2-a]pyrazine-6-carboxamide; 3-(3-amino-1H-indazol-5-yl)-N-(4-cyanophenyl)-N-methylimidazo[1,2-a]pyrazine-6-carboxamide; 1-({3-[4-(trifluoromethyl)phenyl]imidazo[1,2-a]pyrazin-6-yl}carbonyl)piperidine-3-carboxamide; N-(6-chloropyridin-3-yl)-N-methyl-3-[4-(methylamino)phenyl]imidazo[1,2-a]pyrazine-6-carboxamide; N-(2-methoxypyrimidin-5-yl)-N-methyl-3-[4-(trifluoromethyl)phenyl]imidazo[1,2-a]pyrazine-6-carboxamide; N-(4-cyanophenyl)-N-methyl-3-(6-{[2-(morpholin-4-yl)ethyl]amino}pyridin-3-yl)imidazo[1,2-a]pyrazine-6-carboxamide; N-(4-cyanophenyl)-3-(6-cyanopyridin-3-yl)-N-methylimidazo[1,2-a]pyrazine-6-carboxamide; 3-{4-[(1-amino-2-methylpropan-2-yl)oxy]phenyl}-N-(4-cyanophenyl)-N-methylimidazo[1,2-a]pyrazine-6-carboxamide; 6-N-(6-chloropyridin-3-yl)-6-N-methyl-3-N-[4-(trifluoromethyl)phenyl]imidazo[1,2-a]pyrazine-3,6-dicarboxamide; 5-C-3-[4-(trifluoromethyl)phenyl]imidazo[1,2-a]pyrazine-6-1H-imidazole-4,5-dicarboxamide; N-[1-(4-chlorophenyl)ethyl]-N-methyl-3-[4-(trifluoromethyl)phenyl]imidazo[1,2-a]pyrazine-6-carboxamide; N-{4-[(1,3-thiazol-2-yl)sulfamoyl]phenyl}-3-[4-(trifluoromethyl)phenyl]imidazo[1,2-a]pyrazine-6-carboxamide; N-(6-chloropyridin-3-yl)-N-methyl-3-[6-(methylamino)pyridin-3-yl]imidazo[1,2-a]pyrazine-6-carboxamide; 3-[4-(2-amino-2-methylpropanamido)phenyl]-N-(4-cyanophenyl)-N-methylimidazo[1,2-a]pyrazine-6-carboxamide; 3-[4-(2-aminoethoxy)phenyl]-N-methyl-N-(5-methylpyridin-2-yl)imidazo[1,2-a]pyrazine-6-carboxamide; N-(6-chloropyridin-3-yl)-N-methyl-3-[4-(1,3,4-oxadiazol-2-yl)phenyl]imidazo[1,2-a]pyrazine-6-carboxamide; 6-fluoro-1-({8-methyl-3-[4-(trifluoromethyl)phenyl]imidazo[1,2-a]pyrazin-6-yl}carbonyl)-1,2,3,4-tetrahydroquinoline; N-(5-cyclopentylpyridin-2-yl)-N-methyl-3-[4-(trifluoromethyl)phenyl]imidazo[1,2-a]pyrazine-6-carboxamide; N-(6-chloropyridin-3-yl)-N-methyl-3-[6-(trifluoromethyl)pyridin-3-yl]imidazo[1,2-a]pyrazine-6-carboxamide; N-[2-(4-fluorophenyl)propan-2-yl]-3-[4-(trifluoromethyl)phenyl]imidazo[1,2-a]pyrazine-6-carboxamide; methyl N-(5-{6-[methyl(5-methylpyridin-2-yl)carbamoyl]imidazo[1,2-a]pyrazin-3-yl}pyridin-2-yl)carbamate; N-methyl-N-{[1,2,4]triazolo[4,3-a]pyridin-5-yl}-3-[4-(trifluoromethyl)phenyl]imidazo[1,2-a]pyrazine-6-carboxamide; 4-tert-butyl-N-methyl-N-{3-[4-(trifluoromethyl)phenyl]imidazo[1,2-a]pyrazin-6-yl}benzamide; N-(6-chloropyridin-3-yl)-3-[3-(dimethylamino)phenyl]-N-methylimidazo[1,2-a]pyrazine-6-carboxamide; N-(6-chloropyridin-3-yl)-3-(4-methanesulfonamidophenyl)-N-methylimidazo[1,2-a]pyrazine-6-carboxamide; N-methyl-N-(2-methylpyridin-3-yl)-3-[4-(trifluoromethyl)phenyl]imidazo[1,2-a]pyrazine-6-carboxamide; N-methyl-N-(5-methylpyrimidin-2-yl)-3-[4-(trifluoromethyl)phenyl]imidazo[1,2-a]pyrazine-6-carboxamide; N-(6-chloropyridin-3-yl)-3-(4-fluorophenyl)-N-methylimidazo[1,2-a]pyrazine-6-carboxamide; N-methyl-N-(4-phenyl-1,3-thiazol-2-yl)-3-[4-(trifluoromethyl)phenyl]imidazo[1,2-a]pyrazine-6-carboxamide; cyanophenyl)-N-methyl-3-(3-methyl-1H-indazol-5-yl)imidazo[1,2-a]pyrazine-6-carboxamide; N-(4-acetylphenyl)-N-methyl-3-[4-(trifluoromethyl)phenyl]imidazo[1,2-a]pyrazine-6-carboxamide; 1-({3-[4-(trifluoromethyl)phenyl]imidazo[1,2-a]pyrazin-6-yl}methyl)piperidin-2-one; N-(4-cyanophenyl)-3-[4-(dimethylcarbamoyl)phenyl]-N-methylimidazo[1,2-a]pyrazine-6-carboxamide; N-(4-cyanophenyl)-3-(1H-indol-5-yl)-N-methylimidazo[1,2-a]pyrazine-6-carboxamide; N-(6-chloropyridin-3-yl)-3-[4-(difluoromethoxy)phenyl]-N-methylimidazo[1,2-a]pyrazine-6-carboxamide; N-(4-fluoro-2-methoxyphenyl)-N-methyl-3-[4 (trifluoromethyl)phenyl]imidazo[1,2-a]pyrazine; N-(3,4-difluorophenyl)-3-(4-methoxyphenyl)-N-methylimidazo[1,2-a]pyrazine-6-carboxamide; N-(4-chlorophenyl)-N-methyl-3-(4-methylphenyl)imidazo[1,2-a]pyrazine-6-carboxamide; 3-[4-(difluoromethoxy)phenyl]-N-(4-fluorophenyl)-N-methylimidazo[1,2-a]pyrazine-6-carboxamide; N-(5-bromopyrimidin-2-yl)-N-methyl-3-[4-(trifluoromethyl)phenyl]imidazo[1,2-a]pyrazine-6-carboxamide; N-(5-chloropyridin-2-yl)-N-methyl-3-[4-(trifluoromethyl)phenyl]imidazo[1,2-a]pyrazine-6-carboxamide; N-methyl-N-[4-(propan-2-yloxy)phenyl]-3-[4-(trifluoromethyl)phenyl]imidazo[1,2-a]pyrazine-6-carboxamide; 4-[({3-[4-(dimethylamino)phenyl]imidazo[1,2-a]pyrazin-6-yl}methyl)(methyl)amino]benzonitrile; N-(4-cyanophenyl)-3-[3-(dimethylamino)phenyl]-N-methylimidazo[1,2-a]pyrazine-6-carboxamide; 3-(6-chloropyridin-3-yl)-N-(4-cyanophenyl)-N-methylimidazo[1,2-a]pyrazine-6-carboxamide; N-(3-methoxyphenyl)-N-methyl-3-[4-(trifluoromethyl)phenyl]imidazo[1,2-a]pyrazine-6-carboxamide; N-(4-cyanophenyl)-3-(4-methoxy-2-methylphenyl)-N-methylimidazo[1,2-a]pyrazine-6-carboxamide; N-(7-chloro-1,3-benzothiazol-2-yl)-3-[4-(trifluoromethyl)phenyl]imidazo[1,2-a]pyrazine-6-carboxamide; 4-fluoro-N-methyl-N-(3-{4-[5-(methylamino)-1,3,4-thiadiazol-2-yl]phenyl}imidazo[1,2-a]pyrazin-6-yl)benzamide; 3-{6-[(6-fluoro-1,2,3,4-tetrahydroquinolin-1-yl)carbonyl]imidazo[1,2-a]pyrazin-3-yl}-N,N-dimethylaniline; N,3-bis[4-(trifluoromethyl)phenyl]imidazo[1,2-a]pyrazine-6-carboxamide; N-(4-cyanophenyl)-N-methyl-3-{4-[5-(methylamino)-1,3,4-oxadiazol-2-yl]phenyl}imidazo[1,2-a]pyrazine-6-carboxamide; 4-{6-[(7-fluoro-3,3-dimethyl-3,4-dihydro-2H-1,4-benzoxazin-4-yl)carbonyl]imidazo[1,2-a]pyrazin-3-yl}benzamide; 4-{6-[(6-fluoro-1,2,3,4-tetrahydroquinolin-1-yl)carbonyl]imidazo[1,2-a]pyrazin-3-yl}benzamide; N-(4-fluorophenyl)-N-methyl-3-[4-(1,3,4-thiadiazol-2-yl)phenyl]imidazo[1,2-a]pyrazine-6-carboxamide; 4-{6-[(6-fluoro-1,2,3,4-tetrahydroquinolin-1-yl)carbonyl]imidazo[1,2-a]pyrazin-3-yl}-N-methylbenzamide; N-(4-{6-[(7-fluoro-3,3-dimethyl-3,4-dihydro-2H-1,4-benzoxazin-4-yl)carbonyl]imidazo[1,2-a]pyrazin-3-yl}phenyl)acetamide; N-methyl-4-{6-[N-methyl(4-fluorobenzene)amido]imidazo[1,2-a]pyrazin-3-yl}benzamide; N-methyl-N-(4-sulfamoylphenyl)-3-[4-(trifluoromethyl)phenyl]imidazo[1,2-a]pyrazine-6-carboxamide; 4-{6-[(7-fluoro-3,4-dihydro-2H-1,4-benzoxazin-4-yl)carbonyl]imidazo[1,2-a]pyrazin-3-yl}benzamide; 4-{6-[N-methyl(4-fluorobenzene)amido]imidazo[1,2-a]pyrazin-3-yl}benzamide; 4-fluoro-N-methyl-N-(3-{1H-pyrrolo[2,3-b]pyridin-5-yl}imidazo[1,2-a]pyrazin-6-yl)benzamide; 1-{[3-(2,3-dihydro-1-benzofuran-5-yl)imidazo[1,2-a]pyrazin-6-yl]carbonyl}-6-fluoro-1,2,3,4-tetrahydroquinoline; 3-(4-carbamoylphenyl)-N-methyl-N-(5-methylpyridin-2-yl)imidazo[1,2-a]pyrazine-6-carboxamide; 4-{6-[(7-fluoro-3,4-dihydro-2H-1,4-benzoxazin-4-yl)carbonyl]imidazo[1,2-a]pyrazin-3-yl}-N-methylbenzamide; 6-fluoro-1-{[3-(3-methanesulfonylphenyl)imidazo[1,2-a]pyrazin-6-yl]carbonyl}-1,2,3,4-tetrahydroquinoline; 3-[6-(4-acetylpiperazin-1-yl)pyridin-3-yl]-N-(4-cyanophenyl)-N-methylimidazo[1,2-a]pyrazine-6-carboxamide; 6-fluoro-1-{[3-(4-methanesulfonylphenyl)imidazo[1,2-a]pyrazin-6-yl]carbonyl}-1,2,3,4-tetrahydroquinoline; 1-{[3-(1-ethyl-1H-pyrazol-4-yl)imidazo[1,2-a]pyrazin-6-yl]carbonyl}-6-fluoro-1,2,3,4-tetrahydroquinoline; N-(4-cyanophenyl)-N-methyl-3-[6-(piperazin-1-yl)pyridin-3-yl]imidazo[1,2-a]pyrazine-6-carboxamide; N-(4-cyanophenyl)-3-(furan-2-yl)-N-methylimidazo[1,2-a]pyrazine-6-carboxamide; N-(4-cyanophenyl)-3-(furan-2-yl)-N-methylimidazo[1,2-a]pyrazine-6-carboxamide; 6-fluoro-1-({3-[2-(piperazin-1-yl)pyridin-4-yl]imidazo[1,2-a]pyrazin-6-yl}carbonyl)-1,2,3,4-tetrahydroquinoline; N-(4-chlorophenyl)-N-methyl-3-[4-(1,3,4-thiadiazol-2-yl)phenyl]imidazo[1,2-a]pyrazine-6-carboxamide; N-methyl-3-[4-(methylcarbamoyl)phenyl]-N-(5-methylpyridin-2-yl)imidazo[1,2-a]pyrazine-6-carboxamide; N-cyclohexyl-4-{6-[(6-fluoro-1,2,3,4-tetrahydroquinolin-1-yl)carbonyl]imidazo[1,2-a]pyrazin-3-yl}benzamide; 4-fluoro-N-methyl-N-[3-(1-methyl-1H-indazol-5-yl)imidazo[1,2-a]pyrazin-6-yl]benzamide; 1-[4-(5-{6-[(6-fluoro-1,2,3,4-tetrahydroquinolin-1-yl)carbonyl]imidazo[1,2-a]pyrazin-3-yl}pyridin-2-yl)piperazin-1-yl]ethan-1-one; 6-fluoro-1-({3-[3-(morpholin-4-ylmethyl)phenyl]imidazo[1,2-a]pyrazin-6-yl}carbonyl)-1,2,3,4-tetrahydroquinoline; N-(5-methylpyridin-2-yl)-3-[4-(trifluoromethyl)phenyl]imidazo[1,2-a]pyrazine-6-carboxamide; N-(4-ethylpyridin-2-yl)-N-methyl-3-[4-(trifluoromethyl)phenyl]imidazo[1,2-a]pyrazine-6-carboxamide; N-(4-cyanophenyl)-N-methyl-3-{4-[5-(methylamino)-1,3,4-thiadiazol-2-yl]phenyl}imidazo[1,2-a]pyrazine-6-carboxamide; 4-(6-{[(4-chlorophenyl)(methyl)amino]methyl}imidazo[1,2-a]pyrazin-3-yl)benzamide; N-(4-fluorophenyl)-N-methyl-3-{4-[5-(methylamino)-1,3,4-oxadiazol-2-yl]phenyl}imidazo[1,2-a]pyrazine-6-carboxamide; 4-[({3-[4-(difluoromethoxy)phenyl]imidazo[1,2-a]pyrazin-6-yl}methyl)(methyl)amino]benzonitrile; N-(4-chlorophenyl)-N-methyl-3-(1-methyl-1H-indazol-5-yl)imidazo[1,2-a]pyrazine-6-carboxamide; N-(4-cyanophenyl)-N-methyl-3-[4-(1H-pyrazol-5-yl)phenyl]imidazo[1,2-a]pyrazine-6-carboxamide; methyl N-(4-{6-[(4-cyanophenyl)(methyl)carbamoyl]imidazo[1,2-a]pyrazin-3-yl}phenyl)carbamate; N-(4-chlorophenyl)-N-methyl-3-{4-[5-(methylamino)-1,3,4-oxadiazol-2-yl]phenyl}imidazo[1,2-a]pyrazine-6-carboxamide; N-(4-cyanophenyl)-N-methyl-3-[4-(1H-pyrazol-3-yl)phenyl]imidazo[1,2-a]pyrazine-6-carboxamide; N-(4-cyanophenyl)-3-(4-acetamidophenyl)-N-methylimidazo[1,2-a]pyrazine-6-carboxamide; 3-(4-carbamoylphenyl)-N-(4-chlorophenyl)-N-methylimidazo[1,2-a]pyrazine-6-carboxamide; N-(4-cyanophenyl)-N-methyl-3-(1-methyl-1H-indazol-5-yl)imidazo[1,2-a]pyrazine-6-carboxamide; N-[3-(6-acetamidopyridin-3-yl)imidazo[1,2-a]pyrazin-6-yl]-4-fluoro-N-methylbenzamide; N-(6-chloropyridin-3-yl)-N-methyl-3-(1-methyl-1H-indazol-5-yl)imidazo[1,2-a]pyrazine-6-carboxamide; N-(4-fluoropyridin-2-yl)-N-methyl-3-[4-(trifluoromethyl)phenyl]imidazo[1,2-a]pyrazine-6-carboxamide; N-methyl-N-(5-methylpyridin-2-yl)-3-[4-(trifluoromethyl)phenyl]imidazo[1,2-a]pyrazine-6-carboxamide; N-(2-methoxypyridin-3-yl)-N-methyl-3-[4-(trifluoromethyl)phenyl]imidazo[1,2-a]pyrazine-6-carboxamide; N-(4-cyanophenyl)-N-methyl-3-[4-(1,3,4-thiadiazol-2-yl)phenyl]imidazo[1,2-a]pyrazine-6-carboxamide; (1,1-dioxido-2H-benzo[b][1,4]thiazin-4(3H)-yl)(3-(4-(trifluoromethyl)phenyl)imidazo[1,2-a]pyrazin-6-yl)methanone; 4-{6-[N-methyl(4-cyanobenzene)amido]imidazo[1,2-a]pyrazin-3-yl}benzamide; N-(2-chloro-1,3-thiazol-5-yl)-N-methyl-3-[4 (trifluoromethyl)phenyl]imidazo[1,2-a]pyrazine-6-carboxamide; 4-({3-[4-(trifluoromethyl)phenyl]imidazo[1,2-a]pyrazin-6-yl}carbonyl)-1,2,3,4-tetrahydroquinoxalin-2-one; N-methyl-N-{3-[4-(trifluoromethyl)phenyl]imidazo[1,2-a]pyrazin-6-yl}pyrimidine-5-carboxamide; N-methyl-N-(6-phenylpyridin-3-yl)-3-[4-(trifluoromethyl)phenyl]imidazo[1,2-a]pyrazine-6-carboxamide; N-methyl-N,3-bis[4-(trifluoromethyl)phenyl]imidazo[1,2-a]pyrazine-6-carboxamide; N-(4-methanesulfonylphenyl)-N-methyl-3-[4-(trifluoromethyl)phenyl]imidazo[1,2-a]pyrazine-6-carboxamide; N-(3-chloro-4-cyanophenyl)-N-methyl-3-[4-(trifluoromethyl)phenyl]imidazo[1,2-a]pyrazine-6-carboxamide; and N-(4-cyano-2-methoxyphenyl)-N-methyl-3-[4-(trifluoromethyl)phenyl]imidazo[1,2-a]pyrazine-6-carboxamide

### Embodiment 54

A compound, or a salt, tautomer or stereoisomer thereof, according to embodiment 1, wherein the compound is selected from the group consisting of: 3-(4-carbamoylphenyl)-N-(4-fluorophenyl)-N-methylimidazo[1,2-a]pyrazine-6-carboxamide; N-(4-cyanophenyl)-N-methyl-3-[4-(methylcarbamoyl)phenyl]imidazo[1,2-a]pyrazine-6-carboxamide; N-(4-cyanophenyl)-N-methyl-3-[4-(trifluoromethyl)phenyl]imidazo[1,2-a]pyrazine-6-carboxamide; 4-[methyl({3-[4-(trifluoromethyl)phenyl]imidazo[1,2-a]pyrazin-6-yl}methyl)amino]benzonitrile; 4-(6-{[(4-fluorophenyl)(methyl)amino]methyl}imidazo[1,2-a]pyrazin-3-yl)benzamide; 4-(6-{[(4-cyanophenyl)(methyl)amino]methyl}imidazo[1,2-a]pyrazin-3-yl)benzamide; N-(4-chlorophenyl)-N-methyl-3-[4-(methylcarbamoyl)phenyl]imidazo[1,2-a]pyrazine-6-carboxamide; N-(4-cyanophenyl)-3-(6-acetamidopyridin-3-yl)-N-methylimidazo[1,2-a]pyrazine-6-carboxamide; 4-(6-{[(4-chlorophenyl)(methyl)amino]methyl}imidazo[1,2-a]pyrazin-3-yl)benzamide; methyl N-(4-{6-[(4-cyanophenyl)(methyl)carbamoyl]imidazo[1,2-a]pyrazin-3-yl}phenyl)carbamate; 3-(4-carbamoylphenyl)-N-(4-chlorophenyl)-N-methylimidazo[1,2-a]pyrazine-6-carboxamide; and N-(4-cyanophenyl)-N-methyl-3-(1-methyl-1H-indazol-5-yl)imidazo[1,2-a]pyrazine-6-carboxamide.

### Embodiment 55

A pharmaceutical composition comprising at least one compound of any one of embodiments 1 to 54, or a pharmaceutically acceptable salt, or stereoisomer, thereof, and a pharmaceutically acceptable carrier, diluent or excipient.

### Embodiment 56

A pharmaceutical composition according to embodiment 55 further comprising a second agent.

### Embodiment 57

A pharmaceutical composition according to embodiment 56, wherein the second agent is an antimalarial drug selected from artemisinin, artemether, artesunate, arteflene, dihydroartemisinin, chlorproguanil, trimethoprim, chloroquine, quinine, mefloquine, amodiaquine, atovaquone, proguanil, lumefantrine, piperaquine, pyronaridine, halofantrine, pyrimethamine-sulfadoxine, quinacrine, pyrimethamine-dapsone, quinidine, amopyroquine, sulphonamides, primaquine, ferroquine, tafenoquine, arterolane, and pyronaridine.

### Embodiment 58

A compound according to any one of embodiments 1 to 54 or a pharmaceutical composition according to any one of embodiments 55 to 57 for use as a medicament.

### Embodiment 59

A method for treating, preventing, inhibiting, ameliorating, or eradicating the pathology and/or symptomology of a disease caused by a Plasmodium parasite, comprising administering to a subject a therapeutically effective amount of a compound according to any one of the embodiments 1 to 54 or a composition according to any one of the embodiments 55 to 57, wherein the administering may be in combination with a second agent.

### Embodiment 60

A method for treating, preventing, inhibiting, ameliorating, or eradicating the pathology and/or symptomology of a disease caused by a Plasmodium parasite, according to embodiment 59, wherein the disease is malaria.

### Embodiment 61

A method for treating, preventing, inhibiting, ameliorating, or eradicating the pathology and/or symptomology of a disease caused by a Plasmodium parasite according to any one of embodiments 59 to 60, wherein the Plasmodium parasite is at the blood stages.

### Embodiment 62

A method for treating, preventing, inhibiting, ameliorating, or eradicating the pathology and/or symptomology of a Plasmodium related disease caused by a Plasmodium parasite according to any one of embodiments 59 to 60, wherein the Plasmodium parasite is at the hepatic stages.

### Embodiment 63

A method for treating, preventing, inhibiting, ameliorating, or eradicating the pathology and/or symptomology of a Plasmodium related disease caused by a Plasmodium parasite according to any one of embodiments 59 to 62, wherein the Plasmodium parasite is selected from group consisting of Plasmodium falciparum, Plasmodium vivax, Plasmodium ovale, and Plasmodium malaria.

### Embodiment 64

A method for treating, preventing, inhibiting, ameliorating, or eradicating the pathology and/or symptomology of a Plasmodium related disease caused by a Plasmodium parasite according to any one of embodiments 59 to 62, wherein the Plasmodium parasite is Plasmodium falciparum.

### Embodiment 65

A method for treating, preventing, inhibiting, ameliorating, or eradicating the pathology and/or symptomology of a Plasmodium related disease caused by a Plasmodium parasite according to any one of embodiments 59 to 64, wherein the second agent is selected from a kinase inhibitor, an anti-malarial drug and an anti-inflammatory agent.

### Embodiment 66

A method for treating, preventing, inhibiting, ameliorating, or eradicating the pathology and/or symptomology of a disease caused by Plasmodium parasite according to embodiment 65, wherein the anti-malarial drug is selected from artemisinin, artemether, artesunate, arteflene, dihydroartemisinin, chlorproguanil, trimethoprim, chloroquine, quinine, mefloquine, amodiaquine, atovaquone, proguanil, lumefantrine, piperaquine, pyronaridine, halofantrine, pyrimethamine-sulfadoxine, quinacrine, pyrimethamine-dapsone, quinidine, amopyroquine, sulphonamides, primaquine, ferroquine, tafenoquine, arterolane, and pyronaridine.

### Embodiment 67

A method for treating, preventing, inhibiting, ameliorating, or eradicating the pathology and/or symptomology of a disease caused by Plasmodium parasite according to any one of embodiments 59 to 66, wherein the compound is administered prior to, simultaneously with, or after the second agent.

### Embodiment 68

A compound according to any one of embodiments 1 to 54 or a composition according to any one of embodiments 55 to 57 for treating, preventing, inhibiting, ameliorating, or eradicating the pathology and/or symptomology of a disease caused by a Plasmodium parasite.

### Embodiment 69

Use of a compound according to any one of embodiments 1-54 or a pharmaceutical composition according to embodiments 55 to 57 in the manufacture of a medicament for treating, preventing, inhibiting, or ameliorating the pathology and/or symptomology of a disease caused by a Plasmodium parasite, wherein said use may be in combination with a second agent.

As used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S.

Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

Depending on the choice of the starting materials and procedures, the compounds can be present in the form of one of the possible isomers or as mixtures thereof, for example as pure optical isomers, or as isomer mixtures, such as racemates and diastereoisomer mixtures, depending on the number of asymmetric carbon atoms. The present invention is meant to include all such possible isomers, including racemic mixtures, diasteriomeric mixtures and optically pure forms. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the terms “salt” or “salts” refers to an acid addition or base addition salt of a compound of the invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviate, inhibit, prevent and/or ameliorate a condition, or a disorder or a disease (i) mediated by Plasdmodium or (ii) associated with Plasdmodium activity, or (iii) characterized by activity (normal or abnormal) of Plasdmodium or (2) reduce or inhibit the activity of Plasdmodium; or (3) reduce or inhibit the growth of Plasdmodium. In another non-limiting embodiment, the term “a therapeutically effective amount” refers to the amount of the compound of the present invention that, when administered to a cell, or a tissue, or a non-cellular biological material, or a medium, is effective to at least partially reducing or inhibiting the activity of Plasdmodium; or at least partially reducing or inhibiting the growth of Plasdmodium.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The compounds of the present invention, including salts, hydrates and solvates thereof, may inherently or by design form polymorphs.

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Synthesis”, John Wiley and Sons, 1991.

Typically, the compounds of formula (I) can be prepared according to Schemes 1-5 provided infra., where variables Ring A, Ring B, Ring C, L, R₁, R₁₅, R₁₆, R₁₇, n, p and others are as defined in the Summary of the Invention. The following reaction schemes are given to be illustrative, not limiting, descriptions of the synthesis of compounds of the invention. Detailed descriptions of the synthesis of compounds of the Invention are given in the Examples, infra.

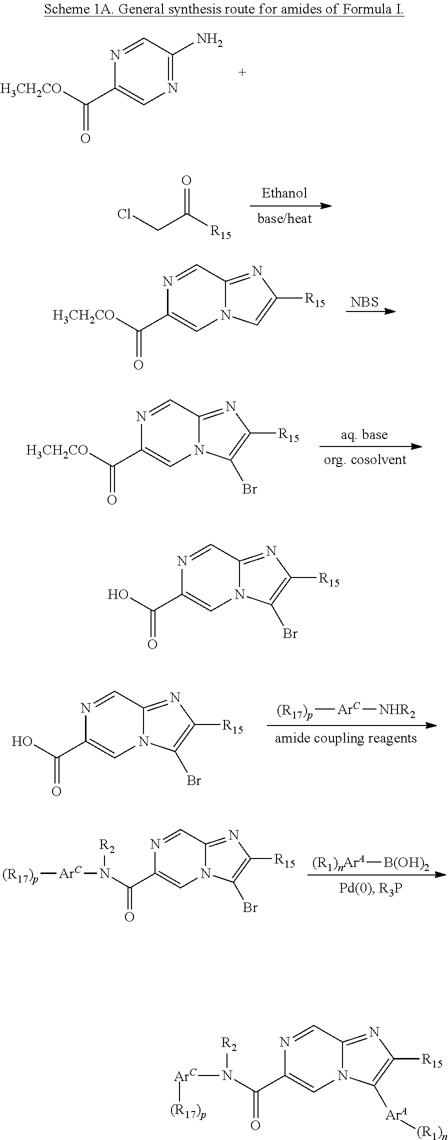

Ar^{A} and Ar^{C} represent rings A and C respectively in formula I

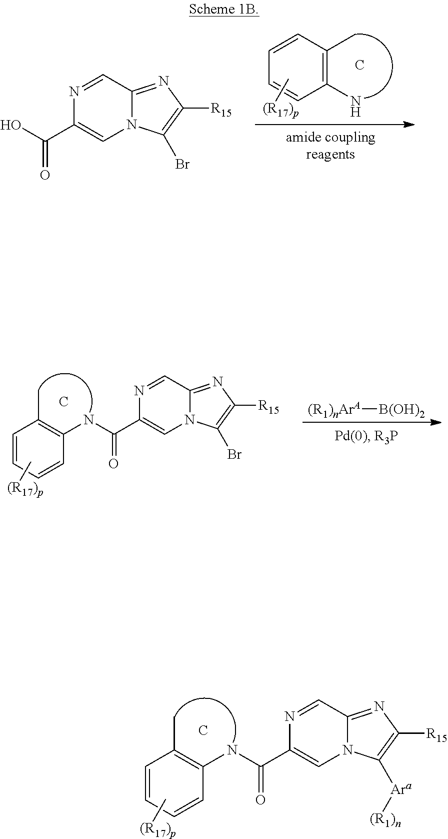

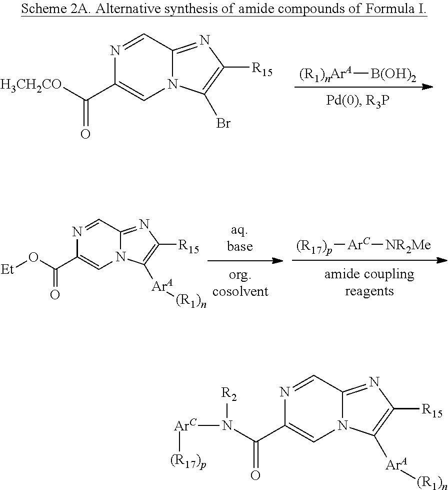

Ar^{A} and Ar^{C} represent rings A and C in formula I, respectively

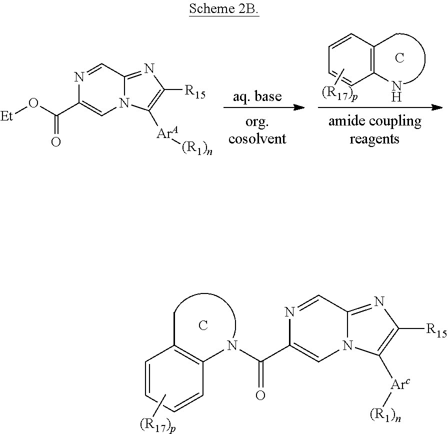

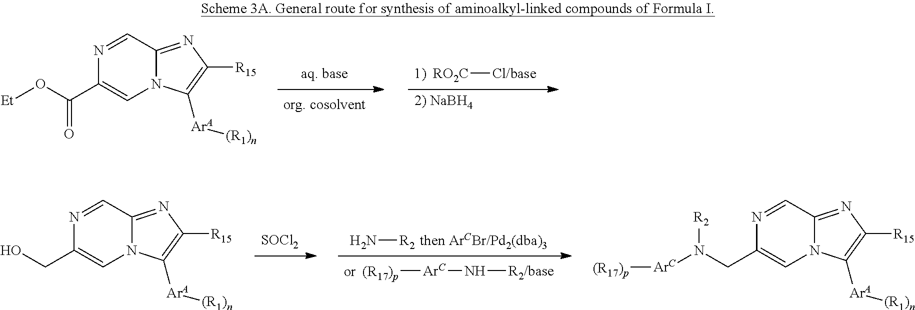

Ar^{A} and Ar^{C} represent rings A and C in Formula I, respectively.

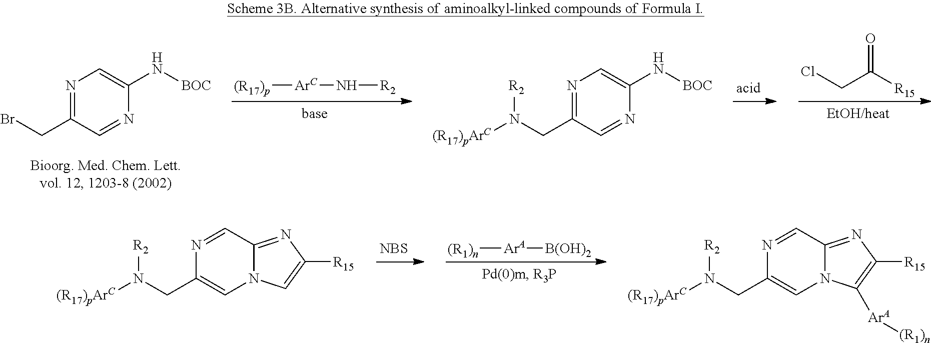

Ar^{A} and Ar^{C} represent rings A and C in Formula I, respectively.

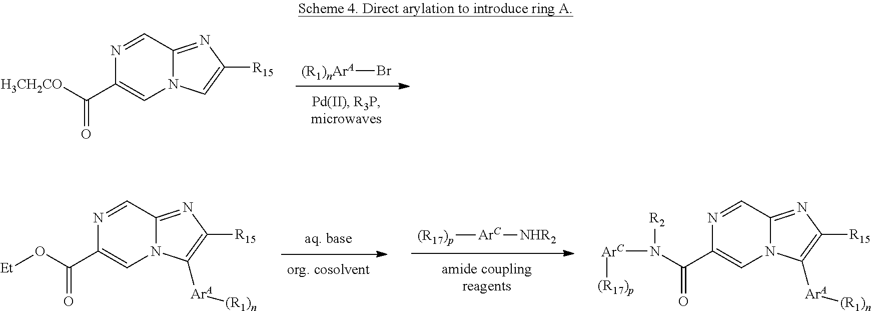

Ar^{A} and Ar^{C} represent rings A and C in Formula I, respectively.

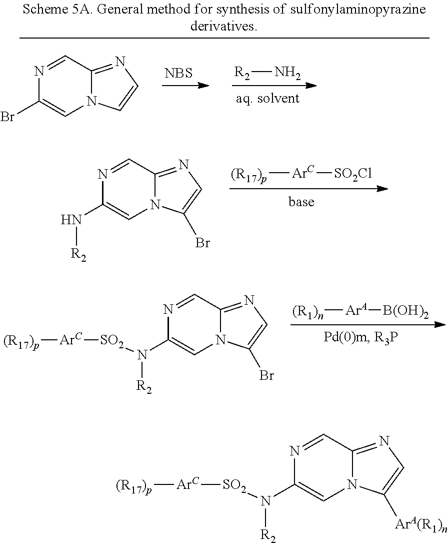

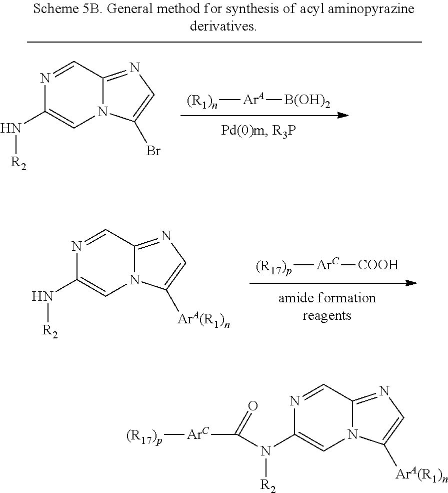

Ar^{A} and Ar^{C} represent rings A and C in Formula I, respectively.

### General Procedures for Boronic Ester Synthesis

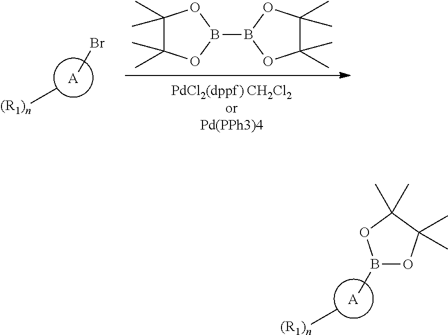

Boronic Ester Synthesis Procedure A: PdCl₂(dppf).CH₂Cl₂;

A mixture of bromo compound (1.0 eq.) and bis(pinacolato)diboron (1.1 eq.) and potassium acetate (2.0 eq.) dissolved in 1,4-dioxane (10 vol) was degassed with argon gas for 15 min. Subsequently, PdCl₂(dppf).CH₂Cl₂ (0.05 eq.) was added and the reaction mixture was stirred at 85-100° C. for 16 h. The reaction mixture (generally black color) was filtered and concentrated under reduced pressure. The resulting black mixture was used further without any purification.

### Boronic Ester Synthesis Procedure B: Pd(PPh₃)₄;

A mixture of bromo compound (1.0 eq.) and bis(pinacolato)diboron (1.1 eq.), and potassium acetate (2.0 eq.) dissolved in 1,4-dioxane (10 vol) was degassed with argon gas for 15 min. Subsequently, Pd₂(dba)₃ (0.05 eq.) and tricyclohexyl phosphine (0.05 eq.) were added and the reaction mixture was stirred at 90-110° C. for 16 h. The reaction mixture (generally black color) was filtered and concentrated under reduced pressure. The crude product was used further without any purification.

### General Procedures for Suzuki Couplings

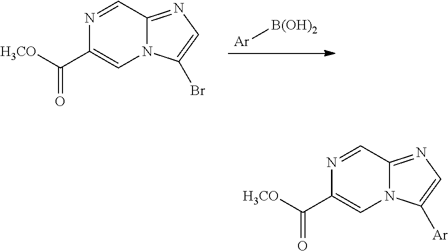

### Suzuki Procedure A: SiliaCat® DPP-Pd and K₂CO₃:

A mixture of bromo compound (0.2 mmol, 1.0 equiv.), boronic acid (0.22 mmol, 1.1 equiv.), and SiliaCat® DPP-Pd (0.25 mmol/g loading, 0.01 mmol, 0.05 equiv.) was treated with 660 μL dioxane and 220 μL 1 M aq. K₂CO₃ and the resulting mixture was allowed to heat overnight at 100° C. in a capped vial. The resulting black mixture was dry-loaded onto silica gel and was purified by silica gel chromatography, eluting with hexanes/EtOAc to give the desired product.

### Suzuki Procedure B: Pd(Dppf)Cl₂ and K₂CO₃ in the Microwave:

A mixture of aryl bromide (1.0 equiv.), aryl boronic acid (1.5 equiv.), K₂CO₃ (2.5 equiv.), and Pd(dppf)Cl₂ (0.05-0.15 equiv.) in THF/water was allowed to heat at 140° C. in a microwave reactor for 40 minutes. Purified by mass-triggered HPLC or silica gel chromatography to provide the desired product.

### Suzuki Procedure C: SiliaCat® DPP-Pd/Pd(dppf)Cl₂ and K₂HPO₄ in the Microwave:

A mixture of aryl bromide (1.0 equiv.), aryl boronic acid (1.5 equiv.), KH₂PO₄ (3.5 equiv.), and SiliaCat® DPP-Pd or Pd(dppf)Cl₂ (0.05-0.15 equiv.) in THF/water was allowed to heat at 150° C. in a microwave reactor for 40-60 minutes. Purified by mass-triggered HPLC or silica gel chromatography to provide the desired product.

### Suzuki Procedure D: Pd(dppf)Cl₂, K₂CO₃, DME-Water:

A mixture of aryl bromide (1.0 equiv.), aryl boronic acid (1.5 equiv.), K₂CO₃ (3.0 equiv.), and Pd(dppf)Cl₂ (0.05-0.15 equiv.) in DME/water was allowed to heat at 110° C. for two hours. Following extraction of the reaction mixture with CH₂Cl₂, the combined organic extracts were concentrated and the residue was purified by silica gel chromatography, eluting with hexanes/EtOAc to give the desired product.

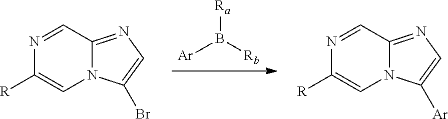

Suzuki Procedure E: Pd₂(dba)₃, P(O-Tolyl)₃, 2M KF Solution in Toluene/Ethanol:

A mixture of aryl bromide (1.0 equiv.), aryl boronic acid (1.2-2.0 equiv.), 2 M aq KF (3 equiv.), and Pd₂(dba)₃ (0.1 equiv.), P(o-tolyl)₃ (0.1 equiv.) in toluene:ethanol (7:3) was degassed and heated to 100° C. for 1-5 h. The crude products were purified by preparative TLC or silica gel chromatography to provide the desired product.

### Suzuki Procedure F: Pd(PPh₃)₄, 1N Na₂CO₃, Dioxane:

A mixture of aryl bromide (1.0 equiv.), aryl boronic acid (1.2-2.0 equiv.), 1 N Na₂CO₃ (2.0 equiv.), and Pd(PPh₃)₄ (0.2 equiv.) in 1,4-dioxane was degassed and heated in a sealed tube to 100° C. (microwave or conventional heating) for 2-6 h. The crude products were purified by preparative TLC or silica gel chromatography to provide the desired product.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure material.

Compounds of the invention and intermediates can also be converted into each other according to methods generally known to those skilled in the art.

Compounds of the invention are useful in the treatment and/or prevention of infections such as those caused by Plasmodium falciparum; Plasmodium vivax; Plasmodium ovale; and Plasmodium malaria, trypanosoma cruzi and parasites of the Leishmania genus, such as, for example, Leishmania donovani.

Plasmodia spp. which cause malaria belong to the phylum, Apicomplexa, which is a large and diverse group of protists that are human or animal parasites. These parasites are unicellular, spore-forming, and possess motile structures such as flagella or pseudopods at certain gamete stages. Most of these parasites possess a unique organelle called apicoplast and an apical comples structure involved in penetrating a host's cell. The pathogenesis associated the diseases caused by these parasites is due to repeated cycles of host-cell invasion, intracellular replication and host-cell lysis. Therefore, understanding parasite proliferation is essential for development of novel drugs and vaccines, for example, to treat malaria.

In vertebrate hosts, the parasite undergoes two main phases of development, the hepathocytic and erythrocytic phases, but it is the erythrocytic phase of its life cycle that causes severe pathology. During the erythrocytic phase, the parasite goes through a complex but well synchronized series of stages, suggesting the existence of tightly regulated signaling pathways.

Calcium serves as an intracellular messenger to control synchronization and development in the erythrocytic life phase. The Plasmodium spp. genomes reveal many sequence identities with calcium binding/sensing protein motifs that include Pf39, calmodulin, and calcium dependent protein kinases (CDPKs). Plasmodium CDPKs, Plasmodium CDPK3 and 4, have been shown to be involved in mosquito infection. CDPK4 has been demonstrated to be essential for the sexual reproduction in the midgut of mosquito by translating the calcium signal into a cellular response and regulating cell cycle progression in the male gametocyte. CDPK3 regulates ookinete gliding motility and penetration of the layer covering the midgut epithelium. P. falciparum CDPK1 (PfCDPK1) is expressed during late schizogony of blood stage and in the infectious sporozoite stage and is secreted to the parasitophorous vacuole by an acylation-dependent mechanism. It can be myristoylated and is abundantly found in detergent-resistant membrane fractions isolated from schizogony-phase parasites. Ontology based pattern identification analysis reveals that PfCDPK1 is clustered with genes associated with either parasite egress or erythrocyte invasion. Direct inhibition of PfCDPK1 can arrest the parasite erythrocytic life cycle progression in the late schizogony phase.

Therefore, kinase activity is distributed in all the stages of P. falciparum parasite maturation and kinase inhibitors of the present invention can be used for treating Plasmodium related diseases.

The in vitro cellular assay, infra, can be used to assess the activity of compounds of the invention against a variety of malarial parasite strains.

In accordance with the foregoing, the present invention further provides a method for preventing or treating malaria in a subject in need of such treatment, which method comprises administering to said subject a therapeutically effective amount of a compound selected from Formula I and Ia or a pharmaceutically acceptable salt thereof. The required dosage will vary depending on the mode of administration, the particular condition to be treated and the effect desired.

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Compounds of the invention can be administered in therapeutically effective amounts in combination with one or more therapeutic agents (pharmaceutical combinations). Non-limiting examples of compounds which can be used in combination with compounds of the invention are known anti-malarial drugs, for example, artemisinin, artemether, artesunate, arteflene, dihydroartemisinin, chlorproguanil, trimethoprim, chloroquine, quinine, mefloquine, amodiaquine, atovaquone, proguanil, lumefantrine, piperaquine, pyronaridine, halofantrine, pyrimethamine-sulfadoxine, quinacrine, pyrimethamine-dapsone, quinidine, amopyroquine, sulphonamides, primaquine, ferroquine, tafenoquine, arterolane, and pyronaridine, etc.

Where the compounds of the invention are administered in conjunction with other therapies, dosages of the co-administered compounds will of course vary depending on the type of co-drug employed, on the specific drug employed, on the condition being treated and so forth.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Biological Assays

The activity of a compound according to the present invention for inhibition of parasitemai in infected blood cells and liver cells can be assessed by the following assays. It is understood that the assays illustrate the invention without in any way limiting the scope of the invention.

Assay for P. falciparum Proliferation in Infected Human Blood Cells

Compounds of the invention can be assayed to measure their capacity to inhibit proliferation of P. falciparum parasitemia in infected red blood cells. This parasite proliferation assay measures the increase in parasite DNA content using a DNA intercalating dye, SYBR Green® (INVITROGEN®) which has a high affinity for double stranded DNA.

NF54 or 3D7 P. falciparum strain is grown in complete culturing media until parasitemia reaches 3% to 8% with O+ human erythrocytes. The selection of either strain is of convenience (3D7 is a clone of NF54) and does not make a difference to the assay. 20 μl of screening media is dispensed into 384 well assay plates. 50 nl of compounds of the invention (in DMSO), including antimalarial controls (mefloquine, pyrimethamine and artemisinin), are then transferred into the assay plates, as well as DMSO alone to serve as a negative control for inhibition. Then 30 μl of a suspension of a NF54 or 3D7 P. falciparum infected erythrocytes in screening media is dispensed into the assay plates such that the final hematocrit is 2.5% with a final parasitemia of 0.3%. The plates are placed in a 37° C. incubator for 72 hours in a low oxygen environment containing 93% N₂, 4% CO₂, and 3% O₂ gas mixture. 10 μl of lysis buffer (saponin, triton-X, EDTA) containing a 10× solution of SYBR Green I® in RPMI media is dispensed into the plates. The plates are lidded and kept at room temperature overnight for the lysis of the infected red blood cells. The fluorescence intensity is measured (excitation 425 nm, emission 530 nm) using the Envision™ system (Perkin Elmer). The percentage inhibition of 50%, EC₅₀, is calculated for each compound.

Using the P. falciparum Proliferation Assay above, compounds of the invention exhibit inhibitory efficacy (EC₅₀) of typically 10 μM or less, more typically less than 2 μM, most typically less than 200 nM. Compounds of the invention can significantly delay the increase in P. falciparum parasitemia. For example, 4-[({3-[4-(5-amino-1,3,4-thiadiazol-2-yl)phenyl]imidazo[1,2-a]pyrazin-6-yl}methyl)(methyl)amino]benzonitrile (Example 25); N,5-dimethyl-N-({3-[4-(1H-pyrazol-5-yl)phenyl]imidazo[1,2-a]pyrazin-6-yl}methyl)pyridin-2-amine (Example 47); N-(4-cyanophenyl)-N-[2-(dimethylamino)ethyl]-3-[4-(trifluoromethyl)phenyl]imidazo[1,2-a]pyrazine-6-carboxamide (Example 72); 4-(6-{[(4-fluorophenyl)(methyl)amino]methyl}imidazo[1,2-a]pyrazin-3-yl)benzamide (Example 222); 4-(6-{[(4-chlorophenyl)(methyl)amino]methyl}imidazo[1,2-a]pyrazin-3-yl)benzamide (Example 477), N-(4-fluorophenyl)-N-methyl-3-{4-[5-(methylamino)-1,3,4-oxadiazol-2-yl]phenyl}imidazo[1,2-a]pyrazine-6-carboxamide (Example 478); N-(4-chlorophenyl)-N-methyl-3-{4-[5-(methylamino)-1,3,4-oxadiazol-2-yl]phenyl}imidazo[1,2-a]pyrazine-6-carboxamide (Example 483), and 3-(4-carbamoylphenyl)-N-(4-chlorophenyl)-N-methylimidazo[1,2-a]pyrazine-6-carboxamide (Example 486), all have EC50 values of less than 50 nM.

The inhibitory efficacy of the compounds of the invention in delaying the increase in P. falciparum parasitemia in infected human blood cells is provided in Table 1.

TABLE 1
Inhibitory Efficacy of Compounds of the Invention in delaying
P. falciparum Proliferation in Infected Human Blood Cells

| Example No. | EC50 (nM) |
|---|---|
| 1 | 477 |
| 2 | 66 |
| 3 | 58 |
| 4 | 185 |
| 5 | 238 |
| 6 | 619 |
| 7 | >10,000 |
| 8 | 21 |
| 9 | 218 |
| 10 | 189 |
| 11 | 74 |
| 12 | 4850 |
| 13 | 41 |
| 14 | 9580 |
| 15 | 29 |
| 16 | 454 |
| 17 | 602 |
| 18 | 78 |
| 19 | 280 |
| 20 | >10,000 |
| 21 | 46 |
| 22 | 74 |
| 23 | 99 |
| 24 | 1120 |
| 25 | 6 |
| 26 | 8330 |
| 27 | >10,000 |
| 28 | 13 |
| 29 | 2036 |
| 30 | 2513 |
| 31 | 1205 |
| 32 | 135 |
| 33 | 4710 |
| 34 | 64 |
| 35 | 462 |
| 36 | 329 |
| 37 | 640 |
| 38 | 131 |
| 39 | 1081 |
| 40 | 3320 |
| 41 | 97 |
| 42 | 333 |
| 43 | >10,000 |
| 44 | 206 |
| 45 | 178 |
| 46 | >10,000 |
| 47 | 1241 |
| 48 | 5100 |
| 49 | 314 |
| 50 | 828 |
| 51 | >9720 |
| 52 | 187 |
| 53 | 12 |
| 54 | >9280 |
| 55 | 160 |
| 56 | 491 |
| 57 | 70 |
| 58 | 1141 |
| 59 | 35 |
| 60 | 2629 |
| 61 | 1371 |
| 62 | 82 |
| 63 | 387 |
| 64 | 116 |
| 65 | 354 |
| 66 | 5000 |
| 67 | 168 |
| 68 | >10,000 |
| 69 | 85 |
| 70 | 230 |
| 71 | 1305 |
| 72 | 439 |
| 73 | 624 |
| 74 | 24 |
| 75 | 37 |
| 76 | 35 |
| 77 | 116 |
| 78 | 181 |
| 79 | 214 |
| 80 | 258 |
| 81 | 382 |
| 82 | 2342 |
| 83 | 1572 |
| 84 | 1282 |
| 85 | 1100 |
| 86 | 2011 |
| 87 | 104 |
| 88 | 1736 |
| 89 | 2066 |
| 90 | 826 |
| 91 | 4300 |
| 92 | 349 |
| 93 | 4640 |
| 94 | >10,000 |
| 95 | 1050 |
| 96 | 9990 |
| 97 | 4510 |
| 98 | 1497 |
| 99 | 3880 |
| 100 | 297 |
| 101 | 33 |
| 102 | 53 |
| 103 | 51 |
| 104 | 57 |
| 105 | 41 |
| 106 | 24 |
| 107 | 218 |
| 108 | 110 |
| 109 | 120 |
| 110 | 188 |
| 111 | 128 |
| 112 | 103 |
| 113 | 81 |
| 114 | 76 |
| 115 | 82 |
| 116 | 230 |
| 117 | 92 |
| 118 | 135 |
| 119 | 507 |
| 120 | 147 |
| 121 | 259 |
| 122 | 266 |
| 123 | 387 |
| 124 | 217 |
| 125 | 369 |
| 126 | 125 |
| 127 | 236 |
| 128 | >10,000 |
| 129 | 800 |
| 130 | 176 |
| 131 | 140 |
| 132 | 206 |
| 133 | 338 |
| 134 | 490 |
| 135 | 81 |
| 136 | 320 |
| 137 | 226 |
| 138 | 849 |
| 139 | 1163 |
| 140 | 436 |
| 141 | 433 |
| 142 | 202 |
| 143 | 1565 |
| 144 | 366 |
| 145 | 648 |
| 146 | 462 |
| 147 | 564 |
| 148 | 1363 |
| 149 | 1517 |
| 150 | 3590 |
| 151 | 673 |
| 152 | 3170 |
| 153 | 2062 |
| 154 | 2689 |
| 155 | 2389 |
| 156 | 391 |
| 157 | 2009 |
| 158 | 2047 |
| 159 | 368 |
| 160 | 1747 |
| 161 | 1469 |
| 162 | 318 |
| 163 | 2634 |
| 164 | 961 |
| 165 | 4610 |
| 166 | 206 |
| 167 | 4870 |
| 168 | 558 |
| 169 | 497 |
| 170 | 4070 |
| 171 | 4010 |
| 172 | 1133 |
| 173 | 4250 |
| 174 | 4750 |
| 175 | 3280 |
| 176 | >10,000 |
| 177 | 6750 |
| 178 | 6510 |
| 179 | 4400 |
| 180 | 4340 |
| 181 | 723 |
| 182 | 2260 |
| 183 | 2402 |
| 184 | >10,000 |
| 185 | 7170 |
| 186 | 9170 |
| 187 | 1385 |
| 188 | >10,000 |
| 189 | 7890 |
| 190 | 6240 |
| 191 | 7730 |
| 192 | 7390 |
| 193 | 8590 |
| 194 | 6950 |
| 195 | >10,000 |
| 196 | 9350 |
| 197 | >10,000 |
| 198 | >10,000 |
| 199 | 7320 |
| 200 | >10,000 |
| 201 | 1083 |
| 202 | >10,000 |
| 203 | 6490 |
| 204 | 6510 |
| 205 | 7450 |
| 206 | >9760 |
| 207 | 4600 |
| 208 | 724 |
| 209 | 2702 |
| 210 | 1984 |
| 211 | 9390 |
| 212 | 137 |
| 213 | 41 |
| 214 | 31 |
| 215 | 86 |
| 216 | 5710 |
| 217 | 36 |
| 218 | 680 |
| 219 | 63 |
| 220 | 4040 |
| 221 | 4970 |
| 222 | 30 |
| 223 | 25 |
| 224 | 149 |
| 225 | 37 |
| 226 | 37 |
| 227 | 40 |
| 228 | 58 |
| 229 | 19 |
| 230 | 21 |
| 231 | 74 |
| 232 | 3990 |
| 233 | 138 |
| 234 | 148 |
| 235 | 483 |
| 236 | 127 |
| 237 | 100 |
| 238 | 152 |
| 239 | 6880 |
| 240 | 112 |
| 241 | 262 |
| 242 | 215 |
| 243 | 98 |
| 244 | 290 |
| 245 | 226 |
| 246 | 310 |
| 247 | 611 |
| 248 | 87 |
| 249 | 635 |
| 250 | 302 |
| 251 | 5520 |
| 252 | >8280 |
| 253 | 2464 |
| 254 | 67 |
| 255 | 155 |
| 256 | 240 |
| 257 | 92 |
| 258 | 76 |
| 259 | 414 |
| 260 | 399 |
| 261 | 408 |
| 262 | 97 |
| 263 | 436 |
| 264 | 277 |
| 265 | 318 |
| 266 | 234 |
| 267 | 424 |
| 268 | 112 |
| 269 | 651 |
| 270 | 605 |
| 271 | 207 |
| 272 | 295 |
| 273 | 186 |
| 274 | 224 |
| 275 | 468 |
| 276 | 310 |
| 277 | 1126 |
| 278 | 784 |
| 279 | 454 |
| 280 | 707 |
| 281 | 122 |
| 282 | 252 |
| 283 | 178 |
| 284 | 947 |
| 286 | 402 |
| 287 | 972 |
| 288 | 565 |
| 289 | 1503 |
| 290 | 784 |
| 291 | 1158 |
| 292 | 304 |
| 293 | 76 |
| 294 | 2125 |
| 295 | 237 |
| 296 | 1276 |
| 297 | 1291 |
| 298 | 1068 |
| 299 | 1128 |
| 300 | 2989 |
| 301 | 1198 |
| 302 | 1848 |
| 303 | 1280 |
| 304 | 452 |
| 305 | 457 |
| 306 | 607 |
| 307 | 1070 |
| 308 | 2024 |
| 309 | 611 |
| 310 | 2481 |
| 311 | 2782 |
| 312 | 2644 |
| 313 | 1322 |
| 314 | 1035 |
| 315 | 2418 |
| 316 | 1889 |
| 317 | 1900 |
| 318 | 2309 |
| 319 | 3141 |
| 320 | 2206 |
| 321 | 2263 |
| 322 | 2712 |
| 323 | 7640 |
| 324 | 1234 |
| 325 | 4140 |
| 326 | 2088 |
| 327 | 3310 |
| 328 | 1088 |
| 329 | 2756 |
| 330 | 1960 |
| 331 | 1742 |
| 332 | 3410 |
| 333 | 4130 |
| 334 | >5190 |
| 335 | 5340 |
| 336 | 6930 |
| 337 | >7160 |
| 338 | 4080 |
| 339 | 1255 |
| 340 | 6850 |
| 341 | 5790 |
| 342 | 393 |
| 343 | 6780 |
| 344 | 2988 |
| 345 | 2305 |
| 346 | 242 |
| 347 | 3260 |
| 348 | 4690 |
| 349 | 4680 |
| 350 | >5630 |
| 351 | 5620 |
| 352 | 876 |
| 353 | 3900 |
| 354 | 6070 |
| 355 | 2115 |
| 356 | 6680 |
| 357 | 5490 |
| 358 | 1172 |
| 359 | >10,000 |
| 360 | >4570 |
| 361 | 5150 |
| 362 | 6470 |
| 363 | 7020 |
| 364 | 8240 |
| 365 | 6140 |
| 366 | >6250 |
| 367 | >9280 |
| 368 | 7040 |
| 369 | 1161 |
| 370 | 8200 |
| 371 | >10,000 |
| 372 | >10,000 |
| 373 | >9880 |
| 374 | >9820 |
| 375 | >6830 |
| 376 | 9490 |
| 377 | 8190 |
| 378 | >7030 |
| 379 | 6260 |
| 380 | 4500 |
| 381 | 5110 |
| 382 | 8400 |
| 383 | >10,000 |
| 384 | 6620 |
| 385 | >10,000 |
| 386 | >10,000 |
| 387 | >10,000 |
| 388 | 762 |
| 389 | 1998 |
| 390 | 2646 |
| 391 | 2910 |
| 392 | 3250 |
| 393 | 3940 |
| 394 | 4910 |
| 395 | 5020 |
| 396 | 5720 |
| 397 | 6550 |
| 398 | 6840 |
| 399 | 6980 |
| 400 | 7850 |
| 401 | >10,000 |
| 402 | 3610 |
| 403 | >5980 |
| 404 | >7570 |
| 405 | >8820 |
| 406 | >8860 |
| 407 | >8970 |
| 408 | >9300 |
| 409 | >9340 |
| 410 | >10,000 |
| 411 | >7570 |
| 412 | 7880 |
| 413 | 7400 |
| 414 | >7910 |
| 415 | >10,000 |
| 416 | >10,000 |
| 417 | >10,000 |
| 418 | >10,000 |
| 419 | >8270 |
| 420 | >10,000 |
| 422 | >10,000 |
| 423 | >9660 |
| 424 | 291 |
| 425 | 1020 |
| 426 | 1951 |
| 427 | >9800 |
| 428 | 276 |
| 429 | 313 |
| 430 | 17 |
| 431 | 82 |
| 432 | 79 |
| 433 | 31 |
| 434 | 7090 |
| 435 | 214 |
| 436 | 9300 |
| 437 | 176 |
| 438 | >10,000 |
| 439 | 2761 |
| 440 | 140 |
| 441 | 6500 |
| 442 | 604 |
| 445 | >10,000 |
| 476 | 9 |
| 477 | 9 |
| 478 | 31 |
| 479 | 35 |
| 480 | 25 |
| 481 | 34 |
| 482 | 37 |
| 483 | 9 |
| 484 | 20 |
| 485 | 54 |
| 486 | 42 |
| 487 | 96 |
| 489 | 522 |
| 490 | 293 |
| 491 | 199 |
| 492 | 377 |
| 493 | 529 |
| 494 | 3350 |
| 495 | 405 |
| 496 | 2837 |
| 497 | >9320 |
| 498 | 5690 |
| 499 | 4510 |
| 500 | 353 |
| 501 | 1453 |
| 502 | 2004 |
| 503 | 60 |

### Assay for Proliferation of Parasite in Infected Liver Cells

Compounds of the invention can be assayed to measure their capacity to inhibit proliferation of parasites in liver cells. The proliferation is quantified by determine the number of infected cells by immunofluorescence.

### Parasites

Due to the difficulty of successfully infecting immortalized human liver cell lines with the human malaria sporozoites (liver-stage parasite), rodent malaria sporozoites from Plasmodium yoelii (17XNL) and P. berghei (ANKA) are the preferred surrogate. Sporozoites are obtained from Anopheles stephensi mosquitoes supplied by the New York University Insectary, which ships the malaria-infected mosquitoes 10-13 days following the ingestion of an infective blood meal.

### Cell Line

A transgenic HepG2 cell line expressing the tetraspanin CD81 receptor (HepG2-A16-CD81^{EGFP}) is used to increase the infectivity rate of rodent-malaria sporozoites into human cells. HepG2-A16-CD81^{EGFP} cells are stably transformed to express a GFP-CD81 fusion protein. A continuous in vitro culture of this line was maintained at 37° C. in 4% CO₂ in complete media (CM) which contains: DMEM (Invitrogen, Carlsbad, USA) supplemented with 10% FCS, 0.29 mg/ml glutamine, 100 units penicillin and 100 μg/ml streptomycin (SigmaAldrich, USA).

P. yoelii Sporozoite Invasion Assay

Twenty to twenty-six hours prior to sporozoite infection, 7.5×10³ HepG2-A16-CD81^{EGFP} cells are seeded into 384-well plates (Aurora 384 IQ-EB black plates with clear bottoms; 50 μl of 1.5×10⁵ cells/ml in CM). These plates are incubated at 37° C. with 4% CO₂ overnight. Two hours prior to infection, 50 nl of compound dissolved in DMSO (0.1% final DMSO concentration per well) were transferred with a PinTool (GNF Systems) into the assay plates (10 μM final concentration). A 1:3 serial dilution of atovaquone (10 μM at the highest final concentration) and wells treated only with DMSO were used as positive and negative controls, respectively.

Freshly dissected salivary glands from infected mosquitoes were homogenized in a glass tissue grinder, filtered twice through Nylon cell strainers (40 μm pore size, BD Falcon) and counted using a hemocytometer. The assay plate with HepG2-A16-CD81^{EGFP} cells and compound were then infected with 8×10³ sporozoites per well and the plates are subjected to a centrifugal force of 650×g to pellet the sporozoites onto the liver cell monolayer. The assay plate is incubated at 37° C. for 2 hours to permit sporozoite invasion, then the media is aspirated from the media plate, and replaced with 50 μl CM (containing a 5× concentration of penicillin/streptomycin; 500 units penicillin and 0.5 mg streptomycin per ml) per well. 50 nl of compound is re-introduced by PinTool and the assay plate incubated for 48 hours at 37° C. before quantification of infected cells by immunofluorescence. The increased antibiotic concentration does not interfere with the parasite or HepG2-A16-CD81^{EGFP} growth.

Atovaquone and uninfected wells were used as controls on each plate. Two replicate plates are tested for each assay.

### Immunofluorescence Quantification of Exo-Erythrocytic Forms (EEFs)

After fixing the cells by addition of 12.5 μl of 20% solution of paraformaldehyde (EMS, Hatfield, USA) to each assay well (4% final formaldehyde concentration), membranes were permeabilized with 0.5% Triton-X-100 (Thermo Fisher Scientific) and EEFs were stained using a mouse polyclonal serum raised against the Plasmodium yoelii heat shock protein 70 (PyHSP70), a DyLight 649 goat anti-mouse IgG, Fc(gamma) fragment specific secondary antibody (Jackson Immuno Research, Cat#115-495-071) and the Hoechst 33342 nucleic acid dye (Invitrogen, Carlsbad, USA). Stained EEFs were then quantified using the Opera Confocal High Content Screening System (PerkinElmer, Waltham, USA). Images were collected using a 20× objective lens (20×/0.45 NA, LWD Plan Fluor, Olympus) at a binning of 2, using a 365 nm Xeon arc lamp illumination to detect the Hoechst-labeled nuclei and 635 nm laser line to excite DyLight649-labeled parasites. The image resolution yielded was approximately 0.66 μm/pixel (˜0.43 μm 2/pixel). All images were analyzed using a custom Acapella™ (PerkinElmer) script parametrized for this assay. In brief, images from fields inside the well were first discarded as out-of focus when the intensity in the nuclear channel was too low. Then, HepG2-A16-CD81^{EGFP} cells were counted by detecting the nuclei labeled with Hoechst using the nuclei detection libraries available with Acapella™. Parasites were later segmented using the αPyHSP70 immuno-labeling signal, using a custom script library. Once the objects were segmented from the picture, morphological-based (e.g. size, roundness, etc) and intensity-based features were measured for each object detected in the image (i.e. nuclei and parasites). Infection ratio was set as the ratio between parasite number and number of nuclei counted in images considered as “in-focus”. EC₅₀ values were obtained using parasite area and a custom curve fitting model, and a standard logistic regression model was applied for curve fitting.

Using the P. yoelii Sporozoite Invasion Assay, compounds of the invention exhibit inhibitory efficacy (EC₅₀) of typically 1 μM or less, more typically 200 nM less. Selected compounds, for example, 4-[methyl({3-[4-(trifluoromethyl)phenyl]imidazo[1,2-a]pyrazin-6-yl}methyl)amino]benzonitrile (Example 213); 4-(6-{[(4-fluorophenyl)(methyl)amino]methyl}imidazo[1,2-a]pyrazin-3-yl)benzamide (Example 222), 4-(6-{[(4-cyanophenyl)(methyl)amino]methyl}imidazo[1,2-a]pyrazin-3-yl)benzamide (Example 223); N-(4-chlorophenyl)-N-methyl-3-[4-(methylcarbamoyl)phenyl]imidazo[1,2-a]pyrazine-6-carboxamide (Example 228); and 4-(6-{[(4-chlorophenyl)(methyl)amino]methyl}imidazo[1,2-a]pyrazin-3-yl)benzamide (Example 477) all have EC₅₀ values of less than 50 nM. Inhibitory efficacy of selected compounds in delaying the proliferation of P. yoelii Sporozoite in liver cells is listed in Table 2. Compounds of the invention hence show delay of the proliferation of P. yoelii in liver cells.

TABLE 2
Inhibitory Efficacy of Compounds of the Invention in delaying the
Proliferation of P. yoelii Sporozoite in Infected Liver Cells

| Example No. | EC50 (nM) |
|---|---|
| 11 | 56 |
| 38 | 290 |
| 76 | 263 |
| 213 | 14 |
| 222 | 7 |
| 223 | 11 |
| 228 | 32 |
| 236 | 189 |
| 477 | 11 |
| 482 | 64 |
| 486 | 120 |
| 487 | 2935 |
| 491 | 1108 |

### EXAMPLES

The present invention is further exemplified, but not to be limited, by the following examples and intermediates that illustrate the preparation of compounds of the invention. It is understood that if there appears to be a discrepancy between the name and structure of a particular compound, the structure is to be considered correct as the compound names were generated from the structures.

Temperatures are given in degrees Celsius. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

LC-MS Methods

### Method 1:

Waters Acquity Binary Gradient Pump; Waters Acquity PDA Detector. Waters Auto sampler; Waters Quattro micro API Mass Spectrometer with ESI and APCI ion source; UPLC Column: Waters Acquity; BEH; C18 1.7 um 50×2.1 mm; Mobile Phase: (A) H₂O+0.025% TFA and (B) Acetonitrile+0.025% TFA. Gradient: 0.4 mL/minute, initial 15% B ramp to 95% B over 3.0 minutes, then hold until 4.0 minutes, return to 15% B at 4.1 minutes until end of run, then equilibrated the column for 2.0 minutes; MS Scan: 100 to 1000amu in 0.5 seconds per channel; Diode Array Detector: 200 nm and 400 nm.

### Method 2:

Waters Acquity Binary Gradient Pump; Waters Acquity PDA Detector. Waters Auto sampler; Waters Quattro micro API Mass Spectrometer with ESI and APCI ion source; UPLC Column: Waters Acquity; BEH; C18 1.7 um 50×2.1 mm; Mobile Phase: (A) H₂O+0.025% TFA and (B) Acetonitrile+0.025% TFA. Gradient: 0.4 mL/minute, initial 20% B ramp to 90% B over 2.0 minutes, then hold until 4.0 minutes, return to 20% B at 4.1 minutes until end of run, then equilibrated the column for 2.0 minutes; MS Scan: 100 to 1000amu in 0.5 seconds per channel; Diode Array Detector: 200 nm and 400 nm.

### Method 3:

Waters Acquity Binary Gradient Pump; Waters Acquity PDA Detector. Waters Auto sampler; Waters Acquity Evaporative Light Scattering Detector; Waters Quattro micro API Mass Spectrometer with ESI and APCI ion source; UPLC Column: Waters Acquity; BEH; C18 1.7 um 100×2.1 mm; Mobile Phase: (A) H₂O+0.025% TFA and (B) Acetonitrile+0.025% TFA. Gradient: 0.3 mL/minute, initial 10% B ramp to 80% B over 4.0 minutes, then hold until 6.0 minutes, return to 10% B at 6.1 minutes until end of run, then equilibrated the column for 2.5 minutes; MS Scan: 100 to 1000amu in 0.5 seconds per channel; Diode Array Detector: 200 nm and 400 nm; Drift tube temperature: 50° C. and N2 gas flow: 40Psi for ELSD Detector.

### Method 4:

Agilent 1200 sl/6140 system; UPLC Column: Waters Acquity; HSS T3; C18 1.8 um 50×2.0 mm; Mobile Phase: (A) H₂O+0.05% TFA and (B) Acetonitrile+0.035% TFA. Gradient: 0.9 mL/minute, initial 10% B ramp to 100% B over 1.95 minutes, then return to 10% B at 2.00 minutes until end of run, MS Scan: 100 to 1000amu in 0.5 seconds per channel; Diode Array Detector: 190 nm and 400 nm; Drift tube temperature: 50° C. and N2 gas flow: 40Psi for ELSD Detector.

### Method 5

Agilent 1100 sl/1946 system; UPLC Column: Waters atlantis; C18 1.8 um 50×2.0 mm; Mobile Phase: (A) H₂O+0.05% TFA and (B) Acetonitrile+0.035% TFA. Gradient: 1.0 mL/minute, initial 10% B ramp to 90% B over 3.00 minutes, then return to 10% B at 3.5 minutes until end of run, MS Scan: 100 to 1000amu in 0.5 seconds per channel; Diode Array Detector: 190 nm and 400 nm; Drift tube temperature: 50° C. and N2 gas flow: 40Psi for ELSD Detector.

Analytical method: WATERS ZQ SHIMADZU LEAP CTC, ZORBAX SB-C8 30*4.6 mm, 3.5 um, UV1:220 nm, UV2:254 nm, A:H₂O (0.03% TFA), B:CH₃CN (0.05% TFA), Flow: 2.000 (ml/min), Time/% B: 0/5, 1.90/95, 2.30/95, 2.31/5, 2.50/5

### Synthesis of Intermediates

### Synthesis of Ethyl pyrrolo[1,2-a]pyrazine-3-carboxylate (I-1)

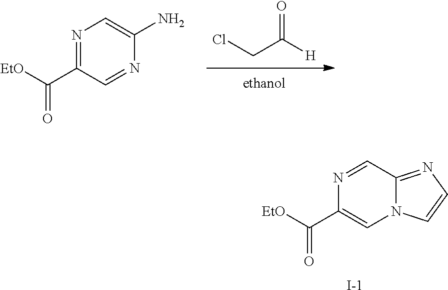

Chloroacetaldehyde (9.5 mL of a 50 wt % solution in water) was added to a mixture of aminopyrazine (2.00 g, 12.0 mmol), sodium bicarbonate (2.00 g, 23.8 mmol) and absolute ethanol (100 mL). The reaction was heated to 90° C. for 16 hours. After cooling to room temperature, the reaction was concentrated and purified by flash chromatography (silica, 0-10% methanol/chloroform) to give pure I-1 as a tan colored solid. ¹H NMR (400 MHz, CDCl3) δ 9.18 (s, 1H), 9.01 (d, J=1.4, 1H), 7.93 (d, J=1.1, 1H), 7.84 (s, 1H), 4.51 (q, J=7.1, 3H), 1.46 (t, J=7.1, 4H).

### Synthesis of Ethyl 2-methylimidazo[1,2-a]pyrazine-6-carboxylate (I-2)

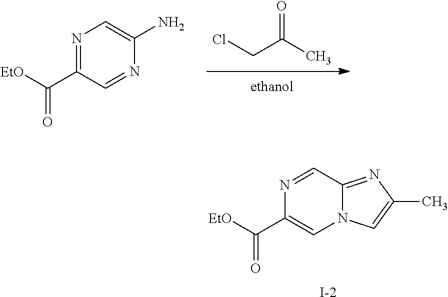

A mixture of aminopyrazine, 1-chloropropan-2-one and ethanol was heated in a microwave synthesizer at 180° C. for 60 minutes. After cooling to room temperature, the solvent is removed and the dark brown residue is purified by flash chromatography (silica, 30-60% ethyl acetate/hexanes) to give I-2 as a tan colored solid. ¹H NMR (400 MHz, CDCl3) δ 9.00 (s, 1H), 8.89 (d, J=1.3, 1H), 7.53 (d, J=23.3, 1H), 4.48 (q, J=7.1, 2H), 2.53 (s, 3H), 1.44 (t, J=7.1, 3H).

### Synthesis of Ethyl 2-(trifluoromethyl)imidazo[1,2-a]pyrazine-6-carboxylate (I-3)

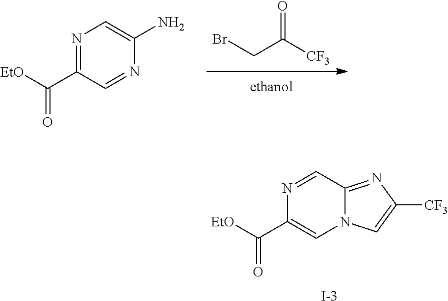

A mixture of aminopyrazine, 3-bromo-1,1,1-trifluoropropan-2-one and ethanol was heated in a microwave synthesizer at 180° C. for 60 minutes. After cooling to room temperature, the solvent is removed and the dark brown residue is purified by flash chromatography (silica, 30-60% ethyl acetate/hexanes) to give I-3 as a tan colored solid. ¹H NMR (400 MHz, CDCl3) δ 9.24 (s, 1H), 8.99 (d, J=1.4, 1H), 8.10 (s, 1H), 4.51 (q, J=7.1, 2H), 1.46 (t, J=7.1, 3H).

### Synthesis of Ethyl 3-bromoimidazo[1,2-a]pyrazine-6-carboxylate (I-4)

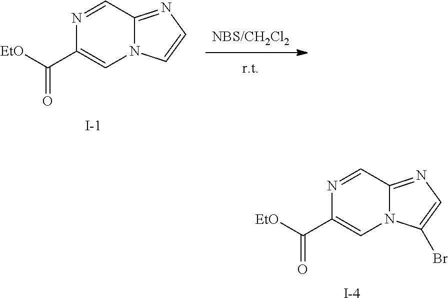

NBS (139 mg, 0.785 mmol) was added in one portion to a solution of ethyl pyrrolo[1,2-a]pyrazine-3-carboxylate (100 mg, 0.523 mmol) and dichloromethane (2.0 mL) at room temperature. After three hours, the reaction was filtered and the filtrate was purified by flash chromatography (silica, 10-100% ethyl acetate/hexanes) to give pure I-4 as a white colored solid. ¹H NMR (400 MHz, DMSO) δ 9.15 (s, 1H), 8.83 (s, 1H), 8.10 (s, 1H), 4.40 (q, J=7.2 2H), 1.37 (t, J=7.2, 3H).

### Synthesis of Ethyl 3-(4-(trifluoromethyl)phenyl)imidazo[1,2-a]pyrazine-6-carboxylate and methyl 3-(4-(trifluoromethyl)phenyl)imidazo[1,2-a]pyrazine-6-carboxylate (I-5 & I-6)

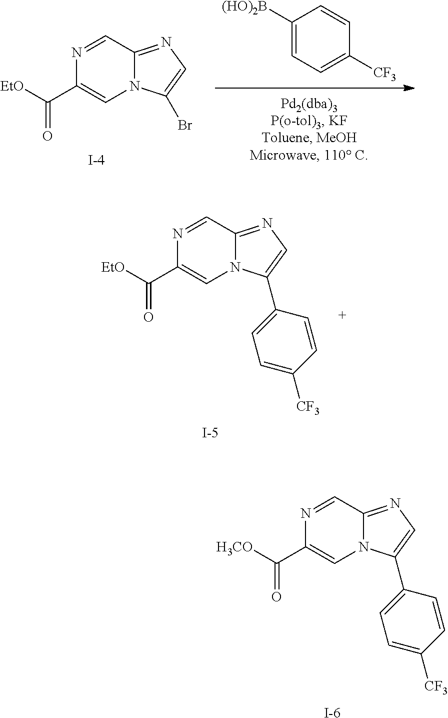

Pd₂(dba)₃ (58 mg, 0.0629 mmol) was added to a degassed mixture of 3-bromoimidazo[1,2-a]pyrazine-6-carboxylate (170 mg, 0.629 mmol), 4-trifluoromethylphenyl boronic acid (237 mg, 1.25 mmol), P(o-tol)₃ (23 mg, 0.075 mmol), KF (0.70 mL of a 2.0 M solution in water), toluene (8.4 mL), and methanol (5.6 mL). The reaction was heated in the microwave at 110° C. for 20 minutes. The reaction was cooled to room temperature, filtered and concentrated. The crude mixture was purified by flash chromatography (silica, 0-10% methanol/chloroform) to give a mixture of I-5 and I-6 as a white colored solid.

### Synthesis of 3-(4-(trifluoromethyl)phenyl)imidazo[1,2-a]pyrazine-6-carboxylic acid (I-7)

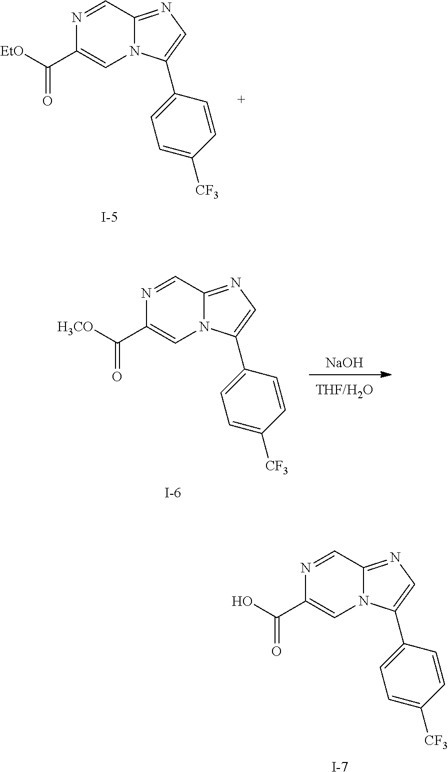

NaOH (1.25 mL of a 1.0 N solution in water) was added to a solution of ethyl 3-(4-(trifluoromethyl)phenyl)imidazo[1,2-a]pyrazine-6-carboxylate and methyl 3-(4-(trifluoromethyl)phenyl)imidazo[1,2-a]pyrazine-6-carboxylate (200 mg, 0.629 mmol) and tetrahydrofuran (1.25 mL). The reaction stirred at room temperature for three hours. The solvent was removed in vacuo. The crude reaction mixture was taken up in DI water and acidified to pH 3, resulting in a white precipitate that was collected by vacuum filtration to give I-7 as a white colored solid. ¹H NMR (400 MHz, DMSO) δ 9.13 (s, 1H), 9.01 (s, 1H), 8.23 (s, 1H), 8.05-7.95 (m, 4H).

### Synthesis of N-(4-cyanophenyl)-3-(4-(trifluoromethyl)phenyl)imidazo[1,2-a]pyrazine-6-carboxamide (I-8)

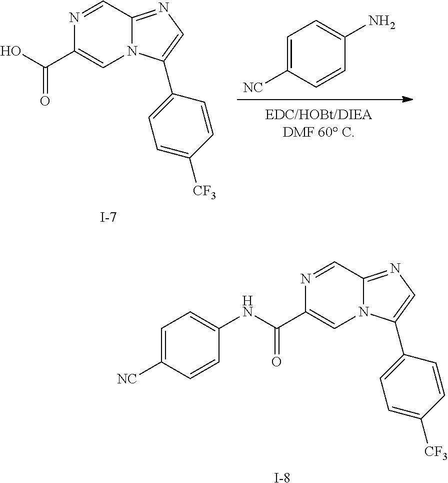

EDC (19 mg, 0.098 mmol) was added to a solution of 3-(4-(trifluoromethyl)phenyl)imidazo[1,2-a]pyrazine-6-carboxylic acid (20 mg, 0.0653 mmol), 4-cyanoaniline (18 mg, 0.098 mmol), diisopropylethylamine (0.045 mL, 0.261 mmol), HOBt (14 mg, 0.098 mmol) and DMF (0.75 mL). The reaction stirred at room temperature for 6 hours and was then heated to 60° C. for 10 hours. After cooling to room temperature, the crude reaction mixture was diluted with DI water (10 mL). The aqueous solution was extracted with ethyl acetate (3×3.0 mL). The organic extracts were combined and washed with DI water (2×2.0 mL) and brine (2×2.0 mL). The organic solution was dried over MgSO₄ and purified by flash chromatography (silica, 20-100% ethyl acetate/hexanes) to give pure I-8 as a white colored solid.

### Synthesis of Ethyl 3-(4-(trifluoromethyl)phenyl)imidazo[1,2-a]pyrazine-6-carboxylate (I-5)

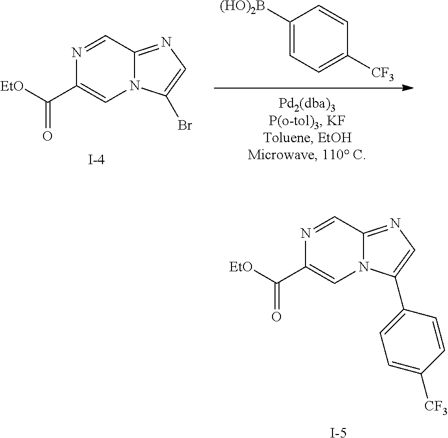

A solution of 4-trifluoromethylbenzene boronic acid (446 mg, 2.35 mmol) in degassed ethanol (4.2 mL) was added to a mixture of ethyl 3-bromoimidazo[1,2-a]pyrazine-6-carboxylate (200 mg, 1.18 mmol), P(o-tol)₃ (44 mg, 0.142 mmol) and degassed toluene (6.3 mL). Pd₂(dba)₃ (108 mg, 0.118 mmol) was added followed by 1.20 mL of an aqueous 2N KF solution. The reaction was heated in the microwave for 12 minutes at 110° C. The solvent was removed and the crude reaction mixture was purified by flash chromatography (silica, 25-100% ethyl acetate/hexanes) to give pure I-5 as a tan colored solid.

Alternatively, it can also be synthesized using following protocol.

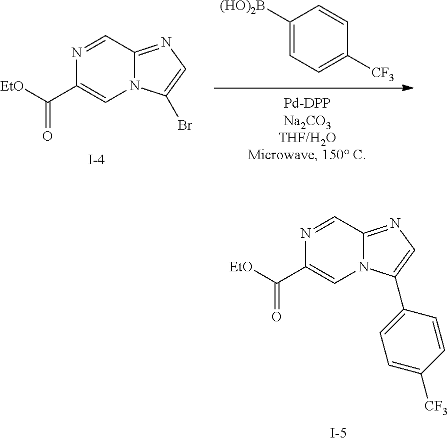

Pd-DPP (Palladium diphenylphosphine supported on silica, silicycle) (200 mg) was added to a mixture of 3-bromoimidazo[1,2-a]pyrazine-6-carboxylate (500 mg, 1.86 mmol), 4-trifluoromethylbenzene boronic acid (349 mg, 1.86 mmol), Na₂CO₃ (623 mg, 7.42 mmol), tetrahydrofuran (12 mL) and DI water (3.0 mL). The reaction was heated in a microwave reactor at 150° C. for 1 hour. After cooling to room temperature, the solvent was removed in vacuo. The crude material was purified by flash chromatography to give I-5 as a white solid (silica, 25-100% ethyl acetate/hexanes).

### Synthesis of N-methyl-3-(4-(trifluoromethyl)phenyl)imidazo[1,2-a]pyrazine-6-carboxamide (I-9)

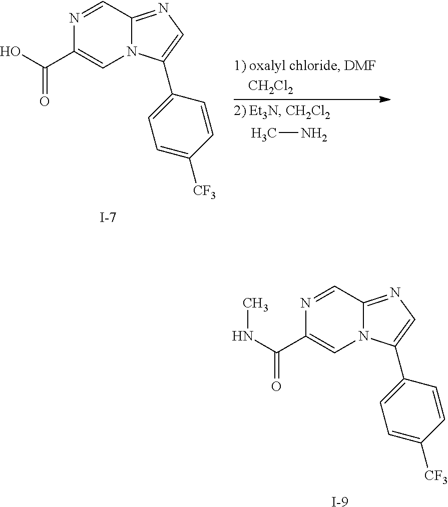

Oxalyl chloride (0.050 mL, 0.59 mmol) was added dropwise to a solution of 3-(4-(trifluoromethyl)phenyl)imidazo[1,2-a]pyrazine-6-carboxylic acid (50 mg, 0.16 mmol), DMF (2 drops) and dichloromethane (2.0 mL). After 30 minutes at room temperature, the solvent was removed. The reaction remained on the high vacuum pump for 60 minutes. Fresh dichloromethane (2.0 mL) was added, followed by a solution of N-methylamine (0.30 mL, 0.587 mmol). Triethylamine (0.15 mL, 1.1 mmol) was added and the reaction stirred at room temperature for 1 hour. The reaction was purified by flash chromatography to give I-9 as a white solid (silica, 50-100% ethyl acetate/hexanes).

### Synthesis of 3-bromoimidazo[1,2-a]pyrazine-6-carboxylic acid (I-10)

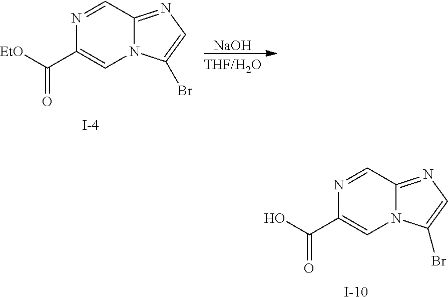

NaOH (4.0 mL of a 1.0N aqueous solution) was added to a mixture of ethyl 3-bromoimidazo[1,2-a]pyrazine-6-carboxylate (650 mg, 2.42 mmol) and tetrahydrofuran (4.0 mL). The reaction mixture was heated to 60° C. for 90 minutes. After cooling to room temperature, the solvent was removed in vacuo. The crude product was taken up in DI water and acidified to pH 3 with 1.0N HCl resulting in the formation of 1-10 as a white precipitate that was collected by vacuum filtration. ¹H NMR (400 MHz, DMSO) δ 9.15 (d, J=1.4, 1H), 8.83 (d, J=1.4, 1H), 8.13 (s, 1H).

### Synthesis of 3-bromo-N-(4-chlorophenyl)-N-methylimidazo[1,2-a]pyrazine-6-carboxamide (I-11)

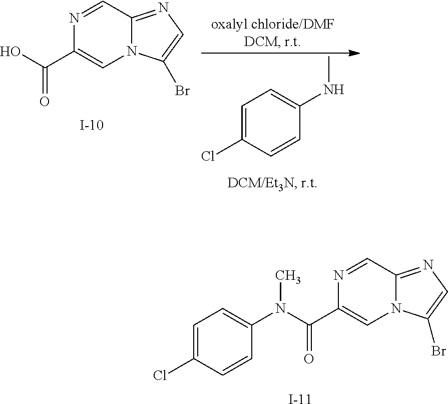

Oxalyl chloride (0.083 mL of a 2.0M solution in dichloromethane) was added drop wise to a solution of 3-bromoimidazo[1,2-a]pyrazine-6-carboxylic acid (200 mg, 0.833 mmol), DMF (2 drops) and dry dichloromethane (5.0 mL) at room temperature (gas evolution). After 30 minutes at room temperature, the solvent was removed in vacuo. The crude acid chloride was dissolved in dry dichloromethane (5.0 mL) and a solution of 4-(methylamino)chlorobenzene (235 mg, 1.670 mmol in 2.0 mL of dichloromethane) was added drop wise at room temperature. Triethylamine (0.25 mL, 2.44 mmol) was added and the reaction stirred at room temperature for 3 hours. The solvent was removed in vacuo and the crude material was purified by flash chromatography to give I-11 as a tan solid (silica, 10-100% ethyl acetate/hexanes). ¹H NMR (400 MHz, CDCl3) δ 8.62 (d, J=1.3, 1H), 8.59 (s, 1H), 7.78 (s, 1H), 7.21 (d, J=8.6, 2H), 7.04 (d, J=7.7, 2H), 3.51 (s, 3H).

### Synthesis of tert-butyl(5-(((4-cyanophenyl)(methyl)amino)methyl)pyrazin-2-yl)carbamate (I-12)

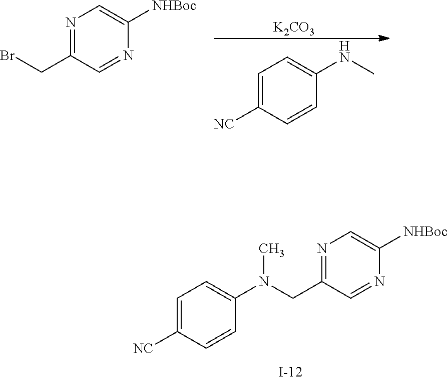

4-(N-methylamino)benzonitrile (574 mg, 4.35 mmol) was added to a mixture of tert-butyl(5-(bromomethyl)pyrazin-2-yl)carbamate (Prepared following the methods outlined in: Bioorganic & Medicinal Chemistry Letters, 2002, 12, 1203-1208.) (1.0 g, 3.48 mmol), potassium carbonate (4.8 g, 34.8 mmol) and acetone (40.0 mL). The reaction was heated to reflux for two hours. LCMS analysis showed 80% conversion. Stir for an additional hour. The reaction was diluted with acetone (40.0 mL) and filtered to remove the solids. The filtrate was concentrated and purified by flash chromatography to give I-12 as a white solid (silica 10-100% ethyl acetate/hexanes).

### Synthesis of 4-(((5-aminopyrazin-2-yl)methyl)(methyl)amino)benzonitrile (I-13)

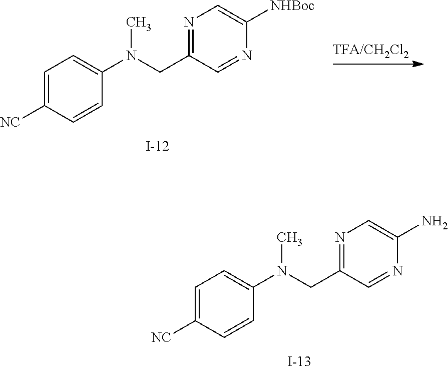

TFA (2.0 mL) was added to a solution of 1-12 (250 mg, 0.74 mmol) and dichloromethane (7.0 mL). The reaction stirred at room temperature for 12 hours. The solvent was removed and fresh dichloromethane (5 mL) was added. The organic layer was washed with NaHCO₃ (2×3 mL), dried over MgSO₄ and concentrated to give I-13 as a colorless oil.

### Synthesis of 4-((imidazo[1,2-a]pyrazin-6-ylmethyl)(methyl)amino)benzonitrile (I-14)

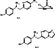

Chloroacetaldehyde was added to a solution of aminopyrazine and ethanol. The reaction was heated to reflux for 6 hours. After cooling to room temperature, the solvent was removed. The crude mixture was taken up in dichloromethane and washed with saturated sodium bicarbonate. The organic layer was dried over MgSO₄, concentrated and purified by flash chromatography to give I-14 as a white solid (silica, 0-10% methanol/dichloromethane). ¹H NMR confirms the structure. ¹H NMR (400 MHz, CDCl3) δ 9.08 (s, 1H), 7.78 (s, 2H), 7.60 (s, 1H), 7.47 (d, J=9.1, 2H), 6.72 (d, J=9.1, 2H), 4.72 (s, 2H), 3.21 (s, 3H).

### Synthesis of 4-(((3-bromoimidazo[1,2-a]pyrazin-6-yl)methyl)(methyl)amino)benzonitrile (1-15)

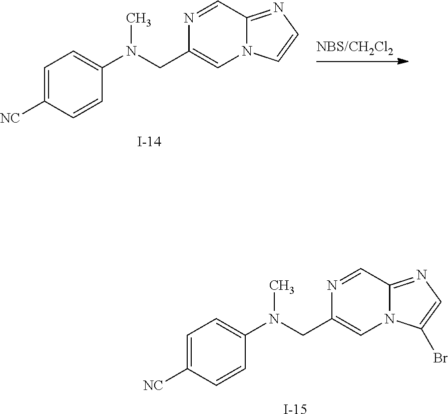

NBS was added to a solution of imidazolopyrazine and dichloromethane at −78° C. The reaction was allowed to slowly warm to room temperature. LCMS indicated that the reaction was complete. Mono-bromo and di-bromo and the starting material were present. The solvent was removed and the crude material was purified by flash chromatography to give I-15 as a tan solid (silica, 50-100% ethyl acetate/hexanes).

### Synthesis of (3-(4-(trifluoromethyl)phenyl)imidazo[1,2-a]pyrazin-6-yl)methanol (I-16)

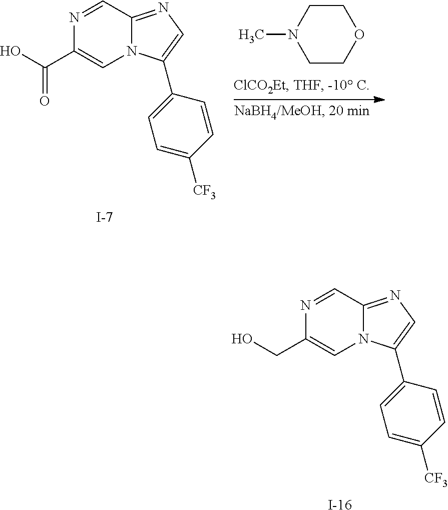

A mixture of imidazolopyridazine carboxylic acid (50 mg, 0.200 mmol) and THF (1.0 mL) was cooled to −10° C. (ethylene glycol/CO₂). N-methyl morpholine (0.022 mL, 0.200 mmol) and ethyl chloroformate (0.019 mL, 0.200 mmol) were added. The reaction stirred at −10° C. for 10 minutes. Sodium borohydride (23 mg, 0.600 mmol) was added and the reaction was allowed to warm to 0° C. (ice/water). Methanol (2.0 mL) was then added dropwise over 10 minutes. The solvent was removed. The crude material was taken up in ethyl acetate and washed with 1N HCl and 5% aqueous sodium bicarbonate, dried over MgSO₄ and purified by flash chromatography to give I-16 (silica, 0-25% MeOH/CH₂Cl₂).

### Synthesis of N-methyl-1-(3-(4-(trifluoromethyl)phenyl)imidazo[1,2-a]pyrazin-6-yl)methanamine (I-17)

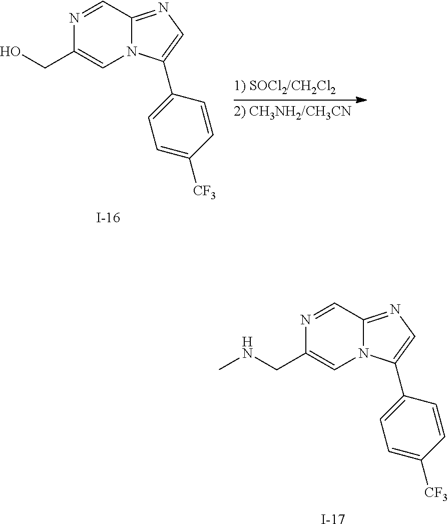

Thionyl chloride (0.018 mL, 0.256 mmol) was added to a solution of alcohol (50 mg, 0.171 mmol) and dichloromethane (2.0 mL) at 0° C. After 10 minutes at 0° C., the reaction was allowed to warm to room temperature and stir for 1 hour. The solvent was removed to give the 6-chloromethylimidazolopyrazine, which was carried on without further purification. Methylamine (0.15 mL of a 40% solution in water) was added to a solution of the 6-chloromethylimidazolopyrazine (0.171 mmol) and acetonitrile (2 mL) at 0° C. The reaction was warmed to 50° C. for 2 hours. The solvent was removed and the product was purified by flash chromatography (silica, 10% 7N NH₃ in methanol/dichloromethane) to give I-17 as a white foam.

### Synthesis of 6-bromoimidazo[1,2-a]pyrazine (I-18)

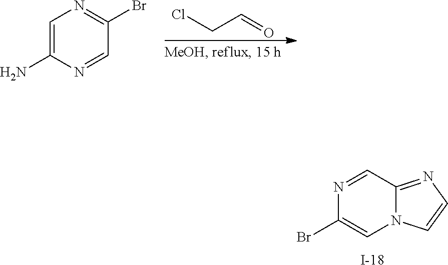

To a solution of 5-bromopyrazin-2-amine (11.3 g, 0.065 mol, 1.0 equiv) in CH₃OH (150 mL) was added 2-chloroacetaldehyde (40% in water, 65 g, 0.33 mol, 5.1 equiv). The resulting solution was refluxed for 15 hrs then the resulting mixture was concentrated under vacuum. The residue was applied onto a silica gel column with ethyl acetate/petroleum ether (20:1). This resulted in 12 g of 6-bromoimidazo[1,2-a]pyrazine as a brown solid. (ES, m/z): [M+H⁺] 197.

### Synthesis of 3,6-dibromoimidazo[1,2-a]pyrazine (I-19)

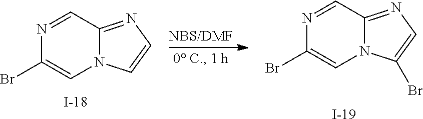

To a solution of 6-bromoimidazo[1,2-a]pyrazine (1.43 g, 7.26 mmol, 1.0 equiv) in DMF (50 mL) was added NBS (1.39 g, 7.81 mmol, 1.1 equiv) at −5˜0° C. The resulting solution was stirred at 0° C. for 1 hr. The resulting mixture was diluted with ethyl acetate (300 mL) then washed with brine. The organic layer was dried and concentrated to obtain 1.8 g of the crude product as a white solid. (ES, m/z): [M+H⁺] 278; H-NMR (300 MHz, CDCl₃): δ 8.92 (d, J=1.2 Hz, 1H), 8.28 (s, 1H), 7.84 (d, J=1.2 Hz, 1H).

### Synthesis of 3-bromo-N-methylimidazo[1,2-a]pyrazin-6-amine (I-20)

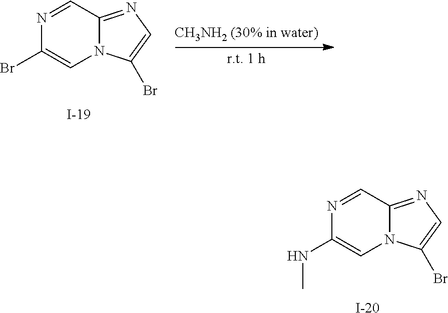

A solution of 3,6-dibromoimidazo[1,2-a]pyrazine (1.8 g, 6.49 mmol, 1.0 equiv) in CH₃NH₂ (30% in water, 50 mL) was stirred at r.t. for 1 hr then concentrated. The residue was applied onto a silica gel column with ethyl acetate/petroleum ether (1:1). This resulted in 1.08 g of 3-bromo-N-methylimid-azo[1,2-a]pyrazin-6-amine as a white solid. (ES, m/z): [M+H⁺] 227; H-NMR (300 MHz, CDCl₃): δ 8.70 (d, J=1.5 Hz, 1H), 7.64 (s, 1H), 7.02 (d, J=1.2 Hz, 1H), 4.41 (brs, 1H), 2.94 (d, J=5.4 Hz, 3H)

### Synthesis of N-methyl-3-(4-(trifluoromethyl)phenyl)imidazo[1,2-a]pyrazin-6-amine (I-21)

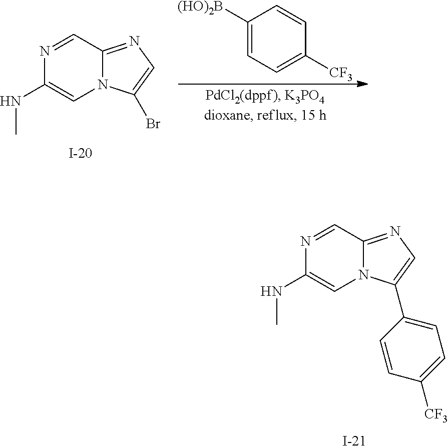

To a solution of 3-bromo-N-methylimidazo[1,2-a]pyrazin-6-amine (1.08 g, 4.76 mmol, 1.0 equiv) in dioxane (50 mL) was added 4-(trifluoromethyl)phenylboronic acid (1.80 g, 9.52 mmol, 2 equiv), PdCl₂(dppf) (0.347 g, 0.476 mmol) and K₃PO₄ (3.026 g, 14.3 mmol, 3 equiv) under nitrogen atmosphere. The resulting solution was refluxed for 15 hr. The resulting solution was filtered. The filtrate was concentrated then applied onto a silica gel column with CH₂Cl₂/CH₃OH (100:1). This resulted in 1.01 g of N-methyl-3-(4-(trifluoromethyl)phenyl)imidazo[1,2-a]pyrazin-6-amine as solid. (ES, m/z): [M+H]⁺ 293; H-NMR (300 MHz, CDCl₃): δ 8.50 (d, J=1.5 Hz, 1H), 7.87-7.95 (m, 5H), 7.47 (d, J=1.5 Hz, 1H), 2.84 (s, 3H).

### Synthesis of 2-(4-bromobenzylidene)hydrazinecarboxamide (I-22)

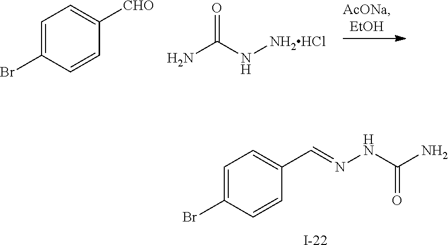

4-bromobenzaldehyde (3.7 gm, 20.0 mmol, 1.0 eq.), semicarbazide (2.23 gm, 20.0 mmol, 1.0 eq.) and sodium acetate (3.28 gm, 40.0 mmol, 2.0 eq.) were mixed in ethanol (50 ml) and stirred for 3 hours. The product was formed as a solid. The reaction was filtered and washed with ether to yield desired product (3.2 gm, 62%). The product was characterized by reverse phase HPLC using method 4. (ES, m/z): [M+H⁺] 242.1. The product was carried to the next step without purification.

### Synthesis of 5-(4-bromophenyl)-1,3,4-oxadiazol-2-amine (I-23)

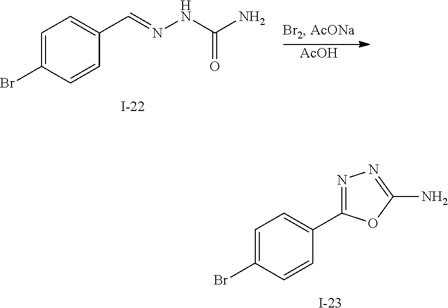

Semicarbazone (2.58 gm, 0.0 mmol, 1.0 eq.) and sodium acetate sodium acetate (3.28 gm, 40.0 mmol, 2.0 eq.) were dissolved in 30-40 ml of glacial acetic acid with continuous stirring. Bromine (0.7 ml in 5 ml of glacial acetic acid) was added slowly to it. Solution was stirred for 1 hour and then poured on crushed ice. The resulting solid was separated, dried to yield desired product (650 mg, 25%). ¹H NMR (400 MHz, MeOD) δ 7.86-7.77 (m, 1H), 7.74-7.63 (m, 1H).

### Synthesis of 5-(4-(4,4,5,5-tetramethyl-1,3,2-dioxaborolan-2-yl)phenyl)-1,3,4-oxadiazol-2-amine (I-24)

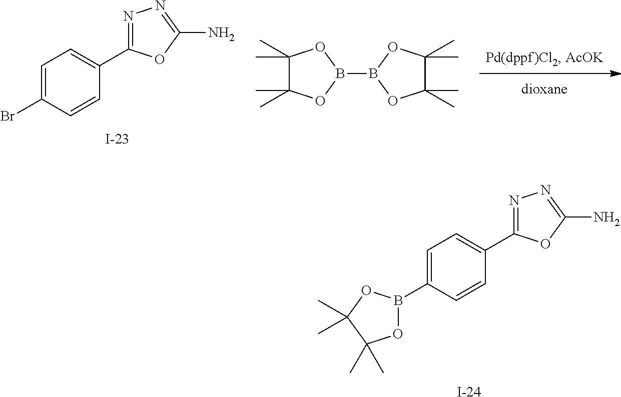

In a 40 ml vial, oxadiazole derivative (600 mg, 2.51 mmol, 1.0 eq.), bispinacolotodiboron (637.5 mg, 2.51 mmol, 1.0 eq.), Pd(dppf)Cl₂ (204.9 mg, 0.251 mmol, 0.1 eq.) and potassium acetate (1.47 gm, 15.06 mmol, 6.0 eq.) were mixed in dioxane (15 ml) and heated to 80° C. for 8 hours. LCMS indicated that the product was formed in major amount. The crude reaction was purified in the presence of silica (10-15 gm) and purified using hexanes:ethyl acetate (0-100%) to yield desired boron ester (275 mg, 38%). The product was characterized by reverse phase HPLC using method 4. (ES, m/z): [M+H⁺] 297.9. Retention time=1.61 mins.

### Synthesis of 2-(4-(1,1-difluoroethyl)phenyl)-4,4,5,5-tetramethyl-1,3,2-dioxaborolane (I-25)

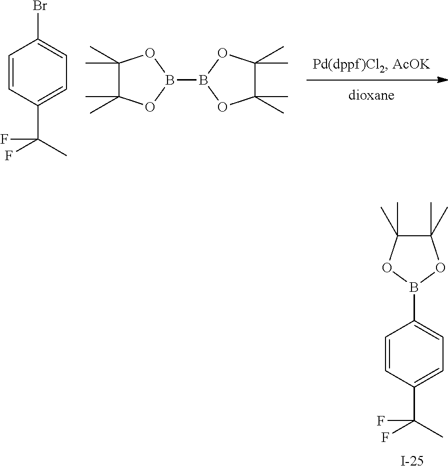

In a 40 ml vial, bromo derivative (250 mg, 1.13 mmol, 1.0 eq.), bispinacolotodiboron (288.6 mg, 1.13 mmol, 1.0 eq.), Pd(dppf)Cl₂ (92.7 mg, 0.114 mmol, 0.1 eq.) and potassium acetate (354.5 mg, 3.41 mmol, 3.0 eq.) were mixed in dioxane (10 ml) and heated to 80° C. for 8 hours. LCMS indicated that the product was formed in major amount. The crude reaction was purified in the presence of silica (10-15 gm) and purified using hexanes:ethyl acetate (0-100%) to yield desired boron ester. The product was characterized by reverse phase HPLC using method 4. (ES, m/z): [M+H⁺] 269.0. Retention time=1.60 mins.

### Synthesis of 6-(4,4,5,5-tetramethyl-1,3,2-dioxaborolan-2-yl)isoquinolin-1(2H)-one (I-26)

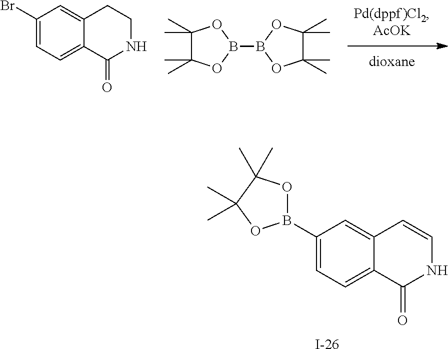

In a 40 ml vial, bromo derivative (250 mg, 1.11 mmol, 1.0 eq.), bispinacolotodiboron (288.6 mg, 1.11 mmol, 1.0 eq.), Pd(dppf)Cl₂ (90.7 mg, 0.111 mmol, 0.1 eq.) and potassium acetate (327.5 mg, 3.331 mmol, 3.0 eq.) were mixed in dioxane (10 ml) and heated to 80° C. for 48 hours. LCMS indicated that the product was formed in major amount. The crude reaction was purified in the presence of silica (10-15 gm) and purified using hexanes:ethyl acetate (0-100%) to yield desired boron ester. The product was characterized by reverse phase HPLC using method 4. (ES, m/z): [M+H⁺] 271.9. Retention time=1.19 mins.

### Synthesis of 1-methyl-5-(4-(4,4,5,5-tetramethyl-1,3,2-dioxaborolan-2-yl)phenyl)-1H-pyrazole (I-27)

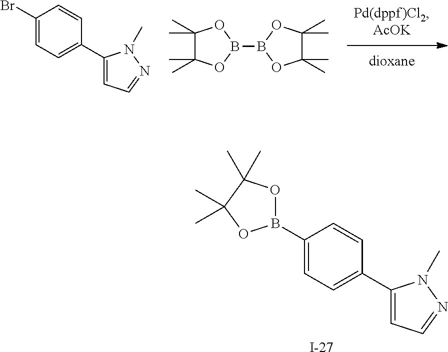

In a 40 ml vial, bromo derivative (236 mg, 1.06 mmol, 1.0 eq.), bispinacolotodiboron (253.6 mg, 1.06 mmol, 1.0 eq.), Pd(dppf)Cl₂ (86.4 mg, 0.106 mmol, 0.1 eq.) and potassium acetate (321.8 mg, 3.17 mmol, 3.0 eq.) were mixed in dioxane (10 ml) and heated to 80° C. for 48 hours. LCMS indicated that the product was formed in major amount. The crude reaction was purified in the presence of silica (10-15 gm) and purified using hexanes:ethyl acetate (0-100%) to yield desired boron ester. The product was characterized by reverse phase HPLC using method 4. (ES, m/z): [M+H⁺] 284.9. Retention time=1.42 mins.

### Synthesis of N-methyl-[1,2,4]triazolo[4,3-a]pyridin-5-amine (I-28)

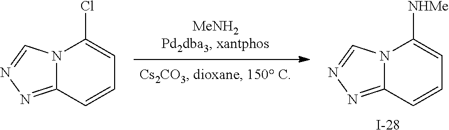

To a solution of chloro derivative (300 mg, 1.96 mmol, 1.0 eq.) in 10 mL of dioxane were added, Pd₂(dba)₃ (90 mg, 0.098 mmol, 0.05 eq.), Xantphos (113 mg, 0.196 mmol, 0.1 eq.) and Cs₂CO₃ (1274 mg, 3.92 eq., 2.0 eq.) at room temperature. The reaction mixture was degassed for 5-10 mins and then methyl amine (121.6 mg, 3.92 eq., 2.0 eq.) was added and stirred at 100° C. under N₂ for 8 hours. HPLC/MS test showed that the starting material (I) was consumed and the desired product was one of the major peaks ([M+1]). The reaction mixture was cooled to room temperature and solid was filtered off. The resulting filtrate was concentrated and the product was purified by column chromatography using dichloromethane/methanol to yield the desired product (50 mg, 17%). The product was characterized by reverse phase HPLC using method 4. (ES, m/z): [M+H⁺] 149.0. Retention time=0.36 mins.

### Synthesis of 3-methyl-5-(4,4,5,5-tetramethyl-1,3,2-dioxaborolan-2-yl)-1H-indazole (I-29)

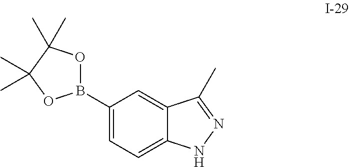 [SMILES: Cc1n[nH]c2ccc(B3OC(C)(C)C(C)(C)O3)cc12]

The boronic ester was synthesized from the corresponding bromide using a protocol as described in the synthesis of I-24. The product was characterized by reverse phase HPLC using method 4. (ES, m/z): [M+H⁺] 258.9. Retention time=1.28 mins.

### Synthesis of 4-((2-methoxyethyl)amino)benzonitrile (I-30)

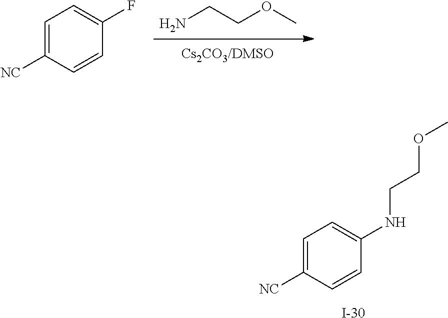

2-methoxyethanamine (300 mg, 4.00 mmol) was added to a mixture of 4-fluorobenzonitrile (250 mg, 2.07 mmol), cesium carbonate (750 mg, 2.30 mmol) and DMSO (4.0 mL). The reaction was heated to 60° C. for 4 hours. After cooling to room temperature, the reaction was diluted with water. The aqueous solution was extracted with ethyl acetate (3×15 mL). The combined extracts were dried over MgSO₄ and concentrated. The crude residue was purified by flash chromatography (silica, 0-10% methanol/dichloromethane) to give 4-((2-methoxyethyl)amino)benzonitrile (I-30) as a white solid.

### Synthesis of 4-((2-(tetrahydro-2H-pyran-4-yl)ethyl)amino)benzonitrile (I-31)

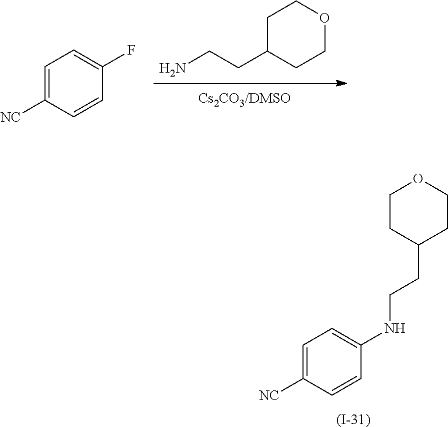

The above intermediate was prepared following the procedure outlined for the synthesis of 4-((2-methoxyethyl)amino)benzonitrile (I-30).

### Synthesis of 4-((2-(dimethylamino)ethyl)amino)benzonitrile (I-32)

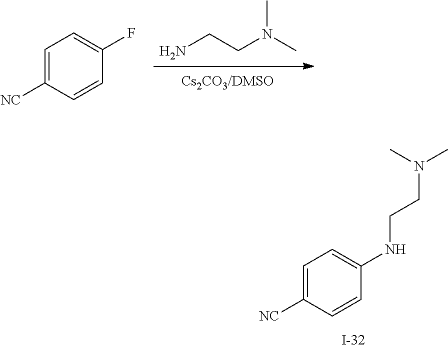

The above intermediate was prepared following the procedure outlined for the synthesis of 4-((2-methoxyethyl)amino)benzonitrile. ¹H NMR (400 MHz, DMSO) δ 7.44 (d, J=8.8, 2H), 6.66 (d, J=8.9, 2H), 3.15 (q, J=6.6, 3H), 2.42 (t, J=6.6, 2H), 2.18 (s, 6H).

### Synthesis of tert-butyl(2-((4-cyanophenyl)amino)ethyl)carbamate (I-33)

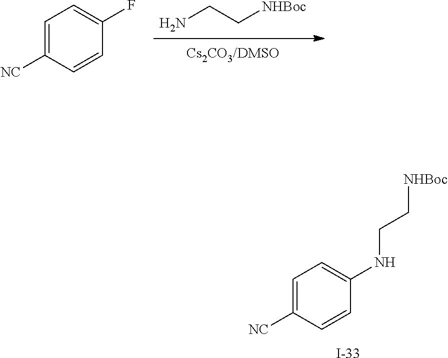

The above intermediate was prepared following the procedure outlined for the synthesis of 4-((2-methoxyethyl)amino)benzonitrile. ¹H NMR (400 MHz, DMSO) δ 7.45 (d, J=8.8, 2H), 6.64 (d, J=8.9, 2H), 3.18-2.95 (m, 4H), 1.38 (s, 9H).

### Preparation of N-methylanilines

### Synthesis of N-methyl-4-(1-methyl-1H-pyrazol-4-yl)aniline (I-34)

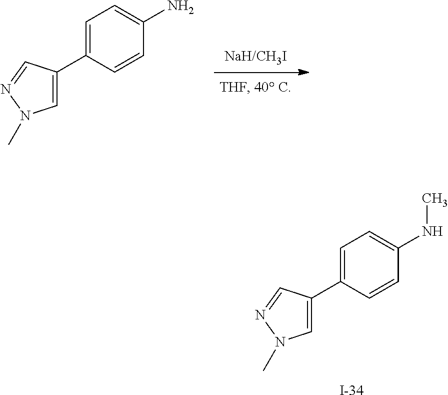

A solution of 4-(1-methyl-1H-pyrazol-4-yl)aniline (254 mg g, 1.47 mmol) in THF (5 mL) was added to a suspension of NaH (60% dispersion in mineral oil, 60 mg, 1.47 mmol) and THF (150 mL). The reaction stirred at room temperature for 3 hours. Iodomethane (0.090 mL, 1.47 mmol) was added and the reaction was heated to 40° C. for 9 hours. After cooling to room temperature, the solvent was removed. The crude material was tritrated in dichloromethane (100 mL) and filtered to remove any salts. The filtrate was purified by flash chromatography (silica, 10-80% ethyl acetate/hexanes). ¹H NMR (400 MHz, DMSO) δ 7.88 (s, 1H), 7.65 (d, J=0.8, 1H), 7.27 (d, J=8.6, 2H), 6.52 (d, J=8.6, 2H), 5.59 (d, J=5.1, 1H), 3.82 (s, 3H), 2.67 (d, J=5.1, 3H).

### Preparation of N-methylaminopyridines

### Synthesis of 6-chloro-N-methylpyridin-3-amine (I-35)

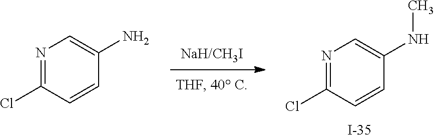

A solution of 3-amino-6-chloropyridine (2.46 g, 19.2 mmol) in THF (50 mL) was added to a suspension of NaH (60% dispersion in mineral oil, 768 mg, 19.2 mmol) and THF (150 mL). The reaction stirred at room temperature for 3 hours. Iodomethane (1.2 mL, 19.2 mmol) was added and the reaction was heated to 40° C. for 9 hours. After cooling to room temperature, the solvent was removed. The crude material was tritrated in dichloromethane (100 mL) and filtered to remove any salts. The filtrate was purified by flash chromatography (silica, 10-80% ethyl acetate/hexanes). ¹H NMR (400 MHz, CDCl3) δ 7.72 (d, J=3.1, 1H), 7.06 (d, J=8.6, 1H), 6.83 (dd, J=3.1, 8.6, 1H), 3.78 (s, 1H), 2.84 (d, J=19.7, 3H).

### Synthesis of 5-chloro-N-methylpyridin-2-amine (I-36)

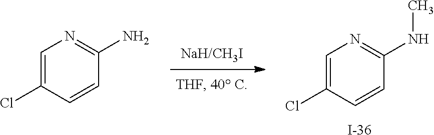

The above intermediate was prepared following the procedure outlined for the synthesis of 6-chloro-N-methylpyridin-3-amine. ¹H NMR (400 MHz, CDCl3) δ 8.00 (d, J=2.5, 1H), 7.35 (dd, J=2.6, 8.9, 1H), 6.31 (d, J=8.9, 1H), 4.58 (s, 1H), 2.87 (d, J=5.2, 3H).

### Synthesis of 6-(methylamino)nicotinonitrile (I-37)

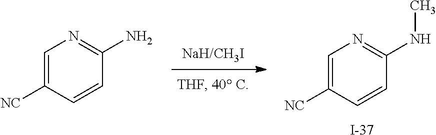

The above intermediate was prepared following the procedure outlined for the synthesis of 6-chloro-N-methylpyridin-3-amine. ¹H NMR (400 MHz, CDCl3) δ 8.35 (d, J=2.0, 1H), 7.57 (dd, J=2.0, 8.8, 1H), 6.36 (dd, J=0.5, 8.9, 1H), 5.11 (s, 1H), 2.96 (d, J=5.2, 3H).

### Synthesis of 5-fluoro-N-methylpyridin-2-amine (I-38)

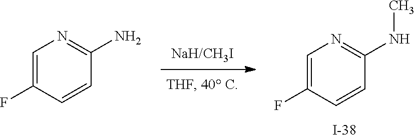

The above intermediate was prepared following the procedure outlined for the synthesis of 6-chloro-N-methylpyridin-3-amine. ¹H NMR (400 MHz, CDCl3) δ 7.93 (d, J=3.0, 1H), 7.19 (ddd, J=3.0, 7.9, 9.0, 1H), 6.31 (dd, J=3.4, 8.9, 1H), 4.48 (s, 1H), 2.86 (s, 3H).

### Synthesis of 6-fluoro-N-methylpyridin-2-amine (I-39)

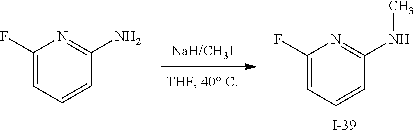

The above intermediate was prepared following the procedure outlined for the synthesis of 6-chloro-N-methylpyridin-3-amine. ¹H NMR (400 MHz, CDCl3) δ 7.46 (q, J=8.0, 1H), 6.16 (dd, J=2.4, 8.0, 1H), 6.11 (dd, J=2.3, 7.7, 1H), 5.25-4.30 (s, 1H), 2.88 (s, 3H).

### Synthesis of 4-fluoro-N-methylpyridin-2-amine (I-40)

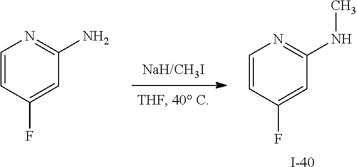

The above intermediate was prepared following the procedure outlined for the synthesis of 6-chloro-N-methylpyridin-3-amine.

### N-methylpyridin-3-amine (I-41)

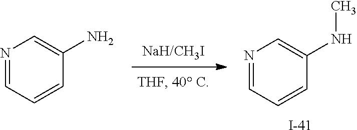

The above intermediate was prepared following the procedure outlined for the synthesis of 6-chloro-N-methylpyridin-3-amine. ¹H NMR (400 MHz, CDCl3) δ 8.00 (d, J=2.7, 1H), 7.92 (d, J=4.6, 1H), 7.07 (dd, J=4.6, 8.3, 1H), 6.84 (ddd, J=1.3, 2.9, 8.3, 1H), 2.83 (s, 3H).

### N-methyl-6-(trifluoromethyl)pyridin-3-amine (I-42)

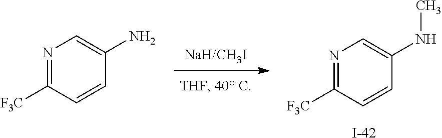

The above intermediate was prepared following the procedure outlined for the synthesis of 6-chloro-N-methylpyridin-3-amine. ¹H NMR (400 MHz, CDCl3) δ 8.04 (d, J=2.7, 1H), 7.45 (d, J=8.6, 1H), 6.85 (d, J=8.6, 1H), 4.34-3.88 (m, 1H), 2.89 (s, 3H).

### Synthesis of 2,6-dichloro-N-methylpyridin-3-amine (I-43)

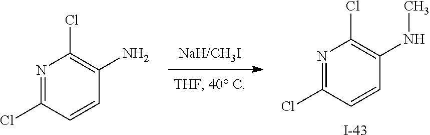

The above intermediate was prepared following the procedure outlined for the synthesis of 6-chloro-N-methylpyridin-3-amine. ¹H NMR (400 MHz, CDCl3) δ 7.11 (d, J=8.1, 1H), 6.84 (d, J=8.4, 1H), 4.38 (s, 1H), 2.88 (d, J=5.2, 3H).

### Synthesis of 6-bromo-N-methylpyridin-3-amine (I-44)

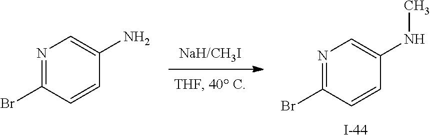

The above intermediate was prepared following the procedure outlined for the synthesis of 6-chloro-N-methylpyridin-3-amine. ¹H NMR (400 MHz, CDCl3) δ 7.74 (d, J=3.1, 1H), 7.20 (d, J=8.6, 1H), 6.75 (dd, J=3.1, 8.6, 1H), 2.82 (s, 3H).

### Synthesis of 6-bromo-N-methylpyridin-3-amine (I-45)

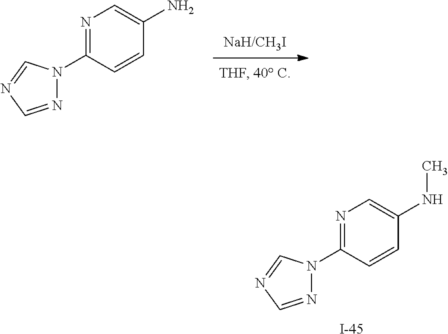

The above intermediate was prepared following the procedure outlined for the synthesis of 6-chloro-N-methylpyridin-3-amine. ¹H NMR (400 MHz, CDCl3) δ 8.97 (s, 1H), 8.02 (s, 1H), 7.80 (d, J=2.8, 1H), 7.68 (d, J=8.5, 1H), 7.03 (dd, J=2.9, 8.8, 1H), 2.90 (s, 3H).

### Synthesis of 2-methoxy-N-methylpyridin-3-amine (I-46)

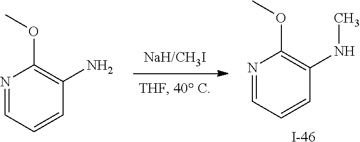

The above intermediate was prepared following the procedure outlined for the synthesis of 6-chloro-N-methylpyridin-3-amine. ¹H NMR (400 MHz, CDCl3) δ 7.46 (dd, J=1.6, 5.1, 1H), 6.77 (dd, J=5.1, 7.6, 1H), 6.67 (dd, J=1.5, 7.6, 1H), 4.17 (s, 1H), 3.95 (s, 3H), 2.82 (d, J=4.2, 3H).

### Synthesis of 2-fluoro-N-methylpyridin-3-amine (I-47)

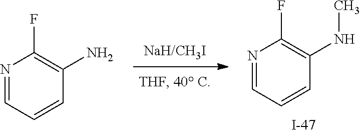

The above intermediate was prepared following the procedure outlined for the synthesis of 6-chloro-N-methylpyridin-3-amine. ¹H NMR (400 MHz, CDCl3) δ 7.47-7.35 (m, 1H), 6.57-6.29 (m, 2H), 3.09 (s, 4H).

### Synthesis of 6-chloro-N,5-dimethylpyridin-3-amine (I-48)

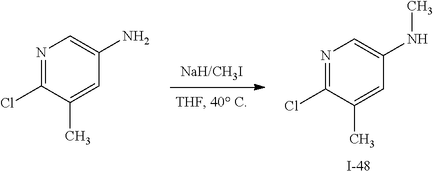

The above intermediate was prepared following the procedure outlined for the synthesis of 6-chloro-N-methylpyridin-3-amine. ¹H NMR (400 MHz, CDCl3) δ 7.60 (d, J=2.9, 1H), 6.75 (d, J=2.6, 1H), 2.81 (s, 3H), 2.28 (s, 3H).

### Synthesis of 5-chloro-N-methylpyridin-3-amine (I-49)

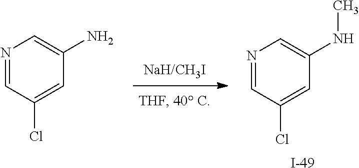

The above intermediate was prepared following the procedure outlined for the synthesis of 6-chloro-N-methylpyridin-3-amine. ¹H NMR (400 MHz, CDCl3) δ 7.94 (d, J=5.5, 1H), 6.55 (dd, J=1.7, 5.5, 1H), 6.35 (d, J=1.7, 1H), 4.75 (s, 1H), 2.88 (d, J=5.2, 3H).

### Synthesis of 2-chloro-N,5-dimethylpyridin-3-amine (I-50)

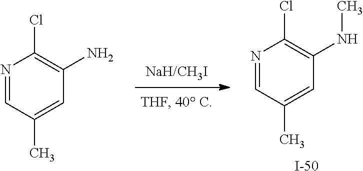

The above intermediate was prepared following the procedure outlined for the synthesis of 6-chloro-N-methylpyridin-3-amine. ¹H NMR (400 MHz, CDCl3) δ 7.51 (s, 1H), 6.66 (s, 1H), 4.26 (s, 1H), 2.86 (s, 3H), 2.25 (s, 3H).

### Synthesis of 5-fluoro-N-methylpyridin-3-amine (I-51)

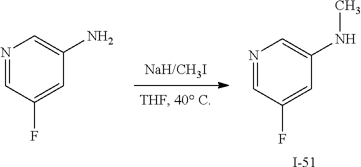

The above intermediate was prepared following the procedure outlined for the synthesis of 6-chloro-N-methylpyridin-3-amine. ¹H NMR (400 MHz, CDCl3) δ 7.82 (s, 1H), 7.78 (d, J=2.3, 1H), 6.54 (dt, J=2.4, 11.1, 1H), 3.99 (s, 1H), 2.83 (s, 3H).

### Synthesis of 6-fluoro-N-methylpyridin-3-amine (I-52)

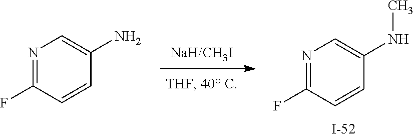

The above intermediate was prepared following the procedure outlined for the synthesis of 6-chloro-N-methylpyridin-3-amine. ¹H NMR (400 MHz, DMSO) δ 7.46-7.36 (m, 1H), 7.11 (ddd, J=3.1, 7.1, 8.8, 1H), 6.89 (dd, J=3.3, 8.8, 1H), 5.81 (s, 1H), 2.68 (d, J=5.2, 3H).

### Synthesis of N-methyl-6-phenylpyridin-3-amine (I-53)

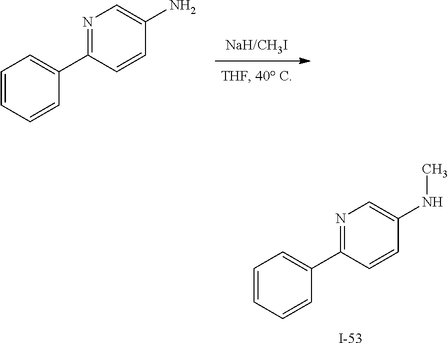

The above intermediate was prepared following the procedure outlined for the synthesis of 6-chloro-N-methylpyridin-3-amine. ¹H NMR (400 MHz, CDCl3) δ 8.10 (d, J=2.9, 1H), 7.91-7.85 (m, 2H), 7.56 (d, J=8.6, 1H), 7.44-7.37 (m, 2H), 7.33-7.26 (m, 1H), 6.93 (dd, J=3.0, 8.6, 1H), 3.85 (s, 1H), 2.88 (s, 3H).

### Synthesis of N-methyl-2-(trifluoromethyl)pyridin-4-amine (I-54)

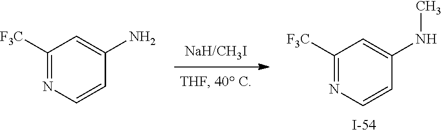

The above intermediate was prepared following the procedure outlined for the synthesis of 6-chloro-N-methylpyridin-3-amine. ¹H NMR (400 MHz, DMSO) δ 8.16 (d, J=5.7, 1H), 7.10 (d, J=4.3, 1H), 6.86 (s, 1H), 6.65 (dd, J=2.2, 5.7, 1H), 2.76 (d, J=5.0, 3H).

### Synthesis of 5-chloro-N-methylpyrazin-2-amine (I-55)

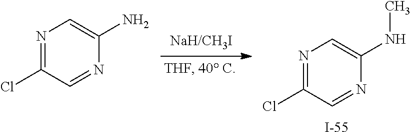

The above intermediate was prepared following the procedure outlined for the synthesis of 6-chloro-N-methylpyridin-3-amine. ¹H NMR (400 MHz, DMSO) δ 8.05 (d, J=1.4, 1H), 7.72 (d, J=1.4, 1H), 7.26 (s, 1H), 2.77 (d, J=4.7, 3H).

### Synthesis of 2,2-dimethyl-3,4-dihydro-2H-benzo[b][1,4]oxazine-7-carbonitrile (I-56)

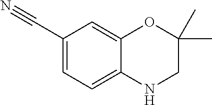

A flask was charged with bromobenoxazine and CuCN. The flask was evacuated and refilled with nitrogen three times. The flask was evacuated once more, sealed, and heated to 150° C. for 6 hours. The reaction was cooled to room temperature. 1 mL of 10% FeCl₃ in water was added followed by 1 mL of ethyl acetate. The reaction mixture was stirred for 10 minutes and then filtered through celite. The filtrate was further diluted with water and extracted with EtOAc (3×). The combined organic extracts were again filtered through celite. The filtrate was dried over MgSO₄ and concentrated to a black oil. TLC analysis showed two major spots, with the top spot corresponding to the starting material and the lower spot corresponding to the product. The reaction was purified by flash chromatography (silica, 0-80% ethyl acetate/hexanes). ¹H NMR (400 MHz, DMSO) δ 7.08 (dd, J=1.9, 8.2, 1H), 7.01-6.90 (m, 2H), 6.64 (d, J=8.2, 1H), 3.10 (d, J=2.6, 2H), 2.51 (dd, J=1.8, 3.6, 2H), 1.24 (s, 6H).

### Synthesis of 3-bromo-N-(6-chloropyridin-3-yl)-N-methylimidazo[1,2-a]pyrazine-6-carboxamide (I-57)

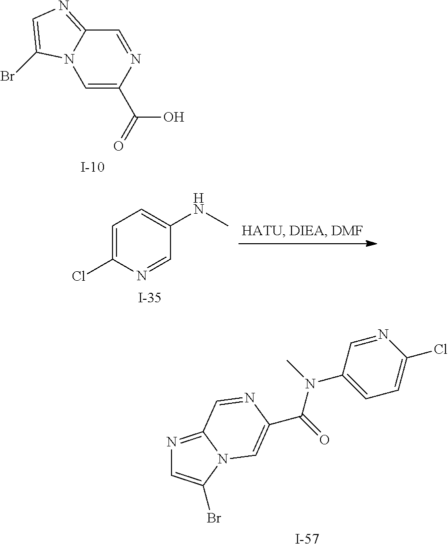

Into a 100-mL round-bottom flask, was placed a solution of I-10 (1.84 g, 7.60 mmol, 1.00 equiv) and 6-chloro-N-methylpyridin-3-amine (520 mg, 3.64 mmol, 1.00 equiv) in DMF(dry) (20 mL). Then HATU (2.1 g, 5.50 mmol, 1.50 equiv) and DIEA (470 mg, 3.64 mmol, 1.00 equiv) was added. The resulting solution was stirred for 12 h at room temperature. The reaction was then quenched by the addition of water. The solids were collected by filtration and then washed with water. This resulted in 1.2 g (42%) of 1-57 as a grey solid. ¹H-NMR-: (CDCl₃, ppm): δ 8.79-8.73 (d, J=1.5 Hz, 1H), 8.61 (s, 1H), 8.15-8.14 (d, J=2.1 Hz, 1H), 7.84 (s, 1H) 7.58-7.54 (m, 1H), 7.34-7.28 (m, 1H), 3.57 (s, 3H)

### Synthesis of tert-butyl(2-methyl-2-(4-(4,4,5,5-tetramethyl-1,3,2-dioxaborolan-2-yl)phenoxy)propyl)carbamate (I-58)

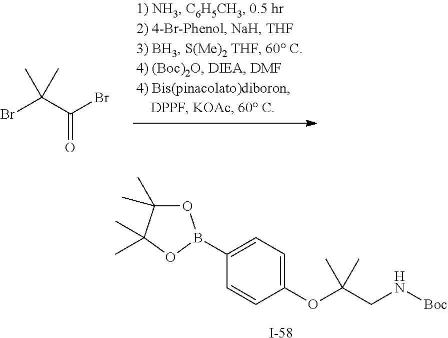

### Synthesis of 2-bromo-2-methylpropanamide

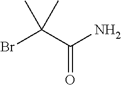

To a solution of 2-bromo-2-methylpropanoyl bromide (5 g, 21.81 mmol, 1 eq) in Toluene (10 mL) NH₃ gas was bubbled through for ½ hr. The reaction mixture was checked via LC/MS for the desired peak and full conversion and at this point the reaction mixture was neutralized with sodium bi-carbonate solution and extracted nice crystalline material (4 g, crude) which was used for the next step without further purification.

### Synthesis of 2-(4-bromophenoxy)-2-methylpropanamide

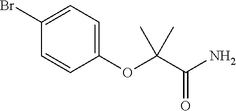

To a solution of the Bromo-phenol (1.30 g, 7.52 mmols 1.0 eq) pre-dissolved in THF, NaH (9.03 mmol, 1.5 eq,) was added. The reaction mixture was kept under reflux for 2 hrs. The 2-bromo-2-methylpropanamide (1.0 g, 6.02 mmol) predissolved in THF (10 mL) was added drop by drop. The reaction mixture was kept under reflux for 2 hrs. Quenched with water and extracted with EtOAC (3×50 mL). The organics were combined and dried over Na₂SO₄. The solvents were removed and the crude was further purified via column chromatography. Isolated 750 mg (2.9 mmol, 38%) of the desired product. M/Z=258 (M+1).

### Synthesis of 2-(4-bromophenoxy)-2-methylpropan-1-amine

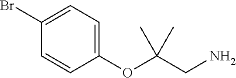

0.5 g (1.93 mmol, 1 eq) of 2-(4-bromophenoxy)-2-methylpropanamide was dissolved in dry THF (6 mL) and Diborane-DMS complex (2.90 mmol, 1.5 eq) was added slowly over ½ hr. The Reaction mixture was heated at reflux for 6 hrs. Checked via LC/MS and found the complete disappearance of the starting material and a nice product peak was found. The reaction mixture was diluted with EtOAc and sodium bicarbonate and the organics were extracted using EtOAc (3×15 mL). The desired compound was isolated by using column chromatography. M/Z=243 (M+1)

### Synthesis of tert-butyl(2-(4-bromophenoxy)-2-methylpropyl)carbamate

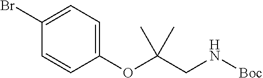 [SMILES: CC(C)(C)OC(=O)NCC(C)(C)Oc1ccc(Br)cc1]

To the solution of 2-(4-bromophenoxy)-2-methylpropan-1-amine in DMF (0.37 g, 1.51 mmol), added the (Boc)₂O (0.49 g, 2.26 mmols, 1.5 eq), followed by DIEA (1 mL). The reaction mixture was stirred for 12 hrs. The reaction was quenched with water, followed by extraction with EtOAc (3×10 mL). The desired compound was purified via ISCO to produce 300 mg (0.872 mmol, 58%) of the desired product.

### Synthesis of tert-butyl(2-methyl-2-(4-(4,4,5,5-tetramethyl-1,3,2-dioxaborolan-2-yl)phenoxy)propyl)carbamate

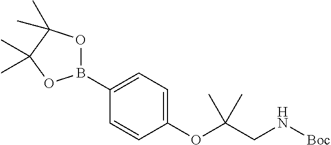 [SMILES: CC(C)(C)OC(=O)NCC(C)(C)Oc1ccc(B2OC(C)(C)C(C)(C)O2)cc1]

To a solution of tert-butyl(2-(4-bromophenoxy)-2-methylpropyl)carbamate (300 mg, 0.87 mmols, 1 eq), in THF/water (4:1) 6 mL was added 4,4,4′,4′,5,5,5′,5′-octamethyl-2,2′-bi(1,3,2-dioxaborolane) (330 mg, 1.30 mmol, 1.5 eq), followed by DPPF (40 mg), and KOAc (127 mg, 1.30 mmols, 1.5 eq). The microwave vial was sealed and heated at 110° C. for 1 hr. The desired product was isolated via column chromatography to produce (116 mg 34%, 0.296 mmol) of pure compound.

### Synthesis of 3-bromo-N-(4-cyanophenyl)-N-methylimidazo[1,2-a]pyrazine-6-carboxamide (I-59)

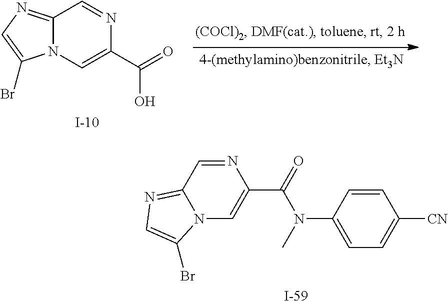

To a solution of 3-bromoimidazo[1,2-a]pyrazine-6-carboxylic acid 1-10 (4 g, 16.53 mmol, 1.00 equiv) in toluene (100 mL) was added dropwise oxalyl chloride (4.2 g, 33.09 mmol, 2.00 equiv) and 2-3 drops of DMF at room temperature. The solution was stirred for 2 hours at 55° C. Then the solvent was removed under vacuum, the residue was dissolved in dichloromethane (200 ml). To this was added triethylamine (8.3 g, 82.18 mmol, 5.00 equiv), 4-(methylamino)benzonitrile (2.4 g, 18.16 mmol, 1.10 equiv). The resulting solution was stirred for 1 hour at room temperature. The resulting solution was diluted with dichloromethane (100 mL), washed, dried and concentrated. The residue was applied onto a silica gel column with petroleum ether/dichloromethane/ethyl acetate (2/1/1). This resulted in 3.8 g (65%) of 3-bromo-N-(4-cyanophenyl)-N-methylimidazo[1,2-a]pyrazine-6-carboxamide as a light yellow solid. ¹H-NMR (CDCl₃, 300 MHz): 8.75 (d, J=1.5 Hz, 1H), 8.59-8.57 (dd, J=1.2, 5.4 Hz, 1H), 7.83 (s, 1H), 7.60-7.55 (m, 2H), 7.25-7.21 (m, 2H), 3.58 (s, 3H)

### Synthesis of N-methyl-5-(4-(4,4,5,5-tetramethyl-1,3,2-dioxaborolan-2-yl)phenyl)-1,3,4-oxadiazol-2-amine (I-60)

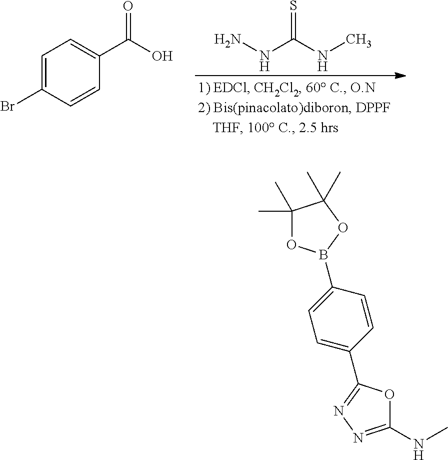

### Synthesis of 5-(4-bromophenyl)-N-methyl-1,3,4-oxadiazol-2-amine

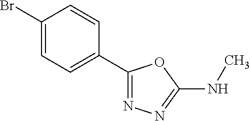

To the solution of 4-bromobenzoic acid (10 g, 49.75 mmol, 1 eq) in dichloromethane (100 mL) was added N-methylhydrazine carbothioamide (5.22 g, 49.75 mmol, 1.02 eq) followed by EDCl (14.72 g, 74.62 mmol, 1.5 eq). The reaction was stirred at room temperature for 12 hrs and checked via LC/MS for the desired amide intermediate peak. At this time another 1.5 eq of EDCl (14.72 g, 74.62 mmol) was added to the reaction mixture and stirred for 24 hrs at room temperature. The reaction was quenched with saturated aqueous sodium bicarbonate (20 mL), and then extracted with ethyl acetate (3×25 mL). The resulting crude material was purified by flash column chromatography on silica gel using 60% EtOAc/hexanes as eluant. M/Z=254 (M+1) RT 1.58 min using method 5.

### Synthesis of N-methyl-5-(4-(4,4,5,5-tetramethyl-1,3,2-dioxaborolan-2-yl)phenyl)-1,3,4-oxadiazol-2-amine

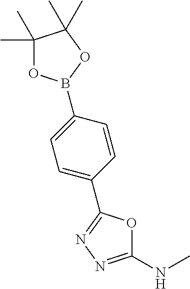

To the solution of 5-(4-bromophenyl)-N-methyl-1,3,4-oxadiazol-2-amine (1.8 g, 7.08 mmol, 1 eq) in THF/Water (4:1), was added 4,4,4′,4′,5,5,5′,5′-octamethyl-2,2′-bi(1,3,2-dioxaborolane) (2.24 g, 8.85 mmols, 1.25 eq.) followed by KOAc (1.73 g, 17.70 mmol, 2.5 eq), and DPPF (518 mg, 0.1 eq, 0.71 mmol). The microwave vial was sealed and heated at 100° C. for 2.5 hrs. Checked via LC/MS for the desired peak and then the desired product was isolated using column chromatography. The desired product was isolated off white solid (461 mg, 1.51 mmol, 21%) was used for the next step. ¹H NMR (400 MHz, MeOD) δ 8.02-7.76 (m, 4H), 3.00 (s, 3H), 1.37 (s, 12H).

### Synthesis of 2-(4-(4,4,5,5-tetramethyl-1,3,2-dioxaborolan-2-yl)phenyl)-1,3,4-thiadiazole (I-61)

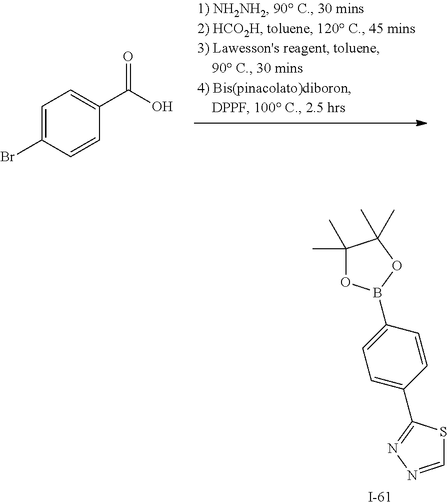

### Synthesis of 4-Bromobenzohydrazide

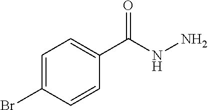 [SMILES: NNC(=O)c1ccc(Br)cc1]

4-bromobenzoic acid (500 mg, 2.48 mmol, 1 eq.) was added to hydrazine-hydrate (10 g, 50 mmol, 81 eq), and the reaction mixture was heated under microwave irradiation condition at 90° C. for 30 minutes. The solvents were removed and the crude was used for the next step. (M/Z) 214 (M+1). RT 1.07 min using method 5.

### Synthesis of 4-bromo-N′-formylbenzohydrazide

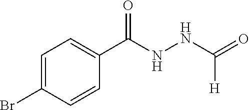 [SMILES: O=CNNC(=O)c1ccc(Br)cc1]

To a solution of 4-bromohydrazide (460 mg, 2.15 mmol, 1 eq) in toluene (12 mL) formic acid (1.5 g, 32.6 mmol, 15 eq) was added drop-wise over 10 minute and the reaction mixture was heated under microwave irradiation condition at 120° C. for 90 minutes. The product was isolated and purified via ISCO column chromatography. Used for the next step. (M/Z) 244 (M+1). RT 1.77 min method 5

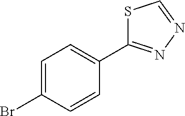 [SMILES: Brc1ccc(-c2nncs2)cc1]

To a solution of 4-bromo-N′-formylbenzohydrazide (400 mg, 1.65mmols, 1 eq) dissolved in toluene (12 mL) Lawesson's reagent (1 g, 2.47 mmol, pre-dissolved in toluene (5 mL) was added. The reaction mixture was heated under microwave irradiation at 90° C. for 30 minutes. Isolated product was used for the next step. (M/Z) 241 (M+H). RT 1.84 min using method 5.

### Synthesis of 2-(4-(4,4,5,5-tetramethyl-1,3,2-dioxaborolan-2-yl)phenyl)-1,3,4-thiadiazole

The compound was synthesized in using a procedure similar to one described for I-59. (M/Z) 289 (M+1). RT 1.66 min using method 5.

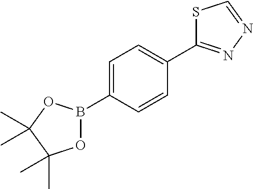 [SMILES: CC1(C)OB(c2ccc(-c3nncs3)cc2)OC1(C)C]

### Synthesis of N-((3-bromoimidazo[1,2-a]pyrazin-6-yl)methyl)-4-fluoro-N-methylaniline (I-62)

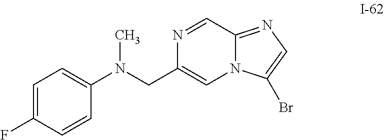 [SMILES: CN(Cc1cn2c(Br)cnc2cn1)c1ccc(F)cc1]

The compound was synthesized using a protocol described for the synthesis of 1-15.

### Synthesis of N-((3-bromoimidazo[1,2-a]pyrazin-6-yl)methyl)-4-chloro-N-methylaniline (I-63)

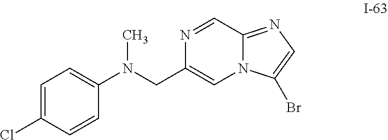 [SMILES: CN(Cc1cn2c(Br)cnc2cn1)c1ccc(Cl)cc1]

The compound was synthesized using a protocol described for the synthesis of I-15.

### Synthesis 3-bromo-N-(4-fluorophenyl)-N-methylimidazo[1,2-a]pyrazine-6-carboxamide (I-64)

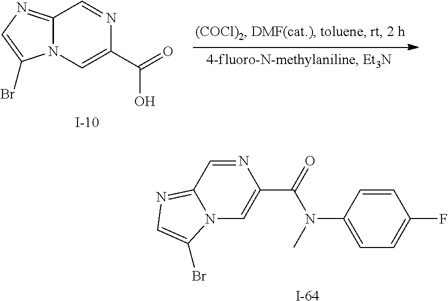

To a solution of 3-bromoimidazo[1,2-a]pyrazine-6-carboxylic acid (4 g, 16.53 mmol, 1.00 equiv) in toluene (100 mL) was added dropwise oxalyl dichloride (4.2 g, 33.09 mmol, 2.00 equiv) at room temperature. The solution was stirred for 2 hours at 55° C. Then the solvent was removed under vacuum, the residue was dissolved dichloromethane (200 ml). To this was added triethylamine (8.3 g, 82.18 mmol, 5.00 equiv), 4-fluoro-N-methylbenzenamine (2.27 g, 18.16 mmol, 1.10 equiv). The resulting solution was stirred for 1 hour at room temperature. The resulting solution was diluted with DCM (100 mL), washed and dried. The residue was applied onto a silica gel column with petroleum ether/dichloromethane/ethyl acetate (2/1/1). This resulted in 3.8 g (67%) of 3-bromo-N-(4-fluorophenyl)-N-methylimidazo[1,2-a]pyrazine-6-carboxamide as a light yellow solid. ¹H-NMR (CDCl₃, 300 MHz): 8.62 (brs, 2H), 7.80 (s, 1H), 7.11 (brs, 2H), 6.96 (t, J=5.4 Hz, 2H), 3.52 (s, 3H)

### Synthesis 3-bromo-N-(3,4-difluorophenyl)-N-methylimidazo[1,2-a]pyrazine-6-carboxamide (I-65)

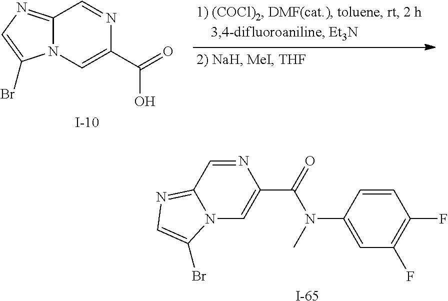

Into a 250-mL round-bottom flask, was placed a solution of 3-bromoimidazo[1,2-a]pyrazine-6-carboxylic acid (2 g, 8.26 mmol, 1.00 equiv) in dichloromethane (60 mL). Then oxalyl dichloride (5.3 g, 41.73 mmol, 5.05 equiv) and several drops of DMF were added. The resulting solution was stirred for 60 min at room temperature. Concentrated under vacuum to remove excess oxalyl dichloride and freshed dichloromethane (60 mL) was added. Trifluorobenzenamine (3.2 g, 24.81 mmol, 3.00 equiv), triethylamine (10 g, 99.01 mmol, 11.98 equiv) was added. The resulting solution was allowed to react, with stirring, overnight at room temperature. The resulting mixture was concentrated under vacuum. The residue was applied onto a silica gel column with ethyl acetate/petroleum ether (1:2-1:1). This resulted in 1.6 g (55%) of 3-bromo-N-(3,4-difluorophenyl)imidazo[1,2-a]pyrazine-6-carboxamide as a white solid.

Into a 100-mL 3-necked round-bottom flask purged and maintained with an inert atmosphere of nitrogen, was placed a solution of 3-bromo-N-(3,4-difluorophenyl)imidazo[1,2-a]pyrazine-6-carboxamide (1.6 g, 4.53 mmol, 1.00 equiv) in N,N-dimethylformamide (30 mL). This was followed by the addition of sodium hydride (400 mg, 11.67 mmol, 2.57 equiv, 70%) at room temperature. The resulting solution was stirred for 1 h at room temperature. To this was added iodomethane (4 g, 28.17 mmol, 6.21 equiv) at room temperature. The resulting solution was allowed to react, with stirring, for an additional 1.5 h at room temperature. The reaction was then quenched by the addition of 50 mL of water. The resulting solution was extracted with 2×200 mL of ethyl acetate and the organic layers were combined, washed with 4×200 mL of brine, dried over anhydrous sodium sulfate and concentrated under vacuum. The residue was applied onto a silica gel column with Petroleum Ether:EtOAc (2:1-1:1). This resulted in 1.2 g (70%) of I-65 as a yellow solid. ¹H NMR (300 MHz, CDCl₃) δ 8.70 (s, 1H), 8.65 (s, 1H), 7.82 (s, 1H), 7.06 (m, 2H), 6.86 (s, 1H).

### Synthesis 3-bromo-N-(4-fluorophenyl)-N-methylimidazo[1,2-a]pyrazine-6-carboxamide (I-66)

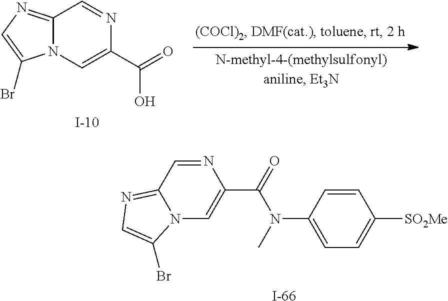

The compound was synthesized using a protocol as described for I-64.

### Synthesis of tert-butyl(2-methyl-2-(4-(4,4,5,5-tetramethyl-1,3,2-dioxaborolan-2-yl)phenoxy)propanoyl)carbamate (I-67)

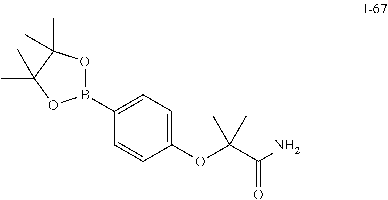 [SMILES: CC(C)(Oc1ccc(B2OC(C)(C)C(C)(C)O2)cc1)C(N)=O]

The compound was synthesized using a protocol as described for I-58.

### Synthesis of 5-cyclobutyl-N-methylpyridin-2-amine (I-68)

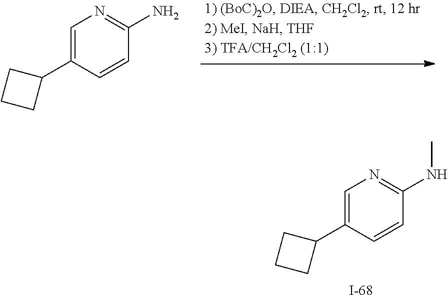

General Procedure: To a solution of the 5-cyclobutylpyridin-2-amine (1 eq) in CH₂Cl₂ was added the Boc-anhydride (1.25 eq) followed by DIEA. The reaction mixture was stirred at room temp. for 12 hours. The solvents were removed under vacuo, extracted the organics with ethyl acetate and the crude was used for the next step without further purifications. The crude yields are 50-90% in most cases (different substrates).

The solution of tert-butyl(5-cyclobutylpyridin-2-yl)carbamate was dissolved in dry THF and Mel, followed by NaH was added to the reaction mixture. The reaction mixture was stirred at room temp for 2 hrs and the checked via LC/MS for the desired peak. The crude was extracted and redissolved in TFA/DCM (1:1) and stirred for ½ hr. The reaction mixture was quenched with sodium carbonate solution and concentrated before using for the next step. Method B M/Z=163 (M+1), RT 1.18 min

### Synthesis of 6-Fluoro-1,2,3,4-tetrahydroquinoline (I-69)

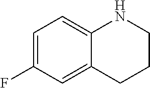 [SMILES: Fc1ccc2c(c1)CCCN2]

### Step 1: 3-Chloro-N-(4-fluorophenyl)propanamide

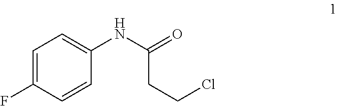 [SMILES: O=C(CCCl)Nc1ccc(F)cc1]

2-Chloropropionyl chloride (2.14 mL, 22.45 mmol) was added to a solution of 4F-aniline (4.27 mL, 45.05 mmol) in dichloromethane (200 mL) at 0° C. and stirred at ambient temperature for 1 hour. To the reaction mass was added water and the organic layer was washed with sat. NaHCO₃, brine, dried over Na₂SO₄ and concentrated under reduced pressure to afford 4.0 g (45% crude yield) of 3-chloro-N-(4-fluorophenyl)propanamide 1 as a light blue solid. ¹H-NMR (CDCl₃): δ 7.44-7.48 (m, 3H), 7.01 (t, J=8.35 Hz, 2H), 3.87 (t, J=6.59 Hz, 2H), 2.80 (t, J=6.15 Hz, 2H).

### Step 2: 6-Fluoro-3,4-dihydroquinolin-2(1H)-one

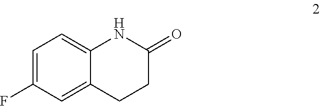 [SMILES: O=C1CCc2cc(F)ccc2N1]

To a suspension of 3-chloro-N-(4-fluorophenyl)propanamide 1 (2.0 g, 9.95 mmol) and AlCl₃ (4.6 g, 34.82 mmol) was stirred at 120 t for 3 h. Cold water was added to the reaction mass under vigorous stirring and neutralized with 6N HCl at 0° C. The solid was collected by filtration, washed with water and dried to afford 1.4 g (85%) of 6-fluoro-3,4-dihydroquinolin-2(1H)-one 2 as a brown solid. ¹H NMR (400 MHz, CDCl₃): δ 6.85-6.90 (m, 2H), 6.72-6.75 (m, 1H), 2.95 (t, J=7.91 Hz, 2H), 2.62 (t, J=7.03 Hz, 2H).

### Step 3: 6-Fluoro-1,2,3,4-tetrahydroquinoline

To a suspension of LiAlH₄ (2.76 g, 72.73 mmol) THF (80 mL) was added a solution of 6-fluoro-3,4-dihydroquinolin-2(1H)-one 2 (6.0 g, 36.36 mmol) in THF at stirred at ambient temperature for 2 h. The reaction was quenched with brine and ethyl acetate and filtered through celite. The filtrate was diluted with ethyl acetate, washed with water, brine and dried over anhyd. Na₂SO₄ and concentrated to gave 4.5 g (81%) of 6-fluoro-1,2,3,4-tetrahydroquinoline as a brown color liquid. ¹H NMR (400 MHz, CDCl₃): δ 6.65-6.78 (m, 2H), 6.37-6.40 (m, 1H), 3.26 (t, J=5.3 Hz, 2H), 2.73 (t, J=6.6 Hz, 2H), 1.88-1.94 (m, 2H).

### Synthesis of 6-Fluoro-2,2-dimethyl-1,2,3,4-tetrahydroquinoline (I-70)

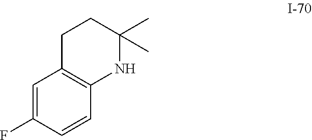

### Step 1: 1-Benzyl-6-fluoro-3,4-dihydroquinolin-2(1H)-one (1a)

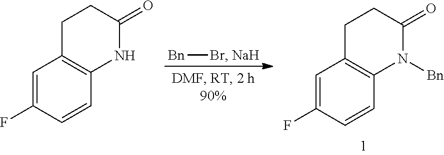

To a cold solution (0° C.) of compound quinolone derivative (5 g, 30.3 mmol) in DMF (100 mL) was added NaH (60%) (1.98 g, 45.4 mmol) and stirred at 0° C. for 20 min. To the resulting solution benzyl chloride (4.2 mL, 36.3 mmol) was added and stirred at rt for 2 h. The reaction was quenched with water (50 mL) and extracted with ethyl acetate (2×60 mL). The combined organic layer was washed with brine (20 mL), dried over Na₂SO₄ and concentrated in vacuo to obtain 7.0 g (90%) of 1-benzyl-6-fluoro-3,4-dihydroquinolin-2(1H)-one 1 as a brown color liquid. ¹H NMR (400 MHz, CDCl₃): δ 7.29-7.32 (m, 2H), 7.18-7.26 (m, 3H), 6.75-6.90 (m, 3H) 5.16 (s, 2H), 2.96 (t, J=7.9 Hz, 2H), 2.76-2.80 (m, 2H).

### Step 2. 1-Benzyl-6-fluoro-2,2-dimethyl-1,2,3,4-tetrahydroquinoline (2)

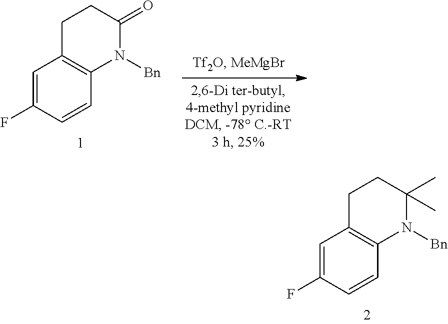 [SMILES: O=C1CCc2cc(F)ccc2N1Cc1ccccc1 ; CC1(C)CCc2cc(F)ccc2N1Cc1ccccc1]

### Brief Procedure:

To a cold solution (−78° C.) of 1-benzyl-6-fluoro-3,4-dihydroquinolin-2(1H)-one 1 (3.8 g, 14.9 mmol) in dichloromethane (400 mL) was added 2,6-di-ter-butylpyridine (3.66 g, 17.88 mmol) followed by Tf₂O (10.5 mL, 37.25 mmol) and stirred at −78° C. for 45 min. Then MeMgBr (3M in Et₂O, 29.7 mL, 89.4 mmol) was added at −78° C. and stirred at ambient temperature for 3 h. The reaction was quenched with aq. NH₄Cl (100 mL) and extracted with dichloromethane (2×200 mL). The combined organic layer was washed with brine (100 mL), dried over Na₂SO₄ and concentrated under reduced pressure. The crude compound was purified by column chromatography over silica gel (100-200 mesh) using a solvent gradient of 1% EtOAc-pet ether as eluant to afford 985 mg (25%) of 1-benzyl-6-fluoro-2,2-dimethyl-1,2,3,4-tetrahydroquinoline 2 as a pale yellow liquid. LC-MS: m/z 270.14 (M+H) with a purity of 94.04%. ¹H NMR (400 MHz, CDCl₃): δ 7.20-7.33 (m, 5H), 6.71-74 (m, 1H), 6.57-6.62 (m, 1H), 6.15-6.19 (m, 1H), 4.43 (s, 2H), 2.83 (t, J=6.59 Hz, 2H), 1.91 (t, J=6.59 Hz, 2H), 1.24 (s, 6H).

### Step 3: 6-Fluoro-2,2-dimethyl-1,2,3,4-tetrahydroquinoline

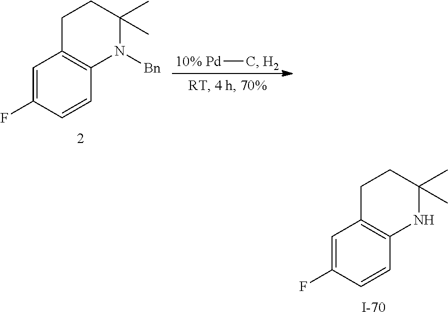

To a solution of 1-benzyl-6-fluoro-2,2-dimethyl-1,2,3,4-tetrahydroquinoline 2 (1.2 g, 4.46 mmol) in ethanol (100 mL) was added 10% Pd/C (300 mg) and hydrogenated under Paar hydrogenator at 40 psi for 6 h. The reaction mixture was filtered through celite and concentrated under reduced pressure. The crude compound was purified by column chromatography over silica gel (100-200 mesh) using a solvent gradient of 5% EtOAc-pet ether as eluent to afford 565 mg (70%) of 6-fluoro-2,2-dimethyl-1,2,3,4-tetrahydroquinoline I-70 as a pale yellow liquid. ¹H NMR (400 MHz, CDCl₃): δ 6.67-6.74 (m, 2H), 6.41-6.44 (m, 1H), 2.75 (t, J=7.0 Hz, 2H), 1.68 (t, J=7.0 Hz, 2H), 1.20 (s, 6H).

### Synthesis of (3-Bromoimidazo[1,2-a]pyrazin-6-yl)(6-fluoro-2,2-dimethyl-3,4-dihydroquinolin-1(2H)-yl)methanone I-71

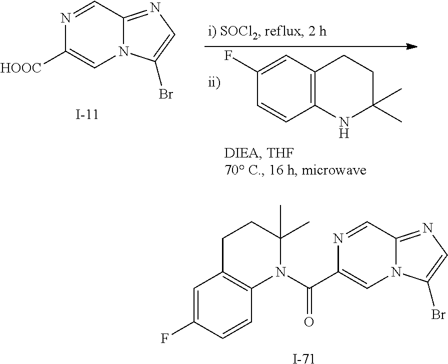

A mixture of 3-bromoimidazo[1,2-a]pyrazine-6-carboxylic acid 3 (200 mg, 0.829 mmol, 1 equiv) in SOCl₂ (6 mL) was refluxed for 2 h. Excess of SOCl₂ was distilled-off and the residue was dissolved in THF (20 mL). To the resulting solution were added a solution of 6-fluoro-2,2-dimethyl-1,2,3,4-tetrahydroquinoline I-70 (223 mg, 1.24 mmol, 1.5 equiv) and DIPEA (320 mg, 2.48 mmol, 3 equiv) in THF (2 mL) at 0° C. and stirred at 70° C. for 14 h under microwaves. The reaction mixture was concentrated and to the residue water (20 mL) was added and extracted with EtOAc (2×50 mL). The combined organic layer was washed with brine (20 mL), dried over Na₂SO₄ and concentrated under reduced pressure. The crude compound was purified by column chromatography over silica gel (100-200 mesh) using a solvent gradient of 10% EtOAc-pet ether as eluant to afford 50 mg (15%) of (3-bromoimidazo[1,2-a]pyrazin-6-yl)(6-fluoro-2,2-dimethyl-3,4-dihydroquinolin-1(2H)-yl)methanone I-71 as a Gummy solid. ¹H NMR (400 MHz, DMSO-d₆): δ 8.68 (s, 1H), 8.62 (s, 1H), 8.02 (s, 1H), 7.02-7.08 (m, 1H), 6.67-6.64 (m, 1H), 6.58-6.53 (m, 1H), 2.69 (t, 2H), 1.78 (br. s, 2H), 1.65 (s, 6H).

### Synthesis of (3-bromoimidazo[1,2-a]pyrazin-6-yl)(6-fluoro-3,4-dihydroquinolin-1(2H)-yl)methanone I-72

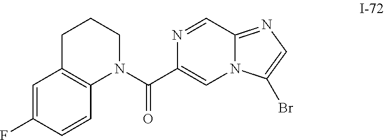 [SMILES: O=C(c1cn2c(Br)cnc2cn1)N1CCCc2cc(F)ccc21]

The compound was synthesized using a protocol described for the synthesis of I-71 using the intermediate I-69.

### Synthesis of Fluoro-3,4-dihydro-2H-benzo[b][1,4]oxazine I-73

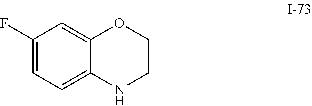 [SMILES: Fc1ccc2c(c1)OCCN2]

A suspension of 2-amino-5-fluorophenol (5.0 g, 39.37 mmol), 1,2-dibromoethane (21.9 g, 118.15 mmol), and potassium carbonate (27.16 g, 196.8 mmol) in DMF (50 mL) was stirred at 100° C. for 6 h. The reaction was diluted with water and extracted the product into ethyl acetate. The combined organic layer was washed with water, brine, dried over anhyd. Na₂SO₄ and concentrated in vacuo. The residue was purified by column chromatography over silica gel (100-200 mesh) with 10% ethyl acetate in pet-ether as eluant gave 2.6 g (43%) of 7-fluoro-3,4-dihydro-2H-benzo[b][1,4]oxazine I-73 as a brown solid. ¹H NMR (400 MHz, CDCl₃): 6.46-6.55 (m, 3H), 4.24 (t, J=4.4 Hz, 2H), 3.61 (br. s, 1H), 3.38 (t, J=4.4 Hz, 2H).

### Synthesis of (3-bromoimidazo[1,2-a]pyrazin-6-yl)(6-fluoro-3,4-dihydroquinolin-1(2H)-yl)methanone I-74

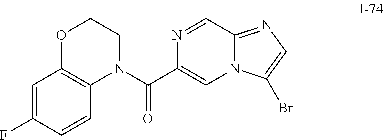 [SMILES: O=C(c1cn2c(Br)cnc2cn1)N1CCOc2cc(F)ccc21]

The compound was synthesized using a protocol described for the synthesis of I-71 using the intermediate I-73.

### Synthesis 3-bromo-N-(5-cyanopyridin-2-yl)-N-methylimidazo[1,2-a]pyrazine-6-carboxamide (I-75)

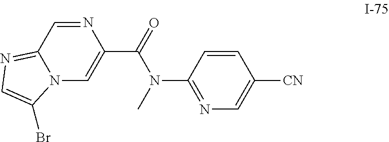 [SMILES: CN(C(=O)c1cn2c(Br)cnc2cn1)c1ccc(C#N)cn1]

The compound was synthesized using a protocol described for the synthesis of I-65 using the intermediate I-37.

### Synthesis of N-((3-bromoimidazo[1,2-a]pyrazin-6-yl)methyl)-6-chloro-N-methylpyridin-3-amine (I-76)

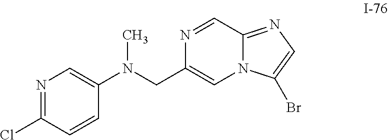 [SMILES: CN(Cc1cn2c(Br)cnc2cn1)c1ccc(Cl)nc1]

The compound was synthesized using a protocol described for the synthesis of I-15.

### Synthesis of N-((3-bromoimidazo[1,2-a]pyrazin-6-yl)methyl)-N,5-dimethylpyridin-2-amine (I-77)

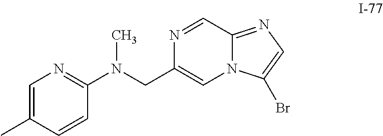 [SMILES: Cc1ccc(N(C)Cc2cn3c(Br)cnc3cn2)nc1]

The compound was synthesized using a protocol described for the synthesis of I-15.

### Synthesis of 3-bromo-N-methyl-N-(5-methylpyridin-2-yl)imidazo[1,2-a]pyrazine-6-carboxamide (I-78)

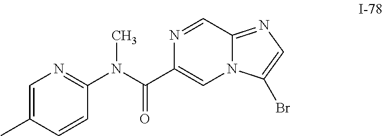 [SMILES: Cc1ccc(N(C)C(=O)c2cn3c(Br)cnc3cn2)nc1]

The compound was synthesized using a protocol described for the synthesis of I-15.

### Synthesis of Compounds

### Synthesis of Compound 1

### N-(4-cyanophenyl)-3-(6-(3-(dimethylamino)propoxy)pyridin-3-yl)-N-methylimidazo[1,2-a]pyrazine-6-carboxamide

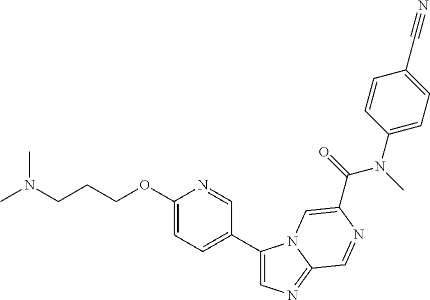 [SMILES: CN(C)CCCOc1ccc(-c2cnc3cnc(C(=O)N(C)c4ccc(C#N)cc4)cn23)cn1]

The compound was synthesized using I-59 and an appropriate boronic acid/ester using a procedure similar Suzuki coupling protocol described in 228. M/Z=456.20 (M+1), r.t=1.04 mins.

### Synthesis of Compound 2

### 4-(((3-(4-(1H-pyrazol-5-yl)phenyl)imidazo[1,2-a]pyrazin-6-yl)methyl)(methyl)amino)benzonitrile

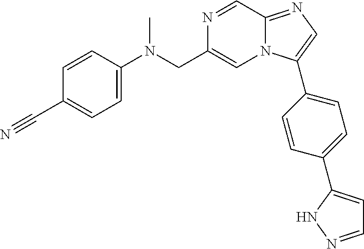 [SMILES: CN(Cc1cn2c(-c3ccc(-c4ccn[nH]4)cc3)cnc2cn1)c1ccc(C#N)cc1]

The compound was synthesized using I-15 and an appropriate boronic acid/ester using a procedure similar Suzuki coupling protocol described in 228. MS m/z 406.1 (M+H)+; r.t.=1.464.

### Synthesis of Compound 3

### N-((3-(4-(1H-pyrazol-5-yl)phenyl)imidazo[1,2-a]pyrazin-6-yl)methyl)-4-fluoro-N-methylaniline

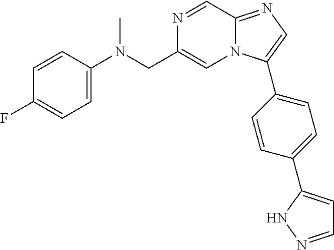 [SMILES: CN(Cc1cn2c(-c3ccc(-c4ccn[nH]4)cc3)cnc2cn1)c1ccc(F)cc1]

The compound was synthesized using I-62 and an appropriate boronic acid/ester using a procedure similar Suzuki coupling protocol described in 228. MS m/z 399.2 (M+H)+; r.t.=1.367

### Synthesis of Compound 4

### 4-(((3-(4-chlorophenyl)imidazo[1,2-a]pyrazin-6-yl)methyl)(methyl)amino)benzonitrile

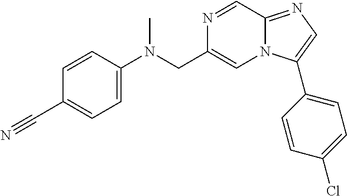 [SMILES: CN(Cc1cn2c(-c3ccc(Cl)cc3)cnc2cn1)c1ccc(C#N)cc1]

The compound was synthesized using I-63 and an appropriate boronic acid/ester using a procedure similar Suzuki coupling protocol described in 228. MS m/z 374.0 (M+H)+; r.t.=1.751

### Synthesis of Compound 5

### N-((3-(4-(1H-pyrazol-5-yl)phenyl)imidazo[1,2-a]pyrazin-6-yl)methyl)-4-chloro-N-methylaniline

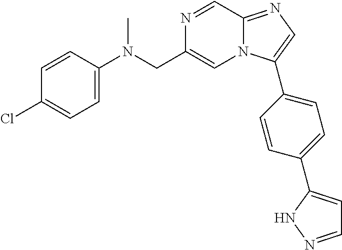 [SMILES: CN(Cc1cn2c(-c3ccc(-c4ccn[nH]4)cc3)cnc2cn1)c1ccc(Cl)cc1]

The compound was synthesized using I-63 and an appropriate boronic acid/ester using a procedure similar Suzuki coupling protocol described in 228. MS m/z 415.1 (M+H)+; r.t.=1.654.

### Synthesis of Compound 6

### N-(5-chloro-3-fluoropyridin-2-yl)-N-methyl-3-(4-(trifluoromethyl)phenyl)imidazo[1,2-a]pyrazine-6-carboxamide

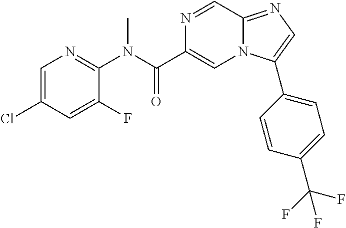 [SMILES: CN(C(=O)c1cn2c(-c3ccc(C(F)(F)F)cc3)cnc2cn1)c1ncc(Cl)cc1F]

The compound was synthesized using a procedure analogous to one use for synthesis of 72 (method II). MS m/z 450.0 (M+H)+; r.t.=1.908.

### Synthesis of Compound 7

### 5-methanesulfonyl-1-({3-[4-(trifluoromethyl)phenyl]imidazo[1,2-a]pyrazin-6-yl}carbonyl)-2,3-dihydro-1H-indole

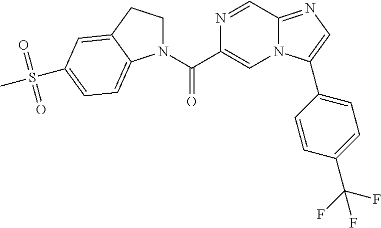 [SMILES: CS(=O)(=O)c1ccc2c(c1)CCN2C(=O)c1cn2c(-c3ccc(C(F)(F)F)cc3)cnc2cn1]

The compound was synthesized using a procedure similar to one used for the synthesis of 128. MS m/z 487.1 (M+H)+; r.t.=2.120.

### Synthesis of Compound 8

### 4-fluoro-N-methyl-N-((3-(1-methyl-1H-indazol-5-yl)imidazo[1,2-a]pyrazin-6-yl)methyl)aniline

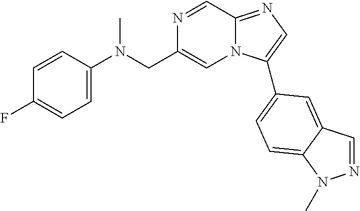 [SMILES: CN(Cc1cn2c(-c3ccc4c(cnn4C)c3)cnc2cn1)c1ccc(F)cc1]

The compound was synthesized using I-62 and an appropriate boronic acid/ester using a procedure similar Suzuki coupling protocol described in 228. MS m/z 387.1 (M+H)+; r.t.=1.466.

### Synthesis of Compound 9

### N-((3-(4-(1H-pyrazol-1-yl)phenyl)imidazo[1,2-a]pyrazin-6-yl)methyl)-4-chloro-N-methylaniline

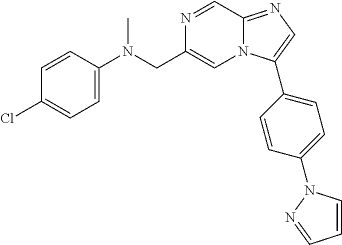 [SMILES: CN(Cc1cn2c(-c3ccc(-n4cccn4)cc3)cnc2cn1)c1ccc(Cl)cc1]

The compound was synthesized using I-63 and an appropriate boronic acid/ester using a procedure similar Suzuki coupling protocol described in 228. MS m/z 415.1 (M+H)+; r.t.=1.812.

### Synthesis of Compound 10

### N-((3-(4-(1H-pyrazol-1-yl)phenyl)imidazo[1,2-a]pyrazin-6-yl)methyl)-4-chloro-N-methylaniline

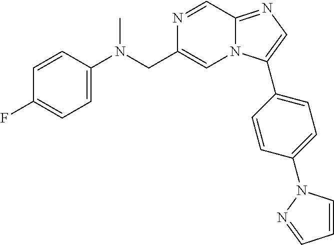 [SMILES: CN(Cc1cn2c(-c3ccc(-n4cccn4)cc3)cnc2cn1)c1ccc(F)cc1]

The compound was synthesized using I-62 and an appropriate boronic acid/ester using a procedure similar Suzuki coupling protocol described in 228. MS m/z 399.1 (M+H)+; r.t.=1.594.

### Synthesis of Compound 11

### 3-(4-carbamoylphenyl)-N-(4-fluorophenyl)-N-methylimidazo[1,2-a]pyrazine-6-carboxamide

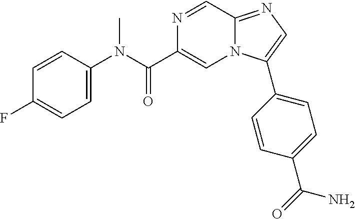 [SMILES: CN(C(=O)c1cn2c(-c3ccc(C(N)=O)cc3)cnc2cn1)c1ccc(F)cc1]

The compound was synthesized using I-64 and an appropriate boronic acid/ester using a procedure similar Suzuki coupling protocol described in 228. ¹H NMR (400 MHz, DMSO) δ 8.85 (s, 1H), 8.79 (s, 1H), 8.18 (s, 1H), 8.16 (s, 1H), 8.09 (d, J=8.4, 2H), 7.78 (d, J=8.3, 2H), 7.54 (s, 1H), 7.40-7.26 (m, 2H), 7.12 (t, J=8.6, 2H), 3.41 (s, 3H); MS m/z 390.1 (M+H)+; r.t.=1.168.

### Synthesis of Compound 12

### N-methyl-N-(1-methyl-1H-benzo[d][1,2,3]triazol-5-yl)-3-(4-(trifluoromethyl)phenyl)imidazo[1,2-a]pyrazine-6-carboxamide

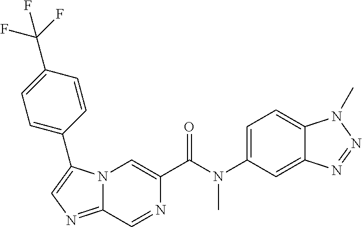 [SMILES: CN(C(=O)c1cn2c(-c3ccc(C(F)(F)F)cc3)cnc2cn1)c1ccc2c(c1)nnn2C]

The compound was synthesized using a procedure analogous to one use for synthesis of 72 (method II). MS m/z 452.1 (M+H)+; r.t.=1.696

### Synthesis of Compound 13

### N-(4-chlorophenyl)-N-methyl-3-[4-(1H-pyrazol-5-yl)phenyl]imidazo[1,2-a]pyrazine-6-carboxamide

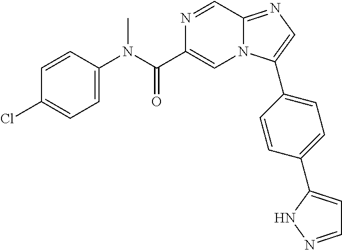 [SMILES: CN(C(=O)c1cn2c(-c3ccc(-c4ccn[nH]4)cc3)cnc2cn1)c1ccc(Cl)cc1]

The compound was synthesized using I-11 and an appropriate boronic acid/ester using a procedure similar Suzuki coupling protocol described in 228. MS m/z 429.0 (M+H)+; r.t.=1.519.

### Synthesis of Compound 14

### N-(1,3-benzothiazol-2-yl)-N-methyl-3-[4-(trifluoromethyl)phenyl]imidazo[1,2-a]pyrazine-6-carboxamide

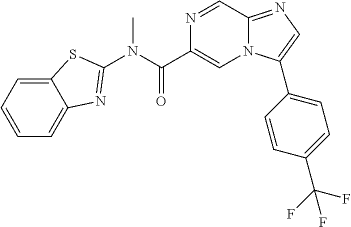 [SMILES: CN(C(=O)c1cn2c(-c3ccc(C(F)(F)F)cc3)cnc2cn1)c1nc2ccccc2s1]

The compound was synthesized using a procedure analogous to one use for synthesis of 72 (method III). 1H-NMR (400 MHz, CDCl₃) ppm 9.17 (s, 1H), 9.0 (s, 1H), 8.06 (s, 1H), 7.75-7.91 (m, 6H), 7.47 (m, 1H), 7.35 (m, 1H), 4.04 (s, 3H); LC-MS: 98.84%; 454.03 (M+H).

### Synthesis of Compound 15

### -[4-(5-amino-1,3,4-oxadiazol-2-yl)phenyl]-N-(4-chlorophenyl)-N-methylimidazo[1,2-a]pyrazine-6-carboxamide

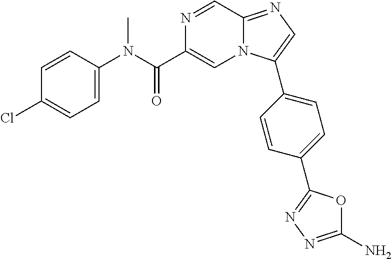 [SMILES: CN(C(=O)c1cn2c(-c3ccc(-c4nnc(N)o4)cc3)cnc2cn1)c1ccc(Cl)cc1]

The compound was synthesized using I-11 and I-24 using a procedure similar Suzuki coupling protocol described in 228. M/Z=446.1 (M+1), r.t=1.27 mins.

### Synthesis of Compound 16

### N-(3-methoxypyridin-2-yl)-N-methyl-3-[4-(trifluoromethyl)phenyl]imidazo[1,2-a]pyrazine-6-carboxamide

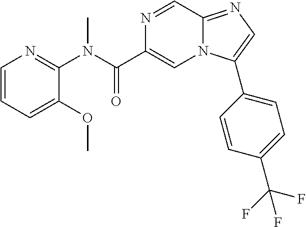 [SMILES: COc1cccnc1N(C)C(=O)c1cn2c(-c3ccc(C(F)(F)F)cc3)cnc2cn1]

The compound was synthesized using a procedure analogous to one use for synthesis of 72 (method II) using intermediate I-46. MS m/z 428.1 (M+H)+; r.t.=1.839.

### Synthesis of Compound 17

### 3-(4-chlorophenyl)-N-(4-methanesulfonylphenyl)-N-methylimidazo[1,2-a]pyrazine-6-carboxamide

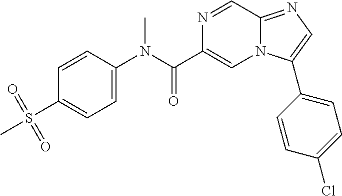 [SMILES: CN(C(=O)c1cn2c(-c3ccc(Cl)cc3)cnc2cn1)c1ccc(S(C)(=O)=O)cc1]

The compound was synthesized using I-66 and an appropriate boronic acid/ester using a procedure similar Suzuki coupling protocol described in 228. MS m/z 441.0 (M+H)+; r.t.=1.454.

### Synthesis of Compound 18

### N-{[3-(4-chlorophenyl)imidazo[1,2-a]pyrazin-6-yl]methyl}-4-fluoro-N-methylaniline

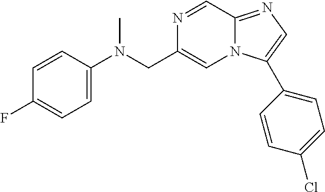 [SMILES: CN(Cc1cn2c(-c3ccc(Cl)cc3)cnc2cn1)c1ccc(F)cc1]

The compound was synthesized using I-62 and an appropriate boronic acid/ester using a procedure similar Suzuki coupling protocol described in 228. MS m/z 367.1 (M+H)+; r.t.=1.741.

### Synthesis of Compound 19

### N-(4-cyanophenyl)-N-methyl-3-(5-methylpyridin-2-yl)imidazo[1,2-a]pyrazine-6-carboxamide

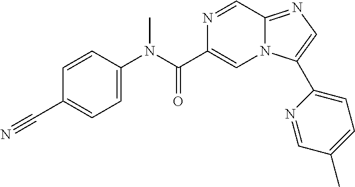 [SMILES: Cc1ccc(-c2cnc3cnc(C(=O)N(C)c4ccc(C#N)cc4)cn23)nc1]

The compound was synthesized using I-59 and an appropriate boronic acid/ester using a procedure similar Suzuki coupling protocol described in 228. M/Z=369.13 (M+1), r.t=1.31 mins.

### Synthesis of Compound 20

### N-(4-cyanophenyl)-3-(2,4-dichlorophenyl)-N-methylimidazo[1,2-a]pyrazine-6-carboxamide

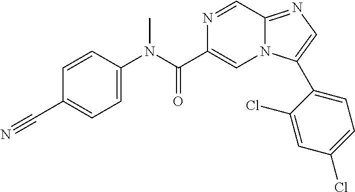 [SMILES: CN(C(=O)c1cn2c(-c3ccc(Cl)cc3Cl)cnc2cn1)c1ccc(C#N)cc1]

The compound was synthesized using I-62 and an appropriate boronic acid/ester using a procedure similar Suzuki coupling protocol described in 228. M/Z=422.1 (M+1), r.t=2.06 mins.

### Synthesis of Compound 21

### N-({3-[4-(difluoromethoxy)phenyl]imidazo[1,2-a]pyrazin-6-yl}methyl)-4-fluoro-N-methylaniline

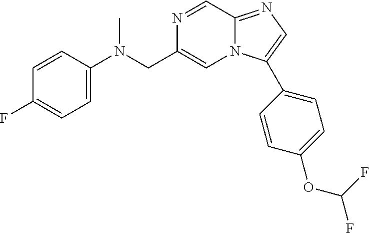 [SMILES: CN(Cc1cn2c(-c3ccc(OC(F)F)cc3)cnc2cn1)c1ccc(F)cc1]

The compound was synthesized using I-62 and an appropriate boronic acid/ester using a procedure similar Suzuki coupling protocol described in 228. MS m/z 399.1 (M+H)+; r.t.=1.712.

### Synthesis of Compound 22

### 3-(4-carbamoylphenyl)-N-(3,4-difluorophenyl)-N-methylimidazo[1,2-a]pyrazine-6-carboxamide

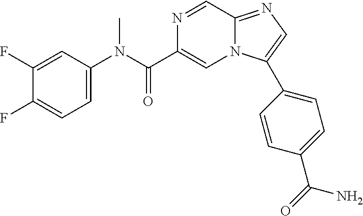 [SMILES: CN(C(=O)c1cn2c(-c3ccc(C(N)=O)cc3)cnc2cn1)c1ccc(F)c(F)c1]

The compound was synthesized using I-65 and an appropriate boronic acid/ester using a procedure similar Suzuki coupling protocol described in 228. MS m/z 408.1 (M+H)+; r.t.=1.248.

### Synthesis of Compound 23

### N-(2,4-difluorophenyl)-N-methyl-3-[4-(trifluoromethyl)phenyl]imidazo[1,2-a]pyrazine-6-carboxamide

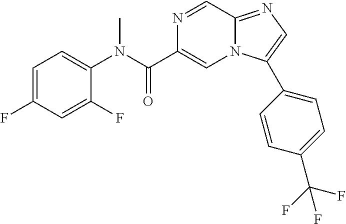 [SMILES: CN(C(=O)c1cn2c(-c3ccc(C(F)(F)F)cc3)cnc2cn1)c1ccc(F)cc1F]

The compound was synthesized using a procedure analogous to one use for synthesis of 72 (method II). MS m/z 433.1 (M+H)+; r.t.=1.929.

### Synthesis of Compound 24

### N-methyl-4-(morpholin-4-yl)-N-{3-[4-(trifluoromethyl)phenyl]imidazo[1,2-a]pyrazin-6-yl}benzamide

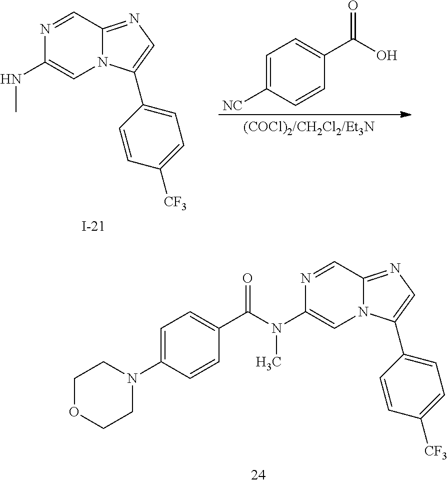

To a solution of acid in dichloromethane was added triethyl amine and a catalytic amount of DMF. The reaction mixture was cooled to 0° C. Oxalyl chloride was added slowly dripwise and the reaction mixture was stirred for 15 minutes. The solvent was removed. A solution of imidazolopyrazine in dichloromethane containing triethylamine was added to the acid chloride at 0° C. and the reaction mixture was stirred at room temperature for 2 hours. The reaction mixture was diluted with water and extracted with dichloromethane. The organic extracts were washed with brine and dried over MgSO₄ and concentrated. The crude reaction mixture was purified by flash chromatography (silica. 0-100% ethyl acetate/hexanes). ¹H NMR (400 MHz, CDCl3) δ 9.11 (s, 1H), 7.91 (s, 1H), 7.78 (s, 1H), 7.66 (d, J=8.1, 2H), 7.37 (d, J=8.8, 2H), 7.16 (d, J=8.0, 2H), 6.82 (d, J=8.8, 2H), 3.86-3.77 (m, 4H), 3.58 (s, 3H), 3.23-3.15 (m, 4H).

### Synthesis of Compound 25

### 4-[({3-[4-(5-amino-1,3,4-thiadiazol-2-yl)phenyl]imidazo[1,2-a]pyrazin-6-yl}methyl)(methyl)amino]benzonitrile

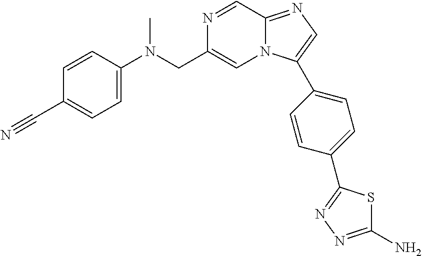 [SMILES: CN(Cc1cn2c(-c3ccc(-c4nnc(N)s4)cc3)cnc2cn1)c1ccc(C#N)cc1]

The compound was synthesized using I-15 and an appropriate boronic acid/ester using a procedure similar Suzuki coupling protocol described in 228. M/Z=439.1 (M+1), r.t=1.36 mins.

### Synthesis of Compound 26

### 3-(4-chloro-2-methylphenyl)-N-(4-cyanophenyl)-N-methylimidazo[1,2-a]pyrazine-6-carboxamide

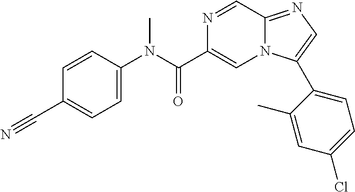 [SMILES: Cc1cc(Cl)ccc1-c1cnc2cnc(C(=O)N(C)c3ccc(C#N)cc3)cn12]

The compound was synthesized using I-59 and an appropriate boronic acid/ester using a procedure similar Suzuki coupling protocol described in 228. M/Z=402.2 (M+1), r.t=2.07 mins.

### Synthesis of Compound 27

### 3-(2-chloropyridin-4-yl)-N-(4-cyanophenyl)-N-methylimidazo[1,2-a]pyrazine-6-carboxamide

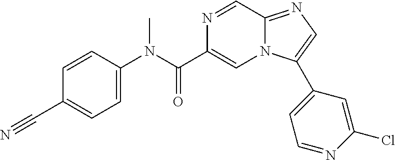 [SMILES: CN(C(=O)c1cn2c(-c3ccnc(Cl)c3)cnc2cn1)c1ccc(C#N)cc1]

The compound was synthesized using I-59 and an appropriate boronic acid/ester using a procedure similar Suzuki coupling protocol described in 228. MS m/z 389.1 (M+H)+; r.t.=1.630.

### Synthesis of Compound 28

### -({3-[4-(5-amino-1,3,4-thiadiazol-2-yl)phenyl]imidazo[1,2-a]pyrazin-6-yl}methyl)-6-chloro-N-methylpyridin-3-amine

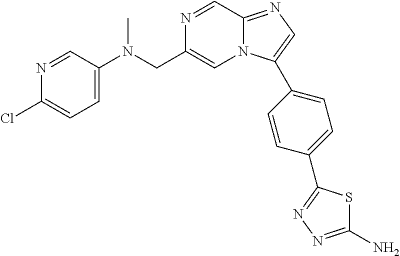 [SMILES: CN(Cc1cn2c(-c3ccc(-c4nnc(N)s4)cc3)cnc2cn1)c1ccc(Cl)nc1]

The compound was synthesized using I-76 and an appropriate boronic acid/ester using a procedure similar Suzuki coupling protocol described in 228. MS m/z 449.1 (M+H)+; r.t.=1.240.

### Synthesis of Compound 29

### N-(4-cyanophenyl)-3-(3,5-difluoropyridin-2-yl)-N-methylimidazo[1,2-a]pyrazine-6-carboxamide

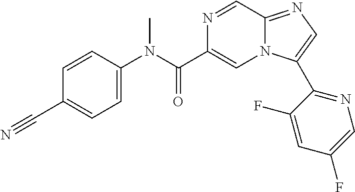 [SMILES: CN(C(=O)c1cn2c(-c3ncc(F)cc3F)cnc2cn1)c1ccc(C#N)cc1]

The compound was synthesized using I-59 and an appropriate boronic acid/ester using a procedure similar Suzuki coupling protocol described in 228. M/Z=391.10 (M+1), r.t=1.35 mins.

### Synthesis of Compound 30

### N-(4-cyanophenyl)-3-(4-methoxy-3-methylphenyl)-N-methylimidazo[1,2-a]pyrazine-6-carboxamide

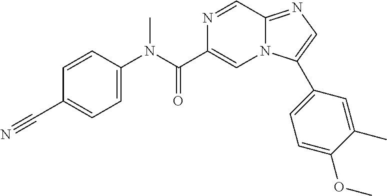 [SMILES: COc1ccc(-c2cnc3cnc(C(=O)N(C)c4ccc(C#N)cc4)cn23)cc1C]

The compound was synthesized using I-59 and an appropriate boronic acid/ester using a procedure similar Suzuki coupling protocol described in 228. M/Z=398.2 (M+1), r.t=2.01 mins.

### Synthesis of Compound 31

### 3-[2-chloro-4-(trifluoromethyl)phenyl]-N-(4-cyanophenyl)-N-methylimidazo[1,2-a]pyrazine-6-carboxamide

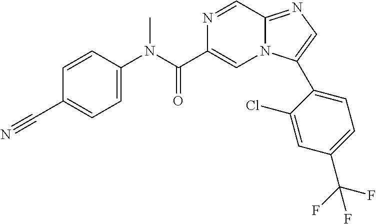 [SMILES: CN(C(=O)c1cn2c(-c3ccc(C(F)(F)F)cc3Cl)cnc2cn1)c1ccc(C#N)cc1]

The compound was synthesized using I-59 and an appropriate boronic acid/ester using a procedure similar Suzuki coupling protocol described in 228. M/Z=456.1 (M+1), r.t=2.23 mins.

### Synthesis of Compound 32

### N-(3-chloro-4-fluorophenyl)-N-methyl-3-[4-(trifluoromethyl)phenyl]imidazo[1,2-a]pyrazine-6-carboxamide

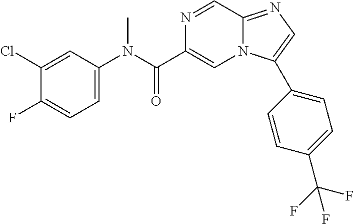 [SMILES: CN(C(=O)c1cn2c(-c3ccc(C(F)(F)F)cc3)cnc2cn1)c1ccc(F)c(Cl)c1]

The compound was synthesized using a procedure analogous to one use for synthesis of 72 (method II). MS m/z 449.1 (M+H)+; r.t.=2.320.

### Synthesis of Compound 33

### 3-acetyl-N-(6-chloropyridin-3-yl)-N-methylimidazo[1,2-a]pyrazine-6-carboxamide

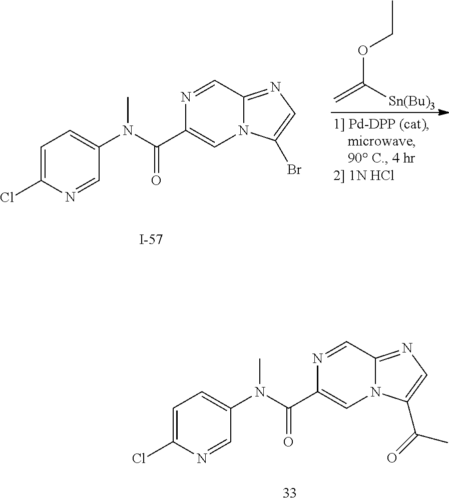

To the solution of 3-bromo-N-(4-chlorophenyl)-N-methylimidazo[1,2-a]pyrazine-6-carboxamide (0.06 g, 0.163 mmols) in THF was added tributyl(1-ethoxyvinyl)stannane (0.054 g, 0.149 mmol, 0.9 eq) followed by catalytic DPP-Pd. The reaction mixture was heated at 120° C. for 4 hours in a microwave. Subsequently, the reaction mixture was treated with 1 N HCl. The product was isolated via column chromatography. Yield=10 mg (0.03 mmol, 18%) of the desired product. M/Z=330.1 (M+1), r.t=1.08 mins.

### Synthesis of Compound 34

### 4-chloro-N-methyl-N-{[3-(1-methyl-1H-indazol-5-yl)imidazo[1,2-a]pyrazin-6-yl]methyl}aniline

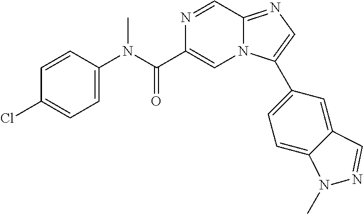 [SMILES: CN(C(=O)c1cn2c(-c3ccc4c(cnn4C)c3)cnc2cn1)c1ccc(Cl)cc1]

The compound was synthesized using I-63 and an appropriate boronic acid/ester using a procedure similar Suzuki coupling protocol described in 228.

### Synthesis of Compound 35

### N-(3-chlorophenyl)-N-methyl-3-[4-(trifluoromethyl)phenyl]imidazo[1,2-a]pyrazine-6-carboxamide

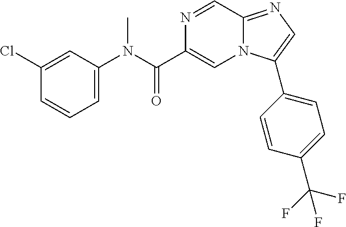 [SMILES: CN(C(=O)c1cn2c(-c3ccc(C(F)(F)F)cc3)cnc2cn1)c1cccc(Cl)c1]

The compound was synthesized using a procedure analogous to one use for synthesis of 72 (method II). MS m/z 431.1 (M+H)+; r.t.=2.240

### Synthesis of Compound 36

### 4-(6-{[(4-fluorophenyl)(methyl)amino]methyl}imidazo[1,2-a]pyrazin-3-yl)-N,N-dimethylaniline

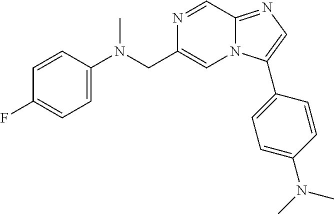 [SMILES: CN(C)c1ccc(-c2cnc3cnc(CN(C)c4ccc(F)cc4)cn23)cc1]

The compound was synthesized using I-62 and an appropriate boronic acid/ester using a procedure similar Suzuki coupling protocol described in 228. MS m/z 376.2 (M+H)+; r.t.=1.414.

### Synthesis of Compound 37

### 4-(6-{[(4-chlorophenyl)(methyl)amino]methyl}imidazo[1,2-a]pyrazin-3-yl)-N,N-dimethylaniline

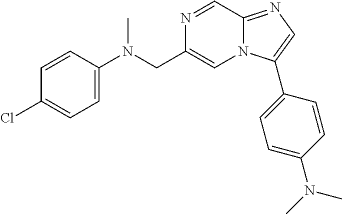 [SMILES: CN(C)c1ccc(-c2cnc3cnc(CN(C)c4ccc(Cl)cc4)cn23)cc1]

The compound was synthesized using I-63 and an appropriate boronic acid/ester using a procedure similar Suzuki coupling protocol described in 228. MS m/z 392.1 (M+H)+; r.t.=1.698.

### Synthesis of Compound 38

### N-(4-cyanophenyl)-N-methyl-3-[4-(methylcarbamoyl)phenyl]imidazo[1,2-a]pyrazine-6-carboxamide

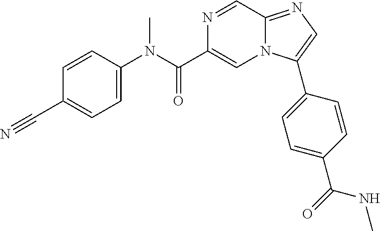 [SMILES: CNC(=O)c1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. 1H NMR (400 MHz, DMSO) δ 8.90 (d, J=1.4 Hz, 1H), 8.82 (d, J=1.3 Hz, 1H), 8.63 (d, J=4.6 Hz, 1H), 8.22 (s, 1H), 8.06 (d, J=8.4 Hz, 2H), 7.85 (d, J=8.4, 2H), 7.80-7.73 (m, 2H), 7.47 (d, J=8.6 Hz, 2H), 3.48 (s, 3H), 2.85 (d, J=4.5 Hz, 3H). M/Z=411.1 (M+1), r.t=1.11 mins.

### Synthesis of Compound 39

### N-(5-ethylpyridin-2-yl)-N-methyl-3-[4-(trifluoromethyl)phenyl]imidazo[1,2-a]pyrazine-6-carboxamide

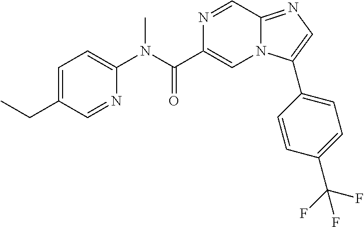 [SMILES: CCc1ccc(N(C)C(=O)c2cn3c(-c4ccc(C(F)(F)F)cc4)cnc3cn2)nc1]

The compound was synthesized using a procedure analogous to one use for synthesis of 72 (method II). ¹H NMR (400 MHz, MeOD) δ 8.90 (s, 1H), 8.67 (s, 1H), 8.11 (s, 2H), 7.98-7.88 (m, 4H), 7.67 (d, J=8.2 Hz, 1H), 7.26 (d, J=8.2 Hz, 1H), 3.56 (s, 3H), 2.65 (dd, J=7.6 Hz, 2H), 1.22 (t, J=7.6 Hz, 3H). ESI-MS m/z 426.14 [M+1]. RT: 1.68 min.

### Synthesis of Compound 40

### N-methyl-N-[2-(trifluoromethoxy)phenyl]-3-[4-(trifluoromethyl)phenyl]imidazo[1,2-a]pyrazine-6-carboxamide

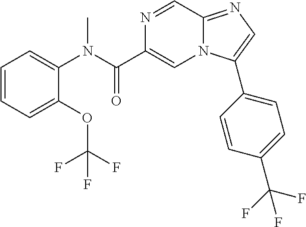 [SMILES: CN(C(=O)c1cn2c(-c3ccc(C(F)(F)F)cc3)cnc2cn1)c1ccccc1OC(F)(F)F]

The compound was synthesized using a procedure analogous to one use for synthesis of 72 (method II). MS m/z 481.0 (M+H)+; r.t.=2.380.

### Synthesis of Compound 41

### N-(2,4-dichlorophenyl)-3-(4-methoxyphenyl)-N-methylimidazo[1,2-a]pyrazine-6-carboxamide

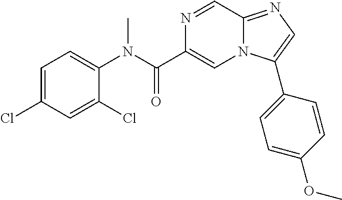 [SMILES: COc1ccc(-c2cnc3cnc(C(=O)N(C)c4ccc(Cl)cc4Cl)cn23)cc1]

The compound was synthesized using a procedure analogous to one use for synthesis of 72 (method II). MS m/z 427.1 (M+H)+; r.t.=2.220.

### Synthesis of Compound 42

### -(3-chloro-4-methoxyphenyl)-N-(4-cyanophenyl)-N-methylimidazo[1,2-a]pyrazine-6-carboxamide

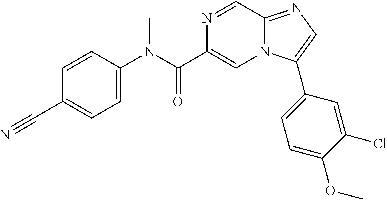 [SMILES: COc1ccc(-c2cnc3cnc(C(=O)N(C)c4ccc(C#N)cc4)cn23)cc1Cl]

The compound was synthesized using I-59 and an appropriate boronic acid/ester using a procedure similar Suzuki coupling protocol described in 228. M/Z=418.1 (M+1), r.t=1.92 mins.

### Synthesis of Compound 43

### N-methyl-N-(4-phenoxyphenyl)-3-[4-(trifluoromethyl)phenyl]imidazo[1,2-a]pyrazine-6-carboxamide

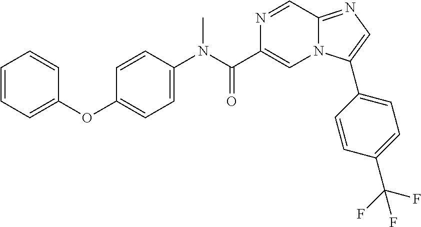 [SMILES: CN(C(=O)c1cn2c(-c3ccc(C(F)(F)F)cc3)cnc2cn1)c1ccc(Oc2ccccc2)cc1]

The compound was synthesized using a procedure analogous to one use for synthesis of 72 (method II). MS m/z 489.1 (M+H)+; r.t.=2.420.

### Synthesis of Compound 44

### 4-chloro-N-{[3-(4-chlorophenyl)imidazo[1,2-a]pyrazin-6-yl]methyl}-N-methylaniline

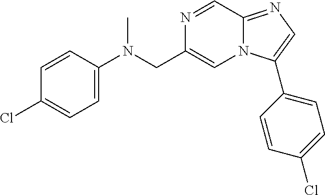 [SMILES: CN(Cc1cn2c(-c3ccc(Cl)cc3)cnc2cn1)c1ccc(Cl)cc1]

The compound was synthesized using I-63 and an appropriate boronic acid/ester using a procedure similar Suzuki coupling protocol described in 228. MS m/z 383.0 (M+H)+; r.t.=2.014.

### Synthesis of Compound 45

### N,5-dimethyl-N-{[3-(1-methyl-1H-indazol-5-yl)imidazo[1,2-a]pyrazin-6-yl]methyl}pyridin-2-amine

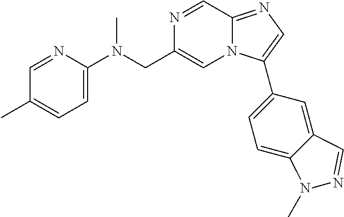 [SMILES: Cc1ccc(N(C)Cc2cn3c(-c4ccc5c(cnn5C)c4)cnc3cn2)nc1]

The compound was synthesized using I-77 and an appropriate boronic acid/ester using a procedure similar Suzuki coupling protocol described in 228. MS m/z 384.2 (M+H)+; r.t.=1.092.

### Synthesis of Compound 46

### N-(4-cyanophenyl)-N-methyl-3-[3-(1H-pyrazol-4-yl)phenyl]imidazo[1,2-a]pyrazine-6-carboxamide

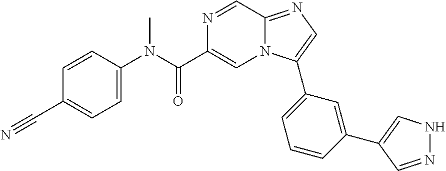 [SMILES: CN(C(=O)c1cn2c(-c3cccc(-c4cn[nH]c4)c3)cnc2cn1)c1ccc(C#N)cc1]

The compound was synthesized using I-59 and an appropriate boronic acid/ester using a procedure similar Suzuki coupling protocol described in 228. M/Z=420.1 (M+1), r.t=1.83 mins.

### Synthesis of Compound 47

### N,5-dimethyl-N-({3-[4-(1H-pyrazol-5-yl)phenyl]imidazo[1,2-a]pyrazin-6-yl}methyl)pyridin-2-amine

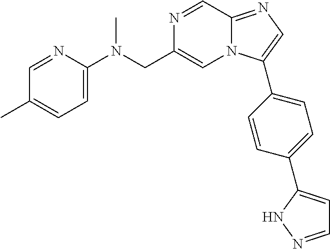 [SMILES: Cc1ccc(N(C)Cc2cn3c(-c4ccc(-c5ccn[nH]5)cc4)cnc3cn2)nc1]

The compound was synthesized using I-77 and an appropriate boronic acid/ester using a procedure similar Suzuki coupling protocol described in 228. MS m/z 396.2 (M+H)+; r.t.=1.112.

### Synthesis of Compound 48

### N-(4-cyanophenyl)-3-(3-methoxyphenyl)-N-methylimidazo[1,2-a]pyrazine-6-carboxamide

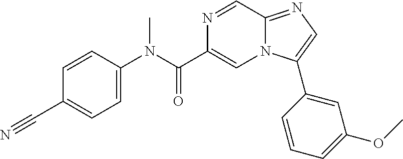 [SMILES: COc1cccc(-c2cnc3cnc(C(=O)N(C)c4ccc(C#N)cc4)cn23)c1]

The compound was synthesized using I-59 and an appropriate boronic acid/ester using a procedure similar Suzuki coupling protocol described in 228. M/Z=384.1 (M+1), r.t=1.59 mins.

### Synthesis of Compound 49

### N-(2,4-dichlorophenyl)-N-methyl-3-[4-(trifluoromethyl)phenyl]imidazo[1,2-a]pyrazine-6-carboxamide

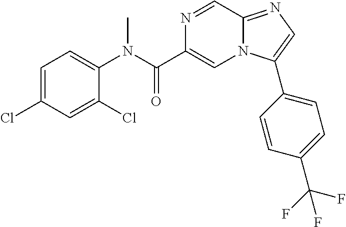 [SMILES: CN(C(=O)c1cn2c(-c3ccc(C(F)(F)F)cc3)cnc2cn1)c1ccc(Cl)cc1Cl]

The compound was synthesized using a procedure analogous to one use for synthesis of 72 (method II). MS m/z 465.0 (M+H)+; r.t.=2.500.

### Synthesis of Compound 50

### N-[4-(difluoromethoxy)phenyl]-N-methyl-3-[4-(trifluoromethyl)phenyl]imidazo[1,2-a]pyrazine-6-carboxamide

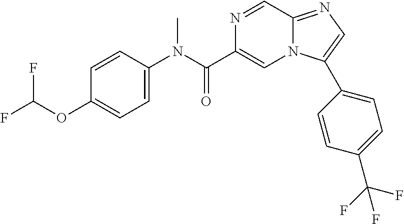 [SMILES: CN(C(=O)c1cn2c(-c3ccc(C(F)(F)F)cc3)cnc2cn1)c1ccc(OC(F)F)cc1]

The compound was synthesized using a procedure analogous to one use for synthesis of 72 (method II). MS m/z 463.1 (M+H)+; r.t.=1.771.

### Synthesis of Compound 51

### N-methyl-N-[4-(1-methyl-1H-pyrazol-4-yl)phenyl]-3-[4-(trifluoromethyl)phenyl]imidazo[1,2-a]pyrazine-6-carboxamide

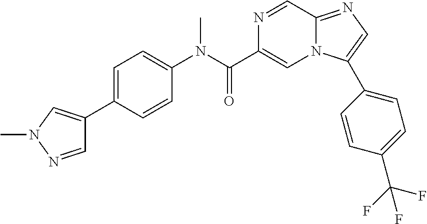 [SMILES: CN(C(=O)c1cn2c(-c3ccc(C(F)(F)F)cc3)cnc2cn1)c1ccc(-c2cnn(C)c2)cc1]

The compound was synthesized using a procedure analogous to one use for synthesis of 72 (method II). using intermediate I-34. MS m/z 477.1 (M+H)+; r.t.=1.546.

### Synthesis of Compound 52

### N-(3-bromo-4-fluorophenyl)-N-methyl-3-[4-(trifluoromethyl)phenyl]imidazo[1,2-a]pyrazine-6-carboxamide

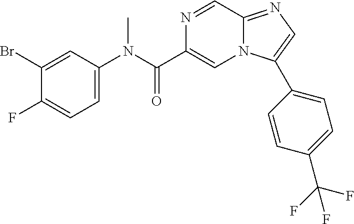 [SMILES: CN(C(=O)c1cn2c(-c3ccc(C(F)(F)F)cc3)cnc2cn1)c1ccc(F)c(Br)c1]

The compound was synthesized using a procedure analogous to one use for synthesis of 72 (method II). MS m/z 495.0 (M+H)+; r.t.=2.320.

### Synthesis of Compound 53

### N-(3,4-difluorophenyl)-N-methyl-3-(1-methyl-1H-indazol-5-yl)imidazo[1,2-a]pyrazine-6-carboxamide

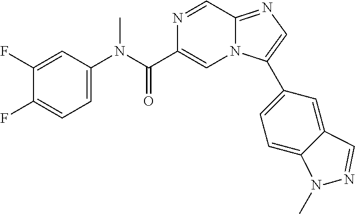 [SMILES: CN(C(=O)c1cn2c(-c3ccc4c(cnn4C)c3)cnc2cn1)c1ccc(F)c(F)c1]

The compound was synthesized using I-65 and an appropriate boronic acid/ester using a procedure similar Suzuki coupling protocol described in 228. ¹H NMR (400 MHz, DMSO) δ 8.76 (s, 1H), 8.75 (s, 1H), 8.14 (s, 1H), 8.07 (s, 1H), 8.05 (s, 1H), 7.84 (d, J=8.7, 1H), 7.65 (dd, J=1.6, 8.7, 1H), 7.48 (ddd, J=2.5, 7.3, 11.8, 1H), 7.27 (dd, J=9.1, 19.4, 1H), 7.05 (d, J=15.3, 1H), 4.07 (s, 3H), 3.34 (s, 3H); MS m/z 419.1 (M+H)+; r.t.=1.461.

### Synthesis of Compound 54

### N-(2-fluoropyridin-3-yl)-N-methyl-3-[4-(trifluoromethyl)phenyl]imidazo[1,2-a]pyrazine-6-carboxamide

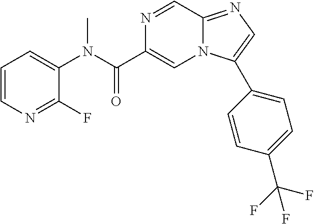 [SMILES: CN(C(=O)c1cn2c(-c3ccc(C(F)(F)F)cc3)cnc2cn1)c1cccnc1F]

The compound was synthesized using a procedure analogous to one use for synthesis of 72 (method II), using intermediate I-47. MS m/z 396.1 (M+H)+; r.t.=1.475.

### Synthesis of Compound 55

### N-(3,4-dichlorophenyl)-N-methyl-3-[4-(trifluoromethyl)phenyl]imidazo[1,2-a]pyrazine-6-carboxamide

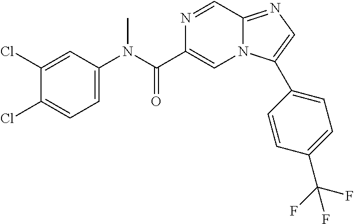 [SMILES: CN(C(=O)c1cn2c(-c3ccc(C(F)(F)F)cc3)cnc2cn1)c1ccc(Cl)c(Cl)c1]

The compound was synthesized using a procedure analogous to one use for synthesis of 72 (method II). MS m/z 465.0 (M+H)+; r.t.=1.938.

### Synthesis of Compound 56

### N-(4-chloro-3-methylphenyl)-N-methyl-3-[4-(trifluoromethyl)phenyl]imidazo[1,2-a]pyrazine-6-carboxamide

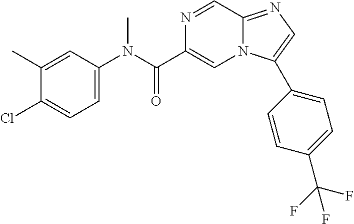 [SMILES: Cc1cc(N(C)C(=O)c2cn3c(-c4ccc(C(F)(F)F)cc4)cnc3cn2)ccc1Cl]

The compound was synthesized using a procedure analogous to one use for synthesis of 72 (method II). MS m/z 445.1 (M+H)+; r.t.=2.360.

### Synthesis of Compound 57

### N-(4-cyanophenyl)-3-[4-(difluoromethyl)phenyl]-N-methylimidazo[1,2-a]pyrazine-6-carboxamide

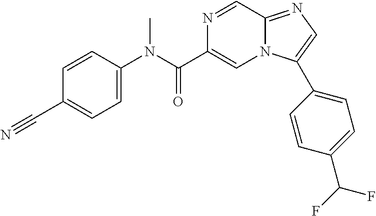 [SMILES: CN(C(=O)c1cn2c(-c3ccc(C(F)F)cc3)cnc2cn1)c1ccc(C#N)cc1]

The compound was synthesized using I-59 and an appropriate boronic acid/ester using a procedure similar Suzuki coupling protocol described in 228. M/Z=404.1 (M+1), r.t=1.59 mins

### Synthesis of Compound 58

### N-(6-bromopyridin-3-yl)-N-methyl-3-[4-(trifluoromethyl)phenyl]imidazo[1,2-a]pyrazine-6-carboxamide

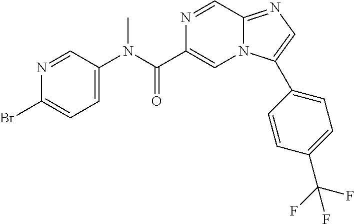 [SMILES: CN(C(=O)c1cn2c(-c3ccc(C(F)(F)F)cc3)cnc2cn1)c1ccc(Br)nc1]

The compound was synthesized using a procedure analogous to one use for synthesis of 72 (method II), using intermediate I-44. MS m/z 476.0 (M+H)+; r.t.=1.702.

### Synthesis of Compound 59

### N-(4-chloro-2-methoxyphenyl)-N-methyl-3-[4-(trifluoromethyl)phenyl]imidazo[1,2-a]pyrazine-6-carboxamide

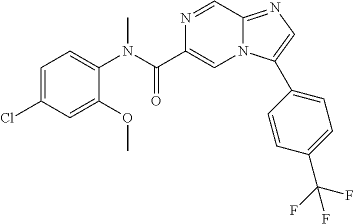 [SMILES: COc1cc(Cl)ccc1N(C)C(=O)c1cn2c(-c3ccc(C(F)(F)F)cc3)cnc2cn1]

The compound was synthesized using a procedure analogous to one use for synthesis of 72 (method II). MS m/z 461.0 (M+H)+; r.t.=1.809.

### Synthesis of Compound 60

### 3-[4-chloro-3-(trifluoromethyl)phenyl]-N-(4-cyanophenyl)-N-methylimidazo[1,2-a]pyrazine-6-carboxamide

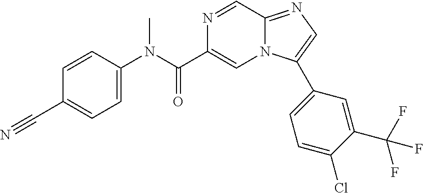 [SMILES: CN(C(=O)c1cn2c(-c3ccc(Cl)c(C(F)(F)F)c3)cnc2cn1)c1ccc(C#N)cc1]

The compound was synthesized using I-59 and an appropriate boronic acid/ester using a procedure similar Suzuki coupling protocol described in 228. M/Z=456.1 (M+1), r.t=2.18 mins.

### Synthesis of Compound 61

### N-(2,4-dichlorophenyl)-N-methyl-3-(4-methylphenyl)imidazo[1,2-a]pyrazine-6-carboxamide

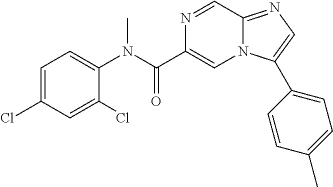 [SMILES: Cc1ccc(-c2cnc3cnc(C(=O)N(C)c4ccc(Cl)cc4Cl)cn23)cc1]

The compound was synthesized using a procedure analogous to one use for synthesis of 72 (method II). MS m/z 411.1 (M+H)+; r.t.=2.370.

### Synthesis of Compound 62

### 4-[methyl({[3-(1-methyl-1H-indazol-5-yl)imidazo[1,2-a]pyrazin-6-yl]methyl})amino]benzonitrile

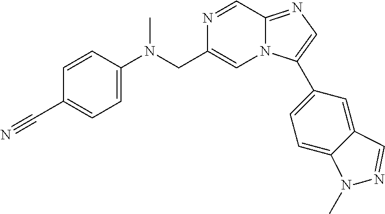 [SMILES: CN(Cc1cn2c(-c3ccc4c(cnn4C)c3)cnc2cn1)c1ccc(C#N)cc1]

The compound was synthesized using I-63 and an appropriate boronic acid/ester using a procedure similar Suzuki coupling protocol described in 228. MS m/z 394.1 (M+H)+; r.t.=1.472.

### Synthesis of Compound 63

### N-(5-fluoropyridin-2-yl)-N-methyl-3-[4-(trifluoromethyl)phenyl]imidazo[1,2-a]pyrazine-6-carboxamide

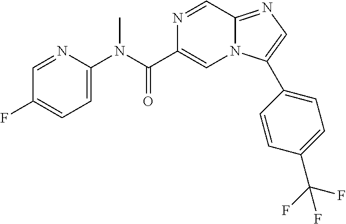 [SMILES: CN(C(=O)c1cn2c(-c3ccc(C(F)(F)F)cc3)cnc2cn1)c1ccc(F)cn1]

The compound was synthesized using a procedure analogous to one use for synthesis of 72 (method II); using intermediate I-38. MS m/z 416.0 (M+H)+; r.t.=1.910.

### Synthesis of Compound 64

### 4-chloro-N-({3-[4-(difluoromethoxy)phenyl]imidazo[1,2-a]pyrazin-6-yl}methyl)-N-methylaniline

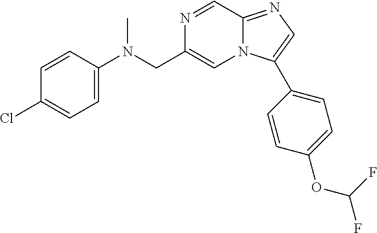 [SMILES: CN(Cc1cn2c(-c3ccc(OC(F)F)cc3)cnc2cn1)c1ccc(Cl)cc1]

The compound was synthesized using I-63 and an appropriate boronic acid/ester using a procedure similar Suzuki coupling protocol described in 228. MS m/z 415.1 (M+H)+; r.t.=1.929

### Synthesis of Compound 65

### N-(4-cyanophenyl)-3-(2-fluoro-4-methoxyphenyl)-N-methylimidazo[1,2-a]pyrazine-6-carboxamide

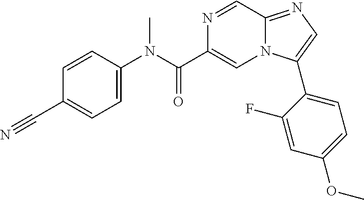 [SMILES: COc1ccc(-c2cnc3cnc(C(=O)N(C)c4ccc(C#N)cc4)cn23)c(F)c1]

The compound was synthesized using I-59 and an appropriate boronic acid/ester using a procedure similar Suzuki coupling protocol described in 228. M/Z=402.2 (M+1), r.t=1.86 mins.

### Synthesis of Compound 66

### N-(5-fluoropyridin-3-yl)-N-methyl-3-[4-(trifluoromethyl)phenyl]imidazo[1,2-a]pyrazine-6-carboxamide

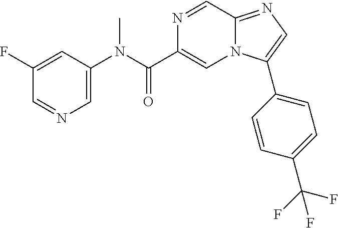 [SMILES: CN(C(=O)c1cn2c(-c3ccc(C(F)(F)F)cc3)cnc2cn1)c1cncc(F)c1]

The compound was synthesized using a procedure analogous to one use for synthesis of 72 (method II) using intermediate I-51. MS m/z 416.1 (M+H)+; r.t.=1.564.

### Synthesis of Compound 67

### N-(4-cyanophenyl)-3-(4-methoxyphenyl)-N-methylimidazo[1,2-a]pyrazine-6-carboxamide

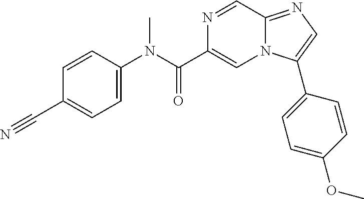 [SMILES: COc1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. MS m/z 384.2 (M+H)+; r.t.=1.860.

### Synthesis of Compound 68

### N-methyl-N-[4-(2-methyl-2H-1,2,3,4-tetrazol-5-yl)phenyl]-3-[4-(trifluoromethyl)phenyl]imidazo[1,2-a]pyrazine-6-carboxamide

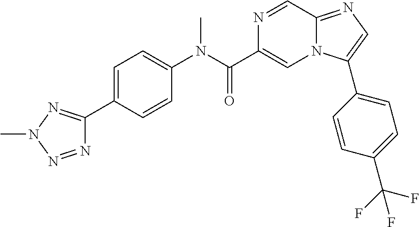 [SMILES: CN(C(=O)c1cn2c(-c3ccc(C(F)(F)F)cc3)cnc2cn1)c1ccc(-c2nnn(C)n2)cc1]

The compound was synthesized using a procedure analogous to one use for synthesis of 72 (method II). MS m/z 479.1 (M+H)+; r.t.=1.656.

### Synthesis of Compound 69

### N-(4-fluorophenyl)-3-(4-methoxyphenyl)-N-methylimidazo[1,2-a]pyrazine-6-carboxamide

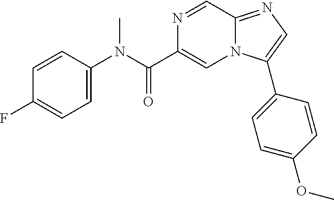 [SMILES: COc1ccc(-c2cnc3cnc(C(=O)N(C)c4ccc(F)cc4)cn23)cc1]

The compound was synthesized using I-64 and an appropriate boronic acid/ester using a procedure similar Suzuki coupling protocol described in 228. MS m/z 377.2 (M+H)+; r.t.=1.850.

### Synthesis of Compound 70

### 3-(4-chlorophenyl)-N-(2,4-dichlorophenyl)-N-methylimidazo[1,2-a]pyrazine-6-carboxamide

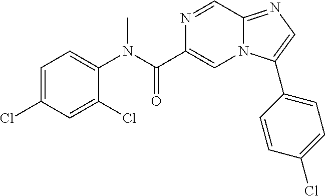 [SMILES: CN(C(=O)c1cn2c(-c3ccc(Cl)cc3)cnc2cn1)c1ccc(Cl)cc1Cl]

The compound was synthesized using a procedure analogous to one use for synthesis of 72 (method II). MS m/z 432.9 (M+H)+; r.t.=2.370.

### Synthesis of Compound 71

### 3-(4-chloro-3-fluorophenyl)-N-(4-cyanophenyl)-N-methylimidazo[1,2-a]pyrazine-6-carboxamide

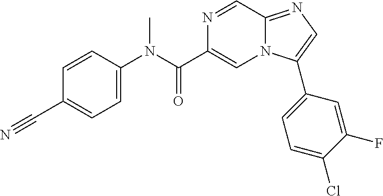 [SMILES: CN(C(=O)c1cn2c(-c3ccc(Cl)c(F)c3)cnc2cn1)c1ccc(C#N)cc1]

The compound was synthesized using I-59 and an appropriate boronic acid/ester using a procedure similar Suzuki coupling protocol described in 228. M/Z=406.1 (M+1), r.t=2.06 mins.

### Synthesis of Compound 72

### N-(4-cyanophenyl)-N-[2-(dimethylamino)ethyl]-3-[4-(trifluoromethyl)phenyl]imidazo[1,2-a]pyrazine-6-carboxamide

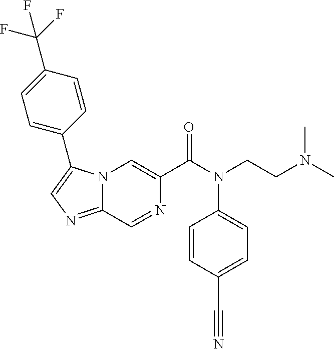 [SMILES: CN(C)CCN(C(=O)c1cn2c(-c3ccc(C(F)(F)F)cc3)cnc2cn1)c1ccc(C#N)cc1]

The compound was synthesized using a procedure analogous to one use for synthesis of 72 (method II) using intermediate I-32. MS m/z 479.2 (M+H)+; r.t.=1.450.

### Synthesis of Compound 73

### N-(2-chlorophenyl)-N-methyl-3-[4-(trifluoromethyl)phenyl]imidazo[1,2-a]pyrazine-6-carboxamide

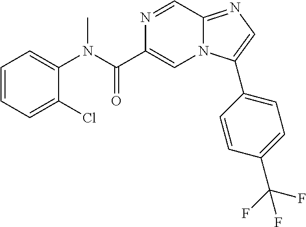 [SMILES: CN(C(=O)c1cn2c(-c3ccc(C(F)(F)F)cc3)cnc2cn1)c1ccccc1Cl]

The compound was synthesized using a procedure analogous to one use for synthesis of 72 (method II). MS m/z 431.1 (M+H)+; r.t.=2.270.

### Synthesis of Compound 74

### 3-[4-(5-amino-1,3,4-thiadiazol-2-yl)phenyl]-N-(4-cyanophenyl)-N-methylimidazo[1,2-a]pyrazine-6-carboxamide

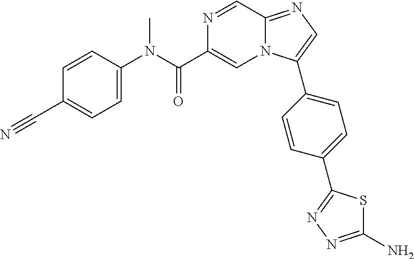 [SMILES: CN(C(=O)c1cn2c(-c3ccc(-c4nnc(N)s4)cc3)cnc2cn1)c1ccc(C#N)cc1]

The compound was synthesized using I-59 and an appropriate boronic acid/ester using a procedure similar Suzuki coupling protocol described in 228. 1H NMR (400 MHz, DMSO) δ 9.25 (s, 1H), 8.91 (dd, J=1.3, 3.7, 1H), 8.82 (d, J=1.3, 1H), 8.21 (d, J=2.8 Hz, 1H), 8.01 (m, 2H), 7.83 (m, 2H), 7.77 (d, J=8.6 Hz, 2H), 7.48 (dd, J=2.7, 8.6 Hz, 2H), 3.48 (s, 3H). M/Z=453.1 (M+1), rt=1.03 mins.

### Synthesis of Compound 75

### N-(4-cyanophenyl)-3-[4-(1,1-difluoroethyl)phenyl]-N-methylimidazo[1,2-a]pyrazine-6-carboxamide

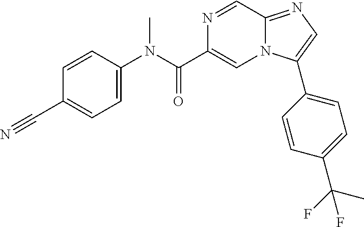 [SMILES: CN(C(=O)c1cn2c(-c3ccc(C(C)(F)F)cc3)cnc2cn1)c1ccc(C#N)cc1]

The compound was synthesized using I-59 and I-25 using a procedure similar Suzuki coupling protocol described in 228. M/Z=418.1 (M+1), r.t=1.60 mins.

### Synthesis of Compound 76

### N-(4-cyanophenyl)-N-methyl-3-[4-(trifluoromethyl)phenyl]imidazo[1,2-a]pyrazine-6-carboxamide

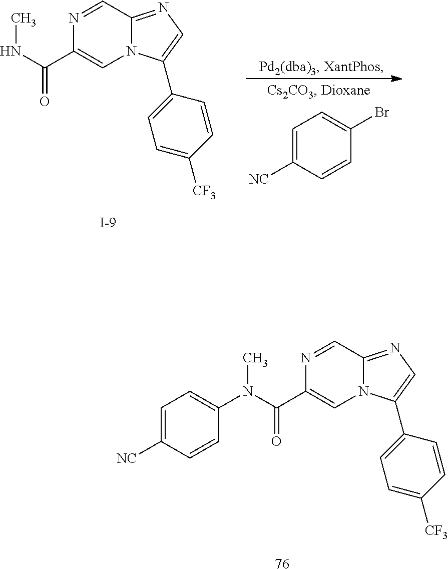

Pd₂(dba)₃ (1 mg, 0.00078 mmol) was added to a mixture of N-methyl-3-(4-(trifluoromethyl)phenyl)imidazo[1,2-a]pyrazine-6-carboxamide (25 mg, 0.078 mmol), 4-bromobenzonitrile (12 mg, 0.065 mmol), cesium carbonate (36 mg, 0.11 mmol), XantPhos (1.5 mg, 0.0023 mmol) and dioxane (0.25 mL). The reaction vessel was evacuated to degas the mixture. The reaction vessel was left under vacuum, sealed, and heated to 110° C. for 6 hours. After cooling to room temperature, the solvent was removed. The crude material was taken up in dichloromethane and filtered to remove any salts. The crude reaction mixture was purified by preparatory TLC to give the desired product. ¹H NMR (400 MHz, MeOD) δ 8.92 (d, J=1.4, 1H), 8.75 (s, 1H), 8.11 (s, 1H), 7.93 (d, J=8.5, 2H), 7.89 (d, J=8.5, 2H), 7.71-7.63 (m, 2H), 7.45-7.38 (m, 2H), 3.55 (s, 3H).

### Method II:

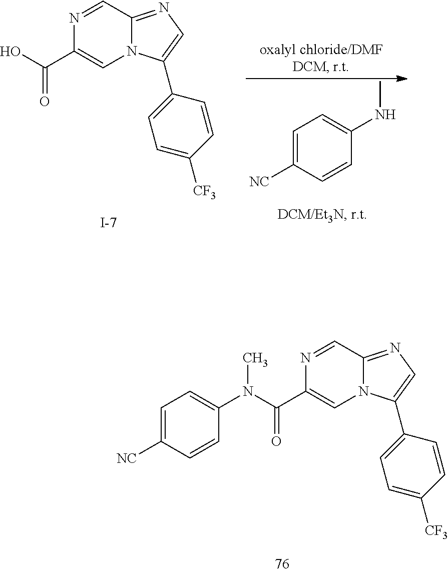

Oxalyl chloride (0.47 mL, 5.40 mmol) was added drop wise to a solution of 3-(4-(trifluoromethyl)phenyl)imidazo[1,2-a]pyrazine-6-carboxylic acid (826 mg, 2.70 mmol), DMF (0.05 mL) and dry dichloromethane (20 mL) at room temperature (gas evolution). After 30 minutes at room temperature, the solvent was removed in vacuo. The crude acid chloride was dissolved in dry dichloromethane (15 mL) and a solution of 4-(methylamino)benzonitrile (533 mg, 4.04 mmol in 5.0 mL of dichloromethane) was added drop wise at room temperature. Triethylamine (1.13 mL, 8.10 mmol) was added and the reaction stirred at room temperature for 3 hours. The solvent was removed in vacuo and the crude material was purified by flash chromatography (silica, 10-100% ethyl acetate/hexanes). The product was further recrystallized from methanol to give pure N-(4-cyanophenyl)-N-methyl-3-(4-(trifluoromethyl)phenyl)imidazo[1,2-a]pyrazine-6-carboxamide as white needles. ¹H NMR (400 MHz, DMSO) δ 9.72 (d, J=1.4, 1H), 9.65 (d, J=1.3, 1H), 9.06 (s, 1H), 8.80 (s, 4H), 8.57 (d, J=8.7, 2H), 8.27 (d, J=8.6, 2H), 4.28 (s, 3H).

### Method III:

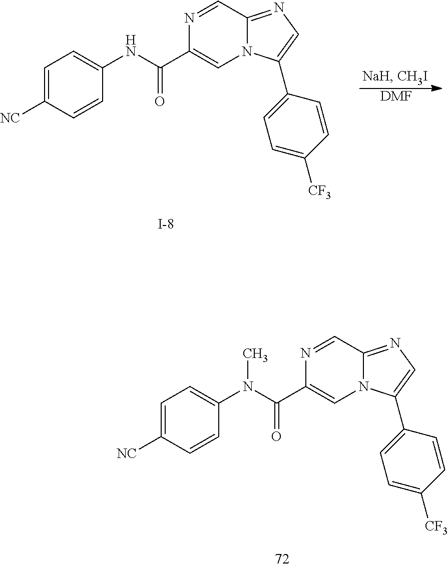

Sodium hydride (4.0 mg, 0.0921 mmol) was added to a solution of N-(4-cyanophenyl)-3-(4-(trifluoromethyl)phenyl)imidazo[1,2-a]pyrazine-6-carboxamide (25 mg, 0.0614 mmol) and DMF (0.70 mL) at room temperature. After 30 minutes, methyl iodide (0.006 mL, 0.0921 mL) was added. The reaction stirred at room temperature for 12 hours. After quenching with water, the solvent was removed in vacuo. The crude reaction mixture was purified by preparative HPLC (10-90% acetonitrile/water) to give the trifluoroacetamide salt of N-(4-cyanophenyl)-N-methyl-3-(4-(trifluoromethyl)phenyl)imidazo[1,2-a]pyrazine-6-carboxamide as a white colored solid. ¹H NMR (400 MHz, MeOD) δ 8.92 (d, J=1.4, 1H), 8.75 (s, 1H), 8.11 (s, 1H), 7.93 (d, J=8.5, 2H), 7.89 (d, J=8.5, 2H), 7.71-7.63 (m, 2H), 7.45-7.38 (m, 2H), 3.55 (s, 3H); MS m/z 422.0 (M+H)+; r.t.=1.930.

### Synthesis of Compound 77

### N-(4-cyanophenyl)-3-[4-(dimethylamino)phenyl]-N-methylimidazo[1,2-a]pyrazine-6-carboxamide

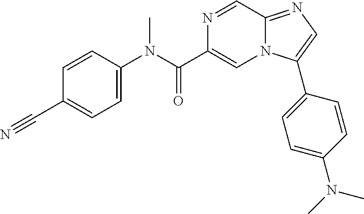 [SMILES: CN(C)c1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. 1H NMR (400 MHz, DMSO) δ 8.67 (dd, J=1.4, 11.4 Hz, 3H), 7.90 (s, 2H), 7.69 (m, 3H), 7.41 (dd, J=8.7, 18.4 Hz, 5H), 6.85 (d, J=8.9 Hz, 3H), 3.40 (s, 3H), 2.94 (s, 6H). M/Z=397.2 (M+1), r.t=1.21 mins.

### Synthesis of Compound 78

### N-(6-chloropyridin-3-yl)-N-methyl-3-[4-(trifluoromethyl)phenyl]imidazo[1,2-a]pyrazine-6-carboxamide

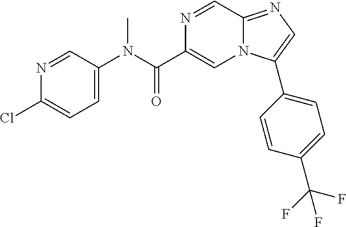 [SMILES: CN(C(=O)c1cn2c(-c3ccc(C(F)(F)F)cc3)cnc2cn1)c1ccc(Cl)nc1]

The compound was synthesized using I-57 and an appropriate boronic acid/ester using a procedure similar Suzuki coupling protocol described in 228. ¹H NMR (400 MHz, DMSO) δ 8.92 (d, J=1.4, 1H), 8.85 (s, 1H), 8.30 (s, 1H), 8.26 (s, 1H), 8.05-7.93 (m, 4H), 7.87 (dd, J=2.8, 8.5, 1H), 7.49 (d, J=8.5, 1H), 3.45 (s, 3H); MS m/z 432.1 (M+H)+; r.t.=2.070.

### Synthesis of Compound 79

### N-(6-fluoropyridin-3-yl)-N-methyl-3-[4-(trifluoromethyl)phenyl]imidazo[1,2-a]pyrazine-6-carboxamide

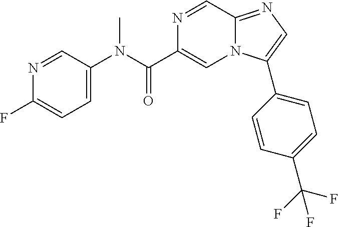 [SMILES: CN(C(=O)c1cn2c(-c3ccc(C(F)(F)F)cc3)cnc2cn1)c1ccc(F)nc1]

The compound was synthesized using a procedure analogous to one use for synthesis of 72 (method II) using I-52. MS m/z 416.1 (M+H)+; r.t.=2.000.

### Synthesis of Compound 80

### -(6-chloropyridin-3-yl)-N-methyl-3-(4-methylphenyl)imidazo[1,2-a]pyrazine-6-carboxamide

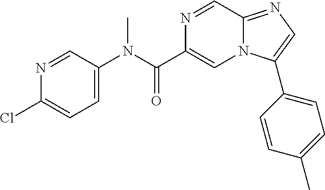 [SMILES: Cc1ccc(-c2cnc3cnc(C(=O)N(C)c4ccc(Cl)nc4)cn23)cc1]

The compound was synthesized using I-57 and an appropriate boronic acid/ester using a procedure similar Suzuki coupling protocol described in 228. MS m/z 378.1 (M+H)+; r.t.=1.940.

### Synthesis of Compound 81

### -(4-chlorophenyl)-N-(6-chloropyridin-3-yl)-N-methylimidazo[1,2-a]pyrazine-6-carboxamide

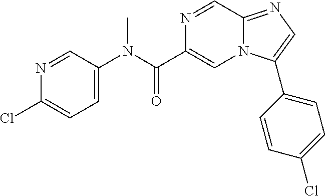 [SMILES: CN(C(=O)c1cn2c(-c3ccc(Cl)cc3)cnc2cn1)c1ccc(Cl)nc1]

The compound was synthesized using I-57 and an appropriate boronic acid/ester using a procedure similar Suzuki coupling protocol described in 228. MS m/z 398.1 (M+H)+; r.t.=1.960.

### Synthesis of Compound 82

### N-(4-cyanophenyl)-N-methyl-3-[6-(pyrrolidin-1-yl)pyridin-3-yl]imidazo[1,2-a]pyrazine-6-carboxamide

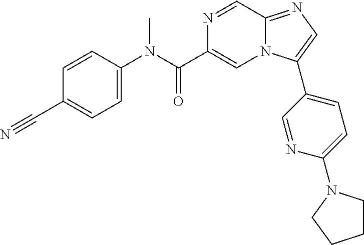 [SMILES: CN(C(=O)c1cn2c(-c3ccc(N4CCCC4)nc3)cnc2cn1)c1ccc(C#N)cc1]

The compound was synthesized using I-59 and an appropriate boronic acid/ester using a procedure similar Suzuki coupling protocol described in 228. M/Z=424.1 (M+1), r.t=1.3 mins.

### Synthesis of Compound 83

### N-(5-cyanopyridin-2-yl)-N-methyl-3-(1-methyl-1H-indazol-5-yl)imidazo[1,2-a]pyrazine-6-carboxamide

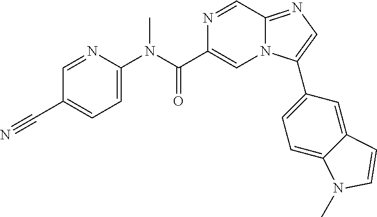 [SMILES: CN(C(=O)c1cn2c(-c3ccc4c(ccn4C)c3)cnc2cn1)c1ccc(C#N)cn1]

The compound was synthesized using I-75 and an appropriate boronic acid/ester using a procedure similar Suzuki coupling protocol described in 228. M/Z=409.1 (M+1), r.t=1.28 mins.

### Synthesis of Compound 84

### N-(6-chloropyridin-3-yl)-N-methyl-3-(1-methyl-1H-indol-5-yl)imidazo[1,2-a]pyrazine-6-carboxamide

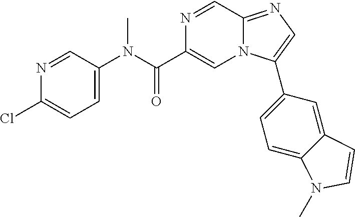 [SMILES: CN(C(=O)c1cn2c(-c3ccc4c(ccn4C)c3)cnc2cn1)c1ccc(Cl)nc1]

The compound was synthesized using I-57 and an appropriate boronic acid/ester using a procedure similar Suzuki coupling protocol described in 228. MS m/z 417.1 (M+H)+; r.t.=1.94.

### Synthesis of Compound 85

### N-(5-cyanopyridin-2-yl)-3-[4-(difluoromethoxy)phenyl]-N-methylimidazo[1,2-a]pyrazine-6-carboxamide

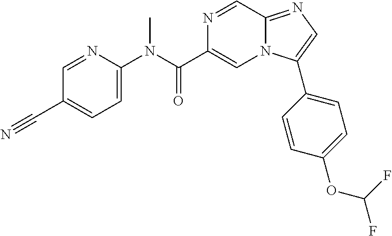 [SMILES: CN(C(=O)c1cn2c(-c3ccc(OC(F)F)cc3)cnc2cn1)c1ccc(C#N)cn1]

The compound was synthesized using I-75 and an appropriate boronic acid/ester using a procedure similar Suzuki coupling protocol described in 228. M/Z=421.1 (M+1), r.t=1.44 mins.

### Synthesis of Compound 86

### N-(6-fluoropyridin-2-yl)-N-methyl-3-[4-(trifluoromethyl)phenyl]imidazo[1,2-a]pyrazine-6-carboxamide

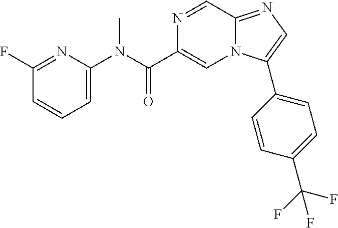 [SMILES: CN(C(=O)c1cn2c(-c3ccc(C(F)(F)F)cc3)cnc2cn1)c1cccc(F)n1]

The compound was synthesized using a procedure analogous to one use for synthesis of 72 (method II) using I-39. MS m/z 416.0 (M+H)+; r.t.=1.722.

### Synthesis of Compound 87

### N-(4-cyanophenyl)-N-methyl-3-[4-(1H-pyrazol-1-yl)phenyl]imidazo[1,2-a]pyrazine-6-carboxamide

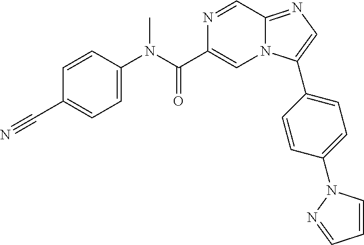 [SMILES: CN(C(=O)c1cn2c(-c3ccc(-n4cccn4)cc3)cnc2cn1)c1ccc(C#N)cc1]

The compound was synthesized using I-59 and an appropriate boronic acid/ester using a procedure similar Suzuki coupling protocol described in 228. 1H NMR (400 MHz, DMSO) δ 8.82 (d, J=1.3 Hz, 1H), 8.74 (d, J=1.3 Hz, 1H), 8.10 (s, 1H), 7.98 (d, J=8.3 Hz, 2H), 7.78-7.67 (m, 5H), 7.40 (dd, J=8.0, 9.9 Hz, 2H), 6.80 (d, J=2.2, 1H), 3.41 (s, 3H). M/Z=420.1 (M+1), r.t=1.86 mins.

### Synthesis of Compound 88

### N-(6-methoxypyridin-3-yl)-N-methyl-3-[4-(trifluoromethyl)phenyl]imidazo[1,2-a]pyrazine-6-carboxamide

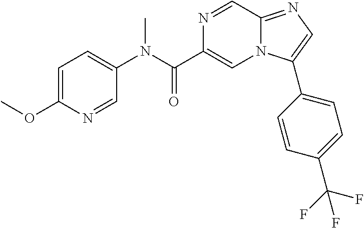 [SMILES: COc1ccc(N(C)C(=O)c2cn3c(-c4ccc(C(F)(F)F)cc4)cnc3cn2)cn1]

The compound was synthesized using a procedure analogous to one use for synthesis of 72 (method II). MS m/z 428.1 (M+H)+; r.t.=2.050.

### Synthesis of Compound 89

### N-(5-cyanopyridin-2-yl)-N-methyl-3-[4-(trifluoromethyl)phenyl]imidazo[1,2-a]pyrazine-6-carboxamide

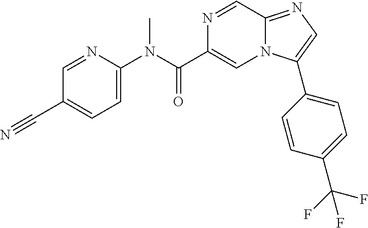 [SMILES: CN(C(=O)c1cn2c(-c3ccc(C(F)(F)F)cc3)cnc2cn1)c1ccc(C#N)cn1]

The compound was synthesized using I-75 and an appropriate boronic acid/ester using a procedure similar Suzuki coupling protocol described in 228. MS m/z 423.1 (M+H)+; r.t.=2.120.

### Synthesis of Compound 90

### N-(4-cyanophenyl)-3-(6-methoxypyridin-3-yl)-N-methylimidazo[1,2-a]pyrazine-6-carboxamide

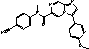 [SMILES: COc1ccc(-c2cnc3cnc(C(=O)N(C)c4ccc(C#N)cc4)cn23)cn1]

The compound was synthesized using I-59 and an appropriate boronic acid/ester using a procedure similar Suzuki coupling protocol described in 228. M/Z=385.2 (M+1), r.t=1.32 mins.

### Synthesis of Compound 91

### -(5-chloropyrazin-2-yl)-N-methyl-3-[4-(trifluoromethyl)phenyl]imidazo[1,2-a]pyrazine-6-carboxamide

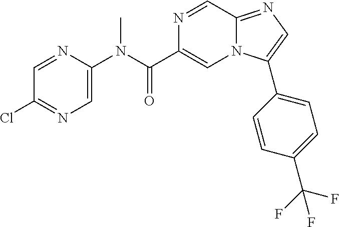 [SMILES: CN(C(=O)c1cn2c(-c3ccc(C(F)(F)F)cc3)cnc2cn1)c1cnc(Cl)cn1]

The compound was synthesized using a procedure analogous to one use for synthesis of 72 (method II). MS m/z 433.1 (M+H)+; r.t.=2.210.

### Synthesis of Compound 92

### N-(4-cyanophenyl)-N-(propan-2-yl)-3-[4-(trifluoromethyl)phenyl]imidazo[1,2-a]pyrazine-6-carboxamide

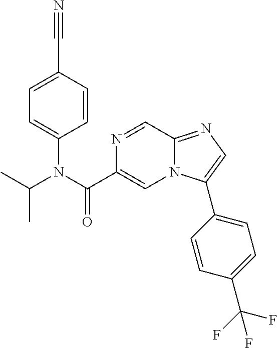 [SMILES: CC(C)N(C(=O)c1cn2c(-c3ccc(C(F)(F)F)cc3)cnc2cn1)c1ccc(C#N)cc1]

The compound was synthesized using a procedure analogous to one use for synthesis of 72 (method II). MS m/z 450.1 (M+H)+; r.t.=2.230.

### Synthesis of Compound 93

### N-(5-chloropyrimidin-2-yl)-N-methyl-3-[4-(trifluoromethyl)phenyl]imidazo[1,2-a]pyrazine-6-carboxamide

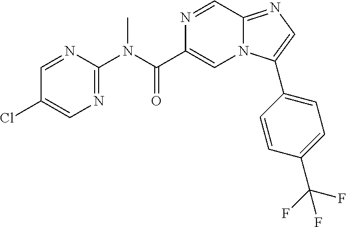 [SMILES: CN(C(=O)c1cn2c(-c3ccc(C(F)(F)F)cc3)cnc2cn1)c1ncc(Cl)cn1]

The compound was synthesized using a procedure analogous to one use for synthesis of 72 (method II). MS m/z 433.1 (M+H)+; r.t.=2.270.

### Synthesis of Compound 94

### N-(6-chloropyridazin-3-yl)-N-methyl-3-[4-(trifluoromethyl)phenyl]imidazo[1,2-a]pyrazine-6-carboxamide

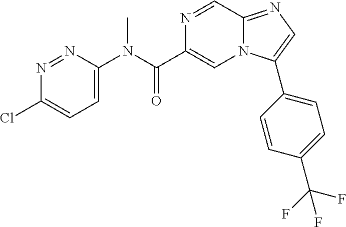 [SMILES: CN(C(=O)c1cn2c(-c3ccc(C(F)(F)F)cc3)cnc2cn1)c1ccc(Cl)nn1]

The compound was synthesized using a procedure analogous to one use for synthesis of 72 (method II). MS m/z 433.1 (M+H)+; r.t.=2.030.

### Synthesis of Compound 95

### N-(5-cyanopyridin-2-yl)-3-[4-(difluoromethyl)phenyl]-N-methylimidazo[1,2-a]pyrazine-6-carboxamide

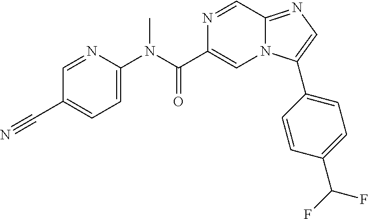 [SMILES: CN(C(=O)c1cn2c(-c3ccc(C(F)F)cc3)cnc2cn1)c1ccc(C#N)cn1]

The compound was synthesized using I-75 and an appropriate boronic acid/ester using a procedure similar Suzuki coupling protocol described in 228. M/Z=405.1 (M+1), r.t=1.54 mins.

### Synthesis of Compound 96

### N-(6-bromopyridazin-3-yl)-N-methyl-3-[4-(trifluoromethyl)phenyl]imidazo[1,2-a]pyrazine-6-carboxamide

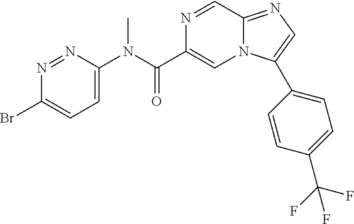 [SMILES: CN(C(=O)c1cn2c(-c3ccc(C(F)(F)F)cc3)cnc2cn1)c1ccc(Br)nn1]

The compound was synthesized using a procedure analogous to one use for synthesis of 72 (method II). MS m/z 477.0 (M+H)+; r.t.=2.090.

### Synthesis of Compound 97

### N-(5-bromopyrazin-2-yl)-N-methyl-3-[4-(trifluoromethyl)phenyl]imidazo[1,2-a]pyrazine-6-carboxamide

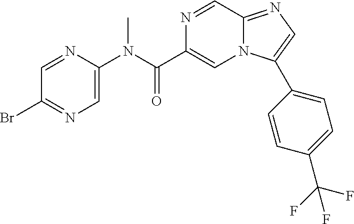 [SMILES: CN(C(=O)c1cn2c(-c3ccc(C(F)(F)F)cc3)cnc2cn1)c1cnc(Br)cn1]

The compound was synthesized using a procedure analogous to one use for synthesis of 72 (method II). MS m/z 477.0 (M+H)+; r.t.=2.270.

### Synthesis of Compound 98

### 3-(4-carbamoylphenyl)-N-(5-cyanopyridin-2-yl)-N-methylimidazo[1,2-a]pyrazine-6-carboxamide

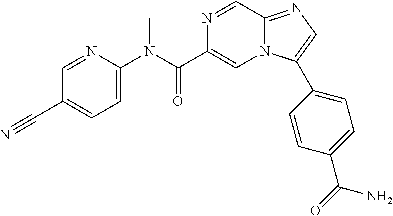 [SMILES: CN(C(=O)c1cn2c(-c3ccc(C(N)=O)cc3)cnc2cn1)c1ccc(C#N)cn1]

The compound was synthesized using I-75 and an appropriate boronic acid/ester using a procedure similar Suzuki coupling protocol described in 228. M/Z=398.1 (M+1), r.t=1.08 mins.

### Synthesis of Compound 99

### N-methyl-N-(pyridin-2-yl)-3-[4-(trifluoromethyl)phenyl]imidazo[1,2-a]pyrazine-6-carboxamide

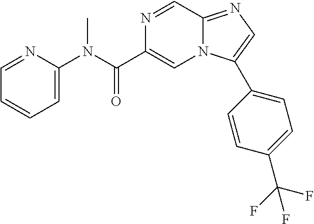 [SMILES: CN(C(=O)c1cn2c(-c3ccc(C(F)(F)F)cc3)cnc2cn1)c1ccccn1]

The compound was synthesized using a procedure analogous to one use for synthesis of 72 (method II). MS m/z 398.1 (M+H)+; r.t.=1.950.

### Synthesis of Compound 100

### N-(6-chloropyridin-3-yl)-3-(4-methoxyphenyl)-N-methylimidazo[1,2-a]pyrazine-6-carboxamide

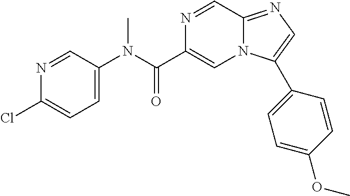 [SMILES: COc1ccc(-c2cnc3cnc(C(=O)N(C)c4ccc(Cl)nc4)cn23)cc1]

The compound was synthesized using I-57 and an appropriate boronic acid/ester using a procedure similar Suzuki coupling protocol described in 228. MS m/z 394.1 (M+H)+; r.t.=1.850.

### Synthesis of Compound 101

### N-(4-fluorophenyl)-N-methyl-3-[4-(trifluoromethyl)phenyl]imidazo[1,2-a]pyrazine-6-carboxamide

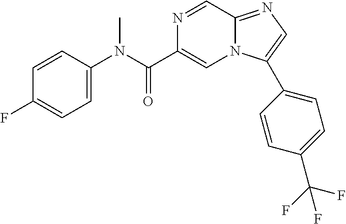 [SMILES: CN(C(=O)c1cn2c(-c3ccc(C(F)(F)F)cc3)cnc2cn1)c1ccc(F)cc1]

The compound was synthesized using a procedure analogous to one use for synthesis of 72 (method II). ¹H NMR (400 MHz, DMSO) δ 8.87 (s, 1H), 8.82 (s, 1H), 8.22 (s, 1H), 7.95 (q, J=8.4, 4H), 7.32 (s, 2H), 7.12 (t, J=8.5, 2H), 3.40 (s, 3H); MS m/z 415.1 (M+H)+; r.t.=2.100.

### Synthesis of Compound 102

### N-(4-chlorophenyl)-3-[4-(difluoromethoxy)phenyl]-N-methylimidazo[1,2-a]pyrazine-6-carboxamide

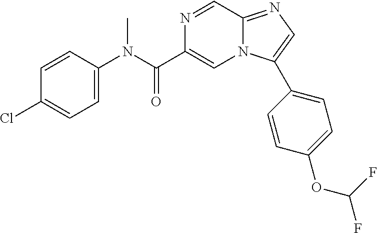 [SMILES: CN(C(=O)c1cn2c(-c3ccc(OC(F)F)cc3)cnc2cn1)c1ccc(Cl)cc1]

The compound was synthesized using I-64 and an appropriate boronic acid/ester using a procedure similar Suzuki coupling protocol described in 228. MS m/z 429.0 (M+H)+; r.t.=1.773.

### Synthesis of Compound 103

### 3-(4-chlorophenyl)-N-(4-fluorophenyl)-N-methylimidazo[1,2-a]pyrazine-6-carboxamide

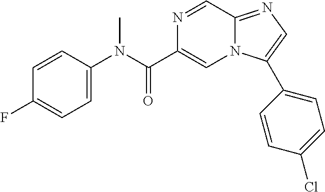 [SMILES: CN(C(=O)c1cn2c(-c3ccc(Cl)cc3)cnc2cn1)c1ccc(F)cc1]

The compound was synthesized using I-64 and an appropriate boronic acid/ester using a procedure similar Suzuki coupling protocol described in 228. ¹H NMR (400 MHz, DMSO) δ 8.83 (s, 1H), 8.73 (s, 1H), 8.12 (s, 1H), 7.76-7.66 (m, 4H), 7.37-7.24 (m, 2H), 7.12 (t, J=8.6, 2H), 3.40 (s, 3H); MS m/z 381.1 (M+H)+; r.t.=2.080.

### Synthesis of Compound 104

### 3-(4-carbamoylphenyl)-N-(4-cyanophenyl)-N-methylimidazo[1,2-a]pyrazine-6-carboxamide

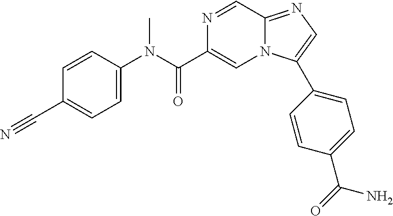 [SMILES: CN(C(=O)c1cn2c(-c3ccc(C(N)=O)cc3)cnc2cn1)c1ccc(C#N)cc1]

The compound was synthesized using I-59 and an appropriate boronic acid/ester using a procedure similar Suzuki coupling protocol described in 228. 1H NMR (400 MHz, DMSO) δ 8.82 (d, J=1.4 Hz, 1H), 8.75 (d, J=1.3, 1H), 8.15 (s, 1H), 8.09 (s, 1H), 8.03 (d, J=8.4 Hz, 2H), 7.77 (d, J=8.4 Hz, 2H), 7.73-7.65 (m, 2H), 7.48 (s, 1H), 7.43-7.36 (m, 2H), 3.39 (s, 3H). M/Z=397.1 (M+1), r.t=1.14 mins.

### Synthesis of Compound 105

### N-(3,4-difluorophenyl)-N-methyl-3-[4-(trifluoromethyl)phenyl]imidazo[1,2-a]pyrazine-6-carboxamide

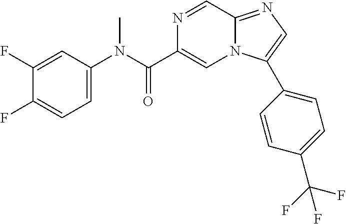 [SMILES: CN(C(=O)c1cn2c(-c3ccc(C(F)(F)F)cc3)cnc2cn1)c1ccc(F)c(F)c1]

The compound was synthesized using I-65 and an appropriate boronic acid/ester using a procedure similar Suzuki coupling protocol described in 228. ¹H NMR (400 MHz, DMSO) δ 8.87 (s, 2H), 8.24 (s, 1H), 8.04-7.90 (m, 4H), 7.54 (ddd, J=2.5, 7.3, 11.7, 1H), 7.33 (dd, J=9.2, 19.3, 1H), 7.13 (s, 1H), 3.41 (s, 3H); MS m/z 433.0 (M+H)+; r.t.=1.837.

### Synthesis of Compound 106

### N-(4-fluorophenyl)-N-methyl-3-(1-methyl-1H-indazol-5-yl)imidazo[1,2-a]pyrazine-6-carboxamide

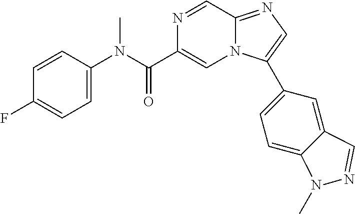 [SMILES: CN(C(=O)c1cn2c(-c3ccc4c(cnn4C)c3)cnc2cn1)c1ccc(F)cc1]

The compound was synthesized using I-66 and an appropriate boronic acid/ester using a procedure similar Suzuki coupling protocol described in 228. M/Z=401.0 (M+1), r.t=1.70 mins.

### Synthesis of Compound 107

### N-(4-fluorophenyl)-N-methyl-3-(1-methyl-1H-indazol-5-yl)imidazo[1,2-a]pyrazine-6-carboxamide

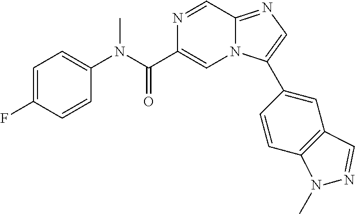 [SMILES: CN(C(=O)c1cn2c(-c3ccc4c(cnn4C)c3)cnc2cn1)c1ccc(F)cc1]

The compound was synthesized using I-64 and an appropriate boronic acid/ester using a procedure similar Suzuki coupling protocol described in 228. MS m/z 411.2 (M+H)+; r.t.=2.230.

### Synthesis of Compound 108

### N-(4-fluorophenyl)-N-methyl-3-(4-methylphenyl)imidazo[1,2-a]pyrazine-6-carboxamide

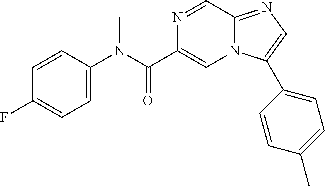 [SMILES: Cc1ccc(-c2cnc3cnc(C(=O)N(C)c4ccc(F)cc4)cn23)cc1]

The compound was synthesized using I-64 and an appropriate boronic acid/ester using a procedure similar Suzuki coupling protocol described in 228. MS m/z 361.2 (M+H)+; r.t.=2.010.

### Synthesis of Compound 109

### N,3-bis(4-chlorophenyl)-N-methylimidazo[1,2-a]pyrazine-6-carboxamide

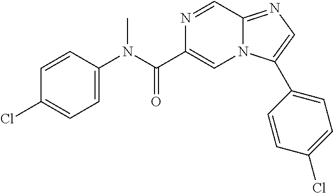 [SMILES: CN(C(=O)c1cn2c(-c3ccc(Cl)cc3)cnc2cn1)c1ccc(Cl)cc1]

The compound was synthesized using I-11 and an appropriate boronic acid/ester using a procedure similar Suzuki coupling protocol described in 228. MS m/z 397.1 (M+H)+; r.t.=2.160.

### Synthesis of Compound 110

### N-(2-methoxyphenyl)-N-methyl-3-[4-(trifluoromethyl)phenyl]imidazo[1,2-a]pyrazine-6-carboxamide

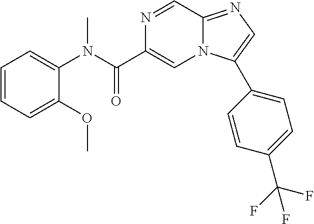 [SMILES: COc1ccccc1N(C)C(=O)c1cn2c(-c3ccc(C(F)(F)F)cc3)cnc2cn1]

The compound was synthesized using a procedure analogous to one use for synthesis of 72 (method II). MS m/z 427.1 (M+H)+; r.t.=2.120.

### Synthesis of Compound 111

### 6-fluoro-1-({3-[4-(trifluoromethyl)phenyl]imidazo[1,2-a]pyrazin-6-yl}carbonyl)-1,2,3,4-tetrahydroquinoline

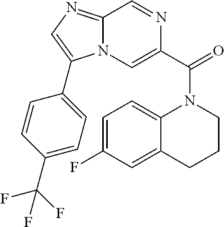 [SMILES: O=C(c1cn2c(-c3ccc(C(F)(F)F)cc3)cnc2cn1)N1CCCc2cc(F)ccc21]

The compound was synthesized using a procedure analogous to one use for synthesis of 72 (method II). MS m/z 441.1 (M+H)+; r.t.=2.300.

### Synthesis of Compound 112

### 3-(4-chlorophenyl)-N-(3,4-difluorophenyl)-N-methylimidazo[1,2-a]pyrazine-6-carboxamide

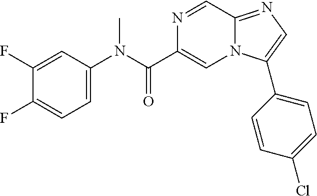 [SMILES: CN(C(=O)c1cn2c(-c3ccc(Cl)cc3)cnc2cn1)c1ccc(F)c(F)c1]

The compound was synthesized using I-65 and an appropriate boronic acid/ester using a procedure similar Suzuki coupling protocol described in 228. MS m/z 399.1 (M+H)+; r.t.=2.140.

### Synthesis of Compound 113

### N-(4-cyanophenyl)-N-methyl-3-(4-methylphenyl)imidazo[1,2-a]pyrazine-6-carboxamide

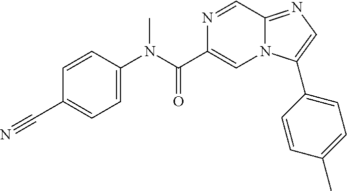 [SMILES: Cc1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. M/Z=368.2 (M+1), r.t=1.94 mins.

### Synthesis of Compound 114

### N-(4-cyanophenyl)-N-ethyl-3-[4-(trifluoromethyl)phenyl]imidazo[1,2-a]pyrazine-6-carboxamide

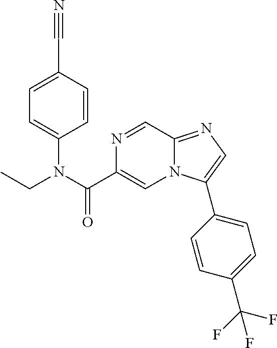 [SMILES: CCN(C(=O)c1cn2c(-c3ccc(C(F)(F)F)cc3)cnc2cn1)c1ccc(C#N)cc1]

The compound was synthesized using a procedure analogous to one use for synthesis of 72 (method II). MS m/z 436.1 (M+H)+; r.t.=2.250.

### Synthesis of Compound 115

### N-(4-cyanophenyl)-N-methyl-3-[4-(propan-2-yl)phenyl]imidazo[1,2-a]pyrazine-6-carboxamide

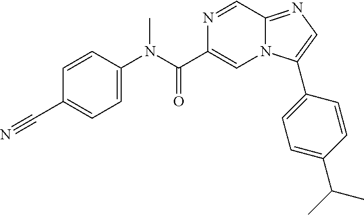 [SMILES: CC(C)c1ccc(-c2cnc3cnc(C(=O)N(C)c4ccc(C#N)cc4)cn23)cc1]

The compound was synthesized using I-57 and an appropriate boronic acid/ester using a procedure similar Suzuki coupling protocol described in 228. MS m/z 396.2 (M+H)+; r.t.=2.300

### Synthesis of Compound 116

### -(4-cyanophenyl)-N-methyl-3-[4-(trifluoromethoxy)phenyl]imidazo[1,2-a]pyrazine-6-carboxamide

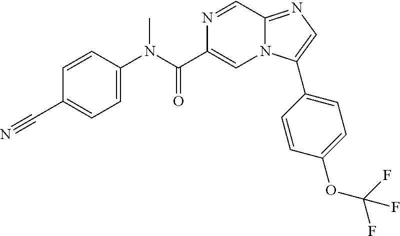 [SMILES: CN(C(=O)c1cn2c(-c3ccc(OC(F)(F)F)cc3)cnc2cn1)c1ccc(C#N)cc1]

The compound was synthesized using I-59 and an appropriate boronic acid/ester using a procedure similar Suzuki coupling protocol described in 228. MS m/z 438.1 (M+H)+; r.t.=2.160.

### Synthesis of Compound 117

### N-(4-cyanophenyl)-N-methyl-3-[4-(methylamino)phenyl]imidazo[1,2-a]pyrazine-6-carboxamide

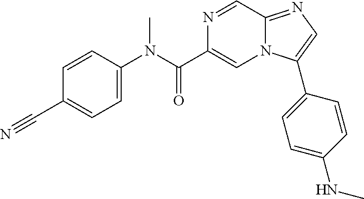 [SMILES: CNc1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. M/Z=383.1 (M+1), r.t=1.24 mins.

### Synthesis of Compound 118

### 3-(4-tert-butylphenyl)-N-(4-cyanophenyl)-N-methylimidazo[1,2-a]pyrazine-6-carboxamide

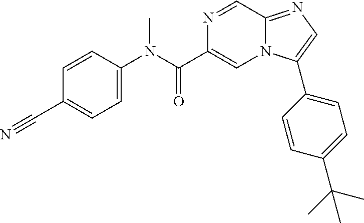 [SMILES: CN(C(=O)c1cn2c(-c3ccc(C(C)(C)C)cc3)cnc2cn1)c1ccc(C#N)cc1]

The compound was synthesized using I-59 and an appropriate boronic acid/ester using a procedure similar Suzuki coupling protocol described in 228. MS m/z 410.2 (M+H)+; r.t.=2.320.

### Synthesis of Compound 119

### N-(2-fluorophenyl)-N-methyl-3-[4-(trifluoromethyl)phenyl]imidazo[1,2-a]pyrazine-6-carboxamide

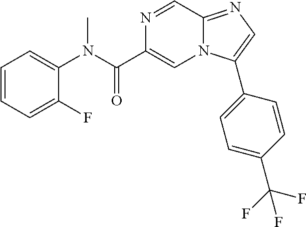 [SMILES: CN(C(=O)c1cn2c(-c3ccc(C(F)(F)F)cc3)cnc2cn1)c1ccccc1F]

The compound was synthesized using a procedure analogous to one use for synthesis of 72 (method II). MS m/z 415.1 (M+H)+; r.t.=2.160.

### Synthesis of Compound 120

### -[4-(1-cyano-1-methylethyl)phenyl]-N-(4-cyanophenyl)-N-methylimidazo[1,2-a]pyrazine-6-carboxamide

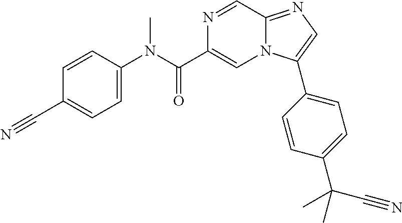 [SMILES: CN(C(=O)c1cn2c(-c3ccc(C(C)(C)C#N)cc3)cnc2cn1)c1ccc(C#N)cc1]

The compound was synthesized using I-59 and an appropriate boronic acid/ester using a procedure similar Suzuki coupling protocol described in 228. MS m/z 421.1 (M+H)+; r.t.=1.769.

### Synthesis of Compound 121

### 3-[4-(dimethylamino)phenyl]-N-(4-fluorophenyl)-N-methylimidazo[1,2-a]pyrazine-6-carboxamide

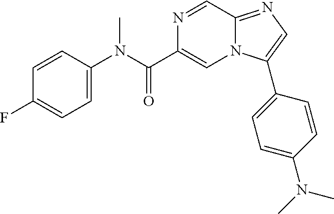 [SMILES: CN(C)c1ccc(-c2cnc3cnc(C(=O)N(C)c4ccc(F)cc4)cn23)cc1]

The compound was synthesized using I-64 and an appropriate boronic acid/ester using a procedure similar Suzuki coupling protocol described in 228. MS m/z 390.1 (M+H)+; r.t.=1.253.

### Synthesis of Compound 122

### N-(4-chlorophenyl)-3-[4-(dimethylamino)phenyl]-N-methylimidazo[1,2-a]pyrazine-6-carboxamide

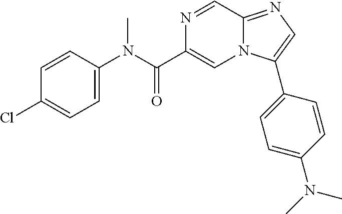 [SMILES: CN(C)c1ccc(-c2cnc3cnc(C(=O)N(C)c4ccc(Cl)cc4)cn23)cc1]

The compound was synthesized using I-11 and an appropriate boronic acid/ester using a procedure similar Suzuki coupling protocol described in 228. MS m/z 406.0 (M+H)+; r.t.=1.383.

### Synthesis of compound 123

### N-(4-cyanophenyl)-N-methyl-3-(1-methyl-1H-indol-5-yl)imidazo[1,2-a]pyrazine-6-carboxamide

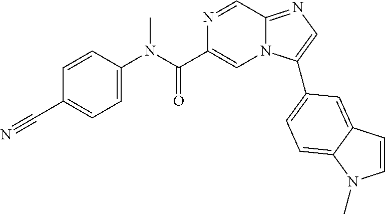 [SMILES: CN(C(=O)c1cn2c(-c3ccc4c(ccn4C)c3)cnc2cn1)c1ccc(C#N)cc1]

The compound was synthesized using I-59 and an appropriate boronic acid/ester using a procedure similar Suzuki coupling protocol described in 228. M/Z=407.2 (M+1), r.t=1.95 mins.

### Synthesis of Compound 124

### 3-(4-aminophenyl)-N-(4-cyanophenyl)-N-methylimidazo[1,2-a]pyrazine-6-carboxamide

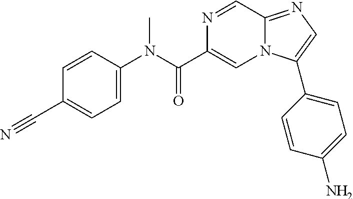 [SMILES: CN(C(=O)c1cn2c(-c3ccc(N)cc3)cnc2cn1)c1ccc(C#N)cc1]

To a solution of 3-bromo-N-(4-cyanophenyl)-N-methylimidazo[1,2-a]pyrazine-6-carboxamide I-59 (1 gm, 2.8 mmol, 1 eq) in THF/water (5:1) 30 mL was added the 4-(4,4,5,5-tetramethyl-1,3,2-dioxaborolan-2-yl)aniline (766 mg, 3.5 mmols, 1.25 eq) followed by DPP-Pd (cat) and K₂HPO₄ (1.21 g, 7 mmol, 2.5 eq). The reaction mixture was heated under microwave condition at 150° C. for 45 mins. The reaction mixture was quenched with water, organics were extracted with ethyl acetate (3×10 mL), followed by drying over sodium sulfate. The crude was then purified using column chromatography to isolate the desired product (0.68 g, 1.84 mmol, 65%). M/Z=369.1 (M+1), r.t=1.00 mins.

### Synthesis of Compound 125

### N-(4-cyanophenyl)-3-[4-(furan-2-yl)phenyl]-N-methylimidazo[1,2-a]pyrazine-6-carboxamide

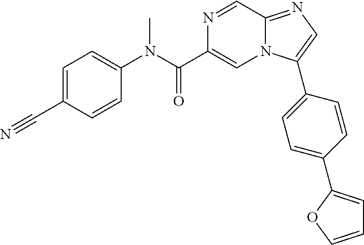 [SMILES: CN(C(=O)c1cn2c(-c3ccc(-c4ccco4)cc3)cnc2cn1)c1ccc(C#N)cc1]

The compound was synthesized using I-59 and an appropriate boronic acid/ester using a procedure similar Suzuki coupling protocol described in 228. M/Z=420.1 (M+1), r.t=1.79 mins.

### Synthesis of Compound 126

### N-(4-cyano-3-fluorophenyl)-N-methyl-3-[4-(trifluoromethyl)phenyl]imidazo[1,2-a]pyrazine-6-carboxamide

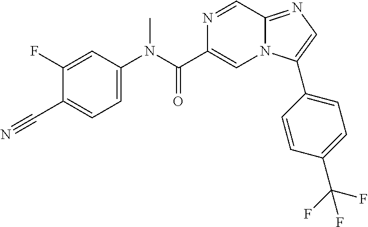 [SMILES: CN(C(=O)c1cn2c(-c3ccc(C(F)(F)F)cc3)cnc2cn1)c1ccc(C#N)c(F)c1]

The compound was synthesized using a procedure analogous to one use for synthesis of 72 (method II). MS m/z 440.1 (M+H)+; r.t.=2.190.

### Synthesis of Compound 127

### N-(3,4-difluorophenyl)-N-methyl-3-(1-methyl-1H-indol-5-yl)imidazo[1,2-a]pyrazine-6-carboxamide

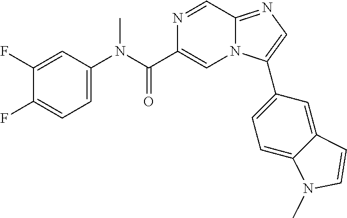 [SMILES: CN(C(=O)c1cn2c(-c3ccc4c(ccn4C)c3)cnc2cn1)c1ccc(F)c(F)c1]

The compound was synthesized using I-65 and an appropriate boronic acid/ester using a procedure similar Suzuki coupling protocol described in 228. MS m/z 418.1 (M+H)+; r.t.=2.100.

### Synthesis of Compound 128

### N-(4-methanesulfonylphenyl)-3-[4-(trifluoromethyl)phenyl]imidazo[1,2-a]pyrazine-6-carboxamide

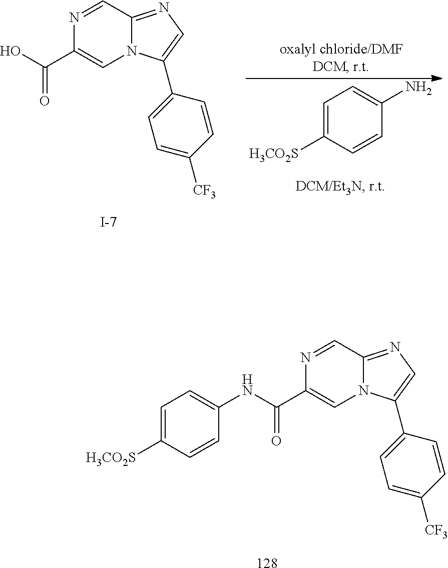

N-(4-(methylsulfonyl)phenyl)-3-(4-(trifluoromethyl)phenyl)imidazo[1,2-a]pyrazine-6-carboxamide was prepared according to the procedure described for the synthesis of N-(4-cyanophenyl)-N-methyl-3-(4-(trifluoromethyl)phenyl)imidazo[1,2-a]pyrazine-6-carboxamide by substituting 4-trifluoromethylaniline for 4-cyano-N-methyl aniline. LC/MS m/z 461.1 (M+H)+; r.t.=2.070.

### Synthesis of Compound 129

### N-(4-cyanophenyl)-3-(4-methanesulfonylphenyl)-N-methylimidazo[1,2-a]pyrazine-6-carboxamide

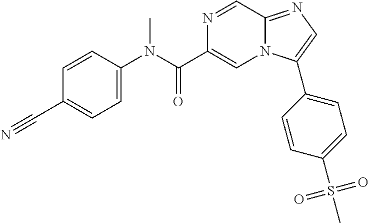 [SMILES: CN(C(=O)c1cn2c(-c3ccc(S(C)(=O)=O)cc3)cnc2cn1)c1ccc(C#N)cc1]

The compound was synthesized using I-59 and an appropriate boronic acid/ester using a procedure similar Suzuki coupling protocol described in 228. M/Z=432.0 (M+1), r.t=1.62 mins.

### Synthesis of Compound 130

### N-(4-cyanophenyl)-3-[4-(1-methoxyethyl)phenyl]-N-methylimidazo[1,2-a]pyrazine-6-carboxamide

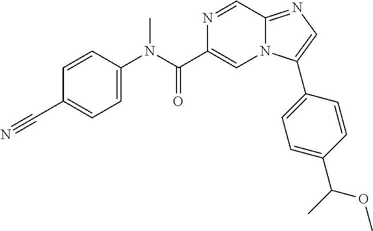 [SMILES: COC(C)c1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. MS m/z 412.1 (M+H)+; r.t.=1.709.

### Synthesis of Compound 131

### N-(4-methanesulfonylphenyl)-N-methyl-3-[4-(1H-pyrazol-5-yl)phenyl]imidazo[1,2-a]pyrazine-6-carboxamide

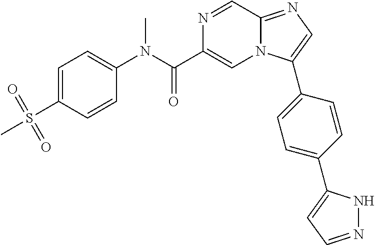 [SMILES: CN(C(=O)c1cn2c(-c3ccc(-c4ccn[nH]4)cc3)cnc2cn1)c1ccc(S(C)(=O)=O)cc1]

The compound was synthesized using I-66 and an appropriate boronic acid/ester using a procedure similar Suzuki coupling protocol described in 228. MS m/z 473.1 (M+H)+; r.t.=1.252.

### Synthesis of Compound 132

### 3-(4-chlorophenyl)-N-(4-cyanophenyl)-N-methylimidazo[1,2-a]pyrazine-6-carboxamide

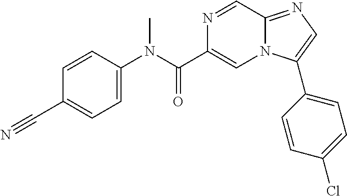 [SMILES: CN(C(=O)c1cn2c(-c3ccc(Cl)cc3)cnc2cn1)c1ccc(C#N)cc1]

The compound was synthesized using I-59 and an appropriate boronic acid/ester using a procedure similar Suzuki coupling protocol described in 228. MS m/z 388.1 (M+H)+; r.t.=2.020.

### Synthesis of Compound 133

### N-(4-cyanophenyl)-N-[2-(oxan-4-yl)ethyl]-3-[4-(trifluoromethyl)phenyl]imidazo[1,2-a]pyrazine-6-carboxamide

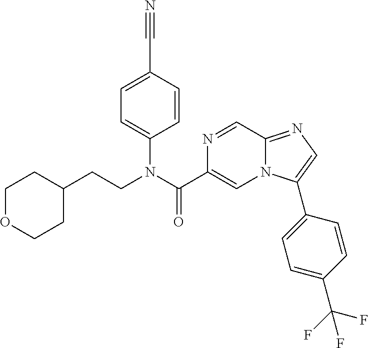 [SMILES: N#Cc1ccc(N(CCC2CCOCC2)C(=O)c2cn3c(-c4ccc(C(F)(F)F)cc4)cnc3cn2)cc1]

The compound was synthesized using a procedure analogous to one use for synthesis of 72 (method II), using intermediate I-31. MS m/z 520.2 (M+H)+; r.t.=2.240.

### Synthesis of Compound 134

### 3-(4-acetylphenyl)-N-(4-cyanophenyl)-N-methylimidazo[1,2-a]pyrazine-6-carboxamide

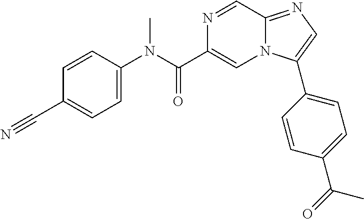 [SMILES: CC(=O)c1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. M/Z=396.1 (M+1), r.t=1.41 mins.

### Synthesis of Compound 135

### N-(3,4-difluorophenyl)-N-methyl-3-(4-methylphenyl)imidazo[1,2-a]pyrazine-6-carboxamide

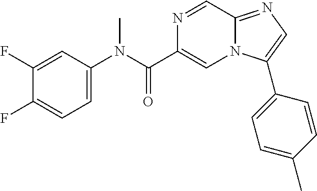 [SMILES: Cc1ccc(-c2cnc3cnc(C(=O)N(C)c4ccc(F)c(F)c4)cn23)cc1]

The compound was synthesized using I-65 and an appropriate boronic acid/ester using a procedure similar Suzuki coupling protocol described in 228. MS m/z 379.2 (M+H)+; r.t.=2.050.

### Synthesis of Compound 136

### N-(4-fluorophenyl)-N-methyl-3-(1-methyl-1H-indol-5-yl)imidazo[1,2-a]pyrazine-6-carboxamide

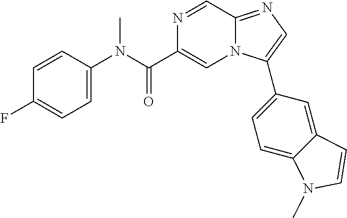 [SMILES: CN(C(=O)c1cn2c(-c3ccc4c(ccn4C)c3)cnc2cn1)c1ccc(F)cc1]

The compound was synthesized using I-64 and an appropriate boronic acid/ester using a procedure similar Suzuki coupling protocol described in 228. MS m/z 400.2 (M+H)+; r.t.=2.020.

### Synthesis of Compound 137

### N-(3-fluorophenyl)-N-methyl-3-[4-(trifluoromethyl)phenyl]imidazo[1,2-a]pyrazine-6-carboxamide

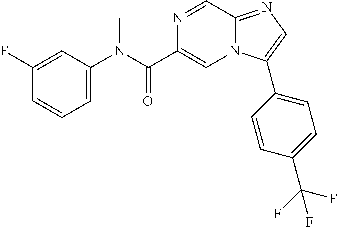 [SMILES: CN(C(=O)c1cn2c(-c3ccc(C(F)(F)F)cc3)cnc2cn1)c1cccc(F)c1]

The compound was synthesized using a procedure analogous to one use for synthesis of 72 (method II). MS m/z 415.1 (M+H)+; r.t.=2.110.

### Synthesis of Compound 138

### N-(4-cyanophenyl)-N-methyl-3-[4-(2,2,2-trifluoroethoxy)phenyl]imidazo[1,2-a]pyrazine-6-carboxamide

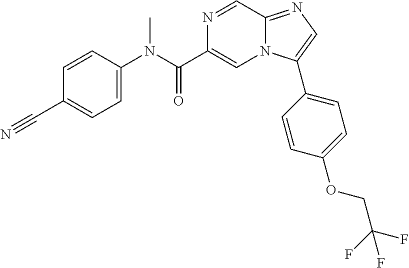 [SMILES: CN(C(=O)c1cn2c(-c3ccc(OCC(F)(F)F)cc3)cnc2cn1)c1ccc(C#N)cc1]

The compound was synthesized using I-59 and an appropriate boronic acid/ester using a procedure similar Suzuki coupling protocol described in 228. M/Z=452.0 (M+1), r.t=1.67 mins.

### Synthesis of Compound 139

### N-(4-methoxyphenyl)-N-methyl-3-[4-(trifluoromethyl)phenyl]imidazo[1,2-a]pyrazine-6-carboxamide

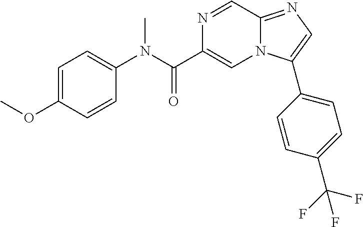 [SMILES: COc1ccc(N(C)C(=O)c2cn3c(-c4ccc(C(F)(F)F)cc4)cnc3cn2)cc1]

The compound was synthesized using a procedure analogous to one use for synthesis of 72 (method II). MS m/z 427.1 (M+H)+; r.t.=2.110.

### Synthesis of Compound 140

### N-(4-cyanophenyl)-3-[2-fluoro-4-(trifluoromethyl)phenyl]-N-methylimidazo[1,2-a]pyrazine-6-carboxamide

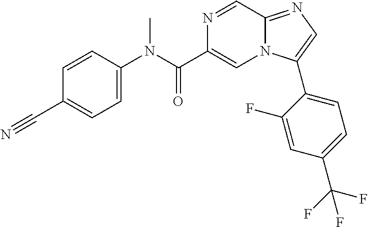 [SMILES: CN(C(=O)c1cn2c(-c3ccc(C(F)(F)F)cc3F)cnc2cn1)c1ccc(C#N)cc1]

The compound was synthesized using I-59 and an appropriate boronic acid/ester using a procedure similar Suzuki coupling protocol described in 228. M/Z=440.1 (M+1), r.t=2.13 mins.

### Synthesis of Compound 141

### N-(4-chlorophenyl)-N-methyl-3-(1-methyl-1H-indol-5-yl)imidazo[1,2-a]pyrazine-6-carboxamide

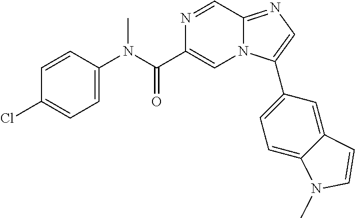 [SMILES: CN(C(=O)c1cn2c(-c3ccc4c(ccn4C)c3)cnc2cn1)c1ccc(Cl)cc1]

The compound was synthesized using I-11 and an appropriate boronic acid/ester using a procedure similar Suzuki coupling protocol described in 228. MS m/z 416.1 (M+H)+; r.t.=2.130.

### Synthesis of Compound 142

### 3-[4-(difluoromethoxy)phenyl]-N-(3,4-difluorophenyl)-N-methylimidazo[1,2-a]pyrazine-6-carboxamide

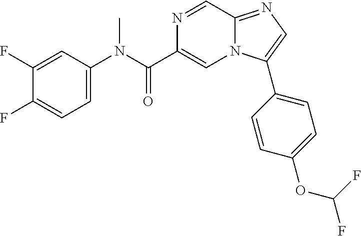 [SMILES: CN(C(=O)c1cn2c(-c3ccc(OC(F)F)cc3)cnc2cn1)c1ccc(F)c(F)c1]

The compound was synthesized using I-65 and an appropriate boronic acid/ester using a procedure similar Suzuki coupling protocol described in 228. MS m/z 431.1 (M+H)+; r.t.=1.695.

### Synthesis of Compound 143

### 3-(1H-1,3-benzodiazol-5-yl)-N-(4-cyanophenyl)-N-methylimidazo[1,2-a]pyrazine-6-carboxamide

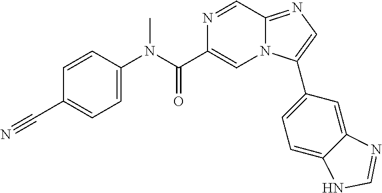 [SMILES: CN(C(=O)c1cn2c(-c3ccc4[nH]cnc4c3)cnc2cn1)c1ccc(C#N)cc1]

The compound was synthesized using I-59 and an appropriate boronic acid/ester using a procedure similar Suzuki coupling protocol described in 228. M/Z=394.2 (M+1), r.t=1.20 mins.

### Synthesis of Compound 144

### N-(3,4-difluorophenyl)-N-methyl-3-[4-(1H-pyrazol-1-yl)phenyl]imidazo[1,2-a]pyrazine-6-carboxamide

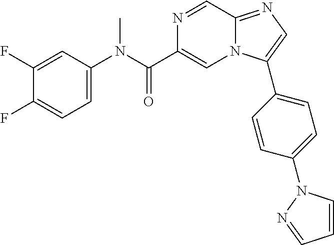 [SMILES: CN(C(=O)c1cn2c(-c3ccc(-n4cccn4)cc3)cnc2cn1)c1ccc(F)c(F)c1]

The compound was synthesized using I-65 and an appropriate boronic acid/ester using a procedure similar Suzuki coupling protocol described in 228. MS m/z 431.1 (M+H)+; r.t.=1.815.

### Synthesis of Compound 145

### N-(4-cyanophenyl)-3-(1H-indazol-5-yl)-N-methylimidazo[1,2-a]pyrazine-6-carboxamide

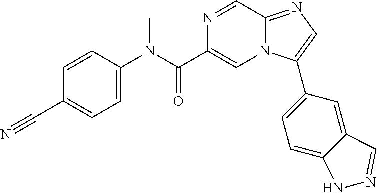 [SMILES: CN(C(=O)c1cn2c(-c3ccc4[nH]ncc4c3)cnc2cn1)c1ccc(C#N)cc1]

The compound was synthesized using I-59 and an appropriate boronic acid/ester using a procedure similar Suzuki coupling protocol described in 228. M/Z=394.1 (M+1), r.t=1.54 mins.

### Synthesis of Compound 146

### N-(6-cyanopyridin-3-yl)-N-methyl-3-[4-(trifluoromethyl)phenyl]imidazo[1,2-a]pyrazine-6-carboxamide

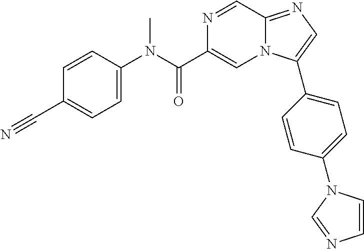 [SMILES: CN(C(=O)c1cn2c(-c3ccc(-n4ccnc4)cc3)cnc2cn1)c1ccc(C#N)cc1]

The compound was synthesized using I-59 and an appropriate boronic acid/ester using a procedure similar Suzuki coupling protocol described in 228. M/Z=420.1 (M+1), r.t=1.12 mins.

### Synthesis of Compound 147

### N-(6-cyanopyridin-3-yl)-N-methyl-3-[4-(trifluoromethyl)phenyl]imidazo[1,2-a]pyrazine-6-carboxamide

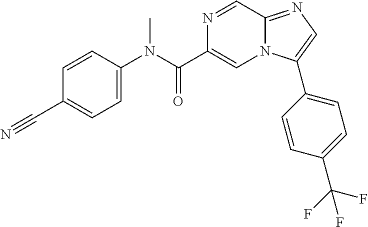 [SMILES: CN(C(=O)c1cn2c(-c3ccc(C(F)(F)F)cc3)cnc2cn1)c1ccc(C#N)cc1]

The compound was synthesized using a procedure analogous to one use for synthesis of 72 (method II), using intermediate I-37. MS m/z 423.1 (M+H)+; r.t.=2.050.

### Synthesis of Compound 148

### N-(4-cyanophenyl)-N-methyl-3-(2-methyl-2H-indazol-5-yl)imidazo[1,2-a]pyrazine-6-carboxamide

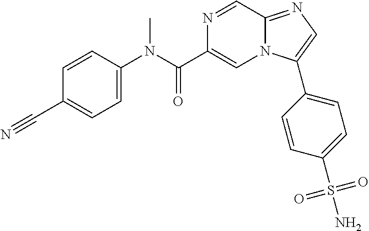 [SMILES: CN(C(=O)c1cn2c(-c3ccc(S(N)(=O)=O)cc3)cnc2cn1)c1ccc(C#N)cc1]

The compound was synthesized using I-59 and an appropriate boronic acid/ester using a procedure similar Suzuki coupling protocol described in 228. M/Z=433.0 (M+1), r.t=1.21 mins.

### Synthesis of Compound 149

### N-(4-cyanophenyl)-N-methyl-3-(2-methyl-2H-indazol-5-yl)imidazo[1,2-a]pyrazine-6-carboxamide

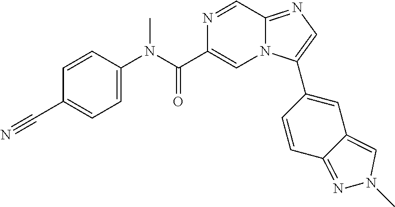 [SMILES: CN(C(=O)c1cn2c(-c3ccc4nn(C)cc4c3)cnc2cn1)c1ccc(C#N)cc1]

The compound was synthesized using I-59 and an appropriate boronic acid/ester using a procedure similar Suzuki coupling protocol described in 228. M/Z=408.1 (M+1), r.t=1.36 mins.

### Synthesis of Compound 150

### N-(4-cyanophenyl)-N-methyl-3-[2-methyl-4-(trifluoromethyl)phenyl]imidazo[1,2-a]pyrazine-6-carboxamide

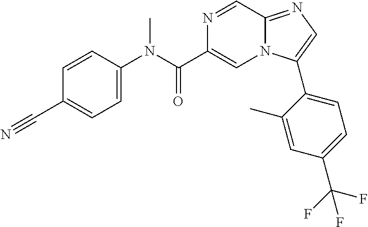 [SMILES: Cc1cc(C(F)(F)F)ccc1-c1cnc2cnc(C(=O)N(C)c3ccc(C#N)cc3)cn12]

The compound was synthesized using I-59 and an appropriate boronic acid/ester using a procedure similar Suzuki coupling protocol described in 228. M/Z=436.1 (M+1), r.t=2.10 mins.

### Synthesis of Compound 151

### N-(4-fluorophenyl)-N-methyl-3-[4-(1H-pyrazol-1-yl)phenyl]imidazo[1,2-a]pyrazine-6-carboxamide

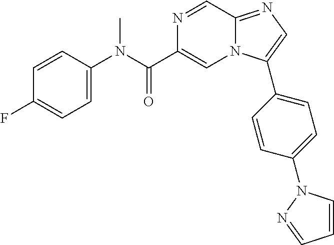 [SMILES: CN(C(=O)c1cn2c(-c3ccc(-n4cccn4)cc3)cnc2cn1)c1ccc(F)cc1]

The compound was synthesized using I-64 and an appropriate boronic acid/ester using a procedure similar Suzuki coupling protocol described in 228. MS m/z 413.1 (M+H)+; r.t.=1.725.

### Synthesis of Compound 152

### N-(4-cyanophenyl)-N-methyl-3-[4-(propane-2-sulfonyl)phenyl]imidazo[1,2-a]pyrazine-6-carboxamide

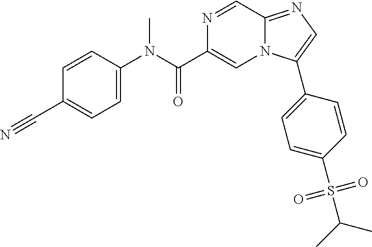 [SMILES: CC(C)S(=O)(=O)c1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. M/Z=460.1 (M+1), r.t=1.83 mins.

### Synthesis of Compound 153

### N-(4-cyanophenyl)-3-(4-fluorophenyl)-N-methylimidazo[1,2-a]pyrazine-6-carboxamide

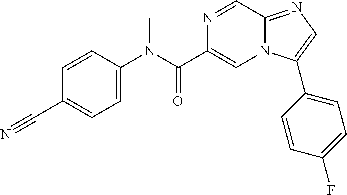 [SMILES: CN(C(=O)c1cn2c(-c3ccc(F)cc3)cnc2cn1)c1ccc(C#N)cc1]

The compound was synthesized using I-59 and an appropriate boronic acid/ester using a procedure similar Suzuki coupling protocol described in 228. M/Z=372.1 (M+1), r.t=1.89 mins.

### Synthesis of Compound 154

### N-(4-cyanophenyl)-3-(1H-indazol-6-yl)-N-methylimidazo[1,2-a]pyrazine-6-carboxamide

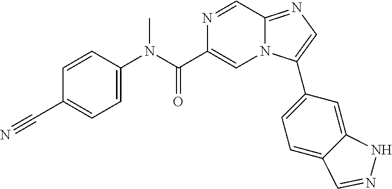 [SMILES: CN(C(=O)c1cn2c(-c3ccc4cn[nH]c4c3)cnc2cn1)c1ccc(C#N)cc1]

The compound was synthesized using I-59 and an appropriate boronic acid/ester using a procedure similar Suzuki coupling protocol described in 228. M/Z=394.1 (M+1), r.t=1.62 mins.

### Synthesis of Compound 155

### 3-(4-chloro-3-cyanophenyl)-N-(4-cyanophenyl)-N-methylimidazo[1,2-a]pyrazine-6-carboxamide

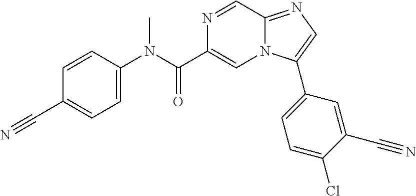 [SMILES: CN(C(=O)c1cn2c(-c3ccc(Cl)c(C#N)c3)cnc2cn1)c1ccc(C#N)cc1]

The compound was synthesized using I-59 and an appropriate boronic acid/ester using a procedure similar Suzuki coupling protocol described in 228. M/Z=413.1 (M+1), r.t=1.93 mins.

### Synthesis of Compound 156

### N-(4-cyanophenyl)-N-methyl-3-[2-(propan-2-yl)-1,3-thiazol-4-yl]imidazo[1,2-a]pyrazine-6-carboxamide

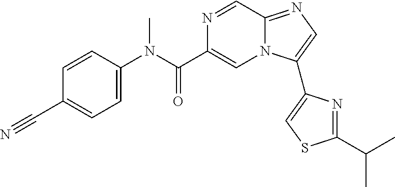 [SMILES: CC(C)c1nc(-c2cnc3cnc(C(=O)N(C)c4ccc(C#N)cc4)cn23)cs1]

The compound was synthesized using I-59 and an appropriate boronic acid/ester using a procedure similar Suzuki coupling protocol described in 228. M/Z=403.1 (M+1), r.t=1.94 mins.

### Synthesis of Compound 157

### N-(4-cyanophenyl)-N-methyl-3-(quinolin-6-yl)imidazo[1,2-a]pyrazine-6-carboxamide

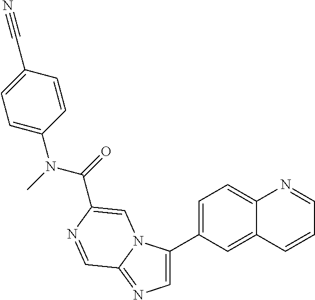 [SMILES: CN(C(=O)c1cn2c(-c3ccc4ncccc4c3)cnc2cn1)c1ccc(C#N)cc1]

The compound was synthesized using I-59 and an appropriate boronic acid/ester using a procedure similar Suzuki coupling protocol described in 228. M/Z=405.1 (M+1), r.t=1.14 mins.

### Synthesis of Compound 158

### N-(4-cyanophenyl)-3-[4-(cyclopropylcarbamoyl)phenyl]-N-methylimidazo[1,2-a]pyrazine-6-carboxamide

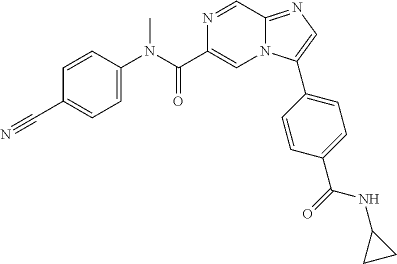 [SMILES: CN(C(=O)c1cn2c(-c3ccc(C(=O)NC4CC4)cc3)cnc2cn1)c1ccc(C#N)cc1]

The compound was synthesized using I-59 and an appropriate boronic acid/ester using a procedure similar Suzuki coupling protocol described in 228. M/Z=437.1 (M+1), r.t=1.29 mins.

### Synthesis of Compound 159

### N-(3,4-difluorophenyl)-3-[4-(dimethylamino)phenyl]-N-methylimidazo[1,2-a]pyrazine-6-carboxamide

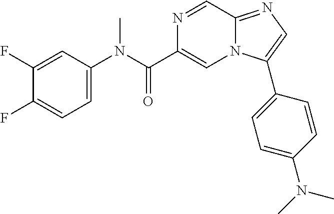 [SMILES: CN(C)c1ccc(-c2cnc3cnc(C(=O)N(C)c4ccc(F)c(F)c4)cn23)cc1]

The compound was synthesized using I-65 and an appropriate boronic acid/ester using a procedure similar Suzuki coupling protocol described in 228. MS m/z 408.1 (M+H)+; r.t.=1.357.

### Synthesis of Compound 160

### N-(4-cyanophenyl)-3-[4-(3,5-dimethyl-1H-pyrazol-1-yl)phenyl]-N-methylimidazo[1,2-a]pyrazine-6-carboxamide

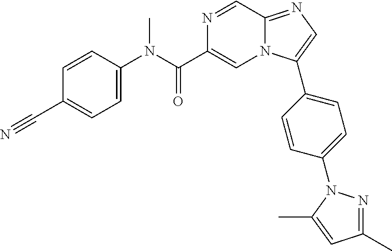 [SMILES: Cc1cc(C)n(-c2ccc(-c3cnc4cnc(C(=O)N(C)c5ccc(C#N)cc5)cn34)cc2)n1]

The compound was synthesized using I-59 and an appropriate boronic acid/ester using a procedure similar Suzuki coupling protocol described in 228. M/Z=448.1 (M+1), r.t=1.65 mins.

### Synthesis of Compound 161

### 3-(4-carbamoylphenyl)-N-(6-chloropyridin-3-yl)-N-methylimidazo[1,2-a]pyrazine-6-carboxamide

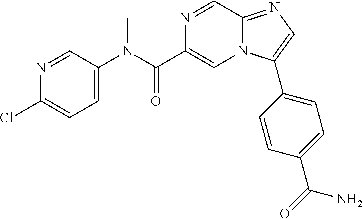 [SMILES: CN(C(=O)c1cn2c(-c3ccc(C(N)=O)cc3)cnc2cn1)c1ccc(Cl)nc1]

The compound was synthesized using I-57 and an appropriate boronic acid/ester using a procedure similar Suzuki coupling protocol described in 228. MS m/z 407.1 (M+H)+; r.t.=1.096.

### Synthesis of Compound 162

### N-(4-cyanophenyl)-N-methyl-3-[4-(propan-2-yloxy)phenyl]imidazo[1,2-a]pyrazine-6-carboxamide

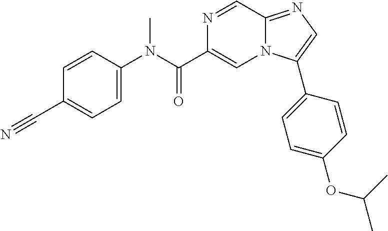 [SMILES: CC(C)Oc1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. M/Z=412.2 (M+1), r.t=2.11 mins.

### Synthesis of Compound 163

### N-(4-cyanophenyl)-N-methyl-3-[4-(5-methyl-1,3,4-oxadiazol-2-yl)phenyl]imidazo[1,2-a]pyrazine-6-carboxamide

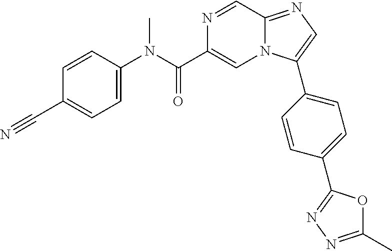 [SMILES: Cc1nnc(-c2ccc(-c3cnc4cnc(C(=O)N(C)c5ccc(C#N)cc5)cn34)cc2)o1]

The compound was synthesized using I-59 and an appropriate boronic acid/ester using a procedure similar Suzuki coupling protocol described in 228. M/Z=436.1 (M+1), r.t=1.42 mins.

### Synthesis of Compound 164

### 3-[4-(dimethylamino)phenyl]-N-(4-methanesulfonylphenyl)-N-methylimidazo[1,2-a]pyrazine-6-carboxamide

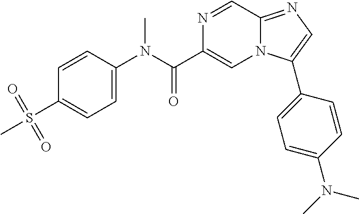

The compound was synthesized using I-66 and an appropriate boronic acid/ester using a procedure similar Suzuki coupling protocol described in 228. MS m/z 450.1 (M+H)+; r.t.=1.103.

### Synthesis of Compound 165

### N-(4-cyanophenyl)-3-[4-(2,5-dioxoimidazolidin-4-yl)phenyl]-N-methylimidazo[1,2-a]pyrazine-6-carboxamide

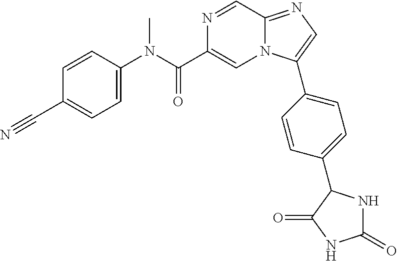 [SMILES: CN(C(=O)c1cn2c(-c3ccc(C4NC(=O)NC4=O)cc3)cnc2cn1)c1ccc(C#N)cc1]

The compound was synthesized using I-59 and an appropriate boronic acid/ester using a procedure similar Suzuki coupling protocol described in 228. M/Z=452.1 (M+1), r.t=1.21 mins.

### Synthesis of Compound 166

### methyl 4-{6-[(4-cyanophenyl)(methyl)carbamoyl]imidazo[1,2-a]pyrazin-3-yl}benzoate

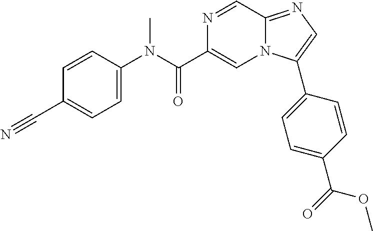 [SMILES: COC(=O)c1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. M/Z=412.1 (M+1), r.t=1.67 mins.

### Synthesis of Compound 167

### N-(4-cyanophenyl)-3-(isoquinolin-6-yl)-N-methylimidazo[1,2-a]pyrazine-6-carboxamide

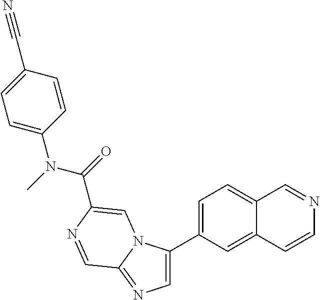 [SMILES: CN(C(=O)c1cn2c(-c3ccc4cnccc4c3)cnc2cn1)c1ccc(C#N)cc1]

The compound was synthesized using I-59 and an appropriate boronic acid/ester using a procedure similar Suzuki coupling protocol described in 228. M/Z=405.1 (M+1), r.t=1.13 mins.

### Synthesis of Compound 168

### N-(4-cyanophenyl)-N-methyl-3-(1-oxo-1,2-dihydroisoquinolin-6-yl)imidazo[1,2-a]pyrazine-6-carboxamide

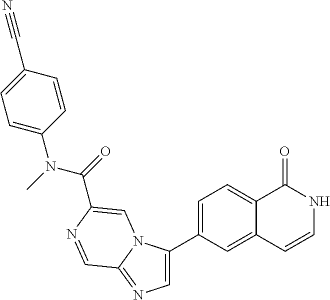 [SMILES: CN(C(=O)c1cn2c(-c3ccc4c(=O)[nH]ccc4c3)cnc2cn1)c1ccc(C#N)cc1]

The compound was synthesized using I-59 and I-26 using a procedure similar Suzuki coupling protocol described in 228. M/Z=421.1 (M+1), r.t=1.17 mins.

### Synthesis of Compound 169

### N-(4-cyanophenyl)-3-[3-fluoro-4-(trifluoromethyl)phenyl]-N-methylimidazo[1,2-a]pyrazine-6-carbox

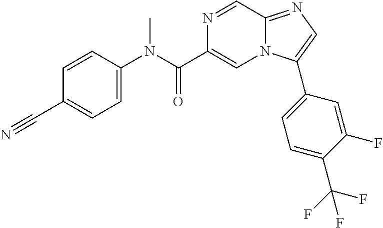 [SMILES: CN(C(=O)c1cn2c(-c3ccc(C(F)(F)F)c(F)c3)cnc2cn1)c1ccc(C#N)cc1]

The compound was synthesized using I-59 and an appropriate boronic acid/ester using a procedure similar Suzuki coupling protocol described in 228 . . . M/Z=413.2 (M+1), r.t=2.03 mins.

### Synthesis of Compound 170

### N-(4-cyanophenyl)-N-methyl-3-(6-propoxypyridin-3-yl)imidazo[1,2-a]pyrazine-6-carboxamide

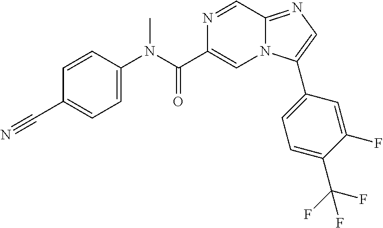 [SMILES: CN(C(=O)c1cn2c(-c3ccc(C(F)(F)F)c(F)c3)cnc2cn1)c1ccc(C#N)cc1]

The compound was synthesized using I-59 and an appropriate boronic acid/ester using a procedure similar Suzuki coupling protocol described in 228. M/Z=413.2 (M+1), r.t=2.03 mins.

### Synthesis of Compound 171

### N-methyl-N-[2-(trifluoromethyl)phenyl]-3-[4-(trifluoromethyl)phenyl]imidazo[1,2-a]pyrazine-6-carboxamide

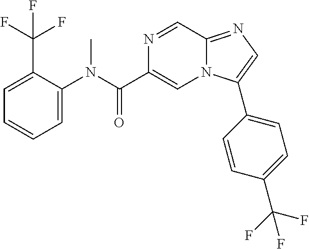 [SMILES: CN(C(=O)c1cn2c(-c3ccc(C(F)(F)F)cc3)cnc2cn1)c1ccccc1C(F)(F)F]

The compound was synthesized using a procedure analogous to one use for synthesis of 72 (method II). MS m/z 465.1 (M+H)+; r.t.=2.390.

### Synthesis of Compound 172

### 3-(4-carbamoylphenyl)-N-(4-methanesulfonylphenyl)-N-methylimidazo[1,2-a]pyrazine-6-carboxamide

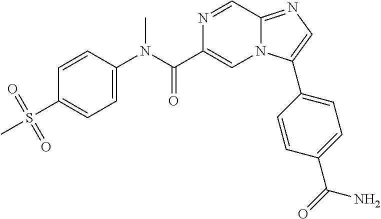 [SMILES: CN(C(=O)c1cn2c(-c3ccc(C(N)=O)cc3)cnc2cn1)c1ccc(S(C)(=O)=O)cc1]

The compound was synthesized using I-66 and an appropriate boronic acid/ester using a procedure similar Suzuki coupling protocol described in 228. MS m/z 450.0 (M+H)+; r.t.=1.030.

### Synthesis of Compound 173

### N-methyl-N-[4-(trifluoromethoxy)phenyl]-3-[4-(trifluoromethyl)phenyl]imidazo[1,2-a]pyrazine-6-carboxamide

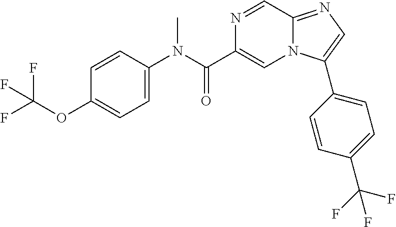 [SMILES: CN(C(=O)c1cn2c(-c3ccc(C(F)(F)F)cc3)cnc2cn1)c1ccc(OC(F)(F)F)cc1]

The compound was synthesized using a procedure analogous to one use for synthesis of 72 (method II). MS m/z 481.1 (M+H)+; r.t.=2.420.

### Synthesis of Compound 174

### N-methyl-N-(1-methyl-1H-imidazol-2-yl)-3-[4-(trifluoromethyl)phenyl]imidazo[1,2-a]pyrazine-6-carboxamide

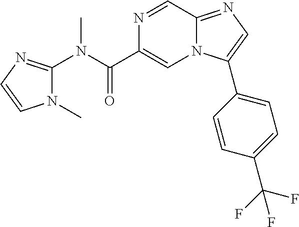 [SMILES: CN(C(=O)c1cn2c(-c3ccc(C(F)(F)F)cc3)cnc2cn1)c1nccn1C]

The compound was synthesized using a procedure analogous to one use for synthesis of 72 (method II). MS m/z 401.2 (M+H)+; r.t.=1.390.

### Synthesis of Compound 175

### N-(4-cyanophenyl)-N-methyl-3-[3-(trifluoromethyl)phenyl]imidazo[1,2-a]pyrazine-6-carboxamide

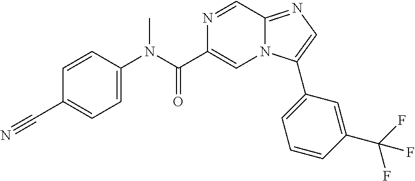 [SMILES: CN(C(=O)c1cn2c(-c3cccc(C(F)(F)F)c3)cnc2cn1)c1ccc(C#N)cc1]

The compound was synthesized using I-59 and an appropriate boronic acid/ester using a procedure similar Suzuki coupling protocol described in 228. M/Z=422.1 (M+1), r.t=1.76 mins.

### Synthesis of Compound 176

### 5-fluoro-1-({3-[4-(trifluoromethyl)phenyl]imidazo[1,2-a]pyrazin-6-yl}carbonyl)-2,3-dihydro-1H-indole

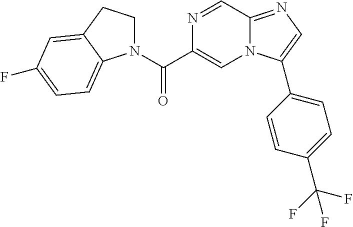 [SMILES: O=C(c1cn2c(-c3ccc(C(F)(F)F)cc3)cnc2cn1)N1CCc2cc(F)ccc21]

The compound was synthesized using a procedure analogous to one use for synthesis of 72 (method II). MS m/z 427.1 (M+H)+; r.t.=2.430.

### Synthesis of Compound 177

### N-(4-cyanophenyl)-N-methyl-3-(4-phenoxyphenyl)imidazo[1,2-a]pyrazine-6-carboxamide

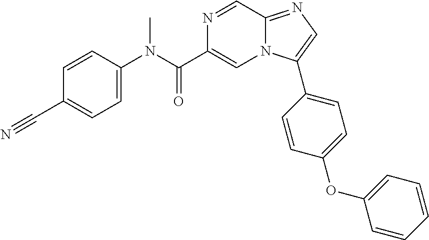 [SMILES: CN(C(=O)c1cn2c(-c3ccc(Oc4ccccc4)cc3)cnc2cn1)c1ccc(C#N)cc1]

The compound was synthesized using I-59 and an appropriate boronic acid/ester using a procedure similar Suzuki coupling protocol described in 228. M/Z=446.1 (M+1), r.t=2.24 mins.

### Synthesis of Compound 178

### 3-(1-benzyl-1H-pyrazol-4-yl)-N-(4-cyanophenyl)-N-methylimidazo[1,2-a]pyrazine-6-carboxamide

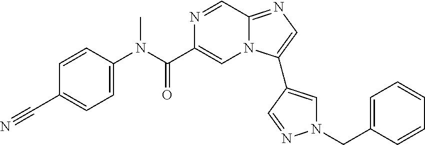 [SMILES: CN(C(=O)c1cn2c(-c3cnn(Cc4ccccc4)c3)cnc2cn1)c1ccc(C#N)cc1]

The compound was synthesized using I-59 and an appropriate boronic acid/ester using a procedure similar Suzuki coupling protocol described in 228. M/Z 434.1 (M+1), r.t=1.59 mins.

### Synthesis of Compound 179

### 3-[4-chloro-2-(trifluoromethyl)phenyl]-N-(4-cyanophenyl)-N-methylimidazo[1,2-a]pyrazine-6-carboxamide

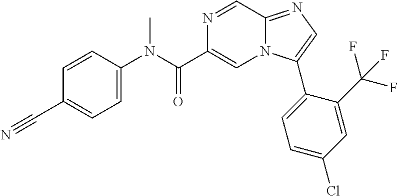 [SMILES: CN(C(=O)c1cn2c(-c3ccc(Cl)cc3C(F)(F)F)cnc2cn1)c1ccc(C#N)cc1]

The compound was synthesized using I-59 and an appropriate boronic acid/ester using a procedure similar Suzuki coupling protocol described in 228. M/Z=456.1.1 (M+1), r.t=2.21 mins.

### Synthesis of Compound 180

### N-(4-cyanophenyl)-N-methyl-3-[4-(1H-1,2,3,4-tetrazol-5-yl)phenyl]imidazo[1,2-a]pyrazine-6-carboxamide

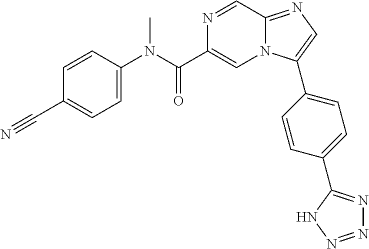 [SMILES: CN(C(=O)c1cn2c(-c3ccc(-c4nnn[nH]4)cc3)cnc2cn1)c1ccc(C#N)cc1]

The compound was synthesized using I-59 and an appropriate boronic acid/ester using a procedure similar Suzuki coupling protocol described in 228. M/Z=422.1 (M+1), r.t=1.35 mins.

### Synthesis of Compound 181

### N-(4-cyanophenyl)-N-methyl-3-[4-(pyrrolidin-1-yl)phenyl]imidazo[1,2-a]pyrazine-6-carboxamide

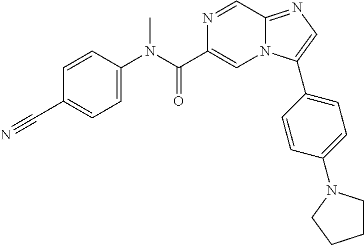 [SMILES: CN(C(=O)c1cn2c(-c3ccc(N4CCCC4)cc3)cnc2cn1)c1ccc(C#N)cc1]

The compound was synthesized using I-59 and an appropriate boronic acid/ester using a procedure similar Suzuki coupling protocol described in 228. M/Z=423.1 (M+1), r.t=1.79 mins.

### Synthesis of Compound 182

### N-(5-cyanopyridin-2-yl)-N-methyl-3-[4-(1H-pyrazol-5-yl)phenyl]imidazo[1,2-a]pyrazine-6-carboxamide

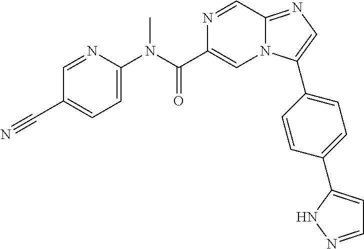 [SMILES: CN(C(=O)c1cn2c(-c3ccc(-c4ccn[nH]4)cc3)cnc2cn1)c1ccc(C#N)cn1]

The compound was synthesized using I-75 and an appropriate boronic acid/ester using a procedure similar Suzuki coupling protocol described in 228. M/Z=421.1 (M+1), r.t=1.49 mins.

### Synthesis of Compound 183

### N-(3-bromophenyl)-N-methyl-3-[4-(trifluoromethyl)phenyl]imidazo[1,2-a]pyrazine-6-carboxamide

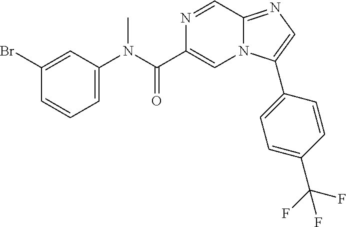 [SMILES: CN(C(=O)c1cn2c(-c3ccc(C(F)(F)F)cc3)cnc2cn1)c1cccc(Br)c1]

The compound was synthesized using a procedure analogous to one use for synthesis of 72 (method II). MS m/z 475.0 (M+H)+; r.t.=1.814.

### Synthesis of Compound 184

### N-(4-cyanophenyl)-N-methyl-3-(5-sulfamoylpyridin-3-yl)imidazo[1,2-a]pyrazine-6-carboxamide

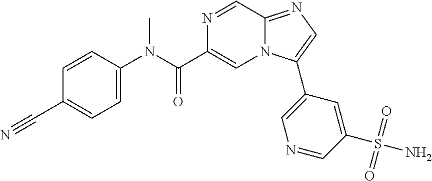 [SMILES: CN(C(=O)c1cn2c(-c3cncc(S(N)(=O)=O)c3)cnc2cn1)c1ccc(C#N)cc1]

The compound was synthesized using I-59 and an appropriate boronic acid/ester using a procedure similar Suzuki coupling protocol described in 228. M/Z=434.1 (M+1), r.t=1.43 mins.

### Synthesis of Compound 185

### N-(4-cyanophenyl)-3-{4-[methoxy(methyl)carbamoyl]phenyl}-N-methylimidazo[1,2-a]pyrazine-6-carboxamide

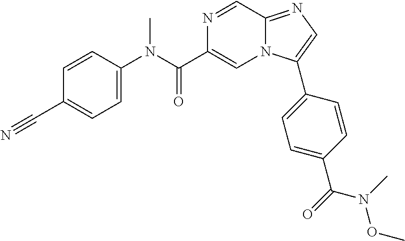 [SMILES: CON(C)C(=O)c1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. M/Z=441.1 (M+1), r.t=1.32 mins.

### Synthesis of Compound 186

### 3-[4-(aminomethyl)phenyl]-N-(4-cyanophenyl)-N-methylimidazo[1,2-a]pyrazine-6-carboxamide

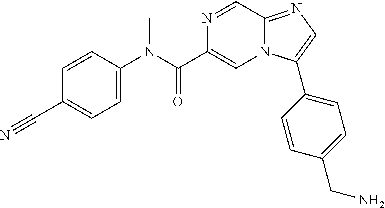 [SMILES: CN(C(=O)c1cn2c(-c3ccc(CN)cc3)cnc2cn1)c1ccc(C#N)cc1]

The compound was synthesized using I-59 and an appropriate boronic acid/ester using a procedure similar Suzuki coupling protocol described in 228. M/Z=382.1 (M+1), r.t=1.72 mins.

### Synthesis of Compound 187

### N-(4-cyanophenyl)-N-methyl-3-[6-(trifluoromethyl)pyridin-3-yl]imidazo[1,2-a]pyrazine-6-carboxamide

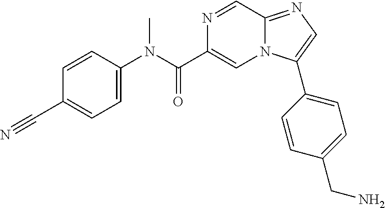 [SMILES: CN(C(=O)c1cn2c(-c3ccc(CN)cc3)cnc2cn1)c1ccc(C#N)cc1]

The compound was synthesized using I-59 and an appropriate boronic acid/ester using a procedure similar Suzuki coupling protocol described in 228. M/Z=423.1 (M+1), r.t=1.49 mins.

### Synthesis of Compound 188

### N-(4-cyanophenyl)-3-[4-(diethylcarbamoyl)phenyl]-N-methylimidazo[1,2-a]pyrazine-6-carboxamide

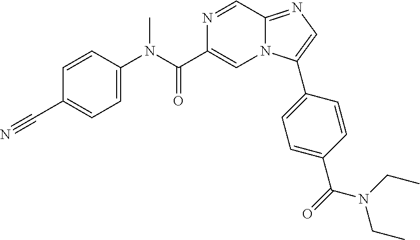 [SMILES: CCN(CC)C(=O)c1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. M/Z=453.1 (M+1), r.t=1.43 mins.

### Synthesis of Compound 189

### N-(4-cyanophenyl)-N-methyl-3-(1-methyl-1H-pyrazol-4-yl)imidazo[1,2-a]pyrazine-6-carboxamide

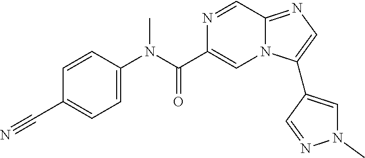 [SMILES: CN(C(=O)c1cn2c(-c3cnn(C)c3)cnc2cn1)c1ccc(C#N)cc1]

The compound was synthesized using I-59 and an appropriate boronic acid/ester using a procedure similar Suzuki coupling protocol described in 228. M/Z=358.2 (M+1), r.t=1.43 mins.

### Synthesis of Compound 190

### N-methyl-N-[(6-methylpyridin-2-yl)methyl]-3-[4-(trifluoromethyl)phenyl]imidazo[1,2-a]pyrazine-6-carboxamide

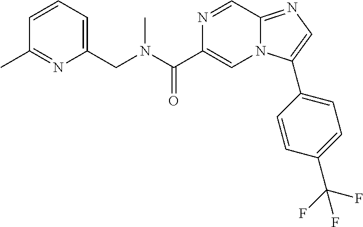 [SMILES: Cc1cccc(CN(C)C(=O)c2cn3c(-c4ccc(C(F)(F)F)cc4)cnc3cn2)n1]

The compound was synthesized using a procedure analogous to one use for synthesis of 72 (method II). MS m/z 426.1 (M+H)+; r.t.=1.285.

### Synthesis of Compound 191

### 3-(3-cyanophenyl)-N-(4-cyanophenyl)-N-methylimidazo[1,2-a]pyrazine-6-carboxamide

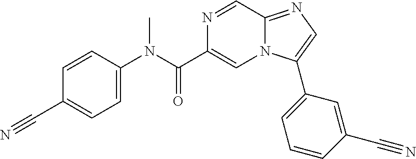 [SMILES: CN(C(=O)c1cn2c(-c3cccc(C#N)c3)cnc2cn1)c1ccc(C#N)cc1]

The compound was synthesized using I-59 and an appropriate boronic acid/ester using a procedure similar Suzuki coupling protocol described in 228. M/Z=379.1 (M+1), r.t=1.50 mins.

### Synthesis of Compound 192

### N-[(4-fluorophenyl)methyl]-N-methyl-3-[4-(trifluoromethyl)phenyl]imidazo[1,2-a]pyrazine-6-carboxamide

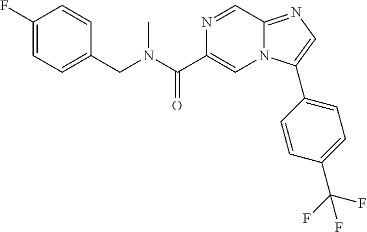 [SMILES: CN(Cc1ccc(F)cc1)C(=O)c1cn2c(-c3ccc(C(F)(F)F)cc3)cnc2cn1]

The compound was synthesized using I-62 and an appropriate boronic acid/ester using a procedure similar Suzuki coupling protocol described in 228. MS m/z 429.1 (M+H)+; r.t.=1.867.

### Synthesis of Compound 193

### N-(4-cyanophenyl)-N-methyl-3-[4-(pyrrolidin-2-yl)phenyl]imidazo[1,2-a]pyrazine-6-carboxamide

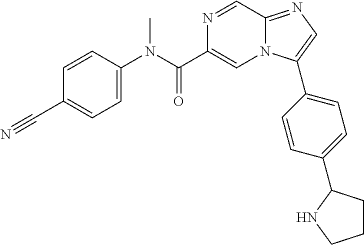 [SMILES: CN(C(=O)c1cn2c(-c3ccc(C4CCCN4)cc3)cnc2cn1)c1ccc(C#N)cc1]

The compound was synthesized using I-59 and an appropriate boronic acid/ester using a procedure similar Suzuki coupling protocol described in 212. MS m/z 423.1 (M+H)+; r.t.=1.350.

### Synthesis of Compound 194

### N-methyl-N-(pyridin-3-ylmethyl)-3-[4-(trifluoromethyl)phenyl]imidazo[1,2-a]pyrazine-6-carboxamide

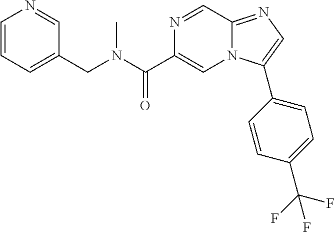 [SMILES: CN(Cc1cccnc1)C(=O)c1cn2c(-c3ccc(C(F)(F)F)cc3)cnc2cn1]

The compound was synthesized using a procedure analogous to one use for synthesis of 72 (method II). MS m/z 412.1 (M+H)+; r.t.=1.221.

### Synthesis of Compound 195

### N-(4-cyanophenyl)-N-methyl-3-(pyridin-3-yl)imidazo[1,2-a]pyrazine-6-carboxamide

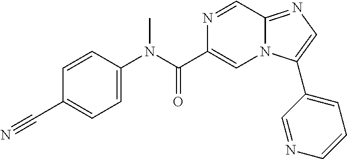 [SMILES: CN(C(=O)c1cn2c(-c3cccnc3)cnc2cn1)c1ccc(C#N)cc1]

The compound was synthesized using I-59 and an appropriate boronic acid/ester using a procedure similar Suzuki coupling protocol described in 228. M/Z=355.2 (M+1), r.t=1.31 mins.

### Synthesis of Compound 196

### 3-(5-chloropyridin-2-yl)-N-(4-cyanophenyl)-N-methylimidazo[1,2-a]pyrazine-6-carboxamide

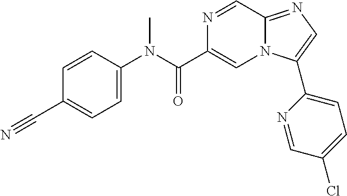 [SMILES: CN(C(=O)c1cn2c(-c3ccc(Cl)cn3)cnc2cn1)c1ccc(C#N)cc1]

The compound was synthesized using I-59 and an appropriate boronic acid/ester using a procedure similar Suzuki coupling protocol described in 228. M/Z=389.1 (M+1), r.t=1.92 mins.

### Synthesis of Compound 197

### N-(4-cyanophenyl)-3-[4-(dimethylsulfamoyl)phenyl]-N-methylimidazo[1,2-a]pyrazine-6-carboxamide

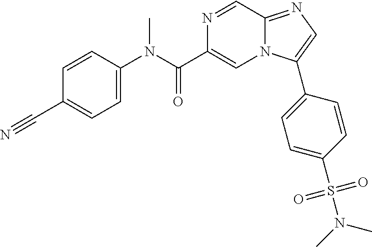 [SMILES: CN(C(=O)c1cn2c(-c3ccc(S(=O)(=O)N(C)C)cc3)cnc2cn1)c1ccc(C#N)cc1]

The compound was synthesized using I-59 and an appropriate boronic acid/ester using a procedure similar Suzuki coupling protocol described in 228. M/Z=461.1 (M+1), r.t=1.84 mins.

### Synthesis of Compound 198

### N-(4-cyanophenyl)-N-methyl-3-[2-(4-methylpiperidin-1-yl)-1,3-thiazol-4-yl]imidazo[1,2-a]pyrazine-6-carboxamide

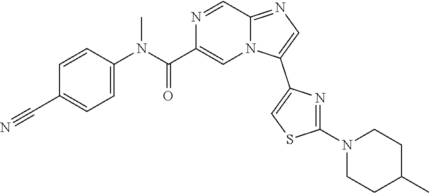 [SMILES: CC1CCN(c2nc(-c3cnc4cnc(C(=O)N(C)c5ccc(C#N)cc5)cn34)cs2)CC1]

The compound was synthesized using I-59 and an appropriate boronic acid/ester using a procedure similar Suzuki coupling protocol described in 228. M/Z=458.1 (M+1), r.t=2.38 mins.

### Synthesis of Compound 199

### N-(4-cyanophenyl)-N-methyl-3-(3-methylphenyl)imidazo[1,2-a]pyrazine-6-carboxamide

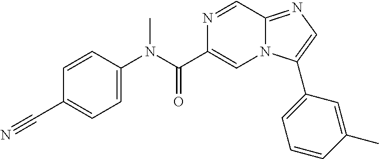 [SMILES: Cc1cccc(-c2cnc3cnc(C(=O)N(C)c4ccc(C#N)cc4)cn23)c1]

The compound was synthesized using I-59 and an appropriate boronic acid/ester using a procedure similar Suzuki coupling protocol described in 228. M/Z=368.1 (M+1), r.t=1.65 mins.

### Synthesis of Compound 200

### N-(4-cyanophenyl)-N-methyl-3-(1H-pyrazol-4-yl)imidazo[1,2-a]pyrazine-6-carboxamide

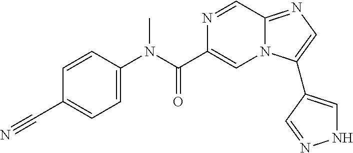 [SMILES: CN(C(=O)c1cn2c(-c3cn[nH]c3)cnc2cn1)c1ccc(C#N)cc1]

The compound was synthesized using I-59 and an appropriate boronic acid/ester using a procedure similar Suzuki coupling protocol described in 228. M/Z=344.2 (M+1), r.t=1.38 mins.

### Synthesis of Compound 201

### N-(4-cyanophenyl)-N-methyl-3-(1-methyl-2,3-dihydro-1H-indol-5-yl)imidazo[1,2-a]pyrazine-6-carboxamide

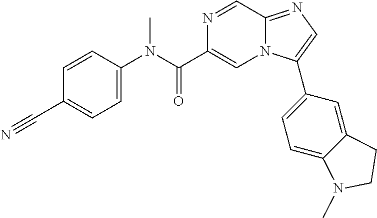 [SMILES: CN1CCc2cc(-c3cnc4cnc(C(=O)N(C)c5ccc(C#N)cc5)cn34)ccc21]

The compound was synthesized using I-59 and an appropriate boronic acid/ester using a procedure similar Suzuki coupling protocol described in 228. M/Z=409.1 (M+1), r.t=1.90 mins.

### Synthesis of Compound 202

### 3-(2,1,3-benzoxadiazol-5-yl)-N-(4-cyanophenyl)-N-methylimidazo[1,2-a]pyrazine-6-carboxamide

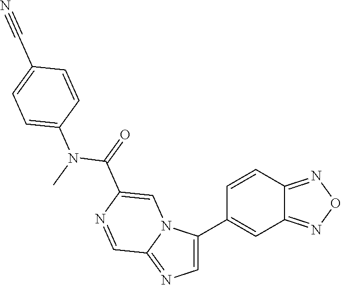 [SMILES: CN(C(=O)c1cn2c(-c3ccc4nonc4c3)cnc2cn1)c1ccc(C#N)cc1]

The compound was synthesized using I-59 and an appropriate boronic acid/ester using a procedure similar Suzuki coupling protocol described in 228. M/Z=396.1 (M+1), r.t=1.86 mins.

### Synthesis of Compound 203

### N-(4-cyanophenyl)-N-methyl-3-{3-methyl-3H-imidazo[4,5-b]pyridin-6-yl}imidazo[1,2-a]pyrazine-6-carboxamide

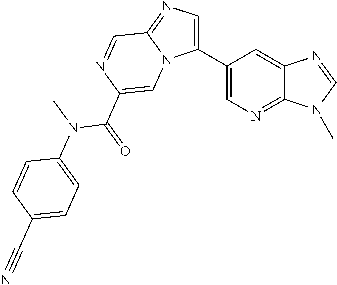 [SMILES: CN(C(=O)c1cn2c(-c3cnc4c(c3)ncn4C)cnc2cn1)c1ccc(C#N)cc1]

The compound was synthesized using I-59 and an appropriate boronic acid/ester using a procedure similar Suzuki coupling protocol described in 228. MS m/z 409.1 (M+H)+; r.t.=1.071.

### Synthesis of Compound 204

### N-(4-cyanophenyl)-N-methyl-3-(2-oxo-2,3-dihydro-1H-indol-5-yl)imidazo[1,2-a]pyrazine-6-carboxamide

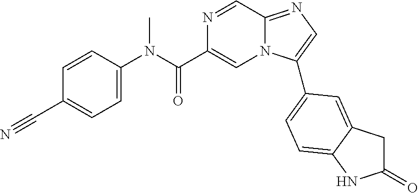 [SMILES: CN(C(=O)c1cn2c(-c3ccc4c(c3)CC(=O)N4)cnc2cn1)c1ccc(C#N)cc1]

The compound was synthesized using I-59 and an appropriate boronic acid/ester using a procedure similar Suzuki coupling protocol described in 228. M/Z=409.1 (M+1), r.t=1.50 mins.

### Synthesis of Compound 205

### N-(4-methanesulfonylphenyl)-N-methyl-3-(1-methyl-1H-indazol-5-yl)imidazo[1,2-a]pyrazine-6-carboxamide

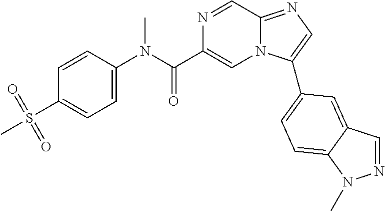 [SMILES: CN(C(=O)c1cn2c(-c3ccc4c(cnn4C)c3)cnc2cn1)c1ccc(S(C)(=O)=O)cc1]

The compound was synthesized using I-66 and an appropriate boronic acid/ester using a procedure similar Suzuki coupling protocol described in 228. MS m/z 461.1 (M+H)+; r.t.=1.254.

### Synthesis of Compound 206

### -methyl-N-{4-[3-(trifluoromethyl)-1H-pyrazol-1-yl]phenyl}-3-[4-(trifluoromethyl)phenyl]imidazo[1,2-a]pyrazine-6-carboxamide

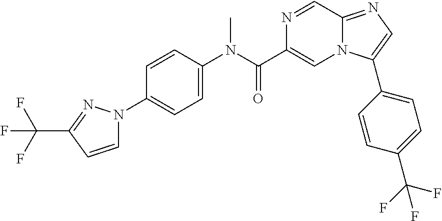 [SMILES: CN(C(=O)c1cn2c(-c3ccc(C(F)(F)F)cc3)cnc2cn1)c1ccc(-n2ccc(C(F)(F)F)n2)cc1]

The compound was synthesized using a procedure analogous to one use for synthesis of 72 (method II). MS m/z 531.1 (M+H)+; r.t.=1.867.

### Synthesis of Compound 207

### N-(4-cyanophenyl)-3-{4-[(dimethylamino)methyl]phenyl}-N-methylimidazo[1,2-a]pyrazine-6-carboxamide

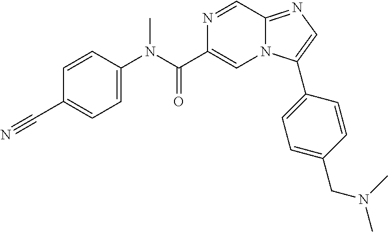 [SMILES: CN(C)Cc1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. M/Z=411.2 (M+1), r.t=1.18 mins.

### Synthesis of Compound 208

### N-(4-cyanophenyl)-N-methyl-3-[4-(1-methyl-1H-pyrazol-5-yl)phenyl]imidazo[1,2-a]pyrazine-6-carboxamide

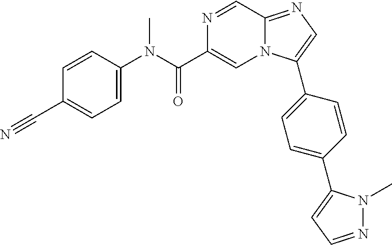 [SMILES: CN(C(=O)c1cn2c(-c3ccc(-c4ccnn4C)cc3)cnc2cn1)c1ccc(C#N)cc1]

The compound was synthesized using I-59 and I-27 using a procedure similar Suzuki coupling protocol described in 228. M/Z=434.1 (M+1), r.t=1.39 mins.

### Synthesis of Compound 209

### 3-[4-(difluoromethoxy)phenyl]-N-(4-methanesulfonylphenyl)-N-methylimidazo[1,2-a]pyrazine-6-carboxamide

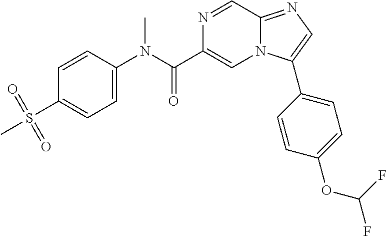 [SMILES: CN(C(=O)c1cn2c(-c3ccc(OC(F)F)cc3)cnc2cn1)c1ccc(S(C)(=O)=O)cc1]

The compound was synthesized using I-66 and an appropriate boronic acid/ester using a procedure similar Suzuki coupling protocol described in 228. MS m/z 473.0 (M+H)+; r.t.=1.469.

### Synthesis of Compound 210

### 4-[3-(4-carbamoylphenyl)imidazo[1,2-a]pyrazin-6-yl]benzamide

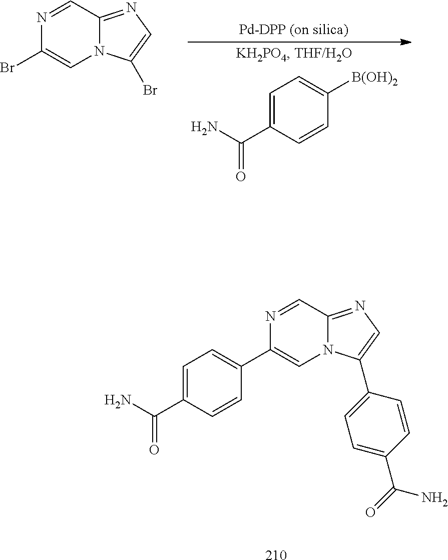 [SMILES: Brc1cn2c(Br)cnc2cn1 ; NC(=O)c1ccc(B(O)O)cc1 ; NC(=O)c1ccc(-c2cn3c(-c4ccc(C(N)=O)cc4)cnc3cn2)cc1 ; "210"]

Pd-DPP (on silica) (25 mg) was added to a solution of 3,6-dibromoimidazo[1,2-a]pyrazine (20 mg, 0.073 mmol), 4-carbamoylphenyl boronic acid (50 mg, 0.291 mmol), KH₂PO₄ (60 mg, 0.44 mmol), THF (1.0 mL) and water (0.5 mL). The reaction was heated in a microwave reactor at 150° C. for 45 minutes. After cooling to room temperature, the reaction was filtered to remove the catalyst. The reaction was purified by mass-trigger HPLC. NMR (400 MHz,) δ 9.30 (s, 1H), 9.04 (s, 1H), 8.34-7.83 (m, 11H), 7.49 (s, 1H), 7.42 (s, 1H). MS m/z 358.1 (M+H)+; r.t.=1.049.

### Synthesis of Compound 211

### N-(4-cyanophenyl)-N-methyl-3-[5-(2-oxopyrrolidin-1-yl)pyrazin-2-yl]imidazo[1,2-a]pyrazine-6-carboxamide

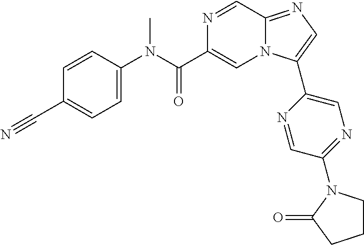 [SMILES: CN(C(=O)c1cn2c(-c3cnc(N4CCCC4=O)cn3)cnc2cn1)c1ccc(C#N)cc1]

The compound was synthesized using I-59 and an appropriate boronic acid/ester using a procedure similar Suzuki coupling protocol described in 228. M/Z=439.1 (M+1), r.t=1.39 mins.

### Synthesis of Compound 212

### 3-{4-[(2S)-2-amino-2-cyclohexylacetamido]phenyl}-N-(4-cyanophenyl)-N-methylimidazo[1,2-a]pyrazine-6-carboxamide

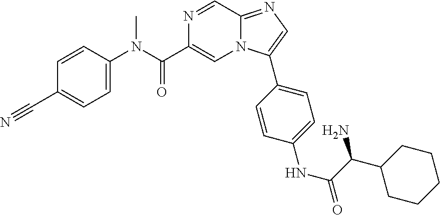 [SMILES: CN(C(=O)c1cn2c(-c3ccc(NC(=O)[C@@H](N)C4CCCCC4)cc3)cnc2cn1)c1ccc(C#N)cc1]

To a solution of 3-(4-aminophenyl)-N-(4-cyanophenyl)-N-methylimidazo[1,2-a]pyrazine-6-carboxamide 124 (30 mg, 0.081 mmols, 1 eq) in DCM was added the (S)-tert-butyl(2-amino-1-cyclohexyl-2-oxoethyl)carbamate (31 mg, 0.122 mmols, 1.5 eq), followed by HATU (46 mg, 0.121 mmols, 1.5 eq), and DIEA (26 mg, 0.20 mmols, 2.5 eq). The reaction mixture was stirred for 12 hrs and then removed all solvents and extracted with ethyl acetate (3×10 mL). The solvent was removed under reduce pressure. The product was treated with TFA in dichloromethane for 8 hours. The organics were concentrated and purified via preparative HPLC as a TFA salt. M/Z=508.23 (M+1), r.t=1.25 mins.

### Synthesis of Compound 213

### 4-[methyl({3-[4-(trifluoromethyl)phenyl]imidazo[1,2-a]pyrazin-6-yl}methyl)amino]benzonitrile

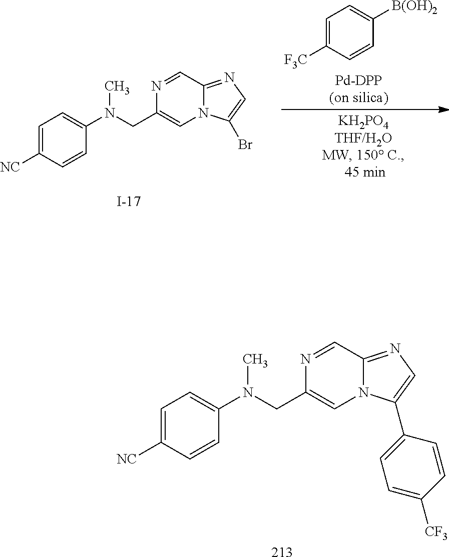

Pd-DPP (on silica) (25 mg) was added to a solution of bromoimidazolopyrazine (45 mg, 0.131 mmol), 4-trifluoromethylphenyl boronic acid (50 mg, 0.263 mmol), KH₂PO₄ (136 mg, 0.39 mmol), THF (3 mL) and water (1 mL). The reaction was heated in a microwave reactor at 150° C. for 45 minutes. After cooling to room temperature, the reaction was filtered to remove the catalyst. The reaction was purified by mass-trigger HPLC. ¹H NMR (400 MHz, DMSO) δ 9.14 (d, J=1.3, 1H), 8.72 (d, J=1.1, 1H), 8.21-8.13 (m, 1H), 7.98-7.90 (m, 4H), 7.53 (d, J=9.0, 2H), 6.95 (d, J=9.1, 2H), 4.78 (s, 2H), 3.15 (s, 3H); MS m/z 408.1 (M+H)+; r.t.=1.826.

### Synthesis of Compound 214

### N-(4-cyanophenyl)-3-[4-(difluoromethoxy)phenyl]-N-methylimidazo[1,2-a]pyrazine-6-carboxamide

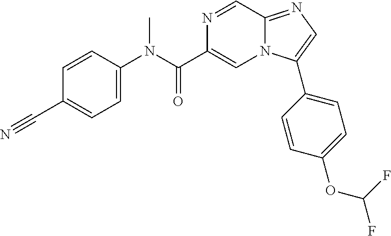 [SMILES: CN(C(=O)c1cn2c(-c3ccc(OC(F)F)cc3)cnc2cn1)c1ccc(C#N)cc1]

The compound was synthesized using I-59 and an appropriate boronic acid/ester using a procedure similar Suzuki coupling protocol described in 228. M/Z=420.1 (M+1), r.t=2.01 mins.

### Synthesis of Compound 215

### N-(4-bromophenyl)-N-methyl-3-[4-(trifluoromethyl)phenyl]imidazo[1,2-a]pyrazine-6-carboxamide

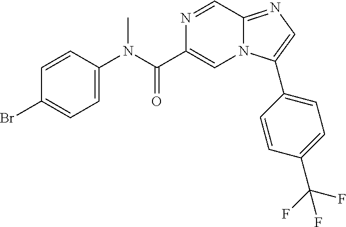 [SMILES: CN(C(=O)c1cn2c(-c3ccc(C(F)(F)F)cc3)cnc2cn1)c1ccc(Br)cc1]

The compound was synthesized using a procedure analogous to one use for synthesis of 72 (method II). MS m/z 477.0 (M+H)+; r.t.=2.310.

### Synthesis of Compound 216

### N-(6-chloropyridin-3-yl)-N-methyl-3-[4-(piperidin-1-yl)phenyl]imidazo[1,2-a]pyrazine-6-carboxamide

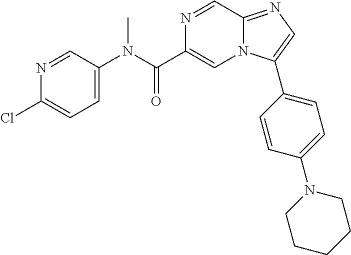 [SMILES: CN(C(=O)c1cn2c(-c3ccc(N4CCCCC4)cc3)cnc2cn1)c1ccc(Cl)nc1]

The compound was synthesized using I-57 and an appropriate boronic acid/ester using a procedure similar Suzuki coupling protocol described in 228. M/Z=447.2 (M+1), r.t=1.08 mins.

### Synthesis of Compound 217

### N-(4-chlorophenyl)-N-methyl-3-[4-(trifluoromethyl)phenyl]imidazo[1,2-a]pyrazine-6-carboxamide

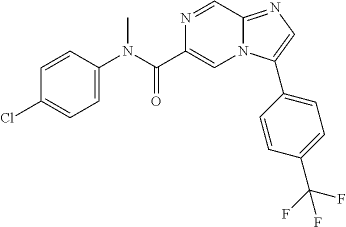 [SMILES: CN(C(=O)c1cn2c(-c3ccc(C(F)(F)F)cc3)cnc2cn1)c1ccc(Cl)cc1]

The compound was synthesized using I-11 and an appropriate boronic acid/ester using a procedure similar Suzuki coupling protocol described in 228. MS m/z 431.0 (M+H)+; r.t.=2.230.

### Synthesis of Compound 218

### 6-chloro-1-({3-[4-(trifluoromethyl)phenyl]imidazo[1,2-a]pyrazin-6-yl}carbonyl)-1,2,3,4-tetrahydroquinoline

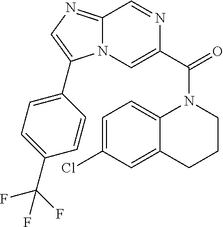 [SMILES: O=C(c1cn2c(-c3ccc(C(F)(F)F)cc3)cnc2cn1)N1CCCc2cc(Cl)ccc21]

The compound was synthesized using a procedure analogous to one use for synthesis of 72 (method II). MS m/z 457.0 (M+H)+; r.t.=2.210.

### Synthesis of Compound 219

### N-(4-chlorophenyl)-3-(4-methoxyphenyl)-N-methylimidazo[1,2-a]pyrazine-6-carboxamide

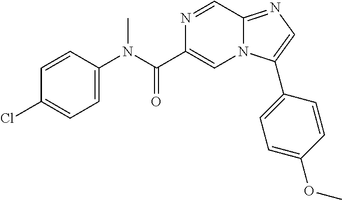 [SMILES: COc1ccc(-c2cnc3cnc(C(=O)N(C)c4ccc(Cl)cc4)cn23)cc1]

The compound was synthesized using I-11 and an appropriate boronic acid/ester using a procedure similar Suzuki coupling protocol described in 228. MS m/z 393.2 (M+H)+; r.t.=2.020.

### Synthesis of Compound 220

### N-(2-chloro-5-methylpyridin-3-yl)-N-methyl-3-[4-(trifluoromethyl)phenyl]imidazo[1,2-a]pyrazine-6-carboxamide

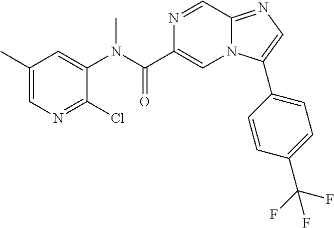 [SMILES: Cc1cnc(Cl)c(N(C)C(=O)c2cn3c(-c4ccc(C(F)(F)F)cc4)cnc3cn2)c1]

The compound was synthesized using a procedure analogous to one use for synthesis of 72 (method II) using I-50. MS m/z 446.1 (M+H)+; r.t.=1.707.

### Synthesis of Compound 221

### N-(3-fluoropyridin-2-yl)-N-methyl-3-[4-(trifluoromethyl)phenyl]imidazo[1,2-a]pyrazine-6-carboxamide

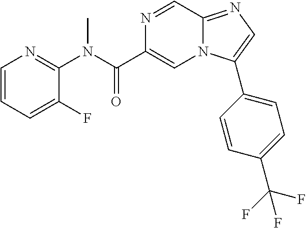 [SMILES: CN(C(=O)c1cn2c(-c3ccc(C(F)(F)F)cc3)cnc2cn1)c1ncccc1F]

The compound was synthesized using a procedure analogous to one use for synthesis of 72 (method II). MS m/z 416.1 (M+H)+; r.t.=1.625.

### Synthesis of Compound 222

### 4-(6-{[(4-fluorophenyl)(methyl)amino]methyl}imidazo[1,2-a]pyrazin-3-yl)benzamide

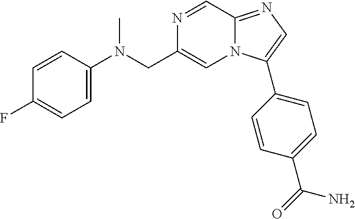 [SMILES: CN(Cc1cn2c(-c3ccc(C(N)=O)cc3)cnc2cn1)c1ccc(F)cc1]

The compound was synthesized using I-62 and an appropriate boronic acid/ester using a procedure similar Suzuki coupling protocol described in 228. ¹H NMR (400 MHz, DMSO) δ 9.14 (d, J=1.3, 1H), 8.47 (s, 1H), 8.12 (s, 2H), 8.04 (d, J=8.4, 2H), 7.72 (d, J=8.4, 2H), 7.51 (s, 1H), 7.00 (t, J=8.9, 2H), 6.84 (dd, J=4.4, 9.2, 2H), 4.65 (s, 2H), 3.03 (s, 3H); MS m/z 376.1 (M+H)+; r.t.=1.247.

### Synthesis of Compound 223

### 4-(6-{[(4-cyanophenyl)(methyl)amino]methyl}imidazo[1,2-a]pyrazin-3-yl)benzamide

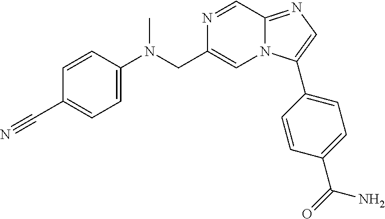 [SMILES: CN(Cc1cn2c(-c3ccc(C(N)=O)cc3)cnc2cn1)c1ccc(C#N)cc1]

The compound was synthesized using I-15 and an appropriate boronic acid/ester using a procedure similar Suzuki coupling protocol described in 228. 1H NMR (400 MHz, MeOD) δ 9.08 (d, J=1.3 Hz, 1H), 8.31 (d, J=1.0 Hz, 2H), 8.21-7.97 (m, 3H), 7.79-7.61 (m, 2H), 7.62-7.41 (m, 2H), 7.13-6.75 (m, 3H), 4.82 (s, 2H), 3.21 (s, 3H).

### Synthesis of Compound 224

### N-(6-chloropyridin-3-yl)-N-methyl-3-[4-(1H-pyrazol-5-yl)phenyl]imidazo[1,2-a]pyrazine-6-carboxamide

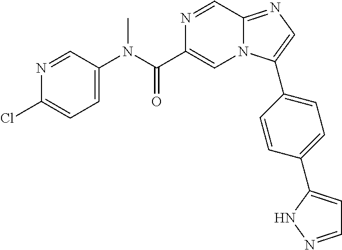 [SMILES: CN(C(=O)c1cn2c(-c3ccc(-c4ccn[nH]4)cc3)cnc2cn1)c1ccc(Cl)nc1]

The compound was synthesized using I-57 and an appropriate boronic acid/ester using a procedure similar Suzuki coupling protocol described in 228. MS m/z 430.1 (M+H)+; r.t.=1.334.

### Synthesis of Compound 225

### methyl N-[4-(6-{[(4-cyanophenyl)(methyl)amino]methyl}imidazo[1,2-a]pyrazin-3-yl)phenyl]carbamat

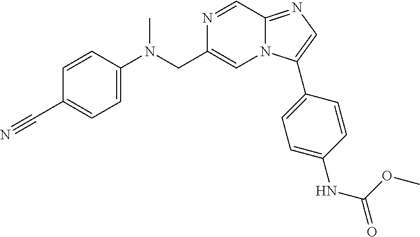 [SMILES: COC(=O)Nc1ccc(-c2cnc3cnc(CN(C)c4ccc(C#N)cc4)cn23)cc1]

The compound was synthesized using I-15 and an appropriate boronic acid/ester using a procedure similar Suzuki coupling protocol described in 228. MS m/z 413.2 (M+H)+; r.t.=1.510.

### Synthesis of Compound 226

### N-[5-(6-{[(4-cyanophenyl)(methyl)amino]methyl}imidazo[1,2-a]pyrazin-3-yl)pyridin-2-yl]acetamide

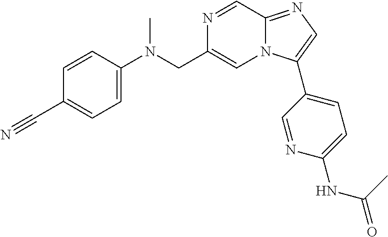 [SMILES: CC(=O)Nc1ccc(-c2cnc3cnc(CN(C)c4ccc(C#N)cc4)cn23)cn1]

The compound was synthesized using I-15 and an appropriate boronic acid/ester using a procedure similar Suzuki coupling protocol described in 228. ¹H NMR (400 MHz, DMSO) δ 10.76 (s, 1H), 9.11 (s, 1H), 8.64 (s, 2H), 8.28 (d, J=8.7, 1H), 8.12 (d, J=8.7, 1H), 8.09 (s, 1H), 7.51 (d, J=9.0, 2H), 6.95 (d, J=9.0, 2H), 4.76 (s, 2H), 3.14 (s, 3H), 2.15 (s, 3H); MS m/z 398.2 (M+H)+; r.t.=1.312.

### Synthesis of Compound 227

### 3-[4-(5-amino-1,3,4-oxadiazol-2-yl)phenyl]-N-(4-fluorophenyl)-N-methylimidazo[1,2-a]pyrazine-6-carboxamide

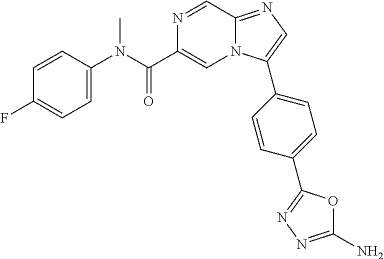 [SMILES: CN(C(=O)c1cn2c(-c3ccc(-c4nnc(N)o4)cc3)cnc2cn1)c1ccc(F)cc1]

The compound was synthesized using I-64 and an appropriate boronic acid/ester using a procedure similar Suzuki coupling protocol described in 228. 1H NMR (400 MHz, MeOD) δ 8.82-8.52 (m, 2H), 7.99 (m, 3H), 7.65 (m, 2H), 7.18 (m, 2H), 6.96 (m, 2H), 3.40 (s, 3H). M/Z=430.2 (M+1), r.t=1.21 mins.

### Synthesis of Compound 228

### N-(4-chlorophenyl)-N-methyl-3-[4-(methylcarbamoyl)phenyl]imidazo[1,2-a]pyrazine-6-carboxamide

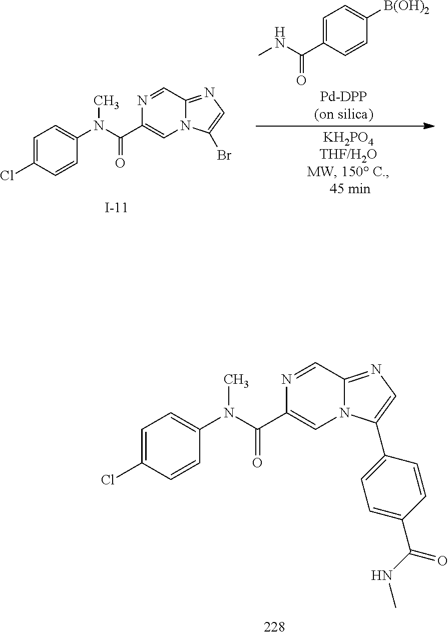

Pd-DPP (palladium diphenylphosphine supported on silica, Silicycle) (25 mg) was added to a solution of 3-bromo-N-(4-chlorophenyl)-N-methylimidazo[1,2-a]pyrazine-6-carboxamide (40 mg, 0.11 mmol), (4-(methylcarbamoyl)phenyl)boronic acid (50 mg, 0.28 mmol), K₂HPO₄ (50 mg, 0.36 mmol), THF (2.0 mL) and water (1.0 mL). The reaction was heated in a microwave reactor at 150° C. for 45 minutes. After cooling to room temperature, the reaction was concentrated in vacuo. The crude material was purified by flash chromatography (silica, 0-15% methanol/chloroform). ¹H NMR (400 MHz, DMSO) δ 8.86 (s, 1H), 8.81 (d, J=1.3, 1H), 8.62 (d, J=4.6, 1H), 8.19 (s, 1H), 8.05 (d, J=8.4, 2H), 7.79 (d, J=8.4, 2H), 7.35 (d, J=8.8, 2H), 7.30 (d, J=8.8, 2H), 3.42 (s, 3H), 2.84 (d, J=4.5, 3H). ¹H NMR (400 MHz, DMSO) δ 8.86 (s, 1H), 8.81 (d, J=1.3, 1H), 8.62 (d, J=4.6, 1H), 8.19 (s, 1H), 8.05 (d, J=8.4, 2H), 7.79 (d, J=8.4, 2H), 7.35 (d, J=8.8, 2H), 7.30 (d, J=8.8, 2H), 3.42 (s, 3H), 2.84 (d, J=4.5, 3H); MS m/z 420.2 (M+H)+; r.t.=0.98.

### Synthesis of Compound 229

### 4-{6-[(6-fluoro-2,2-dimethyl-1,2,3,4-tetrahydroquinolin-1-yl)carbonyl]imidazo[1,2-a]pyrazin-3-yl}benzamide

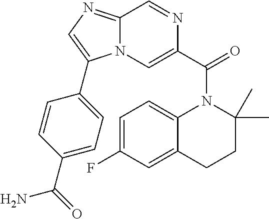 [SMILES: CC1(C)CCc2cc(F)ccc2N1C(=O)c1cn2c(-c3ccc(C(N)=O)cc3)cnc2cn1]

The compound was synthesized using I-71 and an appropriate boronic acid/ester using a procedure similar Suzuki coupling protocol described in 228. 1H NMR (300 MHz, DMSO-d₆) ppm 8.82 (d, J=9.67 Hz, 2H) 8.20 (s, 1H) 8.18 (br. s., 1H) 8.13 (d, J=8.50 Hz, 2H) 7.83 (d, J=8.50 Hz, 2H) 7.54 (br. s., 1H) 7.14 (dd, 1H) 6.80 (dd, J=8.79, 4.98 Hz, 1H) 6.65 (td, 1H) 2.76 (br. s., 2H) 1.83 (br. s., 2H) 1.67 (s, 6H); MS (ESI) for C₂₅H₂₂FN₅O₂ MS m/z: 444 (M+H⁺).

### Synthesis of Compound 230

### 6-fluoro-2,2-dimethyl-1-({3-[4-(trifluoromethyl)phenyl]imidazo[1,2-a]pyrazin-6-yl}carbonyl)-1,2,3,4-tetrahydroquinoline

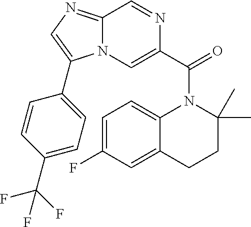 [SMILES: CC1(C)CCc2cc(F)ccc2N1C(=O)c1cn2c(-c3ccc(C(F)(F)F)cc3)cnc2cn1]

The compound was synthesized using I-71 and an appropriate boronic acid/ester using a procedure similar Suzuki coupling protocol described in 228. ¹H NMR (400 MHz, CD₃OD): δ 8.73 (s, 1H), 8.71 (s, 1H), 8.07 (s, 1H), 7.89 (d, J=8.34 Hz, 2H), 7.80 (d, J=8.34 Hz, 2H), 7.02-6.99 (m, 1H), 6.67-6.63 (m, 1H), 6.56-6.51 (m, 1H), 2.77 (t, J=4.83 Hz, 2H), 1.85 (bs, 2H), 1.70 (s, 6H). MS m/z: 469.12 (M+H).

### Synthesis of Compound 231

### 6-chloro-N-methyl-N-({3-[4-(trifluoromethyl)phenyl]imidazo[1,2-a]pyrazin-6-yl}methyl)pyridin-3-amine

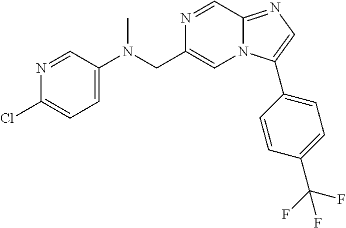 [SMILES: CN(Cc1cn2c(-c3ccc(C(F)(F)F)cc3)cnc2cn1)c1ccc(Cl)nc1]

The compound was synthesized using I-64 and an appropriate boronic acid/ester using a procedure similar Suzuki coupling protocol described in 228. MS m/z 418.1 (M+H)+; r.t.=1.740.

### Synthesis of Compound 232

### 3-(4-cyanophenyl)-N-(4-methanesulfonylphenyl)-N-methylimidazo[1,2-a]pyrazine-6-carboxamide

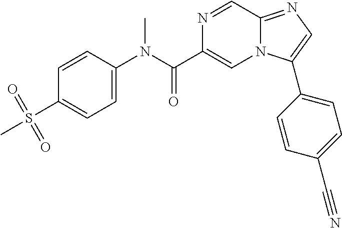 [SMILES: CN(C(=O)c1cn2c(-c3ccc(C#N)cc3)cnc2cn1)c1ccc(S(C)(=O)=O)cc1]

The compound was synthesized using I-66 and an appropriate boronic acid/ester using a procedure similar Suzuki coupling protocol described in 228. MS m/z 432.1 (M+H)+; r.t.=1.560.

### Synthesis of Compound 233

### 4-(6-{[methyl(5-methylpyridin-2-yl)amino]methyl}imidazo[1,2-a]pyrazin-3-yl)benzamide

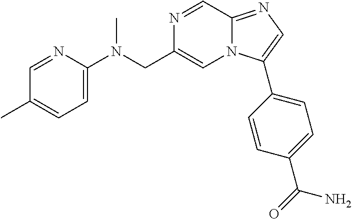 [SMILES: Cc1ccc(N(C)Cc2cn3c(-c4ccc(C(N)=O)cc4)cnc3cn2)nc1]

The compound was synthesized using I-77 and an appropriate boronic acid/ester using a procedure similar Suzuki coupling protocol described in 228. MS m/z 373.2 (M+H)+; r.t.=0.91.

### Synthesis of Compound 234

### 6-fluoro-1-({3-[4-(trifluoromethyl)phenyl]imidazo[1,2-a]pyrazin-6-yl}methyl)-1,2,3,4-tetrahydroquinoline

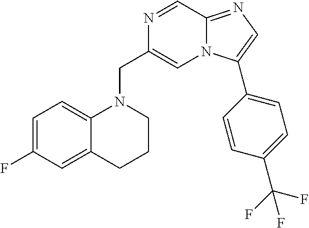 [SMILES: Fc1ccc2c(c1)CCCN2Cc1cn2c(-c3ccc(C(F)(F)F)cc3)cnc2cn1]

The compound was synthesized using a procedure analogous to one use for synthesis of 5. H-NMR (400 MHz, CDCl₃): 9.17 (d, J=0.98 Hz, 1H), 8.06 (d, J=1.45 Hz, 1H) 7.91 (s, 1H), 7.72 (d, J=8.36 Hz, 2H), 7.52 (d, J=8.3 Hz, 2H), 6.69-6.78 (m, 2H), 6.43-6.46 (m, 1H), 4.62 (s, 2H), 3.42 (t, J=5.85 Hz, 2H), 2.79 (t, J=5.85 Hz, 2H), 1.71-2.03 (m, 2H). MS m/z 427.2 (M+H).

### Synthesis of Compound 235

### N,5-dimethyl-N-({3-[4-(trifluoromethyl)phenyl]imidazo[1,2-a]pyrazin-6-yl}methyl)pyridin-3-amine

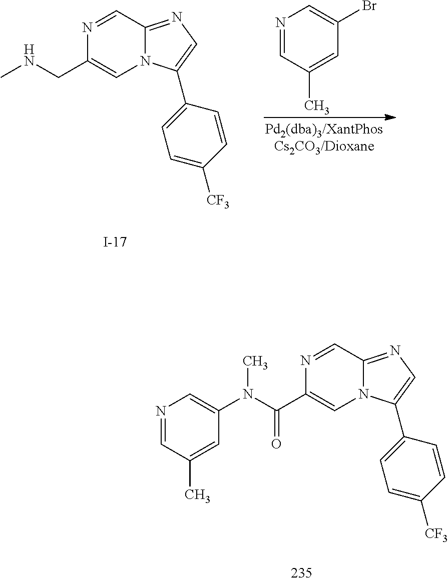

A vial containing the N-methylamine I-17 (17 mg, 0.057 mmol), bromopyridine (10 mg, 0.057 mmol), cesium carbonate (37 mg, 0.11 mmol), and dioxane (1.0 mL) was degassed under vacuum. Pd₂(dba)₃ (3.0 mg, 0.0030 mmol) and XantPhos (4.0 mg, 0.0060 mmol) were added. The vial was evacuated until gas evolution ceased. The evacuated vial was heated to 100° C. for 4 hours. After cooling to room temperature, the reaction was diluted with dichloromethane (1 mL). The mixture was filtered, and the filtrate concentrated under reduced pressure. The mixture was purified by mass-trigger HPLC. MS m/z 398.2 (M+H)+; r.t.=1.373.

### Synthesis of Compound 236

### N-(4-cyanophenyl)-3-(6-acetamidopyridin-3-yl)-N-methylimidazo[1,2-a]pyrazine-6-carboxamide

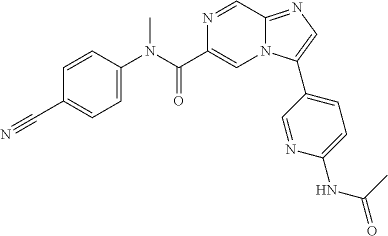 [SMILES: CC(=O)Nc1ccc(-c2cnc3cnc(C(=O)N(C)c4ccc(C#N)cc4)cn23)cn1]

The compound was synthesized using I-59 and an appropriate boronic acid/ester using a procedure similar Suzuki coupling protocol described in 228. M/Z=412.1 (M+1), r.t=1.39 mins.

### Synthesis of Compound 237

### N-(6-chloropyridin-3-yl)-3-[4-(1,1-difluoroethyl)phenyl]-N-methylimidazo[1,2-a]pyrazine-6-carboxamide

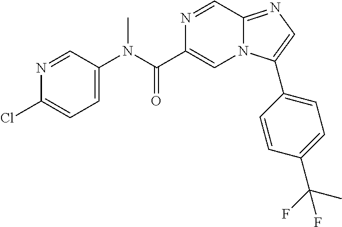 [SMILES: CN(C(=O)c1cn2c(-c3ccc(C(C)(F)F)cc3)cnc2cn1)c1ccc(Cl)nc1]

The compound was synthesized using I-57 and I-25 using a procedure similar Suzuki coupling protocol described in 228. M/Z=428.1 (M+1), r.t=1.61 mins.

### Synthesis of Compound 238

### N-(4-cyanophenyl)-N-(2-methoxyethyl)-3-[4-(trifluoromethyl)phenyl]imidazo[1,2-a]pyrazine-6-carboxamide

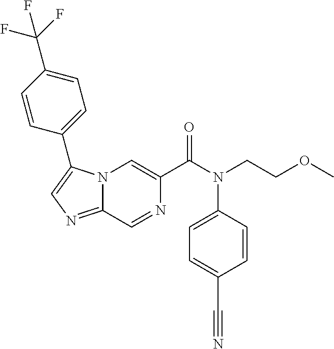 [SMILES: COCCN(C(=O)c1cn2c(-c3ccc(C(F)(F)F)cc3)cnc2cn1)c1ccc(C#N)cc1]

The compound was synthesized using a procedure analogous to one use for synthesis of 72 (method II) using intermediate I-30. MS m/z 466.2 (M+H)+; r.t.=1.847.

### Synthesis of Compound 239

### N-methyl-3-[4-(trifluoromethyl)phenyl]-N-[6-(trifluoromethyl)pyridin-3-yl]imidazo[1,2-a]pyrazine-6-carboxamide

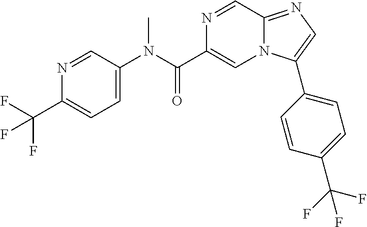 [SMILES: CN(C(=O)c1cn2c(-c3ccc(C(F)(F)F)cc3)cnc2cn1)c1ccc(C(F)(F)F)nc1]

The compound was synthesized using a procedure analogous to one use for synthesis of 72 (method II), using I-42. MS m/z 466.0 (M+H)+; r.t.=1.863.

### Synthesis of Compound 240

### N-(4-cyanophenyl)-N-methyl-3-[4-(1,3,4-oxadiazol-2-yl)phenyl]imidazo[1,2-a]pyrazine-6-carboxamide

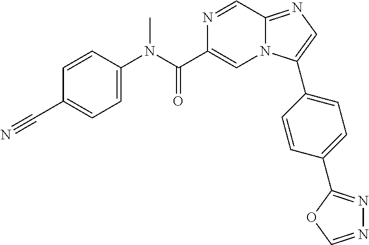 [SMILES: CN(C(=O)c1cn2c(-c3ccc(-c4nnco4)cc3)cnc2cn1)c1ccc(C#N)cc1]

The compound was synthesized using I-59 and an appropriate boronic acid/ester using a procedure similar Suzuki coupling protocol described in 228. M/Z=422.1 (M+1), r.t=1.26 mins.

### Synthesis of Compound 241

### N-({3-[4-(difluoromethyl)phenyl]imidazo[1,2-a]pyrazin-6-yl}methyl)-N,5-dimethylpyridin-2-amine

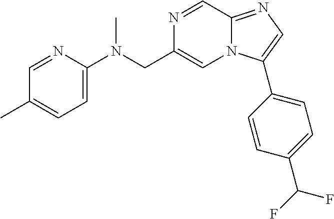 [SMILES: Cc1ccc(N(C)Cc2cn3c(-c4ccc(C(F)F)cc4)cnc3cn2)nc1]

The compound was synthesized using I-77 and an appropriate boronic acid/ester using a procedure similar Suzuki coupling protocol described in 228. MS m/z 380.2 (M+H)+; r.t.=1.280.

### Synthesis of Compound 242

### N-[5-(6-{[methyl(5-methylpyridin-2-yl)amino]methyl}imidazo[1,2-a]pyrazin-3-yl)pyridin-2-yl]acetamide

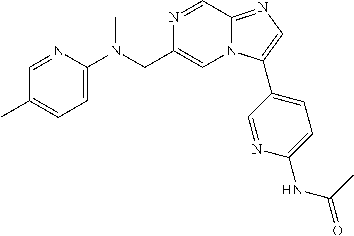 [SMILES: CC(=O)Nc1ccc(-c2cnc3cnc(CN(C)c4ccc(C)cn4)cn23)cn1]

The compound was synthesized using I-77 and an appropriate boronic acid/ester using a procedure similar Suzuki coupling protocol described in 228. MS m/z 388.2 (M+H)+; r.t.=0.939.

### Synthesis of Compound 243

### N-(4-cyanophenyl)-N-methyl-3-{1H-pyrrolo[2,3-b]pyridin-5-yl}imidazo[1,2-a]pyrazine-6-carboxamide

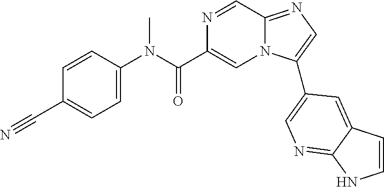 [SMILES: CN(C(=O)c1cn2c(-c3cnc4[nH]ccc4c3)cnc2cn1)c1ccc(C#N)cc1]

The compound was synthesized using I-59 and an appropriate boronic acid/ester using a procedure similar Suzuki coupling protocol described in 228. MS m/z 394.1 (M+H)+; r.t.=1.195.

### Synthesis of Compound 244

### N-(4-fluorophenyl)-N-methyl-3-[4-(methylcarbamoyl)phenyl]imidazo[1,2-a]pyrazine-6-carboxamide

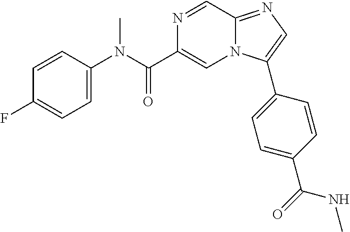 [SMILES: CNC(=O)c1ccc(-c2cnc3cnc(C(=O)N(C)c4ccc(F)cc4)cn23)cc1]

The compound was synthesized using I-64 and an appropriate boronic acid/ester using a procedure similar Suzuki coupling protocol described in 228. MS m/z 404.1 (M+H)+; r.t.=1.213.

### Synthesis of Compound 245

### methyl N-(4-{6-[(6-chloropyridin-3-yl)(methyl)carbamoyl]imidazo[1,2-a]pyrazin-3-yl}phenyl)carbamate

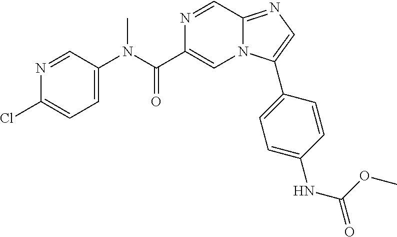 [SMILES: COC(=O)Nc1ccc(-c2cnc3cnc(C(=O)N(C)c4ccc(Cl)nc4)cn23)cc1]

The compound was synthesized using I-57 and an appropriate boronic acid/ester using a procedure similar Suzuki coupling protocol described in 228. ¹H NMR (400 MHz, MeOD) δ 8.93 (s, 1H), 8.72 (s, 1H), 8.27 (s, 1H), 7.99 (s, 1H), 7.78 (d, J=8.2 Hz, 1H), 7.72 (d, J=8.4 Hz, 2H), 7.61 (d, J=8.6 Hz, 2H), 7.45 (d, J=8.5 Hz, 1H), 3.79 (s, 3H), 3.54 (s, 3H), ESI-MS m/z 437.1 [M+1]+. RT: 1.31 min.

### Synthesis of Compound 246

### N-(4-cyanophenyl)-N-methyl-3-[6-(methylamino)pyridin-3-yl]imidazo[1,2-a]pyrazine-6-carboxamide

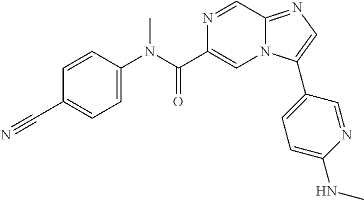 [SMILES: CNc1ccc(-c2cnc3cnc(C(=O)N(C)c4ccc(C#N)cc4)cn23)cn1]

The compound was synthesized using I-59 and an appropriate boronic acid/ester using a procedure similar Suzuki coupling protocol described in 228. M/Z=384.1 (M+1), r.t=0.94 mins.

### Synthesis of Compound 247

### 3-[4-(2-aminoacetamido)phenyl]-N-(4-cyanophenyl)-N-methylimidazo[1,2-a]pyrazine-6-carboxamide

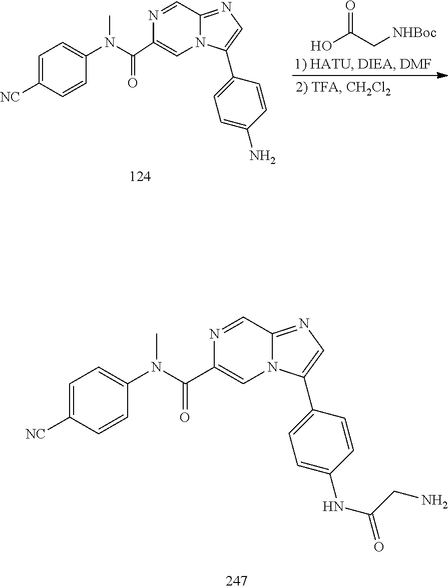 [SMILES: CN(C(=O)c1cn2c(-c3ccc(N)cc3)cnc2cn1)c1ccc(C#N)cc1 ; CC(C)(C)OC(=O)NCC(=O)O ; "124" ; CN(C(=O)c1cn2c(-c3ccc(NC(=O)CN)cc3)cnc2cn1)c1ccc(C#N)cc1 ; "247"]

To a stirred solution of 124 whose synthesis is previously described (100 mg, 0.27 mmol, 1.0 eq.) and 2-(tert-butoxycarbonyl)amino)acetic acid (52.3 mg, 0.29 mmol, 1.1 eq.) in 6 mL of DMF were added HATU (124 mg, 0.32 mmol, 1.2 eq.) and DIEA (162 μL, 0.32 mmol, 1.2 eq). The reaction mixture was stirred at room temperature for 8 hours. HPLC/MS test showed that desired product was formed. The product was partitioned in water/Ethyl acetate and purified by column chromatography to yield the desired product (50 mg, 35%). The Boc-derivative was carried to the next step. To a stirred solution of Boc-derivative (50 mg) in 2 mL of dichloromethane was added trifluoroacetic acid (2 ml) was stirred for 3 hours. LCMS indicated that the reaction was complete. The reaction was concentrated and purified by reverse phase HPLC to yield desired product. The product was characterized by reverse phase HPLC using method B. (ES, m/z): [M+H⁺] 426.2. Retention time=0.83 mins. M/Z=426.2 (M+1), r.t=0.83 mins.

### Synthesis of Compound 248

### 3-[4-(5-amino-1,3,4-oxadiazol-2-yl)phenyl]-N-(4-cyanophenyl)-N-methylimidazo[1,2-a]pyrazine-6-carboxamide

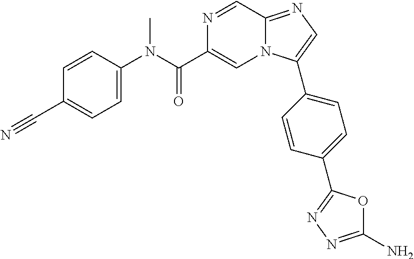 [SMILES: CN(C(=O)c1cn2c(-c3ccc(-c4nnc(N)o4)cc3)cnc2cn1)c1ccc(C#N)cc1]

The compound was synthesized using I-59 and I-24 using a procedure similar Suzuki coupling protocol described in 228. M/Z=437.1 (M+1), rt=1.19 mins.

### Synthesis of Compound 249

### N-methyl-N-(pyridin-4-yl)-3-[4-(trifluoromethyl)phenyl]imidazo[1,2-a]pyrazine-6-carboxamide

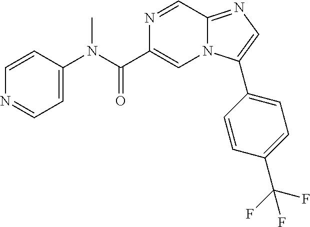 [SMILES: CN(C(=O)c1cn2c(-c3ccc(C(F)(F)F)cc3)cnc2cn1)c1ccncc1]

The compound was synthesized using a procedure analogous to one use for synthesis of 72 (method II). MS m/z 398.1 (M+H)+; r.t.=1.278.

### Synthesis of Compound 250

### N-(2-methoxy-5-methylpyridin-3-yl)-N-methyl-3-[4-(trifluoromethyl)phenyl]imidazo[1,2-a]pyrazine-6-carboxamide

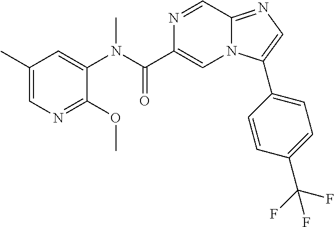 [SMILES: COc1ncc(C)cc1N(C)C(=O)c1cn2c(-c3ccc(C(F)(F)F)cc3)cnc2cn1]

The compound was synthesized using a procedure analogous to one use for synthesis of 72 (method II). M/Z=442.14 (M+1), r.t=1.48 mins.

### Synthesis of Compound 251

### N-methyl-N-(5-methylpyridin-3-yl)-3-[4-(trifluoromethyl)phenyl]imidazo[1,2-a]pyrazine-6-carboxamide

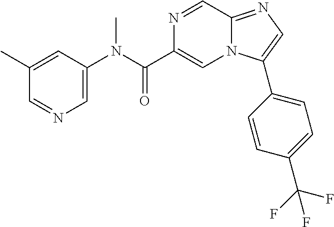 [SMILES: Cc1cncc(N(C)C(=O)c2cn3c(-c4ccc(C(F)(F)F)cc4)cnc3cn2)c1]

The compound was synthesized using a procedure analogous to one use for synthesis of 72 (method II). ¹H NMR (400 MHz, CDCl3) δ 8.79 (s, J=1.3 Hz, 1H), 8.63 (s, 1H), 8.22 (s, 1H), 8.02 (s, 1H), 7.88 (s, 1H), 7.77 (d, J=8.2 Hz, 2H), 7.63 (d, J=8.2 Hz, 2H), 7.37 (s, 1H), 3.47 (s, 3H), 2.30 (s, 3H). ESI-MS m/z 412 [M+1]. RT: 1.36 min.

### Synthesis of Compound 252

### N-(2-methoxy-5-methylpyridin-3-yl)-N-methyl-3-[4-(trifluoromethyl)phenyl]imidazo[1,2-a]pyrazine-6-carboxamide

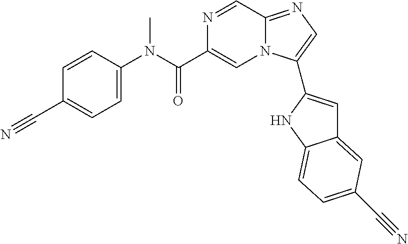 [SMILES: CN(C(=O)c1cn2c(-c3cc4cc(C#N)ccc4[nH]3)cnc2cn1)c1ccc(C#N)cc1]

The compound was synthesized using I-59 and an appropriate boronic acid/ester using a procedure similar Suzuki coupling protocol described in 228. M/Z=418.1 (M+1), r.t=1.49 mins.

### Synthesis of Compound 253

### ethyl 6-[(6-chloropyridin-3-yl)(methyl)carbamoyl]imidazo[1,2-a]pyrazine-3-carboxylate

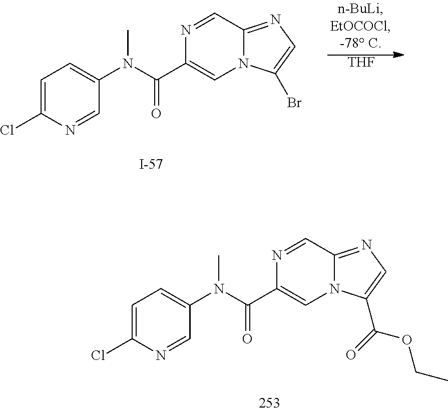

To a solution of 3-bromo-N-(4-chlorophenyl)-N-methylimidazo[1,2-a]pyrazine-6-carboxamide (I-57) (0.09 g, 0.245 mmol, 1 eq), in THF was added n-BuLi (0.245 mmol, 1 eq). The reaction mixture was stirred at −78° C. for ½ hr and at this point the ethyl chloroformate (0.026 g, 0.245 mmol) was added. The reaction mixture was stirred at −78° C. for 4 more hours and then slowly warmed up to room temperature. Quenched with ice-water and extracted the organics with ethyl acetate (3×10 mL). The organics were combined and dried over sodium sulfate and then purified using column chromatography. Yield=35 mg (0.10 g mmols, 43%) of the desired product. ¹H NMR (400 MHz, MeOD) δ 9.69-9.54 (m, 1H), 8.27 (s, 1H), 7.82-7.66 (m, 1H), 7.36 (d, J=8.6 Hz, 1H), 4.08 (q, J=7.1 Hz, 1H), 3.45 (s, 3H), 2.60 (s, 3H), 1.17 (t, J=7.1 Hz, 3H), ESI-MS m/z 360 [M+H]+. RT: 1.28 min.

### Synthesis of Compound 254

### ethyl N-(4-{6-[(4-cyanophenyl)(methyl)carbamoyl]imidazo[1,2-a]pyrazin-3-yl}phenyl)carbamate

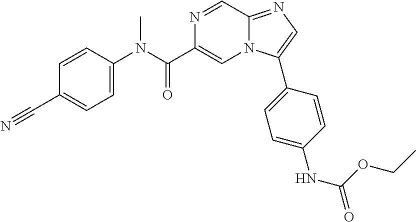 [SMILES: CCOC(=O)Nc1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. M/Z=441.2 (M+1), r.t=1.30 mins.

### Synthesis of Compound 255

### 3-(4-{[(1S,2R)-2-aminocyclopentane]amido}phenyl)-N-(4-cyanophenyl)-N-methylimidazo[1,2-a]pyrazine-6-carboxamide

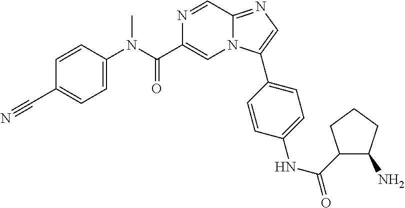 [SMILES: CN(C(=O)c1cn2c(-c3ccc(NC(=O)C4CCC[C@H]4N)cc3)cnc2cn1)c1ccc(C#N)cc1]

The compound was synthesized using a procedure described in 212. M/Z=480.2 (M+1), r.t=0.90 mins.

### Synthesis of Compound 256

### 3-{4-[(3S)-3-amino-4-methylpentanamido]phenyl}-N-(4-cyanophenyl)-N-methylimidazo[1,2-a]pyrazine-6-carboxamide

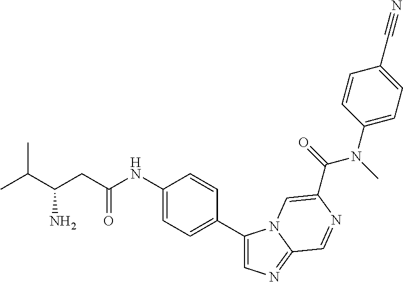 [SMILES: CC(C)[C@@H](N)CC(=O)Nc1ccc(-c2cnc3cnc(C(=O)N(C)c4ccc(C#N)cc4)cn23)cc1]

The compound was synthesized using a procedure described in 212. 1H NMR (400 MHz, MeOD) δ 8.82 (s, 1H), 8.71 (s, 1H), 7.97 (s, 1H), 7.88 (d, J=8.5 Hz, 2H), 7.67 (d, J=8.4 Hz, 2H), 7.62 (d, J=8.6 Hz, 2H), 7.41 (d, J=8.5 Hz, 2H), 3.56 (s, 4H), 2.92 (dd, J=3.4 Hz, 16.8 Hz, 2H), 2.70 (dd, J=9.3 Hz, 16.8 Hz, 2H), 2.06 (td, J=6.8 Hz, 13.4 Hz, 3H), 1.10 (t, J=6.6 Hz, 6H). ESI-MS m/z 482 [M+1]. RT: 0.90 min.

### Synthesis of Compound 257

### N-(4-fluorophenyl)-N-methyl-3-[4-(1H-pyrazol-5-yl)phenyl]imidazo[1,2-a]pyrazine-6-carboxamide

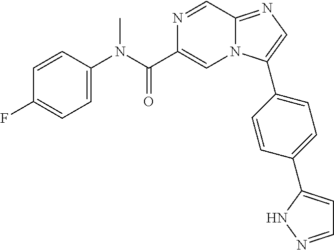 [SMILES: CN(C(=O)c1cn2c(-c3ccc(-c4ccn[nH]4)cc3)cnc2cn1)c1ccc(F)cc1]

The compound was synthesized using I-64 and an appropriate boronic acid/ester using a procedure similar Suzuki coupling protocol described in 228. MS m/z 413.1 (M+H)+; r.t.=1.428.

### Synthesis of Compound 258

### N-(3,4-difluorophenyl)-N-methyl-3-[4-(1H-pyrazol-5-yl)phenyl]imidazo[1,2-a]pyrazine-6-carboxamide

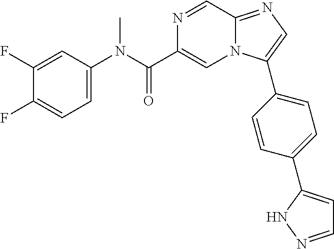 [SMILES: CN(C(=O)c1cn2c(-c3ccc(-c4ccn[nH]4)cc3)cnc2cn1)c1ccc(F)c(F)c1]

The compound was synthesized using I-65 and an appropriate boronic acid/ester using a procedure similar Suzuki coupling protocol described in 228. MS m/z 431.1 (M+H)+; r.t.=1.477.

### Synthesis of Compound 259

### 4-chloro-N-methyl-N-{3-[4-(trifluoromethyl)phenyl]imidazo[1,2-a]pyrazin-6-yl}benzamide

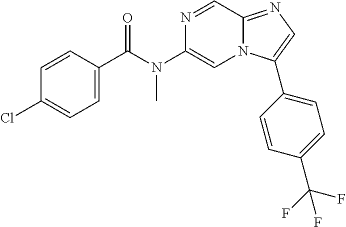 [SMILES: CN(C(=O)c1ccc(Cl)cc1)c1cn2c(-c3ccc(C(F)(F)F)cc3)cnc2cn1]

The compound was synthesized using a procedure analogous to one use for synthesis of 24. 1H NMR (400 MHz, CDCl₃) δ 9.13 (d, J=1.2, 1H), 7.92 (s, 1H), 7.88 (s, 1H), 7.72 (d, J=8.2, 2H), 7.35 (q, J=8.7, 4H), 7.23 (d, J=8.0, 2H), 3.56 (s, 3H). HRMS calcd for C₂₁H₁₅ClF₃N₄O [M+H]+431.08; found 431.1.

### Synthesis of Compound 260

### methyl N-(5-{6-[(4-cyanophenyl)(methyl)carbamoyl]imidazo[1,2-a]pyrazin-3-yl}pyridin-2-yl)carbamate

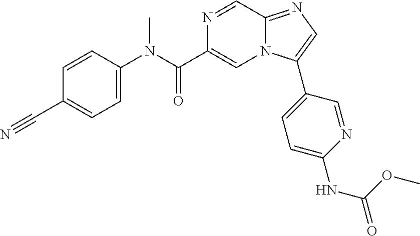 [SMILES: COC(=O)Nc1ccc(-c2cnc3cnc(C(=O)N(C)c4ccc(C#N)cc4)cn23)cn1]

The compound was synthesized using I-59 and an appropriate boronic acid/ester using a procedure similar Suzuki coupling protocol described in 228. M/Z=428.1 (M+1), r.t=1.22 mins.

### Synthesis of Compound 261

### Propan-2-yl N-(4-{6-[(4-cyanophenyl)(methyl)carbamoyl]imidazo[1,2-a]pyrazin-3-yl}phenyl)carbamate

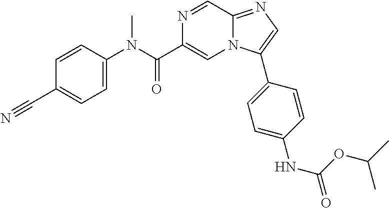 [SMILES: CC(C)OC(=O)Nc1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. M/Z=455.20 (M+1), r.t=1.65 mins.

### Synthesis of Compound 262

### N-(6-chloropyridin-3-yl)-N-methyl-3-{4-[5-(methylamino)-1,3,4-thiadiazol-2-yl]phenyl}imidazo[1,2-a]pyrazine-6-carboxamide

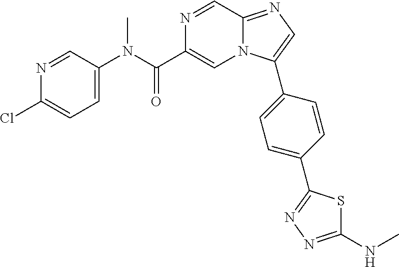 [SMILES: CNc1nnc(-c2ccc(-c3cnc4cnc(C(=O)N(C)c5ccc(Cl)nc5)cn34)cc2)s1]

The compound was synthesized using I-57 and I-60 using a procedure similar Suzuki coupling protocol described in 228. M/Z=477.2 (M+1), r.t=1.26 mins.

### Synthesis of Compound 263

### N,4-dimethyl-N-({3-[4-(trifluoromethyl)phenyl]imidazo[1,2-a]pyrazin-6-yl}methyl)aniline

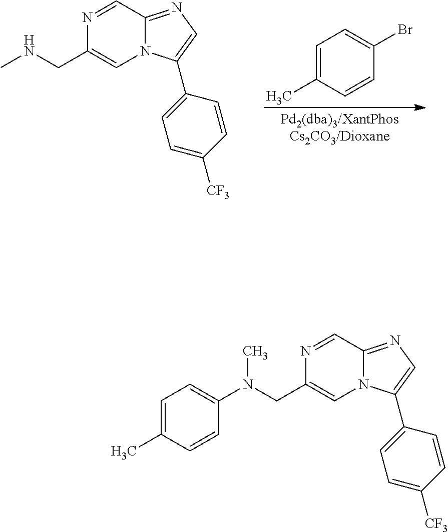

A vial containing the amine (17 mg, 0.057 mmol), aryl bromide (10 mg, 0.057 mmol), cesium carbonate (27 mg, 0.11 mmol), and dioxane (1.0 mL) was degassed under vacuum. Pd₂(dba)₃ (3 mg, 0.003 mmol) and XantPhos (4 mg, 0.006 mmol) were added. The vial was evacuated until gas evolution ceased. The evacuated vial was heated to 100° C. for 4 hours. After cooling to room temperature, the reaction was diluted with dichloromethane (1 mL). The mixture was filtered, and the filtrate concentrated under reduced pressure. The crude material was purified by preparatory TLC. LCMS analysis of the major band showed the same product mixture. The material was purified a second time using mass trigger HPLC (10 minute method, 20-70% water/acetonitrile). MS m/z 397.2 (M+H)+; r.t.=1.771.

### Synthesis of Compound 264

### 5-chloro-N-methyl-N-{3-[4-(trifluoromethyl)phenyl]imidazo[1,2-a]pyrazin-6-yl}pyridine-2-carboxamide

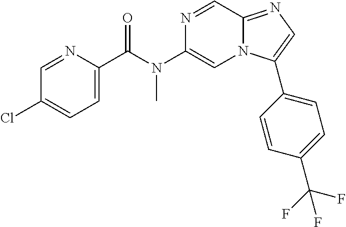 [SMILES: CN(C(=O)c1ccc(Cl)cn1)c1cn2c(-c3ccc(C(F)(F)F)cc3)cnc2cn1]

The compound was synthesized using a procedure analogous to one use for synthesis of 24. M/Z=432.1 (M+1), r.t=1.78 mins.

### Synthesis of Compound 265

### N-(6-chloropyridin-3-yl)-3-[4-(difluoromethyl)phenyl]-N-methylimidazo[1,2-a]pyrazine-6-carboxamide

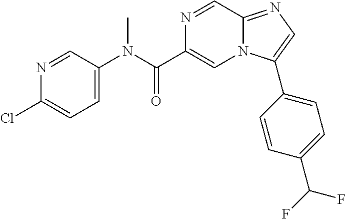 [SMILES: CN(C(=O)c1cn2c(-c3ccc(C(F)F)cc3)cnc2cn1)c1ccc(Cl)nc1]

The compound was synthesized using I-57 and an appropriate boronic acid/ester using a procedure similar Suzuki coupling protocol described in 228. M/Z=414.10 (M+1), r.t=1.51 mins.

### Synthesis of Compound 266

### 3-{4-[(2S)-2-amino-3-methylbutanamido]phenyl}-N-(4-cyanophenyl)-N-methylimidazo[1,2-a]pyrazine-6-carboxamide

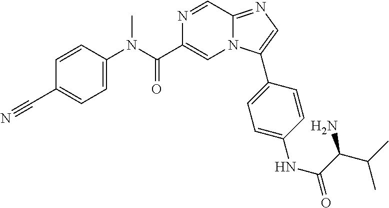 [SMILES: CC(C)[C@H](N)C(=O)Nc1ccc(-c2cnc3cnc(C(=O)N(C)c4ccc(C#N)cc4)cn23)cc1]

The compound was synthesized using a protocol described in 212. 1H NMR (400 MHz, MeOD) δ 8.85 (d, J=8.9 Hz, 1H), 8.71 (s, 1H), 7.99 (s, 1H), 7.89 (d, J=8.6 Hz, 2H), 7.73-7.61 (m, 4H), 7.42 (d, J=8.6 Hz, 2H), 3.84 (d, J=5.8 Hz, 1H), 3.56 (s, 3H), 2.45-2.30 (m, 1H), 1.16 (dd, J=6.9 Hz, 13.9 Hz, 6H), M/Z 468.1 [M+1], rt: 1.12 min.

### Synthesis of Compound 267

### 6-chloro-N-methyl-N-{3-[4-(trifluoromethyl)phenyl]imidazo[1,2-a]pyrazin-6-yl}pyridine-3-carboxamide

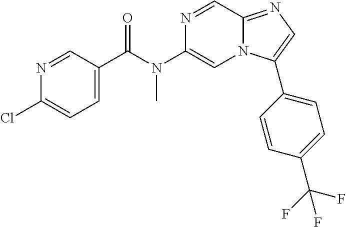 [SMILES: CN(C(=O)c1ccc(Cl)nc1)c1cn2c(-c3ccc(C(F)(F)F)cc3)cnc2cn1]

The compound was synthesized using a procedure analogous to one use for synthesis of 24. M/Z=432.1 (M+1), r.t=1.76 mins.

### Synthesis of Compound 268

### 4-fluoro-N-methyl-N-{3-[4-(trifluoromethyl)phenyl]imidazo[1,2-a]pyrazin-6-yl}benzamide

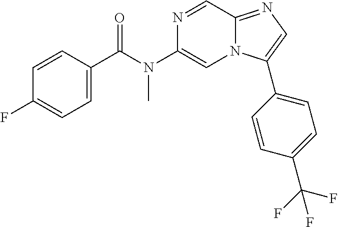 [SMILES: CN(C(=O)c1ccc(F)cc1)c1cn2c(-c3ccc(C(F)(F)F)cc3)cnc2cn1]

The compound was synthesized using a procedure analogous to one use for synthesis of 24. M/Z=415.1 (M+1), r.t=1.93 mins.

### Synthesis of Compound 269

### 3-{4-[(3R)-3-aminobutanamido]phenyl}-N-(4-cyanophenyl)-N-methylimidazo[1,2-a]pyrazine-6-carboxamide

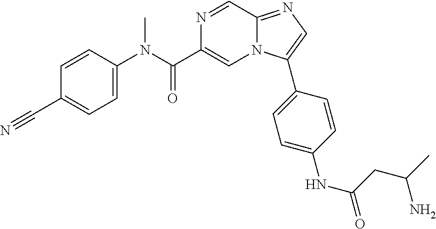 [SMILES: CC(N)CC(=O)Nc1ccc(-c2cnc3cnc(C(=O)N(C)c4ccc(C#N)cc4)cn23)cc1]

The compound was synthesized using a protocol described in 212. 1H NMR (400 MHz, MeOD) δ 8.82 (s, 1H), 8.71 (s, 1H), 7.97 (s, 1H), 7.87 (d, J=8.6 Hz, 2H), 7.65 (dd, J=8.6 Hz, 3H), 7.41 (d, J=8.5 Hz, 2H), 3.88-3.71 (m, 1H), 3.56 (s, 3H), 2.99-2.64 (m, 3H), 1.42 (d, J=6.7 Hz, 3H). M/Z 454.1 [M+1]. RT: 0.90 min.

### Synthesis of Compound 270

### 3-[4-(2-amino-2-cyclobutylacetamido)phenyl]-N-(4-cyanophenyl)-N-methylimidazo[1,2-a]pyrazine-6-carboxamide

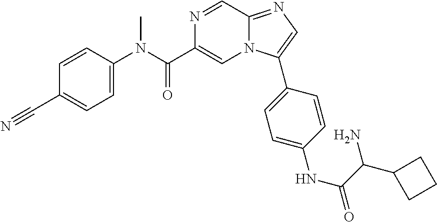 [SMILES: CN(C(=O)c1cn2c(-c3ccc(NC(=O)C(N)C4CCC4)cc3)cnc2cn1)c1ccc(C#N)cc1]

The compound was synthesized using a protocol described in 212. M/Z 480.20 [M+1]. RT: 0.93 min.

### Synthesis of Compound 271

### 3-{4-[(2S)-2-aminopropanamido]phenyl}-N-(4-cyanophenyl)-N-methylimidazo[1,2-a]pyrazine-6-carboxamide

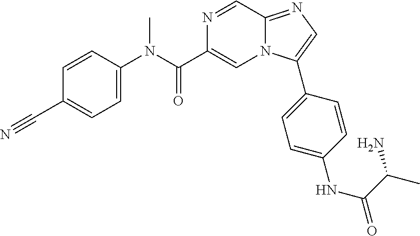 [SMILES: C[C@@H](N)C(=O)Nc1ccc(-c2cnc3cnc(C(=O)N(C)c4ccc(C#N)cc4)cn23)cc1]

The compound was synthesized using a protocol described in 212. M/Z 440.20 [M+1]. RT: 1.08 min.

### Synthesis of Compound 272

### 3-(6-aminopyridin-3-yl)-N-(4-cyanophenyl)-N-methylimidazo[1,2-a]pyrazine-6-carboxamide

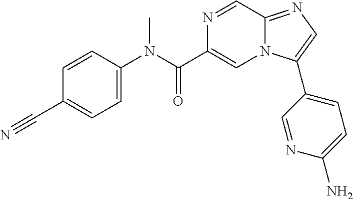 [SMILES: CN(C(=O)c1cn2c(-c3ccc(N)nc3)cnc2cn1)c1ccc(C#N)cc1]

The compound was synthesized using I-59 and an appropriate boronic acid/ester using a procedure similar Suzuki coupling protocol described in 228. M/Z=370.2 (M+1), r.t=0.91 mins.

### Synthesis of Compound 273

### 3-[4-(cyanomethoxy)phenyl]-N-(4-cyanophenyl)-N-methylimidazo[1,2-a]pyrazine-6-carboxamide

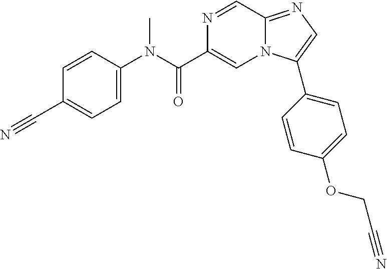 [SMILES: CN(C(=O)c1cn2c(-c3ccc(OCC#N)cc3)cnc2cn1)c1ccc(C#N)cc1]

The compound was synthesized using I-59 and an appropriate boronic acid/ester using a procedure similar Suzuki coupling protocol described in 228. M/Z=409.2 (M+1), r.t=1.52 mins.

### Synthesis of Compound 274

### 1-({3-[4-(trifluoromethyl)phenyl]imidazo[1,2-a]pyrazin-6-yl}carbonyl)-1,2,3,4-tetrahydroquinoline-6-carbonitrile

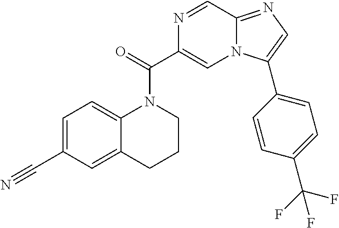 [SMILES: N#Cc1ccc2c(c1)CCCN2C(=O)c1cn2c(-c3ccc(C(F)(F)F)cc3)cnc2cn1]

The compound was synthesized using a procedure analogous to one use for synthesis of 72 (method II). MS m/z 448.1 (M+H)+; r.t.=1.699.

### Synthesis of Compound 275

### N-(6-chloropyridin-3-yl)-3-[2-fluoro-4-(trifluoromethyl)phenyl]-N-methylimidazo[1,2-a]pyrazine-6-carboxamide

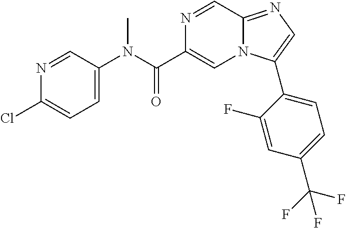 [SMILES: CN(C(=O)c1cn2c(-c3ccc(C(F)(F)F)cc3F)cnc2cn1)c1ccc(Cl)nc1]

The compound was synthesized using I-57 and an appropriate boronic acid/ester using a procedure similar Suzuki coupling protocol described in 228. M/Z=450.1 (M+1), r.t=1.64 mins.

### Synthesis of Compound 276

### N-methyl-4-(trifluoromethyl)-N-{3-[4-(trifluoromethyl)phenyl]imidazo[1,2-a]pyrazin-6-yl}benzamide

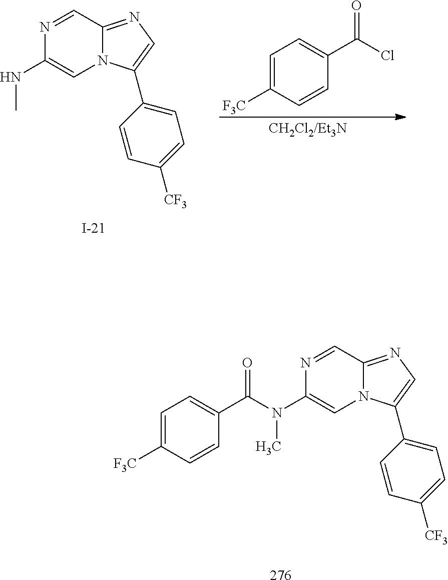

4-trifluoromethyl benzoyl chloride (1 equiv.) was added to a solution of N-methylamino imidazolopyrazine (1 equiv.), triethylamine (5 equiv.) and dichloromethane at room temperature. After 1 hour at room temperature, the solvent was removed and the crude reaction mixture was purified by mass-trigger HPLC. 1H NMR (400 MHz, CDCl₃) δ 9.17 (s, 1H), 7.93 (s, 1H), 7.72 (d, J=8.1, 2H), 7.64 (d, J=8.1, 1H), 7.55 (d, J=8.2, 2H), 7.28 (d, J=6.8, 2H), 7.22 (d, J=6.5, 2H), 3.56 (s, J=9.0, 3H). HRMS calcd for C₂₂H₁₅F₆N₄O [M+H]+465.11; found 465.2.

### Synthesis of Compound 277

### N-methyl-N-(6-methylpyridin-3-yl)-3-[4-(trifluoromethyl)phenyl]imidazo[1,2-a]pyrazine-6-carboxamide

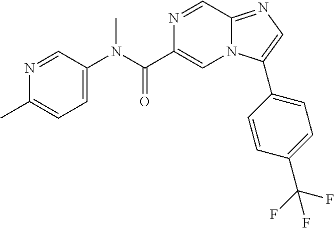 [SMILES: Cc1ccc(N(C)C(=O)c2cn3c(-c4ccc(C(F)(F)F)cc4)cnc3cn2)cn1]

The compound was synthesized using a procedure analogous to one use for synthesis of 72 (method II). ¹H NMR (400 MHz, MeOD) δ 9.08 (d, J=1.3, 1H), 8.72 (d, J=29.4, 2H), 8.16 (s, 2H), 7.96 (s, 3H), 7.69 (d, J=8.6, 2H), 3.59 (s, 3H), 2.69 (s, 3H). ESI-MS m/z 412 [M+1]. RT: 1.25 min.

### Synthesis of Compound 278

### 3-{4-[(2-amino-2-methylpropyl)amino]phenyl}-N-(4-cyanophenyl)-N-methylimidazo[1,2-a]pyrazine-6-carboxamide

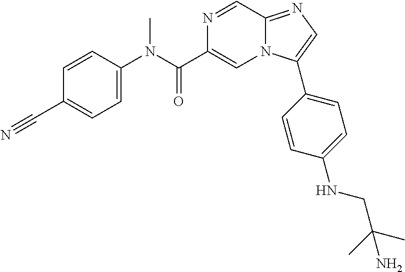 [SMILES: CN(C(=O)c1cn2c(-c3ccc(NCC(C)(C)N)cc3)cnc2cn1)c1ccc(C#N)cc1]

To a solution of 124 in EtOH (0.03 g, 0.07 mmols), was added the aldehyde (0.175 mmols, 0.035 g, 2.5 eq) followed by HOAc (2 drops), and the mixture was heated at 80° C. for 4 hrs. The reaction mixture was cooled down to 0° C., added the NaBH(OAc)₃ and stirred overnight. Next day after checking via LC/MS for the desired mass, the solvents were removed under vacuo, the compound was re-dissolved in TFA/DCM (1:1) and stirred for ½ hr. Next solvents were removed once again and the desired compound was isolated via mass triggered HPLC. M/Z=440.2 (M+1), r.t=1.14 mins.

### Synthesis of Compound 279

### N-(6-chloropyridin-3-yl)-3-(4-acetamidophenyl)-N-methylimidazo[1,2-a]pyrazine-6-carboxamide

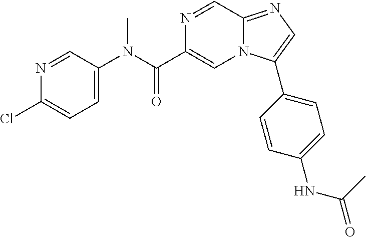 [SMILES: CC(=O)Nc1ccc(-c2cnc3cnc(C(=O)N(C)c4ccc(Cl)nc4)cn23)cc1]

The compound was synthesized using I-57 and an appropriate boronic acid/ester using a procedure similar Suzuki coupling protocol described in 228. M/Z=421.1 (M+1), r.t=1.16 mins.

### Synthesis of Compound 280

### N-(6-chloropyridin-3-yl)-3-(2-fluoro-4-methoxyphenyl)-N-methylimidazo[1,2-a]pyrazine-6-carboxamide

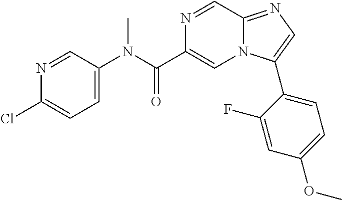 [SMILES: COc1ccc(-c2cnc3cnc(C(=O)N(C)c4ccc(Cl)nc4)cn23)c(F)c1]

The compound was synthesized using I-57 and an appropriate boronic acid/ester using a procedure similar Suzuki coupling protocol described in 228. M/Z=421.1 (M+1), r.t=1.16 mins.

### Synthesis of Compound 281

### 7-fluoro-4-({3-[4-(trifluoromethyl)phenyl]imidazo[1,2-a]pyrazin-6-yl}methyl)-3,4-dihydro-2H-1,4-benzoxazine

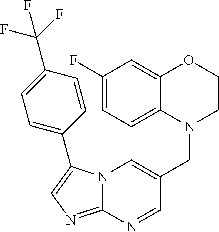 [SMILES: Fc1ccc2c(c1)OCCN2Cc1cnc2ncc(-c3ccc(C(F)(F)F)cc3)n2c1]

The compound was synthesized using I-73 and an appropriate boronic acid/ester using a procedure similar Suzuki coupling protocol described in 228. ¹H NMR (400 MHz, CD₃OD): δ 9.09 (s, 1H), 8.42 (s, 1H), 8.04 (s, 1H), 7.77-7.84 (m, 4H), 6.75-6.79 (m, 1H), 6.49-6.53 (m, 2H), 4.6 (s, 2H), 4.24 (t, J=4.39 Hz, 2H), 3.45 (t, J=4.39 Hz, 2H). LC-MS: 98.06%; 429.14 (M+H).

### Synthesis of Compound 282

### 3-{4-[(2R)-2-aminopropanamido]phenyl}-N-(4-cyanophenyl)-N-methylimidazo[1,2-a]pyrazine-6-carboxamide

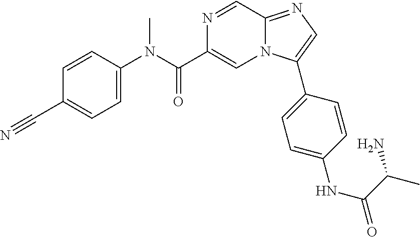 [SMILES: C[C@@H](N)C(=O)Nc1ccc(-c2cnc3cnc(C(=O)N(C)c4ccc(C#N)cc4)cn23)cc1]

The compound was synthesized using a protocol described in 212. M/Z=440.1 (M+1), r.t=1.01 mins.

### Synthesis of Compound 283

### 4-cyano-N-methyl-N-{3-[4-(trifluoromethyl)phenyl]imidazo[1,2-a]pyrazin-6-yl}benzamide

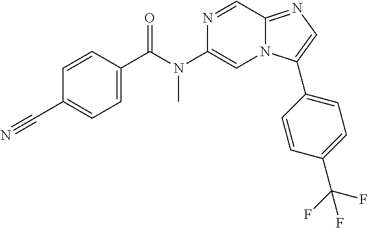 [SMILES: CN(C(=O)c1ccc(C#N)cc1)c1cn2c(-c3ccc(C(F)(F)F)cc3)cnc2cn1]

The compound was synthesized using a procedure analogous to one use for synthesis of 24 M/Z=421.1 (M+1), r.t=1.76 mins.

### Synthesis of Compound 284

### N-(4-cyanophenyl)-3-{4-[2-(dimethylamino)ethoxy]phenyl}-N-methylimidazo[1,2-a]pyrazine-6-carboxamide

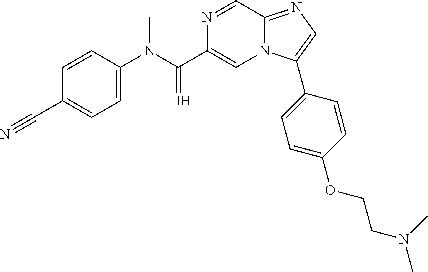

The compound was synthesized using I-59 and an appropriate boronic acid/ester using a procedure similar Suzuki coupling protocol described in 228. ¹H NMR (400 MHz, CDCl3) δ 8.83 (s, 1H), 8.76 (s, 1H), 7.90 (s, 1H), 7.62 (d, J=8.6 Hz, 2H), 7.51 (d, J=8.7 Hz, 2H), 7.25 (d, J=8.5 Hz, 2H), 7.12 (d, J=8.7 Hz, 2H), 4.57-4.48 (m, 2H), 3.65-3.53 (m, 5H), 3.02 (s, 6H). ESI-MS m/z 441 [M+1]. RT: 1.11 min.

### Synthesis of Compound 285

### N-(4-cyanophenyl)-N,2-dimethyl-3-[4-(trifluoromethyl)phenyl]imidazo[1,2-a]pyrazine-6-carboxamide

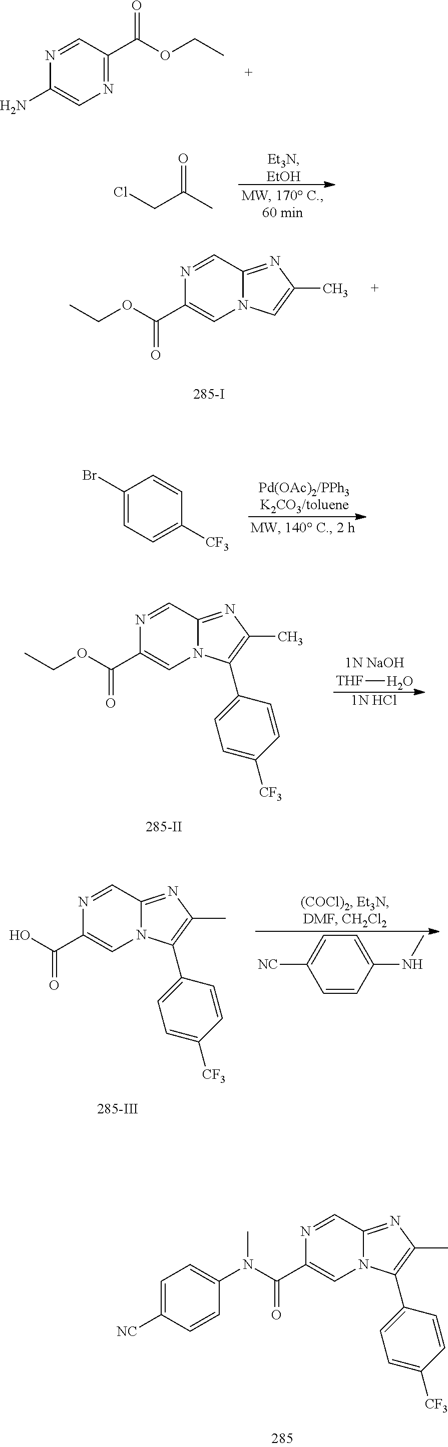

### Ethyl 2-methylimidazo[1,2-a]pyrazine-6-carboxylate 285-I

To a solution of ethyl 5-aminopyrazine-2-carboxylate (668 mg, 4 mmol) in EtOH (25 mL) was added 1-chloropropan-2-one (0.32 mL, 4 mmol) and triethylamine (0.83 mL, 6 mmol) and the mixture was heated in microwave synthesizer at 170° C. for 30 min. The reaction mixture was cooled to RT and solvent was removed. The residue was purified by flash chromatography (silica gel, EtOAc/hexanes) to afford 230 mg (28%) of I as white solid. ¹H NMR (400 MHz, CDCl3) δ 9.03 (s, 1H), 8.92 (d, J=1.3, 1H), 7.59 (s, 1H), 4.51 (q, J=7.1, 2H), 2.61-2.53 (m, 3H), 1.47 (t, J=7.1, 3H). HRMS calcd for C₁₀H₁₂N₃O₂ [M+H]⁺ 205.09; found 206.1.

### Ethyl 2-methyl-3-(4-(trifluoromethyl)phenyl)imidazo[1,2-a]pyrazine-6-carboxylate 285-II

To a solution of Ethyl 2-methylimidazo[1,2-a]pyrazine-6-carboxylate I (102 mg, 0.5 mmol) in toluene was added 1-bromo-4-(trifluoromethyl)benzene (135 mg, 0.6 mmol), Pd(OAc)₂ (12 mg, 0.3 mmol), PPh₃ (26 mg, 0.1 mmol) and K₂CO₃ (136 mg, 1.0 mmol). The reaction j mixture was purged with N₂ for 2 min and heated in microwave synthesizer at 140° C. for 2 h. After cooling to rt, the reaction mixture was filtered through celite and washed with ethylacetate and solvent was removed. The dark brown residue was purified by flash chromatography (silica gel, EtOAc/hexanes) to afford 104 mg (59%) of 285-II as off white solid. ¹H NMR (400 MHz, MeOD) δ 9.02 (s, 1H), 8.92 (s, 1H), 7.98 (d, J=8.2, 2H), 7.86 (d, J=8.1, 2H), 4.10 (q, J=7.1, 2H), 4.10 (q, J=7.1, 1H), 2.60 (s, 3H), 1.25 (t, J=7.1, 3H). HRMS calcd for C₁₇H₁₅F₃N₃O₂ [M+H]⁺ 349.10; found 350.10.

### 2-Methyl-3-(4-(trifluoromethyl)phenyl)imidazo[1,2-a]pyrazine-6-carboxylic acid 285-III

To a solution of ethyl 2-methyl-3-(4-(trifluoromethyl)phenyl)imidazo[1,2-a]pyrazine-6-carboxylate 285-II (102 mg, 0.2 mmol) in MeOH-THF (2:1 mixture, 2 mL) was added 1N NaOH (0.5 mL, 0.5 mmol) and the reaction mixture was stirred at rt for 3 h. THF was removed and the reaction mixture was acidified with 1N HCl. The resulting precipitate was collected by filtration and dried to afford 58 mg (82%) of III as white solid. HRMS calcd for C₂₅H₁₁F₃N₃O₂ [M+H] 321.07; found 322.10.

### N-(4-cyanophenyl)-N,2-dimethyl-3-(4-(trifluoromethyl)phenyl)imidazole[1,2-a]pyrazine-6-carboxamide (285)

To a solution of 2-Methyl-3-(4-(trifluoromethyl)phenyl)imidazole[1,2-a]pyrazine-6-carboxylic acid 285-III (25 mg, 0.08 mmol) in CH₂Cl₂ (2 mL) was added a drop of DMF and the reaction mixture was cooled to 0° C. Oxallylchloride (10 μL, 0.1 mmol) was added and stirred it for 30 min. Solvents removed in vacuo and the residue was again dissolved in CH₂Cl₂ (2 mL) and 4-(methylamino)benzonitrile (12 mg, 0.1 mmol) was added and the reaction mixture left it at RT overnight. Solvent was removed and purified by Prep LCMS (TFA-H₂O, CH₃CN). ¹H NMR (400 MHz, MeOD) δ 8.69 (s, 1H), 8.65 (s, 1H), 7.96 (d, J=8.2, 2H), 7.78 (d, J=8.1, 2H), 7.72 (d, J=8.4, 2H), 7.40 (d, J=8.6, 2H), 3.54 (s, 3H), 2.54 (s, 3H). HRMS calcd for C₂₃H₁₇F₃N₅O [M+H] 435.14; found 435.10.

### Synthesis of Compound 286

### 3-{4-[(2R)-2-amino-3-methylbutanamido]phenyl}-N-(4-cyanophenyl)-N-methylimidazo[1,2-a]pyrazine-6-carboxamide

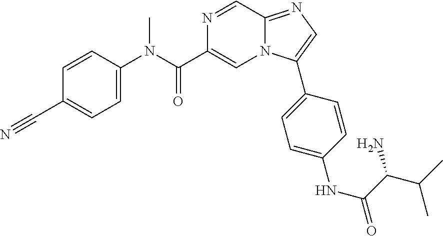 [SMILES: CC(C)[C@@H](N)C(=O)Nc1ccc(-c2cnc3cnc(C(=O)N(C)c4ccc(C#N)cc4)cn23)cc1]

The compound was synthesized using a protocol described in 212. ¹H NMR (400 MHz, CDCl3) δ 8.83 (s, 1H), 8.76 (s, 1H), 7.90 (s, 1H), 7.62 (d, J=8.6 Hz, 2H), 7.51 (d, J=8.7 Hz, 2H), 7.25 (d, J=8.5 Hz, 2H), 7.12 (d, J=8.7 Hz, 2H), 4.57-4.48 (m, 2H), 3.65-3.53 (m, 5H), 3.02 (s, 6H). ESI-MS m/z 441 [M+1]. RT: 1.11 min.

### Synthesis of Compound 287

### N,5-dimethyl-N-({3-[4-(trifluoromethyl)phenyl]imidazo[1,2-a]pyrazin-6-yl}methyl)pyridin-2-amine

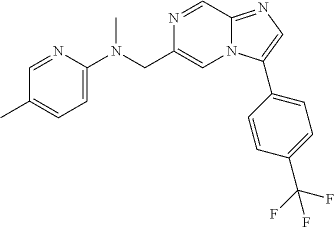 [SMILES: Cc1ccc(N(C)Cc2cn3c(-c4ccc(C(F)(F)F)cc4)cnc3cn2)nc1]

The compound was synthesized using a procedure analogous to one use for synthesis of I-15. MS m/z 398.2 (M+H)+; r.t.=1.395.

### Synthesis of Compound 288

### N-(6-methoxy-5-methylpyridin-3-yl)-N-methyl-3-[4-(trifluoromethyl)phenyl]imidazo[1,2-a]pyrazine-6-carboxamide

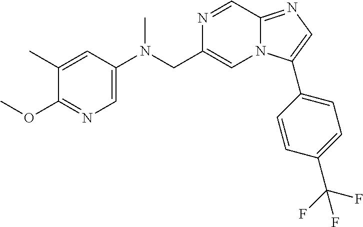 [SMILES: COc1ncc(N(C)Cc2cn3c(-c4ccc(C(F)(F)F)cc4)cnc3cn2)cc1C]

The compound was synthesized using a procedure analogous to one use for synthesis of 72 (method II). M/Z=442.2 (M+1), r.t=1.83 mins.

### Synthesis of Compound 289

### N,4-dimethyl-N-{3-[4-(trifluoromethyl)phenyl]imidazo[1,2-a]pyrazin-6-yl}benzamide

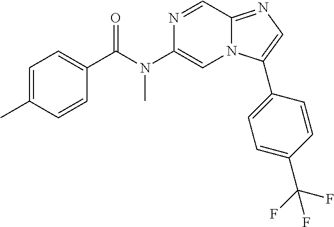 [SMILES: Cc1ccc(C(=O)N(C)c2cn3c(-c4ccc(C(F)(F)F)cc4)cnc3cn2)cc1]

The compound was synthesized using a procedure analogous to one use for synthesis of 24. 1H NMR (400 MHz, CDCl3) δ 9.05 (d, J=1.2, 1H), 7.82 (s, 1H), 7.70 (s, 1H), 7.59 (d, J=8.2, 2H), 7.25 (d, J=8.1, 2H), 7.10 (d, J=8.0, 2H), 7.03 (d, J=8.0, 2H), 3.50 (s, 3H), 2.30 (d, J=29.4, 3H). HRMS calcd for C₂₂H₁₈F₃N₄O [M+H]+411.14; found 411.1.

### Synthesis of Compound 290

### N-(6-methoxypyridin-2-yl)-N-methyl-3-[4-(trifluoromethyl)phenyl]imidazo[1,2-a]pyrazine-6-carboxamide

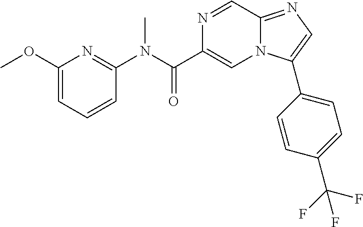 [SMILES: COc1cccc(N(C)C(=O)c2cn3c(-c4ccc(C(F)(F)F)cc4)cnc3cn2)n1]

The compound was synthesized using a procedure analogous to one use for synthesis of 72 (method II). M/Z=428.1 (M+1), rt=1.87 mins.

### Synthesis of Compound 291

### N-(4-cyanophenyl)-3-(4-{[2-(dimethylamino)ethyl]carbamoyl}phenyl)-N-methylimidazo[1,2-a]pyrazine-6-carboxamide

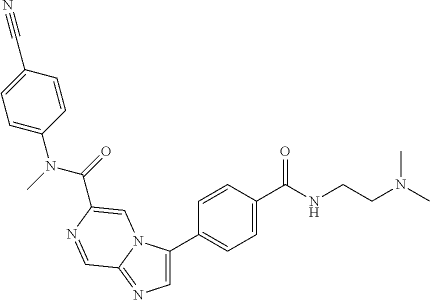 [SMILES: CN(C)CCNC(=O)c1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. M/Z=468.2 (M+1), r.t=0.88 mins.

### Synthesis of Compound 292

### 3-{4-[(2S)-2-amino-2-cyclopropylacetamido]phenyl}-N-(4-cyanophenyl)-N-methylimidazo[1,2-a]pyrazine-6-carboxamide

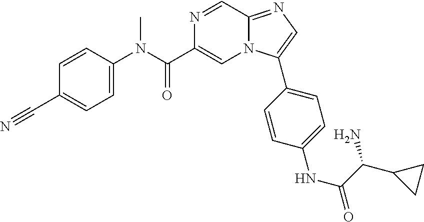 [SMILES: CN(C(=O)c1cn2c(-c3ccc(NC(=O)[C@H](N)C4CC4)cc3)cnc2cn1)c1ccc(C#N)cc1]

The compound was synthesized using a protocol described in 212. ¹H NMR (400 MHz, MeOD) δ 8.73 (s, 1H), 8.61 (s, 1H), 7.88 (s, 1H), 7.80 (s, 2H), 7.54 (s, 4H), 7.32 (s, 2H), 3.45 (s, 3H), 3.31 (s, 1H), 1.22 (s, 2H), 0.72 (s, 3H), 0.50 (s, 1H). ESI-MS m/z 466 [M+1]+. RT: 1.08 min.

### Synthesis of Compound 293

### N-(4-cyano-2-fluorophenyl)-N-methyl-3-[4-(trifluoromethyl)phenyl]imidazo[1,2-a]pyrazine-6-carboxamide

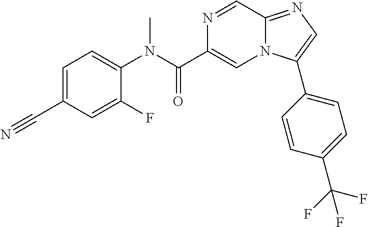 [SMILES: CN(C(=O)c1cn2c(-c3ccc(C(F)(F)F)cc3)cnc2cn1)c1ccc(C#N)cc1F]

The compound was synthesized using a procedure analogous to one use for synthesis of 72 (method II). MS m/z 440.1 (M+H)+; r.t.=1.757.

### Synthesis of Compound 294

### N,4,4-trimethyl-N-{3-[4-(trifluoromethyl)phenyl]imidazo[1,2-a]pyrazin-6-yl}piperidine-1-carboxamide

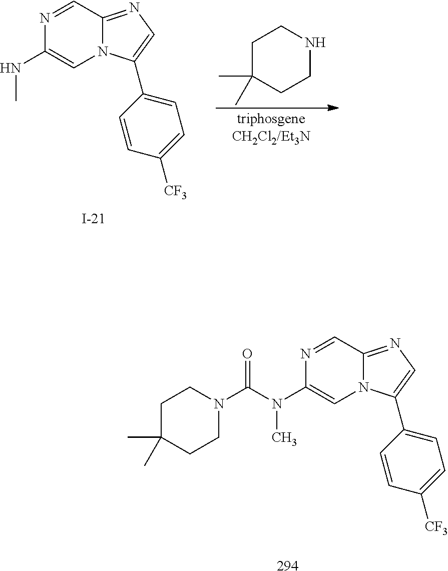

A solution of NHMe core I-21 (1.0 equiv.) in dichloromethane was treated with triphosgene (0.5 equiv.) and allowed to stir at rt for 3 h. The amine (1.5 equiv.) was added, followed by Et3N (5.0 equiv.), and the resulting mixture was allowed to stir at rt overnight. The reaction mixture was concentrated, redissolved in 10% MeOH/DMSO, and purified by mass-triggered HPLC. ¹H NMR (400 MHz, CDCl3) δ 9.12 (d, J=1.3, 1H), 8.23 (d, J=1.3, 1H), 7.92 (s, 1H), 7.82 (d, J=8.1, 2H), 7.68 (d, J=8.1, 2H), 3.31 (s, 3H), 3.30-3.24 (m, 4H), 1.24-1.20 (m, 4H), 0.87 (s, 6H). MS m/z 432.2; (M+H)+; r.t.=1.91.

### Synthesis of Compound 295

### 3-[6-(2-aminoacetamido)pyridin-3-yl]-N-(4-cyanophenyl)-N-methylimidazo[1,2-a]pyrazine-6-carboxamide

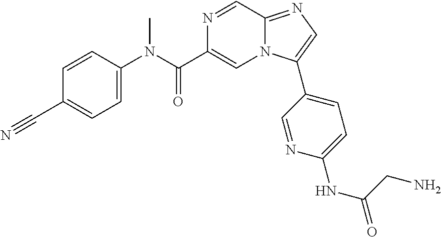 [SMILES: CN(C(=O)c1cn2c(-c3ccc(NC(=O)CN)nc3)cnc2cn1)c1ccc(C#N)cc1]

The compound was synthesized using a procedure analogous to one use for synthesis of 212. ¹H NMR (400 MHz, MeOD) δ 8.89 (d, J=1.3 Hz, 1H), 8.80 (s, 1H), 8.25 (s, 1H), 8.21-8.16 (m, 1H), 8.14 (s, 1H), 7.67 (d, J=8.6 Hz, 2H), 7.41 (d, J=8.6 Hz, 2H), 7.24 (d, J=9.3 Hz, 1H), 3.56 (s, 3H), 3.31 (dt, J=1.6 Hz, 3.2 Hz, 2H). ESI-MS m/z 427 [M+1]. RT: 0.87 min.

### Synthesis of Compound 296

### N-(2-methoxypyridin-4-yl)-N-methyl-3-[4-(trifluoromethyl)phenyl]imidazo[1,2-a]pyrazine-6-carboxamide

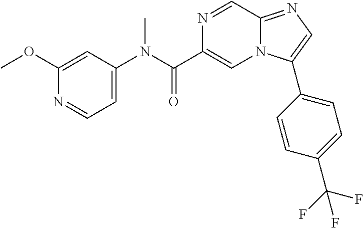 [SMILES: COc1cc(N(C)C(=O)c2cn3c(-c4ccc(C(F)(F)F)cc4)cnc3cn2)ccn1]

The compound was synthesized using a procedure analogous to one use for synthesis of 72 (method II). MS m/z 428.1 (M+H)+; r.t.=1.428.

### Synthesis of Compound 297

### N-(4-cyanophenyl)-N-methyl-3-(6-methylpyridin-3-yl)imidazo[1,2-a]pyrazine-6-carboxamide

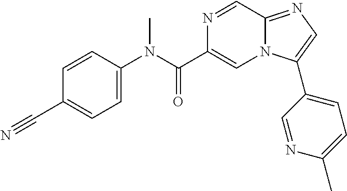 [SMILES: Cc1ccc(-c2cnc3cnc(C(=O)N(C)c4ccc(C#N)cc4)cn23)cn1]

The compound was synthesized using I-59 and an appropriate boronic acid/ester using a procedure similar Suzuki coupling protocol described in 228. M/Z=369.2 (M+1), r.t=1.34 mins.

### Synthesis of Compound 298

### N-(1-ethyl-1H-pyrazol-5-yl)-N-methyl-3-[4-(trifluoromethyl)phenyl]imidazo[1,2-a]pyrazine-6-carboxamide

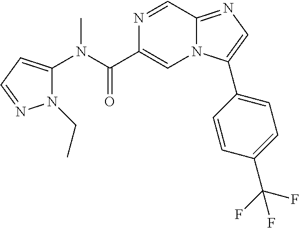 [SMILES: CCn1nccc1N(C)C(=O)c1cn2c(-c3ccc(C(F)(F)F)cc3)cnc2cn1]

The compound was synthesized using a procedure analogous to one use for synthesis of 72 (method II). M/Z=415.2 (M+1), r.t=1.61 mins.

### Synthesis of Compound 299

### N-methyl-N-(2-methylpyridin-4-yl)-3-[4-(trifluoromethyl)phenyl]imidazo[1,2-a]pyrazine-6-carboxamide

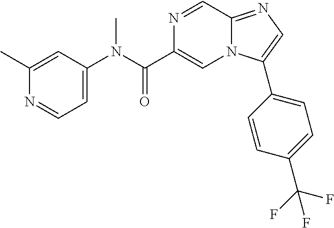 [SMILES: Cc1cc(N(C)C(=O)c2cn3c(-c4ccc(C(F)(F)F)cc4)cnc3cn2)ccn1]

The compound was synthesized using a procedure analogous to one use for synthesis of 72 (method II). MS m/z 412.2 (M+H)+; r.t.=1.164.

### Synthesis of Compound 300

### tert-butyl N-{2-[(4-{6-[(4-cyanophenyl)(methyl)carbamoyl]imidazo[1,2-a]pyrazin-3-yl}phenyl)amino]-2-methylpropyl}carbamate

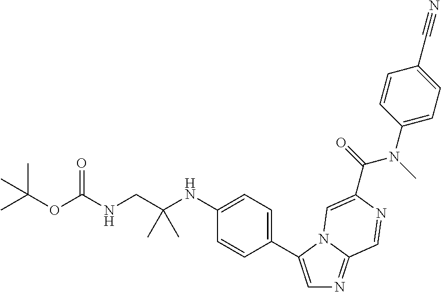 [SMILES: CN(C(=O)c1cn2c(-c3ccc(NC(C)(C)CNC(=O)OC(C)(C)C)cc3)cnc2cn1)c1ccc(C#N)cc1]

The compound was synthesized using I-59 and an appropriate boronic acid/ester using a procedure similar Suzuki coupling protocol described in 228. M/Z=540.3 (M+1), r.t=1.75 mins.

### Synthesis of Compound 301

### N-(6-chloropyridin-3-yl)-3-(1H-indol-5-yl)-N-methylimidazo[1,2-a]pyrazine-6-carboxamide

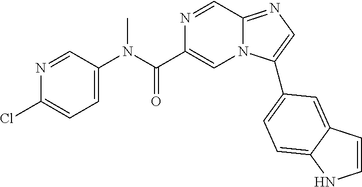 [SMILES: CN(C(=O)c1cn2c(-c3ccc4[nH]ccc4c3)cnc2cn1)c1ccc(Cl)nc1]

The compound was synthesized using I-57 and an appropriate boronic acid/ester using a procedure similar Suzuki coupling protocol described in 228. M/Z=403.1 (M+1), r.t=1.34 mins.

### Synthesis of Compound 302

### 3-(4-methoxy-2-methylphenyl)-N-[6-(4-methoxy-2-methylphenyl)pyridin-3-yl]-N-methylimidazo[1,2-a]pyrazine-6-carboxamide

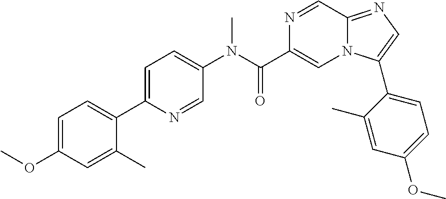

The compound was synthesized using I-19 and an appropriate boronic acid/ester using a procedure similar Suzuki coupling protocol described in 228. M/Z=494.2 (M+1), r.t=1.50 mins.

### Synthesis of Compound 303

### N-(4-methoxypyridin-3-yl)-N-methyl-3-[4-(trifluoromethyl)phenyl]imidazo[1,2-a]pyrazine-6-carboxamide

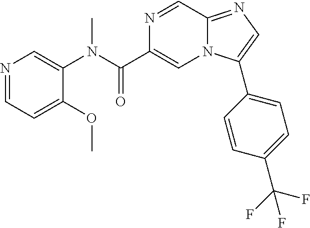 [SMILES: COc1ccncc1N(C)C(=O)c1cn2c(-c3ccc(C(F)(F)F)cc3)cnc2cn1]

The compound was synthesized using a procedure analogous to one use for synthesis of 72 (method II). M/Z=428.1 (M+1), r.t=1.33 mins.

### Synthesis of Compound 304

### N-(6-chloro-5-methylpyridin-3-yl)-N-methyl-3-[4-(trifluoromethyl)phenyl]imidazo[1,2-a]pyrazine-6-carboxamide

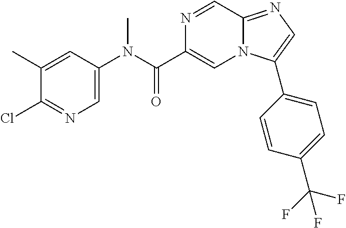 [SMILES: Cc1cc(N(C)C(=O)c2cn3c(-c4ccc(C(F)(F)F)cc4)cnc3cn2)cnc1Cl]

The compound was synthesized using a procedure analogous to one use for synthesis of 72 (method II) using I-48. MS m/z 446.1 (M+H)+; r.t.=1.726.

### Synthesis of Compound 305

### 7-fluoro-4-({3-[4-(trifluoromethyl)phenyl]imidazo[1,2-a]pyrazin-6-yl}carbonyl)-3,4-dihydro-2H-1,4-benzoxazine

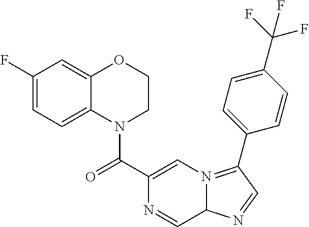

The compound was synthesized using I-74 and an appropriate boronic acid/ester using a procedure similar Suzuki coupling protocol described in 228. ¹H-NMR (400 MHz, DMSO-d₆): 9.17 (s, 1H), 8.98 (s, 1H), 8.31 (s, 1H), 8.02 (d, J=8.35 Hz, 2H), 7.97 (d, J=8.35 Hz, 2H), 7.8 (bs, 1H), 6.69-6.85 (m, 2H), 4.32 (bs, 2H), 3.96 (bs, 2H). LC-MS: 99.39%; 443.08 (M+H).

### Synthesis of Compound 306

### ({3-[4-(trifluoromethyl)phenyl]imidazo[1,2-a]pyrazin-6-yl}carbonyl)-3,4-dihydro-2H-1,4-benzoxazine

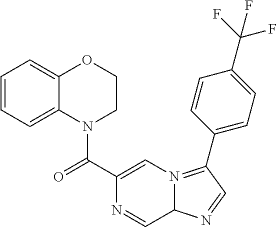

The compound was synthesized using a procedure analogous to one use for synthesis of 72 (method II). MS m/z 425.1 (M+H)+; rt.=1.781.

### Synthesis of Compound 307

### 3-(4-{[1-(aminomethyl)cyclopropane]amido}phenyl)-N-(4-cyanophenyl)-N-methylimidazo[1,2-a]pyrazine-6-carboxamide

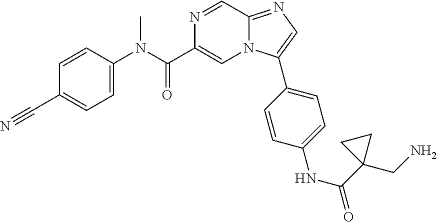 [SMILES: CN(C(=O)c1cn2c(-c3ccc(NC(=O)C4(CN)CC4)cc3)cnc2cn1)c1ccc(C#N)cc1]

The compound was synthesized using a protocol described in 212. ¹H NMR (400 MHz, MeOD) δ 8.82 (s, 1H), 8.71 (s, 1H), 7.96 (s, 1H), 7.84 (d, J=8.7 Hz, 2H), 7.68 (d, J=8.6 Hz, 2H), 7.60 (d, J=8.6 Hz, 2H), 7.41 (d, J=8.6 Hz, 2H), 3.56 (s, 3H), 3.21 (s, 2H), 1.60 (s, 2H), 1.23 (s, 2H). ESI-MS m/z 466 [M+1]. RT: 0.98 min.

### Synthesis of Compound 308

### N-methyl-6-(trifluoromethyl)-N-{3-[4-(trifluoromethyl)phenyl]imidazo[1,2-a]pyrazin-6-yl}pyridine-3-carboxamide

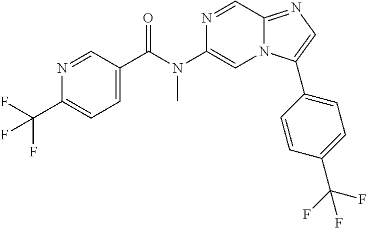 [SMILES: CN(C(=O)c1ccc(C(F)(F)F)nc1)c1cn2c(-c3ccc(C(F)(F)F)cc3)cnc2cn1]

The compound was synthesized using a procedure analogous to one use for synthesis of 24. MS m/z 466.1 (M+H)+; r.t.=1.526.

### Synthesis of Compound 309

### N-methyl-N-(4-methylpyridin-3-yl)-3-[4-(trifluoromethyl)phenyl]imidazo[1,2-a]pyrazine-6-carboxamide

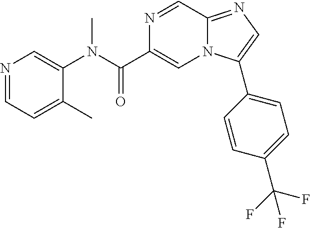 [SMILES: Cc1ccncc1N(C)C(=O)c1cn2c(-c3ccc(C(F)(F)F)cc3)cnc2cn1]

The compound was synthesized using a procedure analogous to one use for synthesis of 72 (method II). M/Z=412.2 (M+1), rt=1.33 mins.

### Synthesis of Compound 310

### N-(4-ethoxypyridin-2-yl)-N-methyl-3-[4-(trifluoromethyl)phenyl]imidazo[1,2-a]pyrazine-6-carboxamide

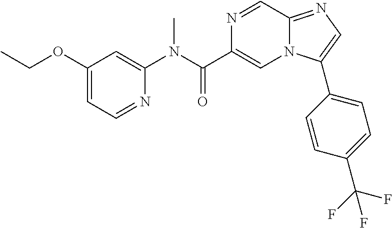 [SMILES: CCOc1ccnc(N(C)C(=O)c2cn3c(-c4ccc(C(F)(F)F)cc4)cnc3cn2)c1]

The compound was synthesized using a procedure analogous to one use for synthesis of 72 (method II). M/Z=442.2 (M+1), rt=1.60 mins.

### Synthesis of Compound 311

### 4-methoxy-N-methyl-N-{3-[4-(trifluoromethyl)phenyl]imidazo[1,2-a]pyrazin-6-yl}benzamide

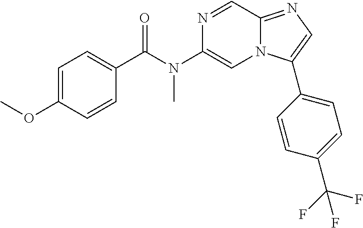 [SMILES: COc1ccc(C(=O)N(C)c2cn3c(-c4ccc(C(F)(F)F)cc4)cnc3cn2)cc1]

The compound was synthesized using a procedure analogous to one use for synthesis of 24. 1H NMR (400 MHz, CDCl₃) δ 9.09 (d, J=1.3, 1H), 7.88 (s, 1H), 7.76 (s, 1H), 7.65 (d, J=8.2, 2H), 7.37 (d, J=8.8, 2H), 7.14 (d, J=8.1, 2H), 6.83 (d, J=6.9, 2H), 3.77 (s, 3H), 3.55 (s, 3H). HRMS calcd for C₂₂H₁₈F₃N₄O₂ [M+H]+427.18; found 427.2.

### Synthesis of Compound 312

### N-(6-chloropyridin-3-yl)-N-methyl-3-{1H-pyrrolo[2,3-b]pyridin-5-yl}imidazo[1,2-a]pyrazine-6-carboxamide

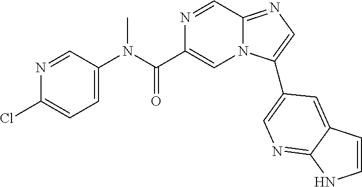 [SMILES: CN(C(=O)c1cn2c(-c3cnc4[nH]ccc4c3)cnc2cn1)c1ccc(Cl)nc1]

The compound was synthesized using I-57 and an appropriate boronic acid/ester using a procedure similar Suzuki coupling protocol described in 228. ¹H NMR (400 MHz, MeOD) δ 8.86 (s, 1H), 8.68-8.61 (m, 1H), 8.51-8.44 (m, 1H), 8.30-8.23 (m, 2H), 8.07-8.04 (m, 1H), 7.95 (s, 1H), 7.84-7.74 (m, 2H), 7.60-7.55 (m, 1H), 3.55 (s, 3H), ESI-MS m/z 404 [M+1]. RT: 1.14 min

### Synthesis of Compound 313

### N-methyl-N-[4-(1H-1,2,4-triazol-1-yl)phenyl]-3-[4-(trifluoromethyl)phenyl]imidazo[1,2-a]pyrazine-6-carboxamide

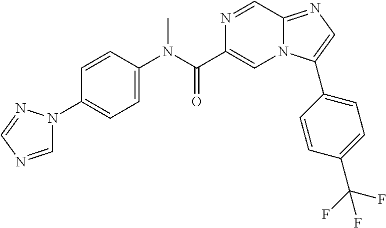 [SMILES: CN(C(=O)c1cn2c(-c3ccc(C(F)(F)F)cc3)cnc2cn1)c1ccc(-n2cncn2)cc1]

The compound was synthesized using a procedure analogous to one use for synthesis of 72 (method II). ¹H NMR (300 MHz, CDCl₃) 8.86 (d, J=1.17 Hz, 1H), 8.75 (s, 1H), 8.54 (s, 1H), 8.11 (s, 1H), 7.97 (s, 1H), 7.80-7.89 (m, J=8.21 Hz, 2H), 7.68-7.74 (m, J=8.20 Hz, 2H), 7.59-7.68 (m, 1H), 7.30 (d, J=8.50 Hz, 2H), 3.58 (s, 3H). ESI-MS m/z 464 [M+H]⁺.

### Synthesis of Compound 314

### N-(4-cyanophenyl)-3-(4-methanesulfonamidophenyl)-N-methylimidazo[1,2-a]pyrazine-6-carboxamide

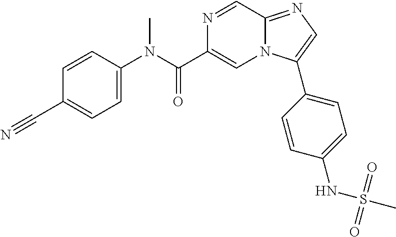 [SMILES: CN(C(=O)c1cn2c(-c3ccc(NS(C)(=O)=O)cc3)cnc2cn1)c1ccc(C#N)cc1]

The compound was synthesized using I-59 and an appropriate boronic acid/ester using a procedure similar Suzuki coupling protocol described in 228. M/Z=447.1 (M+1), r.t=1.26 mins.

### Synthesis of Compound 315

### 4-({3-[4-(trifluoromethyl)phenyl]imidazo[1,2-a]pyrazin-6-yl}carbonyl)-3,4-dihydro-2H-1,4-benzoxazine-7-carbonitrile

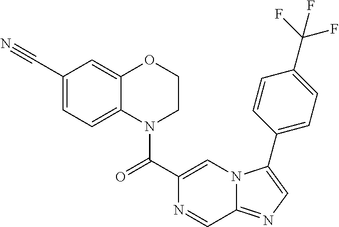 [SMILES: N#Cc1ccc2c(c1)OCCN2C(=O)c1cn2c(-c3ccc(C(F)(F)F)cc3)cnc2cn1]

Oxalyl chloride (0.10 mL of a 2.0 M solution in dichloromethane) was added dropwise to a solution of carboxylic acid (20 mg, 0.065 mmol), DMF (1 drop) and dichloromethane (1.0 mL). After 5 minutes at room temperature, the solvent was removed under reduced pressure. After 30 minutes on the vacuum pump, fresh was added followed by a solution of 3,4-dihydro-2H-benzo[b][1,4]oxazine-7-carbonitrile (18 mg, 0.112 mmol) in dichloromethane (1.0 mL). Triethylamine (0.20 mL, 0.195 mmol) was added and the reaction stirred at room temperature for 1 hour. The solvent was removed and the residue was purified using mass trigger HPLC. MS m/z 450.1 (M+H)+; r.t.=1.835.

### Synthesis of Compound 316

### 6-methanesulfonyl-1-({3-[4-(trifluoromethyl)phenyl]imidazo[1,2-a]pyrazin-6-yl}carbonyl)-1,2,3,4-tetrahydroquinoline

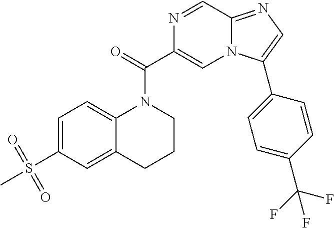 [SMILES: CS(=O)(=O)c1ccc2c(c1)CCCN2C(=O)c1cn2c(-c3ccc(C(F)(F)F)cc3)cnc2cn1]

The compound was synthesized using a procedure similar to one used for the synthesis of 128. MS m/z 501.1 (M+H)+; r.t.=2.050.

### Synthesis of Compound 317

### N-(5-methanesulfonylpyridin-2-yl)-N-methyl-3-[4-(trifluoromethyl)phenyl]imidazo[1,2-a]pyrazine-6-carboxamide

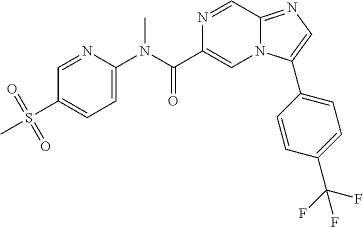 [SMILES: CN(C(=O)c1cn2c(-c3ccc(C(F)(F)F)cc3)cnc2cn1)c1ccc(S(C)(=O)=O)cn1]

The compound was synthesized using a procedure analogous to one use for synthesis of 72 (method II). ¹H NMR (400 MHz, MeOD) δ 9.05 (s, 1H), 8.76 (s, 1H), 8.73 (s, 1H), 8.24 (d, J=8.6 Hz, 1H), 8.15 (s, 1H), 7.96 (s, 4H), 7.55 (d, J=8.6 Hz, 1H), 3.66 (s, 3H), 3.20 (s, 3H). ESI-MS m/z 476 [M+1]. RT: 1.32 min.

### Synthesis of Compound 318

### methyl N-[4-(6-{[methyl(5-methylpyridin-2-yl)amino]methyl}imidazo[1,2-a]pyrazin-3-yl)phenyl]carbamate

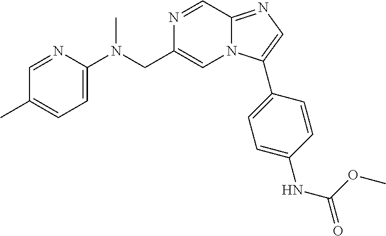

The compound was synthesized analogous to I-15 followed by Suzuki coupling protocol described in 228. MS m/z 403.2 (M+H)+; r.t.=1.055.

### Synthesis of Compound 319

### 3-(1H-indol-2-yl)-N-[6-(1H-indol-2-yl)pyridin-3-yl]-N-methylimidazo[1,2-a]pyrazine-6-carboxamide

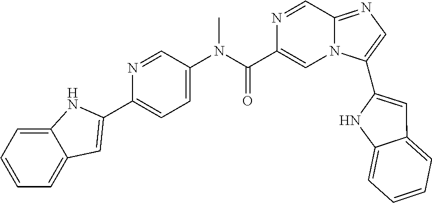 [SMILES: CN(C(=O)c1cn2c(-c3cc4ccccc4[nH]3)cnc2cn1)c1ccc(-c2cc3ccccc3[nH]2)nc1]

The compound was synthesized using I-19 and an appropriate boronic acid/ester using a procedure similar Suzuki coupling protocol described in 228. M/Z=484.1 (M+1), r.t=1.80 mins.

### Synthesis of Compound 320

### N-(6-chloropyridin-3-yl)-N-methyl-3-(3-methyl-1H-indazol-5-yl)imidazo[1,2-a]pyrazine-6-carboxamide

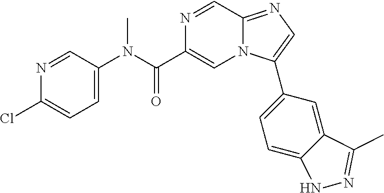 [SMILES: Cc1n[nH]c2ccc(-c3cnc4cnc(C(=O)N(C)c5ccc(Cl)nc5)cn34)cc12]

The compound was synthesized using I-57 and an appropriate boronic acid/ester using a procedure similar Suzuki coupling protocol described in 228. ¹H NMR (400 MHz, MeOD) δ 8.95 (s, 1H), 8.11-8.07 (m, 1H), 7.71 (d, J=8.2 Hz, 2H), 7.45 (s, 3H), 7.17-7.08 (m, 2H), 3.54 (s, 3H), 2.66 (s, 3H). ESI-MS m/z 418 [M+1]. RT: 1.18 min.

### Synthesis of Compound 321

### N-methyl-N-[5-(morpholin-4-yl)pyridin-2-yl]-3-[4-(trifluoromethyl)phenyl]imidazo[1,2-a]pyrazine-6-carboxamide

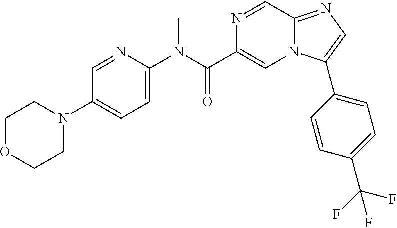 [SMILES: CN(C(=O)c1cn2c(-c3ccc(C(F)(F)F)cc3)cnc2cn1)c1ccc(N2CCOCC2)cn1]

The compound was synthesized using a procedure analogous to one use for synthesis of 72 (method II). M/Z=483.2 (M+1), r.t=1.37 mins.

### Synthesis of Compound 322

### 4-fluoro-N-methyl-N-{3-[4-(trifluoromethyl)phenyl]imidazo[1,2-a]pyrazin-6-yl}piperidine-1-carboxamide

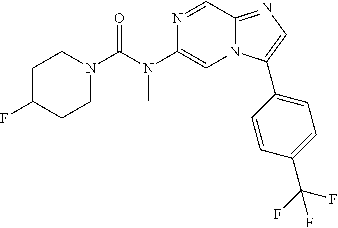 [SMILES: CN(C(=O)N1CCC(F)CC1)c1cn2c(-c3ccc(C(F)(F)F)cc3)cnc2cn1]

The compound was synthesized using similar to synthesis of 24. M/Z=422.2 (M+1), r.t=1.65 mins.

### Synthesis of Compound 323

### N-(4-cyanophenyl)-3-(6-methanesulfonamidopyridin-3-yl)-N-methylimidazo[1,2-a]pyrazine-6-carboxamide

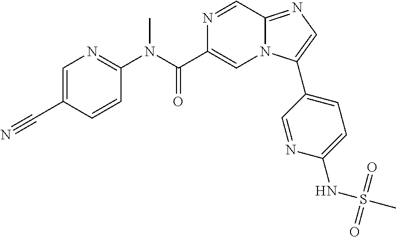 [SMILES: CN(C(=O)c1cn2c(-c3ccc(NS(C)(=O)=O)nc3)cnc2cn1)c1ccc(C#N)cn1]

The compound was synthesized using I-59 and an appropriate boronic acid/ester using a procedure similar Suzuki coupling protocol described in 228. MS m/z 448.1 (M+H)+; r.t.=1.164.

### Synthesis of compound 324

### 3-{4-[(1-amino-2-methylpropan-2-yl)amino]phenyl}-N-(4-cyanophenyl)-N-methylimidazo[1,2-a]pyrazine-6-carboxamide

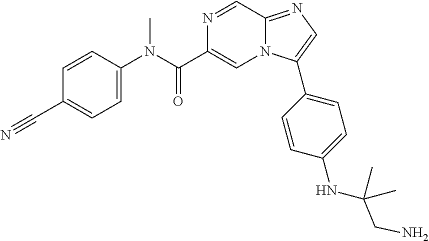 [SMILES: CN(C(=O)c1cn2c(-c3ccc(NC(C)(C)CN)cc3)cnc2cn1)c1ccc(C#N)cc1]

The compound was synthesized using I-59 and an appropriate boronic acid/ester using a procedure similar Suzuki coupling protocol described in 228. ¹H NMR (400 MHz, MeOD) δ 8.91-8.52 (m, 3H), 8.06-7.79 (m, 2H), 7.74-7.65 (m, 1H), 7.62-7.51 (m, 1H), 7.50-7.34 (m, 2H), 7.07-6.86 (m, 2H), 3.56 (s, 3H), 3.20-3.07 (m, 2H), 1.44 (s, 6H). ESI-MS m/z 440 [M+1]. RT: 1.09 min.

### Synthesis of Compound 325

### 3-(1-benzofuran-5-yl)-N-(6-chloropyridin-3-yl)-N-methylimidazo[1,2-a]pyrazine-6-carboxamide

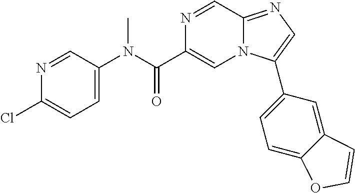 [SMILES: CN(C(=O)c1cn2c(-c3ccc4occc4c3)cnc2cn1)c1ccc(Cl)nc1]

The compound was synthesized using I-57 and an appropriate boronic acid/ester using a procedure similar Suzuki coupling protocol described in 228. M/Z=404.1 (M+1), r.t=1.49 mins.

### Synthesis of Compound 326

### N-methyl-N-(pyridin-3-yl)-3-[4-(trifluoromethyl)phenyl]imidazo[1,2-a]pyrazine-6-carboxamide

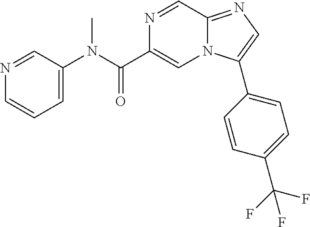 [SMILES: CN(C(=O)c1cn2c(-c3ccc(C(F)(F)F)cc3)cnc2cn1)c1cccnc1]

The compound was synthesized using a procedure analogous to one use for synthesis of 72 (method II) using I-41. MS m/z 398.1 (M+H)+; r.t.=1.136.

### Synthesis of Compound 327

### N-methyl-N-(5-methylpyrazin-2-yl)-3-[4-(trifluoromethyl)phenyl]imidazo[1,2-a]pyrazine-6-carboxamide

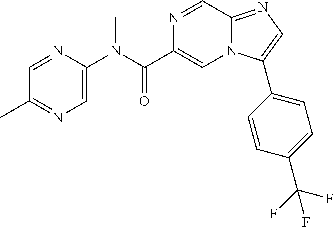 [SMILES: Cc1cnc(N(C)C(=O)c2cn3c(-c4ccc(C(F)(F)F)cc4)cnc3cn2)cn1]

The compound was synthesized using a procedure analogous to one use for synthesis of 72 (method II). M/Z=413.1 (M+1), r.t=1.60 mins.

### Synthesis of Compound 328

### N-methyl-N-(5-methylpyrazin-2-yl)-3-[4-(trifluoromethyl)phenyl]imidazo[1,2-a]pyrazine-6-carboxamide

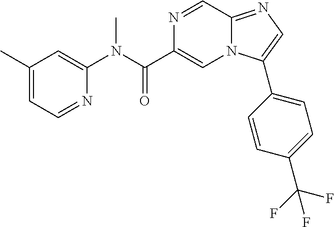 [SMILES: Cc1ccnc(N(C)C(=O)c2cn3c(-c4ccc(C(F)(F)F)cc4)cnc3cn2)c1]

The compound was synthesized using a procedure analogous to one use for synthesis of 72 (method II). M/Z=412.1 (M+1), r.t=1.53 mins.

### Synthesis of Compound 329

### N-methyl-3-[4-(trifluoromethyl)phenyl]-N-[2-(trifluoromethyl)pyridin-4-yl]imidazo[1,2-a]pyrazine-6-carboxamide

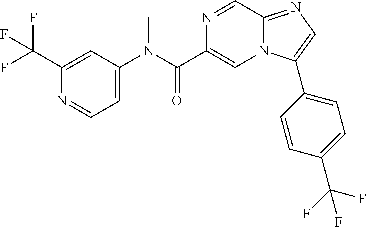 [SMILES: CN(C(=O)c1cn2c(-c3ccc(C(F)(F)F)cc3)cnc2cn1)c1ccnc(C(F)(F)F)c1]

The compound was synthesized using a procedure analogous to one use for synthesis of 72 (method II) using I-54. MS m/z 466.1 (M+H)+; r.t.=1.639.

### Synthesis of Compound 330

### N-(6-chloropyridin-3-yl)-N-methyl-3-[4-(1H-pyrazol-1-yl)phenyl]imidazo[1,2-a]pyrazine-6-carboxamide

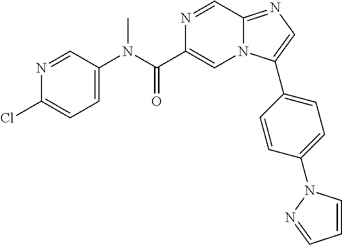 [SMILES: CN(C(=O)c1cn2c(-c3ccc(-n4cccn4)cc3)cnc2cn1)c1ccc(Cl)nc1]

The compound was synthesized using I-57 and an appropriate boronic acid/ester using a procedure similar Suzuki coupling protocol described in 228. ¹H NMR (400 MHz, MeOD) δ 8.99 (s, 1H), 8.86-8.60 (m, 2H), 8.40 (s, 1H), 8.35-8.21 (m, 1H), 8.20-7.96 (m, 3H), 7.92-7.67 (m, 3H), 7.53-7.35 (m, 1H), 6.61 (s, 1H), 3.54 (s, 3H). ESI-MS m/z 430 [M+1]. RT: 1.43 min.

### Synthesis of Compound 331

### 5-{6-[(4-cyanophenyl)(methyl)carbamoyl]imidazo[1,2-a]pyrazin-3-yl}-N-methylpyridine-2-carboxamide

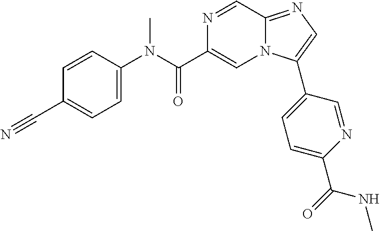 [SMILES: CNC(=O)c1ccc(-c2cnc3cnc(C(=O)N(C)c4ccc(C#N)cc4)cn23)cn1]

The compound was synthesized using I-59 and an appropriate boronic acid/ester using a procedure similar Suzuki coupling protocol described in 228. M/Z=412.2 (M+1), r.t=1.16 mins.

### Synthesis of Compound 332

### N-(6-chloropyridin-3-yl)-3-[3-fluoro-4-(trifluoromethyl)phenyl]-N-methylimidazo[1,2-a]pyrazine-6-carboxamide

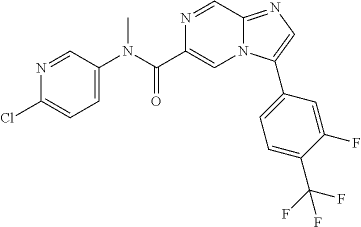 [SMILES: CN(C(=O)c1cn2c(-c3ccc(C(F)(F)F)c(F)c3)cnc2cn1)c1ccc(Cl)nc1]

The compound was synthesized using I-57 and an appropriate boronic acid/ester using a procedure similar Suzuki coupling protocol described in 228. M/Z=450.1 (M+1), r.t=1.68 mins.

### Synthesis of Compound 333

### N-(4-cyanophenyl)-3-(2-methoxypyrimidin-5-yl)-N-methylimidazo[1,2-a]pyrazine-6-carboxamide

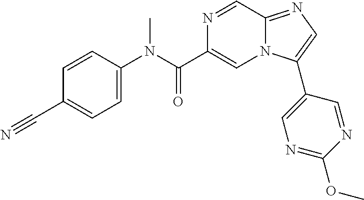 [SMILES: COc1ncc(-c2cnc3cnc(C(=O)N(C)c4ccc(C#N)cc4)cn23)cn1]

The compound was synthesized using I-59 and an appropriate boronic acid/ester using a procedure similar Suzuki coupling protocol described in 228. M/Z=386.2 (M+1), r.t=1.17 mins.

### Synthesis of compound 334

### N-(4-ethylpyridin-2-yl)-3-[4-(trifluoromethyl)phenyl]imidazo[1,2-a]pyrazine-6-carboxamide

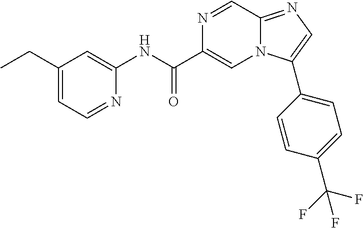 [SMILES: CCc1ccnc(NC(=O)c2cn3c(-c4ccc(C(F)(F)F)cc4)cnc3cn2)c1]

The compound was synthesized using a procedure analogous to one use for synthesis of 72 (method II). ¹H NMR (300 MHz, DMSO-d₆) ppm 10.27 (s, 1H) 9.33 (d, J=1.47 Hz, 1H) 9.15 (d, J=1.47 Hz, 1H) 8.35 (s, 1H) 8.29 (d, J=4.98 Hz, 1H) 8.16 (s, 1H) 7.96-8.11 (m, 4H) 7.07-7.15 (m, 1H) 2.69 (q, J=7.33 Hz, 2H) 1.22 (t, J=7.62 Hz, 3H); MS (ESI) for C₂₁H₁₆F₃N₅O: 412 (M+H⁺).

### Synthesis of Compound 335

### N-(4-cyanophenyl)-3-(6-methoxypyrazin-2-yl)-N-methylimidazo[1,2-a]pyrazine-6-carboxamide

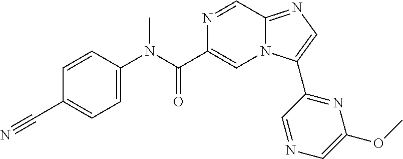 [SMILES: COc1cncc(-c2cnc3cnc(C(=O)N(C)c4ccc(C#N)cc4)cn23)n1]

The compound was synthesized using I-59 and an appropriate boronic acid/ester using a procedure similar Suzuki coupling protocol described in 228. M/Z=386.2 (M+1), r.t=1.24 mins.

### Synthesis of Compound 336

### N-(6-chloropyridin-3-yl)-3-(6-methoxypyridin-3-yl)-N-methylimidazo[1,2-a]pyrazine-6-carboxamide

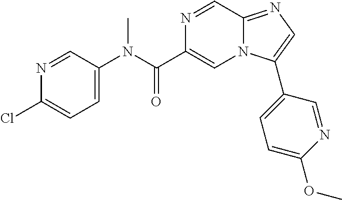 [SMILES: COc1ccc(-c2cnc3cnc(C(=O)N(C)c4ccc(Cl)nc4)cn23)cn1]

The compound was synthesized using I-57 and an appropriate boronic acid/ester using a procedure similar Suzuki coupling protocol described in 228. M/Z=395.1 (M+1), r.t=1.27 mins.

### Synthesis of Compound 337

### N-methyl-3-[4-(trifluoromethyl)phenyl]-N-[5-(trifluoromethyl)pyridin-2-yl]imidazo[1,2-a]pyrazine-6-carboxamide

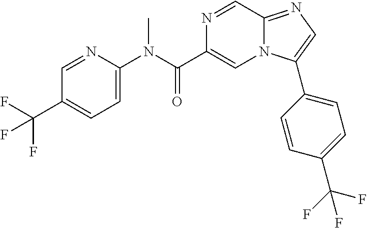 [SMILES: CN(C(=O)c1cn2c(-c3ccc(C(F)(F)F)cc3)cnc2cn1)c1ccc(C(F)(F)F)cn1]

The compound was synthesized using a procedure analogous to one use for synthesis of 72 (method II). M/Z=507.23 (M+1), r.t=1.24 mins.

### Synthesis of Compound 338

### N-methyl-3-[4-(trifluoromethyl)phenyl]-N-[5-(trifluoromethyl)pyridin-2-yl]imidazo[1,2-a]pyrazine-6-carboxamide

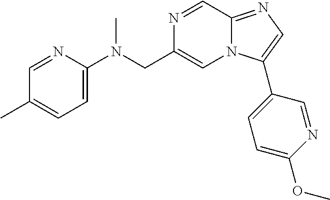

The compound was synthesized analogous to I-15 followed by Suzuki coupling protocol described in 228. MS m/z 361.2 (M+H)+; r.t.=1.074.

### Synthesis of Compound 339

### N-(4-cyanophenyl)-N-methyl-3-[4-(3-methyl-1,2,4-oxadiazol-5-yl)phenyl]imidazo[1,2-a]pyrazine-6-carboxamide

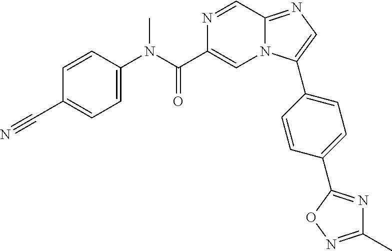 [SMILES: Cc1noc(-c2ccc(-c3cnc4cnc(C(=O)N(C)c5ccc(C#N)cc5)cn34)cc2)n1]

The compound was synthesized using I-59 and an appropriate boronic acid/ester using a procedure similar Suzuki coupling protocol described in 228. M/Z=436.1 (M+1), r.t=1.49 mins.

### Synthesis of Compound 340

### N-(6-chloropyridin-3-yl)-N-methyl-3-[4-(morpholin-4-yl)phenyl]imidazo[1,2-a]pyrazine-6-carboxamide

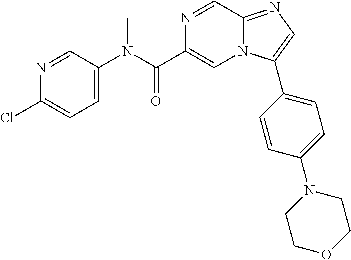 [SMILES: CN(C(=O)c1cn2c(-c3ccc(N4CCOCC4)cc3)cnc2cn1)c1ccc(Cl)nc1]

The compound was synthesized using I-57 and an appropriate boronic acid/ester using a procedure similar Suzuki coupling protocol described in 228. M/Z=449.1 (M+1), r.t=1.36 mins.

### Synthesis of Compound 341

### N,3-bis(4-cyanophenyl)-N-methyl-2-(trifluoromethyl)imidazo[1,2-a]pyrazine-6-carboxamide

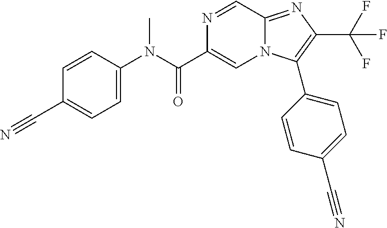 [SMILES: CN(C(=O)c1cn2c(-c3ccc(C#N)cc3)c(C(F)(F)F)nc2cn1)c1ccc(C#N)cc1]

The compound was synthesized using I-59 and an appropriate boronic acid/ester using a procedure similar Suzuki coupling protocol described in 228. MS m/z 447.0 (M+H)+; r.t.=1.743.

### Synthesis of Compound 342

### N-(2,6-dichloropyridin-3-yl)-N-methyl-3-[4-(trifluoromethyl)phenyl]imidazo[1,2-a]pyrazine-6-carboxamide

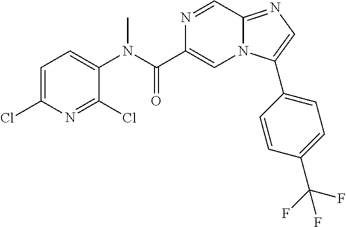 [SMILES: CN(C(=O)c1cn2c(-c3ccc(C(F)(F)F)cc3)cnc2cn1)c1ccc(Cl)nc1Cl]

The compound was synthesized using a procedure analogous to one use for synthesis of 72 (method II) using I-43. MS m/z 466.0 (M+H)+; r.t.=1.860.

### Synthesis of Compound 343

### N-(6-chloropyridin-3-yl)-3-[4-(cyclopropylcarbamoyl)phenyl]-N-methylimidazo[1,2-a]pyrazine-6-carboxamide

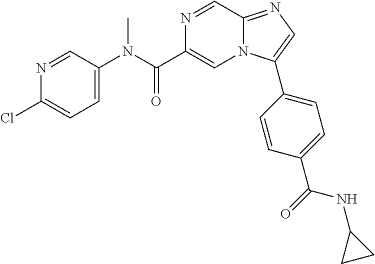 [SMILES: CN(C(=O)c1cn2c(-c3ccc(C(=O)NC4CC4)cc3)cnc2cn1)c1ccc(Cl)nc1]

The compound was synthesized using I-57 and an appropriate boronic acid/ester using a procedure similar Suzuki coupling protocol described in 228. M/Z=447.1 (M+1), r.t=1.25 mins.

### Synthesis of Compound 344

### N-(4-cyanophenyl)-3-[2-(dimethylamino)pyrimidin-5-yl]-N-methylimidazo[1,2-a]pyrazine-6-carboxamide

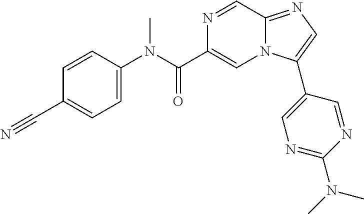 [SMILES: CN(C)c1ncc(-c2cnc3cnc(C(=O)N(C)c4ccc(C#N)cc4)cn23)cn1]

The compound was synthesized using I-59 and an appropriate boronic acid/ester using a procedure similar Suzuki coupling protocol described in 228. M/Z=399.2 (M+1), r.t=1.23 mins.

### Synthesis of Compound 345

### N-methyl-N-(6-methylpyridin-2-yl)-3-[4-(trifluoromethyl)phenyl]imidazo[1,2-a]pyrazine-6-carboxamide

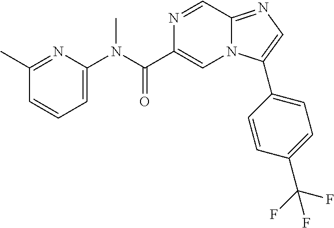 [SMILES: Cc1cccc(N(C)C(=O)c2cn3c(-c4ccc(C(F)(F)F)cc4)cnc3cn2)n1]

The compound was synthesized using a procedure analogous to one use for synthesis of 72 (method II). M/Z=412.2 (M+1), r.t=1.51 mins.

### Synthesis of Compound 346

### N-[2-(difluoromethoxy)phenyl]-N-methyl-3-[4-(trifluoromethyl)phenyl]imidazo[1,2-a]pyrazine-6-carboxamide

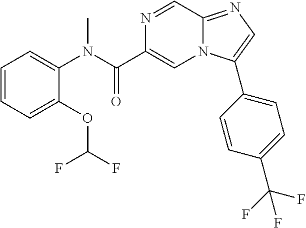 [SMILES: CN(C(=O)c1cn2c(-c3ccc(C(F)(F)F)cc3)cnc2cn1)c1ccccc1OC(F)F]

compound was synthesized using a procedure analogous to one use for synthesis of 72 (method II). MS m/z 463.1 (M+H)+; r.t.=1.804.

### Synthesis of Compound 347

### 4-cyano-N-methyl-N-{3-[4-(trifluoromethyl)phenyl]imidazo[1,2-a]pyrazin-6-yl}benzene-1-sulfonamide

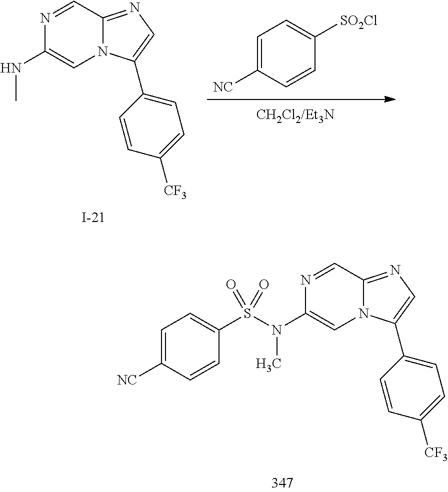

A solution of NHMe core I-21 (1.0 equiv.) in CH2Cl2 was treated with sulfonyl chloride (1.5 equiv.) and Et3N (5.0 equiv.) at room temperature. The resulting mixture was allowed to stir at 60° C. overnight. The reaction mixture was concentrated, redissolved in 10% MeOH/DMSO, and purified by mass-triggered HPLC. ¹H NMR (400 MHz, CDCl3) δ 8.88 (d, J=1.3, 1H), 8.55 (d, J=1.3, 1H), 8.02 (s, 1H), 7.86 (d, J=8.2, 2H), 7.82-7.71 (m, 6H), 3.25 (s, 3H). M/Z=458.1 (M+1), r.t=2.11 mins.

### Synthesis of Compound 348

### N-methyl-3-[4-(trifluoromethyl)phenyl]-N-[4-(trifluoromethyl)pyridin-2-yl]imidazo[1,2-a]pyrazine-6-carboxamide

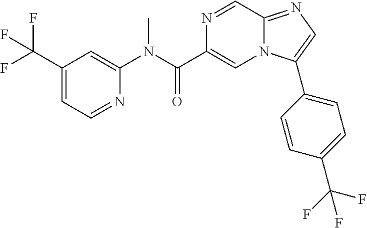 [SMILES: CN(C(=O)c1cn2c(-c3ccc(C(F)(F)F)cc3)cnc2cn1)c1cc(C(F)(F)F)ccn1]

compound was synthesized using a procedure analogous to one use for synthesis of 72 (method II). M/Z=466.10 (M+1), r.t=1.98 mins.

### Synthesis of Compound 349

### N-(6-chloropyridin-3-yl)-3-(1H-indazol-5-yl)-N-methylimidazo[1,2-a]pyrazine-6-carboxamide

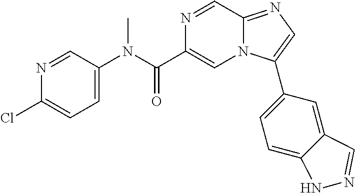 [SMILES: CN(C(=O)c1cn2c(-c3ccc4[nH]ncc4c3)cnc2cn1)c1ccc(Cl)nc1]

The compound was synthesized using I-57 and an appropriate boronic acid/ester using a procedure similar Suzuki coupling protocol described in 228. ¹H NMR (400 MHz, MeOD) δ 8.99-8.94 (m, 1H), 8.89-8.84 (m, 1H), 8.82-8.73 (m, 1H), 8.25 (s, 3H), 8.06 (s, 1H), 7.80 (s, 3H), 3.56 (s, 3H). ESI-MS m/z 404 [M+1]. RT: 1.16 min.

### Synthesis of Compound 350

### 2,2-dimethyl-4-({3-[4-(trifluoromethyl)phenyl]imidazo[1,2-a]pyrazin-6-yl}carbonyl)-3,4-dihydro-2H-1,4-benzoxazine-7-carbonitrile

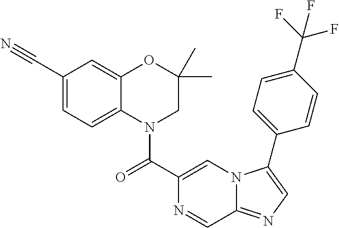 [SMILES: CC1(C)CN(C(=O)c2cn3c(-c4ccc(C(F)(F)F)cc4)cnc3cn2)c2ccc(C#N)cc2O1]

The compound was synthesized using I-56 a procedure analogous to one use for synthesis of 72 (method II). MS m/z 478.1 (M+H)+; r.t.=1.997.

### Synthesis of Compound 351

### N-(4-cyanophenyl)-3-(6-fluoropyridin-2-yl)-N-methylimidazo[1,2-a]pyrazine-6-carboxamide

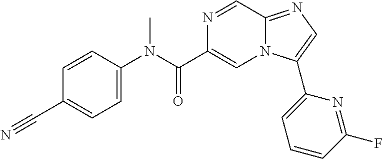 [SMILES: CN(C(=O)c1cn2c(-c3cccc(F)n3)cnc2cn1)c1ccc(C#N)cc1]

The compound was synthesized using I-59 and an appropriate boronic acid/ester using a procedure similar Suzuki coupling protocol described in 228. M/Z=373.10 (M+1), r.t=1.31 mins.

### Synthesis of Compound 352

### 3-[4-(1-carbamoyl-1-methylethoxy)phenyl]-N-(4-cyanophenyl)-N-methylimidazo[1,2-a]pyrazine-6-carboxamide

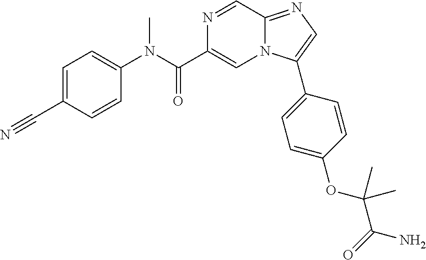 [SMILES: CN(C(=O)c1cn2c(-c3ccc(OC(C)(C)C(N)=O)cc3)cnc2cn1)c1ccc(C#N)cc1]

The compound was synthesized using I-59 and I-67 using a procedure similar Suzuki coupling protocol described in 228. ¹H NMR (400 MHz, MeOD) δ 8.90-8.68 (m, 2H), 8.06-7.95 (m, 1H), 7.68 (d, J=8.5 Hz, 2H), 7.55 (d, J=8.7 Hz, 2H), 7.41 (d, J=8.5 Hz, 2H), 7.19 (d, J=8.7 Hz, 2H), 3.56 (s, 3H), 1.62 (s, 6H). ESI-MS m/z 455 [M+1]. RT: 1.29 min.

### Synthesis of Compound 353

### N-(5-methoxypyridin-2-yl)-N-methyl-3-[4-(trifluoromethyl)phenyl]imidazo[1,2-a]pyrazine-6-carboxamide

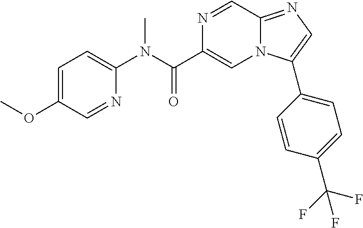 [SMILES: COc1ccc(N(C)C(=O)c2cn3c(-c4ccc(C(F)(F)F)cc4)cnc3cn2)nc1]

The compound was synthesized using a procedure analogous to one use for synthesis of 72 (method II). ¹H NMR (400 MHz, MeOD) δ 9.18-9.13 (m, 1H), 8.93-8.88 (m, 1H), 8.72-8.66 (m, 1H), 8.24-8.17 (m, 1H), 8.11 (s, 1H), 7.93 (s, 2H), 7.43-7.27 (m, 3H), 3.84 (s, 3H), 3.54 (s, 3H). ESI-MS m/z 428 [M+1]. RT: 1.55 min.

### Synthesis of Compound 354

### 3-(3-chloropyridin-2-yl)-N-(4-cyanophenyl)-N-methylimidazo[1,2-a]pyrazine-6-carboxamide

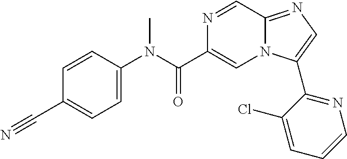 [SMILES: CN(C(=O)c1cn2c(-c3ncccc3Cl)cnc2cn1)c1ccc(C#N)cc1]

The compound was synthesized using I-59 and an appropriate boronic acid/ester using a procedure similar Suzuki coupling protocol described in 228. M/Z=389 (M+1), r.t=1.28 mins.

### Synthesis of Compound 355

### N-(5-chloropyridin-3-yl)-N-methyl-3-[4-(trifluoromethyl)phenyl]imidazo[1,2-a]pyrazine-6-carboxamide

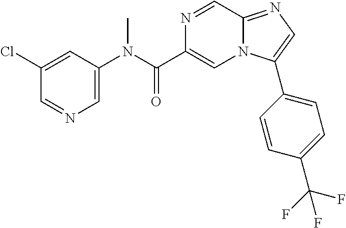 [SMILES: CN(C(=O)c1cn2c(-c3ccc(C(F)(F)F)cc3)cnc2cn1)c1cncc(Cl)c1]

The compound was synthesized using a procedure analogous to one use for synthesis of 72 (method II) using I-49. MS m/z 432.1 (M+H)+; r.t.=1.711.

### Synthesis of Compound 356

### N-methyl-N-(1,3-thiazol-2-yl)-3-[4-(trifluoromethyl)phenyl]imidazo[1,2-a]pyrazine-6-carboxamide

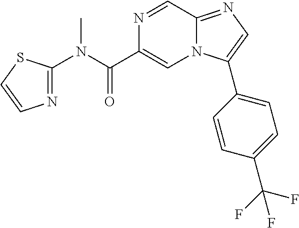 [SMILES: CN(C(=O)c1cn2c(-c3ccc(C(F)(F)F)cc3)cnc2cn1)c1nccs1]

The compound was synthesized using a procedure analogous to one use for synthesis of 72 (method III). MS m/z 404.1 (M+H)+; r.t.=2.190.

### Synthesis of Compound 357

### N-(6-chloropyridin-3-yl)-3-[6-(dimethylamino)pyridin-3-yl]-N-methylimidazo[1,2-a]pyrazine-6-carboxamide

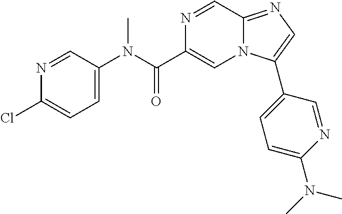 [SMILES: CN(C)c1ccc(-c2cnc3cnc(C(=O)N(C)c4ccc(Cl)nc4)cn23)cn1]

The compound was synthesized using I-57 and an appropriate boronic acid/ester using a procedure similar Suzuki coupling protocol described in 228. M/Z=408 (M+1), r.t=0.926 mins.

### Synthesis of Compound 358

### -(4-methoxypyridin-2-yl)-N-methyl-3-[4-(trifluoromethyl)phenyl]imidazo[1,2-a]pyrazine-6-carboxamide

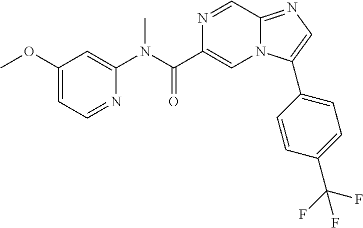 [SMILES: COc1ccnc(N(C)C(=O)c2cn3c(-c4ccc(C(F)(F)F)cc4)cnc3cn2)c1]

The compound was synthesized using a procedure similar to one used for the synthesis of 128. M/Z=428.1 (M+1), r.t=1.52 mins.

### Synthesis of Compound 359

### N-{6-[(2S)-2-(methoxymethyl)pyrrolidin-1-yl]pyridin-3-yl}-N-methyl-3-[4-(trifluoromethyl)phenyl]imidazo[1,2-a]pyrazine-6-carboxamide

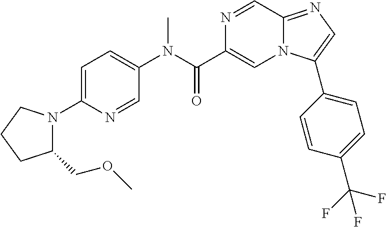 [SMILES: COC[C@@H]1CCCN1c1ccc(N(C)C(=O)c2cn3c(-c4ccc(C(F)(F)F)cc4)cnc3cn2)cn1]

The compound was synthesized using a procedure analogous to one use for synthesis of 72 (method II). M/Z=511.3 (M+1), r.t=1.43 mins.

### Synthesis of Compound 360

### N-(6-chloropyridin-3-yl)-3-(2,3-dihydro-1,4-benzodioxin-6-yl)-N-methylimidazo[1,2-a]pyrazine-6-carboxamide

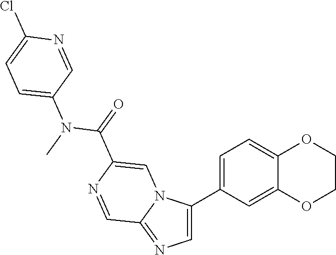 [SMILES: CN(C(=O)c1cn2c(-c3ccc4c(c3)OCCO4)cnc2cn1)c1ccc(Cl)nc1]

The compound was synthesized using I-57 and an appropriate boronic acid/ester using a procedure similar Suzuki coupling protocol described in 228. M/Z=422.10 (M+1), r.t=1.43 mins.

### Synthesis of Compound 361

### 3-(4-acetamidophenyl)-N-methyl-N-(5-methylpyridin-2-yl)imidazo[1,2-a]pyrazine-6-carboxamide

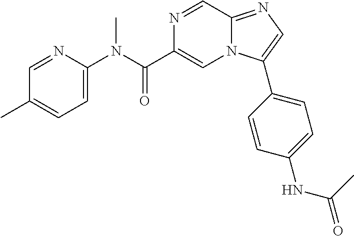 [SMILES: CC(=O)Nc1ccc(-c2cnc3cnc(C(=O)N(C)c4ccc(C)cn4)cn23)cc1]

The compound was synthesized using I-78 and an appropriate boronic acid/ester using a procedure similar Suzuki coupling protocol described in 228. M/Z=401.2 (M+1), r.t=0.98 mins.

### Synthesis of Compound 362

### -[(4-cyanophenyl)methyl]-3-[4-(trifluoromethyl)phenyl]imidazo[1,2-a]pyrazine-6-carboxamide

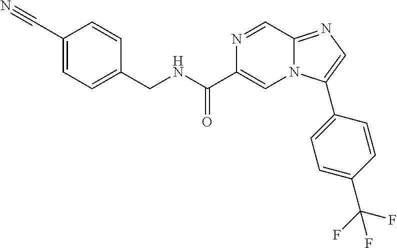 [SMILES: N#Cc1ccc(CNC(=O)c2cn3c(-c4ccc(C(F)(F)F)cc4)cnc3cn2)cc1]

The compound was synthesized using I-59 and an appropriate boronic acid/ester using a procedure similar Suzuki coupling protocol described in 228. ¹H NMR (300 MHz, CDCl₃) 9.07-9.14 (dd, J=1.5 Hz, 2H), 8.39 (t, broad, 1H), 8.02 (s, 1H), 7.46-7.86 (m, J=8.4 Hz, 8H), 4.74-4.76 (d, J=6.0 Hz, 2H). ESI-MS m/z 422 [M+H]⁺.

### Synthesis of Compound 363

### N-(4-cyanophenyl)-N-methyl-3-[4-(propane-2-sulfonamido)phenyl]imidazo[1,2-a]pyrazine-6-carboxamide

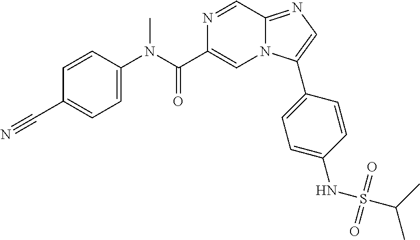 [SMILES: CC(C)S(=O)(=O)Nc1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. M/Z=475.1 (M+1), r.t=1.50 mins.

### Synthesis of Compound 364

### N-(6-chloropyridin-3-yl)-N-methyl-3-[2-(propan-2-yl)-1,3-thiazol-4-yl]imidazo[1,2-a]pyrazine-6-carboxamide

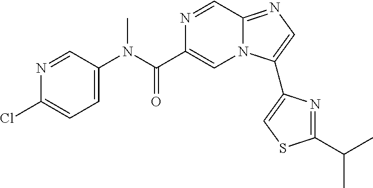 [SMILES: CC(C)c1nc(-c2cnc3cnc(C(=O)N(C)c4ccc(Cl)nc4)cn23)cs1]

The compound was synthesized using I-57 and an appropriate boronic acid/ester using a procedure similar Suzuki coupling protocol described in 228. M/Z=413.1 (M+1), r.t=1.60 mins.

### Synthesis of Compound 365

### N-methyl-N-[6-(1H-1,2,4-triazol-1-yl)pyridin-3-yl]-3-[4-(trifluoromethyl)phenyl]imidazo[1,2-a]pyrazine-6-carboxamide

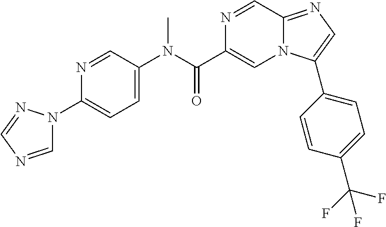 [SMILES: CN(C(=O)c1cn2c(-c3ccc(C(F)(F)F)cc3)cnc2cn1)c1ccc(-n2cncn2)nc1]

The compound was synthesized using a procedure analogous to one use for synthesis of 72 (method II) using I-45. MS m/z 465.1 (M+H)+; r.t.=1.541.

### Synthesis of Compound 366

### N-methyl-4-(trifluoromethoxy)-N-{3-[4-(trifluoromethyl)phenyl]imidazo[1,2-a]pyrazin-6-yl}benzamide

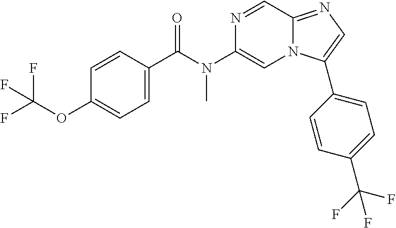 [SMILES: CN(C(=O)c1ccc(OC(F)(F)F)cc1)c1cn2c(-c3ccc(C(F)(F)F)cc3)cnc2cn1]

The compound was synthesized using a procedure analogous to one use for synthesis of 24. 1H NMR (400 MHz, CDCl₃) δ 9.08 (s, 1H), 7.86 (s, 1H), 7.82 (s, 1H), 7.64 (d, J=8.0, 2H), 7.42 (d, J=8.7, 2H), 7.14 (d, J=8.0, 4H), 3.51 (s, 3H). HRMS calcd for C₂₂H₁₅F₆N₄O₂ [M+H]+481.1; found 481.2.

### Synthesis of Compound 367

### N-(6-chloropyridin-3-yl)-N-methyl-3-[6-(5-methyl-1,3,4-oxadiazol-2-yl)pyridin-3-yl]imidazo[1,2-a]pyrazine-6-carboxamide

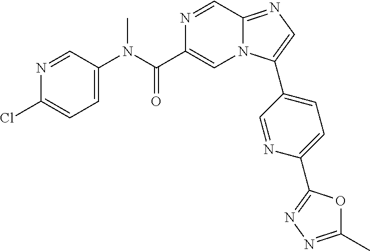 [SMILES: Cc1nnc(-c2ccc(-c3cnc4cnc(C(=O)N(C)c5ccc(Cl)nc5)cn34)cn2)o1]

The compound was synthesized using I-57 and an appropriate boronic acid/ester using a procedure similar Suzuki coupling protocol described in 228. M/Z=446.10 (M), r.t=1.46 mins.

### Synthesis of Compound 368

### N-(1-ethyl-1H-pyrazol-5-yl)-3-[4-(trifluoromethyl)phenyl]imidazo[1,2-a]pyrazine-6-carboxamide

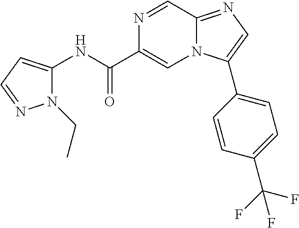 [SMILES: CCn1nccc1NC(=O)c1cn2c(-c3ccc(C(F)(F)F)cc3)cnc2cn1]

The compound was synthesized using a procedure analogous to one use for synthesis of 72 (method II). ¹H NMR (300 MHz, DMSO-d₆) ppm 1.31 (t, J=7.18 Hz, 3H), 4.04 (q, J=7.33 Hz, 3H), 6.24 (d, J=1.76 Hz, 1H), 7.43 (d, J=1.76 Hz, 1H), 7.94-8.11 (m, 4H), 8.33 (s, 1H), 9.09 (d, J=1.46 Hz, 1H), 9.31 (d, J=1.47 Hz, 1H). ESI-MS m/z 401 [M+H]⁺.

### Synthesis of Compound 369

### 3-(4-cyanophenyl)-N-methyl-N-[4-(trifluoromethyl)phenyl]imidazo[1,2-a]pyrazine-6-carboxamide

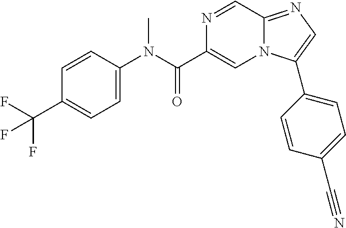 [SMILES: CN(C(=O)c1cn2c(-c3ccc(C#N)cc3)cnc2cn1)c1ccc(C(F)(F)F)cc1]

The compound was synthesized using a procedure analogous to one use for synthesis of 72 (method II). MS m/z 422.2 (M+H)+; r.t.=2.030.

### Synthesis of Compound 370

### N-benzyl-N-methyl-3-[4-(trifluoromethyl)phenyl]imidazo[1,2-a]pyrazine-6-carboxamide

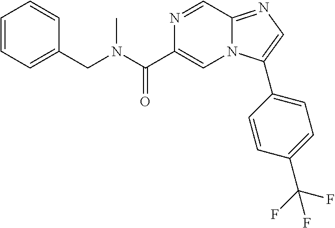 [SMILES: CN(Cc1ccccc1)C(=O)c1cn2c(-c3ccc(C(F)(F)F)cc3)cnc2cn1]

The compound was synthesized using a procedure analogous to one use for synthesis of 72 (method II). ¹H NMR (300 MHz, CDCl₃) 9.11 (d, J=11.72 Hz, 1H), 8.86 (d, J=7.62 Hz, 1H), 8.00 (br. s., 1H), 7.83 (br. s., 2H), 7.64-7.79 (m, 2H), 7.28-7.46 (m, 5H), 4.65-5.09 (m, 2H), 2.97-3.31 (m, 3H). ESI-MS m/z 411 [M+H]⁺.

### Synthesis of Compound 371

### 2-C-3-[4-(trifluoromethyl)phenyl]imidazo[1,2-a]pyrazine-6-pyridine-2,5-dicarboxamide

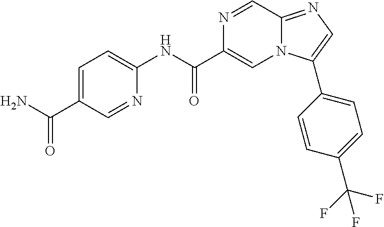 [SMILES: NC(=O)c1ccc(NC(=O)c2cn3c(-c4ccc(C(F)(F)F)cc4)cnc3cn2)nc1]

The compound was synthesized using a procedure analogous to one use for synthesis of 72 (method II). ¹H NMR (300 MHz, DMSO-d₆) ppm 10.52 (s, 1H) 9.33 (d, J=1.47 Hz, 1H) 9.16 (d, J=1.47 Hz, 1H) 8.88 (dd, J=2.05, 0.88 Hz, 1H) 8.27-8.40 (m, 3H) 8.04 (q, J=8.40 Hz, 5H) 7.53 (br. s., 1H); MS (ESI) for C₂₀H₁₃F₃N₆O₂: 427 (M+H⁺).

### Synthesis of Compound 372

### 3-[4-(2-aminoethoxyl)phenyl]-N-(4-cyanophenyl)-N-methylimidazo[1,2-a]pyrazine-6-carboxamide

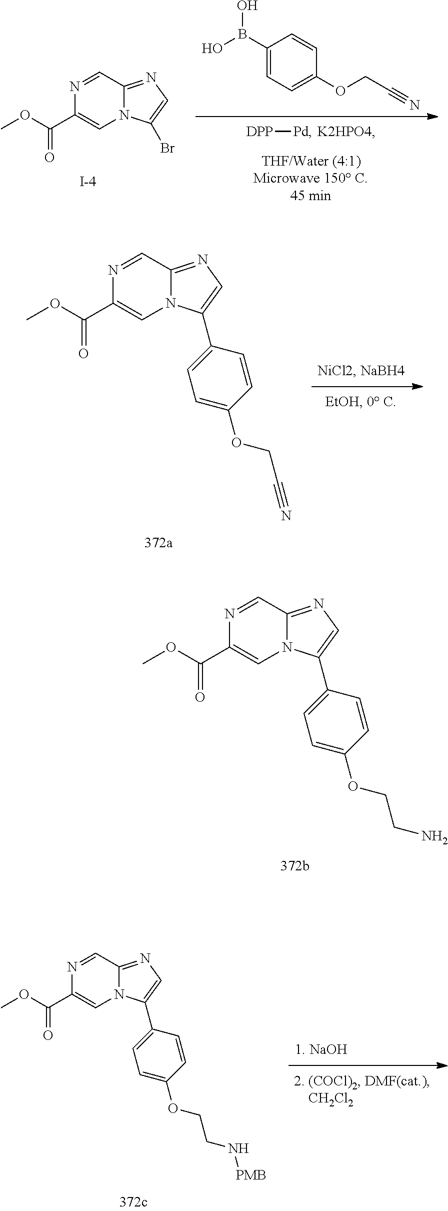

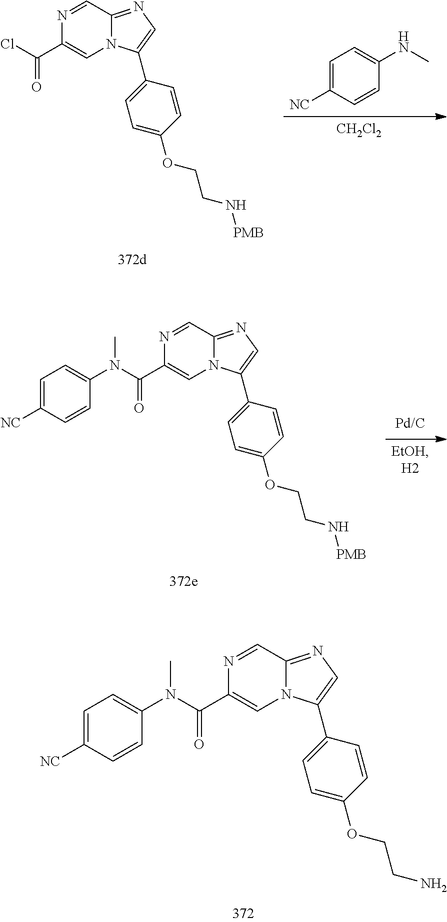

### Synthesis of methyl 3-(4-(cyanomethoxy)phenyl)imidazo[1,2-a]pyrazine-6-carboxylate (372a)

To a solution of methyl 3-bromoimidazo[1,2-a]pyrazine-6-carboxylate I-4 (0.15 g, 0.585 mmol, 1 eq), in THF/water (4:1) 10 mL was added the (4-(cyanomethoxy)phenyl)boronic acid (0.189 g, 0.732 mmol, 1.25 eq) followed by DPP-Pd (cat) and K₂HPO₄ (0.4 g, 2.34 mmol, 4 eq). The reaction vial was sealed and heated under microwave condition for 45 min at 150° C. The formation of the product peak was checked via LC/MS and the desired compound was isolated via flash column chromatography using ethyl acetate/hexane (0-100%). The isolated product was used for the next step.

### Synthesis of methyl 3-(4-(2-aminoethoxyl)phenyl)imidazo[1,2-a]pyrazine-6-carboxylate (372b)

To a solution of 3-(4-(cyanomethoxy)phenyl)imidazo[1,2-a]pyrazine-6-carboxylate (350 mg, 1.13 mmol, 1 eq), in EtOH (8 mL) was cooled down to 0° C. and was added NiCl₂ (220 mg, 1.70 mmol, 1.5 eq), followed by NaBH₄ (64 mg, 1.70mmols, 1.5 eq). The reaction mixture was stirred at 0° C. for 10 minutes and checked via LC/MS for the desired compound peak. The product was then isolated via flash column chromatography using ethyl acetate/hexane (0-100%). The isolated product was used for the next step.

### Synthesis of methyl 3-(4-(2-((4-methoxybenzyl)amino)ethoxy)phenyl)imidazo[1,2-a]pyrazine-6-carboxylate (372c)

To a solution of methyl 3-(4-(2-aminoethoxyl)phenyl)imidazo[1,2-a]pyrazine-6-carboxylate (260 mg, 0.832 mmol, 1 eq) in DCM was added the PMB-Cl (195 mg, 1.25 mmol, 1.5 eq), followed by DIEA (268 mg, 2.08 mmol, 2.5 eq). The reaction mixture was stirred at rt for 12 hrs and then was quenched with water, followed by extraction with ethyl acetate (3×15 mL). The desired compound was purified by using flash column chromatography by eluting with 0-100% ethyl acetate/hexane.

### Synthesis of 3-(4-(2-((4-methoxybenzyl)amino)ethoxy)phenyl)imidazo[1,2-a]pyrazine-6-carbonyl chloride (372d)

To a solution of methyl 3-(4-(2-((4-methoxybenzyl)amino)ethoxy)phenyl)imidazo[1,2-a]pyrazine-6-carboxylate (0.28 g, 0.647 mmol, 1 eq), was added 2 N NaOH (2 mL) and CH₃CN (6 mL). The reaction mixture was stirred at rt for 12 hrs and then the solvents were removed under vacuo. To the dry acid was then added the oxalyl chloride followed by DMF (cat) and the reaction mixture was stirred at room temperature overnight. Next day all the solvents were removed under vacuo and the crude reaction mixture was used for the next step.

### Synthesis of N-(4-cyanophenyl)-3-(4-(2-((4-methoxybenzyl)amino)ethoxy)phenyl)-N-methylimidazo[1,2-a]pyrazine-6-carboxamide (372e)

To a solution of 3-(4-(2-((4-methoxybenzyl)amino)ethoxy)phenyl)imidazo[1,2-a]pyrazine-6-carbonyl chloride in DCM was added the 4-(methylamino)benzonitrile (60 mg, 0.457 mmol, 1.25 eq) pre-dissolved (in DCM) drop wise over 10 minutes. The reaction mixture was stirred at room temperature overnight. The reaction was quenched with sodium carbonate solution and extracted the organics by using ethyl acetate (3×15 mL). The crude product was isolated and was used for the next step.

### Synthesis of 372

To a solution of N-(4-cyanophenyl)-3-(4-(2-((4-methoxybenzyl)amino)ethoxy)phenyl)-N-methylimidazo[1,2-a]pyrazine-6-carboxamide (60 mg, 0.112 mmol, 1 eq), dissolved in ethanol was added a few drops of AcOH. To this reaction mixture was then added the Pd/C (5%, 25 mg). The reaction mixture was kept under hydrogen by balloon for ½ hr. The desired product was isolated via flash column chromatography. ¹H NMR (400 MHz, MeOD) δ 8.12-8.07 (m, 1H), 7.68 (d, J=8.5 Hz, 2H), 7.61 (s, 1H), 7.42 (s, 2H), 7.28 (s, 3H), 6.95 (s, 2H), 4.36 (s, 2H), 4.05 (d, J=5.4 Hz, 2H), 3.34 (d, 3H). ESI-MS m/z 413 [M+1]. RT: 1.02 min.

### Synthesis of Compound 373

### 3-(6-acetamidopyridin-3-yl)-N-methyl-N-(5-methylpyridin-2-yl)imidazo[1,2-a]pyrazine-6-carboxamide

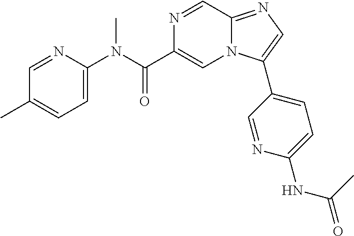 [SMILES: CC(=O)Nc1ccc(-c2cnc3cnc(C(=O)N(C)c4ccc(C)cn4)cn23)cn1]

The compound was synthesized using I-78 and an appropriate boronic acid/ester using a procedure similar Suzuki coupling protocol described in 228. M/Z=402.2 (M+1), r.t=0.92 mins.

### Synthesis of Compound 374

### 4-({3-[4-(trifluoromethyl)phenyl]imidazo[1,2-a]pyrazin-6-yl}carbonyl)morpholine

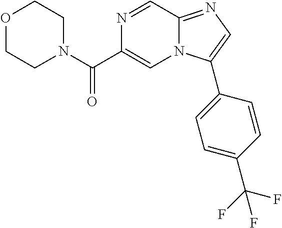 [SMILES: O=C(c1cn2c(-c3ccc(C(F)(F)F)cc3)cnc2cn1)N1CCOCC1]

The compound was synthesized using a procedure analogous to one use for synthesis of 72 (method II). ¹H NMR (300 MHz, CDCl₃) 9.10 (d, J=1.47 Hz, 1H), 8.87 (d, J=1.46 Hz, 1H), 8.02 (s, 1H), 7.79-7.89 (m, J=8.20 Hz, 2H), 7.67-7.79 (m, J=8.21 Hz, 2H), 3.64-3.73 (m, 4H), 2.40-2.60 (m, 4H). ESI-MS m/z 377 [M+H]⁺.

### Synthesis of Compound 375

### N-[4-(aminomethyl)phenyl]-N-methyl-3-[4-(trifluoromethyl)phenyl]imidazo[1,2-a]pyrazine-6-carboxamide

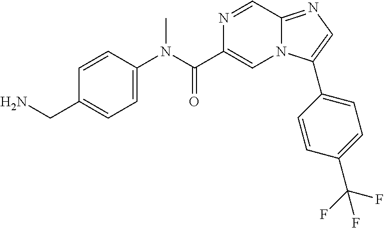 [SMILES: CN(C(=O)c1cn2c(-c3ccc(C(F)(F)F)cc3)cnc2cn1)c1ccc(CN)cc1]

The compound was synthesized using a procedure analogous to one use for synthesis of 212. M/Z=426.1 (M+1), r.t=1.15 mins.

### Synthesis of Compound 376

### N-(4-cyanophenyl)-2-methyl-3-[4-(trifluoromethyl)phenyl]imidazo[1,2-a]pyrazine-6-carboxamide

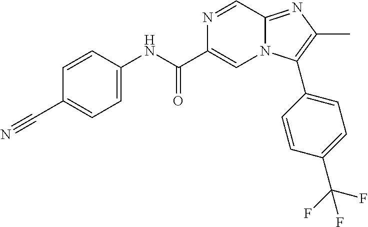 [SMILES: Cc1nc2cnc(C(=O)Nc3ccc(C#N)cc3)cn2c1-c1ccc(C(F)(F)F)cc1]

The compound was synthesized using a procedure analogous to one use for synthesis of 72 (method II) using intermediate 285-III. MS m/z 422.1 (M+H)+; r.t.=2.092.

### Synthesis of Compound 377

### N,3-bis(4-cyanophenyl)-N-methylimidazo[1,2-a]pyrazine-6-carboxamide

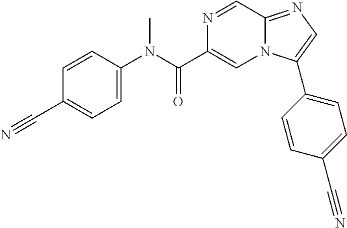 [SMILES: CN(C(=O)c1cn2c(-c3ccc(C#N)cc3)cnc2cn1)c1ccc(C#N)cc1]

The compound was synthesized using I-59 and an appropriate boronic acid/ester using a procedure similar Suzuki coupling protocol described in 228. MS m/z 379.2 (M+H)+; r.t.=1.910

### Synthesis of Compound 378

### N-(4-cyanophenyl)-N-methyl-3-(2-methylpyridin-4-yl)imidazo[1,2-a]pyrazine-6-carboxamide

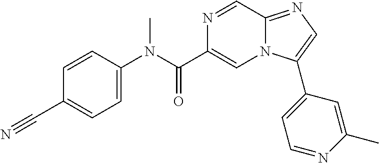 [SMILES: Cc1cc(-c2cnc3cnc(C(=O)N(C)c4ccc(C#N)cc4)cn23)ccn1]

The compound was synthesized using I-59 and an appropriate boronic acid/ester using a procedure similar Suzuki coupling protocol described in 228. M/Z=369.1 (M+1), r.t=0.94 mins.

### Synthesis of Compound 379

### N-(6-chloropyridin-3-yl)-N-methyl-3-(2-oxo-2,3-dihydro-1H-indol-5-yl)imidazo[1,2-a]pyrazine-6-carboxamide

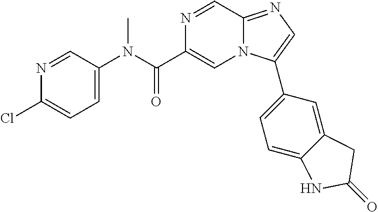 [SMILES: CN(C(=O)c1cn2c(-c3ccc4c(c3)CC(=O)N4)cnc2cn1)c1ccc(Cl)nc1]

The compound was synthesized using I-57 and an appropriate boronic acid/ester using a procedure similar Suzuki coupling protocol described in 228. M/Z=419.1 (M+1), r.t=1.11 mins.

### Synthesis of Compound 380

### 3-(2-aminopyrimidin-5-yl)-N-(4-cyanophenyl)-N-methylimidazo[1,2-a]pyrazine-6-carboxamide

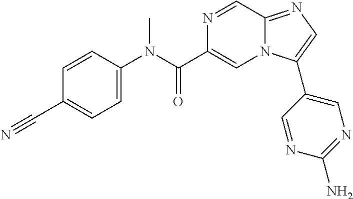 [SMILES: CN(C(=O)c1cn2c(-c3cnc(N)nc3)cnc2cn1)c1ccc(C#N)cc1]

The compound was synthesized using I-59 and an appropriate boronic acid/ester using a procedure similar Suzuki coupling protocol described in 228. M/Z=371.2.1 (M+1), r.t=0.99 mins.

### Synthesis of Compound 381

### N-(6-chloropyridin-3-yl)-3-ethenyl-N-methylimidazo[1,2-a]pyrazine-6-carboxamide

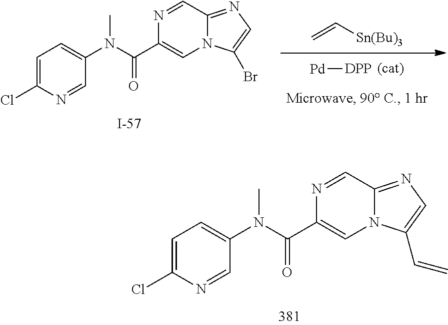

To a solution of 3-bromo-N-(4-chlorophenyl)-N-methylimidazo[1,2-a]pyrazine-6-carboxamide (40 mg, 0.109 mmols, 1 eq), in THF/water (4:1) was added tributyl vinyl tin (30 mg, 0.109 mmols, 1 eq) was added. The reaction mixture was heated at 90° C. for one hour. The desired compound N-(4-chlorophenyl)-N-methyl-3-vinylimidazo[1,2-a]pyrazine-6-carboxamide 5 mg (0.0159 mmol, 15%) was isolated via preparative HPLC. M/Z=313 (M+1), r.t=1.11 min.

### Synthesis of Compound 382

### N-(5-cyclobutylpyridin-2-yl)-N-methyl-3-[4-(trifluoromethyl)phenyl]imidazo[1,2-a]pyrazine-6-carboxamide

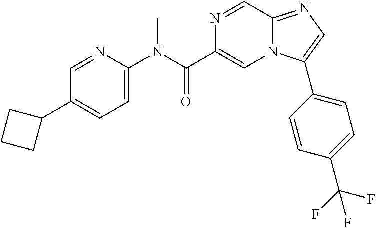 [SMILES: CN(C(=O)c1cn2c(-c3ccc(C(F)(F)F)cc3)cnc2cn1)c1ccc(C2CCC2)cn1]

The compound was synthesized using a procedure analogous to one use for synthesis of 72 (method II), using intermediate I-68. ¹H NMR (400 MHz, MeOD) δ 9.02 (s, 1H), 8.84-8.71 (m, 1H), 8.64-8.51 (m, 1H), 8.15 (s, 1H), 8.12-8.05 (m, 1H), 7.93 (d, J=3.0 Hz, 3H), 7.76 (s, 2H), 3.59 (s, 3H), 1.43 (d, J=15.0 Hz, 9H). ESI-MS m/z 454 [M+1]. RT: 1.49 min.

### Synthesis of Compound 383

### N-{[4-(morpholin-4-yl)phenyl]methyl}-3-[4-(trifluoromethyl)phenyl]imidazo[1,2-a]pyrazine-6-carboxamide

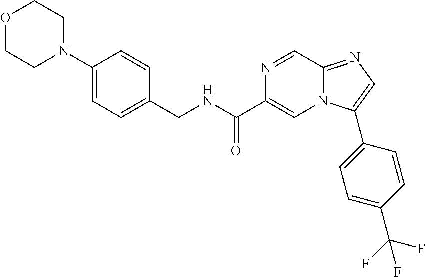 [SMILES: O=C(NCc1ccc(N2CCOCC2)cc1)c1cn2c(-c3ccc(C(F)(F)F)cc3)cnc2cn1]

The compound was synthesized using a procedure analogous to one use for synthesis of 72 (method II). ¹H NMR (300 MHz, DMSO-d₆) δ ppm 2.97-3.08 (m, 4H) 3.63-3.75 (m, 4H) 4.39 (d, J=6.45 Hz, 2H) 6.86 (m, J=8.79 Hz, 2H) 7.20 (m, J=8.79 Hz, 2H) 7.91-8.04 (m, 4H) 8.25 (s, 1H) 8.97 (d, J=1.47 Hz, 1H) 9.20 (d, J=1.17 Hz, 1H) 9.24 (t, J=6.30 Hz, 1H); MS (ESI) for C₂₅H₂₂F₃N₅O₂: 482 (M+H)⁺.

### Synthesis of Compound 384

### N-(6-tert-butylpyridin-3-yl)-N-methyl-3-[4-(trifluoromethyl)phenyl]imidazo[1,2-a]pyrazine-6-carboxamide

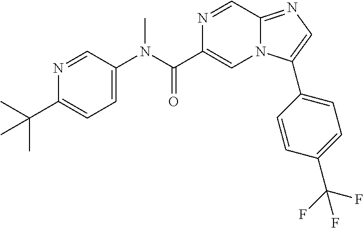 [SMILES: CN(C(=O)c1cn2c(-c3ccc(C(F)(F)F)cc3)cnc2cn1)c1ccc(C(C)(C)C)nc1]

The compound was synthesized using a procedure analogous to one use for synthesis of 72 (method II). M/Z 454.2 [M+1]. RT: 1.49 min.

### Synthesis of Compound 385

### -(4-cyanophenyl)-N-methyl-3-(pyridin-4-yl)imidazo[1,2-a]pyrazine-6-carboxamide

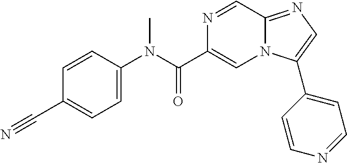 [SMILES: CN(C(=O)c1cn2c(-c3ccncc3)cnc2cn1)c1ccc(C#N)cc1]

The compound was synthesized using I-59 and an appropriate boronic acid/ester using a procedure similar Suzuki coupling protocol described in 228. M/Z=355.2 (M+1), r.t=0.74 mins.

### Synthesis of Compound 386

### N-(2-aminoethyl)-N-(4-cyanophenyl)-3-[4-(trifluoromethyl)phenyl]imidazo[1,2-a]pyrazine-6-carboxamide

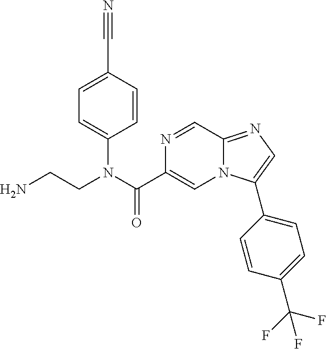 [SMILES: N#Cc1ccc(N(CCN)C(=O)c2cn3c(-c4ccc(C(F)(F)F)cc4)cnc3cn2)cc1]

386-1 was synthesized using a procedure analogous to one use for synthesis of 72 (method II) using I-33.

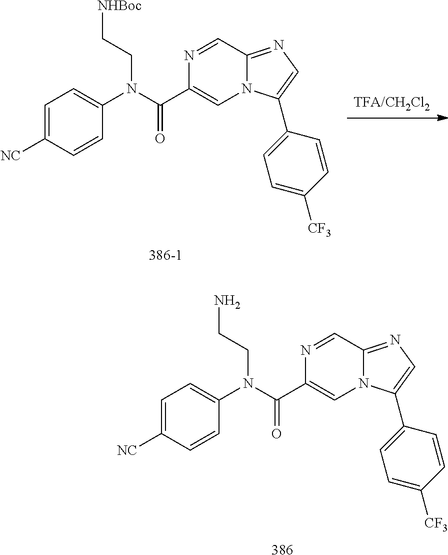 [SMILES: CC(C)(C)OC(=O)NCCN(C(=O)c1cn2c(-c3ccc(C(F)(F)F)cc3)cnc2cn1)c1ccc(C#N)cc1 ; "386-1" ; N#Cc1ccc(N(CCN)C(=O)c2cn3c(-c4ccc(C(F)(F)F)cc4)cnc3cn2)cc1 ; "386"]

TFA (0.20 mL) was added to a solution of tert-butyl(2-(N-(4-cyanophenyl)-3-(4-(trifluoromethyl)phenyl)imidazo[1,2-a]pyrazine-6-carboxamido)ethyl)carbamate (20 mg, 0.036 mmol) and dichloromethane (1.0 mL). The reaction stirred at room temperature for 2 hours. The solvent was removed and the crude product was purified by mass-trigger HPLC. LC/MS m/z 451.1 (M+H)+; r.t.=1.367.

### Synthesis of Compound 387

### N-(6-chloropyridin-3-yl)-3-(1H-indol-2-yl)-N-methylimidazo[1,2-a]pyrazine-6-carboxamide

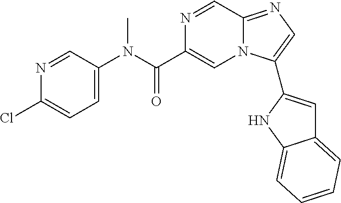 [SMILES: CN(C(=O)c1cn2c(-c3cc4ccccc4[nH]3)cnc2cn1)c1ccc(Cl)nc1]

The compound was synthesized using I-57 and an appropriate boronic acid/ester using a procedure similar Suzuki coupling protocol described in 228. M/Z=1.56 (M+1), r.t=403.1 mins.

### Synthesis of Compound 388

### N-(6-chloropyridin-3-yl)-3-{4-[(dimethylamino)methyl]phenyl}-N-methylimidazo[1,2-a]pyrazine-6-carboxamide

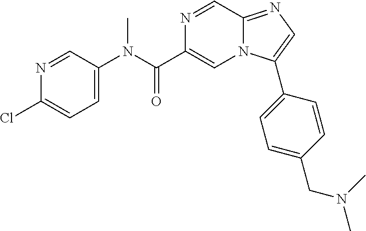 [SMILES: CN(C)Cc1ccc(-c2cnc3cnc(C(=O)N(C)c4ccc(Cl)nc4)cn23)cc1]

The compound was synthesized using I-57 and an appropriate boronic acid/ester using a procedure similar Suzuki coupling protocol described in 228. M/Z=421.2 (M+1), r.t=0.97 mins.

### Synthesis of Compound 389

### N-(4-cyano-1H-imidazol-5-yl)-3-[4-(trifluoromethyl)phenyl]imidazo[1,2-a]pyrazine-6-carboxamide

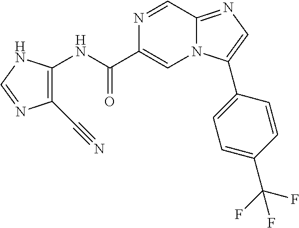 [SMILES: N#Cc1nc[nH]c1NC(=O)c1cn2c(-c3ccc(C(F)(F)F)cc3)cnc2cn1]

The compound was synthesized using a procedure analogous to one use for synthesis of 128. ¹H NMR (300 MHz, DMSO-d₆) δ ppm 9.32 (d, J=1.46 Hz, 1H) 9.12 (d, J=1.17 Hz, 1H) 8.34 (s, 1H) 7.97-8.11 (m, 4H) 7.80 (s, 1H); MS: 398 (M+H)⁺.

### Synthesis of Compound 390

### N-methyl-N-(3-methylpyridin-2-yl)-3-[4-(trifluoromethyl)phenyl]imidazo[1,2-a]pyrazine-6-carboxamide

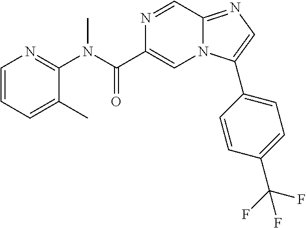 [SMILES: Cc1cccnc1N(C)C(=O)c1cn2c(-c3ccc(C(F)(F)F)cc3)cnc2cn1]

The compound was synthesized using a procedure analogous to one use for synthesis of 72 (method II). ¹H NMR (300 MHz, DMSO-d₆) ppm 9.32 (d, J=1.46 Hz, 1H) 9.12 (d, J=1.17 Hz, 1H) 8.34 (s, 1H) 7.97-8.11 (m, 4H) 7.80 (s, 1H); MS: 398 (M+H)⁺.

### Synthesis of Compound 391

### N-(4-cyanophenyl)-N-methyl-3-{1-methyl-1H-pyrazolo[3,4-b]pyridin-5-yl}imidazo[1,2-a]pyrazine-6-carboxamide

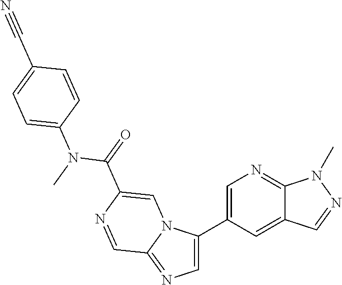 [SMILES: CN(C(=O)c1cn2c(-c3cnc4c(cnn4C)c3)cnc2cn1)c1ccc(C#N)cc1]

The compound was synthesized using I-59 and an appropriate boronic acid/ester using a procedure similar Suzuki coupling protocol described in 228. M/Z=409.1 (M+1), r.t=1.26 mins.

### Synthesis of Compound 392

### 4-({3-[4-(trifluoromethyl)phenyl]imidazo[1,2-a]pyrazin-6-yl}methyl)morpholine

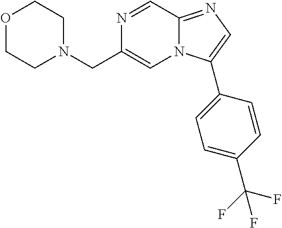 [SMILES: FC(F)(F)c1ccc(-c2cnc3cnc(CN4CCOCC4)cn23)cc1]

The compound was synthesized procedure analogous to the synthesis of I-15 and followed Suzuki coupling protocol described in 228. H-NMR (400 MHz, CDCL₃): 9.18 (d, J=1.31 Hz, 1H), 8.28 (s, 1H), 7.93 (s, 1H), 7.85 (d, J=7.91 Hz, 2H), 7.72 (d, J=7.91 Hz, 2H), 3.68-3.76 (m, 6H), 2.56-2.58 (m, 4H). LC-MS: 98.95%, 363.3 (M+H).

### Synthesis of Compound 393

### 5-{6-[(4-cyanophenyl)(methyl)carbamoyl]imidazo[1,2-a]pyrazin-3-yl}pyridine-2-carboxamide

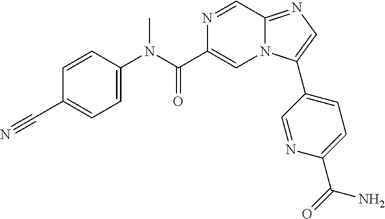 [SMILES: CN(C(=O)c1cn2c(-c3ccc(C(N)=O)nc3)cnc2cn1)c1ccc(C#N)cc1]

The compound was synthesized using I-59 and an appropriate boronic acid/ester using a procedure similar Suzuki coupling protocol described in 228. MS m/z 398.1 (M+H)+; r.t.=1.110.

### Synthesis of Compound 394

### N-[6-(dimethylamino)pyridin-3-yl]-N-methyl-3-[4-(trifluoromethyl)phenyl]imidazo[1,2-a]pyrazine-6-carboxamide

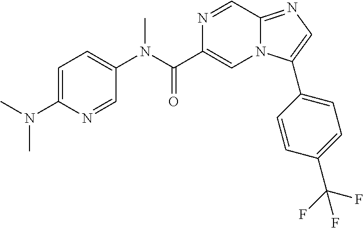 [SMILES: CN(C)c1ccc(N(C)C(=O)c2cn3c(-c4ccc(C(F)(F)F)cc4)cnc3cn2)cn1]

The compound was synthesized using a procedure analogous to one use for synthesis of 72 (method II). M/Z=441.2 (M+1), r.t=1.25 mins.

### Synthesis of Compound 395

### N-(6-methoxypyridazin-3-yl)-N-methyl-3-[4-(trifluoromethyl)phenyl]imidazo[1,2-a]pyrazine-6-carboxamide

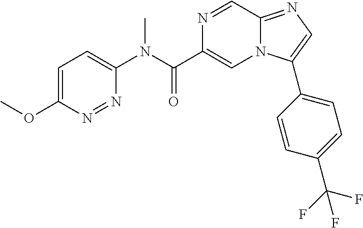 [SMILES: COc1ccc(N(C)C(=O)c2cn3c(-c4ccc(C(F)(F)F)cc4)cnc3cn2)nn1]

The compound was synthesized using a procedure analogous to one use for synthesis of 72 (method II). M/Z=429.1 (M+1), r.t=1.35 mins.

### Synthesis of Compound 396

### 3-(3-amino-1H-indazol-5-yl)-N-(4-cyanophenyl)-N-methylimidazo[1,2-a]pyrazine-6-carboxamide

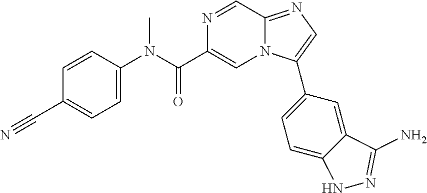 [SMILES: CN(C(=O)c1cn2c(-c3ccc4[nH]nc(N)c4c3)cnc2cn1)c1ccc(C#N)cc1]

The compound was synthesized using I-59 and an appropriate boronic acid/ester using a procedure similar Suzuki coupling protocol described in 228. MS m/z 409.2 (M+H)+; r.t.=1.081.

### Synthesis of Compound 397

### 1-({3-[4-(trifluoromethyl)phenyl]imidazo[1,2-a]pyrazin-6-yl}carbonyl)piperidine-3-carboxamide

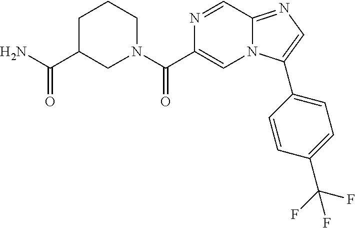 [SMILES: NC(=O)C1CCCN(C(=O)c2cn3c(-c4ccc(C(F)(F)F)cc4)cnc3cn2)C1]

The compound was synthesized using a procedure analogous to one use for synthesis of 72 (method II). ¹H NMR (300 MHz, DMSO-d₆) ppm 9.18 (d, J=1.47 Hz, 1H) 8.78 (d, J=1.47 Hz, 1H) 8.27 (s, 1H) 7.98 (q, J=8.69 Hz, 4H) 7.19-7.49 (m, 1H) 6.75-6.97 (m, 1H) 4.22-4.57 (m, 1H) 3.93 (d, J=11.72 Hz, 1H) 2.75-3.04 (m, 1H) 2.27-2.43 (m, 1H) 1.36-2.04 (m, 4H); MS (ESI) for C₂₀H₁₈F₃N₅O₂: 418 (M+H⁺).

### Synthesis of Compound 398

### N-(6-chloropyridin-3-yl)-N-methyl-3-[4-(methylamino)phenyl]imidazo[1,2-a]pyrazine-6-carboxamide

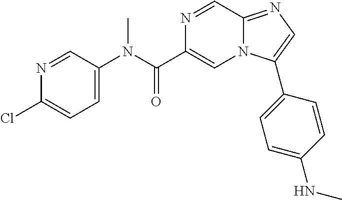 [SMILES: CNc1ccc(-c2cnc3cnc(C(=O)N(C)c4ccc(Cl)nc4)cn23)cc1]

The compound was synthesized using I-57 and an appropriate boronic acid/ester using a procedure similar Suzuki coupling protocol described in 228. M/Z=441.2 (M+1), r.t=1.25 mins.

### Synthesis of Compound 399

### N-(2-methoxypyrimidin-5-yl)-N-methyl-3-[4-(trifluoromethyl)phenyl]imidazo[1,2-a]pyrazine-6-carboxamide

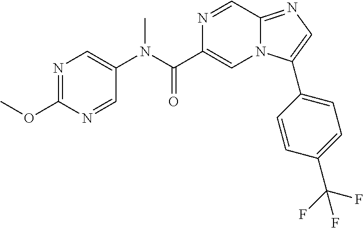 [SMILES: COc1ncc(N(C)C(=O)c2cn3c(-c4ccc(C(F)(F)F)cc4)cnc3cn2)cn1]

The compound was synthesized using a procedure analogous to one use for synthesis of 72 (method II). M/Z=429.1 (M+1), r.t=1.35 mins.

### Synthesis of Compound 400

### -(4-cyanophenyl)-N-methyl-3-(6-{[2-(morpholin-4-yl)ethyl]amino}pyridin-3-yl)imidazo[1,2-a]pyrazine-6-carboxamide

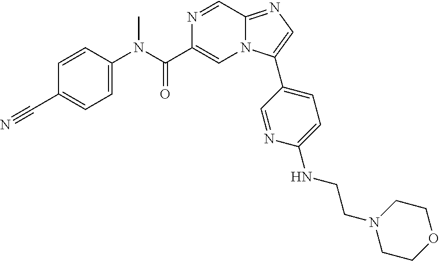 [SMILES: CN(C(=O)c1cn2c(-c3ccc(NCCN4CCOCC4)nc3)cnc2cn1)c1ccc(C#N)cc1]

The compound was synthesized using I-59 and an appropriate boronic acid/ester using a procedure similar Suzuki coupling protocol described in 228. M/Z=483.2 (M+1), r.t=0.93 mins.

### Synthesis of Compound 401

### N-(4-cyanophenyl)-3-(6-cyanopyridin-3-yl)-N-methylimidazo[1,2-a]pyrazine-6-carboxamide

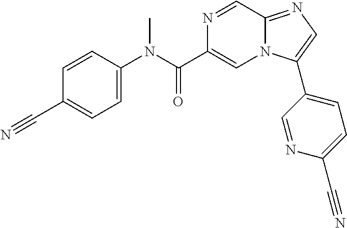 [SMILES: CN(C(=O)c1cn2c(-c3ccc(C#N)nc3)cnc2cn1)c1ccc(C#N)cc1]

The compound was synthesized using I-59 and an appropriate boronic acid/ester using a procedure similar Suzuki coupling protocol described in 228. M/Z=380.2 (M+1), r.t=1.28 mins.

### Synthesis of Compound 402

### 3-{4-[(1-amino-2-methylpropan-2-yl)oxy]phenyl}-N-(4-cyanophenyl)-N-methylimidazo[1,2-a]pyrazine-6-carboxamide

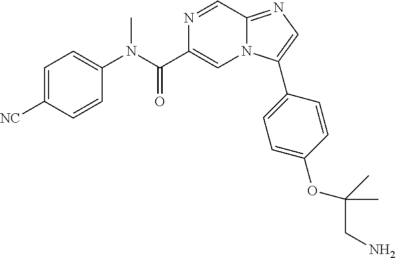 [SMILES: CN(C(=O)c1cn2c(-c3ccc(OC(C)(C)CN)cc3)cnc2cn1)c1ccc(C#N)cc1]

The compound was synthesized using a protocol described in 212. ¹H NMR (400 MHz, CDCl3) δ 8.81 (s, 1H), 8.74 (s, 1H), 8.23-8.06 (m, 1H), 7.84 (s, 1H), 7.53 (d, J=8.6 HZ, 2H), 7.42 (d, J=8.4 Hz, 2H), 7.19-7.12 (m, 3H), 5.23 (m, 2H), 3.49 (s, 3H), 2.11 (s, 2H), 1.41 (s, 6H). ESI-MS m/z 441 [M+1]. RT: 1.11 min.

### Synthesis of Compound 403

### 6-N-(6-chloropyridin-3-yl)-6-N-methyl-3-N-[4-(trifluoromethyl)phenyl]imidazo[1,2-a]pyrazine-3,6-dicarboxamide

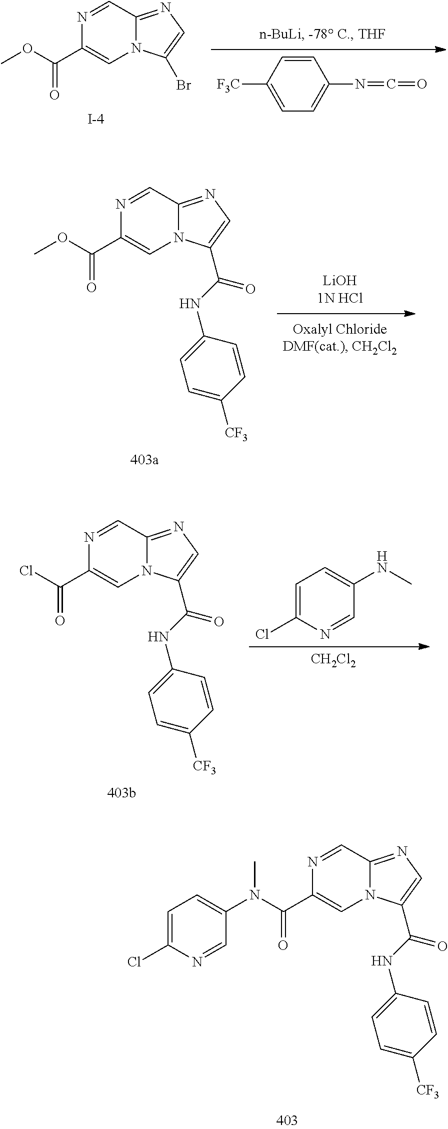

### Synthesis of methyl 3-((4-(trifluoromethyl)phenyl)carbamoyl)imidazo[1,2-a]pyrazine-6-carboxylate (403a)

To a solution of methyl 3-bromoimidazo[1,2-a]pyrazine-6-carboxylate I-4 (0.35 g, 1.36 mmol, 1 eq) in THF at −78° C. was added n-BuLi (1.70 mmol, 1.25 eq) drop by drop over ½ hr. The reaction mixture was stirred at −78° C. for 2 more hours at which point the isocyanate was added (pre-dissolved in THF) at this low temperature. The reaction mixture was stirred at −78° C. for 3 more hours and then was quenched at −78° C. with saturated ammonium chloride solution. The organics were extracted with ethyl acetate (3×30 mL), combined all the organics and dried over magnesium sulfate and isolated the desired compound via column chromatography. Yield=132 mg (0.362 mmol, 27%).

### Synthesis of 3-((4-(trifluoromethyl)phenyl)carbamoyl)imidazo[1,2-a]pyrazine-6-carbonyl chloride (403b)

To a solution of methyl 3-((4-(trifluoromethyl)phenyl)carbamoyl)imidazo[1,2-a]pyrazine-6-carboxylate (130 mg, 0.356 mmols, 1 eq) in THF/water (6:1) 6 mL was added LiOH (24 mg, 1.07 mmols, 3 eq). The reaction mixture was stirred at room temperature overnight. Removed all solvents, added the oxalyl chloride followed by catalytic DMF. Next day removed all solvents under vacuo and used for the next step.

### Synthesis of Compound 403

To a solution of 3-((4 (trifluoromethyl)phenyl)carbamoyl) imidazo[1,2-a]pyrazine-6-carbonyl chloride (19 mg, 0.051 mmol, 1 eq) dissolved in DCM was added the 6-chloro-N-methylpyridin-3-amine (18 mg, 0.128 mmols, 2.5 eq) followed by DIEA (16 mg, 0.128 mmol, 2.5 eq). The reaction mixture was stirred at room temperature for 6 hrs. The compound was purified using preparative HPLC and isolated as a TFA salt. M/Z=475.1 (M+1), r.t=1.72 mins.

### Synthesis of Compound 404

### 5-C-3-[4-(trifluoromethyl)phenyl]imidazo[1,2-a]pyrazine-6-1H-imidazole-4,5-dicarboxamide

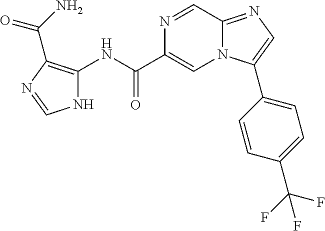 [SMILES: NC(=O)c1nc[nH]c1NC(=O)c1cn2c(-c3ccc(C(F)(F)F)cc3)cnc2cn1]

The compound was synthesized using a procedure analogous to one use for synthesis of 72 (method II). ¹H NMR (300 MHz, DMSO-d₆) ppm 7.44 (s, 1H), 7.93-8.14 (m, 4H), 8.34 (s, 1H), 9.13 (d, J=1.17 Hz, 1H), 9.33 (d, J=1.47 Hz, 1H). ESI-MS m/z 416 [M+H]⁺.

### Synthesis of Compound 405

### N-[1-(4-chlorophenyl)ethyl]-N-methyl-3-[4-(trifluoromethyl)phenyl]imidazo[1,2-a]pyrazine-6-carboxamide

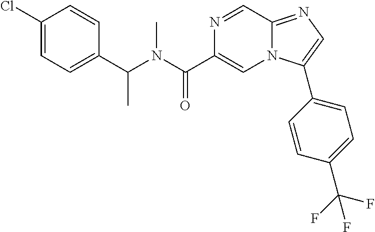 [SMILES: CC(c1ccc(Cl)cc1)N(C)C(=O)c1cn2c(-c3ccc(C(F)(F)F)cc3)cnc2cn1]

The compound was synthesized using a procedure analogous to one use for synthesis of 72 (method II). M/Z=459.1.2 (M+1), r.t=1.93 mins.

### Synthesis of Compound 406

### N-{4-[(1,3-thiazol-2-yl)sulfamoyl]phenyl}-3-[4-(trifluoromethyl)phenyl]imidazo[1,2-a]pyrazine-6-carboxamide

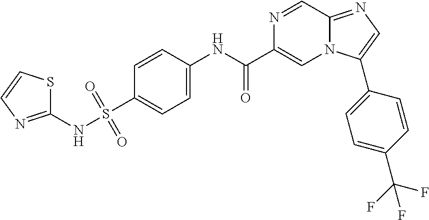 [SMILES: O=C(Nc1ccc(S(=O)(=O)Nc2nccs2)cc1)c1cn2c(-c3ccc(C(F)(F)F)cc3)cnc2cn1]

The compound was synthesized using a procedure analogous to one use for synthesis of 72 (method II). ¹H NMR (300 MHz, DMSO-d₆) ppm 6.77 (d, J=4.40 Hz, 1H), 7.20 (d, J=4.69 Hz, 1H), 7.72-7.85 (m, 1H), 7.97-8.12 (m, 6H), 8.33 (s, 1H), 9.13 (d, J=1.17 Hz, 1H), 9.31 (d, J=1.47 Hz, 1H), 10.93 (s, 1H). ESI-MS m/z 545 [M+H]⁺.

### Synthesis of Compound 407

### N-(6-chloropyridin-3-yl)-N-methyl-3-[6-(methylamino)pyridin-3-yl]imidazo[1,2-a]pyrazine-6-carboxamide

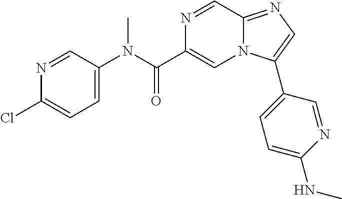 [SMILES: CNc1ccc(-c2cnc3cnc(C(=O)N(C)c4ccc(Cl)nc4)cn23)cn1]

The compound was synthesized using I-57 and an appropriate boronic acid/ester using a procedure similar Suzuki coupling protocol described in 228. ¹H NMR (400 MHz, MeOD) δ 9.10-9.04 (m, 1H), 8.91-8.87 (m, 1H), 8.50-8.41 (m, 2H), 8.27-8.13 (m, 3H), 7.50-7.40 (m, 2H), 3.54 (s, 3H), 3.18-3.04 (m, 3H). ESI-MS m/z 394 [M+H]+. RT: 0.90 min.

### Synthesis of Compound 408

### 3-[4-(2-amino-2-methylpropanamido)phenyl]-N-(4-cyanophenyl)-N-methylimidazo[1,2-a]pyrazine-6-carboxamide

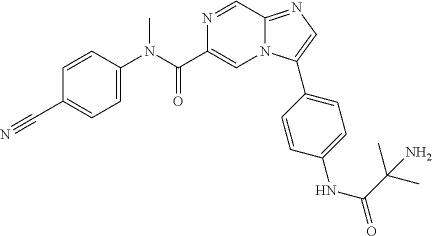 [SMILES: CN(C(=O)c1cn2c(-c3ccc(NC(=O)C(C)(C)N)cc3)cnc2cn1)c1ccc(C#N)cc1]

The compound was synthesized using a protocol described in 212. M/Z=454.1 (M+1), r.t=1.02 mins.

### Synthesis of Compound 409

### 3-[4-(2-aminoethoxyl)phenyl]-N-methyl-N-(5-methylpyridin-2-yl)imidazo[1,2-a]pyrazine-6-carboxamide

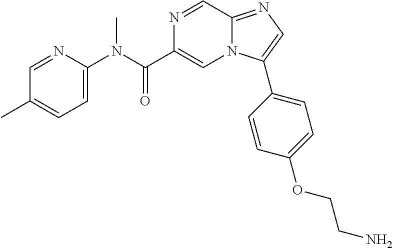 [SMILES: Cc1ccc(N(C)C(=O)c2cn3c(-c4ccc(OCCN)cc4)cnc3cn2)nc1]

The compound was synthesized using I-78 and an appropriate boronic acid/ester using a procedure similar Suzuki coupling protocol described in 228. ¹H NMR (400 MHz, MeOD) δ 8.13 (s, 1H), 7.68 (s, 2H), 7.44 (s, 3H), 7.31-7.25 (m, 1H), 7.16 (d, J=8.8 Hz, 3H), 5.00 (s, 2H), 4.09 (s, 2H), 3.26 (s, 3H), 2.26 (s, 3H). ESI-MS m/z 403 [M+H]+. RT: 1.12 min.

### Synthesis of Compound 410

### N-(6-chloropyridin-3-yl)-N-methyl-3-[4-(1,3,4-oxadiazol-2-yl)phenyl]imidazo[1,2-a]pyrazine-6-carboxamide

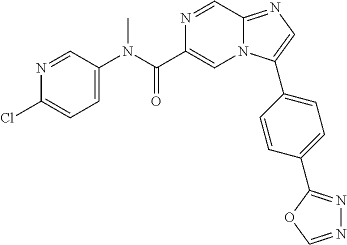 [SMILES: CN(C(=O)c1cn2c(-c3ccc(-c4nnco4)cc3)cnc2cn1)c1ccc(Cl)nc1]

The compound was synthesized using I-57 and an appropriate boronic acid/ester using a procedure similar Suzuki coupling protocol described in 228. ¹H NMR (400 MHz, MeOD) δ 9.09-8.97 (m, 1H), 8.85 (s, 1H), 8.80-8.72 (m, 1H), 8.70-8.61 (m, 1H), 8.32-8.23 (m, 1H), 8.17-8.06 (m, 2H), 7.94 (s, 2H), 7.82-7.76 (m, 1H), 7.50-7.40 (m, 1H), 3.55 (d, J=2.6 Hz, 3H). ESI-MS m/z 431 [M+H]+. RT: 1.25 min.

### Synthesis of Compound 411

### 6-fluoro-1-({8-methyl-3-[4-(trifluoromethyl)phenyl]imidazo[1,2-a]pyrazin-6-yl}carbonyl)-1,2,3,4-tetrahydroquinoline

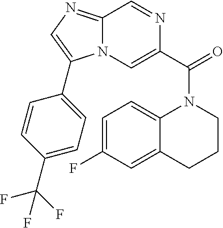 [SMILES: O=C(c1cn2c(-c3ccc(C(F)(F)F)cc3)cnc2cn1)N1CCCc2cc(F)ccc21]

¹H NMR (400 MHz, DMSO-d₆ at 80° C.): δ 8.95 (s, 1H), 7.75-7.85 (m, 5H), 7.23 (bs, 1H), 6.99 (m, 1H), 6.8 (bs, 1H), 3.67 (m, 2H), 2.81 (m, 2H), 2.15 (s, 3H), 1.94 (m, 2H). LC-MS: 98.42%; 455.10 (M+H).

### Synthesis of Compound 412

### N-(5-cyclopentylpyridin-2-yl)-N-methyl-3-[4-(trifluoromethyl)phenyl]imidazo[1,2-a]pyrazine-6-carboxamide

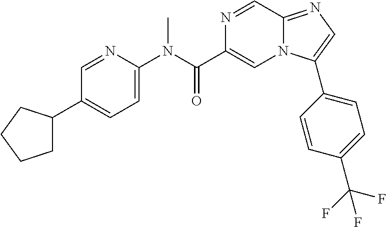 [SMILES: CN(C(=O)c1cn2c(-c3ccc(C(F)(F)F)cc3)cnc2cn1)c1ccc(C2CCCC2)cn1]

The compound was synthesized using a procedure analogous to one use for synthesis of 72 (method II). ¹H NMR (400 MHz, MeOD) δ 9.00-8.85 (m, 1H), 8.79-8.48 (m, 2H), 8.27-8.04 (m, 2H), 7.92 (d, J=5.1 Hz, 3H), 7.78-7.65 (m, 1H), 7.31-7.21 (m, 1H), 3.57 (s, 3H), 3.08-2.96 (m, 1H), 2.04 (s, 2H), 1.93-1.64 (m, 4H), 1.64-1.44 (m, 2H). ESI-MS m/z 466 [M+H]+. RT: 1.90 min.

### Synthesis of Compound 413

### N-(6-chloropyridin-3-yl)-N-methyl-3-[6-(trifluoromethyl)pyridin-3-yl]imidazo[1,2-a]pyrazine-6-carboxamide

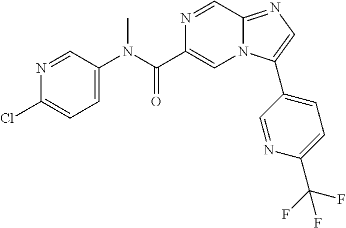 [SMILES: CN(C(=O)c1cn2c(-c3ccc(C(F)(F)F)nc3)cnc2cn1)c1ccc(Cl)nc1]

The compound was synthesized using I-57 and an appropriate boronic acid/ester using a procedure similar Suzuki coupling protocol described in 228. ¹H NMR (400 MHz, MeOD) δ 9.26-9.01 (m, 2H), 8.88-8.70 (m, 1H), 8.45 (d, J=8.1 Hz, 1H), 8.25 (d, J=11.4 Hz, 2H), 8.07 (d, J=8.2 Hz, 1H), 7.79 (s, 1H), 7.45 (d, J=8.5 Hz, 1H), 3.54 (s, 3H). ESI-MS m/z 433 [M+H]+. RT: 1.45 min.

### Synthesis of Compound 414

### N-[2-(4-fluorophenyl)propan-2-yl]-3-[4-(trifluoromethyl)phenyl]imidazo[1,2-a]pyrazine-6-carboxamide

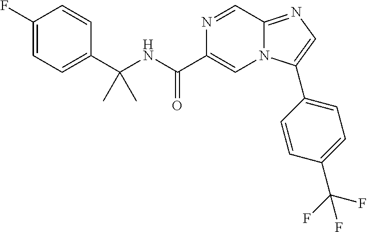 [SMILES: CC(C)(NC(=O)c1cn2c(-c3ccc(C(F)(F)F)cc3)cnc2cn1)c1ccc(F)cc1]

The compound was synthesized using a procedure analogous to one use for synthesis of 72 (method II). ¹H NMR (300 MHz, DMSO-d₅) ppm 9.25 (d, J=1.47 Hz, 1H) 8.90 (d, J=1.46 Hz, 1H) 8.60 (s, 1H) 8.28 (s, 1H) 7.91-8.05 (m, 4H) 7.40-7.50 (m, 2H) 7.03-7.16 (m, 2H) 1.74 (s, 6H); MS (ESI) for C₂₃H₁₈F₄N₄O: 443 (M+H⁺).

### Synthesis of Compound 415

### methyl N-(5-{6-[methyl(5-methylpyridin-2-yl)carbamoyl]imidazo[1,2-a]pyrazin-3-yl}pyridin-2-yl)carbamate

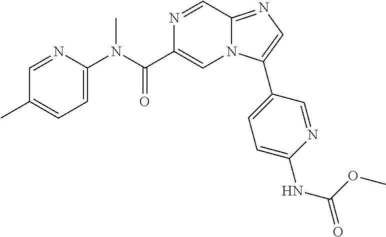

The compound was synthesized using I-78 and an appropriate boronic acid/ester using a procedure similar Suzuki coupling protocol described in 228. M/Z=418.2 (M+1), r.t=1.01 mins.

### Synthesis of Compound 416

### N-methyl-N-{[1,2,4]triazolo[4,3-a]pyridin-5-yl}-3-[4-(trifluoromethyl)phenyl]imidazo[1,2-a]pyrazine-6-carboxamide

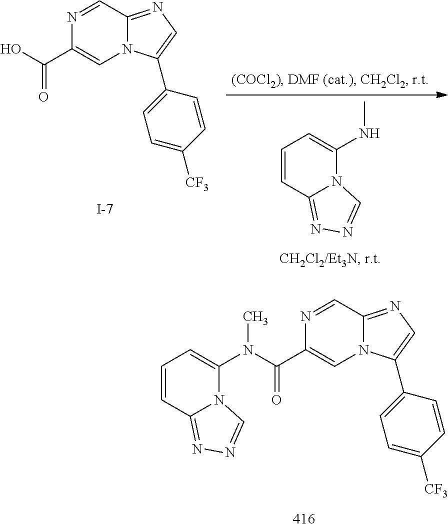

Oxalyl chloride (0.123 mg, 0.97 mmol) was added drop wise to a solution of 3-(4-(trifluoromethyl)phenyl)imidazo[1,2-a]pyrazine-6-carboxylic acid (30 mg, 0.097 mmol), DMF (1 drop) and dry dichloromethane (3 mL) at room temperature (gas evolution). After 30 minutes at room temperature, the solvent was removed in vacuo. The crude acid chloride was dissolved in dry dichloromethane (5 mL) and a solution of N-methyl-[1,2,4]triazolo[4,3-a]pyridin-5-amine I-28 (41 mg, 0.279 mmol in 2.0 mL of dichloromethane) was added drop wise at room temperature. Triethylamine (28 mg, 0.279 mmol) was added and the reaction stirred at room temperature for 3 hours. The solvent was removed in vacuo and the crude material was purified by reverse phase HPLC to yield the desired product. The product was characterized by reverse phase HPLC using method B. (ES, m/z): [M+H⁺] 438.1. Retention time=1.43 mins.

### Synthesis of Compound 417

### 4-tert-butyl-N-methyl-N-{3-[4-(trifluoromethyl)phenyl]imidazo[1,2-a]pyrazin-6-yl}benzamide

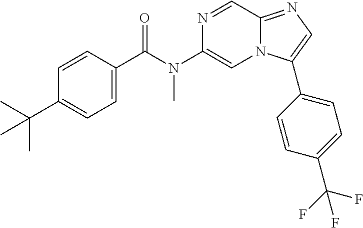 [SMILES: CN(C(=O)c1ccc(C(C)(C)C)cc1)c1cn2c(-c3ccc(C(F)(F)F)cc3)cnc2cn1]

1H NMR (400 MHz, CDCl₃) δ 9.11 (d, J=1.3, 1H), 7.88 (s, 1H), 7.71 (s, 1H), 7.63 (d, J=8.2, 2H), 7.41-7.30 (m, 4H), 7.01 (d, J=7.9, 2H), 3.58 (s, 3H), 1.24 (s, 9H). HRMS calcd for C₂₅H₂₄F₃N₄O [M+H]+453.18; found 453.2.

### Synthesis of Compound 418

### N-(6-chloropyridin-3-yl)-3-[3-(dimethylamino)phenyl]-N-methylimidazo[1,2-a]pyrazine-6-carboxamide

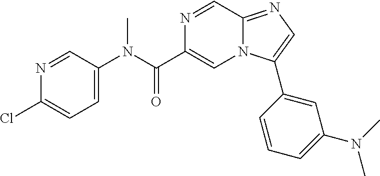 [SMILES: CN(C)c1cccc(-c2cnc3cnc(C(=O)N(C)c4ccc(Cl)nc4)cn23)c1]

The compound was synthesized using I-57 and an appropriate boronic acid/ester using a procedure similar Suzuki coupling protocol described in 228. ¹H NMR (400 MHz, MeOD) δ 8.99 (s, 1H), 8.88-8.62 (m, 2H), 8.25 (s, 1H), 8.05 (s, 1H), 7.77 (s, 1H), 7.56 (s, 1H), 7.44 (d, J=8.5 Hz, 1H), 7.23 (d, J=22.3 Hz, 2H), 3.54 (s, 6H), 3.15 (s, 3H). ESI-MS m/z 407 [M+H]+. RT: 1.10 min.

### Synthesis of Compound 419

### N-(6-chloropyridin-3-yl)-3-(4-methanesulfonamidophenyl)-N-methylimidazo[1,2-a]pyrazine-6-carboxamide

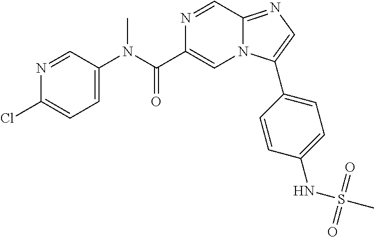 [SMILES: CN(C(=O)c1cn2c(-c3ccc(NS(C)(=O)=O)cc3)cnc2cn1)c1ccc(Cl)nc1]

The compound was synthesized using I-57 and an appropriate boronic acid/ester using a procedure similar Suzuki coupling protocol described in 228. M/Z=457.1 (M+1), r.t=1.23 mins.

### Synthesis of Compound 420

### N-methyl-N-(2-methylpyridin-3-yl)-3-[4-(trifluoromethyl)phenyl]imidazo[1,2-a]pyrazine-6-carboxamide

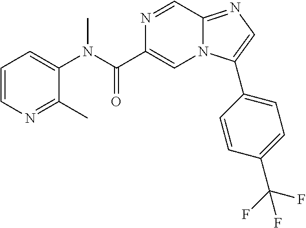 [SMILES: Cc1ncccc1N(C)C(=O)c1cn2c(-c3ccc(C(F)(F)F)cc3)cnc2cn1]

M/Z=412.2 (M+1), r.t=1.37 mins.

### Synthesis of Compound 421

### N-methyl-N-(5-methylpyrimidin-2-yl)-3-[4-(trifluoromethyl)phenyl]imidazo[1,2-a]pyrazine-6-carboxamide

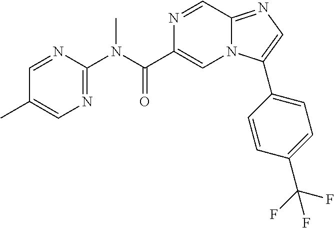 [SMILES: Cc1cnc(N(C)C(=O)c2cn3c(-c4ccc(C(F)(F)F)cc4)cnc3cn2)nc1]

The compound was synthesized using a procedure analogous to one use for synthesis of 72 (method II). M/Z=435 (M+1), r.t=1.76 mins.

### Synthesis of Compound 422

### N-(6-chloropyridin-3-yl)-3-(4-fluorophenyl)-N-methylimidazo[1,2-a]pyrazine-6-carboxamide

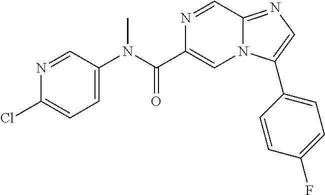 [SMILES: CN(C(=O)c1cn2c(-c3ccc(F)cc3)cnc2cn1)c1ccc(Cl)nc1]

The compound was synthesized using I-57 and an appropriate boronic acid/ester using a procedure similar Suzuki coupling protocol described in 228. M/Z=382.0 (M+1), r.t=1.43 mins.

### Synthesis of Compound 423

### N-methyl-N-(4-phenyl-1,3-thiazol-2-yl)-3-[4-(trifluoromethyl)phenyl]imidazo[1,2-a]pyrazine-6-carboxamide

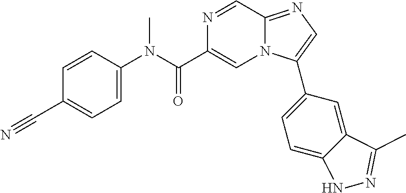 [SMILES: Cc1n[nH]c2ccc(-c3cnc4cnc(C(=O)N(C)c5ccc(C#N)cc5)cn34)cc12]

The compound was synthesized using I-59 and an appropriate boronic acid/ester using a procedure similar Suzuki coupling protocol described in 228. ¹H NMR (400 MHz, CDCl₃): δ 9.2 (s, 1H), 8.99 (s, 1H), 8.06 (s, 1H), 7.94 (d, J=7.47 Hz, 2H), 7.76-7.84 (m, 4H), 7.43 (m, 2H), 7.26-7.29 (m, 2H), 4.06 (s, 3H). LC-MS: 99.2%; 480.03 (M+H).

### Synthesis of Compound 424

### cyanophenyl)-N-methyl-3-(3-methyl-1H-indazol-5-yl)imidazo[1,2-a]pyrazine-6-carboxamide

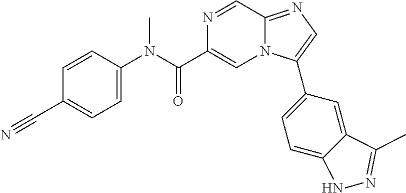 [SMILES: Cc1n[nH]c2ccc(-c3cnc4cnc(C(=O)N(C)c5ccc(C#N)cc5)cn34)cc12]

The compound was synthesized using I-59 and I-29 using a procedure similar Suzuki coupling protocol described in 228. M/Z=408.1 (M+1), r.t=1.26 mins.

### Synthesis of Compound 425

### N-(4-acetylphenyl)-N-methyl-3-[4-(trifluoromethyl)phenyl]imidazo[1,2-a]pyrazine-6-carboxamide

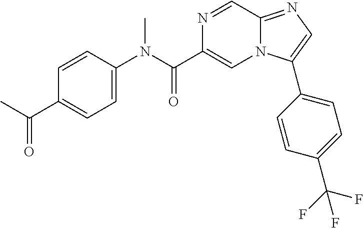 [SMILES: CC(=O)c1ccc(N(C)C(=O)c2cn3c(-c4ccc(C(F)(F)F)cc4)cnc3cn2)cc1]

The compound was synthesized using a procedure analogous to one use for synthesis of 72 (method II). M/Z=439.1 (M+1), r.t=1.61 mins.

### Synthesis of Compound 426

### 1-({3-[4-(trifluoromethyl)phenyl]imidazo[1,2-a]pyrazin-6-yl}methyl)piperidin-2-one

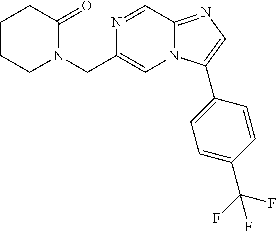 [SMILES: O=C1CCCCN1Cc1cn2c(-c3ccc(C(F)(F)F)cc3)cnc2cn1]

The compound was synthesized using a protocol described for the synthesis of I-15. ¹H NMR (400 MHz, CD₃OD): δ 9.06 (s, 1H), 8.57 (s, 1H), 8.07 (s, 1H), 7.89-7.94 (m, 4H), 4.67 (s, 2H), 3.55 (t, J=5.71 Hz, 2H), 2.37 (t, J=5.71 Hz, 2H), 1.81-1.82 (m, 4H). LC-MS: 97.99%; 375.23 (M+H).

### Synthesis of Compound 427

### N-(4-cyanophenyl)-3-[4-(dimethylcarbamoyl)phenyl]-N-methylimidazo[1,2-a]pyrazine-6-carboxamide

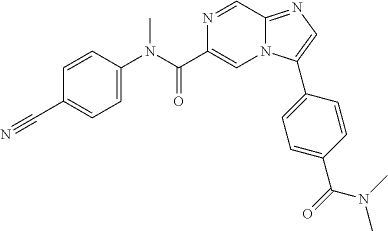 [SMILES: CN(C)C(=O)c1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. M/Z=425.1 (M+1), r.t=1.33 mins.

### Synthesis of Compound 428

### N-(4-cyanophenyl)-3-(1H-indol-5-yl)-N-methylimidazo[1,2-a]pyrazine-6-carboxamide

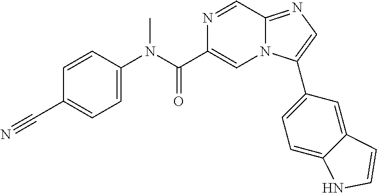 [SMILES: CN(C(=O)c1cn2c(-c3ccc4[nH]ccc4c3)cnc2cn1)c1ccc(C#N)cc1]

The compound was synthesized using I-59 and an appropriate boronic acid/ester using a procedure similar Suzuki coupling protocol described in 228. M/Z=393.2 (M+1), r.t=1.76 mins.

### Synthesis of Compound 429

### N-(6-chloropyridin-3-yl)-3-[4-(difluoromethoxy)phenyl]-N-methylimidazo[1,2-a]pyrazine-6-carboxamide

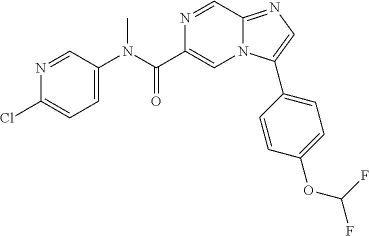 [SMILES: CN(C(=O)c1cn2c(-c3ccc(OC(F)F)cc3)cnc2cn1)c1ccc(Cl)nc1]

The compound was synthesized using I-57 and an appropriate boronic acid/ester using a procedure similar Suzuki coupling protocol described in 228. MS m/z 430.1 (M+H)+; r.t.=1.568.

### Synthesis of Compound 430

### N-(4-fluoro-2-methoxyphenyl)-N-methyl-3-[4(trifluoromethyl)phenyl]imidazo[1,2-a]pyrazine

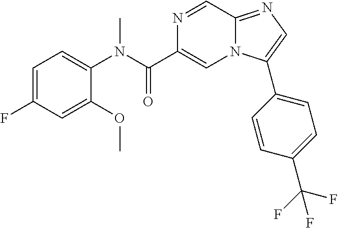 [SMILES: COc1cc(F)ccc1N(C)C(=O)c1cn2c(-c3ccc(C(F)(F)F)cc3)cnc2cn1]

The compound was synthesized using a procedure analogous to one use for synthesis of 72 (method II). MS m/z 445.1 (M+H)+; r.t.=1.736.

### Synthesis of Compound 431

### N-(3,4-difluorophenyl)-3-(4-methoxyphenyl)-N-methylimidazo[1,2-a]pyrazine-6-carboxamide

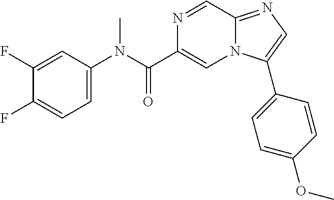 [SMILES: COc1ccc(-c2cnc3cnc(C(=O)N(C)c4ccc(F)c(F)c4)cn23)cc1]

The compound was synthesized using I-65 and an appropriate boronic acid/ester using a procedure similar Suzuki coupling protocol described in 228. MS m/z 395.2 (M+H)+; r.t.=1.990.

### Synthesis of Compound 432

### N-(4-chlorophenyl)-N-methyl-3-(4-methylphenyl)imidazo[1,2-a]pyrazine-6-carboxamide

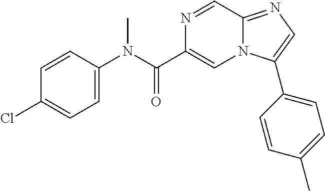 [SMILES: Cc1ccc(-c2cnc3cnc(C(=O)N(C)c4ccc(Cl)cc4)cn23)cc1]

The compound was synthesized using I-11 and an appropriate boronic acid/ester using a procedure similar Suzuki coupling protocol described in 228. MS m/z 377.2 (M+H)+; r.t.=2.140.

### Synthesis of Compound 433

### 3-[4-(difluoromethoxy)phenyl]-N-(4-fluorophenyl)-N-methylimidazo[1,2-a]pyrazine-6-carboxamide

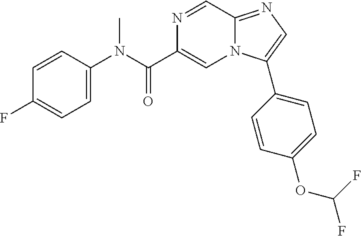 [SMILES: CN(C(=O)c1cn2c(-c3ccc(OC(F)F)cc3)cnc2cn1)c1ccc(F)cc1]

The compound was synthesized using I-64 and an appropriate boronic acid/ester using a procedure similar Suzuki coupling protocol described in 228. ¹H NMR (400 MHz, DMSO) δ 8.82 (s, 1H), 8.72 (s, 1H), 8.09 (s, 1H), 7.75 (d, J=8.7, 2H), 7.42 (d, J=8.6, 2H), 7.39 (t, J=76, 1H), 7.31 (s, 2H), 7.12 (t, J=8.6, 2H), 3.40 (s, 3H); MS m/z 413.1 (M+H)+; r.t.=1.050.

### Synthesis of Compound 434

### N-(5-bromopyrimidin-2-yl)-N-methyl-3-[4-(trifluoromethyl)phenyl]imidazo[1,2-a]pyrazine-6-carboxamide

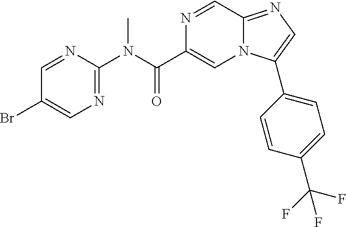 [SMILES: CN(C(=O)c1cn2c(-c3ccc(C(F)(F)F)cc3)cnc2cn1)c1ncc(Br)cn1]

The compound was synthesized using a procedure analogous to one use for synthesis of 72 (method II). MS m/z 477.0 (M+H)+; r.t.=2.310.

### Synthesis of Compound 435

### N-(5-chloropyridin-2-yl)-N-methyl-3-[4-(trifluoromethyl)phenyl]imidazo[1,2-a]pyrazine-6-carboxamide

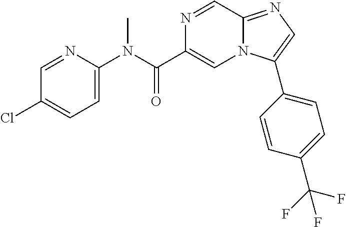 [SMILES: CN(C(=O)c1cn2c(-c3ccc(C(F)(F)F)cc3)cnc2cn1)c1ccc(Cl)cn1]

The compound was synthesized using a procedure analogous to one use for synthesis of 72 (method II) using I-36. MS m/z 432.1 (M+H)+; r.t.=2.200.

### Synthesis of Compound 436

### N-methyl-N-[4-(propan-2-yloxy)phenyl]-3-[4-(trifluoromethyl)phenyl]imidazo[1,2-a]pyrazine-6-carboxamide

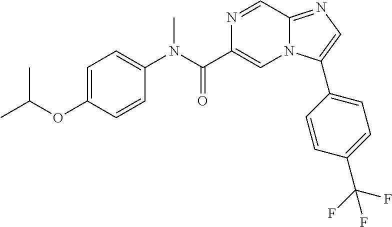 [SMILES: CC(C)Oc1ccc(N(C)C(=O)c2cn3c(-c4ccc(C(F)(F)F)cc4)cnc3cn2)cc1]

The compound was synthesized using a procedure analogous to one use for synthesis of 72 (method II). MS m/z 455.1 (M+H)+; r.t.=2.270.

### Synthesis of Compound 437

### 4-[({3-[4-(dimethylamino)phenyl]imidazo[1,2-a]pyrazin-6-yl}methyl)(methyl)amino]benzonitrile

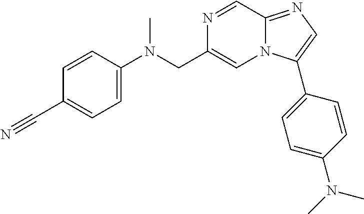 [SMILES: CN(C)c1ccc(-c2cnc3cnc(CN(C)c4ccc(C#N)cc4)cn23)cc1]

The compound was synthesized using I-15 and an appropriate boronic acid/ester using a procedure similar Suzuki coupling protocol described in 228. MS m/z 383.1 (M+H)+; r.t.=1.447.

### Synthesis of Compound 438

### N-(4-cyanophenyl)-3-[3-(dimethylamino)phenyl]-N-methylimidazo[1,2-a]pyrazine-6-carboxamide

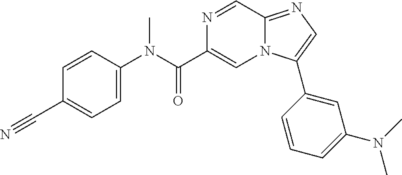 [SMILES: CN(C)c1cccc(-c2cnc3cnc(C(=O)N(C)c4ccc(C#N)cc4)cn23)c1]

The compound was synthesized using I-59 and an appropriate boronic acid/ester using a procedure similar Suzuki coupling protocol described in 228. M/Z=397.1 (M+1), r.t=1.27 mins.

### Synthesis of Compound 439

### 3-(6-chloropyridin-3-yl)-N-(4-cyanophenyl)-N-methylimidazo[1,2-a]pyrazine-6-carboxamide

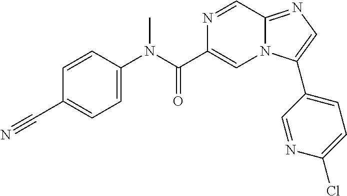 [SMILES: CN(C(=O)c1cn2c(-c3ccc(Cl)nc3)cnc2cn1)c1ccc(C#N)cc1]

The compound was synthesized using I-59 and an appropriate boronic acid/ester using a procedure similar Suzuki coupling protocol described in 228. M/Z=389.1 (M+1), r.t=1.73 mins.

### Synthesis of Compound 440

### N-(3-methoxyphenyl)-N-methyl-3-[4-(trifluoromethyl)phenyl]imidazo[1,2-a]pyrazine-6-carboxamide

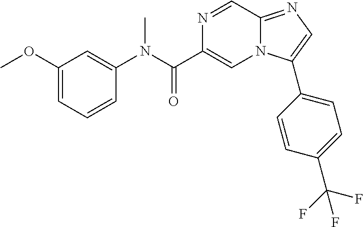 [SMILES: COc1cccc(N(C)C(=O)c2cn3c(-c4ccc(C(F)(F)F)cc4)cnc3cn2)c1]

The compound was synthesized using a procedure analogous to one use for synthesis of 72 (method II). MS m/z 427.1 (M+H)+; r.t.=1.692.

### Synthesis of Compound 441

### N-(4-cyanophenyl)-3-(4-methoxy-2-methylphenyl)-N-methylimidazo[1,2-a]pyrazine-6-carboxamide

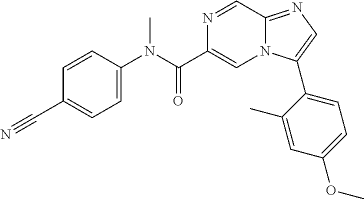 [SMILES: COc1ccc(-c2cnc3cnc(C(=O)N(C)c4ccc(C#N)cc4)cn23)c(C)c1]

The compound was synthesized using I-59 and an appropriate boronic acid/ester using a procedure similar Suzuki coupling protocol described in 228. M/Z=398.2 (M+1), r.t=1.94 mins.

### Synthesis of Compound 442

### N-(7-chloro-1,3-benzothiazol-2-yl)-3-[4-(trifluoromethyl)phenyl]imidazo[1,2-a]pyrazine-6-carboxamide

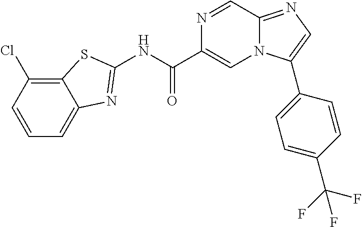 [SMILES: O=C(Nc1nc2cccc(Cl)c2s1)c1cn2c(-c3ccc(C(F)(F)F)cc3)cnc2cn1]

The compound was synthesized using I-72 and an appropriate boronic acid/ester using a procedure similar Suzuki coupling protocol described in 228. ¹H NMR (300 MHz, DMSO-d₆) ppm 7.41 (d, J=9.38 Hz, 1H), 7.69 (d, J=8.50 Hz, 1H), 8.05 (q, J=8.40 Hz, 5H), 8.31 (s, 1H), 9.29 (s, 3H). ESI-MS m/z 474 [M+H]⁺.

### Synthesis of Compound 443

### 4-fluoro-N-methyl-N-(3-{4-[5-(methylamino)-1,3,4-thiadiazol-2-yl]phenyl}imidazo[1,2-a]pyrazin-6-yl)benzamide

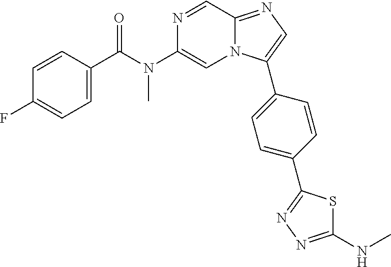 [SMILES: CNc1nnc(-c2ccc(-c3cnc4cnc(N(C)C(=O)c5ccc(F)cc5)cn34)cc2)s1]

1H NMR (400 MHz, MeOD) δ 8.95 (s, 1H), 8.14 (s, 1H), 7.92 (s, 1H), 7.82 (d, J=8.3, 2H), 7.35 (dd, J=5.3, 8.7, 2H), 7.26 (d, J=8.3, 2H), 6.99 (t, J=8.7, 2H), 3.46 (s, 3H), 3.03 (s, 3H). HRMS calcd for C₂₃H₁₇FN₇OS [M+H]+460.13; found 460.1.

### Synthesis of Compound 444

### 3-{6-[(6-fluoro-1,2,3,4-tetrahydroquinolin-1-yl)carbonyl]imidazo[1,2-a]pyrazin-3-yl}-N,N-dimethylaniline

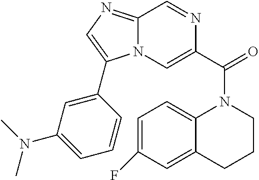 [SMILES: CN(C)c1cccc(-c2cnc3cnc(C(=O)N4CCCc5cc(F)ccc54)cn23)c1]

The compound was synthesized using I-72 and an appropriate boronic acid/ester using a procedure similar Suzuki coupling protocol described in 228. LCMS: Rₜ=1.20 min, ES⁺-MS m/z 416 [M+H]⁺.

### Synthesis of Compound 445

### N,3-bis[4-(trifluoromethyl)phenyl]imidazo[1,2-a]pyrazine-6-carboxamide

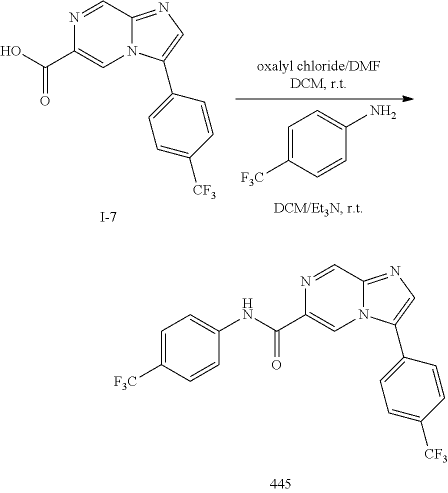

N,3-bis(4-(trifluoromethyl)phenyl)imidazo[1,2-a]pyrazine-6-carboxamide was prepared according to the procedure described for the synthesis of N-(4-cyanophenyl)-N-methyl-3-(4-(trifluoromethyl)phenyl)imidazo[1,2-a]pyrazine-6-carboxamide by substituting 4-trifluoromethylaniline for 4-cyano-N-methyl aniline. LC/MS m/z 451.1 (M+H)+; r.t.=2.650

### Synthesis of Compound 446

### N-(4-cyanophenyl)-N-methyl-3-{4-[5-(methylamino)-1,3,4-oxadiazol-2-yl]phenyl}imidazo[1,2-a]pyrazine-6-carboxamide

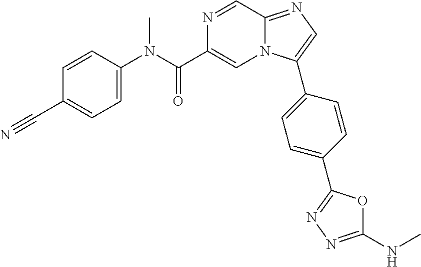 [SMILES: CNc1nnc(-c2ccc(-c3cnc4cnc(C(=O)N(C)c5ccc(C#N)cc5)cn34)cc2)o1]

To a solution of 3-bromo-N-(4-cyanophenyl)-N-methylimidazo[1,2-a]pyrazine-6-carboxamide (125 mg, 0.351 mmols, 1 eq) I-59 in THF/water (4:1) 12 mL was added N-methyl-5-(4-(4,4,5,5-tetramethyl-1,3,2-dioxaborolan-2-yl)phenyl)-1,3,4-oxadiazol-2-amine I-60 (158 mg, 0.526 mmol, 1.5 eq) followed by Pd-DPP (10 mol %) and K₂HPO₄ (152 mg, 0.88 mmol, 2.5 eq). The reaction mixture was heated at 150° C. for 1 hr. The reaction was quenched with sodium carbonate solution and the reaction mixture was extracted with ethyl acetate (3×15 mL). The organics were combined and dried over sodium sulfate and were using flash column chromatography. The reaction yielded 12 mg of the desired compound. ¹H NMR (400 MHz, MeOD) δ 8.98-8.87 (m, 1H), 8.78-8.70 (m, 1H), 8.11 (s, 2H), 7.91-7.74 (m, 2H), 7.67 (s, 2H), 7.62-7.55 (m, 1H), 7.47-7.38 (m, 2H), 3.56 (s, 3H), 2.67 (s, 3H). ESI-MS m/z 451 [M+H]+. RT: 1.50 min.

### Synthesis of Compound 447

### 4-{6-[(7-fluoro-3,3-dimethyl-3,4-dihydro-2H-1,4-benzoxazin-4-yl)carbonyl]imidazo[1,2-a]pyrazin-3-yl}benzamide

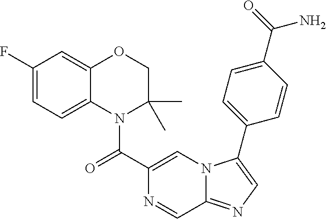 [SMILES: CC1(C)COc2cc(F)ccc2N1C(=O)c1cn2c(-c3ccc(C(N)=O)cc3)cnc2cn1]

The compound was synthesized using I-71 and an appropriate boronic acid/ester using a procedure similar Suzuki coupling protocol described in 228. ¹H NMR (400 MHz, CDCl₃): δ 8.88 (s, 2H), 7.99-8.02 (m, 3H), 7.66 (d, J=7.9 Hz, 2H), 6.67-6.7 (m, 1H), 6.27-6.36 (m, 2H), 4.09 (s, 2H), 1.59 (s, 6H). LC-MS: 97.98%; 446 (M+H).

### Synthesis of Compound 448

### 4-{6-[(6-fluoro-1,2,3,4-tetrahydroquinolin-1-yl)carbonyl]imidazo[1,2-a]pyrazin-3-yl}benzamide

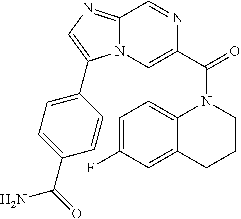 [SMILES: NC(=O)c1ccc(-c2cnc3cnc(C(=O)N4CCCc5cc(F)ccc54)cn23)cc1]

The compound was synthesized using I-72 and an appropriate boronic acid/ester using a procedure similar Suzuki coupling protocol described in 228. LCMS: Rₜ=1.00 min, ES⁺-MS m/z 416 [M+H]⁺.

### Synthesis of Compound 449

### N-(4-fluorophenyl)-N-methyl-3-[4-(1,3,4-thiadiazol-2-yl)phenyl]imidazo[1,2-a]pyrazine-6-carboxamide

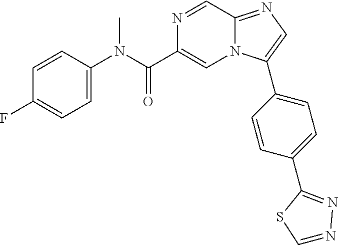 [SMILES: CN(C(=O)c1cn2c(-c3ccc(-c4nncs4)cc3)cnc2cn1)c1ccc(F)cc1]

The compound was synthesized using I-64 and an appropriate boronic acid/ester using a procedure similar Suzuki coupling protocol described in 228. ¹H NMR (400 MHz, MeOD) δ 9.59-9.50 (m, 1H), 8.93-8.71 (m, 2H), 8.32-8.20 (m, 2H), 8.16-8.06 (m, 1H), 7.87-7.73 (m, 2H), 7.38-7.22 (m, 2H), 7.15-6.98 (m, 2H), 3.51 (s, 3H). ESI-MS m/z 431 [M+1]. RT: 1.65 min.

### Synthesis of Compound 450

### 4-{6-[(6-fluoro-1,2,3,4-tetrahydroquinolin-1-yl)carbonyl]imidazo[1,2-a]pyrazin-3-yl}-N-methylbenzamide

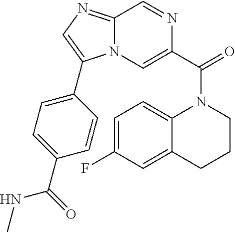 [SMILES: CNC(=O)c1ccc(-c2cnc3cnc(C(=O)N4CCCc5cc(F)ccc54)cn23)cc1]

The compound was synthesized using I-72 and an appropriate boronic acid/ester using a procedure similar Suzuki coupling protocol described in 228. LCMS: Rₜ=1.06 min, ES⁺-MS m/z 430 [M+H]⁺.

### Synthesis of Compound 451

### N-(4-{6-[(7-fluoro-3,3-dimethyl-3,4-dihydro-2H-1,4-benzoxazin-4-yl)carbonyl]imidazo[1,2-a]pyrazin-3-yl}phenyl)acetamide

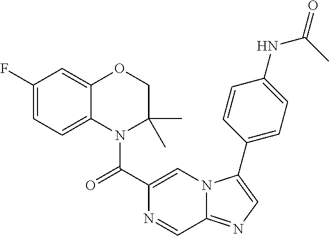 [SMILES: CC(=O)Nc1ccc(-c2cnc3cnc(C(=O)N4c5ccc(F)cc5OCC4(C)C)cn23)cc1]

The compound was synthesized using I-71 and an appropriate boronic acid/ester using a procedure similar Suzuki coupling protocol described in 228. ¹H NMR (400 MHz, in CDCl₃): δ 8.84 (d, J=3.5 Hz, 2H), 7.89 (s, 1H), 7.72 (d, J=8.3 Hz, 2H), 7.5 (d, J=8.3 Hz, 2H), 7.29 (s, 1H), 6.62-6.69 (m, 1H), 6.25-6.37 (m, 2H), 4.08 (s, 2H), 2.25 (s, 3H), 1.54-1.58 (m, 6H). LC-MS: 98.7%; 460 (M+H).

### Synthesis of Compound 452

### N-methyl-4-{6-[N-methyl(4-fluorobenzene)amido]imidazo[1,2-a]pyrazin-3-yl}benzamide

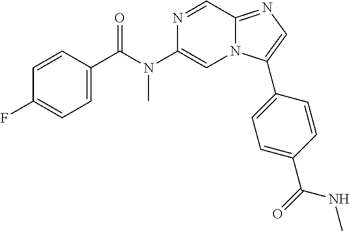 [SMILES: CNC(=O)c1ccc(-c2cnc3cnc(N(C)C(=O)c4ccc(F)cc4)cn23)cc1]

1H NMR (400 MHz, MeOD) δ 8.98 (s, 1H), 8.24 (s, 1H), 7.98 (s, 1H), 7.86 (d, J=8.4, 2H), 7.36 (dd, J=5.3, 8.8, 2H), 7.26 (d, J=8.4, 2H), 7.02 (t, J=8.7, 2H), 3.60-3.35 (m, 3H), 2.88 (s, 3H). HRMS calcd for C₂₂H₁₉FN₅O₂ [M+H]+404.14; found 404.1.

### Synthesis of Compound 453

### N-methyl-N-(4-sulfamoylphenyl)-3-[4-(trifluoromethyl)phenyl]imidazo[1,2-a]pyrazine-6-carboxamide

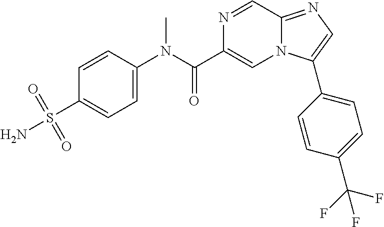 [SMILES: CN(C(=O)c1cn2c(-c3ccc(C(F)(F)F)cc3)cnc2cn1)c1ccc(S(N)(=O)=O)cc1]

The compound was synthesized using a procedure analogous to one use for synthesis of 72 (method II). ¹H NMR (400 MHz, MeOD) δ 8.80 (d, J=10.6 Hz, 2H), 8.10 (s, 1H), 7.93 (d, J=8.3 Hz, 2H), 7.82 (dd, J=8.4 Hz, 17.6 Hz, 4H), 7.42 (d, J=8.6 Hz, 2H), 3.56 (s, 3H). ESI-MS m/z 476 [M+1]. RT: 1.74 min.

### Synthesis of Compound 454

### 4-{6-[(7-fluoro-3,4-dihydro-2H-1,4-benzoxazin-4-yl)carbonyl]imidazo[1,2-a]pyrazin-3-yl}benzamide

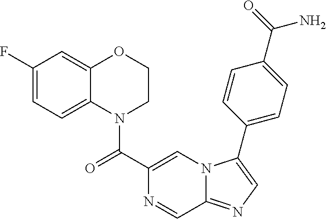 [SMILES: NC(=O)c1ccc(-c2cnc3cnc(C(=O)N4CCOc5cc(F)ccc54)cn23)cc1]

The compound was synthesized using I-74 and an appropriate boronic acid/ester using a procedure similar Suzuki coupling protocol described in 228. ¹H NMR (400 MHz, DMSO-d₆ at 90° C.): 9.07 (s, 1H), 8.88 (s, 1H), 8.17 (s, 1H), 8.06 (d, J=8.4 Hz, 2H), 7.81 (d, J=8.3 Hz, 2H), 7.57-7.60 (m, 2H), 6.72-6.75 (m, 1H), 6.60-6.64 (m, 1H), 4.32-4.34 (t, J=4.6 Hz, 2H), 3.97-3.99 (t, J=4.4 Hz, 2H). LC-MS: 97.7%, 418.14 (M+H).

### Synthesis of Compound 455

### 4-{6-[N-methyl(4-fluorobenzene)amido]imidazo[1,2-a]pyrazin-3-yl}benzamide

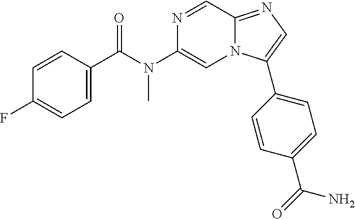 [SMILES: CN(C(=O)c1ccc(F)cc1)c1cn2c(-c3ccc(C(N)=O)cc3)cnc2cn1]

The compound was synthesized using a procedure similar to synthesis of 24. ¹H NMR (400 MHz, MeOD) δ 9.03 (s, 1H), 8.31 (s, 1H), 8.05 (s, 1H), 7.93 (d, J=8.4, 2H), 7.36 (dd, J=6.7, 10.1, 2H), 7.27 (d, J=8.3, 2H), 7.02 (t, J=8.8, 2H), 3.47 (s, 3H). HRMS calcd for C₂₁H₁₇FN₅O₂ [M+H]+390.13; found 390.1.

### Synthesis of Compound 456

### -fluoro-N-methyl-N-(3-{1H-pyrrolo[2,3-b]pyridin-5-yl}imidazo[1,2-a]pyrazin-6-yl)benzamide

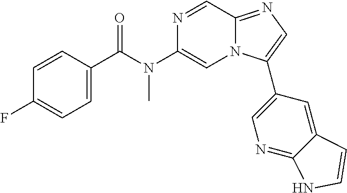 [SMILES: CN(C(=O)c1ccc(F)cc1)c1cn2c(-c3cnc4[nH]ccc4c3)cnc2cn1]

The compound was synthesized using a procedure similar to synthesis of 24. MS m/z 387.0 (M+H)+; r.t.=1.488.

### Synthesis of Compound 457

### 1-{[3-(2,3-dihydro-1-benzofuran-5-yl)imidazo[1,2-a]pyrazin-6-yl]carbonyl}-6-fluoro-1,2,3,4-tetrahydroquinoline

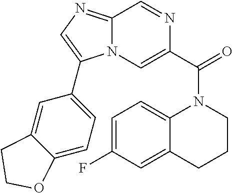 [SMILES: O=C(c1cn2c(-c3ccc4c(c3)CCO4)cnc2cn1)N1CCCc2cc(F)ccc21]

The compound was synthesized using I-72 and an appropriate boronic acid/ester using a procedure similar Suzuki coupling protocol described in 228. LCMS: Rₜ=1.37 min, ES⁺-MS m/z 415 [M+H]⁺.

### Synthesis of Compound 458

### 3-(4-carbamoylphenyl)-N-methyl-N-(5-methylpyridin-2-yl)imidazo[1,2-a]pyrazine-6-carboxamide

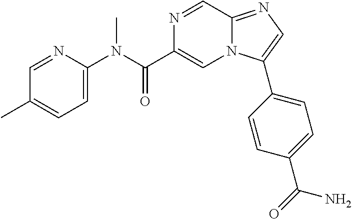 [SMILES: Cc1ccc(N(C)C(=O)c2cn3c(-c4ccc(C(N)=O)cc4)cnc3cn2)nc1]

The compound was synthesized using I-78 and an appropriate boronic acid/ester using a procedure similar Suzuki coupling protocol described in 228. ¹H NMR (400 MHz, METHANOL-d₄) ppm 2.31 (s, 3H) 3.55 (s, 3H) 7.22 (d, J=8.28 Hz, 1H) 7.63 (dd, J=7.78, 2.01 Hz, 1H) 7.78 (d, J=0.10 Hz, 2H) 8.06 (s, 1H) 8.10-8.14 (m, 3H) 8.66 (d, J=1.25 Hz, 1H) 8.86 (d, J=1.26 Hz, 1H); MS (ESI) for C₂₁H₁₈N₆O₂. 387 (M+H)⁺.

### Synthesis of Compound 459

### 4-{6-[(7-fluoro-3,4-dihydro-2H-1,4-benzoxazin-4-yl)carbonyl]imidazo[1,2-a]pyrazin-3-yl}-N-methylbenzamide

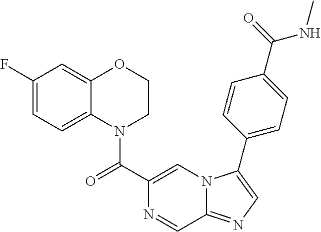 [SMILES: CNC(=O)c1ccc(-c2cnc3cnc(C(=O)N4CCOc5cc(F)ccc54)cn23)cc1]

The compound was synthesized using I-74 and an appropriate boronic acid/ester using a procedure similar Suzuki coupling protocol described in 228. ¹H NMR (400 MHz, METHANOL-d₄) ppm 9.05 (s, 1H) 8.98 (d, J=1.00 Hz, 1H) 8.13 (s, 1H) 8.04 (d, J=8.00 Hz, 2H) 7.83 (d, J=7.80 Hz, 2H) 6.69 (dd, J=9.91, 2.89 Hz, 1H) 6.57 (br. s., 1H) 4.38 (br. s., 2H) 4.03-4.13 (m, 2H) 2.96 (s, 3H); MS (ESI) for C₂₃H₁₈FN₅O₃: 432 (M+H⁺).

### Synthesis of Compound 460

### 6-fluoro-1-{[3-(3-methanesulfonylphenyl)imidazo[1,2-a]pyrazin-6-yl]carbonyl}-1,2,3,4-tetrahydroquinoline

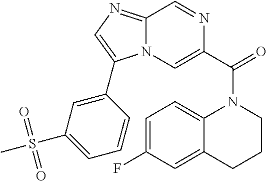 [SMILES: CS(=O)(=O)c1cccc(-c2cnc3cnc(C(=O)N4CCCc5cc(F)ccc54)cn23)c1]

The compound was synthesized using I-72 and an appropriate boronic acid/ester using a procedure similar Suzuki coupling protocol described in 228. LCMS: R₁=1.19 min, ES⁺-MS m/z 451 [M+H]⁺.

### Synthesis of Compound 461

### 3-[6-(4-acetylpiperazin-1-yl)pyridin-3-yl]-N-(4-cyanophenyl)-N-methylimidazo[1,2-a]pyrazine-6-carbo

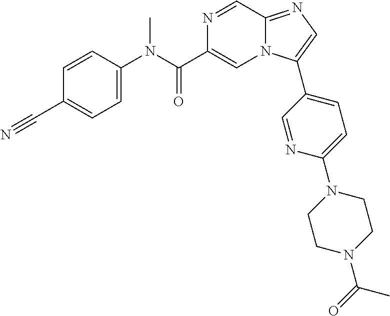 [SMILES: CC(=O)N1CCN(c2ccc(-c3cnc4cnc(C(=O)N(C)c5ccc(C#N)cc5)cn34)cn2)CC1]

The compound was synthesized using I-59 and an appropriate boronic acid/ester using a procedure similar Suzuki coupling protocol described in 228. LCMS: R=0.81 min, ES⁺-MS m/z 481 [M+H]⁺.

### Synthesis of Compound 462

### 6-fluoro-1-{[3-(4-methanesulfonylphenyl)imidazo[1,2-a]pyrazin-6-yl]carbonyl}-1,2,3,4-tetrahydroquinoline

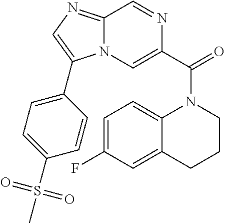 [SMILES: CS(=O)(=O)c1ccc(-c2cnc3cnc(C(=O)N4CCCc5cc(F)ccc54)cn23)cc1]

The compound was synthesized using I-72 and an appropriate boronic acid/ester using a procedure similar Suzuki coupling protocol described in 228. LCMS: Rₜ=1.19 min, ES⁺-MS m/z 451 [M+H]⁺.

### Synthesis of Compound 463

### 1-{[3-(1-ethyl-1H-pyrazol-4-yl)imidazo[1,2-a]pyrazin-6-yl]carbonyl}-6-fluoro-1,2,3,4-tetrahydroquinoline

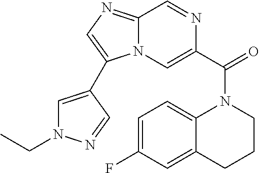 [SMILES: CCn1cc(-c2cnc3cnc(C(=O)N4CCCc5cc(F)ccc54)cn23)cn1]

The compound was synthesized using I-72 and an appropriate boronic acid/ester using a procedure similar Suzuki coupling protocol described in 228. LCMS: Rₜ=1.14 min, ES⁺-MS m/z 391 [M+H]⁺.

### Synthesis of Compound 464

### N-(4-cyanophenyl)-N-methyl-3-[6-(piperazin-1-yl)pyridin-3-yl]imidazo[1,2-a]pyrazine-6-carboxamide

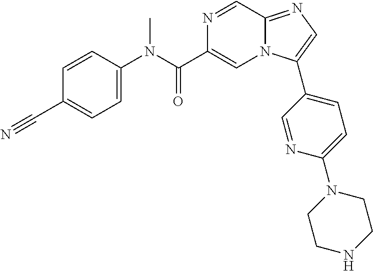 [SMILES: CN(C(=O)c1cn2c(-c3ccc(N4CCNCC4)nc3)cnc2cn1)c1ccc(C#N)cc1]

The compound was synthesized using I-59 and an appropriate boronic acid/ester using a procedure similar Suzuki coupling protocol described in 228. LCMS: Rₜ=0.76 min, ES⁺-MS m/z 439 Da [M+H]⁺.

### Synthesis of Compound 465

### N-(4-cyanophenyl)-3-(furan-2-yl)-N-methylimidazo[1,2-a]pyrazine-6-carboxamide

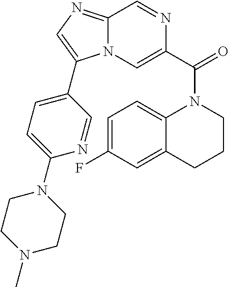 [SMILES: CN1CCN(c2ccc(-c3cnc4cnc(C(=O)N5CCCc6cc(F)ccc65)cn34)cn2)CC1]

The compound was synthesized using I-72 and an appropriate boronic acid/ester using a procedure similar Suzuki coupling protocol described in 228. LCMS: Rₜ=0.86 min, ES⁺-MS m/z 472 Da [M+H]⁺.

### Synthesis of Compound 466

### N-(4-cyanophenyl)-3-(furan-2-yl)-N-methylimidazo[1,2-a]pyrazine-6-carboxamide

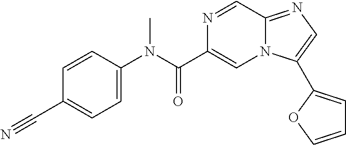 [SMILES: CN(C(=O)c1cn2c(-c3ccco3)cnc2cn1)c1ccc(C#N)cc1]

The compound was synthesized using I-59 and an appropriate boronic acid/ester using a procedure similar Suzuki coupling protocol described in 228. LCMS: Rₜ=1.12 min, ES⁺-MS m/z 344 Da [M+H]⁺.

### Synthesis of Compound 467

### 6-fluoro-1-({3-[2-(piperazin-1-yl)pyridin-4-yl]imidazo[1,2-a]pyrazin-6-yl}carbonyl)-1,2,3,4-tetrahydroquinoline

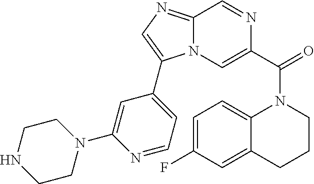 [SMILES: O=C(c1cn2c(-c3ccnc(N4CCNCC4)c3)cnc2cn1)N1CCCc2cc(F)ccc21]

The compound was synthesized using I-72 and an appropriate boronic acid/ester using a procedure similar Suzuki coupling protocol described in 228. LCMS: Rₜ=0.83 min, ES⁺-MS m/z 458 Da [M+H]⁺.

### Synthesis of Compound 468

### N-(4-chlorophenyl)-N-methyl-3-[4-(1,3,4-thiadiazol-2-yl)phenyl]imidazo[1,2-a]pyrazine-6-carboxamide

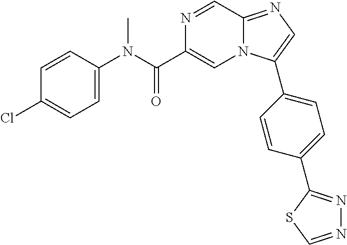 [SMILES: CN(C(=O)c1cn2c(-c3ccc(-c4nncs4)cc3)cnc2cn1)c1ccc(Cl)cc1]

The compound was synthesized using intermediates I-11 and I-61 with a Suzuki coupling protocol described in 228. ¹H NMR (400 MHz, MeOD) δ 9.54 (s, 1H), 8.89-8.74 (m, 2H), 8.33-8.19 (m, 2H), 8.11 (s, 1H), 7.85-7.72 (m, 2H), 7.43-7.17 (m, 4H), 3.52 (s, 3H). ESI-MS m/z 447 [M+1]. RT: 1.75 min.

### Synthesis of Compound 469

### N-methyl-3-[4-(methylcarbamoyl)phenyl]-N-(5-methylpyridin-2-yl)imidazo[1,2-a]pyrazine-6-carboxamide

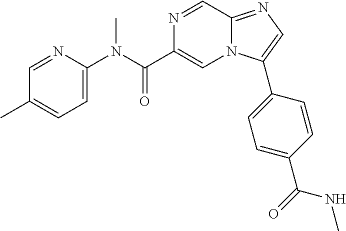

The compound was synthesized using I-78 and an appropriate boronic acid/ester using a procedure similar Suzuki coupling protocol described in 228. ¹H NMR (400 MHz, METHANOL-d₄) ppm 8.86 (d, J=1.26 Hz, 1H) 8.66 (d, J=1.25 Hz, 1H) 8.10 (s, 1H) 8.04-8.07 (m, 3H) 7.78 (d, J=8.28 Hz, 2H) 7.63 (dd, J=8.16, 2.38 Hz, 1H) 7.22 (d, J=8.53 Hz, 1H) 3.55 (s, 3H) 2.98 (s, 3H) 2.31 (s, 3H); MS (ESI) for C₂₂H₂₀N₆O₂: 401 (M+H⁺).

### Synthesis of Compound 470

### N-cyclohexyl-4-{6-[(6-fluoro-1,2,3,4-tetrahydroquinolin-1-yl)carbonyl]imidazo[1,2-a]pyrazin-3-yl}benzamide

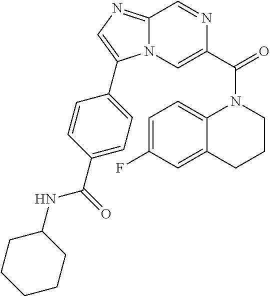 [SMILES: O=C(NC1CCCCC1)c1ccc(-c2cnc3cnc(C(=O)N4CCCc5cc(F)ccc54)cn23)cc1]

The compound was synthesized using I-72 and an appropriate boronic acid/ester using a procedure similar Suzuki coupling protocol described in 228. LCMS: Rₜ=1.43 min, ES⁺-MS m/z 498 Da [M+H]⁺.

### Synthesis of Compound 471

### 4-fluoro-N-methyl-N-[3-(1-methyl-1H-indazol-5-yl)imidazo[1,2-a]pyrazin-6-yl]benzamide

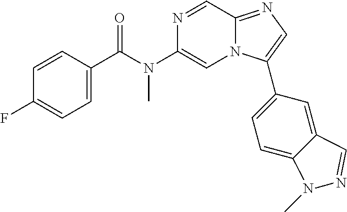 [SMILES: CN(C(=O)c1ccc(F)cc1)c1cn2c(-c3ccc4c(cnn4C)c3)cnc2cn1]

The compound was synthesized using similar to synthesis of 24. 1H NMR (400 MHz, MeOD) δ 8.99 (s, 1H), 8.20 (s, 1H), 8.01 (s, 1H), 7.95 (s, 1H), 7.61 (d, J=8.6, 2H), 7.36-7.29 (m, 2H), 7.13 (d, J=10.2, 1H), 7.02 (dd, J=7.8, 9.7, 2H), 4.04 (s, 3H), 3.42 (d, J=17.2, 3H). HRMS calcd for C₂₂H₁₃FN₆O [M+H]+401.14; found 401.1.

### Synthesis of Compound 472

### 1-[4-(5-{6-[(6-fluoro-1,2,3,4-tetrahydroquinolin-1-yl)carbonyl]imidazo[1,2-a]pyrazin-3-yl}pyridin-2-yl)piperazin-1-yl]ethan-1-one

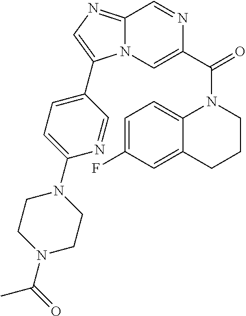 [SMILES: CC(=O)N1CCN(c2ccc(-c3cnc4cnc(C(=O)N5CCCc6cc(F)ccc65)cn34)cn2)CC1]

The compound was synthesized using I-72 and an appropriate boronic acid/ester using a procedure similar Suzuki coupling protocol described in 228. LCMS: Rₜ=0.94 min, ES⁺-MS m/z 500 Da [M+H]⁺.

### Synthesis of Compound 473

### 6-fluoro-1-({3-[3-(morpholin-4-ylmethyl)phenyl]imidazo[1,2-a]pyrazin-6-yl}carbonyl)-1,2,3,4-tetrahydroquinoline

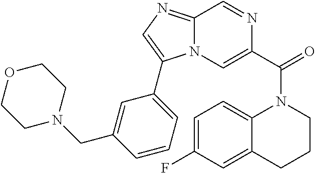 [SMILES: O=C(c1cn2c(-c3cccc(CN4CCOCC4)c3)cnc2cn1)N1CCCc2cc(F)ccc21]

The compound was synthesized using I-72 and an appropriate boronic acid/ester using a procedure similar Suzuki coupling protocol described in 228. LCMS: R₁=0.93 min, ES⁺-MS m/z 472 Da [M+H]⁺.

### Synthesis of Compound 474

### N-(5-methylpyridin-2-yl)-3-[4-(trifluoromethyl)phenyl]imidazo[1,2-a]pyrazine-6-carbox

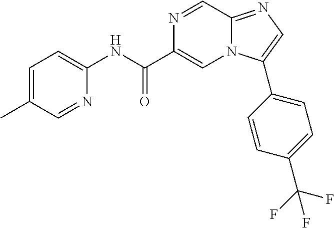 [SMILES: Cc1ccc(NC(=O)c2cn3c(-c4ccc(C(F)(F)F)cc4)cnc3cn2)nc1]

The compound was synthesized using a procedure analogous to one use for synthesis of 78 (method II). ¹H NMR (300 MHz, DMSO-d₆) ppm 10.26 (s, 1H) 9.32 (d, J=1.17 Hz, 1H) 9.13 (d, J=1.17 Hz, 1H) 8.34 (s, 1H) 8.24 (s, 1H) 8.16 (d, J=8.20 Hz, 1H) 7.97-8.10 (m, 4H) 7.71-7.78 (m, 1H) 2.29 (s, 3H); MS (ESI) for C₂₀H₁₄F₃N₅O: 398 (M+H)⁺.

### Synthesis of Compound 475

### N-(4-ethylpyridin-2-yl)-N-methyl-3-[4-(trifluoromethyl)phenyl]imidazo[1,2-a]pyrazine-6-carboxamide

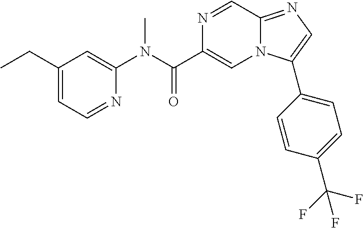 [SMILES: CCc1ccnc(N(C)C(=O)c2cn3c(-c4ccc(C(F)(F)F)cc4)cnc3cn2)c1]

The compound was synthesized using a procedure analogous to one use for synthesis of 72 (method II). ¹H NMR (300 MHz, DMSO-d₆) ppm 9.46 (d, J=1.46 Hz, 1H) 9.19 (d, J=1.17 Hz, 1H) 8.17-8.27 (m, 2H) 7.94-8.14 (m, 5H) 6.73 (dd, J=6.74, 2.05 Hz, 1H) 3.82 (s, 3H) 2.62 (q, J=7.33 Hz, 2H) 1.11-1.26 (m, 3H); MS (ESI) for C₂₂H₁₈F₃N₅O: 426 (M+H⁺).

### Synthesis of Compound 476

### N-(4-cyanophenyl)-N-methyl-3-{4-[5-(methylamino)-1,3,4-thiadiazol-2-yl]phenyl}imidazo[1,2-a]pyrazine-6-carboxamide

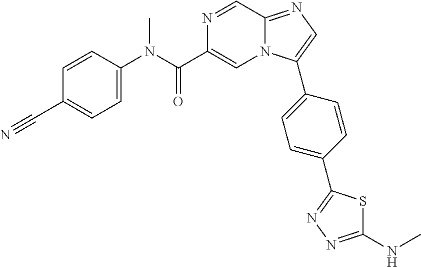 [SMILES: CNc1nnc(-c2ccc(-c3cnc4cnc(C(=O)N(C)c5ccc(C#N)cc5)cn34)cc2)s1]

The compound was synthesized using I-59 and an appropriate boronic acid/ester using a procedure similar Suzuki coupling protocol described in 228. 1H NMR (400 MHz, DMSO) δ 8.91 (d, J=1.4 Hz, 1H), 8.82 (d, J=1.3 Hz, 1H), 8.21 (s, 1H), 8.09-7.94 (m, 3H), 7.90-7.80 (m, 2H), 7.80-7.71 (m, 2H), 7.55-7.38 (m, 2H), 3.48 (s, 3H), 2.97 (d, J=4.8 Hz, 3H). MS m/z 467.1 (M+H)+; r.t.=1.287.

### Synthesis of Compound 477

### 4-(6-{[(4-chlorophenyl)(methyl)amino]methyl}imidazo[1,2-a]pyrazin-3-yl)benzamide

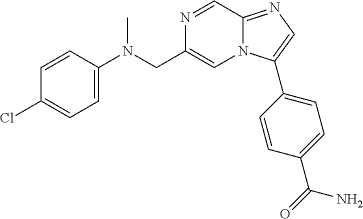 [SMILES: CN(Cc1cn2c(-c3ccc(C(N)=O)cc3)cnc2cn1)c1ccc(Cl)cc1]

The compound was synthesized using I-63 and an appropriate boronic acid/ester using a procedure similar Suzuki coupling protocol described in 228. ¹H NMR (400 MHz, DMSO) δ 9.13 (d, J=1.3, 1H), 8.52 (s, 1H), 8.12 (s, 2H), 8.05 (d, J=8.4, 2H), 7.74 (d, J=8.4, 2H), 7.51 (s, 1H), 7.16 (d, J=9.1, 2H), 6.84 (d, J=9.1, 2H), 4.68 (s, 2H), 3.06 (s, 3H); MS m/z 392.1 (M+H)+; r.t.=1.459.

### Synthesis of Compound 478

### N-(4-fluorophenyl)-N-methyl-3-{4-[5-(methylamino)-1,3,4-oxadiazol-2-yl]phenyl}imidazo[1,2-a]pyrazine-6-carboxamide

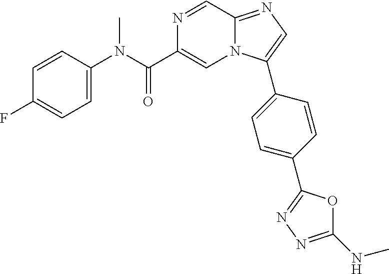 [SMILES: CNc1nnc(-c2ccc(-c3cnc4cnc(C(=O)N(C)c5ccc(F)cc5)cn34)cc2)o1]

The compound was synthesized using I-64 and an appropriate boronic acid/ester using a procedure similar Suzuki coupling protocol described in 228. ¹H NMR (400 MHz, MeOD) δ 8.93-8.57 (m, 3H), 8.18-8.00 (m, 2H), 7.84-7.63 (m, 2H), 7.39-7.17 (m, 2H), 7.16-6.95 (m, 2H), 3.51 (s, 3H), 3.03 (s, 3H). ESI-MS m/z 444 [M+1]. RT: 1.54 min.

### Synthesis of Compound 479

### 4-[({3-[4-(difluoromethoxy)phenyl]imidazo[1,2-a]pyrazin-6-yl}methyl)(methyl)amino]benzonitrile

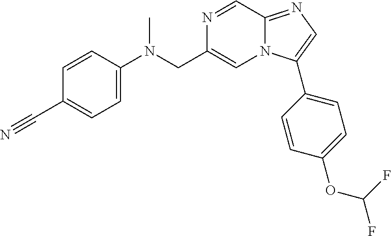 [SMILES: CN(Cc1cn2c(-c3ccc(OC(F)F)cc3)cnc2cn1)c1ccc(C#N)cc1]

The compound was synthesized using I-15 and an appropriate boronic acid/ester using a procedure similar Suzuki coupling protocol described in 228. 1H NMR (400 MHz, DMSO) δ 9.11 (d, J=1.3 Hz, 1H), 8.59 (d, J=1.0 Hz, 1H), 8.04 (s, 1H), 7.91-7.67 (m, 2H), 7.65-7.46 (m, 2H), 7.44-7.31 (m, 2H), 6.96 (dd, J=5.0, 6.9, 2H), 4.78 (s, 2H), 3.15 (s, 3H). M/Z=406.1 (M+1), r.t=1.61 mins.

### Synthesis of Compound 480

### N-(4-chlorophenyl)-N-methyl-3-(1-methyl-1H-indazol-5-yl)imidazo[1,2-a]pyrazine-6-carboxamide

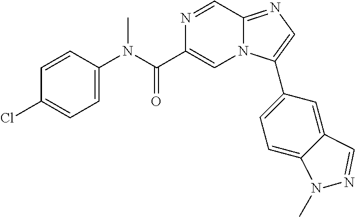 [SMILES: CN(C(=O)c1cn2c(-c3ccc4c(cnn4C)c3)cnc2cn1)c1ccc(Cl)cc1]

The compound was synthesized using I-11 and an appropriate boronic acid/ester using a procedure similar Suzuki coupling protocol described in 228. MS m/z 417.1 (M+H)+; r.t.=1.541.

### Synthesis of Compound 481

### N-(4-cyanophenyl)-N-methyl-3-[4-(1H-pyrazol-5-yl)phenyl]imidazo[1,2-a]pyrazine-6-carboxamide

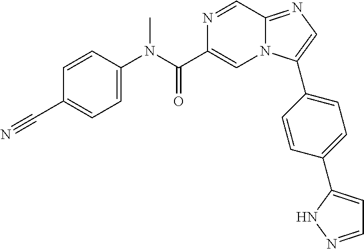 [SMILES: CN(C(=O)c1cn2c(-c3ccc(-c4ccn[nH]4)cc3)cnc2cn1)c1ccc(C#N)cc1]

The compound was synthesized using I-59 and an appropriate boronic acid/ester using a procedure similar Suzuki coupling protocol described in 228. 1H NMR (400 MHz, DMSO) δ 8.89 (d, J=1.4 Hz, 1H), 8.81 (d, J=1.2 Hz, 1H), 8.17 (s, 1H), 8.06 (s, 2H), 7.81-7.73 (m, 5H), 7.54-7.43 (m, 2H), 6.87 (d, J=2.2 Hz, 1H), 3.45 (s, 3H). M/Z=420.2 (M+1), r.t=1.34 mins.

### Synthesis of Compound 482

### methyl N-(4-{6-[(4-cyanophenyl)(methyl)carbamoyl]imidazo[1,2-a]pyrazin-3-yl}phenyl)carbamate

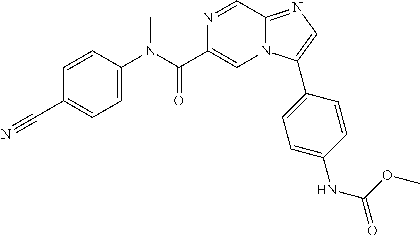 [SMILES: COC(=O)Nc1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. M/Z=427.2 (M+1), r.t=1.43 mins.

### Synthesis of Compound 483

### N-(4-chlorophenyl)-N-methyl-3-{4-[5-(methylamino)-1,3,4-oxadiazol-2-yl]phenyl}imidazo[1,2-a]pyrazine-6-carboxamide

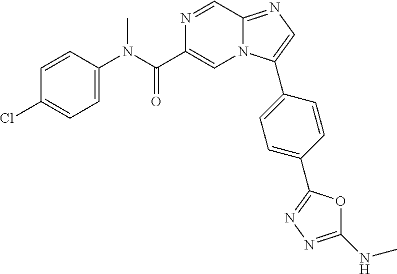 [SMILES: CNc1nnc(-c2ccc(-c3cnc4cnc(C(=O)N(C)c5ccc(Cl)cc5)cn34)cc2)o1]

The compound was synthesized using I-11 and I-60 using a procedure similar Suzuki coupling protocol described in 228. ¹H NMR (400 MHz, CD2Cl2) δ 8.76 (s, 2H), 8.22-8.07 (m, 2H), 7.97 (s, 1H), 7.67 (d, J=8.4 Hz, 2H), 7.30 (d, J=8.6 Hz, 2H), 7.14 (d, J=8.3 Hz, 2H), 3.51 (s, 3H), 3.14 (d, J=5.2 Hz, 3H). ESI-MS m/z 460 [M+H]+. RT: 1.55 min.

### Synthesis of Compound 484

### N-(4-cyanophenyl)-N-methyl-3-[4-(1H-pyrazol-3-yl)phenyl]imidazo[1,2-a]pyrazine-6-carboxamide

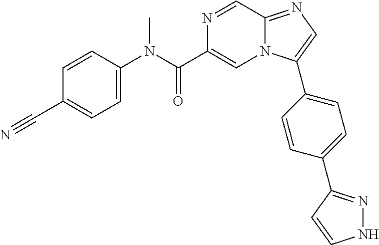 [SMILES: CN(C(=O)c1cn2c(-c3ccc(-c4cc[nH]n4)cc3)cnc2cn1)c1ccc(C#N)cc1]

The compound was synthesized using I-59 and an appropriate boronic acid/ester using a procedure similar Suzuki coupling protocol described in 228. ¹H NMR (400 MHz, DMSO) δ 8.88 (d, J=1.3, 1H), 8.80 (d, J=1.1, 1H), 8.17 (d, J=9.1, 1H), 8.03 (dd, J=7.8, 26.7, 2H), 7.92-7.53 (m, 5H), 7.51-7.41 (m, 2H), 6.86 (d, J=1.9, 1H), 3.51-3.43 (m, 3H); MS m/z 420.1 (M+H)+; r.t.=1.327.

### Synthesis of Compound 485

### N-(4-cyanophenyl)-3-(4-acetamidophenyl)-N-methylimidazo[1,2-a]pyrazine-6-carboxamide

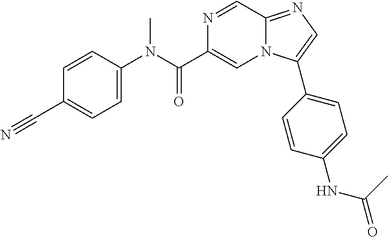 [SMILES: CC(=O)Nc1ccc(-c2cnc3cnc(C(=O)N(C)c4ccc(C#N)cc4)cn23)cc1]

The compound was synthesized using I-59 and an appropriate boronic acid/ester using a procedure similar Suzuki coupling protocol described in 228. M/Z 411 [M+1]+. RT: 1.21 min.

### Synthesis of Compound 486

### 3-(4-carbamoylphenyl)-N-(4-chlorophenyl)-N-methylimidazo[1,2-a]pyrazine-6-carboxamide

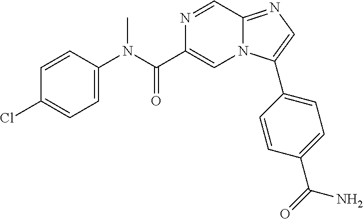 [SMILES: CN(C(=O)c1cn2c(-c3ccc(C(N)=O)cc3)cnc2cn1)c1ccc(Cl)cc1]

The compound was synthesized using I-11 and an appropriate boronic acid/ester using a procedure similar Suzuki coupling protocol described in 228. ¹H NMR (400 MHz, DMSO) δ 8.86 (s, 1H), 8.81 (d, J=1.2, 1H), 8.19 (s, 1H), 8.16 (s, 1H), 8.09 (d, J=8.3, 2H), 7.78 (d, J=8.3, 2H), 7.55 (s, 1H), 7.33 (q, J=8.8, 4H), 3.42 (s, 3H); MS m/z 406.0 (M+H)+; r.t.=1.319.

### Synthesis of Compound 487

### N-(4-cyanophenyl)-N-methyl-3-(1-methyl-1H-indazol-5-yl)imidazo[1,2-a]pyrazine-6-carboxamide

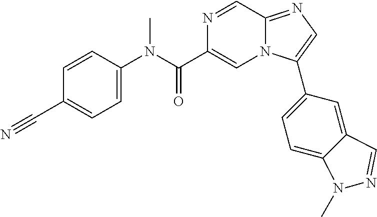 [SMILES: CN(C(=O)c1cn2c(-c3ccc4c(cnn4C)c3)cnc2cn1)c1ccc(C#N)cc1]

The compound was synthesized using I-59 and an appropriate boronic acid/ester using a procedure similar Suzuki coupling protocol described in 228. 1H NMR (400 MHz, DMSO) δ 8.78 (d, J=1.4 Hz, 1H), 8.72 (d, J=1.3 Hz, 1H), 8.15 (d, J=0.9 Hz, 1H), 8.06 (d, J=1.8 Hz, 2H), 7.84 (d, J=8.8 Hz, 1H), 7.74-7.68 (m, 2H), 7.64 (dd, J=1.6 Hz, 8.7 Hz, 1H), 7.49-7.38 (m, 2H), 4.04 (s, 3H), 3.40 (s, 3H). M/Z=408.1 (M+1), r.t=1.58 mins.

### Synthesis of Compound 488

### N-[3-(6-acetamidopyridin-3-yl)imidazo[1,2-a]pyrazin-6-yl]-4-fluoro-N-methylbenzamide

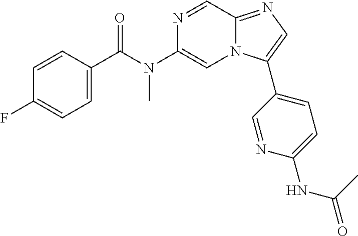 [SMILES: CC(=O)Nc1ccc(-c2cnc3cnc(N(C)C(=O)c4ccc(F)cc4)cn23)cn1]

The compound was synthesized using a procedure similar to synthesis of 24. MS m/z 405.1 (M+H)+; r.t.=1.437.

### Synthesis of Compound 489

### N-(6-chloropyridin-3-yl)-N-methyl-3-(1-methyl-1H-indazol-5-yl)imidazo[1,2-a]pyrazine-6-carboxamide

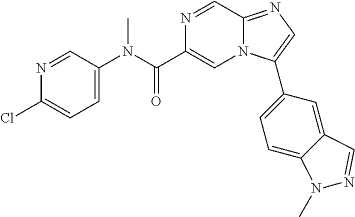 [SMILES: CN(C(=O)c1cn2c(-c3ccc4c(cnn4C)c3)cnc2cn1)c1ccc(Cl)nc1]

The compound was synthesized using I-57 and appropriate boronic acid/ester using a procedure similar Suzuki coupling protocol described in 228. MS m/z 418.1 (M+H)+; r.t.=1.337.

### Synthesis of Compound 490

### N-(4-fluoropyridin-2-yl)-N-methyl-3-[4-(trifluoromethyl)phenyl]imidazo[1,2-a]pyrazine-6-carboxamide

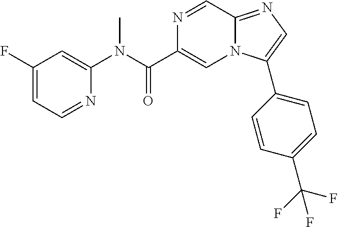 [SMILES: CN(C(=O)c1cn2c(-c3ccc(C(F)(F)F)cc3)cnc2cn1)c1cc(F)ccn1]

The compound was synthesized using I-40 following a procedure analogous to one use for synthesis of 72 (method II). MS m/z 416.1 (M+H)+; r.t.=1.638.

### Synthesis of Compound 491

### N-methyl-N-(5-methylpyridin-2-yl)-3-[4-(trifluoromethyl)phenyl]imidazo[1,2-a]pyrazine-6-carboxamide

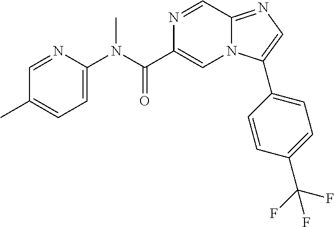 [SMILES: Cc1ccc(N(C)C(=O)c2cn3c(-c4ccc(C(F)(F)F)cc4)cnc3cn2)nc1]

The compound was synthesized using a procedure analogous to one use for synthesis of 78 (method II). ¹H NMR (400 MHz, CDCl3) δ 8.85 (d, J=1.4, 1H), 8.66 (d, J=1.3, 1H), 8.04 (d, J=2.2, 1H), 7.92 (s, 1H), 7.81 (d, J=8.2, 2H), 7.70 (d, J=8.1, 2H), 7.45 (dd, J=2.1, 8.1, 1H), 7.04 (d, J=8.1, 1H), 3.56 (s, 3H), 2.28 (s, 3H); MS m/z 412.2 (M+H)+; r.t.=1.456.

### Synthesis of Compound 492

### N-(2-methoxypyridin-3-yl)-N-methyl-3-[4-(trifluoromethyl)phenyl]imidazo[1,2-a]pyrazine-6-carboxamide

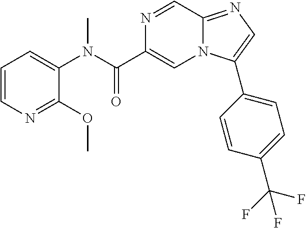 [SMILES: COc1ncccc1N(C)C(=O)c1cn2c(-c3ccc(C(F)(F)F)cc3)cnc2cn1]

The compound was synthesized using I-46 following a procedure analogous to one use for synthesis of 72 (method II). MS m/z 428.1 (M+H)+; r.t.=1.616.

### Synthesis of Compound 493

### N-(4-cyanophenyl)-N-methyl-3-[4-(1,3,4-thiadiazol-2-yl)phenyl]imidazo[1,2-a]pyrazine-6-carboxamide

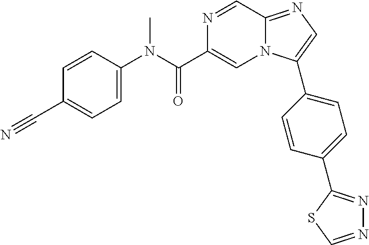 [SMILES: CN(C(=O)c1cn2c(-c3ccc(-c4nncs4)cc3)cnc2cn1)c1ccc(C#N)cc1]

The compound was synthesized using I-59 and I-60 using a procedure similar Suzuki coupling protocol described in 228. ¹H NMR (400 MHz, MeOD) δ 9.62-9.46 (m, 2H), 8.37-8.23 (m, 2H), 8.22-8.06 (m, 2H), 7.97-7.83 (m, 2H), 7.76-7.62 (m, 2H), 7.50-7.39 (m, 2H), 3.57 (s, 3H). ESI-MS m/z 438 [M+H]+. RT: 1.65 min.

### Synthesis of Compound 494

### (1,1-dioxido-2H-benzo[b][1,4]thiazin-4(3H)-yl)(3-(4-(trifluoromethyl)phenyl)imidazo[1,2-a]pyrazin-6-yl)methanone

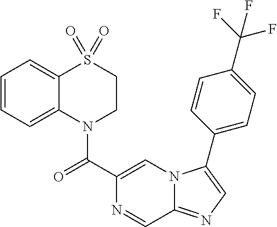 [SMILES: O=C(c1cn2c(-c3ccc(C(F)(F)F)cc3)cnc2cn1)N1CCS(=O)(=O)c2ccccc21]

The compound was synthesized using a procedure analogous to one use for synthesis of 72 (method II). MS m/z 473.1 (M+H)+; r.t.=1.646.

### Synthesis of Compound 495

### 4-{6-[N-methyl(4-cyanobenzene)amido]imidazo[1,2-a]pyrazin-3-yl}benzamide

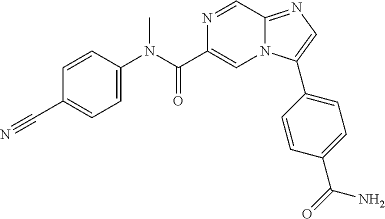 [SMILES: CN(C(=O)c1cn2c(-c3ccc(C(N)=O)cc3)cnc2cn1)c1ccc(C#N)cc1]

The compound was synthesized using a procedure similar to synthesis of 24. MS m/z 473.1 (M+H)+; r.t.=1.646.

### Synthesis of Compound 496

### N-(2-chloro-1,3-thiazol-5-yl)-N-methyl-3-[4 (trifluoromethyl)phenyl]imidazo[1,2-a]pyrazine-6-carboxamide

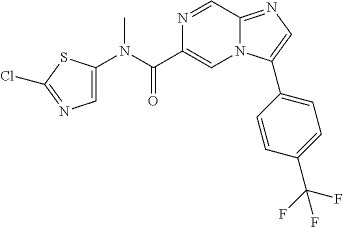 [SMILES: CN(C(=O)c1cn2c(-c3ccc(C(F)(F)F)cc3)cnc2cn1)c1cnc(Cl)s1]

The compound was synthesized using a procedure analogous to one use for synthesis of 72 (method III). MS m/z 438.0 (M+H)+; r.t.=1.779.

### Synthesis of Compound 497

### 4-({3-[4-(trifluoromethyl)phenyl]imidazo[1,2-a]pyrazin-6-yl}carbonyl)-1,2,3,4-tetrahydroquinoxalin-2-one

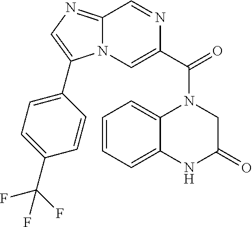 [SMILES: O=C1CN(C(=O)c2cn3c(-c4ccc(C(F)(F)F)cc4)cnc3cn2)c2ccccc2N1]

The compound was synthesized using a procedure analogous to one use for synthesis of 72 (method II). MS m/z 438.0 (M+H)+; r.t.=1.548.

### Synthesis of Compound 498

### N-methyl-N-{3-[4-(trifluoromethyl)phenyl]imidazo[1,2-a]pyrazin-6-yl}pyrimidine-5-carboxamide

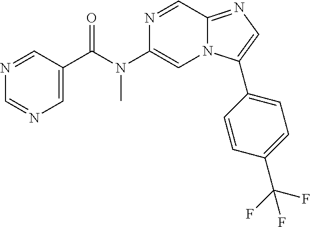 [SMILES: CN(C(=O)c1cncnc1)c1cn2c(-c3ccc(C(F)(F)F)cc3)cnc2cn1]

The compound was synthesized using a procedure similar to synthesis of 24. MS m/z 399.1 (M+H)+; r.t.=1.671.

### Synthesis of Compound 499

### N-methyl-N-(6-phenylpyridin-3-yl)-3-[4-(trifluoromethyl)phenyl]imidazo[1,2-a]pyrazine-6-carboxamide

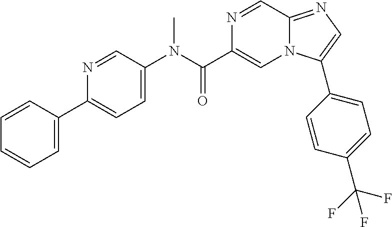 [SMILES: CN(C(=O)c1cn2c(-c3ccc(C(F)(F)F)cc3)cnc2cn1)c1ccc(-c2ccccc2)nc1]

The compound was synthesized using a procedure analogous to one use for synthesis of 72 (method II) using I-53. MS m/z 474.1 (M+H)+; r.t.=1.749.

### Synthesis of Compound 500

### N-methyl-N,3-bis[4-(trifluoromethyl)phenyl]imidazo[1,2-a]pyrazine-6-carboxamide

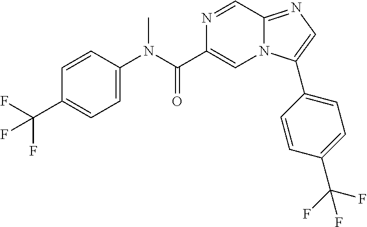 [SMILES: CN(C(=O)c1cn2c(-c3ccc(C(F)(F)F)cc3)cnc2cn1)c1ccc(C(F)(F)F)cc1]

The compound was synthesized using a procedure analogous to one use for synthesis of 72 (method III). MS m/z 465.1 (M+H)+; r.t.=2.260.

### Synthesis of Compound 501

### N-(4-methanesulfonylphenyl)-N-methyl-3-[4-(trifluoromethyl)phenyl]imidazo[1,2-a]pyrazine-6-carboxamide

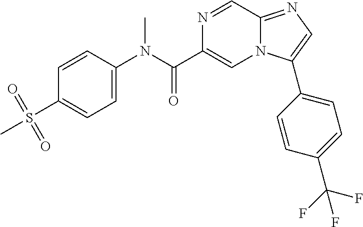 [SMILES: CN(C(=O)c1cn2c(-c3ccc(C(F)(F)F)cc3)cnc2cn1)c1ccc(S(C)(=O)=O)cc1]

The compound was synthesized using I-64 and an appropriate boronic acid/ester using a procedure similar Suzuki coupling protocol described in 228. MS m/z 475.1 (M+H)+; r.t.=1.800.

### Synthesis of Compound 502

### N-(3-chloro-4-cyanophenyl)-N-methyl-3-[4-(trifluoromethyl)phenyl]imidazo[1,2-a]pyrazine-6-carboxamide

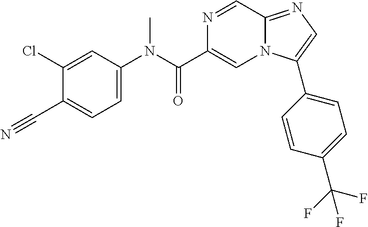 [SMILES: CN(C(=O)c1cn2c(-c3ccc(C(F)(F)F)cc3)cnc2cn1)c1ccc(C#N)c(Cl)c1]

The compound was synthesized using a procedure analogous to one use for synthesis of 72 (method II). MS m/z 456.0 (M+H)+; r.t.=2.150.

### Synthesis of Compound 503

### N-(4-cyano-2-methoxyphenyl)-N-methyl-3-[4-(trifluoromethyl)phenyl]imidazo[1,2-a]pyrazine-6-carboxamide

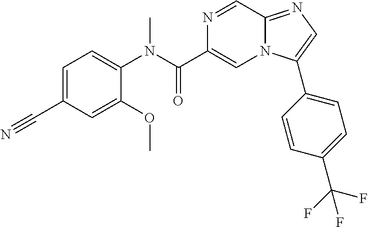 [SMILES: COc1cc(C#N)ccc1N(C)C(=O)c1cn2c(-c3ccc(C(F)(F)F)cc3)cnc2cn1]

The compound was synthesized using a procedure analogous to one use for synthesis of 72 (method II). MS m/z 452.1 (M+H)+; r.t.=1.690.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. A compound of Formula I, 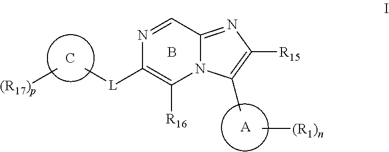
or a pharmaceutical acceptable salt, tautomer or stereoisomer thereof, wherein
n is 0, 1 or 2;
p is 0, 1, 2 or 3;
Ring A is selected from the group consisting of C₆₋₁₀aryl, C₅₋₁₀heteroaryl and fused bicyclyls comprising a C₅₋₆heterocycloalkyl fused to a phenyl;
Ring B represents the imidazo[1,2-a]pyrazine fused ring depicted in Formula 1;
Ring C is selected from the group consisting of phenyl, C₅₋₁₀heteroaryl, C₅₋₆cycloalkyl, C₅₋₆heterocycloalkyl, and fused bicyclyl comprising a C₅₋₆heterocycloalkyl fused to a phenyl;
L is selected from the group consisting of *—CH₂N(R₂)—, *—C(O)—, *—C(O)N(R₂)—, *—C(O)N(R₂)C(R₃ₐ)(R_{3b})—, *—N(R₂)C(R₃ₐ)(R_{3b})—, *—N(R₂)C(O)—, *—N(R₂)SO₂—, and C₁₋₆alkylene,
wherein
* represents the point of attachment of L to Ring B;
R₂ is selected from the group consisting of hydrogen, C₁₋₄alkyl and R₀—C₁₋₄alkylene, wherein R₀ is selected from the group consisting of C₁₋₄alkyl, C₁₋₄ alkoxy, amino, C₁₋₄alkylamino, C₅₋₆heteroaryl and C₅₋₆heterocycloalkyl, wherein the C₅₋₆heteroaryl and C₅₋₆heterocycloalkyl of R₀ are each unsubstituted or substituted with 1 to 2 substituents independently selected from the group consisting of C₁₋₄alkyl, halo and amino;
R₃ₐ and R_{3b} are each independently selected from the group consisting of hydrogen and C₁₋₄alkyl, or R₃ₐ and R_{3b} is taken together with the carbon to which both attached to form a cyclopropyl;
each R₁ is independently selected from the group consisting of halo, cyano, —OR₄, —C(O)R₅, —C(O)NR₆R₇, —NR₈R₉, —NHC(O)R₁₀, —NHSO₂R₁₁, —SO₂R₁₂, C₁₋₆alkyl, phenyl, C₅₋₉heteroaryl, and C₄₋₆heterocycloalkyl, wherein
R₄ is C₁₋₆alkyl or phenyl, wherein the C₁₋₆alkyl is unsubstituted or substituted by 1 to 3 substituents independently selected from the group consisting of halo, cyano, C₁₋₄alkyl, amino, di-C₁₋₄alkylamino, and —C(O)NH₂);
R₅ is hydrogen, C₁₋₆alkyl or C₁₋₆alkoxy;
R₆, R₈ and R₁₁ are each independently hydrogen or C₁₋₄alkyl;
R₇ and R₉ are each independently selected from the group consisting of hydrogen, C₁₋₄alkyl, C₁₋₄alkoxy, and C₃₋₆cycloalkyl, wherein the C₁₋₄alkyl is unsubstituted or substituted with 1 to 3 substituents independently selected from the group consisting of amino, C₁₋₄alkyl, C₁₋₄alkylamino, di-C₁₋₄alkylamino, C₁₋₄ alkoxycarbonylamino, and C₅₋₆heterocycloalkyl;
R₁₀ is C₁₋₆alkyl, C₁₋₆alkoxy or C₃₋₆cycloalkyl, wherein the C₁₋₆alkyl of R₁₀ is unsubstituted or substituted by 1-2 substituents independently selected from amino and C₃₋₆cycloalkyl, and the C₃₋₆cycloalkyl of R₁₀ is unsubstituted or substituted by 1 to 2 substituents independently selected from the group consisting of amino and amino-C₁₋₄alkyl,
R₁₂ is C₁₋₄alkyl, amino or C₁₋₄alkylamino;
the C₁₋₆alkyl of R₁ is unsubstituted or substituted with 1-3 substituents independently selected from the group consisting of halo, cyano, methoxy, amino, C₁₋₄alkylamino, C₅₋₆cycloalkyl, and phenyl; and
the phenyl, C₅₋₆heteroaryl and C₃₋₆heterocycloalkyl of R₁ are each independently unsubstituted or substituted with 1 to 2 substituents independently selected from the group consisting of C₁₋₄alkyl, amino, C₁₋₄alkylamino, —C(O)CH₃, and benzyl;
R₁₅ and R₁₆ are each independently hydrogen, C₁₋₄alkyl or haloC₁₋₄alkyl;
each R₁₇ is independently selected from the group consisting of cyano, halo, oxo, OR₁₈, —C(O)R₁₉, —NR₂₀R₂₁, —SO₂R₂₂, —SO₂NHR₂₃, C₁₋₄alkyl, phenyl, C₅₋₉heteroaryl, C₃₋₆cycloalkyl and C₄₋₆heterocycloalkyl, wherein
R₁₈ is selected from the group consisting of hydrogen, C₁₋₄alkyl, haloC₁₋₄alkyl and phenyl;
R₁₉ is selected from the group consisting of hydrogen, C₁₋₄alkyl, amino, and C₁₋₄alkylamino;
R₂₀, R₂₁ and R₂₂ are each independently hydrogen or C₁₋₄alkyl;
R₂₃ is hydrogen, C₁₋₄alkyl, or C₅₋₆heteroaryl;
the C₁₋₄alkyl of R₁₇ is unsubstituted or substituted with 1-3 substituents independently selected from the group consisting of halo, C₁₋₄alkoxy and amino; and
the phenyl, C₅₋₉heteroaryl, C₃₋₆cycloalkyl and C₄₋₆heterocycloalkyl of R₁₇ are each independently unsubstituted or substituted by 1 to 2 substituents independently selected from the group consisting of C₁₋₄alkyl, halo-C₁₋₄alkyl, C₁₋₄alkoxy-C₁₋₄alkyl, and C₁₋₄alkoxy.

2. The compound of claim 1, wherein L is selected from the group consisting of *—CH₂N(R₂)—, *—C(O)—, *—C(O)N(R₂)—, *—C(O)N(R₂)C(R₃ₐ)(R_{3b})—, *—N(R₂)C(O)—, *—N(R₂)SO₂—, and C₁₋₆alkylene, wherein
* represents the point of attachment of L to Ring B;
R₂ is C₁₋₄alkyl;
R₃ₐ and R_{3b} are each independently selected from the group consisting of hydrogen and C₁₋₄alkyl, or R₃ₐ and R_{3b} is taken together with the carbon to which both attached to form a cyclopropyl.

3. The compound according to claim 1, wherein Ring A is C₆₋₁₀aryl or C₅₋₁₀heteroaryl.

4. The compound according to claim 1, wherein Ring C is selected from the group consisting of phenyl, C₅₋₁₀heteroaryl and C₅₋₁₀heterocycloalkyl.

5. The compound according to claim 1, wherein each R₁ is independently selected from the group consisting of trifluoromethyl, —C(O)NH₂, —C(O)NHCH₃, —C(O)NH(CH₂)₂N(CH₃)₂, and —NHC(O)CH(NH₂)-cycloalkyl.

6. The compound according to claim 1, wherein each R₁₇ is independently selected from the group consisting of fluoro, chloro, bromo, cyano, methyl, ethyl, t-butyl, trifluoromethyl, methoxymethyl, aminomethyl, methoxy, ethoxy, isopropoxy, difluoromethoxy, trifluoromethoxy, phenoxy, oxo, dimethylamino, methylsulfonyl, and aminocarbonyl.

7. The compound of claim 1, wherein the compound is of Formula 1A: 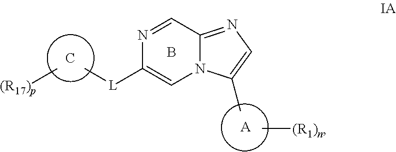
wherein
n is 1;
p is 1;
L is *—C(O)N(R₂)— or *—CH₂N(R₂)—, wherein
* denotes the point of attachment of L to Ring B,
R₂ is C₁₋₄alkyl or R₀—C₁₋₄alkylene, wherein R₀ is selected from the group consisting of C₁₋₄alkoxy, amino, C₁₋₄alkylamino, C₅₋₆heteroaryl and C₅₋₆heterocycloalkyl, wherein the C₅₋₆heteroaryl and C₅₋₆heterocycloalkyl are each independently unsubstituted or substituted by 1-2 substituents independently selected from the group consisting of C₁₋₄alkyl, halo, amino, and oxo;
Ring A is phenyl or pyridyl;
Ring C is phenyl or pyridyl;
each R₁ is trifluoromethyl, *—C(O)NH₂, or *—C(O)NHCH₃;
each R₁₇ is chloro, fluoro, or cyano.

8. The compound of claim 1 selected from the group consisting of:
N-(4-cyanophenyl)-3-(6-(3-(dimethylamino)propoxy)pyridin-3-yl)-N-methylimidazo[1,2-a]pyrazine-6-carboxamide;
4-(((3-(4-(1H-pyrazol-5-yl)phenyl)imidazo[1,2-a]pyrazin-6-yl)methyl)(methyl)amino)benzonitrile;
N-((3-(4-(1H-pyrazol-5-yl)phenyl)imidazo[1,2-a]pyrazin-6-yl)methyl)-4-fluoro-N-methylaniline;
4-(((3-(4-chlorophenyl)imidazo[1,2-a]pyrazin-6-yl)methyl)(methyl)amino)benzonitrile;
N-((3-(4-(1H-pyrazol-5-yl)phenyl)imidazo[1,2-a]pyrazin-6-yl)methyl)-4-chloro-N-methylaniline;
N-(5-chloro-3-fluoropyridin-2-yl)-N-methyl-3-(4-(trifluoromethyl)phenyl)imidazo[1,2-a]pyrazine-6-carboxamide;
5-methanesulfonyl-1-({3-[4-(trifluoromethyl)phenyl]imidazo[1,2-a]pyrazin-6-yl}carbonyl)-2,3-dihydro-1H-indole;
4-fluoro-N-methyl-N-((3-(1-methyl-1H-indazol-5-yl)imidazo[1,2-a]pyrazin-6-yl)methyl)aniline;
N-((3-(4-(1H-pyrazol-1-yl)phenyl)imidazo[1,2-a]pyrazin-6-yl)methyl)-4-chloro-N-methylaniline;
N-((3-(4-(1H-pyrazol-1-yl)phenyl)imidazo[1,2-a]pyrazin-6-yl)methyl)-4-chloro-N-methylaniline;
3-(4-carbamoylphenyl)-N-(4-fluorophenyl)-N-methylimidazo[1,2-a]pyrazine-6-carboxamide;
N-methyl-N-(1-methyl-1H-benzo[d][1,2,3]triazol-5-yl)-3-(4-(trifluoromethyl)phenyl)imidazo[1,2-a]pyrazine-6-carboxamide;
N-(4-chlorophenyl)-N-methyl-3-[4-(1H-pyrazol-5-yl)phenyl]imidazo[1,2-a]pyrazine-6-carboxamide;
N-(1,3-benzothiazol-2-yl)-N-methyl-3-[4-(trifluoromethyl)phenyl]imidazo[1,2-a]pyrazine-6-carboxamide;
[4-(5-amino-1,3,4-oxadiazol-2-yl)phenyl]-N-(4-chlorophenyl)-N-methylimidazo[1,2-a]pyrazine-6-carboxamide;
N-(3-methoxypyridin-2-yl)-N-methyl-3-[4-(trifluoromethyl)phenyl]imidazo[1,2-a]pyrazine-6-carboxamide;
3-(4-chlorophenyl)-N-(4-methanesulfonylphenyl)-N-methylimidazo[1,2-a]pyrazine-6-carboxamide;
N-{[3-(4-chlorophenyl)imidazo[1,2-a]pyrazin-6-yl]methyl}-4-fluoro-N-methylaniline;
N-(4-cyanophenyl)-N-methyl-3-(5-methylpyridin-2-yl)imidazo[1,2-a]pyrazine-6-carboxamide;
N-(4-cyanophenyl)-3-(2,4-dichlorophenyl)-N-methylimidazo[1,2-a]pyrazine-6-carboxamide;
N-({3-[4-(difluoromethoxy)phenyl]imidazo[1,2-a]pyrazin-6-yl}methyl)-4-fluoro-N-methylaniline;
3-(4-carbamoylphenyl)-N-(3,4-difluorophenyl)-N-methylimidazo[1,2-a]pyrazine-6-carboxamide;
N-(2,4-difluorophenyl)-N-methyl-3-[4-(trifluoromethyl)phenyl]imidazo[1,2-a]pyrazine-6-carboxamide;
N-methyl-4-(morpholin-4-yl)-N-{3-[4-(trifluoromethyl)phenyl]imidazo[1,2-a]pyrazin-6-yl}benzamide;
4-[({3-[4-(5-amino-1,3,4-thiadiazol-2-yl)phenyl]imidazo[1,2-a]pyrazin-6-yl}methyl)(methyl)amino]benzonitrile;
3-(4-chloro-2-methylphenyl)-N-(4-cyanophenyl)-N-methylimidazo[1,2-a]pyrazine-6-carboxamide;
3-(2-chloropyridin-4-yl)-N-(4-cyanophenyl)-N-methylimidazo[1,2-a]pyrazine-6-carboxamide;
({3-[4-(5-amino-1,3,4-thiadiazol-2-yl)phenyl]imidazo[1,2-a]pyrazin-6-yl}methyl)-6-chloro-N-methylpyridin-3-amine;
N-(4-cyanophenyl)-3-(3,5-difluoropyridin-2-yl)-N-methylimidazo[1,2-a]pyrazine-6-carboxamide;
N-(4-cyanophenyl)-3-(4-methoxy-3-methylphenyl)-N-methylimidazo[1,2-a]pyrazine-6-carboxamide;
3-[2-chloro-4-(trifluoromethyl)phenyl]-N-(4-cyanophenyl)-N-methylimidazo[1,2-a]pyrazine-6-carboxamide;
N-(3-chloro-4-fluorophenyl)-N-methyl-3-[4-(trifluoromethyl)phenyl]imidazo[1,2-a]pyrazine-6-carboxamide;
3-acetyl-N-(6-chloropyridin-3-yl)-N-methylimidazo[1,2-a]pyrazine-6-carboxamide;
4-chloro-N-methyl-N-{[3-(1-methyl-1H-indazol-5-yl)imidazo[1,2-a]pyrazin-6-yl]methyl}aniline;
N-(3-chlorophenyl)-N-methyl-3-[4-(trifluoromethyl)phenyl]imidazo[1,2-a]pyrazine-6-carboxamide;
4-(6-{[(4-fluorophenyl)(methyl)amino]methyl}imidazo[1,2-a]pyrazin-3-yl)-N,N-dimethylaniline;
4-(6-{[(4-chlorophenyl)(methyl)amino]methyl}imidazo[1,2-a]pyrazin-3-yl)-N,N-dimethylaniline;
N-(4-cyanophenyl)-N-methyl-3-[4-(methylcarbamoyl)phenyl]imidazo[1,2-a]pyrazine-6-carboxamide;
N-(5-ethylpyridin-2-yl)-N-methyl-3-[4-(trifluoromethyl)phenyl]imidazo[1,2-a]pyrazine-6-carboxamide;
N-methyl-N-[2-(trifluoromethoxy)phenyl]-3-[4-(trifluoromethyl)phenyl]imidazo[1,2-a]pyrazine-6-carboxamide;
N-(2,4-dichlorophenyl)-3-(4-methoxyphenyl)-N-methylimidazo[1,2-a]pyrazine-6-carboxamide;
N-(3-chloro-4-methoxyphenyl)-N-(4-cyanophenyl)-N-methylimidazo[1,2-a]pyrazine-6-carboxamide;
N-methyl-N-(4-phenoxyphenyl)-3-[4-(trifluoromethyl)phenyl]imidazo[1,2-a]pyrazine-6-carboxamide;
4-chloro-N-{[3-(4-chlorophenyl)imidazo[1,2-a]pyrazin-6-yl]methyl}-N-methylaniline;
N,5-dimethyl-N-{[3-(1-methyl-1H-indazol-5-yl)imidazo[1,2-a]pyrazin-6-yl]methyl}pyridin-2-amine;
N-(4-cyanophenyl)-N-methyl-3-[3-(1H-pyrazol-4-yl)phenyl]imidazo[1,2-a]pyrazine-6-carboxamide;
N,5-dimethyl-N-({3-[4-(1H-pyrazol-5-yl)phenyl]imidazo[1,2-a]pyrazin-6-yl}methyl)pyridin-2-amine;
N-(4-cyanophenyl)-3-(3-methoxyphenyl)-N-methylimidazo[1,2-a]pyrazine-6-carboxamide;
N-(2,4-dichlorophenyl)-N-methyl-3-[4-(trifluoromethyl)phenyl]imidazo[1,2-a]pyrazine-6-carboxamide;
N-[4-(difluoromethoxy)phenyl]-N-methyl-3-[4-(trifluoromethyl)phenyl]imidazo[1,2-a]pyrazine-6-carboxamide;
N-methyl-N-[4-(1-methyl-1H-pyrazol-4-yl)phenyl]-3-[4-(trifluoromethyl)phenyl]imidazo[1,2-a]pyrazine-6-carboxamide;
N-(3-bromo-4-fluorophenyl)-N-methyl-3-[4-(trifluoromethyl)phenyl]imidazo[1,2-a]pyrazine-6-carboxamide;
N-(3,4-difluorophenyl)-N-methyl-3-(1-methyl-1H-indazol-5-yl)imidazo[1,2-a]pyrazine-6-carboxamide;
N-(2-fluoropyridin-3-yl)-N-methyl-3-[4-(trifluoromethyl)phenyl]imidazo[1,2-a]pyrazine-6-carboxamide;
N-(3,4-dichlorophenyl)-N-methyl-3-[4-(trifluoromethyl)phenyl]imidazo[1,2-a]pyrazine-6-carboxamide;
N-(4-chloro-3-methylphenyl)-N-methyl-3-[4-(trifluoromethyl)phenyl]imidazo[1,2-a]pyrazine-6-carboxamide;
N-(4-cyanophenyl)-3-[4-(difluoromethyl)phenyl]-N-methylimidazo[1,2-a]pyrazine-6-carboxamide;
N-(6-bromopyridin-3-yl)-N-methyl-3-[4-(trifluoromethyl)phenyl]imidazo[1,2-a]pyrazine-6-carboxamide;
N-(4-chloro-2-methoxyphenyl)-N-methyl-3-[4-(trifluoromethyl)phenyl]imidazo[1,2-a]pyrazine-6-carboxamide;
3-[4-chloro-3-(trifluoromethyl)phenyl]-N-(4-cyanophenyl)-N-methylimidazo[1,2-a]pyrazine-6-carboxamide;
N-(2,4-dichlorophenyl)-N-methyl-3-(4-methylphenyl)imidazo[1,2-a]pyrazine-6-carboxamide;
4-[methyl({[3-(1-methyl-1H-indazol-5-yl)imidazo[1,2-a]pyrazin-6-yl]methyl})amino]benzonitrile;
N-(5-fluoropyridin-2-yl)-N-methyl-3-[4-(trifluoromethyl)phenyl]imidazo[1,2-a]pyrazine-6-carboxamide;
4-chloro-N-({3-[4-(difluoromethoxy)phenyl]imidazo[1,2-a]pyrazin-6-yl}methyl)-N-methylaniline;
N-(4-cyanophenyl)-3-(2-fluoro-4-methoxyphenyl)-N-methylimidazo[1,2-a]pyrazine-6-carboxamide;
N-(5-fluoropyridin-3-yl)-N-methyl-3-[4-(trifluoromethyl)phenyl]imidazo[1,2-a]pyrazine-6-carboxamide;
N-(4-cyanophenyl)-3-(4-methoxyphenyl)-N-methylimidazo[1,2-a]pyrazine-6-carboxamide;
N-methyl-N-[4-(2-methyl-2H-1,2,3,4-tetrazol-5-yl)phenyl]-3-[4-(trifluoromethyl)phenyl]imidazo[1,2-a]pyrazine-6-carboxamide;
N-(4-fluorophenyl)-3-(4-methoxyphenyl)-N-methylimidazo[1,2-a]pyrazine-6-carboxamide;
3-(4-chlorophenyl)-N-(2,4-dichlorophenyl)-N-methylimidazo[1,2-a]pyrazine-6-carboxamide;
3-(4-chloro-3-fluorophenyl)-N-(4-cyanophenyl)-N-methylimidazo[1,2-a]pyrazine-6-carboxamide;
N-(4-cyanophenyl)-N-[2-(dimethylamino)ethyl]-3-[4-(trifluoromethyl)phenyl]imidazo[1,2-a]pyrazine-6-carboxamide;
N-(2-chlorophenyl)-N-methyl-3-[4-(trifluoromethyl)phenyl]imidazo[1,2-a]pyrazine-6-carboxamide;
3-[4-(5-amino-1,3,4-thiadiazol-2-yl)phenyl]-N-(4-cyanophenyl)-N-methylimidazo[1,2-a]pyrazine-6-carboxamide;
N-(4-cyanophenyl)-3-[4-(1,1-difluoroethyl)phenyl]-N-methylimidazo[1,2-a]pyrazine-6-carboxamide;
N-(4-cyanophenyl)-N-methyl-3-[4-(trifluoromethyl)phenyl]imidazo[1,2-a]pyrazine-6-carboxamide;
N-(4-cyanophenyl)-3-[4-(dimethylamino)phenyl]-N-methylimidazo[1,2-a]pyrazine-6-carboxamide;
N-(6-chloropyridin-3-yl)-N-methyl-3-[4-(trifluoromethyl)phenyl]imidazo[1,2-a]pyrazine-6-carboxamide;
N-(6-fluoropyridin-3-yl)-N-methyl-3-[4-(trifluoromethyl)phenyl]imidazo[1,2-a]pyrazine-6-carboxamide;
N-(6-chloropyridin-3-yl)-N-methyl-3-(4-methylphenyl)imidazo[1,2-a]pyrazine-6-carboxamide;
N-(4-chlorophenyl)-N-(6-chloropyridin-3-yl)-N-methylimidazo[1,2-a]pyrazine-6-carboxamide;
N-(4-cyanophenyl)-N-methyl-3-[6-(pyrrolidin-1-yl)pyridin-3-yl]imidazo[1,2-a]pyrazine-6-carboxamide;
N-(5-cyanopyridin-2-yl)-N-methyl-3-(1-methyl-1H-indazol-5-yl)imidazo[1,2-a]pyrazine-6-carboxamide;
N-(6-chloropyridin-3-yl)-N-methyl-3-(1-methyl-1H-indol-5-yl)imidazo[1,2-a]pyrazine-6-carboxamide;
N-(5-cyanopyridin-2-yl)-3-[4-(difluoromethoxy)phenyl]-N-methylimidazo[1,2-a]pyrazine-6-carboxamide;
N-(6-fluoropyridin-2-yl)-N-methyl-3-[4-(trifluoromethyl)phenyl]imidazo[1,2-a]pyrazine-6-carboxamide;
N-(4-cyanophenyl)-N-methyl-3-[4-(1H-pyrazol-1-yl)phenyl]imidazo[1,2-a]pyrazine-6-carboxamide;
N-(6-methoxypyridin-3-yl)-N-methyl-3-[4-(trifluoromethyl)phenyl]imidazo[1,2-a]pyrazine-6-carboxamide;
N-(5-cyanopyridin-2-yl)-N-methyl-3-[4-(trifluoromethyl)phenyl]imidazo[1,2-a]pyrazine-6-carboxamide;
N-(4-cyanophenyl)-3-(6-methoxypyridin-3-yl)-N-methylimidazo[1,2-a]pyrazine-6-carboxamide;
N-(5-chloropyrazin-2-yl)-N-methyl-3-[4-(trifluoromethyl)phenyl]imidazo[1,2-a]pyrazine-6-carboxamide;
N-(4-cyanophenyl)-N-(propan-2-yl)-3-[4-(trifluoromethyl)phenyl]imidazo[1,2-a]pyrazine-6-carboxamide;
N-(5-chloropyrimidin-2-yl)-N-methyl-3-[4-(trifluoromethyl)phenyl]imidazo[1,2-a]pyrazine-6-carboxamide;
N-(6-chloropyridazin-3-yl)-N-methyl-3-[4-(trifluoromethyl)phenyl]imidazo[1,2-a]pyrazine-6-carboxamide;
N-(5-cyanopyridin-2-yl)-3-[4-(difluoromethyl)phenyl]-N-methylimidazo[1,2-a]pyrazine-6-carboxamide;
N-(6-bromopyridazin-3-yl)-N-methyl-3-[4-(trifluoromethyl)phenyl]imidazo[1,2-a]pyrazine-6-carboxamide;
N-(5-bromopyrazin-2-yl)-N-methyl-3-[4-(trifluoromethyl)phenyl]imidazo[1,2-a]pyrazine-6-carboxamide;
3-(4-carbamoylphenyl)-N-(5-cyanopyridin-2-yl)-N-methylimidazo[1,2-a]pyrazine-6-carboxamide;
N-methyl-N-(pyridin-2-yl)-3-[4-(trifluoromethyl)phenyl]imidazo[1,2-a]pyrazine-6-carboxamide;
N-(6-chloropyridin-3-yl)-3-(4-methoxyphenyl)-N-methylimidazo[1,2-a]pyrazine-6-carboxamide;
N-(4-fluorophenyl)-N-methyl-3-[4-(trifluoromethyl)phenyl]imidazo[1,2-a]pyrazine-6-carboxamide;
N-(4-chlorophenyl)-3-[4-(difluoromethoxy)phenyl]-N-methylimidazo[1,2-a]pyrazine-6-carboxamide;
3-(4-chlorophenyl)-N-(4-fluorophenyl)-N-methylimidazo[1,2-a]pyrazine-6-carboxamide;
3-(4-carbamoylphenyl)-N-(4-cyanophenyl)-N-methylimidazo[1,2-a]pyrazine-6-carboxamide;
N-(3,4-difluorophenyl)-N-methyl-3-[4-(trifluoromethyl)phenyl]imidazo[1,2-a]pyrazine-6-carboxamide;
N-(4-fluorophenyl)-N-methyl-3-(1-methyl-1H-indazol-5-yl)imidazo[1,2-a]pyrazine-6-carboxamide;
N-(4-fluorophenyl)-N-methyl-3-(1-methyl-1H-indazol-5-yl)imidazo[1,2-a]pyrazine-6-carboxamide;
N-(4-fluorophenyl)-N-methyl-3-(4-methylphenyl)imidazo[1,2-a]pyrazine-6-carboxamide;
N,3-bis(4-chlorophenyl)-N-methylimidazo[1,2-a]pyrazine-6-carboxamide;
N-(2-methoxyphenyl)-N-methyl-3-[4-(trifluoromethyl)phenyl]imidazo[1,2-a]pyrazine-6-carboxamide;
6-fluoro-1-({3-[4-(trifluoromethyl)phenyl]imidazo[1,2-a]pyrazin-6-yl}carbonyl)-1,2,3,4-tetrahydroquinoline;
3-(4-chlorophenyl)-N-(3,4-difluorophenyl)-N-methylimidazo[1,2-a]pyrazine-6-carboxamide;
N-(4-cyanophenyl)-N-methyl-3-(4-methylphenyl)imidazo[1,2-a]pyrazine-6-carboxamide;
N-(4-cyanophenyl)-N-ethyl-3-[4-(trifluoromethyl)phenyl]imidazo[1,2-a]pyrazine-6-carboxamide;
N-(4-cyanophenyl)-N-methyl-3-[4-(propan-2-yl)phenyl]imidazo[1,2-a]pyrazine-6-carboxamide;
N-(4-cyanophenyl)-N-methyl-3-[4-(trifluoromethoxy)phenyl]imidazo[1,2-a]pyrazine-6-carboxamide;
N-(4-cyanophenyl)-N-methyl-3-[4-(methylamino)phenyl]imidazo[1,2-a]pyrazine-6-carboxamide;
3-(4-tert-butylphenyl)-N-(4-cyanophenyl)-N-methylimidazo[1,2-a]pyrazine-6-carboxamide;
N-(2-fluorophenyl)-N-methyl-3-[4-(trifluoromethyl)phenyl]imidazo[1,2-a]pyrazine-6-carboxamide;
N-[4-(1-cyano-1-methylethyl)phenyl]-N-(4-cyanophenyl)-N-methylimidazo[1,2-a]pyrazine-6-carboxamide;
3-[4-(dimethylamino)phenyl]-N-(4-fluorophenyl)-N-methylimidazo[1,2-a]pyrazine-6-carboxamide;
N-(4-chlorophenyl)-3-[4-(dimethylamino)phenyl]-N-methylimidazo[1,2-a]pyrazine-6-carboxamide;
N-(4-cyanophenyl)-N-methyl-3-(1-methyl-1H-indol-5-yl)imidazo[1,2-a]pyrazine-6-carboxamide;
3-(4-aminophenyl)-N-(4-cyanophenyl)-N-methylimidazo[1,2-a]pyrazine-6-carboxamide;
N-(4-cyanophenyl)-3-[4-(furan-2-yl)phenyl]-N-methylimidazo[1,2-a]pyrazine-6-carboxamide;
N-(4-cyano-3-fluorophenyl)-N-methyl-3-[4-(trifluoromethyl)phenyl]imidazo[1,2-a]pyrazine-6-carboxamide;
N-(3,4-difluorophenyl)-N-methyl-3-(1-methyl-1H-indol-5-yl)imidazo[1,2-a]pyrazine-6-carboxamide;
N-(4-methanesulfonylphenyl)-3-[4-(trifluoromethyl)phenyl]imidazo[1,2-a]pyrazine-6-carboxamide;
N-(4-cyanophenyl)-3-(4-methanesulfonylphenyl)-N-methylimidazo[1,2-a]pyrazine-6-carboxamide;
N-(4-cyanophenyl)-3-[4-(1-methoxyethyl)phenyl]-N-methylimidazo[1,2-a]pyrazine-6-carboxamide;
N-(4-methanesulfonylphenyl)-N-methyl-3-[4-(1H-pyrazol-5-yl)phenyl]imidazo[1,2-a]pyrazine-6-carboxamide;
3-(4-chlorophenyl)-N-(4-cyanophenyl)-N-methylimidazo[1,2-a]pyrazine-6-carboxamide;
N-(4-cyanophenyl)-N-[2-(oxan-4-yl)ethyl]-3-[4-(trifluoromethyl)phenyl]imidazo[1,2-a]pyrazine-6-carboxamide;
3-(4-acetylphenyl)-N-(4-cyanophenyl)-N-methylimidazo[1,2-a]pyrazine-6-carboxamide;
N-(3,4-difluorophenyl)-N-methyl-3-(4-methylphenyl)imidazo[1,2-a]pyrazine-6-carboxamide;
N-(4-fluorophenyl)-N-methyl-3-(1-methyl-1H-indol-5-yl)imidazo[1,2-a]pyrazine-6-carboxamide;
N-(3-fluorophenyl)-N-methyl-3-[4-(trifluoromethyl)phenyl]imidazo[1,2-a]pyrazine-6-carboxamide;
N-(4-cyanophenyl)-N-methyl-3-[4-(2,2,2-trifluoroethoxy)phenyl]imidazo[1,2-a]pyrazine-6-carboxamide;
N-(4-methoxyphenyl)-N-methyl-3-[4-(trifluoromethyl)phenyl]imidazo[1,2-a]pyrazine-6-carboxamide;
N-(4-cyanophenyl)-3-[2-fluoro-4-(trifluoromethyl)phenyl]-N-methylimidazo[1,2-a]pyrazine-6-carboxamide;
N-(4-chlorophenyl)-N-methyl-3-(1-methyl-1H-indol-5-yl)imidazo[1,2-a]pyrazine-6-carboxamide;
3-[4-(difluoromethoxy)phenyl]-N-(3,4-difluorophenyl)-N-methylimidazo[1,2-a]pyrazine-6-carboxamide;
3-(1H-1,3-benzodiazol-5-yl)-N-(4-cyanophenyl)-N-methylimidazo[1,2-a]pyrazine-6-carboxamide;
N-(3,4-difluorophenyl)-N-methyl-3-[4-(1H-pyrazol-1-yl)phenyl]imidazo[1,2-a]pyrazine-6-carboxamide;
N-(4-cyanophenyl)-3-(1H-indazol-5-yl)-N-methylimidazo[1,2-a]pyrazine-6-carboxamide;
N-(6-cyanopyridin-3-yl)-N-methyl-3-[4-(trifluoromethyl)phenyl]imidazo[1,2-a]pyrazine-6-carboxamide;
N-(6-cyanopyridin-3-yl)-N-methyl-3-[4-(trifluoromethyl)phenyl]imidazo[1,2-a]pyrazine-6-carboxamide;
N-(4-cyanophenyl)-N-methyl-3-(2-methyl-2H-indazol-5-yl)imidazo[1,2-a]pyrazine-6-carboxamide;
N-(4-cyanophenyl)-N-methyl-3-(2-methyl-2H-indazol-5-yl)imidazo[1,2-a]pyrazine-6-carboxamide;
N-(4-cyanophenyl)-N-methyl-3-[2-methyl-4-(trifluoromethyl)phenyl]imidazo[1,2-a]pyrazine-6-carboxamide;
N-(4-fluorophenyl)-N-methyl-3-[4-(1H-pyrazol-1-yl)phenyl]imidazo[1,2-a]pyrazine-6-carboxamide;
N-(4-cyanophenyl)-N-methyl-3-[4-(propane-2-sulfonyl)phenyl]imidazo[1,2-a]pyrazine-6-carboxamide;
N-(4-cyanophenyl)-3-(4-fluorophenyl)-N-methylimidazo[1,2-a]pyrazine-6-carboxamide;
N-(4-cyanophenyl)-3-(1H-indazol-6-yl)-N-methylimidazo[1,2-a]pyrazine-6-carboxamide;
3-(4-chloro-3-cyanophenyl)-N-(4-cyanophenyl)-N-methylimidazo[1,2-a]pyrazine-6-carboxamide;
N-(4-cyanophenyl)-N-methyl-3-[2-(propan-2-yl)-1,3-thiazol-4-yl]imidazo[1,2-a]pyrazine-6-carboxamide;
N-(4-cyanophenyl)-N-methyl-3-(quinolin-6-yl)imidazo[1,2-a]pyrazine-6-carboxamide;
N-(4-cyanophenyl)-3-[4-(cyclopropylcarbamoyl)phenyl]-N-methylimidazo[1,2-a]pyrazine-6-carboxamide;
N-(3,4-difluorophenyl)-3-[4-(dimethylamino)phenyl]-N-methylimidazo[1,2-a]pyrazine-6-carboxamide;
N-(4-cyanophenyl)-3-[4-(3,5-dimethyl-1H-pyrazol-1-yl)phenyl]-N-methylimidazo[1,2-a]pyrazine-6-carboxamide;
3-(4-carbamoylphenyl)-N-(6-chloropyridin-3-yl)-N-methylimidazo[1,2-a]pyrazine-6-carboxamide;
N-(4-cyanophenyl)-N-methyl-3-[4-(propan-2-yloxy)phenyl]imidazo[1,2-a]pyrazine-6-carboxamide;
N-(4-cyanophenyl)-N-methyl-3-[4-(5-methyl-1,3,4-oxadiazol-2-yl)phenyl]imidazo[1,2-a]pyrazine-6-carboxamide;
3-[4-(dimethylamino)phenyl]-N-(4-methanesulfonylphenyl)-N-methylimidazo[1,2-a]pyrazine-6-carboxamide;
N-(4-cyanophenyl)-3-[4-(2,5-dioxoimidazolidin-4-yl)phenyl]-N-methylimidazo[1,2-a]pyrazine-6-carboxamide;
methyl 4-{6-[(4-cyanophenyl)(methyl)carbamoyl]imidazo[1,2-a]pyrazin-3-yl}benzoate;
N-(4-cyanophenyl)-3-(isoquinolin-6-yl)-N-methylimidazo[1,2-a]pyrazine-6-carboxamide;
N-(4-cyanophenyl)-N-methyl-3-(1-oxo-1,2-dihydroisoquinolin-6-yl)imidazo[1,2-a]pyrazine-6-carboxamide;
N-(4-cyanophenyl)-3-[3-fluoro-4-(trifluoromethyl)phenyl]-N-methylimidazo[1,2-a]pyrazine-6-carboxamide;
N-(4-cyanophenyl)-N-methyl-3-(6-propoxypyridin-3-yl)imidazo[1,2-a]pyrazine-6-carboxamide;
N-methyl-N-[2-(trifluoromethyl)phenyl]-3-[4-(trifluoromethyl)phenyl]imidazo[1,2-a]pyrazine-6-carboxamide;
3-(4-carbamoylphenyl)-N-(4-methanesulfonylphenyl)-N-methylimidazo[1,2-a]pyrazine-6-carboxamide;
N-methyl-N-[4-(trifluoromethoxy)phenyl]-3-[4-(trifluoromethyl)phenyl]imidazo[1,2-a]pyrazine-6-carboxamide;
N-methyl-N-(1-methyl-1H-imidazol-2-yl)-3-[4-(trifluoromethyl)phenyl]imidazo[1,2-a]pyrazine-6-carboxamide;
N-(4-cyanophenyl)-N-methyl-3-[3-(trifluoromethyl)phenyl]imidazo[1,2-a]pyrazine-6-carboxamide;
5-fluoro-1-({3-[4-(trifluoromethyl)phenyl]imidazo[1,2-a]pyrazin-6-yl}carbonyl)-2,3-dihydro-1H-indole;
N-(4-cyanophenyl)-N-methyl-3-(4-phenoxyphenyl)imidazo[1,2-a]pyrazine-6-carboxamide;
3-(1-benzyl-1H-pyrazol-4-yl)-N-(4-cyanophenyl)-N-methylimidazo[1,2-a]pyrazine-6-carboxamide;
3-[4-chloro-2-(trifluoromethyl)phenyl]-N-(4-cyanophenyl)-N-methylimidazo[1,2-a]pyrazine-6-carboxamide;
N-(4-cyanophenyl)-N-methyl-3-[4-(1H-1,2,3,4-tetrazol-5-yl)phenyl]imidazo[1,2-a]pyrazine-6-carboxamide;
N-(4-cyanophenyl)-N-methyl-3-[4-(pyrrolidin-1-yl)phenyl]imidazo[1,2-a]pyrazine-6-carboxamide;
N-(5-cyanopyridin-2-yl)-N-methyl-3-[4-(1H-pyrazol-5-yl)phenyl]imidazo[1,2-a]pyrazine-6-carboxamide;
N-(3-bromophenyl)-N-methyl-3-[4-(trifluoromethyl)phenyl]imidazo[1,2-a]pyrazine-6-carboxamide;
N-(4-cyanophenyl)-N-methyl-3-(5-sulfamoylpyridin-3-yl)imidazo[1,2-a]pyrazine-6-carboxamide;
N-(4-cyanophenyl)-3-{4-[methoxy(methyl)carbamoyl]phenyl}-N-methylimidazo[1,2-a]pyrazine-6-carboxamide;
3-[4-(aminomethyl)phenyl]-N-(4-cyanophenyl)-N-methylimidazo[1,2-a]pyrazine-6-carboxamide;
N-(4-cyanophenyl)-N-methyl-3-[6-(trifluoromethyl)pyridin-3-yl]imidazo[1,2-a]pyrazine-6-carboxamide;
N-(4-cyanophenyl)-3-[4-(diethylcarbamoyl)phenyl]-N-methylimidazo[1,2-a]pyrazine-6-carboxamide;
N-(4-cyanophenyl)-N-methyl-3-(1-methyl-1H-pyrazol-4-yl)imidazo[1,2-a]pyrazine-6-carboxamide;
N-methyl-N-[(6-methylpyridin-2-yl)methyl]-3-[4-(trifluoromethyl)phenyl]imidazo[1,2-a]pyrazine-6-carboxamide;
3-(3-cyanophenyl)-N-(4-cyanophenyl)-N-methylimidazo[1,2-a]pyrazine-6-carboxamide;
N-[(4-fluorophenyl)methyl]-N-methyl-3-[4-(trifluoromethyl)phenyl]imidazo[1,2-a]pyrazine-6-carboxamide;
N-(4-cyanophenyl)-N-methyl-3-[4-(pyrrolidin-2-yl)phenyl]imidazo[1,2-a]pyrazine-6-carboxamide;
N-methyl-N-(pyridin-3-ylmethyl)-3-[4-(trifluoromethyl)phenyl]imidazo[1,2-a]pyrazine-6-carboxamide;
N-(4-cyanophenyl)-N-methyl-3-(pyridin-3-yl)imidazo[1,2-a]pyrazine-6-carboxamide;
3-(5-chloropyridin-2-yl)-N-(4-cyanophenyl)-N-methylimidazo[1,2-a]pyrazine-6-carboxamide;
N-(4-cyanophenyl)-3-[4-(dimethylsulfamoyl)phenyl]-N-methylimidazo[1,2-a]pyrazine-6-carboxamide;
N-(4-cyanophenyl)-N-methyl-3-[2-(4-methylpiperidin-1-yl)-1,3-thiazol-4-yl]imidazo[1,2-a]pyrazine-6-carboxamide;
N-(4-cyanophenyl)-N-methyl-3-(3-methylphenyl)imidazo[1,2-a]pyrazine-6-carboxamide;
N-(4-cyanophenyl)-N-methyl-3-(1H-pyrazol-4-yl)imidazo[1,2-a]pyrazine-6-carboxamide;
N-(4-cyanophenyl)-N-methyl-3-(1-methyl-2,3-dihydro-1H-indol-5-yl)imidazo[1,2-a]pyrazine-6-carboxamide;
3-(2,1,3-benzoxadiazol-5-yl)-N-(4-cyanophenyl)-N-methylimidazo[1,2-a]pyrazine-6-carboxamide;
N-(4-cyanophenyl)-N-methyl-3-{3-methyl-3H-imidazo[4,5-b]pyridin-6-yl}imidazo[1,2-a]pyrazine-6-carboxamide;
N-(4-cyanophenyl)-N-methyl-3-(2-oxo-2,3-dihydro-1H-indol-5-yl)imidazo[1,2-a]pyrazine-6-carboxamide;
N-(4-methanesulfonylphenyl)-N-methyl-3-(1-methyl-1H-indazol-5-yl)imidazo[1,2-a]pyrazine-6-carboxamide;
N-methyl-N-{4-[3-(trifluoromethyl)-1H-pyrazol-1-yl]phenyl}-3-[4-(trifluoromethyl)phenyl]imidazo[1,2-a]pyrazine-6-carboxamide;
N-(4-cyanophenyl)-3-{4-[(dimethylamino)methyl]phenyl}-N-methylimidazo[1,2-a]pyrazine-6-carboxamide;
N-(4-cyanophenyl)-N-methyl-3-[4-(1-methyl-1H-pyrazol-5-yl)phenyl]imidazo[1,2-a]pyrazine-6-carboxamide;
3-[4-(difluoromethoxy)phenyl]-N-(4-methanesulfonylphenyl)-N-methylimidazo[1,2-a]pyrazine-6-carboxamide;
4-[3-(4-carbamoylphenyl)imidazo[1,2-a]pyrazin-6-yl]benzamide;
N-(4-cyanophenyl)-N-methyl-3-[5-(2-oxopyrrolidin-1-yl)pyrazin-2-yl]imidazo[1,2-a]pyrazine-6-carboxamide;
3-{4-[(2S)-2-amino-2-cyclohexylacetamido]phenyl}-N-(4-cyanophenyl)-N-methylimidazo[1,2-a]pyrazine-6-carboxamide;
4-[methyl({3-[4-(trifluoromethyl)phenyl]imidazo[1,2-a]pyrazin-6-yl}methyl)amino]benzonitrile;
N-(4-cyanophenyl)-3-[4-(difluoromethoxy)phenyl]-N-methylimidazo[1,2-a]pyrazine-6-carboxamide;
N-(4-bromophenyl)-N-methyl-3-[4-(trifluoromethyl)phenyl]imidazo[1,2-a]pyrazine-6-carboxamide;
N-(6-chloropyridin-3-yl)-N-methyl-3-[4-(piperidin-1-yl)phenyl]imidazo[1,2-a]pyrazine-6-carboxamide;
N-(4-chlorophenyl)-N-methyl-3-[4-(trifluoromethyl)phenyl]imidazo[1,2-a]pyrazine-6-carboxamide;
6-chloro-1-({3-[4-(trifluoromethyl)phenyl]imidazo[1,2-a]pyrazin-6-yl}carbonyl)-1,2,3,4-tetrahydroquinoline;
N-(4-chlorophenyl)-3-(4-methoxyphenyl)-N-methylimidazo[1,2-a]pyrazine-6-carboxamide;
N-(2-chloro-5-methylpyridin-3-yl)-N-methyl-3-[4-(trifluoromethyl)phenyl]imidazo[1,2-a]pyrazine-6-carboxamide;
N-(3-fluoropyridin-2-yl)-N-methyl-3-[4-(trifluoromethyl)phenyl]imidazo[1,2-a]pyrazine-6-carboxamide;
4-(6-{[(4-fluorophenyl)(methyl)amino]methyl}imidazo[1,2-a]pyrazin-3-yl)benzamide;
4-(6-{[(4-cyanophenyl)(methyl)amino]methyl}imidazo[1,2-a]pyrazin-3-yl)benzamide;
N-(6-chloropyridin-3-yl)-N-methyl-3-[4-(1H-pyrazol-5-yl)phenyl]imidazo[1,2-a]pyrazine-6-carboxamide;
methyl N-[4-(6-{[(4-cyanophenyl)(methyl)amino]methyl}imidazo[1,2-a]pyrazin-3-yl)phenyl]carbamate
N-[5-(6-{[(4-cyanophenyl)(methyl)amino]methyl}imidazo[1,2-a]pyrazin-3-yl)pyridin-2-yl]acetamide;
3-[4-(5-amino-1,3,4-oxadiazol-2-yl)phenyl]-N-(4-fluorophenyl)-N-methylimidazo[1,2-a]pyrazine-6-carboxamide;
N-(4-chlorophenyl)-N-methyl-3-[4-(methylcarbamoyl)phenyl]imidazo[1,2-a]pyrazine-6-carboxamide;
4-{6-[(6-fluoro-2,2-dimethyl-1,2,3,4-tetrahydroquinolin-1-yl)carbonyl]imidazo[1,2-a]pyrazin-3-yl}benzamide;
6-fluoro-2,2-dimethyl-1-({3-[4-(trifluoromethyl)phenyl]imidazo[1,2-a]pyrazin-6-yl}carbonyl)-1,2,3,4-tetrahydroquinoline;
6-chloro-N-methyl-N-({3-[4-(trifluoromethyl)phenyl]imidazo[1,2-a]pyrazin-6-yl}methyl)pyridin-3-amine;
3-(4-cyanophenyl)-N-(4-methanesulfonylphenyl)-N-methylimidazo[1,2-a]pyrazine-6-carboxamide;
4-(6-{[methyl(5-methylpyridin-2-yl)amino]methyl}imidazo[1,2-a]pyrazin-3-yl)benzamide;
6-fluoro-1-({3-[4-(trifluoromethyl)phenyl]imidazo[1,2-a]pyrazin-6-yl}methyl)-1,2,3,4-tetrahydroquinoline;
N,5-dimethyl-N-({3-[4-(trifluoromethyl)phenyl]imidazo[1,2-a]pyrazin-6-yl}methyl)pyridin-3-amine;
N-(4-cyanophenyl)-3-(6-acetamidopyridin-3-yl)-N-methylimidazo[1,2-a]pyrazine-6-carboxamide;
N-(6-chloropyridin-3-yl)-3-[4-(1,1-difluoroethyl)phenyl]-N-methylimidazo[1,2-a]pyrazine-6-carboxamide;
N-(4-cyanophenyl)-N-(2-methoxyethyl)-3-[4-(trifluoromethyl)phenyl]imidazo[1,2-a]pyrazine-6-carboxamide;
N-methyl-3-[4-(trifluoromethyl)phenyl]-N-[6-(trifluoromethyl)pyridin-3-yl]imidazo[1,2-a]pyrazine-6-carboxamide;
N-(4-cyanophenyl)-N-methyl-3-[4-(1,3,4-oxadiazol-2-yl)phenyl]imidazo[1,2-a]pyrazine-6-carboxamide;
N-({3-[4-(difluoromethyl)phenyl]imidazo[1,2-a]pyrazin-6-yl}methyl)-N,5-dimethylpyridin-2-amine;
N-[5-(6-{[methyl(5-methylpyridin-2-yl)amino]methyl}imidazo[1,2-a]pyrazin-3-yl)pyridin-2-yl]acetamide;
N-(4-cyanophenyl)-N-methyl-3-{1H-pyrrolo[2,3-b]pyridin-5-yl}imidazo[1,2-a]pyrazine-6-carboxamide;
N-(4-fluorophenyl)-N-methyl-3-[4-(methylcarbamoyl)phenyl]imidazo[1,2-a]pyrazine-6-carboxamide;
methyl N-(4-{6-[(6-chloropyridin-3-yl)(methyl)carbamoyl]imidazo[1,2-a]pyrazin-3-yl}phenyl)carbamate;
N-(4-cyanophenyl)-N-methyl-3-[6-(methylamino)pyridin-3-yl]imidazo[1,2-a]pyrazine-6-carboxamide;
3-[4-(2-aminoacetamido)phenyl]-N-(4-cyanophenyl)-N-methylimidazo[1,2-a]pyrazine-6-carboxamide;
3-[4-(5-amino-1,3,4-oxadiazol-2-yl)phenyl]-N-(4-cyanophenyl)-N-methylimidazo[1,2-a]pyrazine-6-carboxamide;
N-methyl-N-(pyridin-4-yl)-3-[4-(trifluoromethyl)phenyl]imidazo[1,2-a]pyrazine-6-carboxamide;
N-(2-methoxy-5-methylpyridin-3-yl)-N-methyl-3-[4-(trifluoromethyl)phenyl]imidazo[1,2-a]pyrazine-6-carboxamide;
N-methyl-N-(5-methylpyridin-3-yl)-3-[4-(trifluoromethyl)phenyl]imidazo[1,2-a]pyrazine-6-carboxamide;
N-(2-methoxy-5-methylpyridin-3-yl)-N-methyl-3-[4-(trifluoromethyl)phenyl]imidazo[1,2-a]pyrazine-6-carboxamide;
ethyl 6-[(6-chloropyridin-3-yl)(methyl)carbamoyl]imidazo[1,2-a]pyrazine-3-carboxylate;
ethyl N-(4-{6-[(4-cyanophenyl)(methyl)carbamoyl]imidazo[1,2-a]pyrazin-3-yl}phenyl)carbamate;
3-(4-{[(1S,2R)-2-aminocyclopentane]amido}phenyl)-N-(4-cyanophenyl)-N-methylimidazo[1,2-a]pyrazine-6-carboxamide;
3-{4-[(3S)-3-amino-4-methylpentanamido]phenyl}-N-(4-cyanophenyl)-N-methylimidazo[1,2-a]pyrazine-6-carboxamide;
N-(4-fluorophenyl)-N-methyl-3-[4-(1H-pyrazol-5-yl)phenyl]imidazo[1,2-a]pyrazine-6-carboxamide;
N-(3,4-difluorophenyl)-N-methyl-3-[4-(1H-pyrazol-5-yl)phenyl]imidazo[1,2-a]pyrazine-6-carboxamide;
4-chloro-N-methyl-N-{3-[4-(trifluoromethyl)phenyl]imidazo[1,2-a]pyrazin-6-yl}benzamide;
methyl N-(5-{6-[(4-cyanophenyl)(methyl)carbamoyl]imidazo[1,2-a]pyrazin-3-yl}pyridin-2-yl)carbamate;
Propan-2-yl N-(4-{6-[(4-cyanophenyl)(methyl)carbamoyl]imidazo[1,2-a]pyrazin-3-yl}phenyl)carbamate;
N-(6-chloropyridin-3-yl)-N-methyl-3-{4-[5-(methylamino)-1,3,4-thiadiazol-2-yl]phenyl}imidazo[1,2-a]pyrazine-6-carboxamide;
N,4-dimethyl-N-({3-[4-(trifluoromethyl)phenyl]imidazo[1,2-a]pyrazin-6-yl}methyl)aniline;
5-chloro-N-methyl-N-{3-[4-(trifluoromethyl)phenyl]imidazo[1,2-a]pyrazin-6-yl}pyridine-2-carboxamide;
N-(6-chloropyridin-3-yl)-3-[4-(difluoromethyl)phenyl]-N-methylimidazo[1,2-a]pyrazine-6-carboxamide;
3-{4-[(2S)-2-amino-3-methylbutanamido]phenyl}-N-(4-cyanophenyl)-N-methylimidazo[1,2-a]pyrazine-6-carboxamide;
6-chloro-N-methyl-N-{3-[4-(trifluoromethyl)phenyl]imidazo[1,2-a]pyrazin-6-yl}pyridine-3-carboxamide;
4-fluoro-N-methyl-N-{3-[4-(trifluoromethyl)phenyl]imidazo[1,2-a]pyrazin-6-yl}benzamide;
3-{4-[(3R)-3-aminobutanamido]phenyl}-N-(4-cyanophenyl)-N-methylimidazo[1,2-a]pyrazine-6-carboxamide;
3-[4-(2-amino-2-cyclobutylacetamido)phenyl]-N-(4-cyanophenyl)-N-methylimidazo[1,2-a]pyrazine-6-carboxamide;
3-{4-[(2S)-2-aminopropanamido]phenyl}-N-(4-cyanophenyl)-N-methylimidazo[1,2-a]pyrazine-6-carboxamide;
3-(6-aminopyridin-3-yl)-N-(4-cyanophenyl)-N-methylimidazo[1,2-a]pyrazine-6-carboxamide;
3-[4-(cyanomethoxy)phenyl]-N-(4-cyanophenyl)-N-methylimidazo[1,2-a]pyrazine-6-carboxamide;
1-({3-[4-(trifluoromethyl)phenyl]imidazo[1,2-a]pyrazin-6-yl}carbonyl)-1,2,3,4-tetrahydroquinoline-6-carbonitrile;
N-(6-chloropyridin-3-yl)-3-[2-fluoro-4-(trifluoromethyl)phenyl]-N-methylimidazo[1,2-a]pyrazine-6-carboxamide;
N-methyl-4-(trifluoromethyl)-N-{3-[4-(trifluoromethyl)phenyl]imidazo[1,2-a]pyrazin-6-yl}benzamide;
N-methyl-N-(6-methylpyridin-3-yl)-3-[4-(trifluoromethyl)phenyl]imidazo[1,2-a]pyrazine-6-carboxamide;
3-{4-[(2-amino-2-methylpropyl)amino]phenyl}-N-(4-cyanophenyl)-N-methylimidazo[1,2-a]pyrazine-6-carboxamide;
N-(6-chloropyridin-3-yl)-3-(4-acetamidophenyl)-N-methylimidazo[1,2-a]pyrazine-6-carboxamide;
N-(6-chloropyridin-3-yl)-3-(2-fluoro-4-methoxyphenyl)-N-methylimidazo[1,2-a]pyrazine-6-carboxamide;
7-fluoro-4-({3-[4-(trifluoromethyl)phenyl]imidazo[1,2-a]pyrazin-6-yl}methyl)-3,4-dihydro-2H-1,4-benzoxazine;
3-{4-[(2R)-2-aminopropanamido]phenyl}-N-(4-cyanophenyl)-N-methylimidazo[1,2-a]pyrazine-6-carboxamide;
4-cyano-N-methyl-N-{3-[4-(trifluoromethyl)phenyl]imidazo[1,2-a]pyrazin-6-yl}benzamide;
N-(4-cyanophenyl)-3-{4-[2-(dimethylamino)ethoxy]phenyl}-N-methylimidazo[1,2-a]pyrazine-6-carboxamide;
N-(4-cyanophenyl)-N,2-dimethyl-3-[4-(trifluoromethyl)phenyl]imidazo[1,2-a]pyrazine-6-carboxamide;
3-{4-[(2R)-2-amino-3-methylbutanamido]phenyl}-N-(4-cyanophenyl)-N-methylimidazo[1,2-a]pyrazine-6-carboxamide;
N,5-dimethyl-N-({3-[4-(trifluoromethyl)phenyl]imidazo[1,2-a]pyrazin-6-yl}methyl)pyridin-2-amine;
N-(6-methoxy-5-methylpyridin-3-yl)-N-methyl-3-[4-(trifluoromethyl)phenyl]imidazo[1,2-a]pyrazine-6-carboxamide;
N,4-dimethyl-N-{3-[4-(trifluoromethyl)phenyl]imidazo[1,2-a]pyrazin-6-yl}benzamide;
N-(6-methoxypyridin-2-yl)-N-methyl-3-[4-(trifluoromethyl)phenyl]imidazo[1,2-a]pyrazine-6-carboxamide;
N-(4-cyanophenyl)-3-(4-{[2-(dimethylamino)ethyl]carbamoyl}phenyl)-N-methylimidazo[1,2-a]pyrazine-6-carboxamide;
3-{4-[(2S)-2-amino-2-cyclopropylacetamido]phenyl}-N-(4-cyanophenyl)-N-methylimidazo[1,2-a]pyrazine-6-carboxamide;
N-(4-cyano-2-fluorophenyl)-N-methyl-3-[4-(trifluoromethyl)phenyl]imidazo[1,2-a]pyrazine-6-carboxamide;
N,4,4-trimethyl-N-{3-[4-(trifluoromethyl)phenyl]imidazo[1,2-a]pyrazin-6-yl}piperidine-1-carboxamide;
3-[6-(2-aminoacetamido)pyridin-3-yl]-N-(4-cyanophenyl)-N-methylimidazo[1,2-a]pyrazine-6-carboxamide;
N-(2-methoxypyridin-4-yl)-N-methyl-3-[4-(trifluoromethyl)phenyl]imidazo[1,2-a]pyrazine-6-carboxamide;
N-(4-cyanophenyl)-N-methyl-3-(6-methylpyridin-3-yl)imidazo[1,2-a]pyrazine-6-carboxamide;
N-(1-ethyl-1H-pyrazol-5-yl)-N-methyl-3-[4-(trifluoromethyl)phenyl]imidazo[1,2-a]pyrazine-6-carboxamide;
N-methyl-N-(2-methylpyridin-4-yl)-3-[4-(trifluoromethyl)phenyl]imidazo[1,2-a]pyrazine-6-carboxamide;
tert-butyl N-{2-[(4-{6-[(4-cyanophenyl)(methyl)carbamoyl]imidazo[1,2-a]pyrazin-3-yl}phenyl)amino]-2-methylpropyl}carbamate;
N-(6-chloropyridin-3-yl)-3-(1H-indol-5-yl)-N-methylimidazo[1,2-a]pyrazine-6-carboxamide;
3-(4-methoxy-2-methylphenyl)-N-[6-(4-methoxy-2-methylphenyl)pyridin-3-yl]-N-methylimidazo[1,2-a]pyrazine-6-carboxamide;
N-(4-methoxypyridin-3-yl)-N-methyl-3-[4-(trifluoromethyl)phenyl]imidazo[1,2-a]pyrazine-6-carboxamide;
N-(6-chloro-5-methylpyridin-3-yl)-N-methyl-3-[4-(trifluoromethyl)phenyl]imidazo[1,2-a]pyrazine-6-carboxamide;
7-fluoro-4-({3-[4-(trifluoromethyl)phenyl]imidazo[1,2-a]pyrazin-6-yl}carbonyl)-3,4-dihydro-2H-1,4-benzoxazine;
({3-[4-(trifluoromethyl)phenyl]imidazo[1,2-a]pyrazin-6-yl}carbonyl)-3,4-dihydro-2H-1,4-benzoxazine;
3-(4-{[1-(aminomethyl)cyclopropane]amido}phenyl)-N-(4-cyanophenyl)-N-methylimidazo[1,2-a]pyrazine-6-carboxamide;
N-methyl-6-(trifluoromethyl)-N-{3-[4-(trifluoromethyl)phenyl]imidazo[1,2-a]pyrazin-6-yl}pyridine-3-carboxamide;
N-methyl-N-(4-methylpyridin-3-yl)-3-[4-(trifluoromethyl)phenyl]imidazo[1,2-a]pyrazine-6-carboxamide;
N-(4-ethoxypyridin-2-yl)-N-methyl-3-[4-(trifluoromethyl)phenyl]imidazo[1,2-a]pyrazine-6-carboxamide;
4-methoxy-N-methyl-N-{3-[4-(trifluoromethyl)phenyl]imidazo[1,2-a]pyrazin-6-yl}benzamide;
N-(6-chloropyridin-3-yl)-N-methyl-3-{1H-pyrrolo[2,3-b]pyridin-5-yl}imidazo[1,2-a]pyrazine-6-carboxamide;
N-methyl-N-[4-(1H-1,2,4-triazol-1-yl)phenyl]-3-[4-(trifluoromethyl)phenyl]imidazo[1,2-a]pyrazine-6-carboxamide;
N-(4-cyanophenyl)-3-(4-methanesulfonamidophenyl)-N-methylimidazo[1,2-a]pyrazine-6-carboxamide;
4-({3-[4-(trifluoromethyl)phenyl]imidazo[1,2-a]pyrazin-6-yl}carbonyl)-3,4-dihydro-2H-1,4-benzoxazine-7-carbonitrile;
6-methanesulfonyl-1-({3-[4-(trifluoromethyl)phenyl]imidazo[1,2-a]pyrazin-6-yl}carbonyl)-1,2,3,4-tetrahydroquinoline;
N-(5-methanesulfonylpyridin-2-yl)-N-methyl-3-[4-(trifluoromethyl)phenyl]imidazo[1,2-a]pyrazine-6-carboxamide;
methyl N-[4-(6-{[methyl(5-methylpyridin-2-yl)amino]methyl}imidazo[1,2-a]pyrazin-3-yl)phenyl]carbamate;
3-(1H-indol-2-yl)-N-[6-(1H-indol-2-yl)pyridin-3-yl]-N-methylimidazo[1,2-a]pyrazine-6-carboxamide;
N-(6-chloropyridin-3-yl)-N-methyl-3-(3-methyl-1H-indazol-5-yl)imidazo[1,2-a]pyrazine-6-carboxamide;
N-methyl-N-[5-(morpholin-4-yl)pyridin-2-yl]-3-[4-(trifluoromethyl)phenyl]imidazo[1,2-a]pyrazine-6-carboxamide;
4-fluoro-N-methyl-N-{3-[4-(trifluoromethyl)phenyl]imidazo[1,2-a]pyrazin-6-yl}piperidine-1-carboxamide;
N-(4-cyanophenyl)-3-(6-methanesulfonamidopyridin-3-yl)-N-methylimidazo[1,2-a]pyrazine-6-carboxamide;
3-{4-[(1-amino-2-methylpropan-2-yl)amino]phenyl}-N-(4-cyanophenyl)-N-methylimidazo[1,2-a]pyrazine-6-carboxamide;
3-(1-benzofuran-5-yl)-N-(6-chloropyridin-3-yl)-N-methylimidazo[1,2-a]pyrazine-6-carboxamide;
N-methyl-N-(pyridin-3-yl)-3-[4-(trifluoromethyl)phenyl]imidazo[1,2-a]pyrazine-6-carboxamide;
N-methyl-N-(5-methylpyrazin-2-yl)-3-[4-(trifluoromethyl)phenyl]imidazo[1,2-a]pyrazine-6-carboxamide;
N-methyl-N-(5-methylpyrazin-2-yl)-3-[4-(trifluoromethyl)phenyl]imidazo[1,2-a]pyrazine-6-carboxamide;
N-methyl-3-[4-(trifluoromethyl)phenyl]-N-[2-(trifluoromethyl)pyridin-4-yl]imidazo[1,2-a]pyrazine-6-carboxamide;
N-(6-chloropyridin-3-yl)-N-methyl-3-[4-(1H-pyrazol-1-yl)phenyl]imidazo[1,2-a]pyrazine-6-carboxamide;
5-{6-[(4-cyanophenyl)(methyl)carbamoyl]imidazo[1,2-a]pyrazin-3-yl}-N-methylpyridine-2-carboxamide;
N-(6-chloropyridin-3-yl)-3-[3-fluoro-4-(trifluoromethyl)phenyl]-N-methylimidazo[1,2-a]pyrazine-6-carboxamide;
N-(4-cyanophenyl)-3-(2-methoxypyrimidin-5-yl)-N-methylimidazo[1,2-a]pyrazine-6-carboxamide;
N-(4-ethylpyridin-2-yl)-3-[4-(trifluoromethyl)phenyl]imidazo[1,2-a]pyrazine-6-carboxamide;
N-(4-cyanophenyl)-3-(6-methoxypyrazin-2-yl)-N-methylimidazo[1,2-a]pyrazine-6-carboxamide;
N-(6-chloropyridin-3-yl)-3-(6-methoxypyridin-3-yl)-N-methylimidazo[1,2-a]pyrazine-6-carboxamide;
N-methyl-3-[4-(trifluoromethyl)phenyl]-N-[5-(trifluoromethyl)pyridin-2-yl]imidazo[1,2-a]pyrazine-6-carboxamide;
N-methyl-3-[4-(trifluoromethyl)phenyl]-N-[5-(trifluoromethyl)pyridin-2-yl]imidazo[1,2-a]pyrazine-6-carboxamide;
N-(4-cyanophenyl)-N-methyl-3-[4-(3-methyl-1,2,4-oxadiazol-5-yl)phenyl]imidazo[1,2-a]pyrazine-6-carboxamide;
N-(6-chloropyridin-3-yl)-N-methyl-3-[4-(morpholin-4-yl)phenyl]imidazo[1,2-a]pyrazine-6-carboxamide;
N,3-bis(4-cyanophenyl)-N-methyl-2-(trifluoromethyl)imidazo[1,2-a]pyrazine-6-carboxamide;
N-(2,6-dichloropyridin-3-yl)-N-methyl-3-[4-(trifluoromethyl)phenyl]imidazo[1,2-a]pyrazine-6-carboxamide;
N-(6-chloropyridin-3-yl)-3-[4-(cyclopropylcarbamoyl)phenyl]-N-methylimidazo[1,2-a]pyrazine-6-carboxamide;
N-(4-cyanophenyl)-3-[2-(dimethylamino)pyrimidin-5-yl]-N-methylimidazo[1,2-a]pyrazine-6-carboxamide;
N-methyl-N-(6-methylpyridin-2-yl)-3-[4-(trifluoromethyl)phenyl]imidazo[1,2-a]pyrazine-6-carboxamide;
N-[2-(difluoromethoxy)phenyl]-N-methyl-3-[4-(trifluoromethyl)phenyl]imidazo[1,2-a]pyrazine-6-carboxamide;
4-cyano-N-methyl-N-{3-[4-(trifluoromethyl)phenyl]imidazo[1,2-a]pyrazin-6-yl}benzene-1-sulfonamide;
N-methyl-3-[4-(trifluoromethyl)phenyl]-N-[4-(trifluoromethyl)pyridin-2-yl]imidazo[1,2-a]pyrazine-6-carboxamide;
N-(6-chloropyridin-3-yl)-3-(1H-indazol-5-yl)-N-methylimidazo[1,2-a]pyrazine-6-carboxamide;
2,2-dimethyl-4-({3-[4-(trifluoromethyl)phenyl]imidazo[1,2-a]pyrazin-6-yl}carbonyl)-3,4-dihydro-2H-1,4-benzoxazine-7-carbonitrile;
N-(4-cyanophenyl)-3-(6-fluoropyridin-2-yl)-N-methylimidazo[1,2-a]pyrazine-6-carboxamide;
3-[4-(1-carbamoyl-1-methylethoxy)phenyl]-N-(4-cyanophenyl)-N-methylimidazo[1,2-a]pyrazine-6-carboxamide;
N-(5-methoxypyridin-2-yl)-N-methyl-3-[4-(trifluoromethyl)phenyl]imidazo[1,2-a]pyrazine-6-carboxamide;
3-(3-chloropyridin-2-yl)-N-(4-cyanophenyl)-N-methylimidazo[1,2-a]pyrazine-6-carboxamide;
N-(5-chloropyridin-3-yl)-N-methyl-3-[4-(trifluoromethyl)phenyl]imidazo[1,2-a]pyrazine-6-carboxamide;
N-methyl-N-(1,3-thiazol-2-yl)-3-[4-(trifluoromethyl)phenyl]imidazo[1,2-a]pyrazine-6-carboxamide;
N-(6-chloropyridin-3-yl)-3-[6-(dimethylamino)pyridin-3-yl]-N-methylimidazo[1,2-a]pyrazine-6-carboxamide;
N-(4-methoxypyridin-2-yl)-N-methyl-3-[4-(trifluoromethyl)phenyl]imidazo[1,2-a]pyrazine-6-carboxamide;
N-{6-[(2S)-2-(methoxymethyl)pyrrolidin-1-yl]pyridin-3-yl}-N-methyl-3-[4-(trifluoromethyl)phenyl]imidazo[1,2-a]pyrazine-6-carboxamide;
N-(6-chloropyridin-3-yl)-3-(2,3-dihydro-1,4-benzodioxin-6-yl)-N-methylimidazo[1,2-a]pyrazine-6-carboxamide;
3-(4-acetamidophenyl)-N-methyl-N-(5-methylpyridin-2-yl)imidazo[1,2-a]pyrazine-6-carboxamide;
N-[(4-cyanophenyl)methyl]-3-[4-(trifluoromethyl)phenyl]imidazo[1,2-a]pyrazine-6-carboxamide;
N-(4-cyanophenyl)-N-methyl-3-[4-(propane-2-sulfonamido)phenyl]imidazo[1,2-a]pyrazine-6-carboxamide;
N-(6-chloropyridin-3-yl)-N-methyl-3-[2-(propan-2-yl)-1,3-thiazol-4-yl]imidazo[1,2-a]pyrazine-6-carboxamide;
N-methyl-N-[6-(1H-1,2,4-triazol-1-yl)pyridin-3-yl]-3-[4-(trifluoromethyl)phenyl]imidazo[1,2-a]pyrazine-6-carboxamide;
N-methyl-4-(trifluoromethoxy)-N-{3-[4-(trifluoromethyl)phenyl]imidazo[1,2-a]pyrazin-6-yl}benzamide;
N-(6-chloropyridin-3-yl)-N-methyl-3-[6-(5-methyl-1,3,4-oxadiazol-2-yl)pyridin-3-yl]imidazo[1,2-a]pyrazine-6-carboxamide;
N-(1-ethyl-1H-pyrazol-5-yl)-3-[4-(trifluoromethyl)phenyl]imidazo[1,2-a]pyrazine-6-carboxamide;
3-(4-cyanophenyl)-N-methyl-N-[4-(trifluoromethyl)phenyl]imidazo[1,2-a]pyrazine-6-carboxamide;
N-benzyl-N-methyl-3-[4-(trifluoromethyl)phenyl]imidazo[1,2-a]pyrazine-6-carboxamide;
2-C-3-[4-(trifluoromethyl)phenyl]imidazo[1,2-a]pyrazine-6-pyridine-2,5-dicarboxamide;
3-[4-(2-aminoethoxy)phenyl]-N-(4-cyanophenyl)-N-methylimidazo[1,2-a]pyrazine-6-carboxamide;
3-(6-acetamidopyridin-3-yl)-N-methyl-N-(5-methylpyridin-2-yl)imidazo[1,2-a]pyrazine-6-carboxamide;
4-({3-[4-(trifluoromethyl)phenyl]imidazo[1,2-a]pyrazin-6-yl}carbonyl)morpholine;
N-[4-(aminomethyl)phenyl]-N-methyl-3-[4-(trifluoromethyl)phenyl]imidazo[1,2-a]pyrazine-6-carboxamide;
N-(4-cyanophenyl)-2-methyl-3-[4-(trifluoromethyl)phenyl]imidazo[1,2-a]pyrazine-6-carboxamide;
N,3-bis(4-cyanophenyl)-N-methylimidazo[1,2-a]pyrazine-6-carboxamide;
N-(4-cyanophenyl)-N-methyl-3-(2-methylpyridin-4-yl)imidazo[1,2-a]pyrazine-6-carboxamide;
N-(6-chloropyridin-3-yl)-N-methyl-3-(2-oxo-2,3-dihydro-1H-indol-5-yl)imidazo[1,2-a]pyrazine-6-carboxamide;
3-(2-aminopyrimidin-5-yl)-N-(4-cyanophenyl)-N-methylimidazo[1,2-a]pyrazine-6-carboxamide;
N-(6-chloropyridin-3-yl)-3-ethenyl-N-methylimidazo[1,2-a]pyrazine-6-carboxamide;
N-(5-cyclobutylpyridin-2-yl)-N-methyl-3-[4-(trifluoromethyl)phenyl]imidazo[1,2-a]pyrazine-6-carboxamide;
N-{[4-(morpholin-4-yl)phenyl]methyl}-3-[4-(trifluoromethyl)phenyl]imidazo[1,2-a]pyrazine-6-carboxamide;
N-(6-tert-butylpyridin-3-yl)-N-methyl-3-[4-(trifluoromethyl)phenyl]imidazo[1,2-a]pyrazine-6-carboxamide;
N-(4-cyanophenyl)-N-methyl-3-(pyridin-4-yl)imidazo[1,2-a]pyrazine-6-carboxamide;
N-(2-aminoethyl)-N-(4-cyanophenyl)-3-[4-(trifluoromethyl)phenyl]imidazo[1,2-a]pyrazine-6-carboxamide;
N-(6-chloropyridin-3-yl)-3-(1H-indol-2-yl)-N-methylimidazo[1,2-a]pyrazine-6-carboxamide;
N-(6-chloropyridin-3-yl)-3-{4-[(dimethylamino)methyl]phenyl}-N-methylimidazo[1,2-a]pyrazine-6-carboxamide;
N-(4-cyano-1H-imidazol-5-yl)-3-[4-(trifluoromethyl)phenyl]imidazo[1,2-a]pyrazine-6-carboxamide;
N-methyl-N-(3-methylpyridin-2-yl)-3-[4-(trifluoromethyl)phenyl]imidazo[1,2-a]pyrazine-6-carboxamide;
N-(4-cyanophenyl)-N-methyl-3-{1-methyl-1H-pyrazolo[3,4-b]pyridin-5-yl}imidazo[1,2-a]pyrazine-6-carboxamide;
4-({3-[4-(trifluoromethyl)phenyl]imidazo[1,2-a]pyrazin-6-yl}methyl) morpholine;
5-{6-[(4-cyanophenyl)(methyl)carbamoyl]imidazo[1,2-a]pyrazin-3-yl}pyridine-2-carboxamide;
N-[6-(dimethylamino)pyridin-3-yl]-N-methyl-3-[4-(trifluoromethyl)phenyl]imidazo[1,2-a]pyrazine-6-carboxamide;
N-(6-methoxypyridazin-3-yl)-N-methyl-3-[4-(trifluoromethyl)phenyl]imidazo[1,2-a]pyrazine-6-carboxamide;
3-(3-amino-1H-indazol-5-yl)-N-(4-cyanophenyl)-N-methylimidazo[1,2-a]pyrazine-6-carboxamide;
1-({3-[4-(trifluoromethyl)phenyl]imidazo[1,2-a]pyrazin-6-yl}carbonyl)piperidine-3-carboxamide;
N-(6-chloropyridin-3-yl)-N-methyl-3-[4-(methylamino)phenyl]imidazo[1,2-a]pyrazine-6-carboxamide;
N-(2-methoxypyrimidin-5-yl)-N-methyl-3-[4-(trifluoromethyl)phenyl]imidazo[1,2-a]pyrazine-6-carboxamide;
N-(4-cyanophenyl)-N-methyl-3-(6-{[2-(morpholin-4-yl)ethyl]amino}pyridin-3-yl)imidazo[1,2-a]pyrazine-6-carboxamide;
N-(4-cyanophenyl)-3-(6-cyanopyridin-3-yl)-N-methylimidazo[1,2-a]pyrazine-6-carboxamide;
3-{4-[(1-amino-2-methylpropan-2-yl)oxy]phenyl}-N-(4-cyanophenyl)-N-methylimidazo[1,2-a]pyrazine-6-carboxamide;
6-N-(6-chloropyridin-3-yl)-6-N-methyl-3-N-[4-(trifluoromethyl)phenyl]imidazo[1,2-a]pyrazine-3,6-dicarboxamide;
5-C-3-[4-(trifluoromethyl)phenyl]imidazo[1,2-a]pyrazine-6-1H-imidazole-4,5-dicarboxamide;
N-[1-(4-chlorophenyl)ethyl]-N-methyl-3-[4-(trifluoromethyl)phenyl]imidazo[1,2-a]pyrazine-6-carboxamide;
N-{4-[(1,3-thiazol-2-yl)sulfamoyl]phenyl}-3-[4-(trifluoromethyl)phenyl]imidazo[1,2-a]pyrazine-6-carboxamide;
N-(6-chloropyridin-3-yl)-N-methyl-3-[6-(methylamino)pyridin-3-yl]imidazo[1,2-a]pyrazine-6-carboxamide;
3-[4-(2-amino-2-methylpropanamido)phenyl]-N-(4-cyanophenyl)-N-methylimidazo[1,2-a]pyrazine-6-carboxamide;
3-[4-(2-aminoethoxy)phenyl]-N-methyl-N-(5-methylpyridin-2-yl)imidazo[1,2-a]pyrazine-6-carboxamide;
N-(6-chloropyridin-3-yl)-N-methyl-3-[4-(1,3,4-oxadiazol-2-yl)phenyl]imidazo[1,2-a]pyrazine-6-carboxamide;
6-fluoro-1-({8-methyl-3-[4-(trifluoromethyl)phenyl]imidazo[1,2-a]pyrazin-6-yl}carbonyl)-1,2,3,4-tetrahydroquinoline;
N-(5-cyclopentylpyridin-2-yl)-N-methyl-3-[4-(trifluoromethyl)phenyl]imidazo[1,2-a]pyrazine-6-carboxamide;
N-(6-chloropyridin-3-yl)-N-methyl-3-[6-(trifluoromethyl)pyridin-3-yl]imidazo[1,2-a]pyrazine-6-carboxamide;
N-[2-(4-fluorophenyl)propan-2-yl]-3-[4-(trifluoromethyl)phenyl]imidazo[1,2-a]pyrazine-6-carboxamide;
methyl N-(5-{6-[methyl(5-methylpyridin-2-yl)carbamoyl]imidazo[1,2-a]pyrazin-3-yl}pyridin-2-yl)carbamate;
N-methyl-N-{[1,2,4]triazolo[4,3-a]pyridin-5-yl}-3-[4-(trifluoromethyl)phenyl]imidazo[1,2-a]pyrazine-6-carboxamide;
4-tert-butyl-N-methyl-N-{3-[4-(trifluoromethyl)phenyl]imidazo[1,2-a]pyrazin-6-yl}benzamide;
N-(6-chloropyridin-3-yl)-3-[3-(dimethylamino)phenyl]-N-methylimidazo[1,2-a]pyrazine-6-carboxamide;
N-(6-chloropyridin-3-yl)-3-(4-methanesulfonamidophenyl)-N-methylimidazo[1,2-a]pyrazine-6-carboxamide;
N-methyl-N-(2-methylpyridin-3-yl)-3-[4-(trifluoromethyl)phenyl]imidazo[1,2-a]pyrazine-6-carboxamide;
N-methyl-N-(5-methylpyrimidin-2-yl)-3-[4-(trifluoromethyl)phenyl]imidazo[1,2-a]pyrazine-6-carboxamide;
N-(6-chloropyridin-3-yl)-3-(4-fluorophenyl)-N-methylimidazo[1,2-a]pyrazine-6-carboxamide;
N-methyl-N-(4-phenyl-1,3-thiazol-2-yl)-3-[4-(trifluoromethyl)phenyl]imidazo[1,2-a]pyrazine-6-carboxamide;
cyanophenyl)-N-methyl-3-(3-methyl-1H-indazol-5-yl)imidazo[1,2-a]pyrazine-6-carboxamide;
N-(4-acetylphenyl)-N-methyl-3-[4-(trifluoromethyl)phenyl]imidazo[1,2-a]pyrazine-6-carboxamide;
1-({3-[4-(trifluoromethyl)phenyl]imidazo[1,2-a]pyrazin-6-yl}methyl)piperidin-2-one;
N-(4-cyanophenyl)-3-[4-(dimethylcarbamoyl)phenyl]-N-methylimidazo[1,2-a]pyrazine-6-carboxamide;
N-(4-cyanophenyl)-3-(1H-indol-5-yl)-N-methylimidazo[1,2-a]pyrazine-6-carboxamide;
N-(6-chloropyridin-3-yl)-3-[4-(difluoromethoxy)phenyl]-N-methylimidazo[1,2-a]pyrazine-6-carboxamide;
N-(4-fluoro-2-methoxyphenyl)-N-methyl-3-[4 (trifluoromethyl)phenyl]imidazo[1,2-a]pyrazine;
N-(3,4-difluorophenyl)-3-(4-methoxyphenyl)-N-methylimidazo[1,2-a]pyrazine-6-carboxamide;
N-(4-chlorophenyl)-N-methyl-3-(4-methylphenyl)imidazo[1,2-a]pyrazine-6-carboxamide;
3-[4-(difluoromethoxy)phenyl]-N-(4-fluorophenyl)-N-methylimidazo[1,2-a]pyrazine-6-carboxamide;
N-(5-bromopyrimidin-2-yl)-N-methyl-3-[4-(trifluoromethyl)phenyl]imidazo[1,2-a]pyrazine-6-carboxamide;
N-(5-chloropyridin-2-yl)-N-methyl-3-[4-(trifluoromethyl)phenyl]imidazo[1,2-a]pyrazine-6-carboxamide;
N-methyl-N-[4-(propan-2-yloxy)phenyl]-3-[4-(trifluoromethyl)phenyl]imidazo[1,2-a]pyrazine-6-carboxamide;
4-[({3-[4-(dimethylamino)phenyl]imidazo[1,2-a]pyrazin-6-yl}methyl)(methyl)amino]benzonitrile;
N-(4-cyanophenyl)-3-[3-(dimethylamino)phenyl]-N-methylimidazo[1,2-a]pyrazine-6-carboxamide;
3-(6-chloropyridin-3-yl)-N-(4-cyanophenyl)-N-methylimidazo[1,2-a]pyrazine-6-carboxamide;
N-(3-methoxyphenyl)-N-methyl-3-[4-(trifluoromethyl)phenyl]imidazo[1,2-a]pyrazine-6-carboxamide;
N-(4-cyanophenyl)-3-(4-methoxy-2-methylphenyl)-N-methylimidazo[1,2-a]pyrazine-6-carboxamide;
N-(7-chloro-1,3-benzothiazol-2-yl)-3-[4-(trifluoromethyl)phenyl]imidazo[1,2-a]pyrazine-6-carboxamide;
4-fluoro-N-methyl-N-(3-{4-[5-(methylamino)-1,3,4-thiadiazol-2-yl]phenyl}imidazo[1,2-a]pyrazin-6-yl)benzamide;
3-{6-[(6-fluoro-1,2,3,4-tetrahydroquinolin-1-yl)carbonyl]imidazo[1,2-a]pyrazin-3-yl}-N,N-dimethylaniline;
N,3-bis[4-(trifluoromethyl)phenyl]imidazo[1,2-a]pyrazine-6-carboxamide;
N-(4-cyanophenyl)-N-methyl-3-{4-[5-(methylamino)-1,3,4-oxadiazol-2-yl]phenyl}imidazo[1,2-a]pyrazine-6-carboxamide;
4-{6-[(7-fluoro-3,3-dimethyl-3,4-dihydro-2H-1,4-benzoxazin-4-yl)carbonyl]imidazo[1,2-a]pyrazin-3-yl}benzamide;
4-{6-[(6-fluoro-1,2,3,4-tetrahydroquinolin-1-yl)carbonyl]imidazo[1,2-a]pyrazin-3-yl}benzamide;
N-(4-fluorophenyl)-N-methyl-3-[4-(1,3,4-thiadiazol-2-yl)phenyl]imidazo[1,2-a]pyrazine-6-carboxamide;
4-{6-[(6-fluoro-1,2,3,4-tetrahydroquinolin-1-yl)carbonyl]imidazo[1,2-a]pyrazin-3-yl}-N-methylbenzamide;
N-(4-{6-[(7-fluoro-3,3-dimethyl-3,4-dihydro-2H-1,4-benzoxazin-4-yl)carbonyl]imidazo[1,2-a]pyrazin-3-yl}phenyl)acetamide;
N-methyl-4-{6-[N-methyl(4-fluorobenzene)amido]imidazo[1,2-a]pyrazin-3-yl}benzamide;
N-methyl-N-(4-sulfamoylphenyl)-3-[4-(trifluoromethyl)phenyl]imidazo[1,2-a]pyrazine-6-carboxamide;
4-{6-[(7-fluoro-3,4-dihydro-2H-1,4-benzoxazin-4-yl)carbonyl]imidazo[1,2-a]pyrazin-3-yl}benzamide;
4-{6-[N-methyl(4-fluorobenzene)amido]imidazo[1,2-a]pyrazin-3-yl}benzamide;
4-fluoro-N-methyl-N-(3-{1H-pyrrolo[2,3-b]pyridin-5-yl}imidazo[1,2-a]pyrazin-6-yl)benzamide;
1-{[3-(2,3-dihydro-1-benzofuran-5-yl)imidazo[1,2-a]pyrazin-6-yl]carbonyl}-6-fluoro-1,2,3,4-tetrahydroquinoline;
3-(4-carbamoylphenyl)-N-methyl-N-(5-methylpyridin-2-yl)imidazo[1,2-a]pyrazine-6-carboxamide;
4-{6-[(7-fluoro-3,4-dihydro-2H-1,4-benzoxazin-4-yl)carbonyl]imidazo[1,2-a]pyrazin-3-yl}-N-methylbenzamide;
6-fluoro-1-{[3-(3-methanesulfonylphenyl)imidazo[1,2-a]pyrazin-6-yl]carbonyl}-1,2,3,4-tetrahydroquinoline;
3-[6-(4-acetylpiperazin-1-yl)pyridin-3-yl]-N-(4-cyanophenyl)-N-methylimidazo[1,2-a]pyrazine-6-carboxamide;
6-fluoro-1-{[3-(4-methanesulfonylphenyl)imidazo[1,2-a]pyrazin-6-yl]carbonyl}-1,2,3,4-tetrahydroquinoline;
1-{[3-(1-ethyl-1H-pyrazol-4-yl)imidazo[1,2-a]pyrazin-6-yl]carbonyl}-6-fluoro-1,2,3,4-tetrahydroquinoline;
N-(4-cyanophenyl)-N-methyl-3-[6-(piperazin-1-yl)pyridin-3-yl]imidazo[1,2-a]pyrazine-6-carboxamide;
N-(4-cyanophenyl)-3-(furan-2-yl)-N-methylimidazo[1,2-a]pyrazine-6-carboxamide;
N-(4-cyanophenyl)-3-(furan-2-yl)-N-methylimidazo[1,2-a]pyrazine-6-carboxamide;
6-fluoro-1-({3-[2-(piperazin-1-yl)pyridin-4-yl]imidazo[1,2-a]pyrazin-6-yl}carbonyl)-1,2,3,4-tetrahydroquinoline;
N-(4-chlorophenyl)-N-methyl-3-[4-(1,3,4-thiadiazol-2-yl)phenyl]imidazo[1,2-a]pyrazine-6-carboxamide;
N-methyl-3-[4-(methylcarbamoyl)phenyl]-N-(5-methylpyridin-2-yl)imidazo[1,2-a]pyrazine-6-carboxamide;
N-cyclohexyl-4-{6-[(6-fluoro-1,2,3,4-tetrahydroquinolin-1-yl)carbonyl]imidazo[1,2-a]pyrazin-3-yl}benzamide;
4-fluoro-N-methyl-N-[3-(1-methyl-1H-indazol-5-yl)imidazo[1,2-a]pyrazin-6-yl]benzamide;
1-[4-(5-{6-[(6-fluoro-1,2,3,4-tetrahydroquinolin-1-yl)carbonyl]imidazo[1,2-a]pyrazin-3-yl}pyridin-2-yl)piperazin-1-yl]ethan-1-one;
6-fluoro-1-({3-[3-(morpholin-4-ylmethyl)phenyl]imidazo[1,2-a]pyrazin-6-yl}carbonyl)-1,2,3,4-tetrahydroquinoline;
N-(5-methylpyridin-2-yl)-3-[4-(trifluoromethyl)phenyl]imidazo[1,2-a]pyrazine-6-carboxamide;
N-(4-ethylpyridin-2-yl)-N-methyl-3-[4-(trifluoromethyl)phenyl]imidazo[1,2-a]pyrazine-6-carboxamide;
N-(4-cyanophenyl)-N-methyl-3-{4-[5-(methylamino)-1,3,4-thiadiazol-2-yl]phenyl}imidazo[1,2-a]pyrazine-6-carboxamide;
4-(6-{[(4-chlorophenyl)(methyl)amino]methyl}imidazo[1,2-a]pyrazin-3-yl)benzamide;
N-(4-fluorophenyl)-N-methyl-3-{4-[5-(methylamino)-1,3,4-oxadiazol-2-yl]phenyl}imidazo[1,2-a]pyrazine-6-carboxamide;
4-[({3-[4-(difluoromethoxy)phenyl]imidazo[1,2-a]pyrazin-6-yl}methyl)(methyl)amino]benzonitrile;
N-(4-chlorophenyl)-N-methyl-3-(1-methyl-1H-indazol-5-yl)imidazo[1,2-a]pyrazine-6-carboxamide;
N-(4-cyanophenyl)-N-methyl-3-[4-(1H-pyrazol-5-yl)phenyl]imidazo[1,2-a]pyrazine-6-carboxamide;
methyl N-(4-{6-[(4-cyanophenyl)(methyl)carbamoyl]imidazo[1,2-a]pyrazin-3-yl}phenyl)carbamate;
N-(4-chlorophenyl)-N-methyl-3-{4-[5-(methylamino)-1,3,4-oxadiazol-2-yl]phenyl}imidazo[1,2-a]pyrazine-6-carboxamide;
N-(4-cyanophenyl)-N-methyl-3-[4-(1H-pyrazol-3-yl)phenyl]imidazo[1,2-a]pyrazine-6-carboxamide;
N-(4-cyanophenyl)-3-(4-acetamidophenyl)-N-methylimidazo[1,2-a]pyrazine-6-carboxamide;
3-(4-carbamoylphenyl)-N-(4-chlorophenyl)-N-methylimidazo[1,2-a]pyrazine-6-carboxamide;
N-(4-cyanophenyl)-N-methyl-3-(1-methyl-1H-indazol-5-yl)imidazo[1,2-a]pyrazine-6-carboxamide;
N-[3-(6-acetamidopyridin-3-yl)imidazo[1,2-a]pyrazin-6-yl]-4-fluoro-N-methylbenzamide;
N-(6-chloropyridin-3-yl)-N-methyl-3-(1-methyl-1H-indazol-5-yl)imidazo[1,2-a]pyrazine-6-carboxamide;
N-(4-fluoropyridin-2-yl)-N-methyl-3-[4-(trifluoromethyl)phenyl]imidazo[1,2-a]pyrazine-6-carboxamide;
N-methyl-N-(5-methylpyridin-2-yl)-3-[4-(trifluoromethyl)phenyl]imidazo[1,2-a]pyrazine-6-carboxamide;
N-(2-methoxypyridin-3-yl)-N-methyl-3-[4-(trifluoromethyl)phenyl]imidazo[1,2-a]pyrazine-6-carboxamide;
N-(4-cyanophenyl)-N-methyl-3-[4-(1,3,4-thiadiazol-2-yl)phenyl]imidazo[1,2-a]pyrazine-6-carboxamide;
(1,1-dioxido-2H-benzo[b][1,4]thiazin-4(3H)-yl)(3-(4-(trifluoromethyl)phenyl)imidazo[1,2-a]pyrazin-6-yl)methanone;
4-{6-[N-methyl(4-cyanobenzene)amido]imidazo[1,2-a]pyrazin-3-yl}benzamide;
N-(2-chloro-1,3-thiazol-5-yl)-N-methyl-3-[4(trifluoromethyl)phenyl]imidazo[1,2-a]pyrazine-6-carboxamide;
4-({3-[4-(trifluoromethyl)phenyl]imidazo[1,2-a]pyrazin-6-yl}carbonyl)-1,2,3,4-tetrahydroquinoxalin-2-one;
N-methyl-N-{3-[4-(trifluoromethyl)phenyl]imidazo[1,2-a]pyrazin-6-yl}pyrimidine-5-carboxamide;
N-methyl-N-(6-phenylpyridin-3-yl)-3-[4-(trifluoromethyl)phenyl]imidazo[1,2-a]pyrazine-6-carboxamide;
N-methyl-N,3-bis[4-(trifluoromethyl)phenyl]imidazo[1,2-a]pyrazine-6-carboxamide;
N-(4-methanesulfonylphenyl)-N-methyl-3-[4-(trifluoromethyl)phenyl]imidazo[1,2-a]pyrazine-6-carboxamide;
N-(3-chloro-4-cyanophenyl)-N-methyl-3-[4-(trifluoromethyl)phenyl]imidazo[1,2-a]pyrazine-6-carboxamide; and
N-(4-cyano-2-methoxyphenyl)-N-methyl-3-[4-(trifluoromethyl)phenyl]imidazo[1,2-a]pyrazine-6-carboxamide.

9. A pharmaceutical composition comprising at least one compound of claim 1 or a pharmaceutically acceptable salt, or stereoisomer thereof, and a pharmaceutically acceptable carrier, diluent or excipient.

10. The pharmaceutical composition according to claim 9 further comprising a second agent, wherein the second agent is an antimalarial drug selected from artemisinin, artemether, artesunate, arteflene, dihydroartemisinin, chlorproguanil, trimethoprim, chloroquine, quinine, mefloquine, amodiaquine, atovaquone, proguanil, lumefantrine, piperaquine, pyronaridine, halofantrine, pyrimethamine-sulfadoxine, quinacrine, pyrimethamine-dapsone, quinidine, amopyroquine, sulphonamides, primaquine, ferroquine, tafenoquine, arterolane, and pyronaridine.

11. A method for treating, preventing, inhibiting, ameliorating, or eradicating the pathology and/or symptomology of a disease caused by a Plasmodium parasite, comprising administering to a subject a therapeutically effective amount of a compound according to claim 1, wherein the administering may optionally be in combination with a second agent.

12. The method according to claim 11, wherein the disease is malaria.

13. The method according to claim 11, wherein the Plasmodium parasite is at the blood stages or at the hepatic stages.

14. The method according to claim 11, wherein the Plasmodium parasite is selected from group consisting of Plasmodium falciparum, Plasmodium vivax, Plasmodium ovale, and Plasmodium malaria.

15. The method according to claim 11, wherein the second agent is selected from a kinase inhibitor, an anti-malarial drug and an anti-inflammatory agent, and wherein the anti-malarial drug is selected from artemisinin, artemether, artesunate, arteflene, dihydroartemisinin, chlorproguanil, trimethoprim, chloroquine, quinine, mefloquine, amodiaquine, atovaquone, proguanil, lumefantrine, piperaquine, pyronaridine, halofantrine, pyrimethamine-sulfadoxine, quinacrine, pyrimethamine-dapsone, quinidine, amopyroquine, sulphonamides, primaquine, ferroquine, tafenoquine, arterolane, and pyronaridine.

16. A method for treating, preventing, inhibiting, ameliorating, or eradicating the pathology and/or symptomology of a disease caused by a Plasmodium parasite, comprising administering to a subject a therapeutically effective amount of a pharmaceutical composition according to claim 9, wherein the administering may optionally be in combination with a second agent.

17. The method according to claim 16, wherein the disease is malaria.

18. The method according to claim 17, wherein the Plasmodium parasite is at the blood stages or at the hepatic stages.

19. The method according to claim 18, wherein the Plasmodium parasite is selected from group consisting of Plasmodium falciparum, Plasmodium vivax, Plasmodium ovale, and Plasmodium malaria.

20. The method according to claim 16, wherein the second agent is selected from a kinase inhibitor, an anti-malarial drug and an anti-inflammatory agent, and wherein the anti-malarial drug is selected from artemisinin, artemether, artesunate, arteflene, dihydroartemisinin, chlorproguanil, trimethoprim, chloroquine, quinine, mefloquine, amodiaquine, atovaquone, proguanil, lumefantrine, piperaquine, pyronaridine, halofantrine, pyrimethamine-sulfadoxine, quinacrine, pyrimethamine-dapsone, quinidine, amopyroquine, sulphonamides, primaquine, ferroquine, tafenoquine, arterolane, and pyronaridine.
